(19) United States
(12) Patent Application Publication
(10) Pub. No.: US 2024/0423973 A1
(43) Pub. Date: Dec. 26, 2024

(54) COMPOUNDS, PHARMACEUTICAL COMPOSITIONS, AND METHODS FOR THE TREATMENT, PREVENTION, OR MANAGEMENT OF HYPERPROLIFERATIVE DISORDERS

(71) Applicant: VINCERX PHARMA GMBH, Moheim am Rhein (DE)

(72) Inventors: Hans-Georg LERCHEN, Leverkusen (DE); Beatrix STELTE-LUDWIG, Wülfrath (DE); Mareike WIEDMANN, Monheim am Rhein (DE); Anne-Sophie REBSTOCK, Champagne au Mont d'Or (FR); Johannes KOEBBERLING, Neuss (DE); Harvey WONG, Vancouver, BC (CA)

(21) Appl. No.: 18/698,339

(22) PCT Filed: Oct. 3, 2022
(86) PCT No.: PCT/IB2022/000558

§ 371 Date: Apr. 3, 2024

Related U.S. Application Data
(60) Provisional application No. 63/252,119, filed on Oct. 4, 2021.

(51) Int. Cl. A61K 31/4745; A61K 47/64; A61P 35/00; C07D 491/22

(52) U.S. Cl. CPC A61K 31/4745; A61K 47/64; A61P 35/00; C07D 491/22

(57) ABSTRACT

Disclosed herein are compounds, pharmaceutical compositions, and methods for treating, preventing or managing diseases and conditions including hyperproliferative disorders such as cancer in humans and other mammals. Compounds disclosed herein are cytotoxic or cytostatic (e.g., 7-ethyl camptothecin) prodrugs, conjugated to an integrin binding moiety via cleavable linkers and/or functional spacers.

### CROSS-REFERENCE

This International Patent Application claims the benefit of U.S. Provisional Patent Application No. 63/252,119, filed Oct. 4, 2021, which is incorporated herein by reference in its entirety.

### BACKGROUND OF THE INVENTION

This application relates to compounds, pharmaceutical compositions, processes for preparation thereof, and the use thereof for treating, preventing, or managing diseases and conditions including hyperproliferative disorders, such as, cancer in humans and other mammals.

### SUMMARY OF THE INVENTION

The present invention relates to novel pharmaceutical compounds (or “conjugates”) comprising of one or more αᵥβ3 integrin binders, novel linking units containing one or more alkyl amino groups at different positions in the linker as well as peptide sequences cleavable by elastase, and a cytotoxic element (e.g., 7-ethyl camptothecin), the processes for preparation thereof, to the use thereof for treating, preventing or managing diseases and conditions including hyperproliferative disorders such as cancer in humans and other mammals.

The current invention describes conjugates with small molecule binders, which release the drug extracellularly in the tumor microenvironment (TME) upon cleavage by enzymes present in TME. Compounds described herein may comprise small molecule binders (e.g., integrin binder), conjugated via an enzymatically cleavable linker and/or a polymeric linker to a cytotoxic element (e. g. 7-ethyl camptothecin) which, in some embodiments, is retained in an acidic TME.

In one aspect, provided herein is a compound, or a pharmaceutically acceptable salt thereof, having a structure represented by Formula (I):

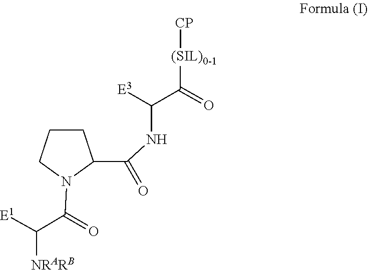

wherein:
CP is a cytotoxic or cytostatic group;
SIL is a self-immolative linker;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
R^{A} is hydrogen or C₁₋₆ alkyl;
R^{B} is -L¹-A¹(L²-(IN))(L³-(IN)), -L⁴-IN, L⁵-IN, L⁷-IN, or -L¹-A¹(L²-(IN))(L³-(MOD));
   wherein:
   each of L¹, L², L³, and L⁵ is, independently, a bivalent linker;
   L⁴ is a bivalent polyamine or polyamide linker;
L⁷ is a substituted or unsubstituted C₁₋₆₀ alkyl, or substituted or unsubstituted heteroalkyl comprising 4-12 heteroalkyl units, wherein each Z group is independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, and —NHS(O)₂NHC(O)—;
   A¹ is a trivalent linker;
   IN is in each instance, independently, an integrin binder;
   MOD is a physicochemical or pharmacokinetic modulator;
      with the proviso that when R^{B} is L⁵, E^{t} is E²; or E³ is —CH₃;
   wherein E² is —CH₂C(O)OR¹ or —CH₂CH₂C(O)OR¹;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆ alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl;
or a pharmaceutically acceptable salt thereof, or a stereoisomer or mixture of stereoisomers thereof.

In some embodiments, provided herein is a compound, or a pharmaceutically acceptable salt thereof, having a structure represented by Formula (I-A), Formula (I-B), Formula (I-C), Formula (I-D), or Formula (I-E):

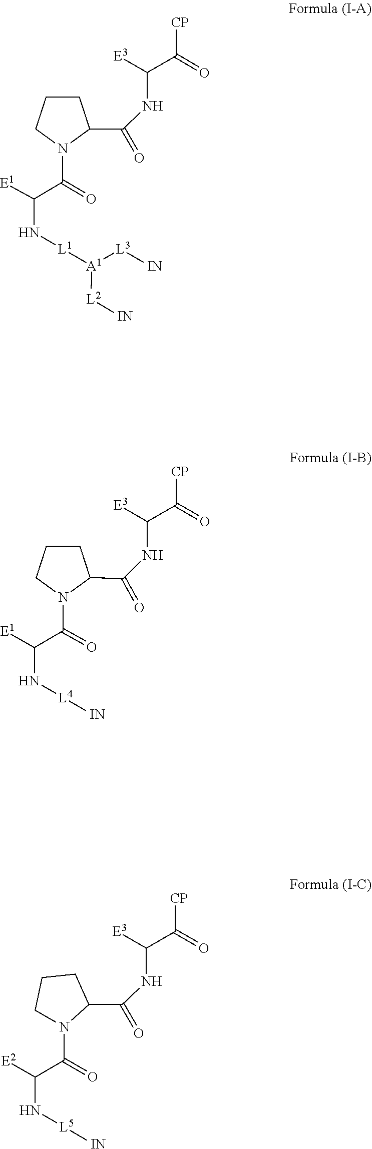

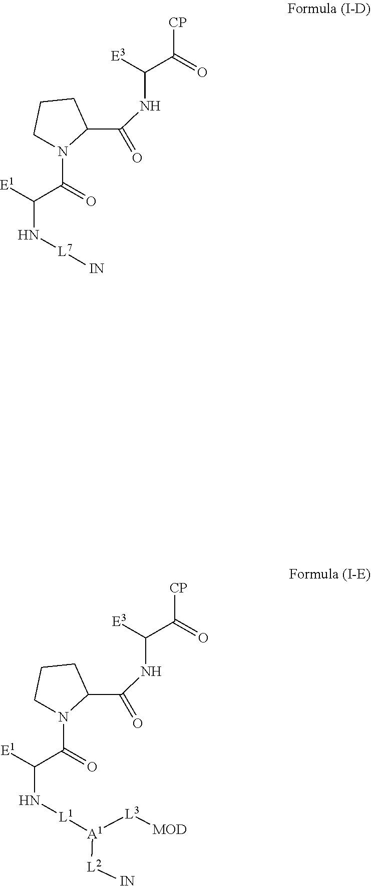

or a pharmaceutically acceptable salt thereof, or a stereoisomer or mixture of stereoisomers thereof, wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH, or —CH₂CH₂C(O)OR¹;
E² is —CH₂C(O)OR¹ or —CH₂CH₂C(O)OR¹;
E³ is —CH₃, —CH(CH₃)₂, —CH₂CH(CH₃)₂, or —CH(CH₃)CH₂CH₃;
IN is, in each instance, independently, an integrin binder;
A¹ is a trivalent linker;
each of L¹, L², L³, and L⁵ is independently a bivalent linker;
L⁴ is a bivalent polyamine or polyamide linker;
L⁷ is a substituted or unsubstituted C₁₋₃₀ alkyl, or substituted or unsubstituted heteroalkyl containing 4-12 Z-groups; wherein each Z group is independently —(O—C₂₋₆ alkyl)-, —(NH—C₂₋₆alkyl)-, or —(N(C₁₋₃ alkyl)-C₂₋₆alkyl)-;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and IN is an integrin binder; and
MOD is a physicochemical or pharmacokinetic modulating group (e.g., —COOH).

In some embodiments, the compound is cleaved by neutrophil elastase. In some embodiments, CP is a pentacyclic compound. In some embodiments, CP is a topoisomerase inhibitor. In some embodiments, CP is bonded via an ester linkage. In some embodiments, CP is camptothecin or a radical thereof.

In some embodiments, provided herein is a compound, or a pharmaceutically acceptable salt thereof, having a structure represented by Formula (II):

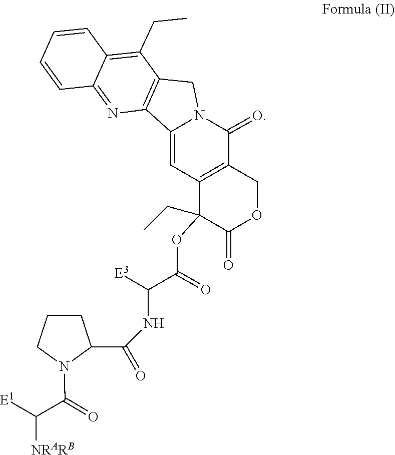

In some embodiments, provided herein is a compound, or a pharmaceutically acceptable salt thereof, having a structure represented by Formula (II-A), Formula (II-B), Formula (II-C), Formula (II-D), or Formula (II-E):

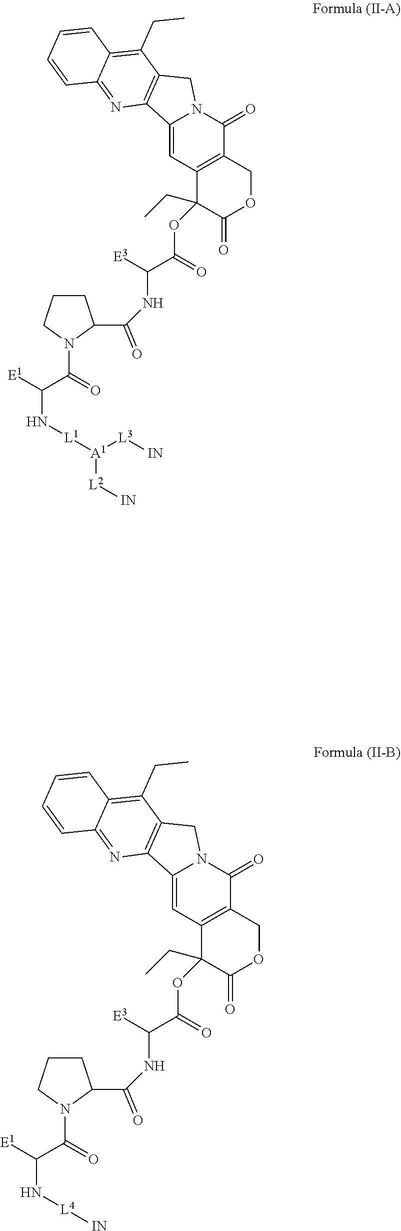

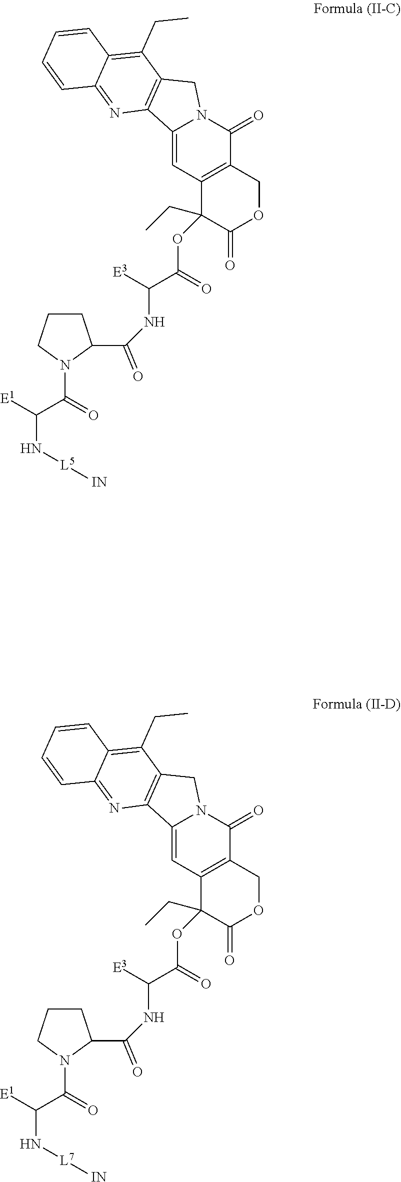

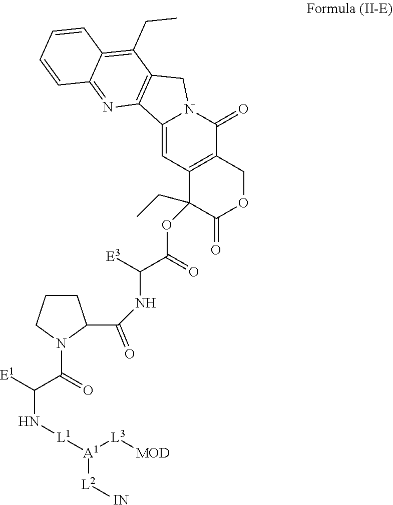

wherein:
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E² is —CH₂C(O)OR¹ or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each of L¹, L², L³, and L⁵ is independently a bivalent linker;
L⁴ is a bivalent polyamine or polyamide linker;
L⁷ is a substituted or unsubstituted C₁₋₃₀ alkyl, or substituted or unsubstituted heteroalkyl containing 4-12 Z-groups; wherein each Z group is independently —(O—C₂₋₆ alkyl)-, —(NH—C₂₋₆alkyl)-, or —(N(C₁₋₃ alkyl)-C₂₋₆alkyl)-;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein; L⁶ is a bivalent linker;
MOD is a physicochemical or pharmacokinetic modulating group; and
IN is, in each instance, independently, an integrin binder.

In some embodiments, provided herein is a compound, or a pharmaceutically acceptable salt thereof, having a structure represented by Formula (III-A), Formula (III-B), Formula (III-C), Formula (III-D), or Formula (III-E):

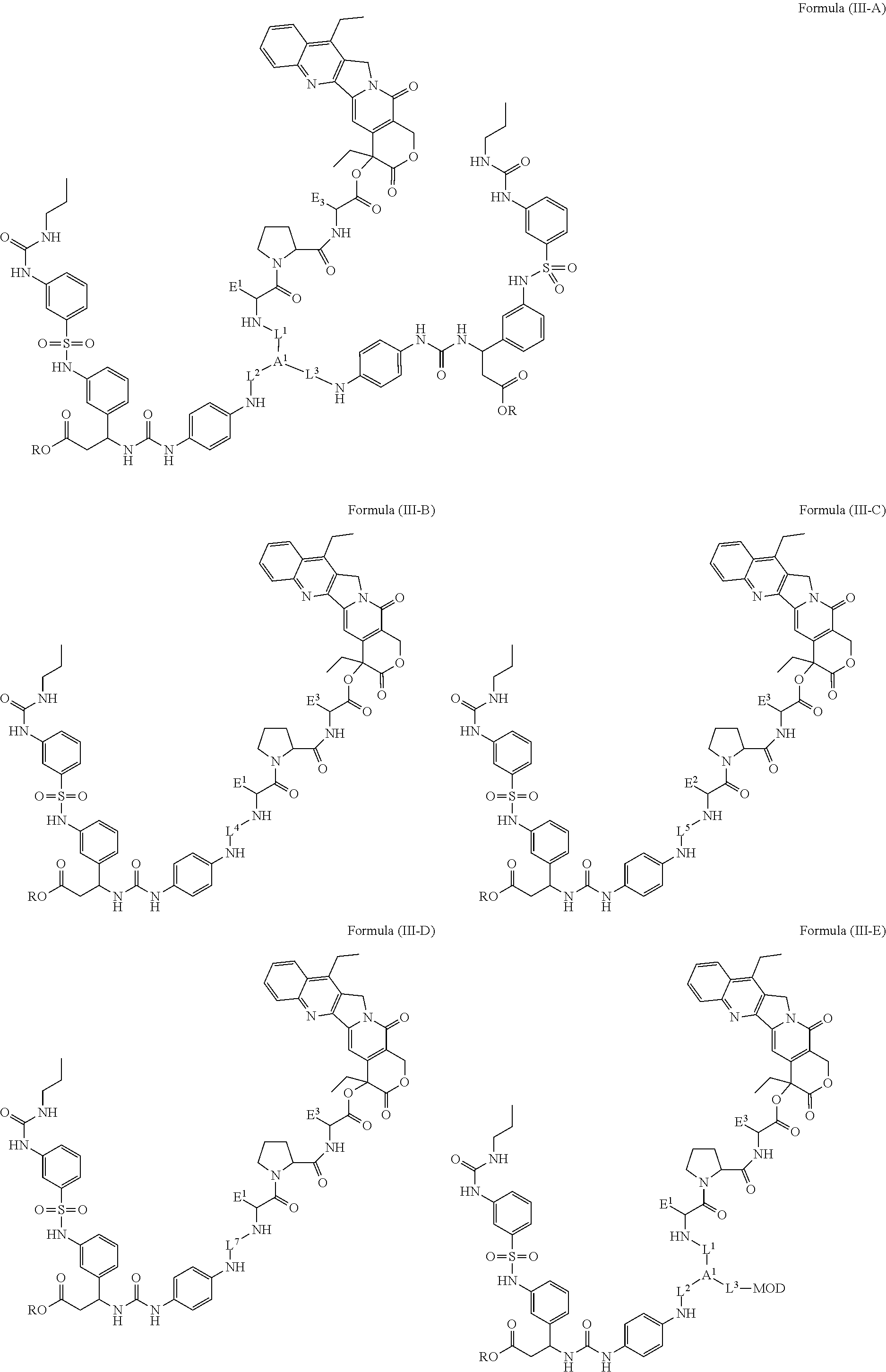

or a pharmaceutically acceptable salt thereof, or a stereoisomer or mixture of stereoisomers thereof, wherein:
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E² is —CH₂C(O)OR¹ or —CH₂CH₂C(O)OR¹;
E³ is —CH₃, —CH(CH₃)₂, —CH₂CH(CH₃)₂, or —CH(CH₃)CH₂CH₃;
IN is, in each instance, independently, an integrin binder;
A¹ is a trivalent linker;
each of L¹, L², L³, and L⁵ is independently a bivalent linker;
L⁴ is a bivalent polyamine or polyamide linker;
L⁷ is a substituted or unsubstituted C₁₋₃₀ alkyl, or substituted or unsubstituted heteroalkyl containing 4-12 Z-groups; wherein each Z group is independently —(O—C₂₋₆ alkyl)-, —(NH—C₂₋₆alkyl)-, or —(N(C₁₋₃ alkyl)-C₂₋₆alkyl)-;
R is hydrogen or a substituted or unsubstituted C₁₋₁₂ alkyl; and
R¹ is a substituted or unsubstituted C₁₋₁₂ alkyl;
wherein if R or R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and
MOD is a physicochemical or pharmacokinetic modulating group.

In another aspect, provided herein is a pharmaceutical composition comprising a compound disclosed herein, and at least one pharmaceutically acceptable excipient.

In still another aspect, described herein is a compound, or a pharmaceutically acceptable salt thereof, or a stereoisomer or mixture of stereoisomers thereof, or a pharmaceutical composition thereof, for use as a medicament. In still another aspect, described herein is a compound, or a pharmaceutically acceptable salt thereof, or a stereoisomer or mixture of stereoisomers thereof, or a pharmaceutical composition thereof, for use in a method of treating a disease or disorder disclosed herein. In some embodiments, the disease or disorder is a hyperproliferative disorder. In some embodiments, the disease or disorder is a cancer.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 panel (a) shows the stability of Compound B1 in rat plasma; and panel (b) shows the stability of Compound B1 in buffer at pH 7.4.

FIG. 2 shows the stability of Prodrug Compound C2 in rat plasma at 37° C.

FIG. 3 shows the stability of Prodrug Compound C3 in rat plasma at 37° C.

FIG. 4: avb3 binding of selected examples and EC50 values

FIG. 5: Tumor growth inhibition after treatment with indicated compounds in the MX1 TNBC mouse model (tumor area and T/C ratio)

### DETAILED DESCRIPTION OF THE INVENTION

The present invention relates to novel pharmaceutical compounds comprising one or more αᵥβ3 integrin antagonists, novel linker units containing one or more N-alkyl amino groups or one or more oxygens at different positions in the linker as well as peptide sequences cleavable by tumor-associated enzymes such as neutrophil elastase, and a cytotoxic component (e.g., a hexacyclic topoisomerase inhibitor), the processes for preparation thereof, to the use thereof for treating, preventing or managing diseases and conditions including hyperproliferative disorders such as cancer in humans and other mammals.

Chemotherapy in cancer is accompanied by usually serious side effects which are to be attributed to the toxic action of chemotherapeutics on proliferating cells of other tissue types rather than tumor tissue. For many years, scientists have occupied themselves with the problem of improving the selectivity of active compounds employed. A frequently followed approach is the synthesis of prodrugs which are released more or less selectively in the target tissue, for example, by change of the pH (DE4229903), by enzymes (e.g. glucuronidases; EP0511917 and EP0595133) or by antibody-enzyme conjugates (WO198807378; U.S. Pat. No. 4,975,278; EP0595133). A problem with these approaches is, inter alia, the lack of stability of the conjugates in other tissues and organs, and, in particular, the ubiquitous active compound distribution which follows the extracellular release of active compound in the tumor tissue.

20(S)-Camptothecin is a pentacyclic alkaloid which was isolated in 1966 by Wall et al. (J. Am. Chem. Soc. 88, 3888 (1966)). It has a high active antitumor potential in numerous in-vitro and in-vivo tests. Unfortunately, however, the realization of the promising potential in the clinical investigation phase failed because of toxicity and solubility problems.

By opening of the E ring lactone and formation of the sodium salt, a water-soluble compound was obtained which is in a pH-dependent equilibrium with the ring-closed form. Here too, clinical studies have not led to success.

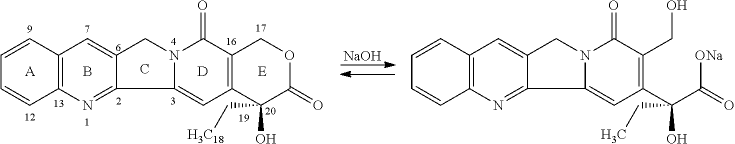

About 20 years later, it was found that the biological activity is to be attributed to enzyme inhibition of topoisomerase I. Since then, the research activities have again been increased in order to find a camptothecin derivative which is more soluble, better tolerated and which is active in vivo.

For improvement of the water solubility, salts of A-ring- and B-ring-modified camptothecin derivatives and of 20-O-acyl derivatives with ionizable groups have been described (U.S. Pat. No. 4,943,579). The latter prodrug concept was later also transferred to modified camptothecin derivatives (WO 1996/002546). The described 20-O-acyl prodrugs, however, have a very short half-life in vivo and are very rapidly cleaved to give the parent structure.

A large number of camptothecin derivatives have been investigated in preclinical and clinical studies; from those, irinotecan, topotecan and belotecan have successfully been approved (Li et al, Am J Cancer Res 2017; 7(12):2350-2394). Some such derivatives (A-W) are listed below.

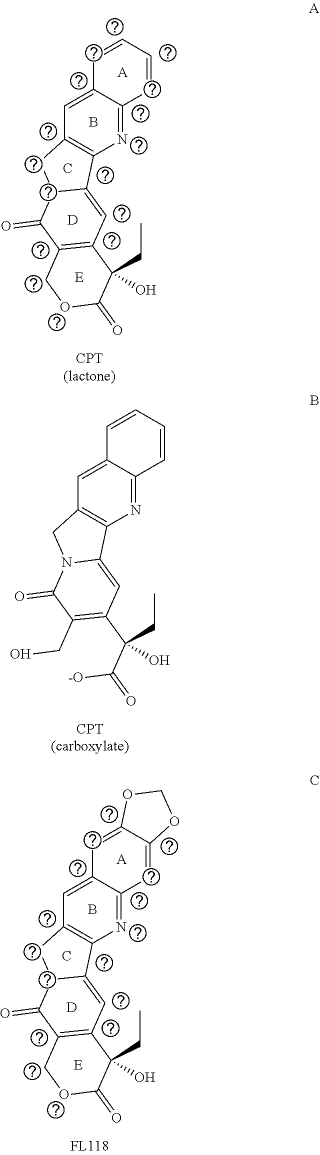

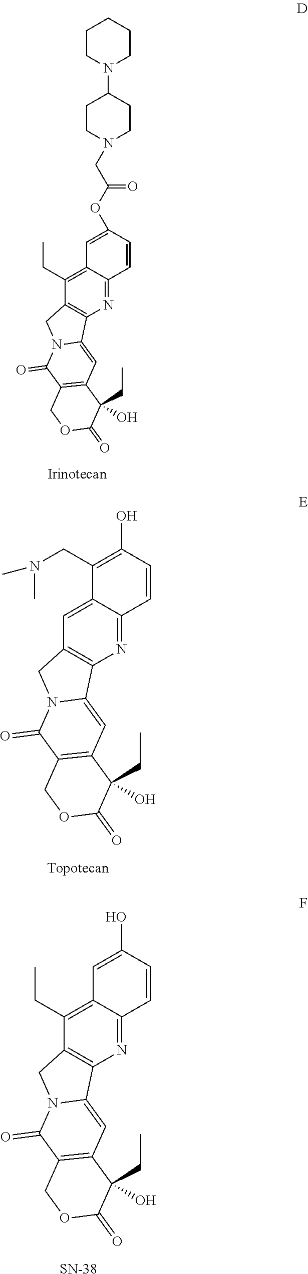 [SMILES: CCc1c2c(nc3ccc(OC(=O)CN4CCC(N5CCCCC5)CC4)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1 ; CCc1c2c(nc3ccc(O)cc13)-c1cc3c(c(=O)n1C2)COC(=O)[C@]3(O)CC ; "SN-38"]

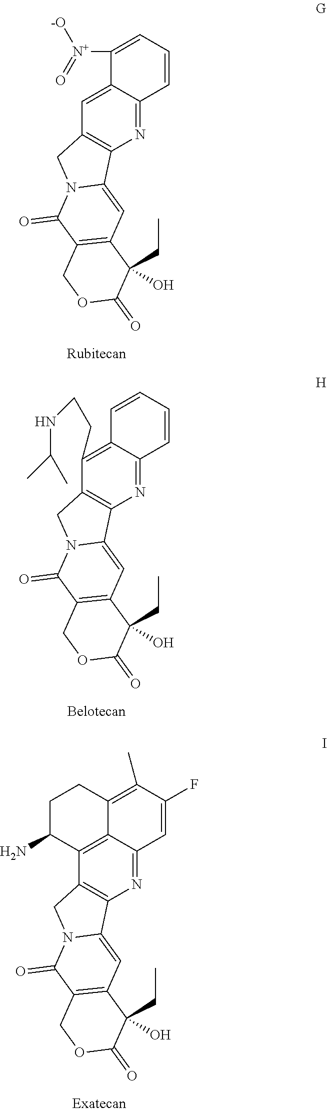

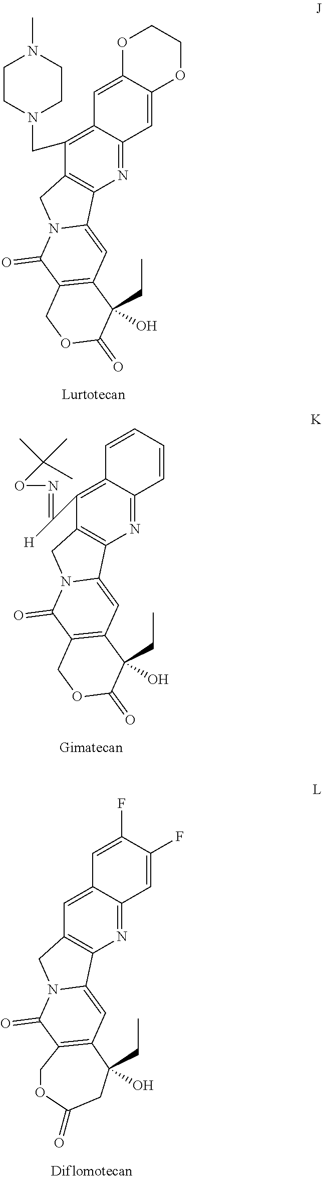 [SMILES: CC[C@@]1(O)C(=O)OCc2c1cc1n(c2=O)Cc2c-1nc1cc3c(cc1c2CN1CCN(C)CC1)OCCO3 ; CC[C@@]1(O)C(=O)OCc2c1cc1n(c2=O)Cc2c-1nc1ccccc1c2/C=N/OC(C)(C)C ; CC[C@@]1(O)CC(=O)OCc2c1cc1n(c2=O)Cc2cc3cc(F)c(F)cc3nc2-1]

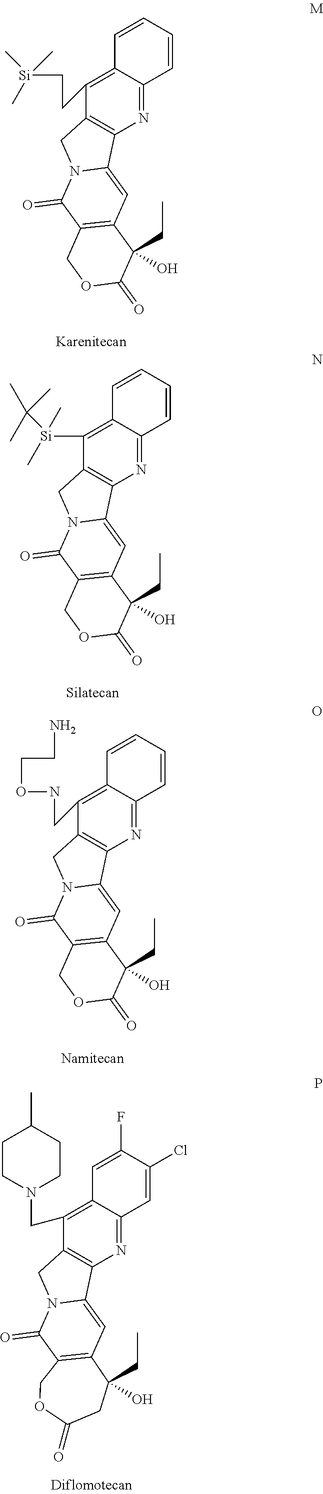 [SMILES: CC[C@@]1(O)C(=O)OCc2c1cc1n(c2=O)Cc2c-1nc1ccccc1c2CC[Si](C)(C)C ; CC[C@@]1(O)C(=O)OCc2c1cc1n(c2=O)Cc2c-1nc1ccccc1c2[Si](C)(C)C(C)(C)C ; CC[C@@]1(O)C(=O)OCc2c1cc1n(c2=O)Cc2c-1nc1ccccc1c2CNOCCN ; CC[C@@]1(O)CC(=O)OCc2c1cc1n(c2=O)Cc2c-1nc1cc(Cl)c(F)cc1c2CN1CCC(C)CC1]

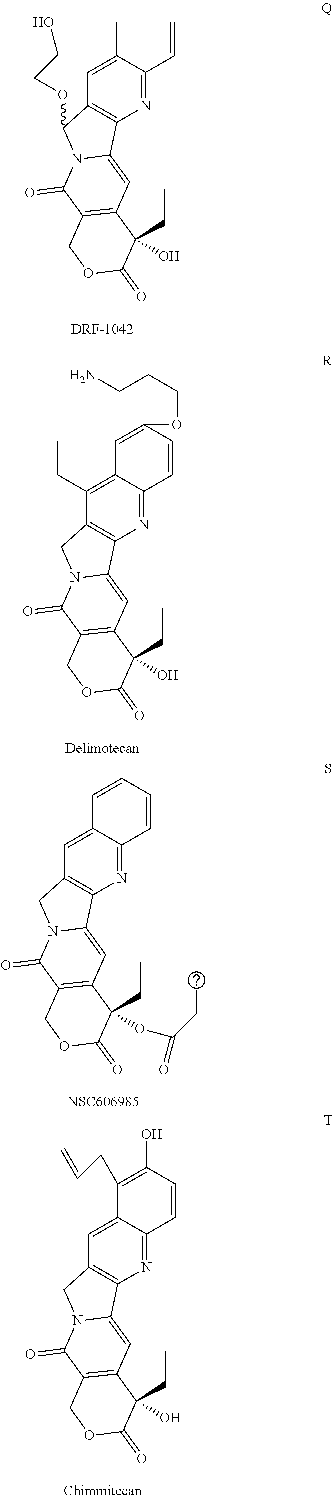 [SMILES: C=Cc1nc2c(cc1C)C(OCCO)n1c-2cc2c(c1=O)COC(=O)[C@]2(O)CC ; CCc1c2c(nc3ccc(OCCCN)cc13)-c1cc3c(c(=O)n1C2)COC(=O)[C@]3(O)CC ; CCC(=O)O[C@]1(CC)C(=O)OCc2c1cc1n(c2=O)Cc2cc3ccccc3nc2-1 ; C=CCc1c(O)ccc2nc3c(cc12)Cn1c-3cc2c(c1=O)COC(=O)[C@]2(O)CC]

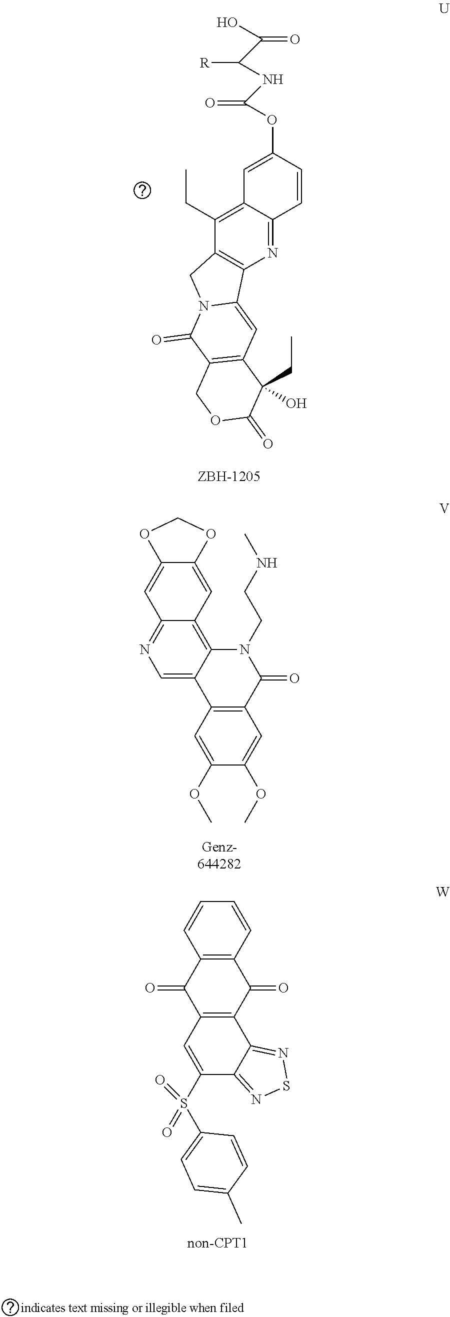

Improving the therapeutic window of cytotoxic agents such as camptothecin and its derivatives remains a challenge.

Integrins are heterodimeric transmembrane proteins found on the surface of cells, which play an important part in the adhesion of the cells to an extracellular matrix. They recognize extracellular glycoproteins such as fibronectin or vitronectin on the extracellular matrix via the RGD sequence occurring in these proteins (RGD is the single-letter code for the amino acid sequence arginine-glycine-aspartate).

In general, integrins such as, the vitronectin receptor, which is also called the αᵥβ3 receptor, or alternatively the αᵥβ5 receptor or the GpIIb/IIIa receptor play an important part in biological processes such as cell migration, angiogenesis and cell-matrix adhesion and thus for diseases in which these processes are crucial steps. Cancer, osteoporosis, arteriosclerosis, restenosis and ophthalmia may be mentioned by way of example.

The αᵥβ3 receptor occurs, for example, in large amounts on growing endothelial cells and makes possible their adhesion to an extracellular matrix. The αᵥβ3 receptor thus plays an important part in angiogenesis, i. e. the formation of new blood vessels, which is a crucial prerequisite for tumor growth and metastasis formation in carcinomatous disorders.

It was possible to show that the blockade of the above-mentioned receptors is an important starting point for the treatment of disorders of this type. If the adhesion of growing endothelial cells to an extracellular matrix is suppressed by blocking their corresponding integrin receptors, for example, by a cyclic peptide or a monoclonal antibody, angiogenesis does not occur, which leads to a stoppage or regression of tumor growth (cf., for example, Brooks et al. in Cell 79, 1157-1164 (1994)).

Despite compelling preclinical results demonstrating that the inhibition of integrin has therapeutic potential, clinical trials with integrin inhibitors targeting those integrins have repeatedly failed to demonstrate therapeutic benefits in cancer patients (C. Ruegg et al, Cancers 2019, 11, 978).

WO 1998/010795 describes conjugates in which a molecule targeting tumors is linked to a functional unit such as, for example, a cytostatic or a detectable label such as, for example, a radioactive nuclide. Inter alia, integrin antagonists such as, for example, peptides having the RGD sequence described above are described as molecules targeting tumor or tumor stroma. Doxorubicin is described as an example of a cytostatic which is linked to a molecule of this type addressing tumors.

In the case of the compounds of WO 1998/010795, the linkage is carried out such that the molecule addressing a tumor and the functional unit are directly bonded to one another with retention of their respective properties (cf., for example, p. 56, 1. 17, to p. 58, 1. 10, and Ex. 6). This has the result that these compounds are indeed selectively concentrated in the immediate vicinity of tumor cells by binding of the entity addressing a tumor (in the case of a radical having αᵥβ3 integrin-antagonistic action by binding to the αᵥβ3 integrin receptor which, in particular, is expressed on endothelial cells newly formed by angiogenesis), but on account of the direct combination the functional unit such as, for example, a cytostatic cannot be released into the intracellular space of the tumor tissue.

Fundamentally, the conjugate which on the one hand is selectively concentrated in tumor tissue by the effect of a part addressing αᵥβ3 or αᵥβ5 integrin receptors found in the conjugate, but on the other hand comprises a cytostatic which can be released from the conjugate, should have an increased toxophoric effect on tumor tissue due to the possibility of the more direct action of the cytostatic on the tumor cells compared with the conjugates described in WO 1998/010795. Such a toxophoric effect and tumor selectivity should be even higher, if the release of the cytostatic takes place in the immediate vicinity of the tumor tissue or even in the tumor cells.

In WO 2000/069472 enzyme-activated anti-tumor prodrug compounds are disclosed which can be specifically cleaved by collagenase (IV) and elastase. With respect to linking units cleavable by elastase this application describes that the specific tetrapeptide sequences Ala-Ala-Pro-Val and Ala-Ala-Pro-Nva are suitable. Furthermore, in this reference, no conjugates which comprise a moiety addressing αᵥβ3 integrin receptors and a cytostatic are mentioned. Y. Liu et al. (Mol. Pharmaceutics 2012, 9, 168) describe conjugates of Auristatins linked to an αᵥβ3 integrin targeting moiety via an legumain-cleavable linker.

In EP1238678 conjugates with cytotoxic agents are disclosed which target αᵥβ3 integrins and have peptide linkers which can be specifically cleaved by elastase. With respect to linking units cleavable by elastase this application describes peptide sequences comprising Pro-Val and Pro-Leu. As toxophore moieties camptothecin and a quinolone carboxylic acid are exemplified.

Particular challenges of such conjugates include
sufficient solubility enabling intravenous administration in appropriate vehicles,
high tumor penetration of intact conjugates,
high stability in plasma to avoid systemic de-conjugation,
efficient binding to the targeted receptors in the tumor microenvironment,
efficient cleavage by enzymes present in the tumor microenvironment,
high cellular permeability and low efflux ratio of cleaved toxophore moieties to enhance tumor cell uptake versus re-distribution.

It is therefore an objective to develop conjugates which comprise a moiety addressing tumor targets such as αᵥβ3 integrin receptors and a cytotoxic agent which can be released from the conjugate preferably in tumor microenvironment, where the moiety in the conjugate addressing αᵥβ3 integrin receptors retains its ability to bind to the αᵥβ3 integrin receptor and therefore provides tissue selectivity to such compounds. In addition, cleavability of the conjugates and drug release should be mediated by enzymes present and active in the tumor environment such as neutrophil elastase. Finally, the profile of the toxophore should be suitable for extracellular cleavage and release mechanism. The toxophore should be highly permeable into tumor cells and tissues and not be a substrate of drug transporters.

In WO2020094471 αᵥβ3 integrin conjugates with 7-ethyl camptothecin have been described, which addressed these aspects. Solubility of the conjugates disclosed in WO2020094471 has been achieved with negatively charged carboxy groups and a PEG unit in the linker.

To improve tumor targeting and accumulation in the acidic tumor microenvironment, the present invention relates to conjugates comprising one or more tumor binding molecules such as αᵥβ₃ integrin binding moieties, linker units which can be selectively cleaved by tumor associated enzymes such as neutrophil elastase, spacer units that may beneficially impact the PK profile, and 7-ethyl camptothecin as a cytotoxic payload. Special structural features of such conjugates can be, but are not limited to a polyalkylamine spacer, spacers with carboxylate side chains, multimeric binding achieved with a dendrimer approach with two or more binding molecules in the conjugate, and/or slow release prodrug residues to modulate (i) the PK of the conjugate, (ii) drug release from the conjugate, and/or (iii) the PK of the released free drug. Such conjugates have a tumor-specific action because of linkage to tumor homing molecules such as αᵥβ₃ integrin binders via preferred linking units which can selectively be cleaved by tumor associated enzymes such as neutrophil elastase. The preferred linking units provide sufficient stability of the compound/conjugate in biological media, e.g. culture medium or serum and, at the same time, the desired intracellular action within tumor tissue as a result of its specific enzymatic or hydrolytic cleavability with release of the drug moiety.

In particular, the compounds of the present invention show one or more of the following features
Modified alkyl spacers interrupted by one or more N-alkyl amino groups with a beneficial impact on retention in tumor microenvironment having an acidic pH and furthermore allowing for high solubility
One or more αᵥβ₃ integrin-binding moieties enabling high or moderate affinity
Modified linkers interrupted by amide bonds and substituted by a physicochemical or pharmacokinetic modifier such as carboxy-substituted side chains with beneficial impact on solubility and DMPK properties
Despite significant steric challenges, these novel linkers appear highly susceptible for cleavage by enzymes present in tumor stroma such as neutrophil elastase
High stability of the conjugates in plasma and cytotoxic activity which is increased in the presence of elastase
Employment of 7-ethyl camptothecin as a particularly suitable toxophore (CT) moiety
Conjugate design by hydroxy lactone acylation with a beneficial impact on lactone ring stability (Drugs Fut 2002, 27(9), 869)
High cellular permeability and low efflux of the payload 7-ethyl camptothecin (as compared e.g. to SN38).

Towards this goal, 7-ethyl camptothecin is particularly preferred as the toxophore moiety in above mentioned conjugates. Provided herein are conjugate of cytotoxic or cytostatic ligands, e.g., 7-ethyl camptothecin, that are designed to address the various challenges that have limited clinical viability of compounds and conjugates previously described in the art. Particularly, the present compounds and conjugates provide linking moieties with functional and beneficial effects, including tumor retention, solubility and PK properties, incorporation of multiple integrin binding units, elongated distance between the toxophore and targeting moiety, and prodrug designs which enhance on-target toxicity while limiting such toxicity issues at off-target sites (e.g., where neutrophil elastase is less prevalent). The compounds and conjugates disclosed herein are clinically advantageous and provide additional elements of utility for at least those reasons described supra, as well as others that will be apparent to those skilled in the art.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Compounds

Cancer cells or tumor microenvironment overexpress certain enzymes, such as, serine proteases, including neutrophil elastase. Conjugates described herein are enzymatically cleavable by serine proteases, including neutrophil elastase. In some embodiments, conjugates described herein are selectively cleavable in the microenvironment of cancer cells, with less or no cleavage taking place e. g. in blood and in healthy cells. In some embodiments, a conjugate described herein is cytotoxic after activation by a tumor-associated enzyme, such as, neutrophil elastase. In some embodiments, a conjugate described herein is non-toxic, in therapeutic concentrations, in the absence of said activation. The cytotoxic agents described herein are inhibitors of topoisomerase. The cytotoxic agent may also be conjugated, e.g., via an enzymatically cleavable linker and a spacer, to an integrin binding moiety. Integrin binding moieties for use in the present disclosure include any agent that can bind to a tumor cell or to extracellular matrix. Examples include, but are not limited to, small molecules, peptides, proteins, and the like. In some embodiments, the integrin binding moiety binds to an integrin that is alpha-v beta-3 (or alternatively: αᵥβ3, aᵥb₃, or even avb3). In some embodiments, a conjugate described herein acts as a prodrug, e.g., an enzymatically or hydrolytically cleavable prodrug, and one of the cleaved components is a cytotoxic or cytostatic agent (e.g., 7-ethyl camptothecin) described herein. In some embodiments, a conjugate described herein is a compound having the formula (I), as defined herein.

In an aspect, provided herein is a compound, or a pharmaceutically acceptable salt thereof; or a stereoisomer or mixture of stereoisomers thereof, having a structure represented by any one of Formulae (01)-(05):

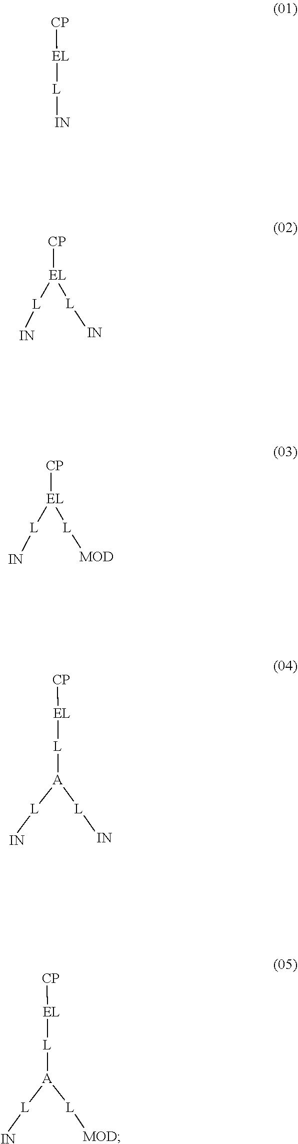

wherein:
CP is a cytotoxic or cytostatic group;
EL is an enzymatically-cleavable linker;
L is a stable linker;
A is a branching group;
TN is an integrin binding group; and
MOD is a physicochemical or pharmacokinetic modulator group.

In another aspect, provided herein are compounds having a structure represented by Formula (A), Formula (B), Formula (C), Formula (D), or Formula (E):

CP-EL-L¹-A¹(L²-IN)(L³-IN) Formula (A)

CP-EL-L⁴-IN Formula (B)

CP-EL-L⁵-IN Formula (C)

CP-EL-L⁷-IN Formula (D)

CP-EL-L1-A1(L2-IN)(L3-MOD) Formula (E)
wherein, in each instance:
CP is a cytotoxic or cytostatic moiety (e.g., a topoisomerase inhibitor (e.g., camptothecin or 7-ethyl camptothecin));
EL is an enzymatically-cleavable linker (e.g., an elastase-cleavable linker);
each L¹, L², L³, and L⁵ is independently a bivalent linker (e.g., a substituted or unsubstituted C₁₋₃₀ alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
L⁴ is a polyamine linker (e.g., polyethyleneimine (“PEI”), (e.g., [—CH₂CH₂NH—]₁₋₂₀ or [—CH₂CH₂NCH₃—]¹⁻²⁰)), a polyamide linker (e.g., polyglycine or polysarcosine (e.g., [—CH₂C(O)NH—]₁₋₂₀ or [—CH₂C(O)NCH₃—]₁₋₂₀)), or a sulfamide linker (e.g., a C₁₋₆₀ alkyl or 1-60 membered heteroalkyl, optionally intersected by one or more sulfamide groups (e.g., —NHS(O)₂NH—, —NHS(O)₂NHC(O)—, or —NHS(O)₂NHC(O)O—) and optionally further interrupted by heteroaryl);
L⁷ is an elongated linker (e.g., a substituted C₁₂₋₃₀ alkyl, or a substituted or unsubstituted heteroalkyl having 15 to 60 (preferably 18 to 36) non-hydrogen atoms (preferably selected from C, N, O, and S));
IN is in each instance, independently, an integrin binder;
MOD is a physicochemical or pharmacokinetic modulating group; and
A¹ is a trivalent linker (e.g., a trivalent radical containing 1 to 100 non-hydrogen atoms, optionally containing alkyl, heteroalkyl, carbocyclic, and/or heterocyclic groups, or any combination thereof).

In another aspect, provided herein are compounds having a structure represented by Formula (I) or Formula (I′):

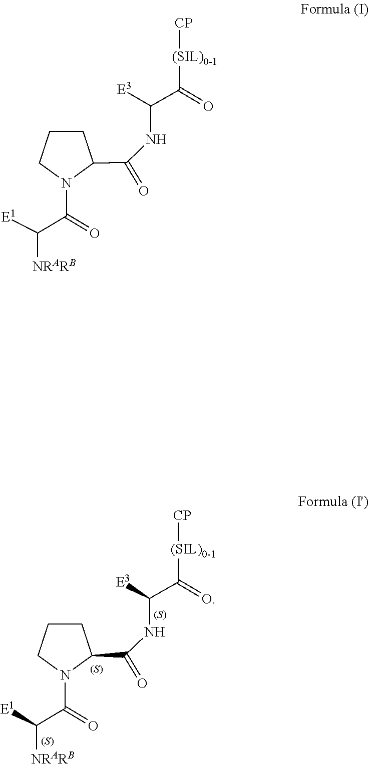

In some embodiments, provided herein are compounds having a structure represented by Formula (I) or Formula (I′):

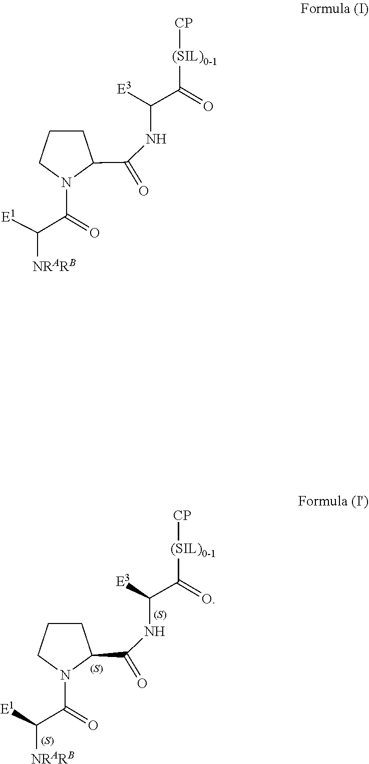

wherein:
CP is a cytotoxic or cytostatic group (e.g., payload);
SIL is a self-immolative linker;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, CH(CH₃)₂, CH₂CH(CH₃)₂, or CH(CH₃)CH₂CH₃;
R^{A} is hydrogen or C₁₋₆ alkyl;
R^{B} is -L¹-A¹(L²-(IN))(L³-(IN)), -L⁴-IN, L⁷-IN, or -L¹-A¹(L²-(IN))(L³-(MOD)); wherein:
   IN is an integrin binder;
   MOD is a physicochemical or pharmacokinetic modulating group;
   A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
   each of L¹, L², L³ and L⁷ is independently a bivalent linker (e.g., a substituted or unsubstituted C₁₋₃₀ alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
   L⁴ is a bivalent polyamine or polyamide linker, e.g., a group of the formula:

—(CO)ᵣ—(CH₂)ₛ—(NR¹⁰C₂₋₆alkyl)ₜ-(NR¹¹)ᵤ—(CO)ᵥ—; or

—(CO)ᵣ—(CH₂)ₛ—(NR¹⁰C(O)—C₁₋₆ alkyl)ₜ-(NR¹¹)ᵤ—(CO)ᵥ—;
wherein:
R¹⁰ is hydrogen or C₁₋₃ alkyl;
R¹¹ is hydrogen or C₁₋₃ alkyl;
r is 0 or 1;
s is 0-10;
t is 1-10;
u is 0 or 1;
v is 0 or 1;
or R^{B} is -L⁵-IN; and E¹ is E² and/or E³ is —CH₃;
   wherein:
   L⁵ is a bivalent linker (e.g., a substituted or unsubstituted C₁₋₃₀ alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
   E² is —CH₂C(O)OR¹ or —CH₂CH₂C(O)OR¹;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl);
or a pharmaceutically acceptable salt thereof, or a stereoisomer or mixture of stereoisomers thereof.

In some embodiments, E² is —CH₂C(O)OR¹ or —CH₂CH₂C(O)OR¹. In some embodiments, E² is —CH₂C(O)OR¹. In some embodiments, E² is —CH₂CH₂C(O)OR¹.

In order to assure efficient release of the free drug, it is optionally also possible to incorporate what are called self-immolative linker elements (SIL) between the enzymatic cleavage site and drug (Anticancer Agents in Medicinal Chemistry, 2008, 8, 618-637). The drug can be released by various mechanisms, for example after initial enzymatic release of a nucleophilic group by subsequent elimination via an electronic cascade (Bioorg. Med. Chem., 1999, 7, 1597; J. Med. Chem., 2002, 45, 937; Bioorg. Med. Chem., 2002, 10,71) or by cyclization of the corresponding linker element (Bioorg. Med. Chem., 2003, 11, 2277; Bioorg. Med. Chem., 2007, 15, 4973; Bioorg. Med. Chem. Lett., 2007, 17, 2241) or by a combination of the two (Angew. Chem. Inter. Ed., 2005, 44, 4378). Examples of such linker elements are shown in Scheme 1:

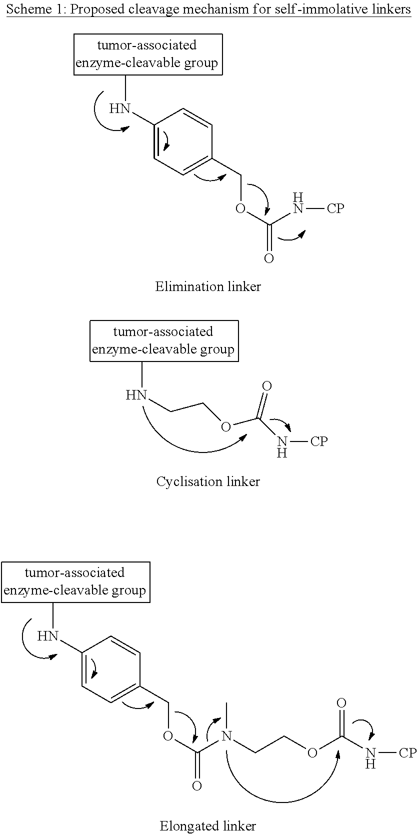

In some embodiments, SIL is a para-amino carbamate (PABC) group. In some embodiments, SIL is:

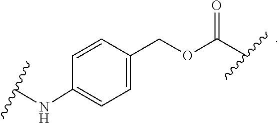 [SMILES: *Nc1ccc(COC(*)=O)cc1]

In some embodiments, SIL is absent (i.e., SIL is a bond).

In some embodiments, provided herein are compounds having a structure of Formula (I-A) or Formula (I-A′):

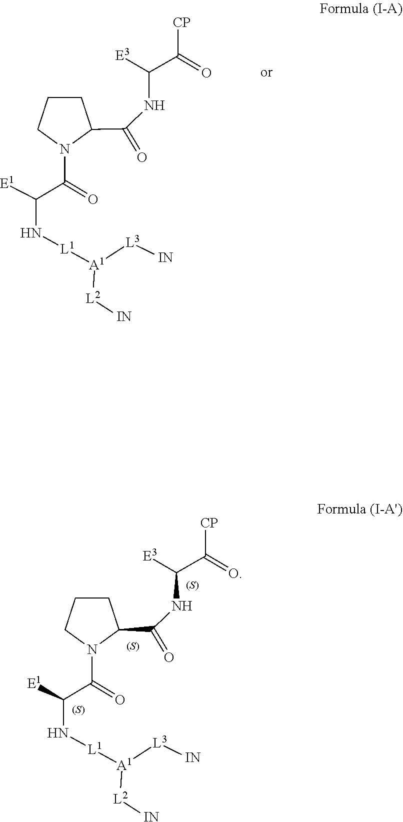

Formula (I-A) Formula (I-A′).

In some embodiments, provided herein are compounds having a structure of Formula (I-A) or Formula (I-A′):

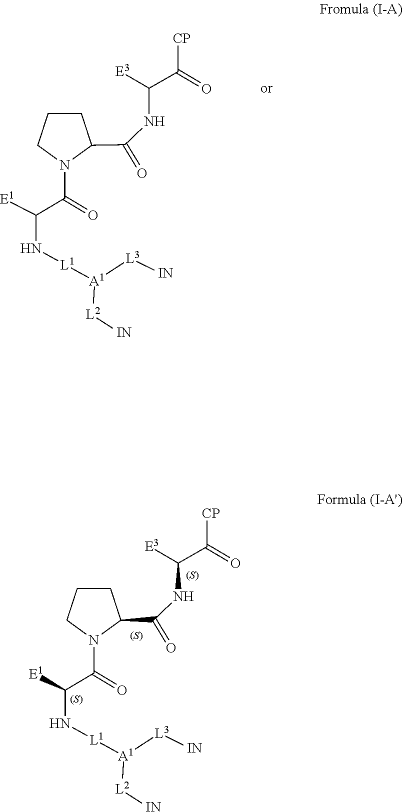

wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
IN is in each instance, independently, a monovalent radical of an integrin binder;
A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl));
each of L¹, L², and L³ is independently a bivalent linker (e.g., a substituted or unsubstituted C₁₋₃₀ alkyl, or substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker); and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl);
or a pharmaceutically acceptable salt thereof, or a stereoisomer or mixture of stereoisomers thereof.

In some embodiments, each of L¹, L², and L³ is a simple spacer, (as defined herein), or a polymeric spacer (as defined herein).

In some embodiments, provided herein are compounds having a structure of Formula (I-B) or Formula (I-B′):

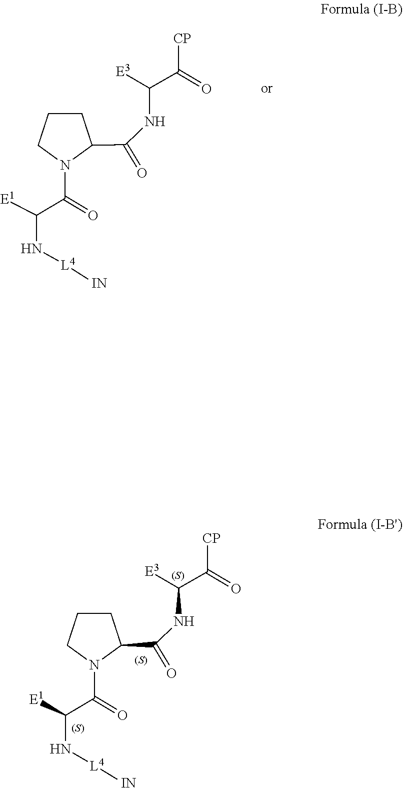

wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, or —CH₂C(O)OR¹;
E³ is —CH₃, —CH(CH₃)₂, —CH₂CH(CH₃)₂, or —CH(CH₃)CH₂CH₃;
IN is an integrin binder; and
L⁴ is a bivalent polyamine or polyamide linker (as defined herein).

In some embodiments, provided herein is a compound having a structure of Formula (I-B) or Formula (I-B′):

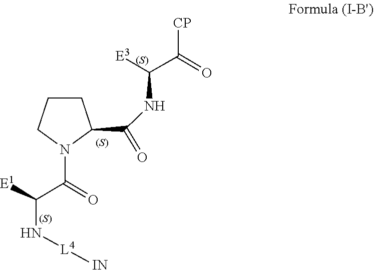

wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, or —CH₂C(O)OR¹;
E³ is —CH₃, —CH(CH₃)₂, —CH₂CH(CH₃)₂, or —CH(CH₃)CH₂CH₃;
IN is an integrin binder;
L⁴ is a group of the formula:

—(CO)ᵣ—(CH₂)ₛ—(NR¹⁰—C₁₋₆ alkyl)ₜ-(NR¹¹)ᵤ—(CO)ᵥ—; or

—(CO)ᵣ—(CH₂)ₛ—(NR¹⁰C(O)—C₁₋₆ alkyl)ₜ-(NR¹)ᵤ—(CO)ᵥ—;
wherein:
R¹⁰ is hydrogen or C₁₋₃ alkyl;
R¹¹ is hydrogen or C₁₋₃ alkyl;
r is 0 or 1;
s is 0-10;
t is 1-10;
u is 0 or 1;
v is 0 or 1;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl);
or a pharmaceutically acceptable salt thereof, or a stereoisomer or mixture of stereoisomers thereof.

In some embodiments, provided herein are compounds having a structure of Formula (I-B1) or Formula (I-B2):

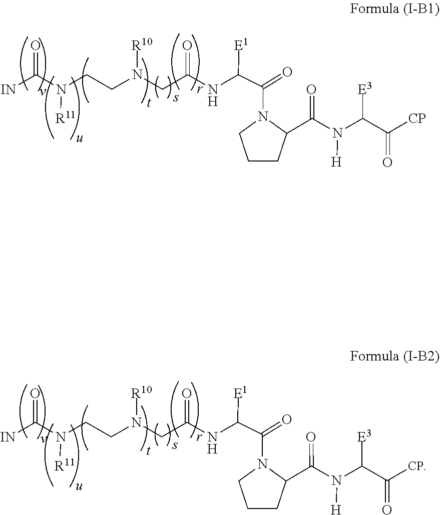

wherein:
CP is a cytotoxic or cytostatic group;
IN is a monovalent radical of an integrin binder;
E¹ is —H, —CH₃, —CH₂CH₃, —CH₂CH₂CH₃; —CH₂(heteroaryl), —CH₂C(O)NH₂, —CH₂C(O)OH, or —CH₂C(O)OR¹;
E³ is —CH₃, —CH(CH₃)₂, —CH₂CH(CH₃)₂, or —CH(CH₃)CH₂CH₃;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl);
R¹⁰ is hydrogen or C₁₋₃ alkyl;
R¹¹ is hydrogen or C₁₋₃ alkyl;
r is 0 or 1;
s is 0-10;
t is 1-10;
u is 0 or 1;
v is 0 or 1;
   or a pharmaceutically acceptable salt thereof, or a stereoisomer or mixture of stereoisomers thereof.

In some embodiments, a compound of Formula (I-B2) has the structure:

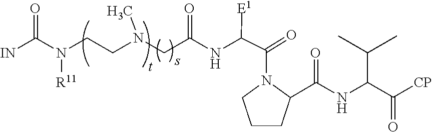

or a pharmaceutically acceptable salt thereof, or a stereoisomer or mixture of stereoisomers thereof. In some embodiments, s is 1. In some embodiments, s is 2. In some embodiments, t is 1. In some embodiments, t is 2. In some embodiments, t is 3. In some embodiments, t is 4. In some embodiments, t is 5. In some embodiments, t is 6. In some embodiments, s is 1 and t is 2. In some embodiments, s is 2 and t is 2. In some embodiments, s is 1 and t is 3. In some embodiments, s is 2 and t is 3. In some embodiments, s is 1, t is 2 or 3, and R¹¹ is hydrogen or —CH₃. In some embodiments, s is 1, t is 2, and R¹¹ is hydrogen. In some embodiments, s is 1, t is 3, and R¹¹ is —CH₃.

In some embodiments, provided herein are compounds having a structure of Formula (I-C) or Formula (I-C′):

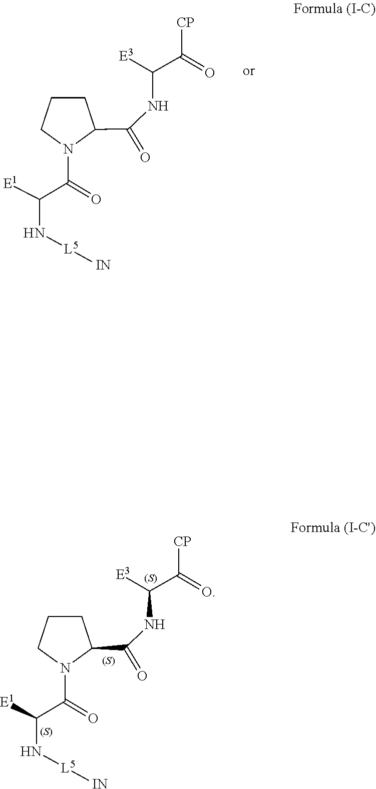

In some embodiments, provided herein are compounds having a structure of Formula (I-C) or Formula (I-C′):

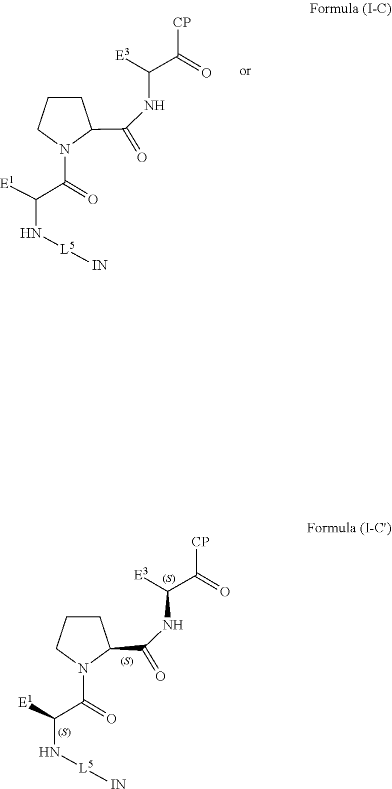

wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹; or E¹ is E²
E² is —CH₂C(O)OR¹ or —CH₂CH₂C(O)OR¹;
E³ is —CH₃, —CH(CH₃)₂, —CH₂CH(CH₃)₂, or —CH(CH₃)CH₂CH₃;
IN is an integrin binder;
L⁵ is a bivalent linker (e.g., a substituted or unsubstituted alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein; L⁶ is a bivalent linker (e.g., substituted or unsubstituted C₁₋₃₀ alkyl, or substituted or unsubstituted heteroalkyl);
or a pharmaceutically acceptable salt thereof, or a stereoisomer or mixture of stereoisomers thereof.

In some embodiments, E³ is —CH₃ and E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹. In some embodiments, E³ is —CH(CH₃)₂, and E¹ is E², wherein E² is —CH₂C(O)OR¹ or —CH₂CH₂C(O)OR¹; and R¹ is as defined above.

In some embodiments, provided herein are compounds having a structure of Formula (I-C1):

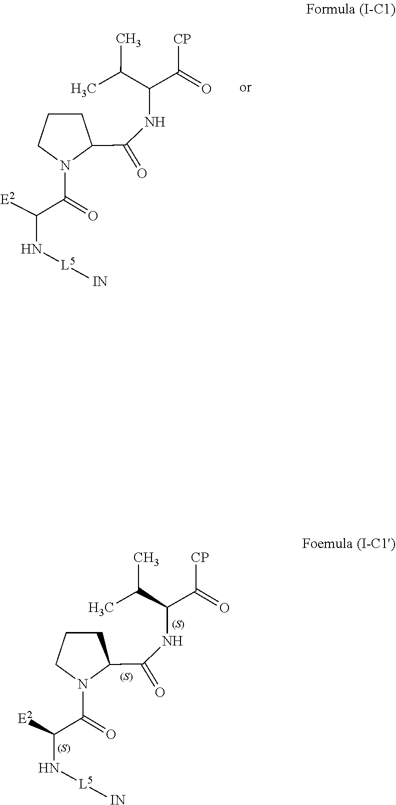

wherein:
CP is a cytotoxic or cytostatic group;
E² is hydrogen, —CH₃, —CH₂CH₃, —CH₂CH₂CH₃, —CH₂C(O)NH₂, —CH₂C(O)OR¹, or —CH₂CH₂C(O)OR¹;
IN is an integrin binder;
L⁵ is a bivalent linker (e.g., a substituted or unsubstituted alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein; L⁶ is a bivalent linker (e.g., substituted or unsubstituted C₁₋₃₀ alkyl, or substituted or unsubstituted heteroalkyl);
or a pharmaceutically acceptable salt thereof, or a stereoisomer or mixture of stereoisomers thereof.

In some embodiments, provided herein are compounds having a structure of Formula (I-C2) or Formula (I-C2′):

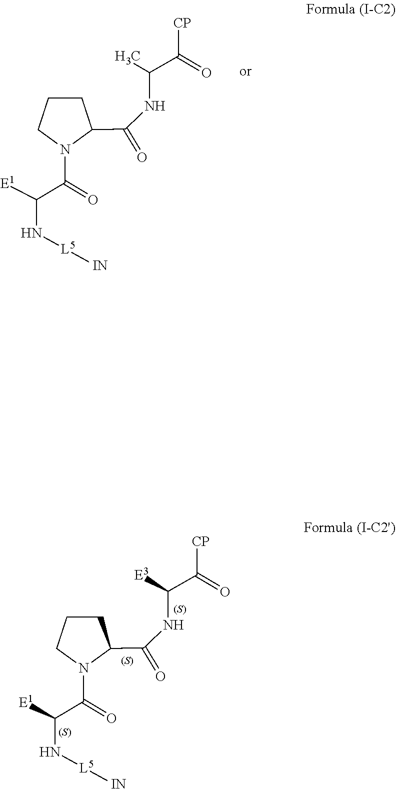

wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹;
IN is an integrin binder;
L⁵ is a bivalent linker (e.g., a substituted or unsubstituted alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein; L⁶ is a bivalent linker (e.g, substituted or unsubstituted C₁₋₃₀ alkyl, or substituted or unsubstituted heteroalkyl);
or a pharmaceutically acceptable salt thereof, or a stereoisomer or mixture of stereoisomers thereof.

In some embodiments, provided herein is a compound having a structure of Formula (I-C), Formula (I-C1), or Formula (I-C2), wherein:
CP is a cytotoxic or cytostatic group (e.g., a 7-ethyl camptothecin group);
L⁵ is a bivalent linker (e.g., a substituted or unsubstituted alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker);
L⁶ is a substituted or unsubstituted alkyl linker, or a substituted or unsubstituted heteroalkyl linker;
MOD is a physicochemical or pharmacokinetic modulating group; and
IN is an integrin binding group.

In some embodiments, provided herein is a compound having a structure of Formula (I-C), Formula (I-C1), or Formula (I-C2), wherein:
L⁵ is a linker having a structure represented by the formula:

—(CO)ₘ—(CH₂)ₙ—(OC₂₋₆ alkyl)ₒ-(NH)ₚ—(CO)_{q}—; (i)

—(CO)ᵣ—(CH₂)ₛ—(NR¹⁰C₂₋₆ alkyl)ₜ-(NR¹¹)ᵤ—(CO)ᵥ—; or (ii)

—(CO)ᵣ—(CH₂)ₛ—(NR¹⁰C(O)C₁₋₆ alkyl)ₜ-(NR¹¹)ᵤ—(CO)ᵥ—; (iii)
wherein:
each R¹⁰ and R¹¹ is independently hydrogen or C₁₋₃ alkyl;
each m, p, q, r, u, and v is independently 0 or 1; and
each n, o, s, and t is independently 0, 1, 2, 3, 4, 5, 6, 7, 8, 9, or 10.

In some embodiments, the compound has the structure:

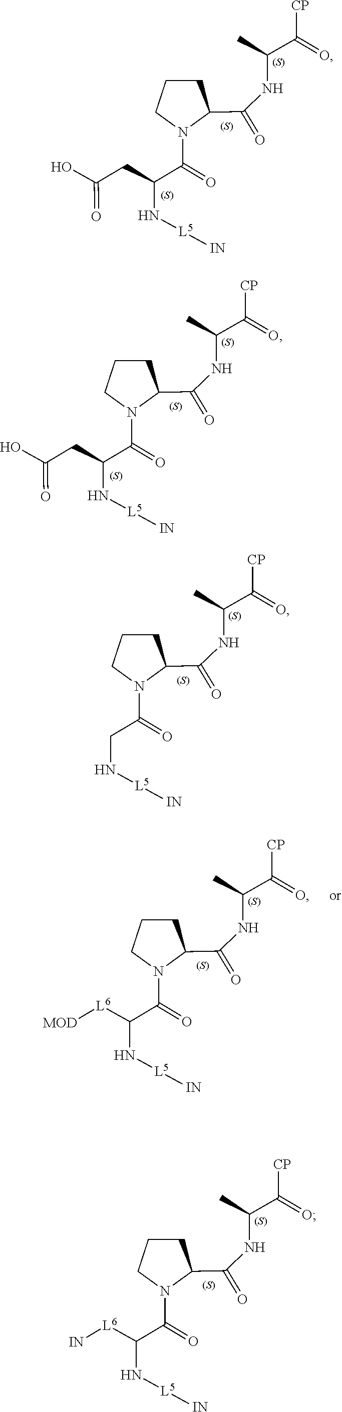

wherein:
CP is a cytotoxic or cytostatic group (e.g., a 7-ethyl camptothecin group);
L⁵ and L⁶ are each independently substituted or unsubstituted alkyl, or substituted or unsubstituted heteroalkyl linkers
MOD is a physicochemical or pharmacokinetic modulating group; and
IN is an integrin binding group.

In some embodiments, L⁵ is —(CO)ₘ—(CH₂)ₙ—(OC₂₋₆ alkyl)ₒ-(NH)ₚ—(CO)_{q}—. In some embodiments, L⁵ is —C(O)—C₂₋₆ alkyl-[O—C₂₋₆ alkyl]₁₋₈-NHC(O)—. In some embodiments, L⁵ is —C(O)—C₁₋₆ alkyl-[N(CH₃)—C₂₋₆ alkyl]₁₋₈-NHC(O)—. In some embodiments, L⁵ is —C(O)—C₁₋₆ alkyl-[N(CH₃)—C₂₋₆ alkyl]₁₋₈-N(CH₃)C(O)—. In some embodiments, L⁵ is —C(O)—C₁₋₆ alkyl-[N(CH₃)C(O)—C₁₋₆ alkyl]₁₋₈-N(CH₃)—C(O)—. In some embodiments, L⁵ is —C(O)—CH₂CH₂—[OCH₂CH₂]₃—NHC(O)—. In some embodiments, L⁵ is —C(O)—CH₂—[N(CH₃)CH₂CH₂]₂—N(CH₃)C(O)—. In some embodiments, L⁵ is —C(O)—CH₂—[N(CH₃)CH₂CH₂]₃—NHC(O)—. In some embodiments, L⁵ is —C(O)—CH₂—[N(CH₃)C(O)CH₂]₃—N(CH₃)C(O)—.

In some embodiments, E² is —CH₂C(O)OR¹ or —CH₂CH₂C(O)OR¹. In some embodiments, E² is —CH₂C(O)OR¹. In some embodiments, E² is —CH₂CH₂C(O)OR¹.

In some embodiments, provided herein are compounds having a structure of Formula (I-C1), Formula (I-C2), or Formula (I-C3):

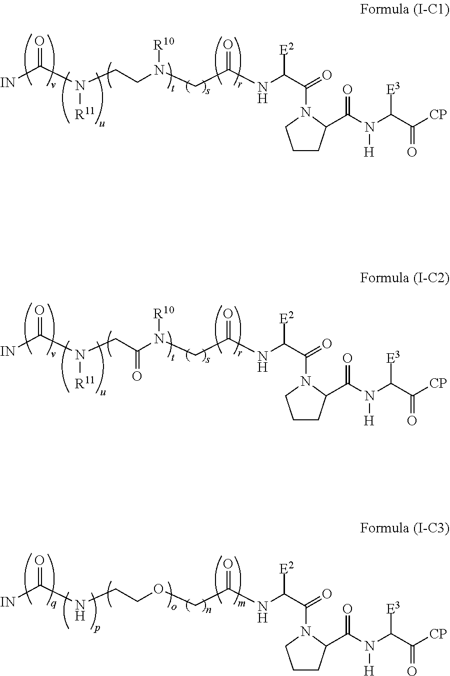

wherein:
CP is a cytotoxic or cytostatic group;
IN is a monovalent radical of an integrin binder;
E² is —CH₂C(O)OR¹;
E³ is —CH₃, —CH(CH₃)₂, —CH₂CH(CH₃)₂, or —CH(CH₃)CH₂CH₃; and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl;
R¹⁰ is hydrogen or C₁₋₃ alkyl;
R¹¹ is hydrogen or C₁₋₃ alkyl;
r is 0 or 1;
s is 0-10;
t is 1-10;
u is 0 or 1;
v is 0 or 1;
   or a pharmaceutically acceptable salt thereof, or a stereoisomer or mixture of stereoisomers thereof.

In some embodiments, the compound has the structure:

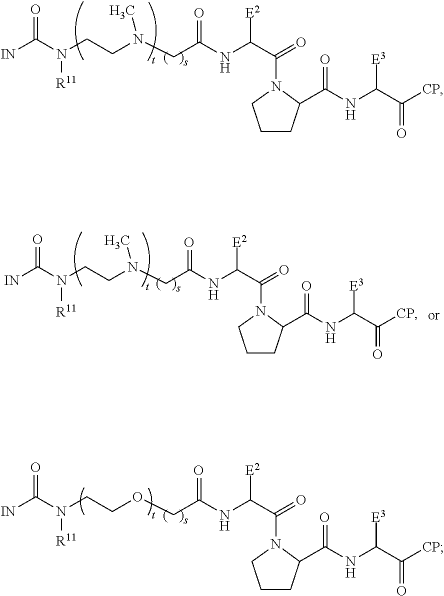

or a pharmaceutically acceptable salt thereof, or a stereoisomer or mixture of stereoisomers thereof. In some embodiments, s is 1. In some embodiments, s is 2. In some embodiments, t is 1. In some embodiments, t is 2. In some embodiments, t is 3. In some embodiments, t is 4. In some embodiments, t is 5. In some embodiments, t is 6. In some embodiments, s is 1 and t is 2. In some embodiments, s is 2 and t is 2. In some embodiments, s is 1 and t is 3. In some embodiments, s is 2 and t is 3. In some embodiments, s is 1, t is 2 or 3, and R¹¹ is hydrogen or -CH₃. In some embodiments, s is 1, t is 2, and R¹¹ is hydrogen. In some embodiments, s is 1, t is 3, and R¹¹ is —CH₃.

In some embodiments, provided herein are compounds having a structure of Formula (I-D) or Formula (I-D′):

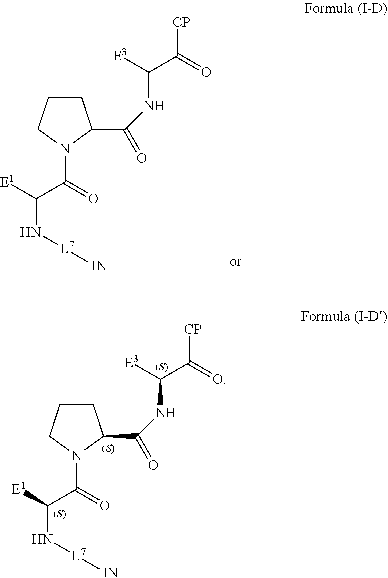

In some embodiments, provided herein are compounds having a structure of Formula (I-D) or Formula (I-D′):

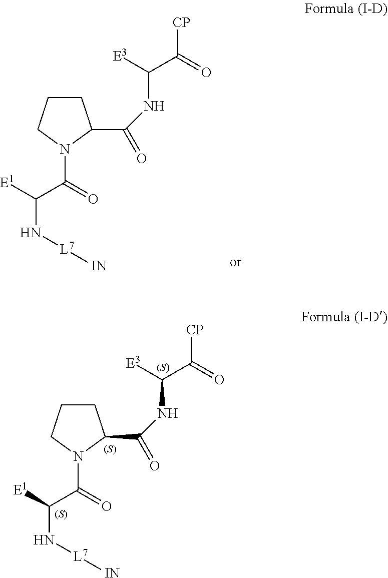

wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
IN is a monovalent radical of an integrin binder;
L⁷ is a substituted or unsubstituted C₁₋₆₀ alkyl, or substituted or unsubstituted heteroalkyl comprising 4-12 heteroalkyl units, wherein each Z group is independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, or —NHS(O)₂NHC(O)—;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆ alkyl, —CN, —CONH₂, —CONH(C₁₋₆ alkyl), —CON(C₁₋₆alkyl)₂, —COOH, —COO(C₁₋₆ alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl);
   or a pharmaceutically acceptable salt thereof, or a stereoisomer or mixture of stereoisomers thereof.

In some embodiments, provided herein are compounds having a structure of Formula (I-E) or Formula (I-E′):

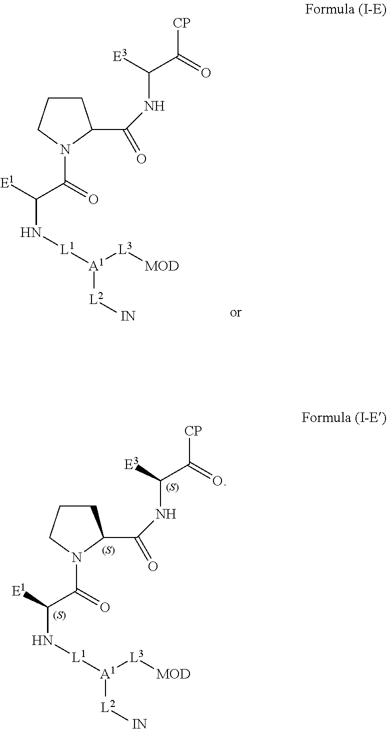

In some embodiments, provided herein are compounds having a structure of Formula (I-E) or Formula (I-E′):

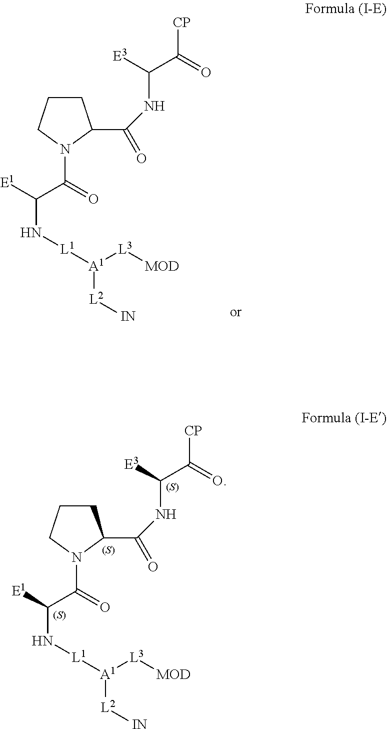

or a pharmaceutically acceptable salt thereof, or a stereoisomer or mixture of stereoisomers thereof, wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, —CH₂CH₂C(O)OH, —CH₂CH₂C(O)OR¹;
E³ is —CH₃, —CH(CH₃)₂, —CH₂CH(CH₃)₂, or —CH(CH₃)CH₂CH₃;
A¹ is a trivalent linker(e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl, or heteroalkyl-heteroaryl));
each of L¹, L², and L³ is independently a bivalent linker (e.g., a substituted or unsubstituted C₁₋₃₀ alkyl, or substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker); and
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆ alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆ alkyl), —SO₂NH₂, —SO₂NH(C₁₋₆ alkyl), and —SO₂N(C₁₋₆alkyl)₂; wherein L⁶ is a bivalent linker (e.g., substituted or unsubstituted C₁₋₃₀ alkyl, or substituted or unsubstituted heteroalkyl);
IN is an integrin binding group; and
MOD is a physicochemical or pharmacokinetic modulating group.

In some embodiments, MOD is —COOH, —COONa—COOCH₃, —NH₂, —N(CH₃)₂, —N(CH₃)₃, —NC(═NH)NH₂, —OH, or —OCH₃. In some embodiments, MOD is —COOH, —NH₂, —N(CH₃)₂, —N(CH₃)₃⁺, —N(═NH)NH₂, or —OH. In some embodiments, MOD is an anion-forming physicochemical or pharmacokinetic modulator. In some embodiments, MOD is —COOH or —OH (i.e., —COO⁻ or —O⁻, or a salt thereof). In some embodiments, MOD is —COOH, or a salt thereof. In some embodiments, MOD is —OH, or a salt thereof. In some embodiments, MOD is a cation-forming physicochemical modulator. In some embodiments, MOD is —NH₂, —N(CH₃)₂, or —N(CH₃)₃⁺. In some embodiments, MOD is a physicochemical modulator comprising a polar amino acid, or a derivative thereof. In some embodiments, MOD is or comprises a polyamine or polyamide. In some embodiments, MOD is or comprises a polypeptide (e.g., a natural polypeptide or an unnatural polypeptide).

In some embodiments, CP is a 7-ethyl camptothecin group. In some embodiments, CP is:

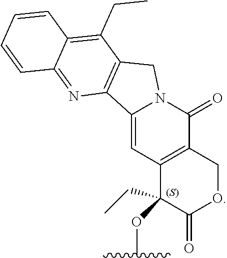

In some embodiments, provided herein is a structure of Formula (II) or Formula (II′):

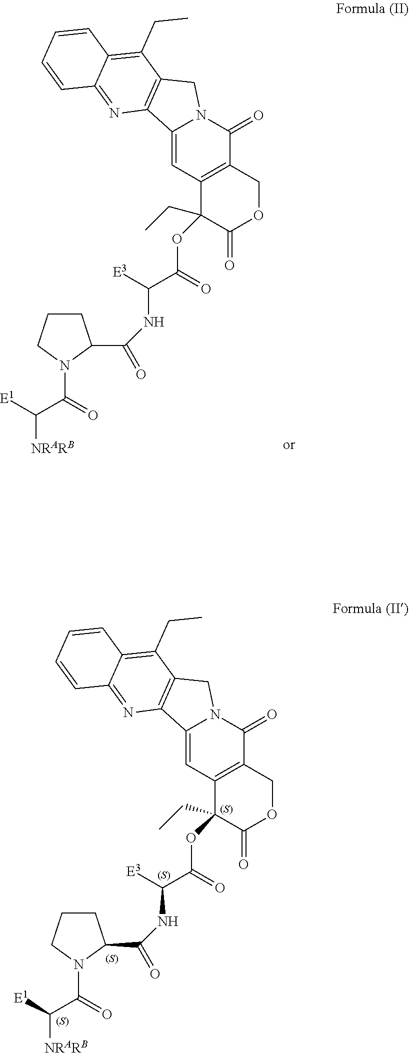

or a pharmaceutically acceptable salt thereof, or a stereoisomer or mixture of stereoisomers thereof.

In some embodiments, a compound of Formula (II) has a structure represented by Formula (II-A), Formula (II-B), Formula (II-C), Formula (II-D), or Formula (II-E):

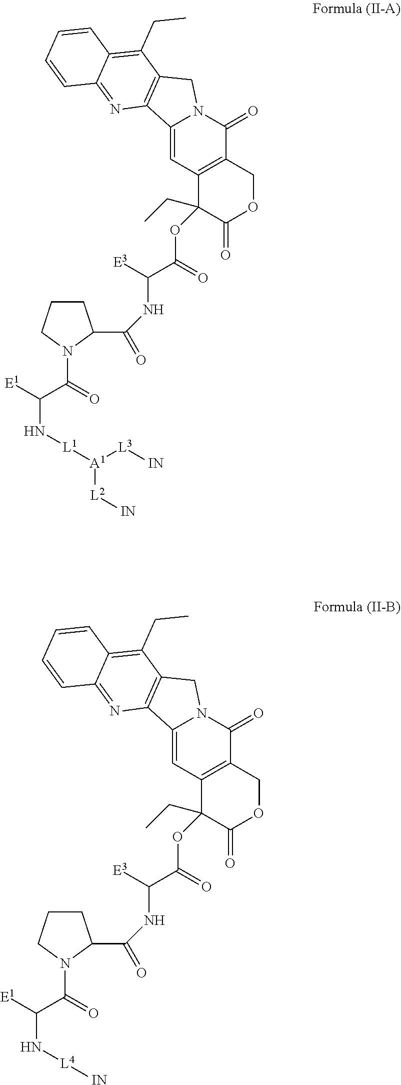

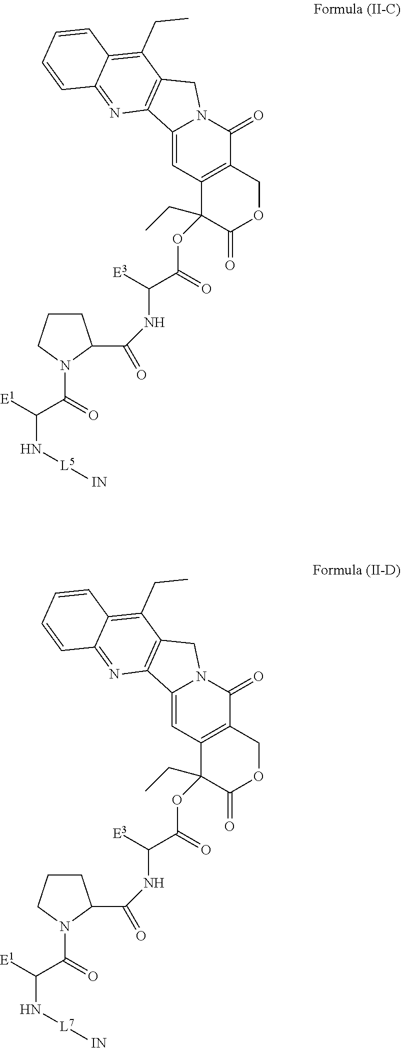

or a pharmaceutically acceptable salt thereof, or a stereoisomer or mixture of stereoisomers thereof.

In some embodiments, a compound of Formula (II′) has a structure represented by Formula (II-A′), Formula (II-B′), Formula (II-C′), Formula (II-D′), or Formula (II-E′):

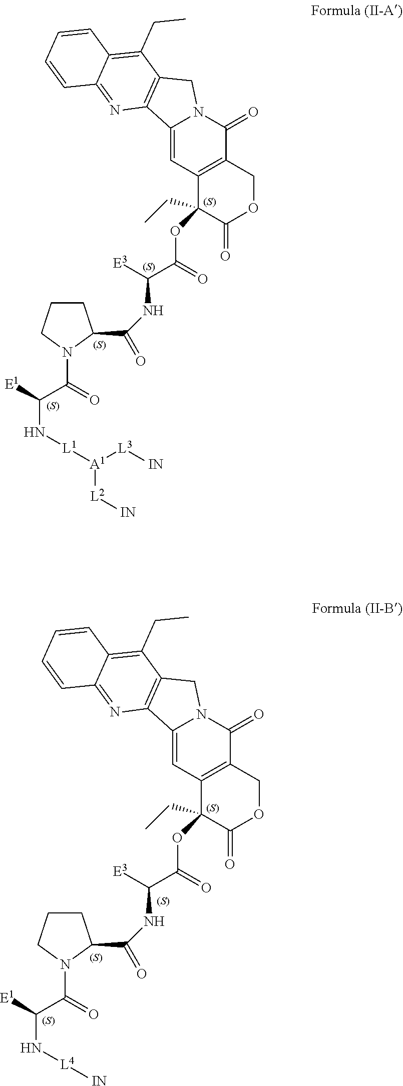

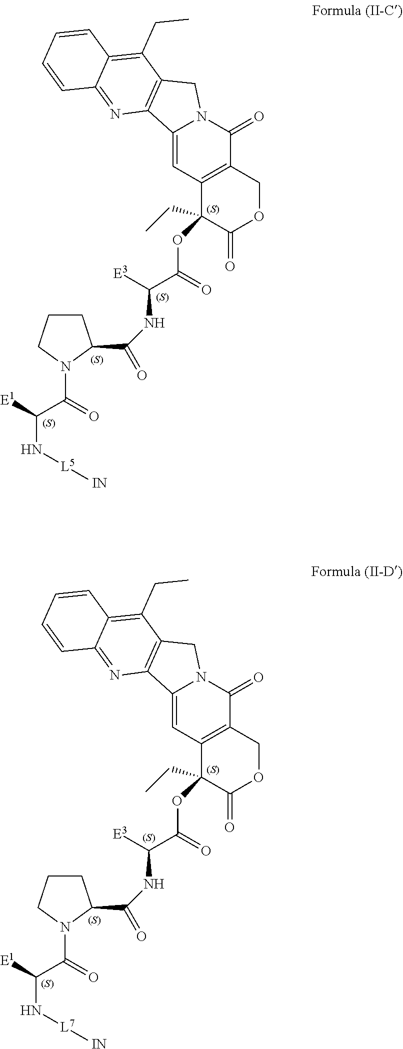

or a pharmaceutically acceptable salt thereof, or a stereoisomer or mixture of stereoisomers thereof.

In some embodiments, E³ is —CH₃ or —CH(CH₃)₂. In some embodiments, E³ is —CH₃. In some embodiments, E³ is —CH(CH₃)₂. In some embodiments, E¹ is hydrogen, —CH₂C(O)NH₂, —CH₂C(O)OH, or —CH₂C(O)OR, wherein R is a substituted or unsubstituted alkyl group, or R is a linker to an integrin binder (IN) or a physicochemical or PK modulator (MOD). In some embodiments, E¹ is —CH₂COOH. In some embodiments, E¹ is —CH₂CONH₂. In some embodiments, E¹ is hydrogen. In some embodiments, E¹ is hydrogen, —CH₃, —CH₂CH₃, —CH₂CH₂CH₃, —CH₂C(O)NH₂, —CH₂C(O)OH, —CH₂C(O)OR¹.

In some embodiments, a compound of Formula (II) has a structure represented by Formula (II-E) or Formula (II-E′):

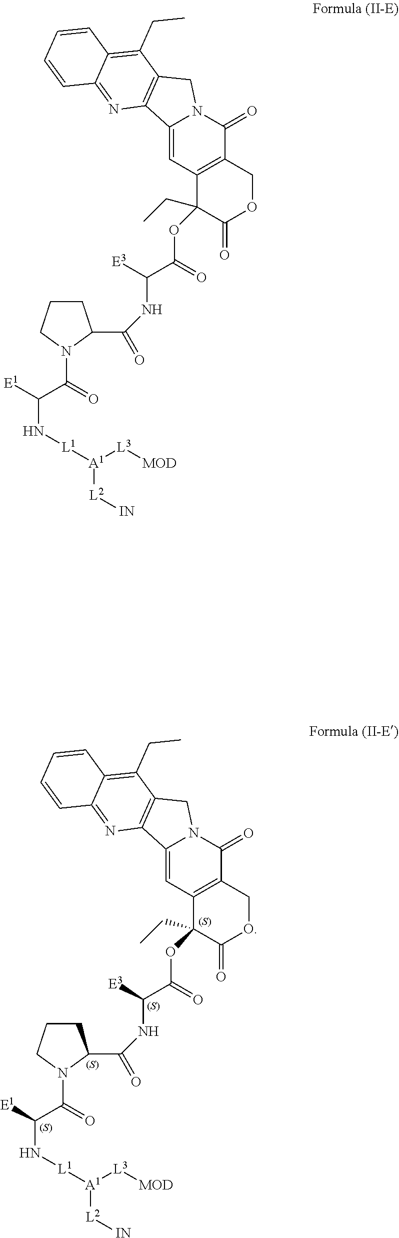

wherein each of E¹, E³, L¹, L², L³, A¹, IN, and MOD are as defined in Formula (I-E).

In some embodiments, the present invention provides compounds having a structure of formulae disclosed herein, or a pharmaceutically acceptable salt thereof, or a stereoisomer or mixture of stereoisomers thereof; wherein IN is a peptidic or peptidomimetic integrin binder.

In some embodiments, the present invention provides compounds having a structure of formulae disclosed herein, or a pharmaceutically acceptable salt thereof, or a stereoisomer or mixture of stereoisomers thereof, wherein IN is an integrin binder comprising non-peptides, non-antibodies, or small molecules. In some embodiments, IN is a small molecule integrin binder. In some embodiments, IN is a small molecule αᵥβ₃ integrin binder. In some embodiments, each IN is a small molecule αᵥβ₃ integrin binder. In some embodiments, IN is a macrocyclic integrin binding peptide. In some embodiments, the linear integrin binding peptide is a constrained macrocyclic integrin binding peptide. In some embodiments, IN is a non-peptidic or non-peptidomimetic integrin binder.

In some embodiments, IN is a tumor-binding moiety. In some embodiments, IN binds to both tumor and non-tumor cells. In some embodiments, IN binds rapidly dividing cells (e.g., tumor cells or other hyperproliferating cells).

In some embodiments, IN has the structure:

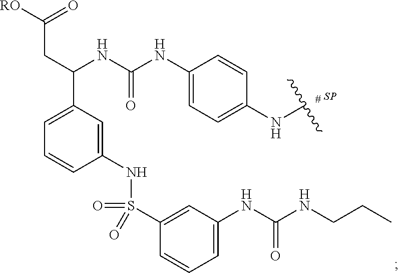

or a pharmaceutically acceptable salt thereof; or a stereoisomer (e.g., IN-2a, IN-2b); wherein #^{SP} denotes a bond to one of L², L³, L⁴, L⁵, L⁶, or L⁷; and R is hydrogen or substituted or unsubstituted alkyl; wherein if R is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), -MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂. In some embodiments, R is a substituted or unsubstituted C₁₋₁₂ alkylamine. In some embodiments, R is hydrogen. In some embodiments, IN is:

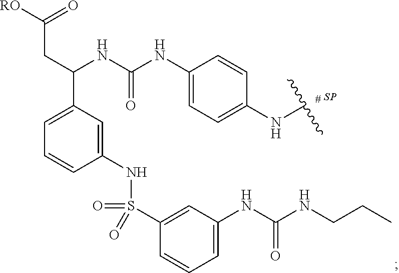

or a pharmaceutically acceptable salt thereof; or a stereoisomer thereof (e.g., IN-1a, IN-1b)

In some embodiments, provided herein is a compound of Formula (III-A), Formula (III-B), Formula (III-C), Formula (III-D), or Formula (III-E):

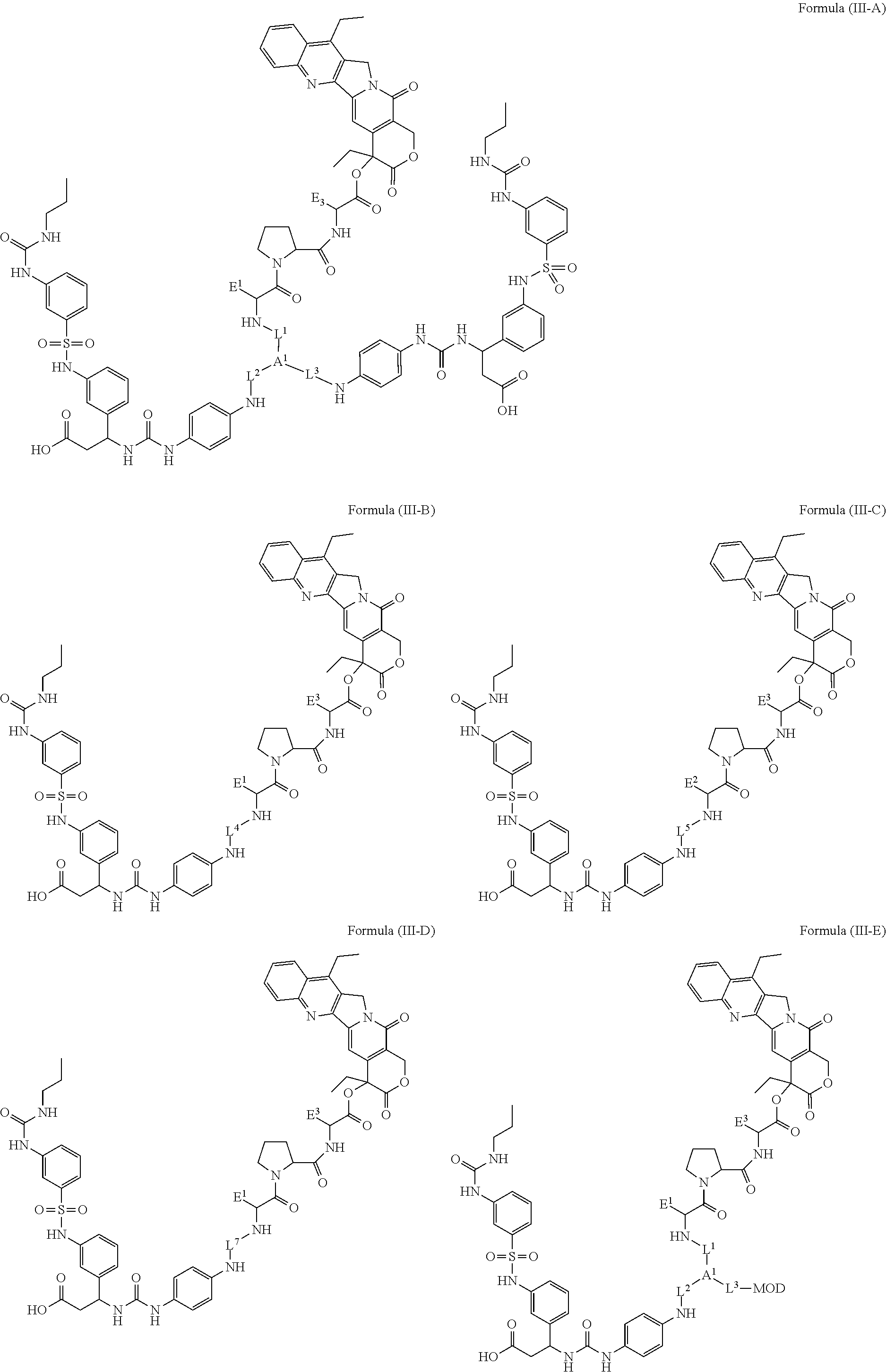

or a pharmaceutically acceptable salt thereof, or a stereoisomer or mixture of stereoisomers thereof.

In some embodiments, E³ is —CH₃ or —CH(CH₃)₂. In some embodiments, E³ is —CH₃. In some embodiments, E³ is —CH(CH₃)₂. In some embodiments, E¹ is hydrogen, —CH₂C(O)NH₂, —CH₂C(O)OH, or —CH₂C(O)OR, wherein R is a substituted or unsubstituted alkyl group, or R is a linker to an integrin binder (IN) or a physicochemical or pharmacokinetic modulator (MOD). In some embodiments, E¹ is —CH₂COOH. In some embodiments, E¹ is —CH₂CONH₂. In some embodiments, E¹ is hydrogen. In some embodiments, E¹ is hydrogen, —CH₃, —CH₂CH₃, —CH₂CH₂CH₃, —CH₂C(O)NH₂, —CH₂C(O)OH, —CH₂C(O)OR¹.

In some embodiments, provided herein is a compound of Formula (III-A′), Formula (III-B′), Formula (III-C′), Formula (III-D′), or Formula (III-E′):

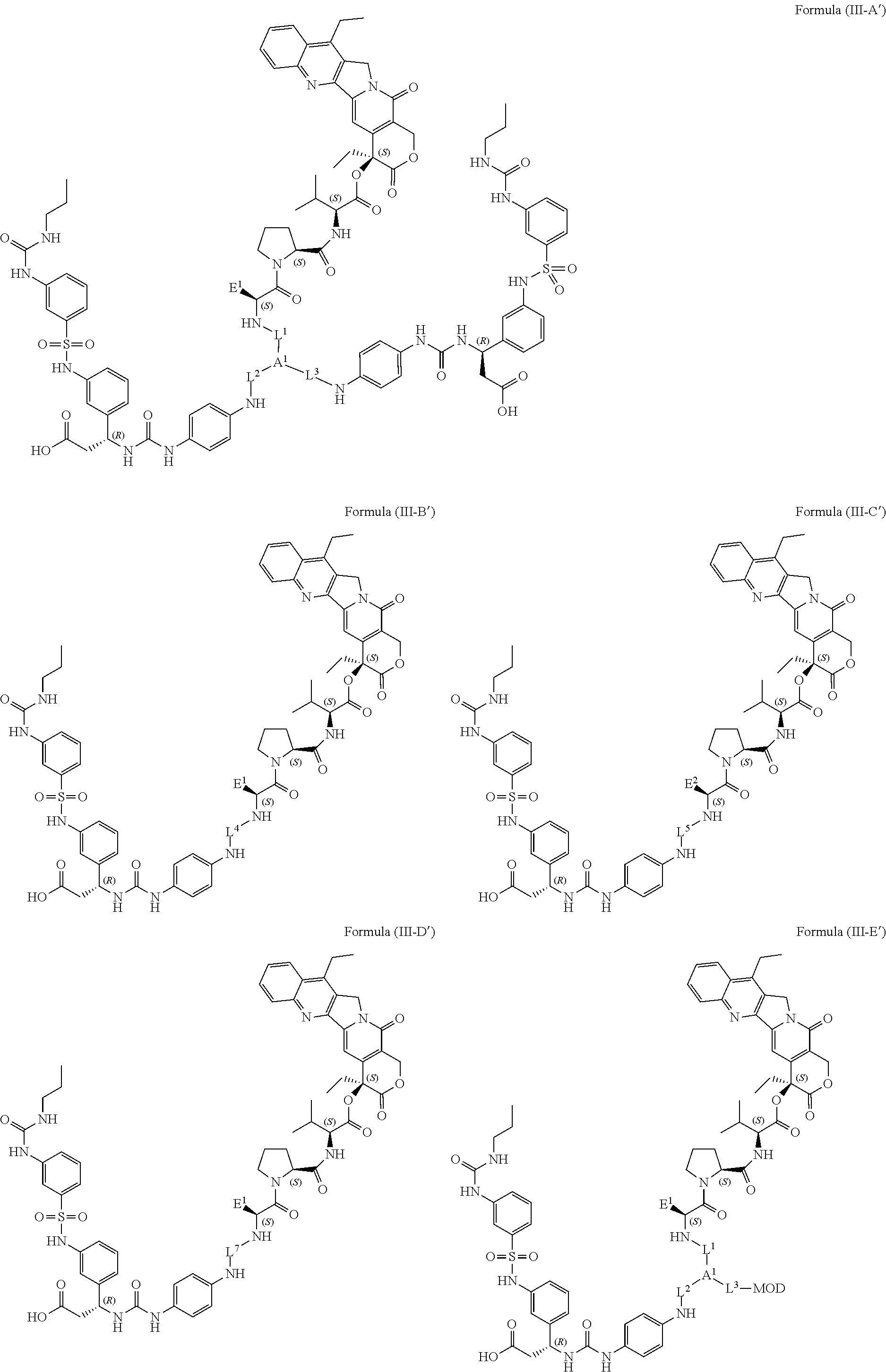

or a pharmaceutically acceptable salt thereof, or a stereoisomer or mixture of stereoisomers thereof.

In some embodiments, provided herein is a compound, or a pharmaceutically acceptable salt thereof, or a stereoisomer or mixture of stereoisomers thereof, of Formula (III-A):

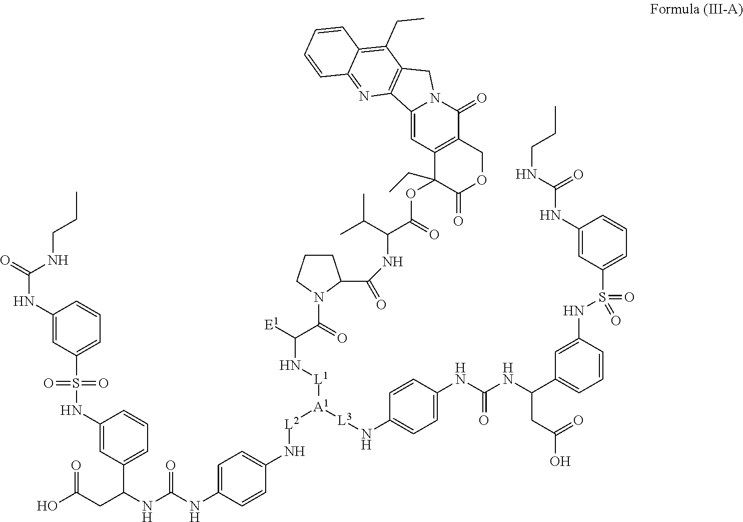

wherein:
each of L¹, L², and L³ is independently a bivalent linker (e.g., a substituted or unsubstituted C₁₋₃₀ alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker); and
A¹ is a trivalent linker (e.g., a substituted or unsubstituted trivalent radical selected from the group consisting of alkyl, heteroalkyl, cycloalkyl, heterocyclyl, aryl, heteroaryl, or a combination thereof (e.g., aralkyl, heteroalkyl-aryl, alkyl-heteroaryl heteroalkyl-heteroaryl)).

In some embodiments, each of L¹, L², and L³ is independently a simple spacer or a polymeric spacer (as defined herein). In some embodiments, each of L¹, L², and L³ is independently selected from the list of simple spacers (as defined herein). In some embodiments, each of L¹, L², and L³ is independently a simple spacer selected from —C(O)—, —C(O)—C₁₋₃₀ alkyl-, —C(O)—C₁₋₃₀ alkyl-C(O)—, —C(O)—C₁₋₃₀ alkyl-C(O)NH—, —C(O)—C₁₋₃₀ alkyl-C(O)N(CH₃)—, —C(O)—C₁₋₃₀ alkyl-NH—, —C(O)—C₁₋₃₀ alkyl-NHC(O)—, —C(O)—C₁₋₃₀ alkyl-N(CH₃)—, or —C(O)—C₁₋₃₀ alkyl-N(CH₃)C(O)—.

In some embodiments, each of L¹, L², and L³, is a polymeric spacer having a structure represented by formula (i), formula (ii), or formula (iii) below:

—(CO)ₘ(CH₂)ₙ(OC₂₋₆ alkyl)ₒ(NH)ₚ(CO)_{q}—; (i)

—(CO)ᵣ(CH₂)ₛ(NR¹⁰C₁₋₆ alkyl)ₜ(NR¹¹)ᵤ(CO)ᵥ—; or (ii)

—(CO)ᵣ—(CH₂)ₛ—(NR¹⁰C(O)C₁₋₆ alkyl)ₜ-(NR¹¹)ᵤ—(CO)ᵥ—; (iii)
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1; n is 0 to 10; o is 1 to 10; p is 0 or 1; and q is 0 or 1; and r is 0 or 1; s is 0 to 10; t is 1 to 10; u is 0 or 1; v is 0 or 1.

In some embodiments, L¹ is a polymeric spacer and L² and L³ are each simple or polymeric spacers. In some embodiments, each of L¹, L², and L³, is a polymeric spacer. In some embodiments, one or more of L¹, L², and L³, is a polyamine or a polyamide spacer of formula (ii) or formula (iii).

In some embodiments, provided herein is a compound of Formula (III-A) wherein:
E¹ is hydrogen, —CH₂C(O)NH₂, —CH₂C(O)OH, —CH₂C(O)OR¹;
R¹ is C₁₋₁₂ alkyl substituted with —NHL⁶-IN, —N(C₁₋₆alkyl)₂, or —N(C₁₋₆alkyl)₃⁺;
L¹ is —C(O)—C₁₋₆ alkyl-[O—C₂₋₆ alkyl]₁₋₈-N*, —C(O)—C₁₋₆ alkyl-[N(CH₃)—C₂₋₆ alkyl]₁₋₈-NH—*, —C(O)—C₁₋₆ alkyl-[N(CH₃)—C₂₋₆ alkyl]₁₋₈-N(CH₃)—*;
A¹ is a trivalent amide linker;
L² is C(O)—,
   **C(O)—C₁₋₆ alkyl-NHC(O)—,
   **C(O)—C₁₋₆ alkyl-[O—C₂₋₆ alkyl]₁₋₈-NHC(O)—,
   **C(O)—C₁₋₆ alkyl-[N(CH₃)—C₂₋₆ alkyl]₁₋₈-NHC(O)—, or
   **C(O)—C₁₋₆ alkyl-[N(CH₃)—C₂₋₆ alkyl]₁₋₈-N(CH₃)C(O)—;
L³ is ***C(O)—,
   ***C(O)—C₁₋₆ alkyl-NHC(O)—,
   ***C(O)—C₁₋₆ alkyl-[O—C₂₋₆ alkyl]₁₋₈-NHC(O)—,
   ***C(O)—C₁₋₆ alkyl-[N(CH₃)—C₂₋₆ alkyl]₁₋₈-NHC(O)—, or
   ***C(O)—C₁₋₆ alkyl-[N(CH₃)—C₂₋₆ alkyl]₁₋₈-N(CH₃)C(O)—; and
L⁶ is —C(O)—;
   or a pharmaceutically acceptable salt thereof, or a stereoisomer or mixture of stereoisomers thereof.

In some embodiments, A¹ is a trivalent linker having the structure of Formula (A¹-1a), Formula (A¹-2a), Formula (A¹-3a), or Formula (A¹-4a). In some embodiments, A¹ is a trivalent linker having the structure of Formula (A¹-1b), Formula (A¹-2b), Formula (A¹-3b), or Formula (A¹-4b). In some embodiments, A¹ is a trivalent linker having the structure of Formula (A¹-1c), Formula (A¹-2c), Formula (A¹-3c), or Formula (A¹-4c). In some embodiments, A¹ is a trivalent linker having the structure of Formula (A¹-1d), Formula (A¹-2d), Formula (A¹-3d), or Formula (A¹-4d).

In some embodiments:
A¹ is *C(O)—C₀₋₆ alkyl-Y(C₀₋₆ alkyl-NH**)(C₀₋₆ alkyl-NH***),
   *C(O)—C₀₋₆ alkyl-Y(C₀₋₆ alkyl-NH**)(C₀₋₆ alkyl-C(O)***),
   *C(O)—C₀₋₆ alkyl-Y(C₀₋₆ alkyl-C(O)**)(C₀₋₆ alkyl-C(O)***),
   *C(O)—C₀₋₆ alkyl-C(O)NH—Y(C₀₋₆ alkyl-NH**)(C₀₋₆ alkyl-NH***),
   *C(O)—C₀₋₆ alkyl-C(O)NH—Y(C₀₋₆ alkyl-NH**)(C₀₋₆ alkyl-C(O)***),
   *C(O)—C₀₋₆ alkyl-C(O)NH—Y(C₀₋₆ alkyl-C(O)**)(C₀₋₆ alkyl-C(O)***),
   *C(O)—C₀₋₆ alkyl-C(O)NH—Y(C₀₋₆ alkyl-C(O)NH—C₀₋₆ alkyl-NH**)(C₀₋₆ alkyl-C(O)NH—C₀₋₆ alkyl-NH***);
   *C(O)—C₁₋₆ alkyl-C(O)NH—C₁₋₆ alkyl-Y(NH**)(C(O)***); or
   *C(O)—Y(NH**)(C₀₋₆ alkyl-NHC(O)—C₀₋₆ alkyl-C(O)***);
   wherein:
   Y is CH or N;
   * is a bond between L¹ and A¹;
   ** is a bond between A¹ and L²
   *** is a bond between A¹ and L³;
In some embodiments, provided herein is a compound of Formula (III-A), or a pharmaceutically acceptable salt thereof, or a stereoisomer or mixture of stereoisomers thereof, wherein:
E¹ is hydrogen, —CH₂C(O)NH₂ or —CH₂C(O)OH;
L¹ is —C(O)—C₂₋₄ alkyl-[O—C₂₋₄ alkyl]₂₋₄-NH*,
   —C(O)—C₁₋₄ alkyl-[N(CH₃)—C₂₋₄ alkyl]₂₋₄-NH—*,
   —C(O)—C₁₋₄ alkyl-[N(CH₃)—C₂₋₄ alkyl]₂₋₄-N(CH₃)—*;
A¹ is *C(O)—CH(NH**)(C₁₋₄ alkyl-NH***),
   *C(O)—C₁₋₄ alkyl-C(O)NH—CH(C₁₋₄ alkyl-C(O)NH—C₁₋₄ alkyl-NH**)(C(O)NH—C₁₋₄ alkyl-NH***),
   *C(O)—C₁₋₆ alkyl-C(O)NH—C₁₋₆ alkyl-CH(NH**)(C(O)***); or
   *C(O)—CH(NH**)(C₁₋₆ alkyl-NHC(O)—C₁₋₆ alkyl-C(O)***);
L² is **C(O)—;
   **C(O)—C₂₋₄ alkyl-NHC(O)—,
   **C(O)—C₂₋₄ alkyl-[O—C₂₋₆ alkyl]₂₋₄-NHC(O)—,
   **C(O)—C₁₋₄ alkyl-[N(CH₃)—C₂₋₆ alkyl]₂₋₄-NHC(O)—, or
   **C(O)—C₁₋₄ alkyl-[N(CH₃)—C₂₋₆ alkyl]₂₋₄-N(CH₃)C(O)—; and
L³ is ***C(O)—,
   ***C(O)—C₂₋₄ alkyl-NHC(O)—,
   ***C(O)—C₂₋₄ alkyl-[O—C₂₋₆ alkyl]₂₋₄-NHC(O)—,
   ***C(O)—C₁₋₄ alkyl-[N(CH₃)—C₂₋₆ alkyl]₂₋₄-NHC(O)—, or
   ***C(O)—C₁₋₄ alkyl-[N(CH₃)—C₂₋₆ alkyl]₂₋₄-N(CH₃)C(O)—.

In some embodiments, E¹ is —CH₂C(O)OH. In some embodiments, L¹ is —C(O)—C₂₋₄ alkyl-[O—C₂₋₄ alkyl]₂₋₄-NH*.

In some embodiments, A¹ is an amino acid or a derivative thereof. In some embodiments, A¹ is an amino acid derivative comprising an amino acid, wherein the carboxylate group(s) is/are substituted with C₁₋₆ aminoalkyl groups. In some embodiments, A¹ is an amino acid derivative comprising an amino acid (e.g., Glu or Asp), wherein the carboxylate groups are substituted with C₁₋₆ aminoalkyl groups (e.g., beta-alanine groups). In some embodiments, A¹ is an amino acid. In some embodiments, A¹ is lysine. In some embodiments, A¹ is L-Lys. In some embodiments, A¹ is D-Lys. In some embodiments, A¹ is Glu, or an aminoalkyl derivative thereof. In some embodiments, A¹ is L-Glu or a beta-alanine-substituted derivative thereof. In some embodiments, A¹ is D-Glu or a beta-alanine-substituted derivative thereof.

In some embodiments, A¹ is *C(O)—CH(NH**)(C₁₋₄ alkyl-NH***). In some embodiments, A¹ is

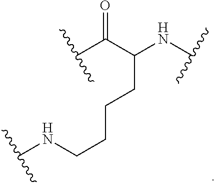 [SMILES: *NCCCCC(N*)C(*)=O]

In some embodiments, A¹ is *C(O)—C₁₋₄ alkyl-C(O)NH—CH(C₁₋₄ alkyl-C(O)NH—C₁₋₄ alkyl-NH**)(C(O)NH—C₁₋₄ alkyl-NH***). In some embodiments, A¹ is

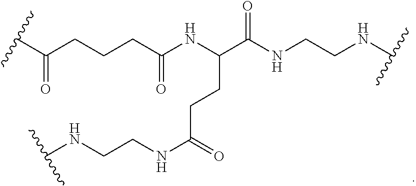 [SMILES: *NCCNC(=O)CCC(NC(=O)CCCC(*)=O)C(=O)NCCN*]

In some embodiments, L² is **C(O)—. In some embodiments, L² is **C(O)—C₂₋₄ alkyl-NHC(O)—. In some embodiments, L² is **C(O)—C₂₋₄ alkyl-[O—C₂₋₆ alkyl]₂₋₄-NHC(O)—.

In some embodiments, L³ is ***C(O)—. In some embodiments, L³ is ***C(O)—C₂₋₄ alkyl-NHC(O)—. In some embodiments, L³ is ***C(O)—C₂₋₄alkyl-[O—C₂₋₆alkyl]₂₋₄-NHC(O)—.

In some embodiments,
E¹ is —CH₂C(O)OH;
L¹ is —C(O)—C₂₋₄ alkyl-[O—C₂₋₄ alkyl]₂₋₄-NH*;
A¹ is *C(O)—CH(NH**)(C₂₋₄ alkyl-NH***);
L² is **C(O)—; and
L³ is ***C(O)—.

In some embodiments,
E¹ is —CH₂C(O)OH;
L¹ is —C(O)—CH₂CH₂—[OCH₂CH₂]₂₋₄—NH*;
A¹ is *C(O)—CH(NH**)((CH₂)₄—NH***);
L² is **C(O)—; and
L³ is ***C(O)—.

In some embodiments,
E¹ is —CH₂C(O)OH;
L¹ is —C(O)—CH₂CH₂—[OCH₂CH₂]₃—NH*;
A¹ is

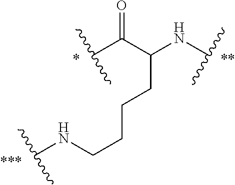 [SMILES: *NCCCCC(N*)C(*)=O]

L² is **C(O)—; and
L³ is ***C(O)—
   wherein:
   * is a bond between L¹ and A¹;
   ** is a bond between A¹ and L²;
   *** is a bond between A¹ and L³.

In some embodiments,
E¹ is —CH₂C(O)OH;
L¹ is —C(O)—C₂₋₄ alkyl-[O—C₂₋₄ alkyl]₂₋₄-NH*;
A¹ is *C(O)—CH(NH**)(C₂₋₄ alkyl-NH***);
L² is **C(O)—C₂₋₄ alkyl-NHC(O)—; and
L³ is ***C(O)—C₂₋₄ alkyl-NHC(O)—.

In some embodiments,
E¹ is —CH₂C(O)OH;
L¹ is —C(O)—CH₂CH₂—[OCH₂CH₂]₂₋₄—NH*;
A¹ is *C(O)—CH(NH**)((CH₂)₄—NH***);
L² is **C(O)—CH₂CH₂—NHC(O)—; and
L³ is ***C(O)—CH₂CH₂—NHC(O)—.

In some embodiments,
E¹ is —CH₂C(O)OH;
L¹ is —C(O)—CH₂CH₂—[OCH₂CH₂]₃—NH*;
A¹

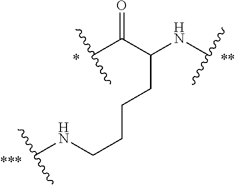 [SMILES: *NCCCCC(N*)C(*)=O]

L² is **C(O)—CH₂CH₂—NHC(O)—; and
L³ is ***C(O)—CH₂CH₂—NHC(O)—;
wherein:
   * is a bond between L¹ and A¹;
   ** is a bond between A¹ and L²;
   *** is a bond between A¹ and L³.

In some embodiments,
E¹ is —CH₂C(O)OH;
L¹ is —C(O)—C₂₋₄ alkyl-[O—C₂₋₄ alkyl]₂₋₄-NH*;
A¹ is *C(O)—CH(NH**)(C₁₋₄ alkyl-NH***);
L² is **C(O)—C₂₋₄ alkyl-[O—C₂₋₄ alkyl]₂₋₄-NHC(O); and
L³ is ***C(O)—C₂₋₄ alkyl-[O—C₂₋₄ alkyl]₂₋₄-NHC(O).

In some embodiments,
E¹ is —CH₂C(O)OH;
L¹ is —C(O)—C₂₋₄ alkyl-[OCH₂CH₂]₂₋₄—NH*;
A¹ is *C(O)—CH(NH**)(C₄ alkyl-NH***);
L² is **C(O)—C₂₋₄alkyl-[OCH₂CH₂]₂₋₄—NHC(O); and
L³ is ***C(O)—C₂₋₄ alkyl-[OCH₂CH₂]₂₋₄—NHC(O).

In some embodiments,
E¹ is —CH₂C(O)OH;
L¹ is —C(O)—CH₂CH₂—[OCH₂CH₂]₃—NH*;
A¹ is

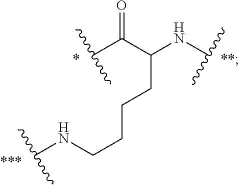 [SMILES: *NCCCCC(N*)C(*)=O]

L² is **C(O)—CH₂CH₂—[OCH₂CH₂]₃—NHC(O); and
L³ is ***C(O)—CH₂CH₂—[OCH₂CH₂]₃—NHC(O).

In some embodiments, A¹ is

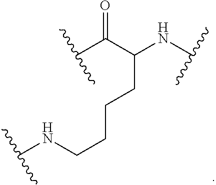 [SMILES: *NCCCCC(N*)C(*)=O]

In some embodiments, A¹ is

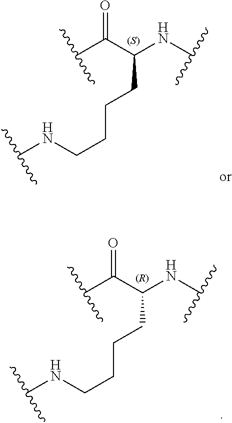

In some embodiments, A¹ is

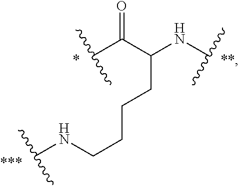 [SMILES: *NCCCCC(N*)C(*)=O]

wherein * is a bond to L¹; ** is a bond to L²; and *** is a bond to L³, or a spanner (S¹, S², S³).

In some embodiments, A¹ is

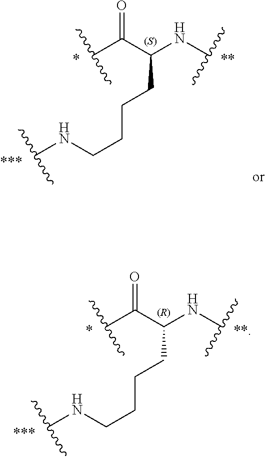

In some embodiments,
E¹ is —CH₂C(O)OH;
L¹ is —C(O)—C₂₋₄ alkyl-[O—C₂₋₄ alkyl]₂₋₄-NH*;
A¹ is *C(O)—CH(NH**)(C₂₋₄ alkyl-NH***);
L² is **C(O)—; and
L³ is ***C(O)—.

In some embodiments, E¹ is —CH₂C(O)NH₂. In some embodiments, L¹ is —C(O)—C₁₋₄ alkyl-[N(CH₃)—C₂₋₄ alkyl]₂₋₄-NH—*. In some embodiments, L¹ is —C(O)—C₁₋₄ alkyl-[N(CH₃)—C₂₋₄ alkyl]₂₋₄-N(CH₃)—*.

In some embodiments,
E¹ is hydrogen or —CH₂C(O)NH₂;
L¹ is —C(O)—C₁₋₄ alkyl-[N(CH₃)—C₂₋₄ alkyl]₂₋₄-NH—*;
A¹ is *C(O)—CH(NH**)(C₂₋₄ alkyl-NH***);
L² is **C(O)—C₂₋₄ alkyl-[O—C₂₋₄ alkyl]₂₋₄-NHC(O); and
L³ is ***C(O)—C₂₋₄ alkyl-[O—C₂₋₄ alkyl]₂₋₄-NHC(O).

In some embodiments,
E¹ is —CH₂C(O)NH₂;
L¹ is —C(O)—C₁₋₄ alkyl-[N(CH₃)CH₂CH₂]₂₋₄—NH—*;
A¹ is *C(O)—CH(NH**)(C₄ alkyl-NH***);
L² is **C(O)—C₂₋₄ alkyl-[OCH₂CH₂]₂₋₄—NHC(O); and
L³ is ***C(O)—C₂₋₄ alkyl-[OCH₂CH₂]₂₋₄—NHC(O).

In some embodiments,
E¹ is hydrogen;
L¹ is —C(O)—C₁₋₄ alkyl-[N(CH₃)CH₂CH₂]₂₋₄—NH—*;
A¹ is *C(O)—CH(NH**)(C₄ alkyl-NH***);
L² is **C(O)—C₂₋₄ alkyl-[OCH₂CH₂]₂₋₄—NHC(O); and
L³ is ***C(O)—C₂₋₄ alkyl-[OCH₂CH₂]₂₋₄—NHC(O).

In some embodiments,
E¹ is hydrogen or —CH₂C(O)NH₂;
L¹ is —C(O)—CH₂—[N(CH₃)CH₂CH₂]₃—NH*;
A¹ is

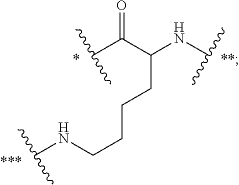 [SMILES: *NCCCCC(N*)C(*)=O]

L² is **C(O)—CH₂CH₂—[OCH₂CH₂]₃—NHC(O); and
L³ is ***C(O)—CH₂CH₂—[OCH₂CH₂]₃—NHC(O).

In some embodiments,
E¹ is hydrogen or —CH₂C(O)NH₂;
L¹ is —C(O)—C₁₋₄ alkyl-[N(CH₃)—C₂₋₄ alkyl]₂₋₄-NH—*;
A¹ is *C(O)—CH(NH**)(C₁₋₄ alkyl-NH***);
L² is **C(O)—C₁₋₄ alkyl-[N(CH₃)—C₂₋₄ alkyl]₂₋₄-NHC(O); and
L³ is ***C(O)—C₁₋₄ alkyl-[N(CH₃)—C₂₋₄ alkyl]₂₋₄-NHC(O).

In some embodiments,
E¹ is —CH₂C(O)NH₂;
L¹ is —C(O)—C₁₋₄ alkyl-[N(CH₃)CH₂CH₂]₂₋₄—NH—*;
A¹ is *C(O)—CH(NH**)(C₄ alkyl-NH***);
L² is **C(O)—C₁₋₄ alkyl-[N(CH₃)CH₂CH₂]₂₋₄—NHC(O); and
L³ is ***C(O)—C₁₋₄ alkyl-[N(CH₃)CH₂CH₂]₂₋₄—NHC(O).

In some embodiments,
E¹ is hydrogen;
L¹ is —C(O)—C₁₋₄ alkyl-[N(CH₃)CH₂CH₂]₂₋₄—NH—*;
A¹ is *C(O)—CH(NH**)(C₄ alkyl-NH***);
L² is **C(O)—C₁₋₄ alkyl-[N(CH₃)CH₂CH₂]₂₋₄—NHC(O); and
L³ is ***C(O)—C₁₋₄ alkyl-[N(CH₃)CH₂CH₂]₂₋₄—NHC(O).

In some embodiments,
E¹ is —CH₂C(O)NH₂;
L¹ is —C(O)—CH₂—[N(CH₃)CH₂CH₂]₃—NH*;
A¹ is

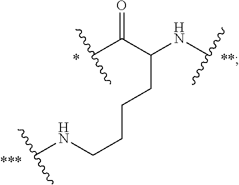 [SMILES: *NCCCCC(N*)C(*)=O]

L² is **C(O)—CH₂—[N(CH₃)CH₂CH₂]₃—NHC(O); and
L³ is ***C(O)—CH₂—[N(CH₃)CH₂CH₂]₃—NHC(O).

In some embodiments, A¹ has a structure represented by the formula:

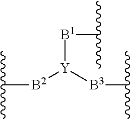

wherein Y is CH, and each of B¹, B², and B³ is heteroalkyl.

In some embodiments, A¹ is:
*C(O)—C₀₋₆ alkyl-Y(C₀₋₆ alkyl-NH**)(C₀₋₆ alkyl-NH***);
*C(O)—C₀₋₆ alkyl-Y(C₀₋₆ alkyl-N(CH₃)**)(C₀₋₆ alkyl-N(CH₃)***);
*C(O)—C₀₋₆ alkyl-C(O)NH—Y(C₀₋₆ alkyl-NH**)(C₀₋₆ alkyl-NH**),
*C(O)—C₀₋₆ alkyl-C(O)NH—Y(C₀₋₆ alkyl-N(CH₃)**)(C₀₋₆ alkyl-N(CH₃)***),
*C(O)—C₀₋₆ alkyl-C(O)N(CH₃)—Y(C₀₋₆ alkyl-NH**)(C₀₋₆ alkyl-NH**),
*C(O)—C₀₋₆ alkyl-C(O)N(CH₃)—Y(C₀₋₆ alkyl-N(CH₃)**)(C₀₋₆ alkyl-N(CH₃)***),
*C(O)—C₀₋₆ alkyl-C(O)NH—Y(C₀₋₆ alkyl-C(O)NH—C₀₋₆ alkyl-NH**)(C₀₋₆ alkyl-C(O)NH—C₀₋₆ alkyl-NH***),
*C(O)—C₀₋₆ alkyl-C(O)NH—Y(C₀₋₆ alkyl-C(O)NH—C₀₋₆ alkyl-N(CH₃)**)(C₀₋₆ alkyl-C(O)NH—C₀₋₆ alkyl-N(CH₃)***),
*C(O)—C₀₋₆ alkyl-C(O)NH—Y(C₀₋₆ alkyl-C(O)N(CH₃)—C₀₋₆ alkyl-N(CH₃)**)(C₀₋₆ alkyl-C(O)N(CH₃)—C₀₋₆ alkyl-N(CH₃)***), or
*C(O)—C₀₋₆ alkyl-C(O)N(CH₃)—Y(C₀₋₆ alkyl-C(O)N(CH₃)—C₀₋₆ alkyl-N(CH₃)**)(C₀₋₆ alkyl-C(O)N(CH₃)—C₀₋₆ alkyl-N(CH₃)***);
   wherein:
   * is a bond to L¹;
   ** is a bond to L²; and
   ** is a bond to L³

In some embodiments, A¹ has a structure represented by the formula:

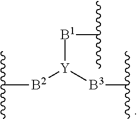

In some embodiments, Y is CH. In some embodiments, B¹ is heteroalkyl. In some embodiments, B¹ is —C(O)—C₀₋₆ alkyl-C(O)NH—. In some embodiments, B¹ is —C(O)—C₂₋₄ alkyl-C(O)NH—. In some embodiments, B¹ is —C(O)—C₃ alkyl-C(O)NH—. In some embodiments, B¹ is —C(O)CH₂CH₂CH₂C(O)NH—. In some embodiments, B² is heteroalkyl. In some embodiments, B² is —C₀₋₆ alkyl-C(O)NH—C₀₋₆ alkyl-NH—. In some embodiments, B² is —C₁₋₄ alkyl-C(O)NH—C₁₋₄ alkyl-NH—. In some embodiments, B² is —C₂ alkyl-C(O)NH—C₂ alkyl-NH—. In some embodiments, B² is —CH₂CH₂C(O)NHCH₂CH₂NH—. In some embodiments, B³ is heteroalkyl. In some embodiments, B³ is —C(O)NH—C₀₋₆ alkyl-NH—. In some embodiments, B3 is —C(O)NH—C₁₋₄ alkyl-NH—. In some embodiments, B3 is —C(O)NH—C₂ alkyl-NH—. In some embodiments, B3 is —C(O)NHCH₂CH₂NH—.

In some embodiments, A¹ has a structure represented by the formula:

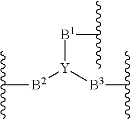

wherein:
Y is CH;
B¹ is *C(O)—(C₁₋₅ alkyl)-C(O)NH—;
B² is —(C₁₋₂ alkyl)-C(O)NH—(C₂₋₆ alkyl)-NH**;
B³ is —C(O)NH—(C₂₋₆ alkyl)-NH***;
wherein:
   * is a bond to L¹;
   ** is a bond to L²; and
   *** is a bond to L³

In some embodiments,
B¹ is *C(O)CH₂CH₂CH₂C(O)NH—;
B² is —CH₂CH₂C(O)NHCH₂CH₂NH**;
B³ is —C(O)NHCH₂CH₂NH***.

In some embodiments, A¹ is *C(O)—C₁₋₄ alkyl-C(O)NH—CH(C₁₋₄ alkyl-C(O)NH—C₁₋₄ alkyl-NH**)(C(O)NH—C₁₋₄ alkyl-NH***).

In some embodiments, A¹ is *C(O)CH₂CH₂CH₂C(O)NH—CH(CH₂CH₂C(O)NHCH₂CH₂NH**)(C(O)NHCH₂CH₂NH**).

In some embodiments, A¹ is

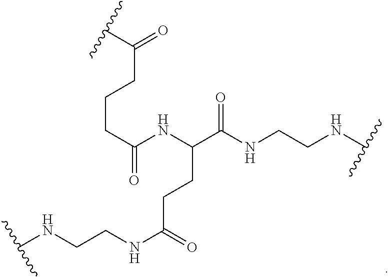 [SMILES: *NCCNC(=O)CCC(NC(=O)CCCC(*)=O)C(=O)NCCN*]

In some embodiments, A¹ is

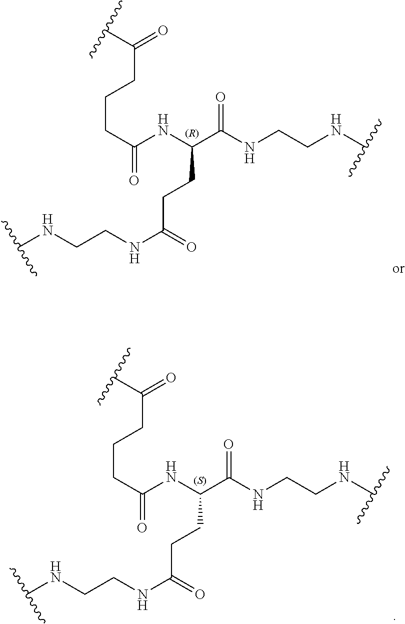

In some embodiments, A¹ is

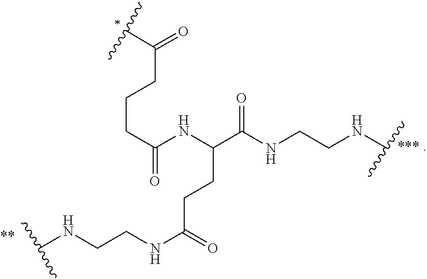 [SMILES: *NCCNC(=O)CCC(NC(=O)CCCC(*)=O)C(=O)NCCN*]

In some embodiments, A¹ is H or H

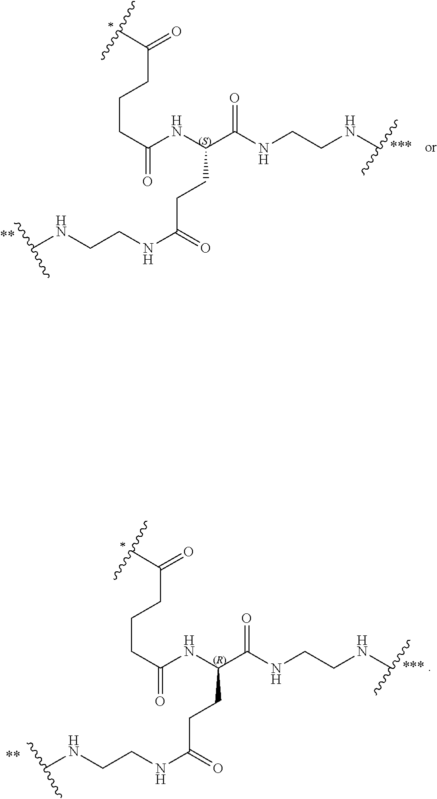

In some embodiments,
E¹ is hydrogen or —CH₂C(O)NH₂;
L¹ is —C(O)—C₁₋₄ alkyl-[N(CH₃)—C₂₋₄ alkyl]₂₋₄-NH—*;
A¹ is *C(O)—C₁₋₄ alkyl-C(O)NH—CH(C₁₋₄ alkyl-C(O)NH—C₁₋₄ alkyl-NH**)(C(O)NH—C₁₋₄ alkyl-NH***;
L² is **C(O)—C₂₋₄ alkyl-[O—C₂₋₄ alkyl]₂₋₄-NHC(O); and
L³ is ***C(O)—C₂₋₄ alkyl-[O—C₂₋₄ alkyl]₂₋₄-NHC(O).

In some embodiments,
E¹ is —CH₂C(O)NH₂;
L¹ is —C(O)—C₁₋₄ alkyl-[N(CH₃)CH₂CH₂]₂₋₄—NH—*;
A¹ is *C(O)CH₂CH₂CH₂C(O)NH—CH—(CH₂CH₂C(O)NHCH₂CH₂NH**)(C(O)NHCH₂CH₂NH***) L² is **C(O)—C₂₋₄alkyl-[OCH₂CH₂]₂₋₄—NHC(O); and
L³ is ***C(O)—C₂₋₄ alkyl-[OCH₂CH₂]₂₋₄—NHC(O).

In some embodiments,
E¹ is hydrogen;
L¹ is —C(O)—C₁₋₄ alkyl-[N(CH₃)CH₂CH₂]₂₋₄—NH—*;
A¹ is *C(O)CH₂CH₂CH₂C(O)NH—CH—(CH₂CH₂C(O)NHCH₂CH₂NH**)(C(O)NHCH₂CH₂NH**) L² is **C(O)—C₂₋₄ alkyl-[OCH₂CH₂]₂₋₄—NHC(O); and
L³ is ***C(O)—C₂₋₄ alkyl-[OCH₂CH₂]₂₋₄—NHC(O).

In some embodiments,
E¹ is —CH₂C(O)NH₂;
L¹ is —C(O)CH₂—[N(CH₃)CH₂CH₂]₃—NH*;
A¹ is

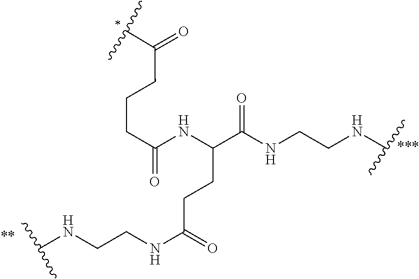 [SMILES: *NCCNC(=O)CCC(NC(=O)CCCC(*)=O)C(=O)NCCN*]

L² is **C(O)CH₂CH₂—[OCH₂CH₂]₃—NHC(O); and
L³ is ***C(O)CH₂CH₂—[OCH₂CH₂]₃—NHC(O).

In some embodiments, provided herein is a compound of Formula (III-B):

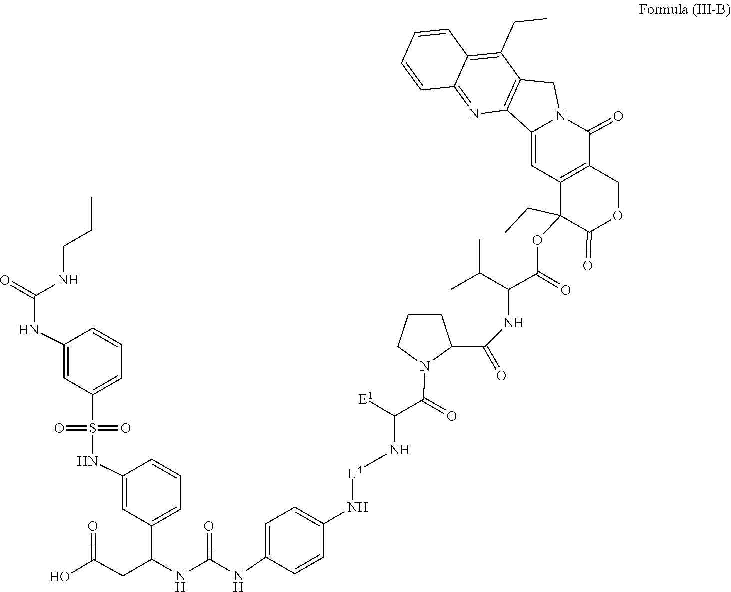

wherein:
E¹ is hydrogen, —CH₂C(O)NH₂, —CH₂C(O)OH, —CH₂C(O)OR¹;
R¹ is C₁₋₁₂ alkyl substituted with —NHL⁶-IN, —N(C₁₋₆alkyl)₂, or —N(C₁₋₆alkyl)₃⁺;
L⁴ is a group of the formula:

—(CO)ᵣ—(CH₂)ₛ—(NR¹⁰—C₁₋₆ alkyl)ₜ-(NR¹¹)ᵤ—(CO)ᵥ—; or

—(CO)ᵣ—(CH₂)ₛ—(NR¹⁰C(O)—C₁₋₆ alkyl)ₜ-(NR¹¹)ᵤ—(CO)ᵥ—;
wherein:
R¹⁰ and R¹¹ are, in each instance, independently hydrogen or C₁₋₃ alkyl;
r is 0 or 1; s is 0-10; t is 1-10; u is 0 or 1; and v is 0 or 1. In some embodiments, provided herein is a compound of Formula (III-B), wherein:
E¹ is hydrogen, —CH₂C(O)NH₂, —CH₂C(O)OH, —CH₂C(O)OR¹;
R¹ is C₁₋₁₂ alkyl substituted with —NHL⁶-IN, —N(C₁₋₆alkyl)₂, or —N(C₁₋₆alkyl)₃⁺;
L⁴ is —C(O)—C₁₋₆ alkyl-[NH—C₁₋₆ alkyl]₁₋₈-NHC(O)—,
   —C(O)—C₁₋₆ alkyl-[N(CH₃)—C₁₋₆ alkyl]₁₋₈-NHC(O)—,
   —C(O)—C₁₋₆ alkyl-[N(CH₃)—C₁₋₆ alkyl]₁₋₈-N(CH₃)—C(O)—,
   —C(O)—C₁₋₆ alkyl-[N(CH₃)C(O)—C₁₋₆ alkyl]₁₋₈-NHC(O)—, or
   —C(O)—C₁₋₆ alkyl-[N(CH₃)C(O)—C₁₋₆ alkyl]₁₋₈-N(CH₃)—C(O)—; and
L⁶ is —C(O)—;
or a pharmaceutically acceptable salt thereof, or a stereoisomer or mixture of stereoisomers thereof.

In some embodiments, provided herein is a compound of Formula (III-B), or a pharmaceutically acceptable salt thereof, or a stereoisomer or mixture of stereoisomers thereof, wherein:
E¹ is hydrogen, —CH₂C(O)NH₂, or —CH₂C(O)OH; and
L⁴ is —C(O)—C₁₋₄ alkyl-[N(CH₃)—C₂₋₄ alkyl]₂₋₄-NHC(O)—,
   —C(O)—C₁₋₄ alkyl-[N(CH₃)—C₂₋₄ alkyl]₂₋₄-N(CH₃)—C(O)—,
   —C(O)—C₁₋₆ alkyl-[N(CH₃)C(O)—C₁₋₆ alkyl]₁₋₈-NHC(O)—, or
   —C(O)—C₁₋₆ alkyl-[N(CH₃)C(O)—C₁₋₆ alkyl]₁₋₈-N(CH₃)—C(O)—.

In some embodiments, E¹ is —CH₂C(O)NH₂. In some embodiments, E¹ is —CH₂C(O)NH₂; and L⁴ is —C(O)—C₁₋₄ alkyl-[N(CH₃)—C₂₋₄ alkyl]₂₋₄-N(CH₃)C(O)— or —C(O)—C₁₋₄ alkyl-[N(CH₃)C(O)—C₁₋₄ alkyl]₂₋₄-N(CH₃)C(O)—. In some embodiments, E¹ is —CH₂C(O)NH₂; and L⁴ is —C(O)—C₁₋₆ alkyl-[N(CH₃)CH₂CH₂]₂—N(CH₃)C(O)—. In some embodiments, E¹ is —CH₂C(O)NH₂; and L⁴ is —C(O)—CH₂—[N(CH₃)CH₂CH₂]₂—N(CH₃)C(O)—.

In some embodiments, E¹ is —CH₂C(O)NH₂, and L⁴ is —C(O)—C₁₋₆ alkyl-[N(CH₃)—C₂₋₆ alkyl]₁₋₈-NHC(O)—. In some embodiments E¹ is —CH₂C(O)NH₂; and L⁴ is —C(O)—C₁₋₆ alkyl-[N(CH₃)CH₂CH₂]₃—NHC(O)—. In some embodiments, E¹ is —CH₂C(O)NH₂; and L⁴ is —C(O)—CH₂—[N(CH₃)CH₂CH₂]₃—NHC(O)—. In some embodiments, E¹ is —CH₂C(O)NH₂; and L⁴ is —C(O)—CH₂—[N(CH₃)C(O)CH₂]₃—N(CH₃)C(O)—.

In some embodiments, E¹ is hydrogen. In some embodiments, E¹ is hydrogen; and L⁴ is —C(O)—C₁₋₆ alkyl-[N(CH₃)—C₂₋₆ alkyl]₁₋₈-NHC(O)—. In some embodiments, E¹ is hydrogen; and L⁴ is —C(O)—C₁₋₆ alkyl-[N(CH₃)CH₂CH₂]₂—NHC(O)—. In some embodiments, E^{t} is hydrogen; and L⁴ is —C(O)—CH₂—[N(CH₃)CH₂CH₂]₂—NHC(O)—.

In some embodiments, E¹ is —CH₂C(O)OH. In some embodiments, E¹ is —CH₂C(O)OH; and L⁴ is —C(O)—C₁₋₄ alkyl-[N(CH₃)—C₂₋₄ alkyl]₂₋₄-NHC(O)—. In some embodiments, E¹ is —CH₂C(O)OH; and L⁴ is —C(O)—C₁₋₆ alkyl-[N(CH₃)C(O)—C₁₋₆ alkyl]₁₋₈-N(CH₃)—C(O)—. In some embodiments, E¹ is —C(O)—CH₂—[N(CH₃)C(O)CH₂]₁₋₈—N(CH₃)C(O)—.

In some embodiments, provided herein is a compound of Formula (III-C1):

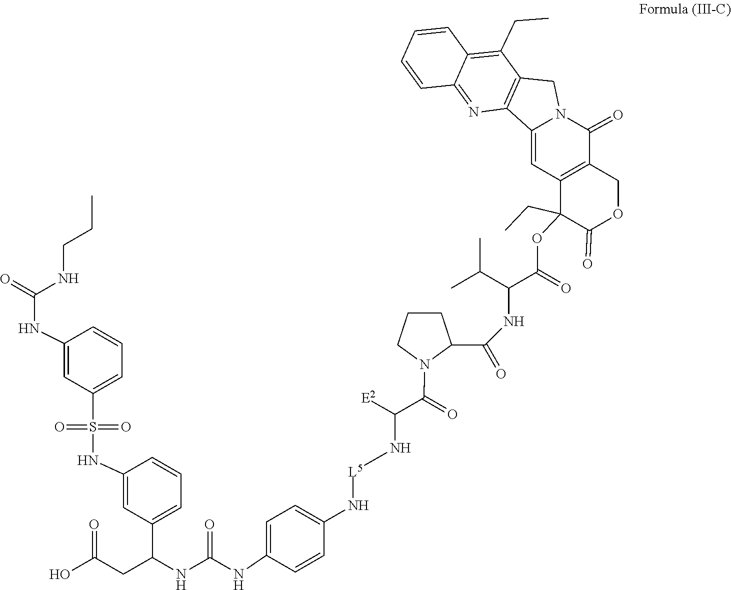

wherein:
E² is —CH₂C(O)OR¹ or —CH₂CH₂C(O)OR¹;
R¹ is C₁₋₁₂ alkyl substituted with —NHC(O)—IN, —N(C₁₋₆alkyl)₂, or —N(C₁₋₆alkyl)₃m; and
L⁵ is a bivalent linker (e.g., a substituted or unsubstituted alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker). In some embodiments, L⁵ is a bivalent linker selected from the group consisting of simple spacers, polymeric spacers, and compound spacers, each defined herein. In some embodiments, L⁵ is a polymeric spacer.

In some embodiments, provided herein is a compound of Formula (III-C1), wherein:
E² is —CH₂C(O)OR¹ or —CH₂CH₂C(O)OR¹;
R¹ is C₁₋₁₂ alkyl substituted with —NHL⁶-IN, —N(C₁₋₆alkyl)₂, or —N(C₁₋₆alkyl)₃⁺;
L⁵ is —C(O)—C₁₋₆ alkyl-[O—C₂₋₆ alkyl]₁₋₈-NHC(O)—,
   —C(O)—C₁₋₆ alkyl-[NH—C₂₋₆ alkyl]₁₋₈-NHC(O)—,
   —C(O)—C₁₋₆ alkyl-[N(CH₃)—C₂₋₆ alkyl]₁₋₈-NHC(O)—,
   —C(O)—C₁₋₆ alkyl-[N(CH₃)—C₂₋₆ alkyl]₁₋₈-N(CH₃)—C(O)—;
   —C(O)—C₁₋₆ alkyl-[N(CH₃)C(O)—C₁₋₆ alkyl]₁₋₈-NHC(O)—, or
   —C(O)—C₁₋₆ alkyl-[N(CH₃)C(O)—C₁₋₆ alkyl]₁₋₈-N(CH₃)C(O)—; and
L⁶ is —C(O)—;
   or a pharmaceutically acceptable salt thereof, or a stereoisomer or mixture of stereoisomers thereof.

In some embodiments, L⁵ is —C(O)—C₂₋₆ alkyl-[O—C₂₋₆ alkyl]₁₋₈-NHC(O)—. In some embodiments, L⁵ is —C(O)—C₁₋₆ alkyl-[N(CH₃)—C₂₋₆ alkyl]₁₋₈-NHC(O)—. In some embodiments, L⁵ is —C(O)—C₁₋₆ alkyl-[N(CH₃)—C₁₋₆ alkyl]₁₋₈-N(CH₃)C(O)—. In some embodiments, provided herein is a compound of Formula (III-C), or a pharmaceutically acceptable salt thereof, or a stereoisomer or mixture of stereoisomers thereof, wherein:
E² is —CH₂C(O)OR¹ or —CH₂CH₂C(O)OR¹;
R¹ is C₂₋₆ alkyl-N(CH₃)₂, C₂₋₆ alkyl-N(CH₃)₃⁺, or C₂₋₆ alkyl-NHC(O)-IN; and
L⁵ is —C(O)—C₂₋₄ alkyl-[O—C₂₋₄ alkyl]₂₋₄-NHC(O)—,
   —C(O)—C₁₋₄ alkyl-[N(CH₃)—C₂₋₄ alkyl]₂₋₄-NHC(O)—, or
   —C(O)—C₁₋₆ alkyl-[N(CH₃)C(O)—C₁₋₆ alkyl]₁₋₈-N(CH₃)—C(O)—.

In some embodiments, E² is —CH₂C(O)OR¹; R¹ is C₁₋₆ alkyl substituted with —N(C₁₋₆-alkyl)₃⁺. In some embodiments, L⁵ is —C(O)—C₂₋₄ alkyl-[O—C₂₋₄ alkyl]₂₋₄-NHC(O)—. In some embodiments, E² is —CH₂C(O)OR¹; R¹ is C₁₋₆ alkyl substituted with —N(C₁₋₆alkyl)₃⁺; and L⁵ is —C(O)—C₂₋₄ alkyl-[O—C₂₋₄ alkyl]₂₋₄-NHC(O)—. In some embodiments, E² is —CH₂C(O)OR¹; R¹ is C₁₋₆ alkyl substituted with —N(C₁₋₆alkyl)₃⁺; and L⁵ is —C(O)—C₁₋₆ alkyl-[NH—C₂₋₆ alkyl]₁₋₈-NHC(O)—.

In some embodiments, E² is —CH₂C(O)OR¹; R¹ is C₁₋₆ alkyl substituted with —N(C₁₋₆-alkyl)₃⁺; and L⁵ is —C(O)—C₁₋₆ alkyl-[N(CH₃)—C₂₋₆ alkyl]₁₋₈-NHC(O)—. In some embodiments, E² is —CH₂C(O)OR¹; R¹ is C₁₋₆ alkyl substituted with —N(C₁₋₆alkyl)₃⁺; and L⁵ is —C(O)—C₁₋₆ alkyl-[N(CH₃)—C₂₋₆ alkyl]₁₋₈-N(CH₃)—C(O)—.

In some embodiments, E² is —CH₂C(O)O—(CH₂)₁₋₄—N(CH₃)₃⁺. In some embodiments, L⁵ is —C(O)—C₂₋₄ alkyl-[OCH₂CH₂]₂₋₄—NHC(O)—. In some embodiments, E² is —CH₂C(O)O—(CH₂)₁₋₄—N(CH₃)₃⁺; and L⁵ is —C(O)—C₂₋₄ alkyl-[OCH₂CH₂]₂₋₄—NHC(O)—. In some embodiments, E² is —CH₂C(O)OCH₂CH₂N(CH₃)₃⁺. In some embodiments, L⁵ is —C(O)—CH₂CH₂—[OCH₂CH₂]₂—NHC(O)—. In some embodiments, L⁵ is —C(O)—CH₂CH₂—[OCH₂CH₂]₃—NHC(O)—. In some embodiments, L⁵ is —C(O)—CH₂CH₂—[OCH₂CH₂]₄—NHC(O)—.

In some embodiments, provided herein is a compound of Formula (III-C), wherein:
E² is —CH₂C(O)OCH₂CH₂N(CH₃)₃⁺; and
L⁵ is —C(O)—CH₂CH₂—[OCH₂CH₂]₃—NHC(O)—.

In some embodiments, E² is —CH₂C(O)OR¹; R¹ is C₁₋₆ alkyl substituted with —N(C₁₋₆alkyl)₂. In some embodiments, L⁵ is —C(O)—C₂₋₄ alkyl-[O—C₂₋₄ alkyl]₂₋₄-NHC(O)—. In some embodiments, E² is —CH₂C(O)OR¹; R¹ is C₁₋₆ alkyl substituted with —N(C₁₋₆alkyl)₂; and L⁵ is —C(O)—C₂₋₄ alkyl-[O—C₂₋₄ alkyl]₂₋₄-NHC(O)—. In some embodiments, E² is —CH₂C(O)OR¹; R¹ is C₁₋₆ alkyl substituted with —N(C₁₋₆alkyl)₂; and L⁵ is —C(O)—C₁₋₆ alkyl-[NH—C₁₋₆ alkyl]₁₋₈-NHC(O)—.

In some embodiments, E² is —CH₂C(O)OR¹; R¹ is C₁₋₆ alkyl substituted with —N(C₁₋₆alkyl)₂; and L⁵ is —C(O)—C₁₋₆ alkyl-[N(CH₃)—C₁₋₆ alkyl]₁₋₈-NHC(O)—. In some embodiments, E² is —CH₂C(O)OR¹; R¹ is C₁₋₆ alkyl substituted with —N(C₁₋₆alkyl)₂; and L⁵ is —C(O)—C₁₋₆ alkyl-[N(CH₃)—C₁₋₆ alkyl]₁₋₈-N(CH₃)—C(O)—.

In some embodiments, E² is —CH₂C(O)O—(CH₂)₁₋₄—N(C₁₋₆alkyl)₂. In some embodiments, L⁵ is —C(O)—C₂₋₄ alkyl-[OCH₂CH₂]₂₋₄—NHC(O)—. In some embodiments, E² is —CH₂C(O)O—(CH₂)₁₋₄—N(CH₃)₂; and L⁵ is —C(O)—C₂₋₄ alkyl-[OCH₂CH₂]₂₋₄—NHC(O)—. In some embodiments, E² is —CH₂C(O)O(CH₂)₄N(CH₃)₂. In some embodiments, E² is —CH₂C(O)O(CH₂)₃N(CH₃)₂. In some embodiments, E² is —CH₂C(O)O(CH₂)₂N(CH₃)₂. In some embodiments, L⁵ is —C(O)—CH₂CH₂—[OCH₂CH₂]₂—NHC(O)—. In some embodiments, L⁵ is —C(O)—CH₂CH₂—[OCH₂CH₂]₃—NHC(O)—. In some embodiments, L⁵ is —C(O)—CH₂CH₂—[OCH₂CH₂]₄—NHC(O)—.

In some embodiments, provided herein is a compound of Formula (III-C), wherein:
E² is —CH₂C(O)O(CH₂)₄N(CH₃)₂; and
L⁵ is —C(O)—CH₂CH₂—[OCH₂CH₂]₃—NHC(O)—.

In some embodiments, provided herein is a compound of Formula (III-C), wherein:
E² is —CH₂C(O)O(CH₂)₃N(CH₃)₂; and
L⁵ is —C(O)—CH₂CH₂—[OCH₂CH₂]₃—NHC(O)—.

In some embodiments, E² is —CH₂C(O)OR¹; R¹ is C₁₋₆ alkyl substituted with —NHL⁶-IN. In some embodiments, L⁵ is —C(O)—C₂₋₄ alkyl-[O—C₂₋₄ alkyl]₂₋₄-NHC(O)—. In some embodiments, E² is —CH₂C(O)OR¹; R¹ is C₁₋₆ alkyl substituted with —NHL⁶-IN; and L⁵ is —C(O)—C₂₋₄ alkyl-[O—C₂₋₄ alkyl]₂₋₄-NHC(O)—. In some embodiments, E² is —CH₂C(O)OR¹; R¹ is C₁₋₆ alkyl substituted with —NHL⁶-IN; and L⁵ is —C(O)—C₁₋₆ alkyl-[NH—C₂₋₆ alkyl]₁₋₈-NHC(O)—. In some embodiments, E² is —CH₂C(O)OR¹; R¹ is C₁₋₆ alkyl substituted with —NHL⁶-IN; and L⁵ is —C(O)—C₁₋₆ alkyl-[N(CH₃)—C₂₋₆ alkyl]₁₋₈-NHC(O)—. In some embodiments, E² is —CH₂C(O)OR¹; R^{t} is C₁₋₆ alkyl substituted with —NHL⁶-IN; and L⁵ is —C(O)—C₁₋₆ alkyl-[N(CH₃)—C₂₋₆ alkyl]₁₋₈-N(CH₃)—C(O)—.

In some embodiments, E² is —CH₂C(O)O—(CH₂)₁₋₄—NHL⁶-IN. In some embodiments, L⁵ is —C(O)—C₂₋₄ alkyl-[OCH₂CH₂]₂₋₄—NHC(O)—. In some embodiments, E² is —CH₂C(O)O—(CH₂)₁₋₄—NHL⁶-IN; and L⁵ is —C(O)—C₂₋₄ alkyl-[OCH₂CH₂]₂₋₄—NHC(O)—. In some embodiments, E² is —CH₂C(O)O(CH₂)₄NHL⁶-IN. In some embodiments, E² is —CH₂C(O)O(CH₂)₃NHL⁶-IN. In some embodiments, E² is —CH₂C(O)O(CH₂)₂NHL⁶-IN. In some embodiments, L⁶ is —C(O)—. In some embodiments, E² is —CH₂C(O)O(CH₂)₂NHC(O)-IN. In some embodiments, L⁵ is —C(O)—CH₂CH₂—[OCH₂CH₂]₂—NHC(O)—. In some embodiments, L⁵ is —C(O)—CH₂CH₂—[OCH₂CH₂]₃—NHC(O)—. In some embodiments, L⁵ is —C(O)—CH₂CH₂—[OCH₂CH₂]₄—NHC(O)—. In some embodiments, E² is —CH₂C(O)O(CH₂)₄—NHC(O)-IN; and L⁵ is —C(O)—CH₂CH₂—[OCH₂CH₂]₃—NHC(O)—.

In some embodiments, provided herein is a compound of Formula (III-C2):

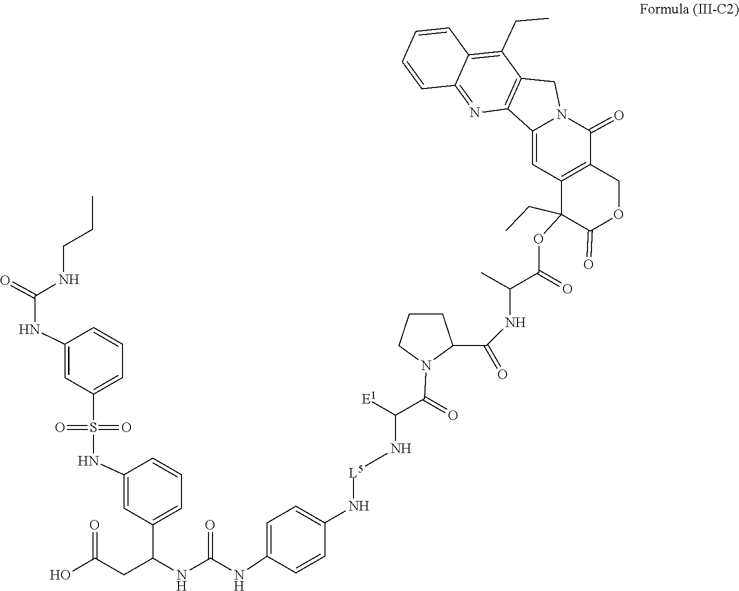

wherein:
E¹ is hydrogen, —CH₃, —CH₂CH₃, —CH₂CH₂CH₃, —CH₂C(O)NH₂, —CH₂C(O)OH, —CH₂C(O)OR¹;
R¹ is C₁₋₁₂ alkyl substituted with —NHC(O)—IN, —N(C₁₋₆alkyl)₂, or —N(C₁₋₆alkyl)₃⁺; and
L⁵ is a bivalent linker (e.g., a substituted or unsubstituted alkyl, substituted or unsubstituted heteroalkyl, substituted or unsubstituted aralkyl, substituted or unsubstituted heteroaralkyl, substituted or unsubstituted heteroalkyl-aryl, or a substituted or unsubstituted heteroalkyl-heteroaryl linker).

In some embodiments, E¹ is —CH₂C(O)NH₂, or —CH₂C(O)OH. In some embodiments, E¹ is —CH₂C(O)NH₂. In some embodiments, E¹ is —CH₂C(O)OH. In some embodiments, E¹ is —CH₂C(O)NH₂, or —CH₂C(O)OH; and L⁵ is a polymeric spacer. In some embodiments, E¹ is —CH₂C(O)NH₂, or —CH₂C(O)OH; and L⁵ is a linker having a structure represented by the formula (i), (ii), or (iii):

—(CO)ₘ—(CH₂)ₙ—(OC₂₋₆ alkyl)ₒ-(NH)ₚ—(CO)_{q}—; (i)

—(CO)ᵣ—(CH₂)ₛ—(NR¹⁰C₂₋₆ alkyl)ₜ-(NR¹¹)ᵤ—(CO)ᵥ—; or (ii)

—(CO)ᵣ—(CH₂)ₛ—(NR¹⁰C(O)C₁₋₆ alkyl)ₜ-(NR¹¹)ᵤ—(CO)ᵥ—; (iii)
wherein:
each R¹⁰ and R¹¹ is independently hydrogen or C₁₋₃ alkyl;
each m, p, q, r, u, and v is independently 0 or 1; and
each n, o, s, and t is independently 0, 1, 2, 3, 4, 5, 6, 7, 8, 9, or 10.

In some embodiments,
E¹ is —CH₂C(O)NH₂, or —CH₂C(O)OH; and
L⁵ is —C(O)—C₁₋₆ alkyl-[O—C₂₋₆ alkyl]₁₋₈-NHC(O)—,
   —C(O)—C₁₋₆ alkyl-[NH—C₁₋₆ alkyl]₁₋₈-NHC(O)—,
   —C(O)—C₁₋₆ alkyl-[N(CH₃)—C₁₋₆ alkyl]₁₋₈-NHC(O)—,
   —C(O)—C₁₋₆ alkyl-[N(CH₃)—C₁₋₆ alkyl]₁₋₈-N(CH₃)—C(O)—;
   —C(O)—C₁₋₆ alkyl-[N(CH₃)C(O)—C₁₋₆ alkyl]₁₋₈-NHC(O)—, or
   —C(O)—C₁₋₆ alkyl-[N(CH₃)C(O)—C₁₋₆ alkyl]₁₋₈-N(CH₃)C(O)—;

In some embodiments,
E¹ is —CH₂C(O)NH₂, or —CH₂C(O)OH; and
L⁵ is —C(O)—CH₂CH₂—[OCH₂CH₂]₂₋₄—NHC(O)—;
   —C(O)—CH₂—[N(CH₃)CH₂CH₂]₂₋₄—NHC(O)—;
   —C(O)—CH₂—[N(CH₃)CH₂CH₂]₂₋₄—N(CH₃)C(O)—; or
   —C(O)—CH₂—[N(CH₃)C(O)CH₂]₂₋₄—N(CH₃)C(O)—.

In some embodiments, provided herein is a compound of Formula (III-D):

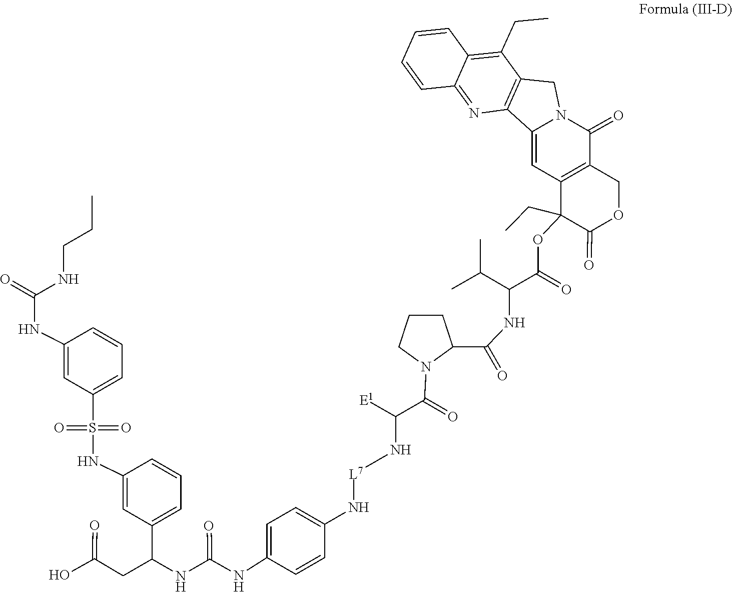

wherein:
E¹ is hydrogen, —CH₃, —CH₂CH₃, —CH₂CH₂CH₃, —CH₂C(O)NH₂, —CH₂C(O)OH, —CH₂C(O)OR¹;
L⁷ is a substituted or unsubstituted C₁₋₆₀ alkyl, or substituted or unsubstituted heteroalkyl comprising 4-12 heteroalkyl units, each independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, or —NHS(O)₂NHC(O)—;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, —C₁₋₆ alkyl, —CN, —CONH₂, —CONH(C₁₋₆ alkyl), —CON(C₁₋₆alkyl)₂, —COOH, —COO(C₁₋₆ alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl;
   or a pharmaceutically acceptable salt thereof, or a stereoisomer or mixture of stereoisomers thereof.

In some embodiments, provided herein is a compound of Formula (III-D), wherein L⁷ is an elongated spacer selected from the list defined herein. In some embodiments, provided herein is a compound of Formula (III-D), wherein L⁷ is an elongated spacer represented by formula (i) or (ii) below:

—(CO)ₐ(C₀₋₆ alkyl)(Z¹C₁₋₆ alkyl)_{b}-Z²—(C₀₋₆ alkyl)(Z³C₁₋₆ alkyl)_{c}(Z⁴)_{d}(CO)ₑ—; or (i)

—(CO)ₐ—(C₀₋₆ alkyl)-(Z¹C(O)C₁₋₆ alkyl)_{b}-Z²—; (ii)
wherein:
Z¹ is in each occurrence independently —O—, —NH—, or —N(C₁₋₃ alkyl)-;
Z² is a bond, —C(O)—, —C(O)NH—, —C(O)—(C₁₋₆ alkyl)-C(O)—, —C(O)—(C₁₋₆ alkyl)-C(O)NH—, —C(O)—(C₁₋₆ alkyl)-C(O)N(CH₃)—, —C(O)NH—(C₁₋₆ alkyl)-C(O)NH—, —NH—, —N(C₁₋₆ alkyl)-, —N(C₁₋₆ alkyl)C(O)—, —NHC(O)—, —NHC(O)NH—, or —NHC(O)—(C₁₋₆ alkyl)-NHC(O)—;
Z³ is in each occurrence independently —O—, —NH—, or —N(C₁₋₃ alkyl)-;
Z⁴ is —O—, —NH—, or —N(C₁₋₃ alkyl)-; and
a is 0 or 1; b is 1 to 10; c is 1 to 10; d is 0 or 1; and e is 0 or 1.

In some embodiments, L⁷ is an elongated spacer (e.g., a substituted or unsubstituted C₁₋₃₀ alkyl, or substituted or unsubstituted heteroalkyl containing 4-12 Z groups; wherein each Z group is independently —(O—C₂₋₆ alkyl)-, —(NH—C₂₋₆ alkyl)-, —(N(C₁₋₃ alkyl)-C₂₋₆ alkyl), or —(N(C₁₋₃ alkyl)C(O)—C₁₋₆ alkyl)). In some embodiments, L⁷ is an elongated spacer (e.g., a substituted or unsubstituted C₁₋₃₀ alkyl, or substituted or unsubstituted heteroalkyl containing 4-12 Z groups; wherein each Z group is independently —(O—C₂₋₆ alkyl)-, —(N(CH₃)—C₁₋₆ alkyl), or —(N(CH₃)C(O)—C₁₋₆ alkyl)).

In some embodiments, provided herein is a compound of Formula (III-D), wherein:
E¹ is hydrogen, —CH₂C(O)NH₂, —CH₂C(O)OH, —CH₂C(O)OR¹;
R^{t} is C₁₋₁₂ alkyl substituted with —NHL⁶-IN, —N(C₁₋₆alkyl)₂, or —N(C₁₋₆alkyl)₃⁺;
L⁶ is —C(O)—; and
L⁷ is —C(O)—C₂₋₄ alkyl-[O—C₂₋₄ alkyl]₂₋₄-NHC(O)—C₂₋₄ alkyl-[O—C₁₋₃ alkyl]₂₋₄-NHC(O)—, —C(O)—C₁₋₄ alkyl-[NH—C₂₋₄ alkyl]₂₋₄-NHC(O)—C₁₋₄ alkyl-[NH—C₁₋₃ alkyl]₂₋₄-NHC(O)—; —C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—, —C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—, or —C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—;
or a pharmaceutically acceptable salt thereof, or a stereoisomer or mixture of stereoisomers thereof.

In some embodiments, E¹ is —CH₂C(O)NH₂, and L⁷ is —C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—.

In some embodiments, E¹ is —CH₂C(O)NH₂; and L⁷ is —C(O)—C₁₋₄ alkyl-[N(CH₃)CH₂CH₂]₂₋₄—NHC(O)—C₁₋₄ alkyl-[N(CH₃)CH₂CH₂]₂₋₄—NHC(O)—.

In some embodiments, provided herein is a compound of Formula (III-D), wherein:
E¹ is —CH₂C(O)NH₂ or —CH₂C(O)OH; and
L⁷ is —C(O)—CH₂—[N(CH₃)CH₂CH₂]₃—NHC(O)—CH₂—[N(CH₃)CH₂CH₂]₃—NHC(O)—; or —C(O)—CH₂—[N(CH₃)C(O)CH₂]₃—N(CH₃)C(O)CH₂—[N(CH₃)C(O)CH₂]₃—N(CH₃)C(O)—.

In some embodiments, provided herein is a compound of Formula (III-D), wherein E¹ is —CH₂C(O)NH₂ or —CH₂C(O)OH; and L⁷ is:

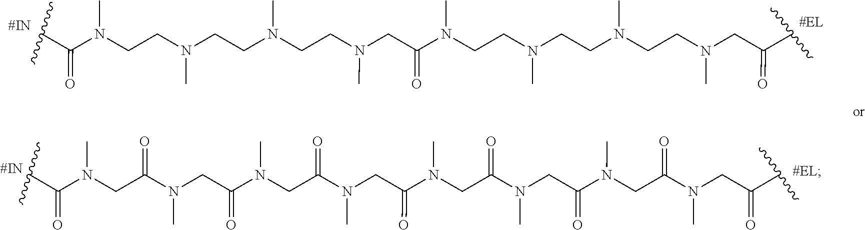 [SMILES: *C(=O)CN(C)CCN(C)CCN(C)CCN(C)C(=O)CN(C)CCN(C)CCN(C)CCN(C)C(*)=O ; *C(=O)CN(C)C(=O)CN(C)C(=O)CN(C)C(=O)CN(C)C(=O)CN(C)C(=O)CN(C)C(=O)CN(C)C(=O)CN(C)C(*)=O]

wherein #IN represents a bond to an integrin binder (IN), and #EL represents a bond to an enzymatically-cleavable linker (EL).

In some embodiments, provided herein is a compound of Formula (III-E):

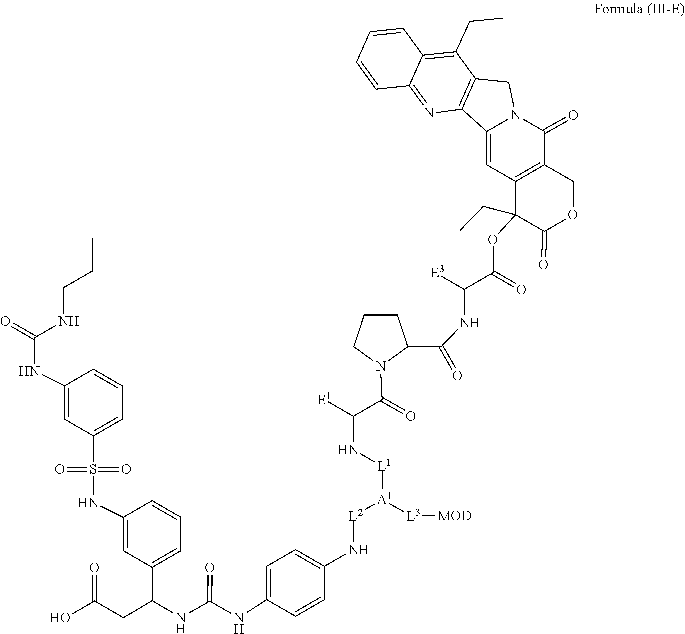

wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R¹ is C₁₋₁₂ alkyl substituted with —NHL⁶-IN, —N(C₁₋₆alkyl)₂, or —N(C₁₋₆alkyl)₃⁺; each L¹, L², L³, and L⁶ is independently a bivalent linker;
A¹ is a trivalent linker; and
MOD is a physicochemical or pharmacokinetic modulator.

In some embodiments, provided herein is a compound of Formula (III-E), wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R¹ is C₁₋₁₂ alkyl substituted with —NHL⁶-IN, —NHL⁶-MOD, —N(C₁₋₆alkyl)₂, or —N(C₁₋₆alkyl)₃;
L¹ is —C(O)—C₁₋₆ alkyl-[O—C₂₋₆ alkyl]₁₋₈-NH*,
   —C(O)—C₁₋₆ alkyl-[N(CH₃)—C₁₋₆ alkyl]₁₋₈-NH—*,
   —C(O)—C₁₋₆ alkyl-[N(CH₃)C(O)—C₁₋₆ alkyl]₁₋₈-NH—*,
   —C(O)—C₁₋₆ alkyl-[N(CH₃)—C₁₋₆ alkyl]₁₋₈-N(CH₃)—*;
   —C(O)—C₁₋₆ alkyl-[N(CH₃)C(O)—C₁₋₆ alkyl]₁₋₈-N(CH₃)—*;
A¹ is a trivalent radical
L² is C(O)—,
   **C(O)—C₁₋₆ alkyl-NHC(O)—,
   **C(O)—C₁₋₆ alkyl-[O—C₂₋₆ alkyl]₁₋₈-NHC(O)—,
   **C(O)—C₁₋₆ alkyl-[N(CH₃)—C₁₋₆ alkyl]₁₋₈-NHC(O)—,
   **C(O)—C₁₋₆ alkyl-[N(CH₃)C(O)—C₁₋₆ alkyl]₁₋₈-NHC(O)—,
   **C(O)—C₁₋₆ alkyl-[N(CH₃)—C₁₋₆ alkyl]₁₋₈-N(CH₃)C(O)—;
   **C(O)—C₁₋₆ alkyl-[N(CH₃)C(O)—C₁₋₆ alkyl]₁₋₈-N(CH₃)C(O)—;
L³ is —C₁₋₁₂ alkyl-,
   ***—C₁₋₁₂ alkyl-NH—,
   ***—NH—C₁₋₁₂ alkyl-,
   ***—NH—C₁₋₁₂ alkyl-NH—,
   ***—C(O)—C₁₋₁₂ alkyl-,
   ***—C(O)—C₁₋₁₂ alkyl-NH—;
   ***C(O)—C₁₋₆ alkyl-[O—C₂₋₆ alkyl]₁₋₈-NH—,
   ***C(O)—C₁₋₆ alkyl-[N(CH₃)—C₁₋₆ alkyl]₁₋₈-NH—,
   ***C(O)—C₁₋₆ alkyl-[N(CH₃)—C₁₋₆ alkyl]₁₋₈-N(CH₃)—;
   ***C(O)—C₁₋₆ alkyl-[N(CH₃)C(O)—C₁₋₆ alkyl]₁₋₈-N(CH₃)—;
L⁶ is —C(O)—, —C₁₋₆ alkyl-, —C₁₋₆ alkyl-NH—, —NH—, or —NHC(O)—; and
MOD is —COOH.

In some embodiments, MOD is —COOH (e.g., —COO⁻Na⁺), —COOCH₃, —NH₂, —N(CH₃)₂, —N(CH₃)₃, —NC(═NH)NH₂, —OH, —O⁻Na⁺, or —OCH₃. In some embodiments, MOD is —OH, —COOH, —NH₂, NHCH₃, —N(CH₃)₂, —N(CH₃)₃⁺, or a salt thereof. In some embodiments, MOD is —COOH. In some embodiments, MOD is —OH. In some embodiments, MOD is —H or —CH₃. In some embodiments, L³ is C₁₋₁₂ alkyl. In some embodiments, L³ is NHC₁₋₁₂ alkyl. In some embodiments, L³ is NHC(O)—C₁₋₁₂ alkyl. In some embodiments, L³ is C(O)—C₁₋₁₂ alkyl. In some embodiments, L³ is a heteroalkyl linker. In some embodiments, L³ is a simple spacer. In some embodiments, L³ is a carbonyl, an acyl linker, an amide linker, or an alkyl linker. In some embodiments, L³ is an optionally substituted alkyl linker (e.g., an optionally substituted alkyl amide linker). In some embodiments, MOD is —OH, —COOH, —NH₂, NHCH₃, —N(CH₃)₂, —(CH₃)₃f, or a salt thereof. In some embodiments, L³ is C₁₋₁₂ alkyl. In some embodiments, L³ is NHC₁₋₁₂ alkyl. In some embodiments, L³ is NHC(O)—C₁₋₁₂ alkyl. In some embodiments, L³ is C(O)—C₁₋₁₂ alkyl.

In some embodiments, A¹ is:

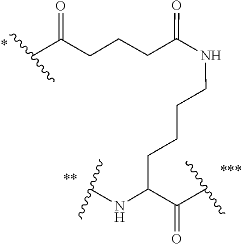 [SMILES: *NC(CCCCNC(=O)CCCC(*)=O)C(*)=O]

wherein: * denotes a bond to L¹, ** denotes a bond to L², *** denotes a bond to L³.

In some embodiments, provided herein is a compound having a structure:

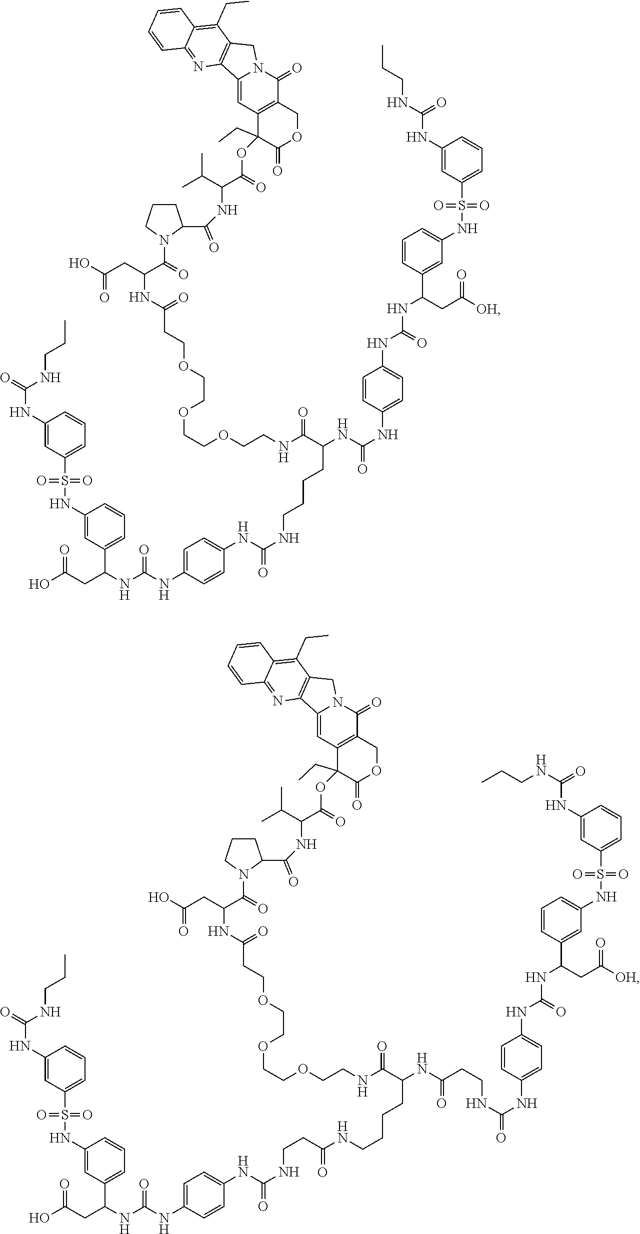

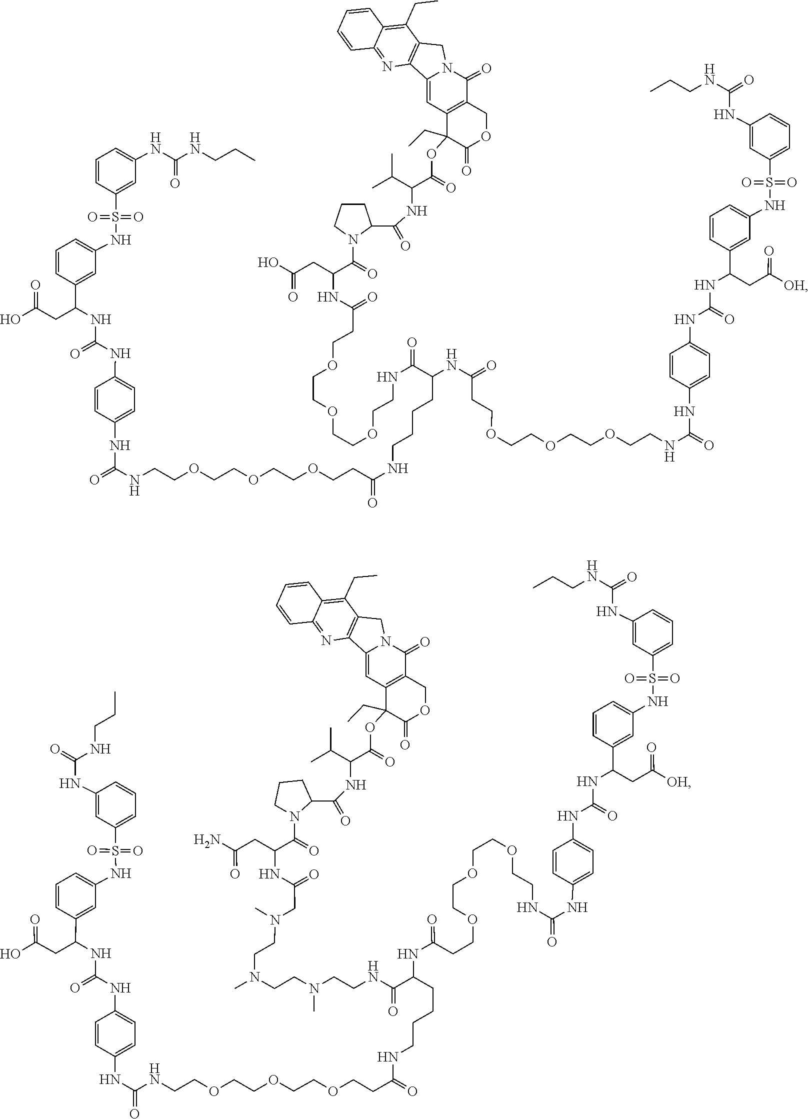

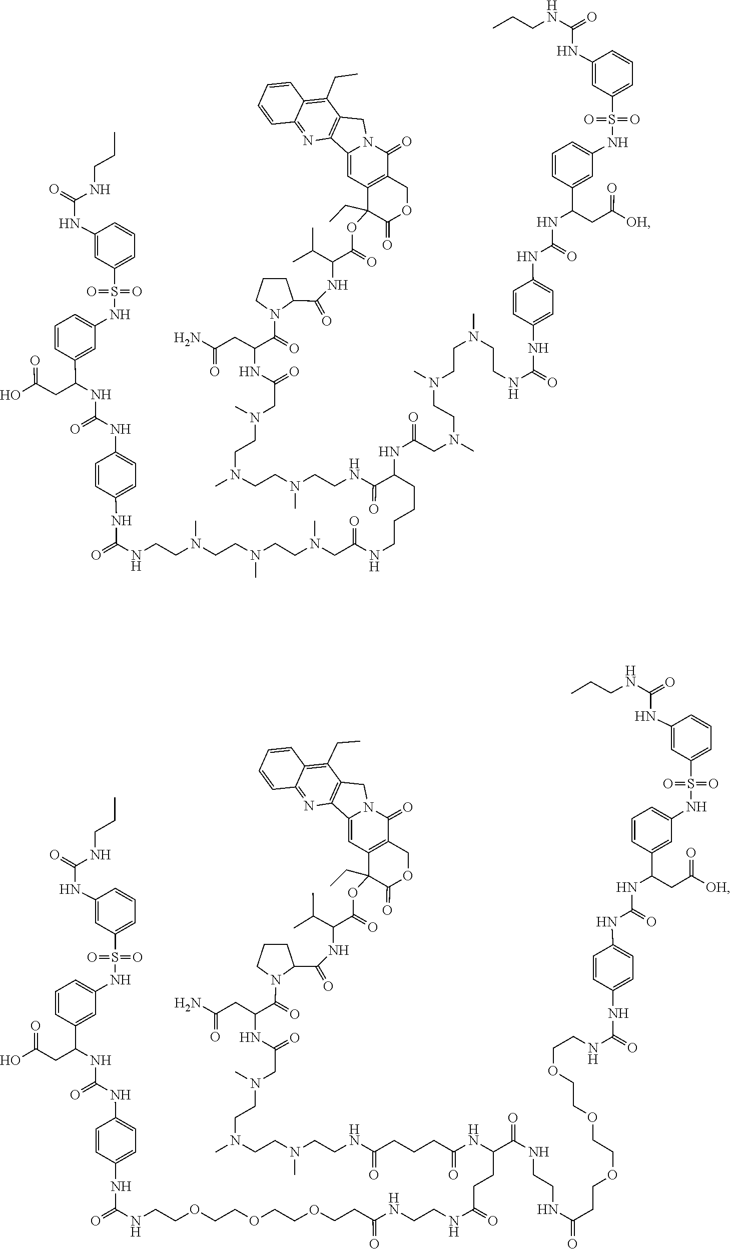

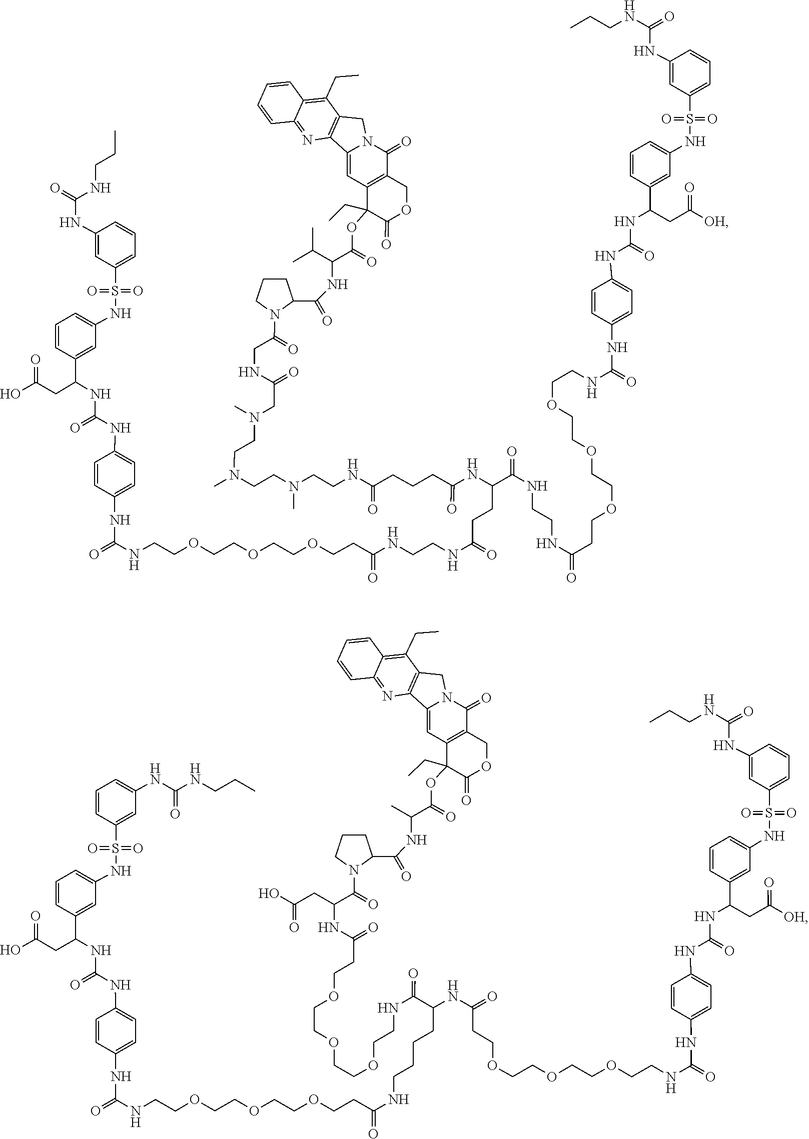

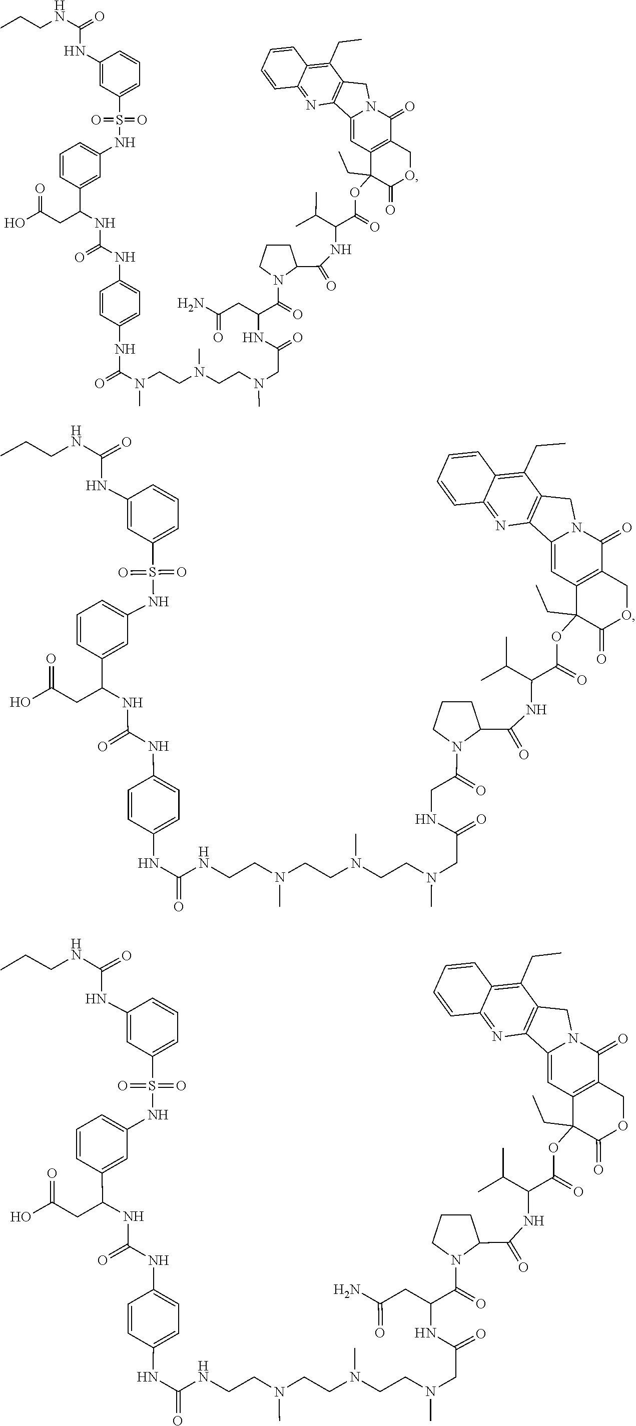

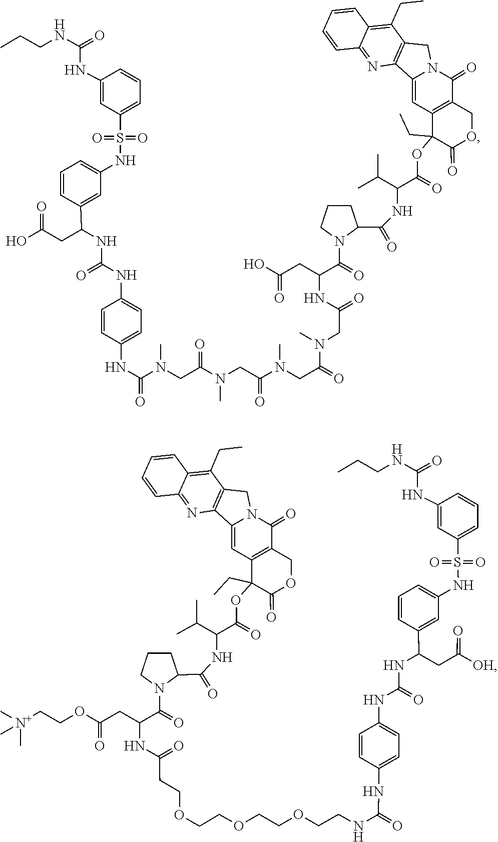 [SMILES: CCCNC(=O)Nc1cccc(S(=O)(=O)Nc2cccc(C(CC(=O)O)NC(=O)Nc3ccc(NC(=O)N(C)CC(=O)N(C)CC(=O)N(C)CC(=O)N(C)CC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCC[N+](C)(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]

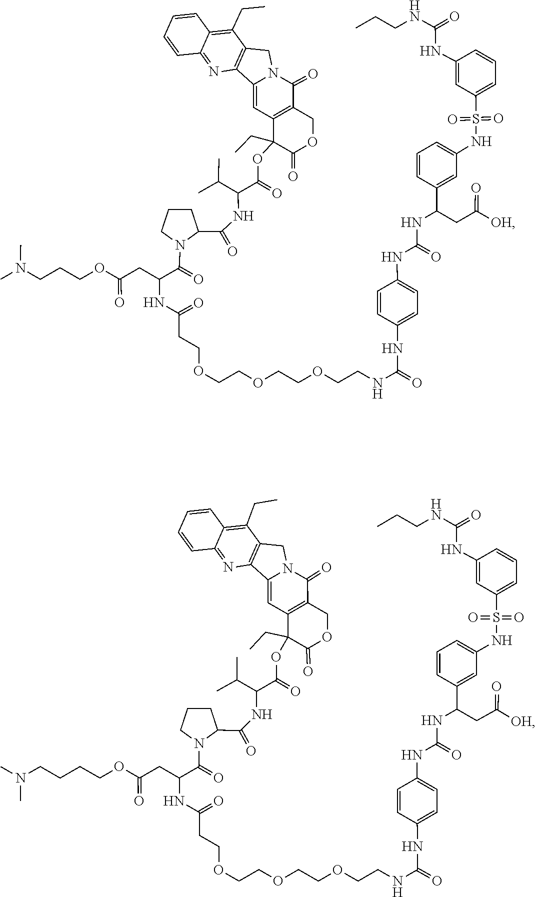 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCCN(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCCCN(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]

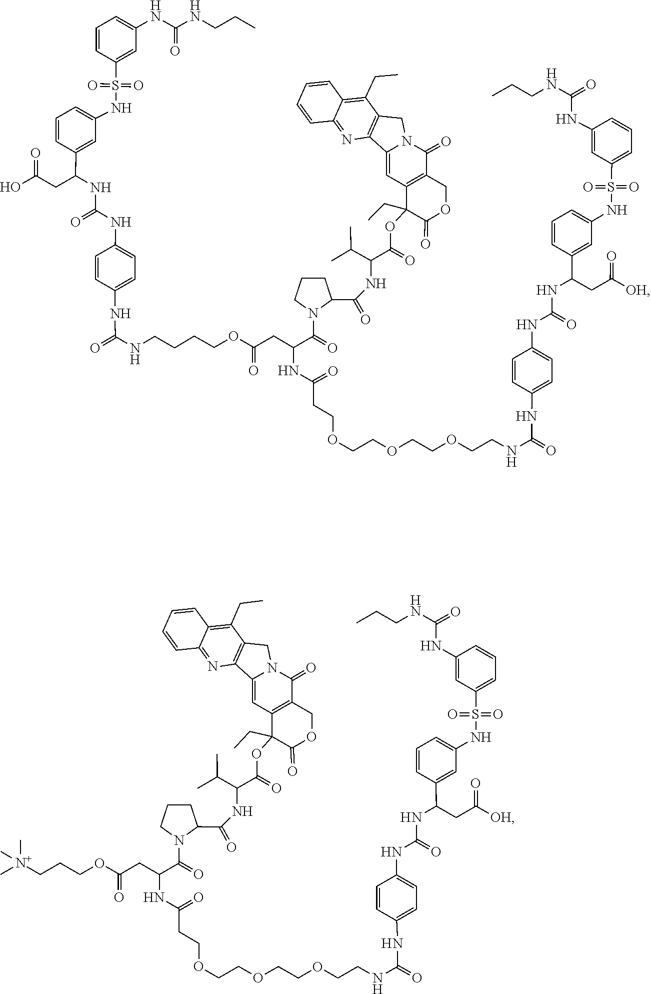 [SMILES: CCCNC(=O)Nc1cccc(S(=O)(=O)Nc2cccc(C(CC(=O)O)NC(=O)Nc3ccc(NC(=O)NCCCCOC(=O)CC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCC[N+](C)(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]

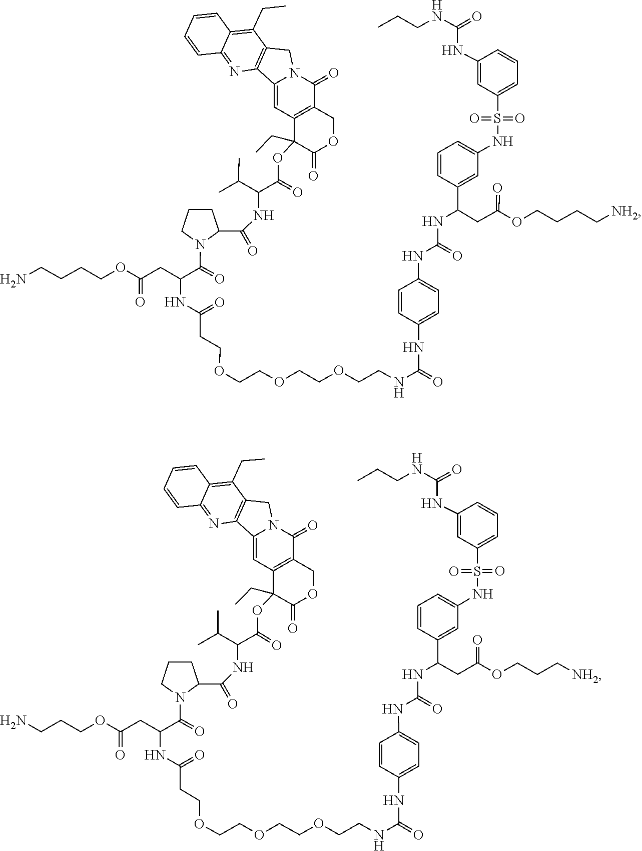 [SMILES: CCCNC(=O)Nc1cccc(S(=O)(=O)Nc2cccc(C(CC(=O)OCCCCN)NC(=O)Nc3ccc(NC(=O)NCCOCCOCCOCCC(=O)NC(CC(=O)OCCCCN)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)OCCCN)NC(=O)Nc3ccc(NC(=O)NCCOCCOCCOCCC(=O)NC(CC(=O)OCCCN)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]

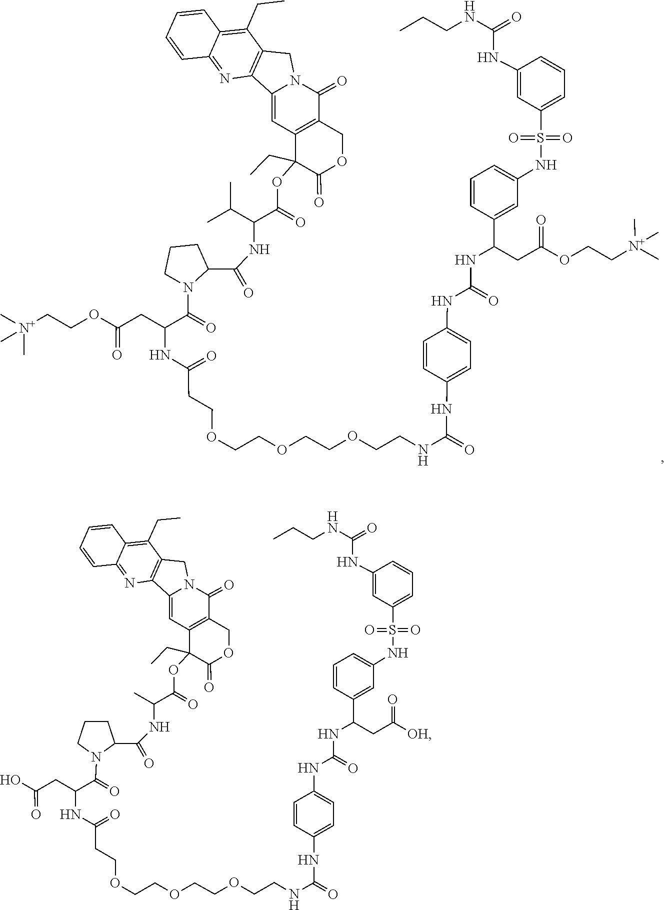

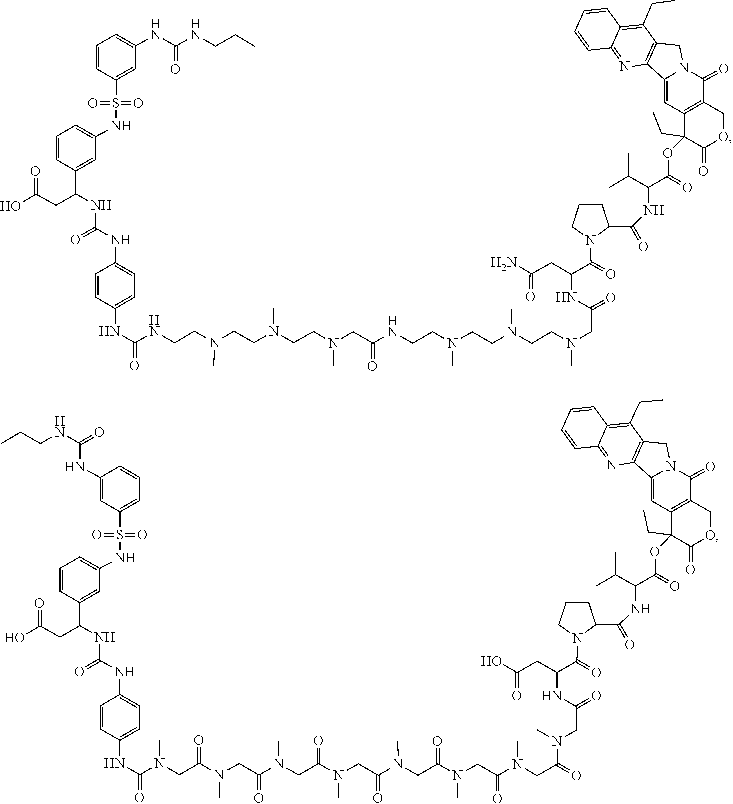 [SMILES: CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)N(C)CC(=O)N(C)CC(=O)N(C)CC(=O)N(C)CC(=O)N(C)CC(=O)N(C)CC(=O)N(C)CC(=O)N(C)CC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]

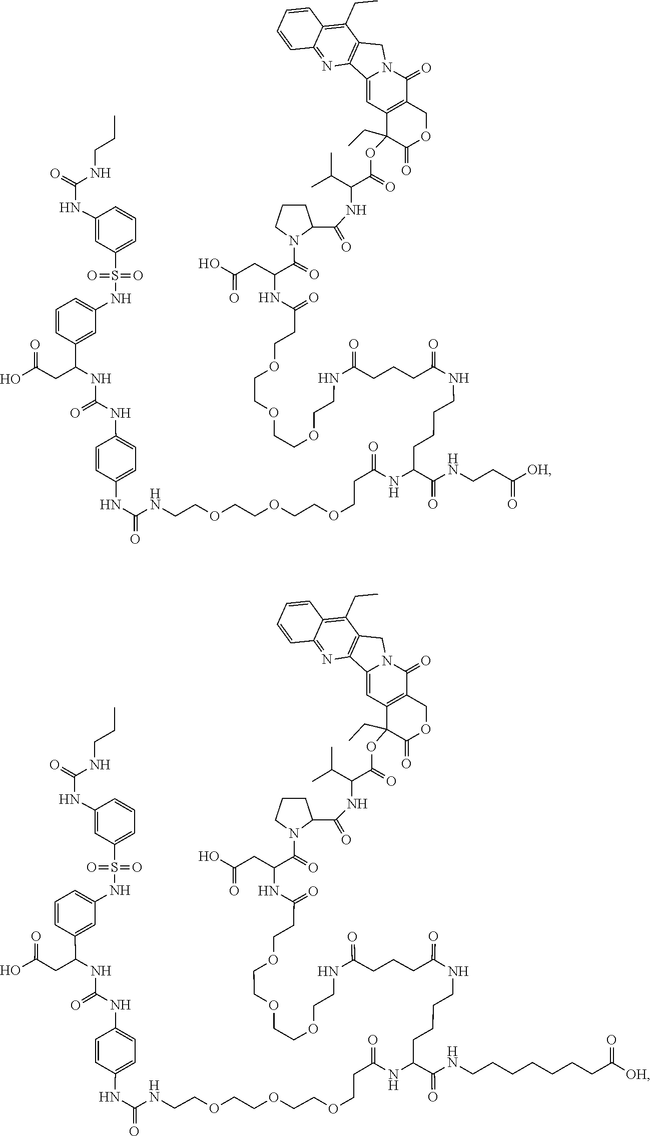 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CCCCNC(=O)CCCC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)C(=O)NCCC(=O)O)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(CCCCNC(=O)CCCC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)C(=O)NCCCCCCCC(=O)O)cc3)c2)c1]

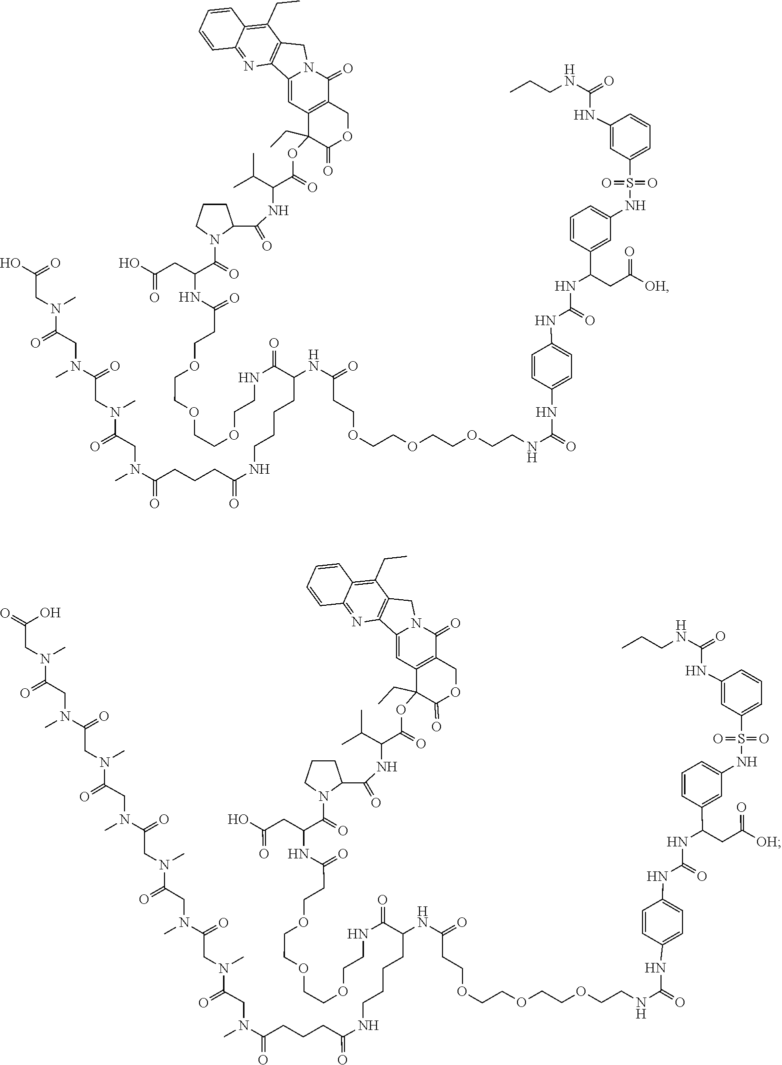

or a pharmaceutically acceptable salt thereof, or a stereoisomer or mixture of stereoisomers thereof.

In some embodiments, provided herein is a compound having a structure:

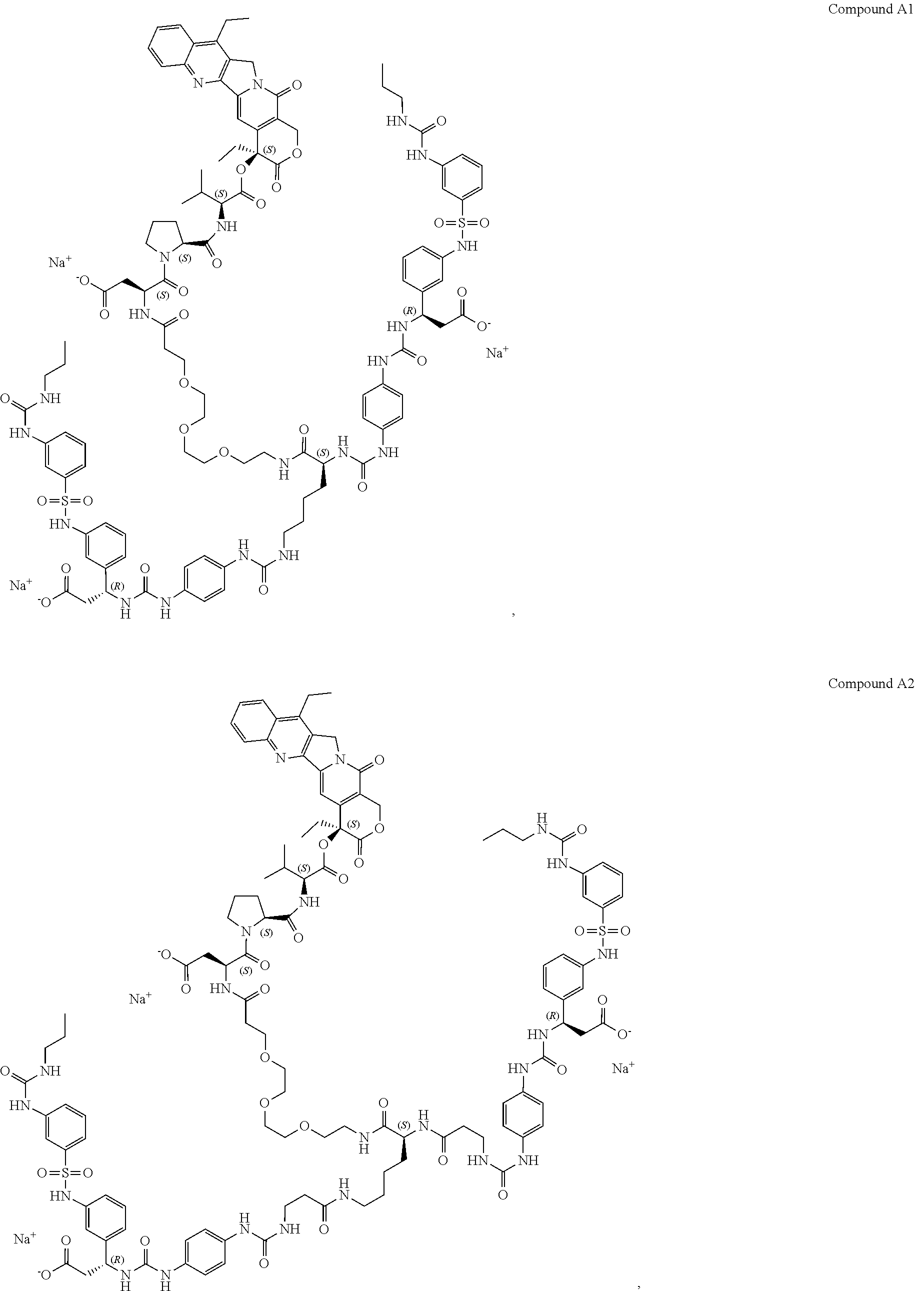

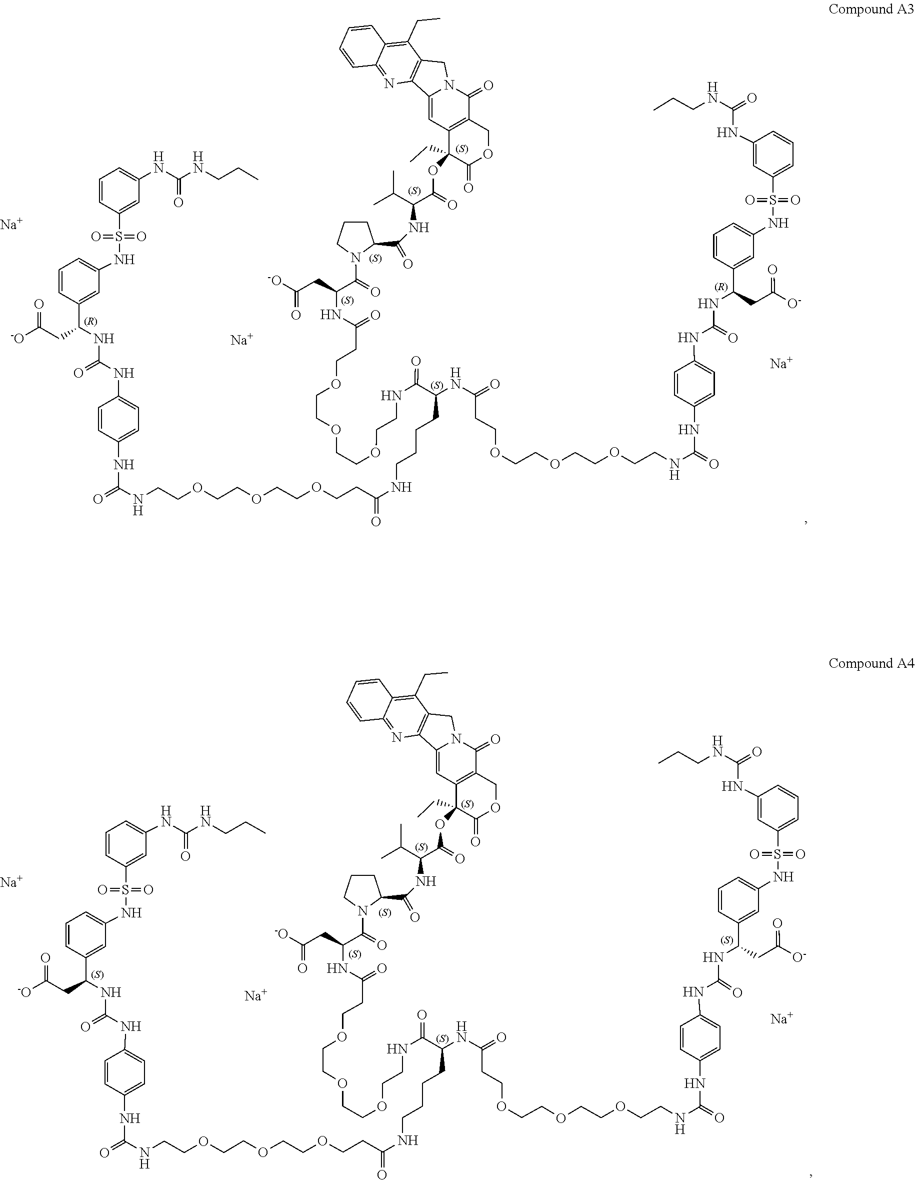

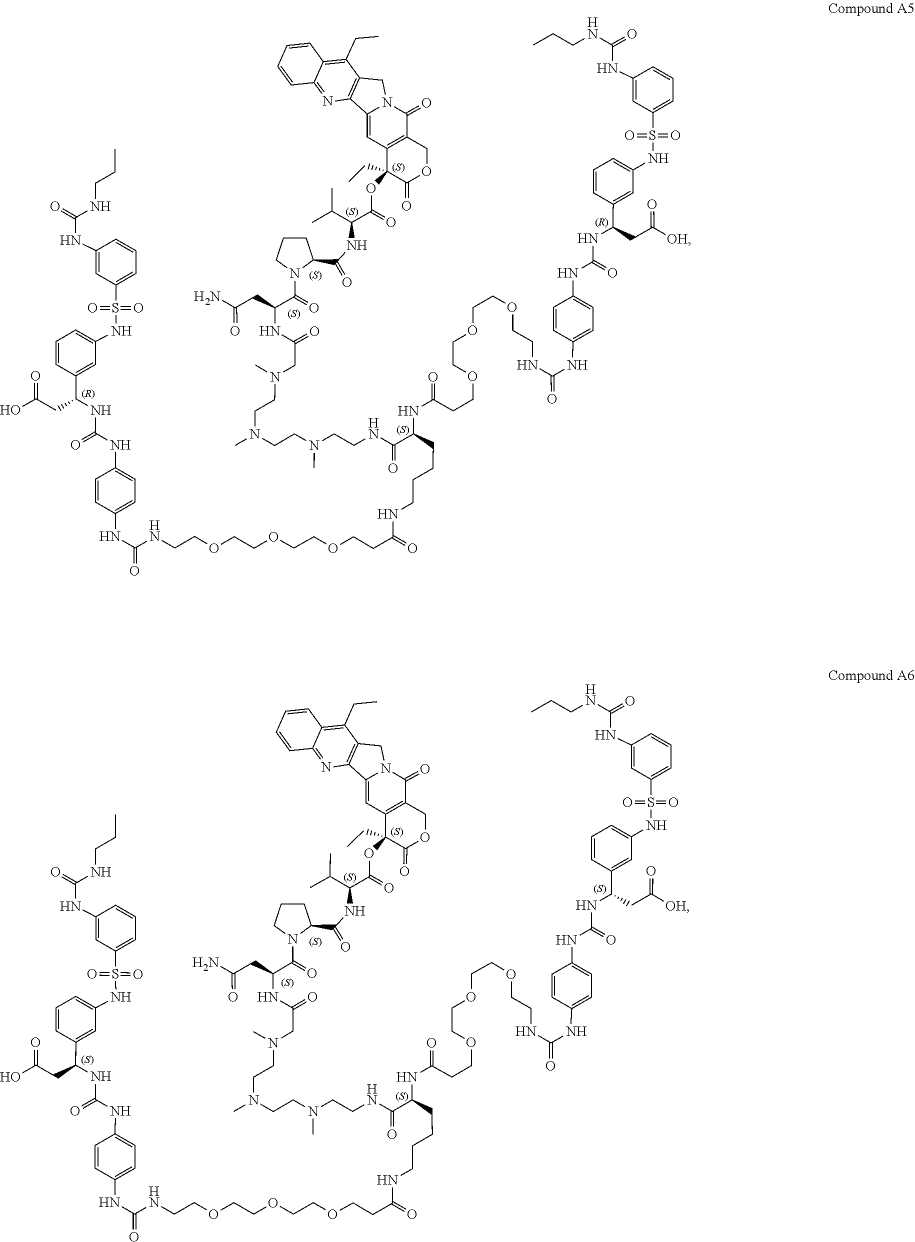

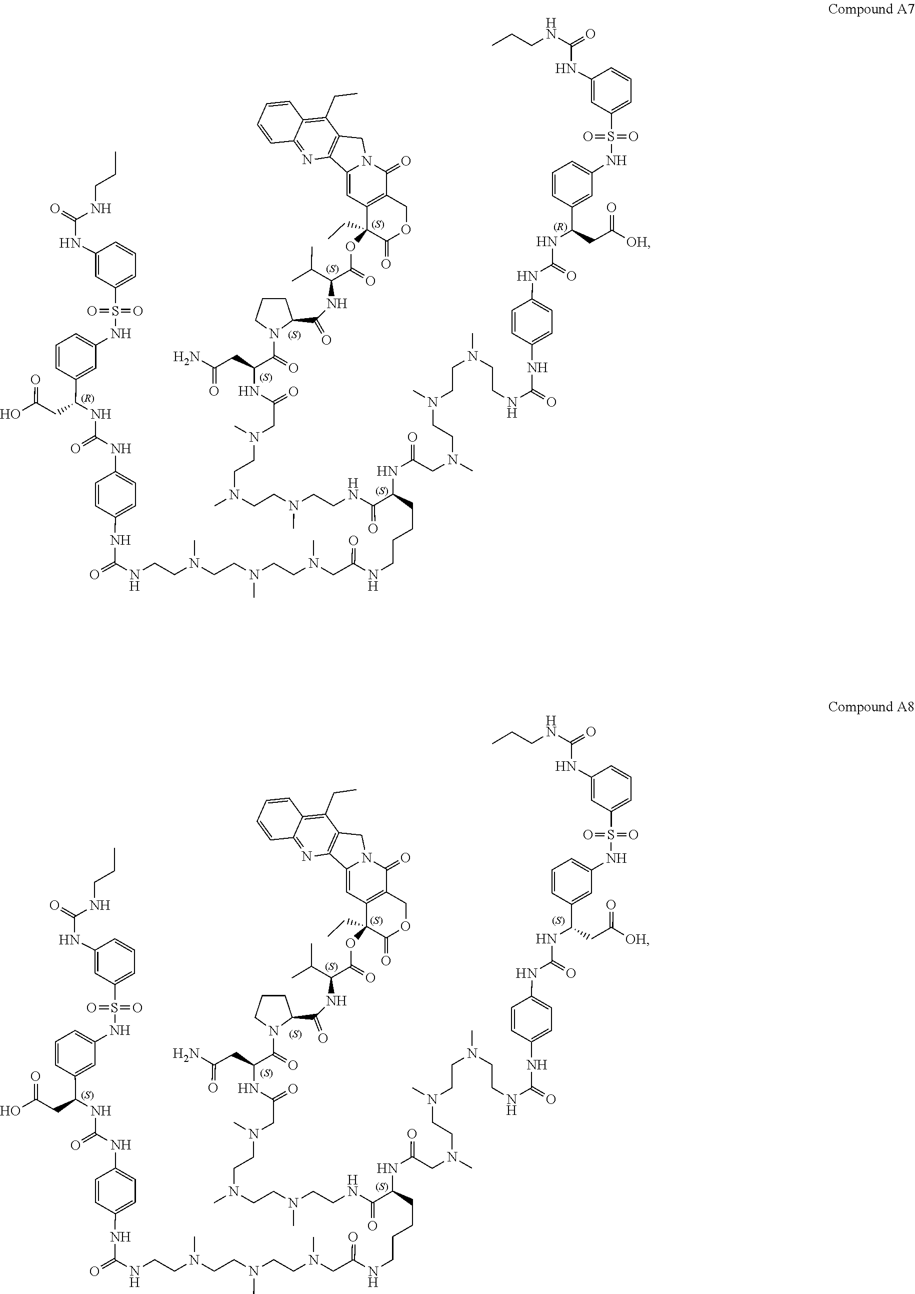

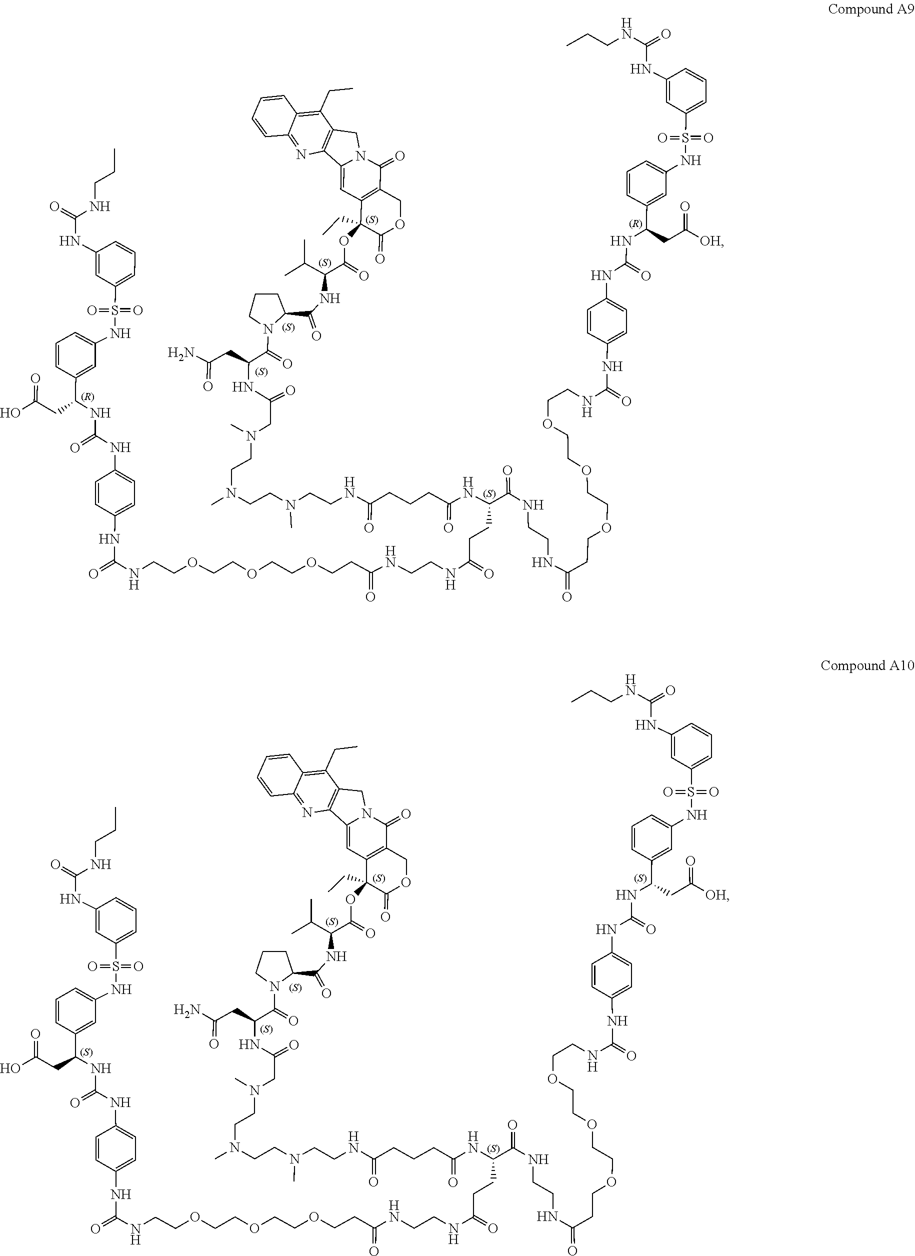

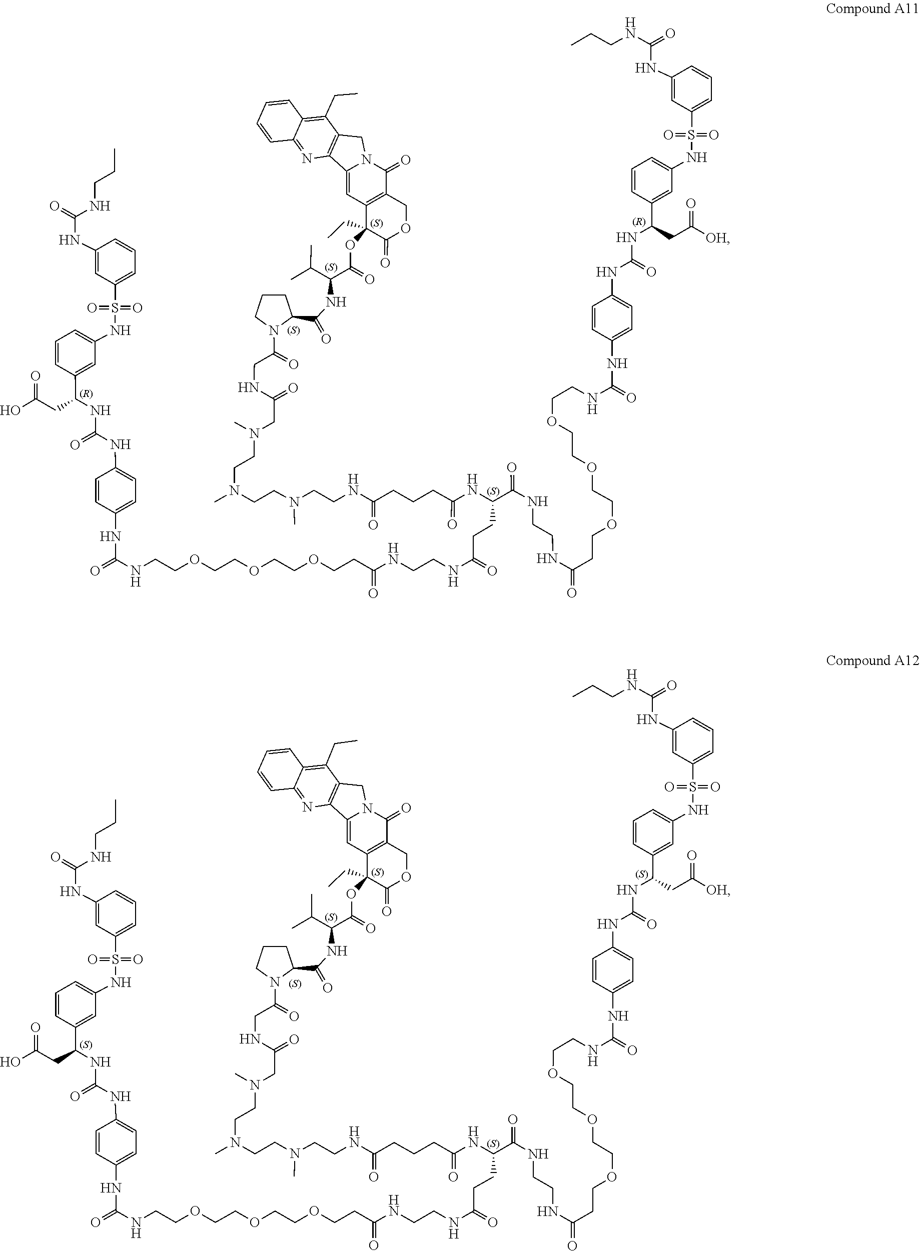

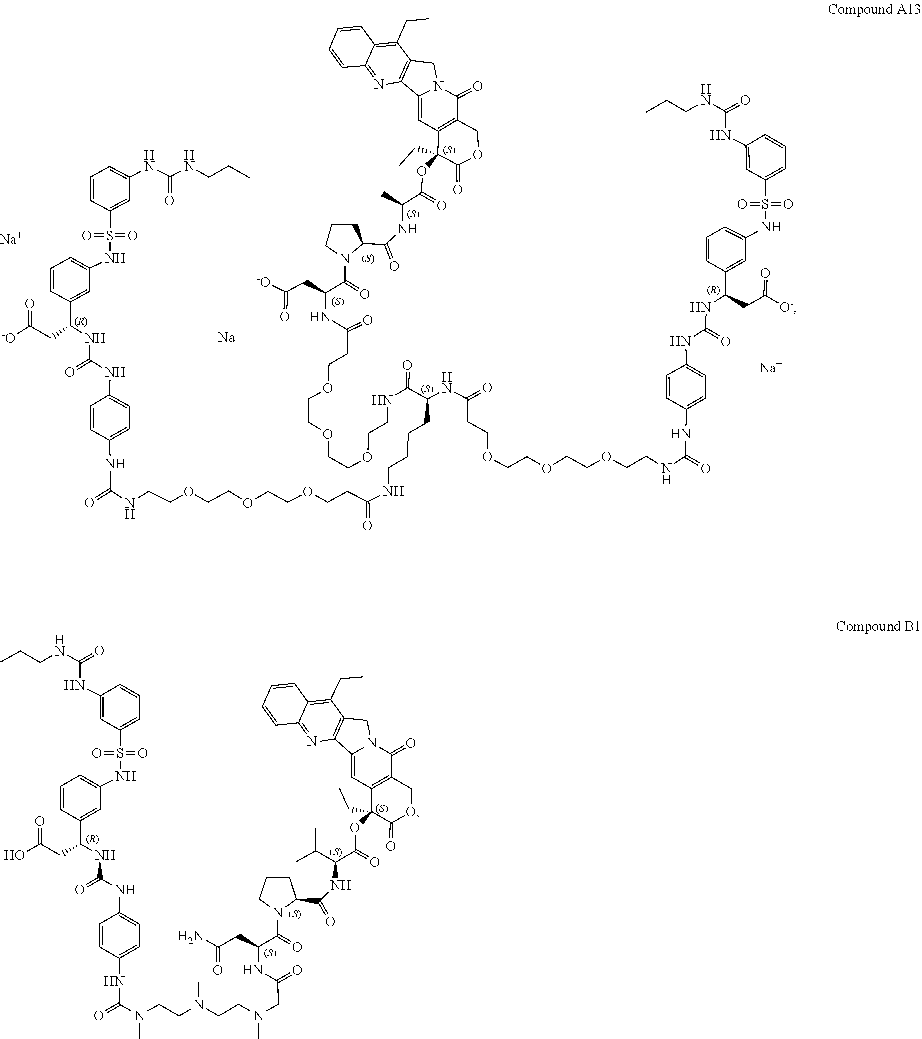

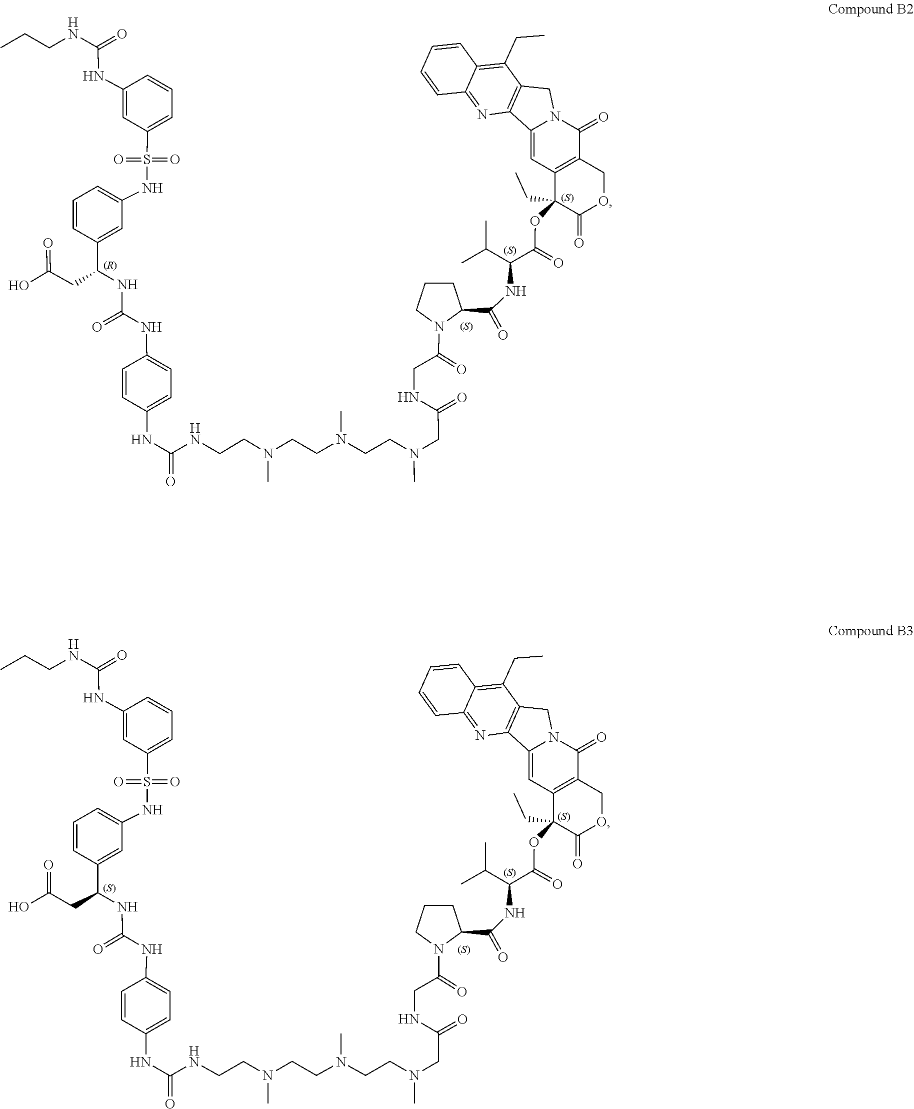

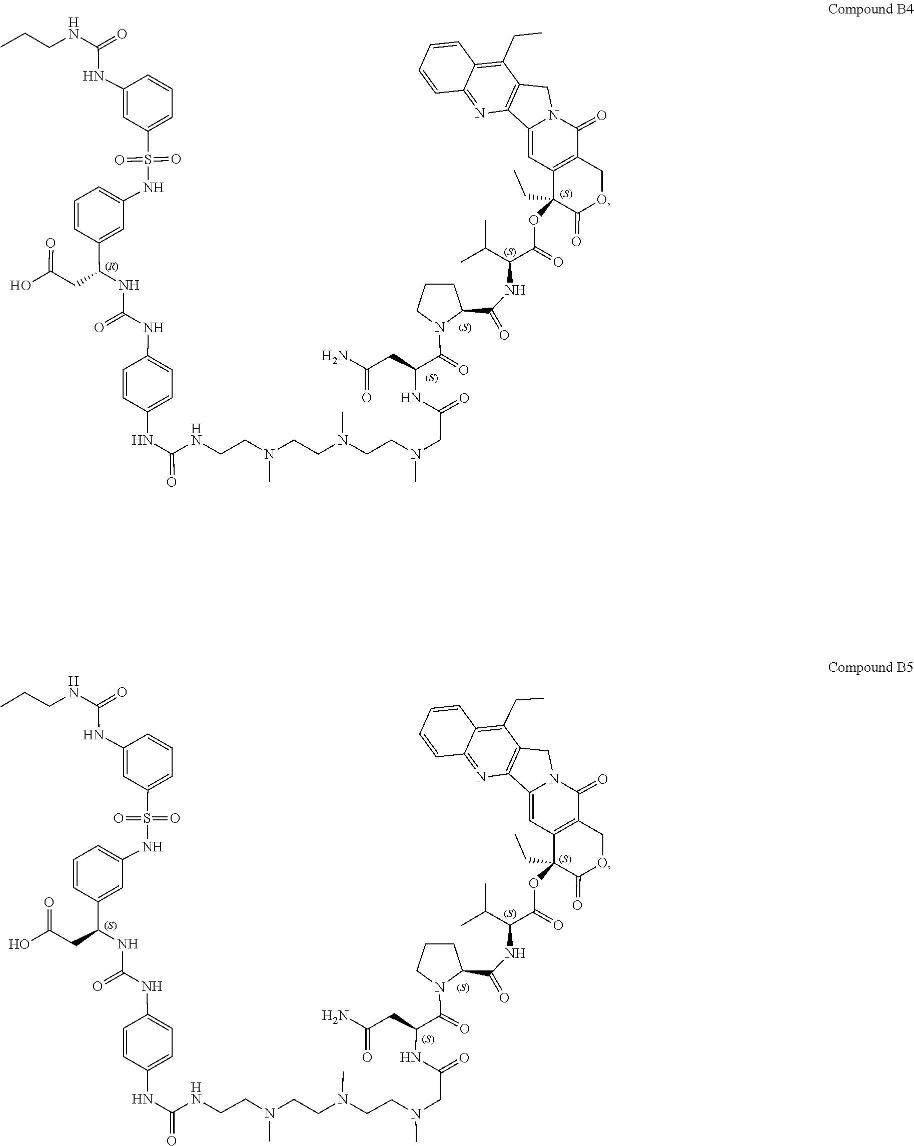

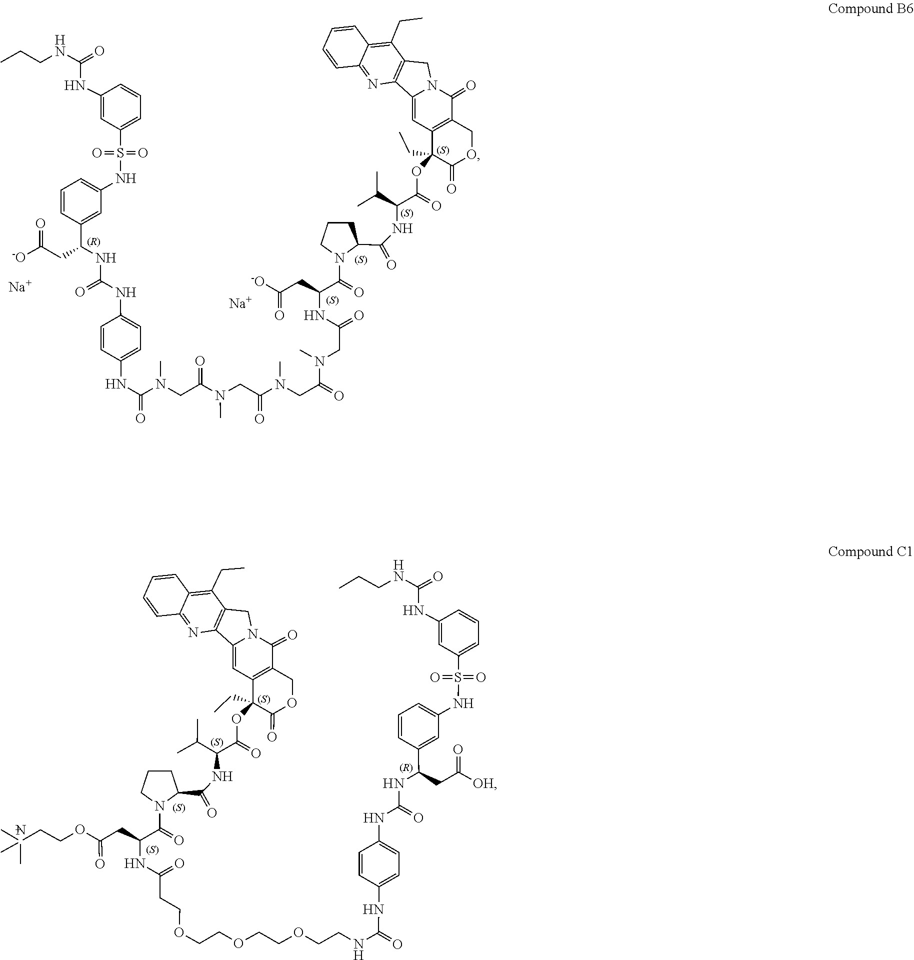

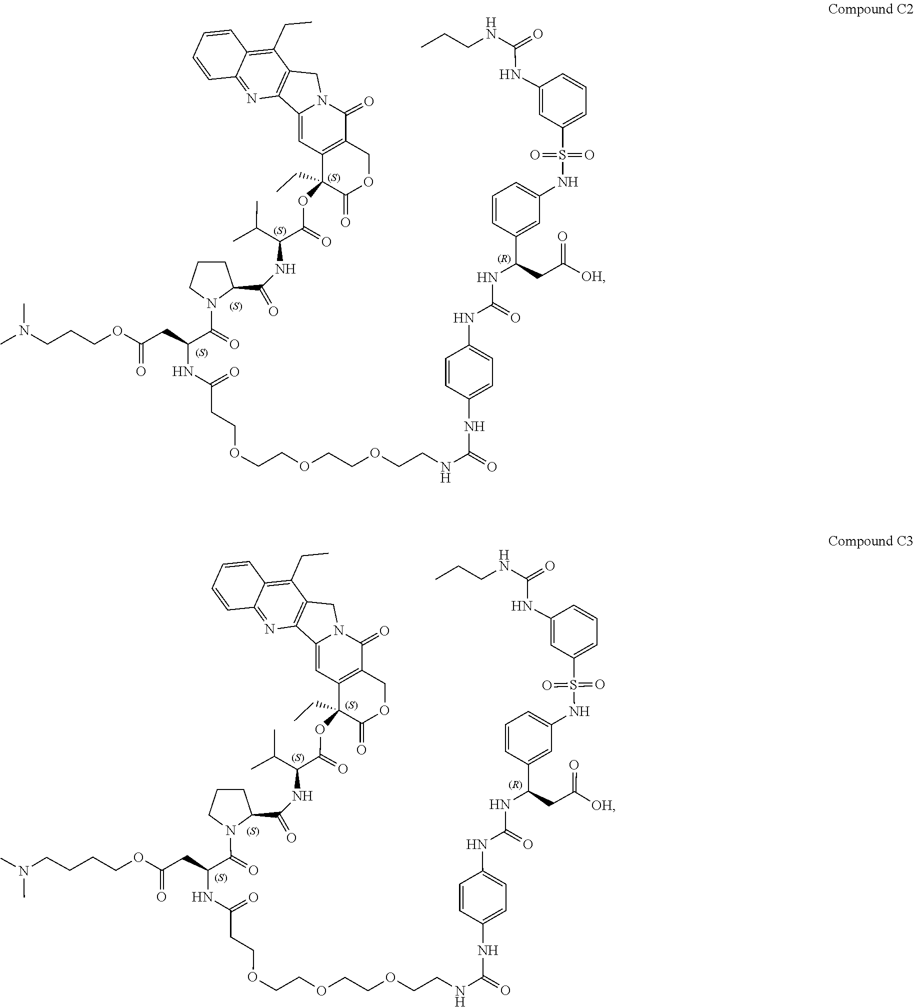

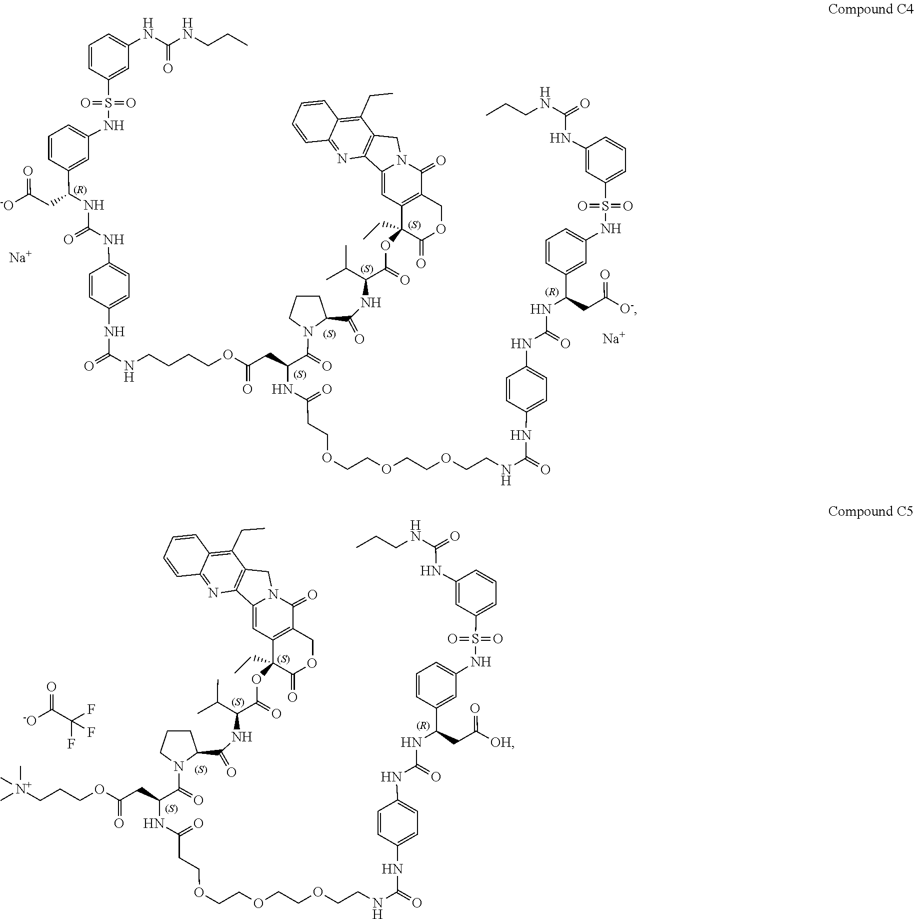

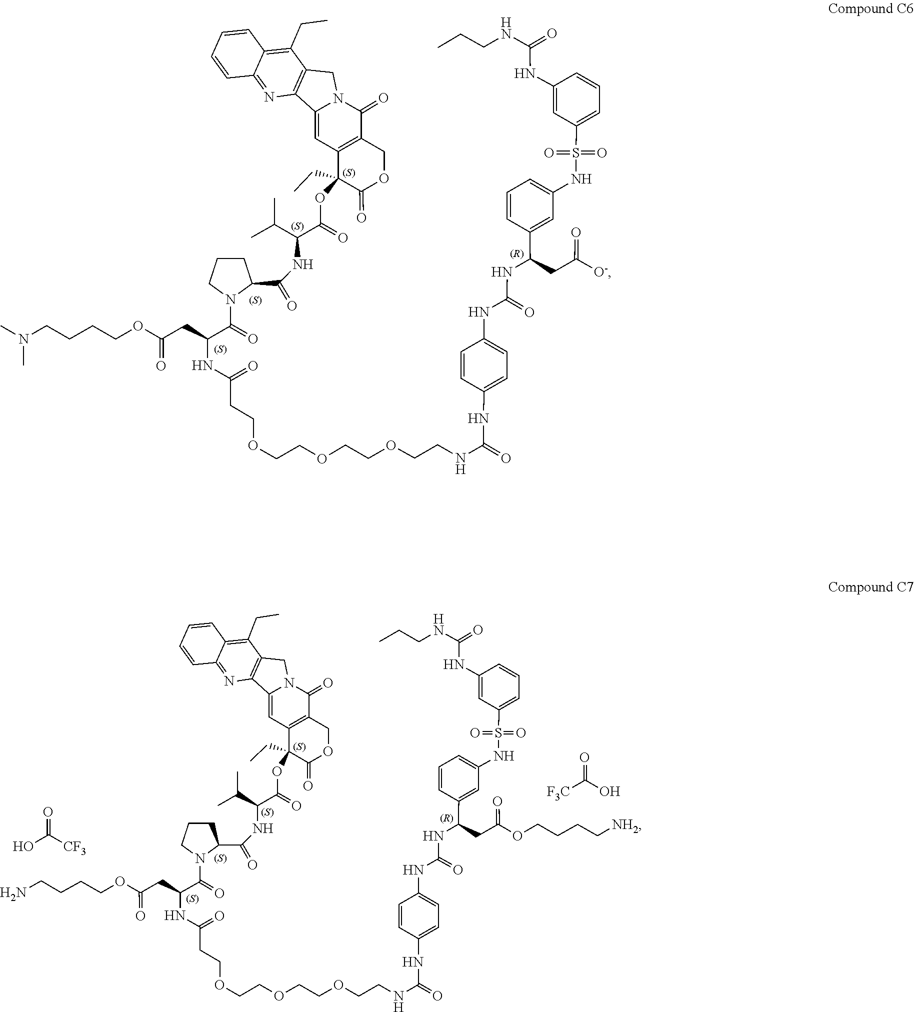

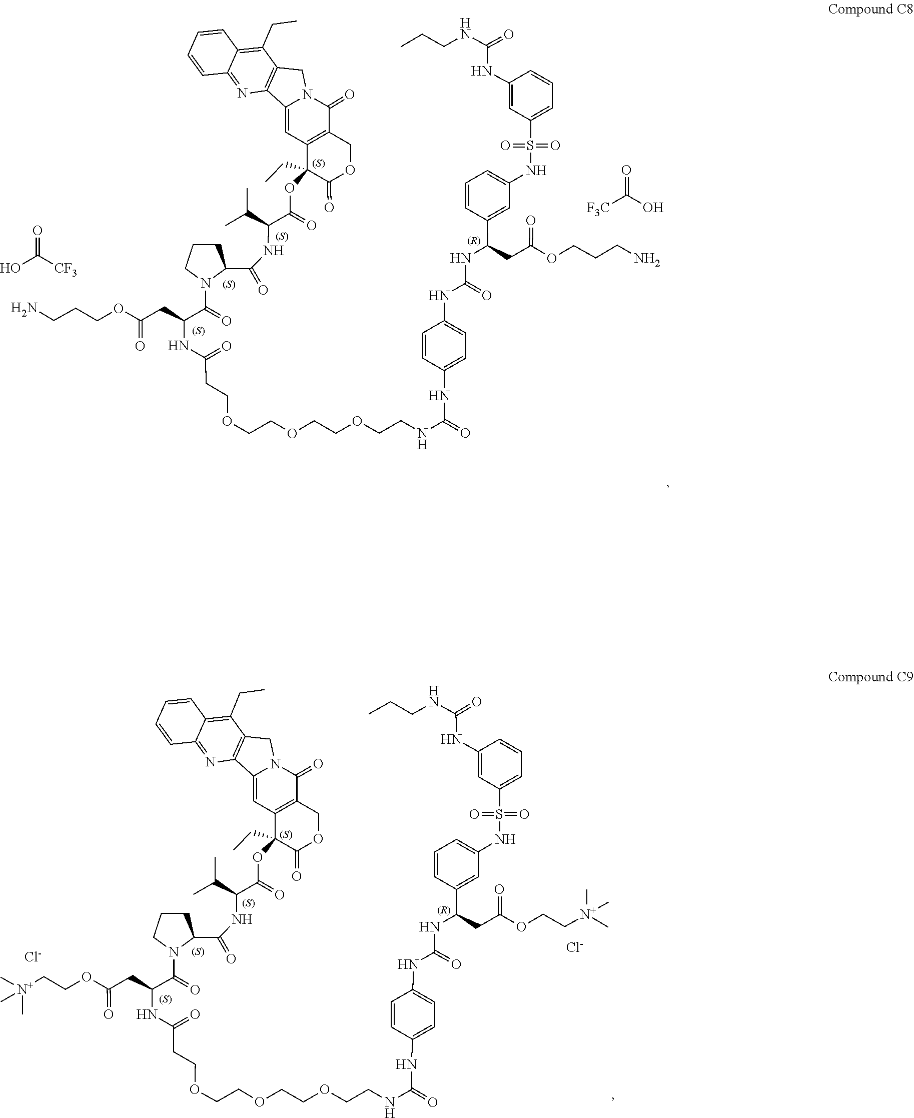

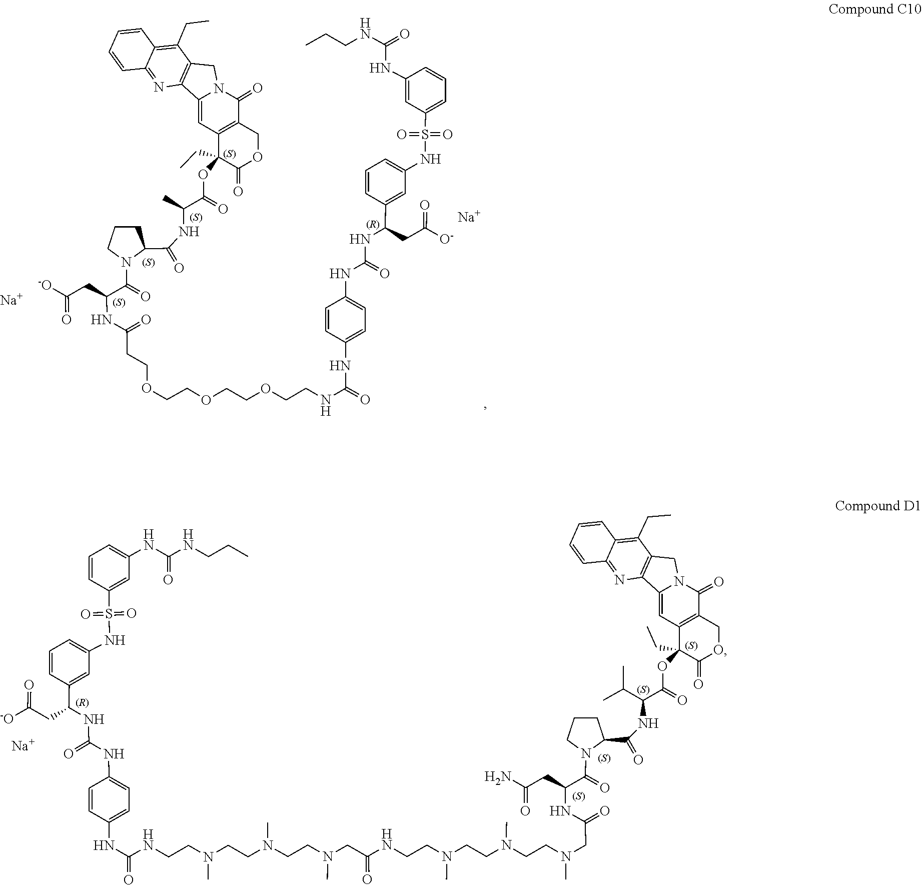

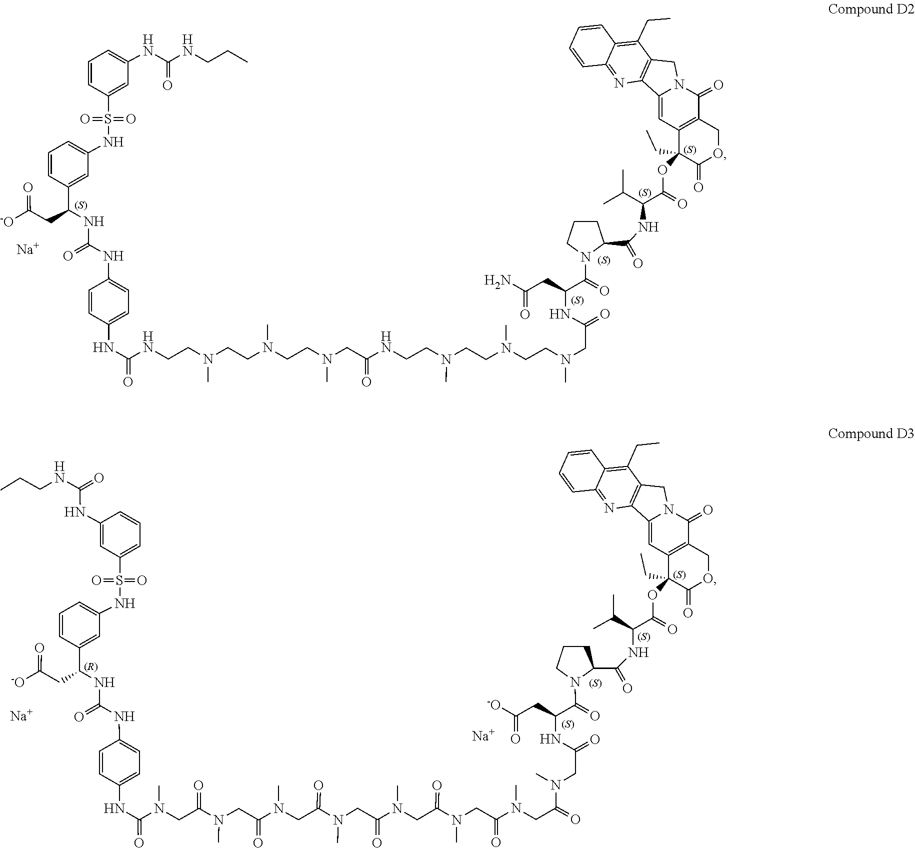

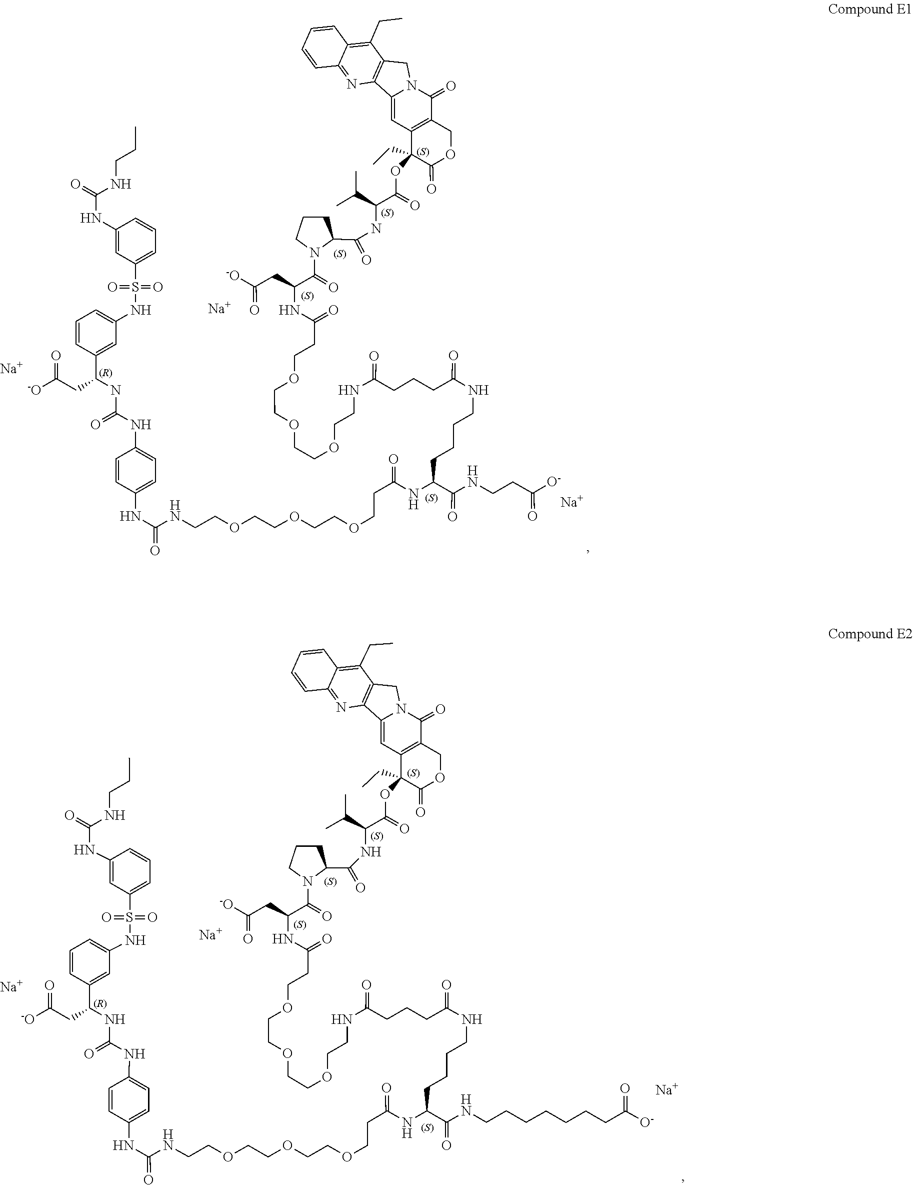

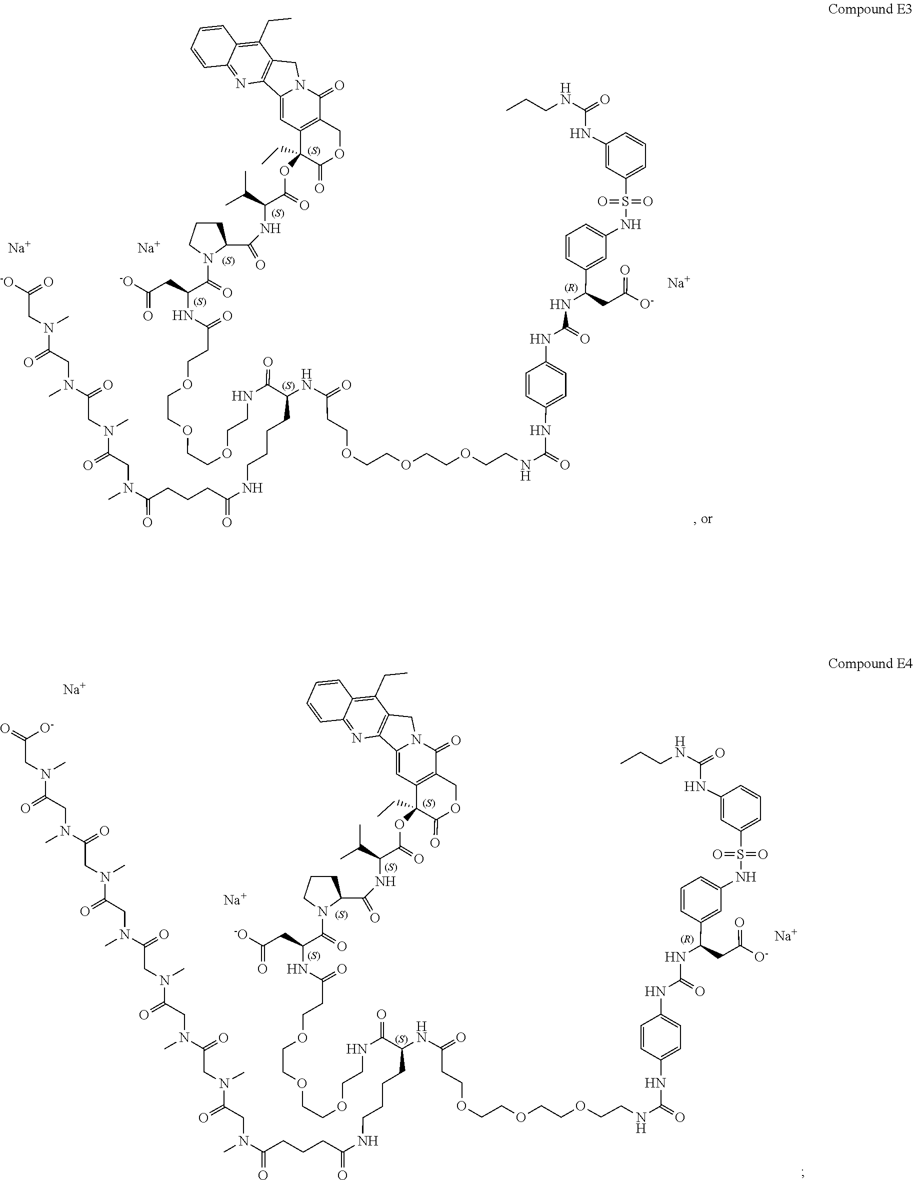

or a pharmaceutically acceptable salt thereof, or a stereoisomer or mixture of stereoisomers thereof.

In an aspect, provided herein is a compound, or a pharmaceutically acceptable salt thereof; or a stereoisomer or mixture of stereoisomers thereof, having a structure represented by any one of Formulae (01)-(05):

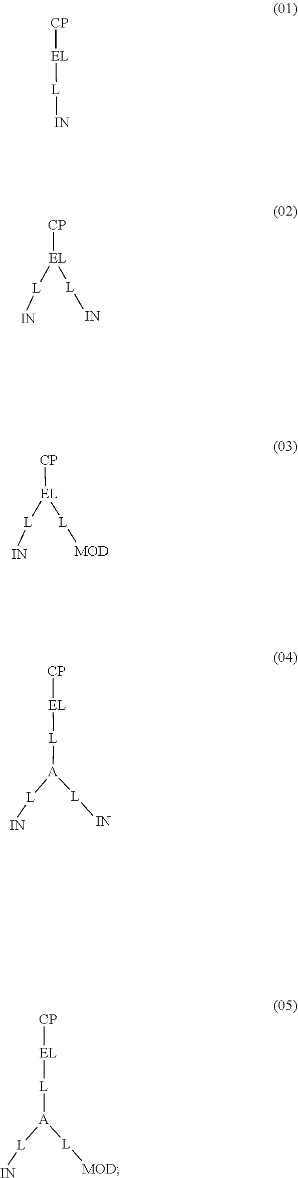

wherein:
CP is a cytotoxic or cytostatic group;
EL is an enzymatically-cleavable linker;
L is a stable linker;
A is a branching group;
IN is an integrin binding group; and
MOD is a physicochemical or pharmacokinetic modulator group.

In some embodiments, provided herein is a compound of Formula (01), wherein L is -L⁴-, -L⁵-, or -L⁷- as defined herein, or is the group -L¹A¹(L²-)(L³-), as defined herein. In some embodiments, the compound is of Formula (01), and L is L⁴, L⁵, or L⁷. In some embodiments, the compound is of Formula (02), wherein a first L is L⁴, L⁵, or L⁷, and a second L is L⁶. In some embodiments, the compound is of Formula (03), wherein a first L is L⁴, L⁵, or L⁷, and a second L is L⁶. In some embodiments, the compound is of Formula (03), wherein -L-MOD is -L⁶-MOD, and -L-IN is L⁴-IN, L⁵-IN, or L⁷-IN. In some embodiments, the compound of Formula (01), Formula (02), or Formula (03) is a compound of Formula (I-C), Formula (I-C1), or Formula (I-C2). In some embodiments, the compound of Formula (04) is a compound of Formula (I-A), Formula (I-A′), Formula (II-A), Formula (II-A′), Formula (III-A), Formula (III-A′), wherein each L corresponds to L¹, L², and L³ as defined herein, and A corresponds to A¹ as defined herein. In some embodiments, the compound of Formula (05) is a compound of Formula (I-A) or Formula (II-A), wherein one group “IN” is replaced with a group “MOD.” In some embodiments, the compound of Formula (05) is a compound of Formula (I-E), Formula (II-E), or Formula (III-E). In some embodiments, MOD is —OH, —COOH, —NH₂, —N(CH₃)₂, —N(CH₃)₃⁺, or —N(═NH)NH₂, or a salt thereof. In some embodiments, MOD is —OH or —COOH. In some embodiments, L is alkyl or aminoalkyl. In some embodiments, L is a polyamide (e.g., a polypeptide (e.g., a polyglycine)). In some embodiments, L is a poly-N(Me)Gly linker. In some embodiments, L is a PEG linker.

In some embodiments, each L is a stable linker having a structure represented by the formula:

—(CO)ₘ—(CH₂)ₙ—(OC₂₋₆ alkyl)ₒ-(NH)ₚ—(CO)_{q}—; (i)

—(CO)ᵣ—(CH₂)ₛ—(NR¹⁰C₂₋₆ alkyl)ₜ-(NR¹¹)ᵤ—(CO)ᵥ—; (ii)

—(CO)ᵣ—(CH₂)ₛ—(NR¹⁰C(O)C₁₋₆ alkyl)ₜ-(NR¹¹)ᵤ—(CO)ᵥ—; or (iii)

—(CO)ₘ—(CH₂)ₙ—(NH)ₚ—(CO)_{q}—; (iv)
wherein:
R¹⁰ is hydrogen or C₁₋₃ alkyl;
R¹¹ is hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1; and
v is 0 or 1.
   Spacers (L¹, L², L³, L⁴, L⁵, and L⁶)

Provided herein are compounds having linkers or spacers (used interchangeably) which serve to create physical space between one or more elements of the compound or conjugate. In some embodiments, the linkers or spacers provide additional utility (e.g., functional linkers). For example, in some embodiments, a linker is provided with a particular length that enables enhanced cleavage of an adjacent enzymatically cleavable moiety (e.g., EL). In some embodiments, a linker is provided with a particular length that reduces steric hinderance and/or enables greater target binding. In some embodiments, a linker is provided in a particular length such that an integrin binder can bind an integrin receptor (e.g., an αᵥβ3 integrin receptor) with potency (i.e., IC₅₀) that is 1.0⁻⁹ M or lower (e.g., 9E⁻¹⁰, 8E⁻¹⁰, 7E⁻¹⁰, 6E⁻¹⁰, 5E⁻¹⁰, 4E⁻¹⁰, 3E⁻¹⁰, 2E⁻¹⁰, 1E⁻¹⁰, or lower). In some embodiments, a linker is provided in a particular length such that an integrin binder can bind an integrin receptor (e.g., an αᵥβ3 integrin receptor) with potency (i.e., IC₅₀) that is 1.0⁻¹⁰ M or lower (e.g., 9E⁻¹¹, 8E⁻¹¹, 7E⁻¹¹, 6E⁻¹¹, 5E⁻¹¹, 4E⁻¹¹, 3E⁻¹¹, 2E⁻¹¹, 1E⁻¹¹, or lower). In some embodiments, a linker is provided in a particular length such that an integrin binder can bind an integrin receptor (e.g., an αᵥβ₃ integrin receptor) with potency (i.e., IC₅₀) that is 1.0⁻¹¹ M or lower (e.g., 9E⁻¹², 8E⁻¹², 7E⁻¹², 6E⁻¹², 5E⁻¹², 4E⁻¹¹, 3E⁻¹², 2E⁻¹², 1E⁻¹², or lower).

In some embodiments, provided herein is a compound comprising a linker (e.g., L¹, L², L³, L⁴, L⁵, L⁶, and/or L⁷), wherein the linker enhances retention of the compound within a tumor microenvironment. In some embodiments, a linker disclosed herein (e.g., a polyamine or polyamide linker) increases the tumor to plasma ratio of a compound, compared to a reference compound comprising an alkyl or PEG linker. In some embodiments, provided herein is a compound comprising a trivalent linker (A¹) and two linker-binder groups (L²IN, L³IN), or a linker-binder and linker-MOD group (L²IN)(L³MOD), wherein the compound comprising the L³IN or L³MOD group has an increased half-life and/or increased AUC, compared to a compound comprising a bivalent linker. In some embodiments, provided herein is a compound comprising a trivalent linker and two integrin binding groups, wherein the compound comprising a trivalent linker and two integrin-bonding groups has an increased half-life and/or increased AUC, compared to a compound comprising a bivalent linker.

In some embodiments, a linker is a branched or linear chain of atoms selected from C, N, O, or S (each of which being substituted with hydrogens or bonds so as to fulfill standard valence). In some embodiments, a linker is a carbonyl (—C(O)—). In some embodiments, a linker contains six or less (non-hydrogen) atoms. In some embodiments, a linker contains about 10 to about 20 (non-hydrogen) atoms. In some embodiments, a linker contains about 10 to about 20 (non-hydrogen) atoms arranged in a linear chain. In some embodiments, a linker contains about 20 to about 30 (non-hydrogen) atoms. In some embodiments, a linker contains about 20 to about 30 (non-hydrogen) atoms arranged in a linear chain. In some embodiments, a linker contains about 30 to about 40 (non-hydrogen) atoms. In some embodiments, a linker contains about 30 to about 40 (non-hydrogen) atoms arranged in a linear chain. In some embodiments, multiple linkers are present, each of which having a different length. In some embodiments, a linker contains cyclic or branched moieties. In some embodiments, a linker is substituted with one or more alkyl, oxo, amino, or amide groups.

In some embodiments, each of L¹, L², L³, L⁴, L⁵, L⁶, and L⁷ contains or is terminally substituted with one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—).

In some embodiments, each of L¹, L², L³, L⁴, L⁵, L⁶, and L⁷ is a substituted or unsubstituted C₂₋₂₀ alkyl chain that is optionally interrupted one or more times by groups selected from —O—, —NH—, —N(CH₃)—, —C(O)—, —C(O)NH—, —C(O)N(CH₃)—, —C(O)O—, —NHC(O)—, —N(CH₃)C(O)—, —NHC(O)NH—, —S—, —S(O)—, —S(O)₂—, or any combination thereof. In some embodiments, L¹, L², L³, L⁴, L⁵, L⁶, and/or L⁷ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —NHS(O)₂NH—, —NHS(O)₂NHC(O)—, —NHS(O)₂NHC(O)O—, —O—, —S—, —S(O)—, —S(O)₂—, —S(O)₂NH—, —S(O)₂NHC(O)—, —S(O)₂NHC(O)NH—, —S(O)₂NHC(O)O—, carbocyclyl, heterocyclyl, aralkyl, heteroaralkyl, or any combination thereof. In some embodiments, L¹, L², L³, L⁴, L⁵, L⁶, and/or L⁷ is a linker disclosed in WO2016207089, which is incorporated by reference in its entirety. In some embodiments, a linker comprises a C₁₋₃₀ alkyl or heteroalkyl group (optionally substituted), which is optionally interrupted by an aryl or heteroaryl group (e.g., a triazole, a dibenzocyclooctyne, or a derivative thereof). For example, a linker may comprise a dibenzylcyclooctyne (DBCO) derivative such as a DBCO NHS ester, or a chemical group formed therefrom. For example, an interrupting group may include:

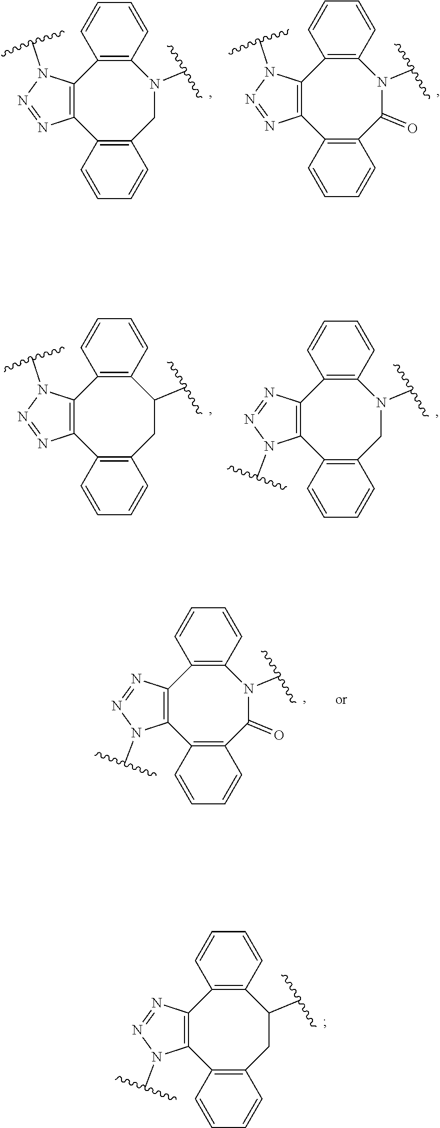 [SMILES: *N1Cc2ccccc2-c2nnn(*)c2-c2ccccc21 ; *N1C(=O)c2ccccc2-c2nnn(*)c2-c2ccccc21 ; *C1Cc2ccccc2-c2nnn(*)c2-c2ccccc21 ; *N1Cc2ccccc2-c2c(nnn2*)-c2ccccc21 ; *N1C(=O)c2ccccc2-c2c(nnn2*)-c2ccccc21 ; *C1Cc2ccccc2-c2c(nnn2*)-c2ccccc21]

or a derivative thereof, wherein the interrupting group is substituted on each end to form a linker (e.g., L¹, L², L³, L⁴, L⁵, L⁶, and/or L⁷). In some embodiments, an interrupting group may include:
—NHS(O)₂NH—, —NHS(O)₂NHC(O)—, —NHS(O)₂NHC(O)O—, or

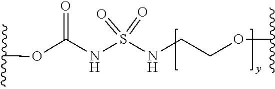

(wherein y is 1-20), or any combination thereof. In some embodiments, L¹, L², L³, L⁴, L⁵, L⁶, and/or L⁷ is a sulfamide linker, comprising one or more (e.g., one, two, three, or four) simple spacers defined herein, optionally interrupted (i.e., conjoined) by a sulfamide group (e.g., —NHS(O)₂NH—, —NHS(O)₂NHC(O)—, or —NHS(O)₂NHC(O)O—). In some embodiments, L¹, L², L³, L⁴, L⁵, L⁶, and/or L⁷ comprises a sulfamide group (—NHS(O)₂NH—). In some embodiments, Lt, L², L³, L⁴, L⁵, L⁶, and/or L⁷ is a sulfamide linker, consisting of one or more (e.g., one, two, three, or four) of the following groups: —NHS(O)₂NH—, —NHS(O)₂NHC(O)—, —NHS(O)₂NHC(O)O—, and

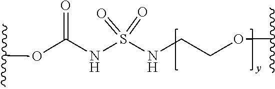

(wherein y is 1-20), and wherein the sulfamide linker is optionally conjoined to an adjacent group via a spanner (as defined herein).

In some embodiments, a linker is ionized in a biological system (e.g., within an acidic tumor microenvironment). In some embodiments, an ionized or partially charged (e.g., partially positive or partially negative) moiety or moieties within a linker enhance localization of the compound in a preferred locale (e.g., extracellularly, within a tumor microenvironment).

In some embodiments, a linker (e.g., one or more of L¹, L², L³, L⁴, L⁵, L⁶, and L⁷) is an optionally substituted polyamine linker or an optionally substituted polyamide linker.

In some embodiments, a polyamine or polyamide linker is a functional linker. In some embodiments, the functional linker is a polyamide or a sulfamide linker, wherein the functional linker increases retention of the compound within a tumor microenvironment (i.e., relative to an alkyl or PEG linker) and/or decreases liver exposure. In some embodiments, a functional linker (e.g., a polyamide linker) reduces immunogenic response to a compound containing said functional linker (i.e., relative to a PEG linker). In some embodiments, a polyamide or sulfamide linker disclosed herein improves one or more pharmacokinetic parameters. In some embodiments, a polyamide or sulfamide linker disclosed herein reduces liver toxicity, increase tumor-to-liver exposure, and/or increases liver clearance of a compound containing said functional linker. In some embodiments, a linker disclosed herein increases stability (i.e., reduces off-target release of a pa In some embodiments, biodistribution, AUC, and/or solubility are impacted (e.g., favorably) by a functional linker disclosed herein.

In some embodiments, the polyamine or polyamide linker of one or more of L¹, L², L³, L⁴, L⁵, L⁶, and L⁷ is substituted with one or more carbonyl groups.

In some embodiments, the polyamine linker of one or more of L¹, L², L³, L⁴, L⁵, L⁶, and L⁷ forms an aminium ion (or optionally multiple aminium ions) in an acidic tumor microenvironment.

In some embodiments, the polyamine or polyamide linker of one or more of L¹, L², L³, L⁴, L⁵, L⁶, and L⁷ is selectively retained in a tumor microenvironment.

In some embodiments, the present invention provides compounds having a structure of formulae disclosed herein, or a pharmaceutically acceptable salt thereof; or a stereoisomer or mixture of stereoisomers thereof; wherein each of L¹, L², L³, L⁴, and L⁶ is independently a bond, substituted or unsubstituted C₁₋₃₀ alkyl, or substituted or unsubstituted heteroalkyl. In some embodiments, each of L¹, L², L³, L⁴, and L⁶ is independently —C(O)—, substituted or unsubstituted C₂₋₃₀ alkyl, or substituted or unsubstituted heteroalkyl.

In some embodiments, each of L¹, L², L³, L⁴, and L⁶ is a substituted or unsubstituted C₂₋₂₀ alkyl chain that is optionally interrupted one or more times by groups, each independently selected from —O—, —S—, —NH—, —N(CH₃)—, —C(O)—, —C(O)NH—, —C(O)N(CH₃)—, —C(O)O—, —NHC(O)—, N(CH₃)C(O)—, or —NHC(O)NH—, or any combination thereof.

In some embodiments, each of L¹, L², L³, L⁴, and L⁶, contains (e.g., is terminally substituted with) one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—). In some embodiments, spacers such as L¹, L², L³, L⁴, and L⁶ contain one or more polymeric units selected from the group consisting of:

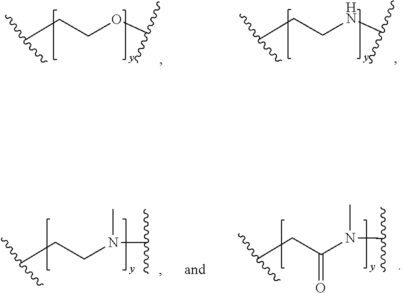

In some embodiments, the spacer comprises one to twenty polymeric units (i.e., y is 1-20). In some embodiments, y is between 2 and 6 (e.g., y is 2 to 6, 2 to 4, 2 or 3, 3 to 6, 3 or 4, etc.). In some embodiments, y is between 6 and 12 (e.g., y is 6 to 12, 6 to 10, 6 to 8, 8 to 12, 8 to 10, etc.). In some embodiments, y is 2, 3, or 4. In some embodiments, y is 8, 9, or 10.

In some embodiments, one or more of L¹, L², L³, L⁴, L⁵, and L⁶ is an optionally substituted polyamine linker or an optionally substituted polyamide linker. In some embodiments, a polyamine or polyamide linker of L¹, L², L³, L⁴, L⁵, and/or L⁶ forms an aminium ion within an acidic tumor microenvironment. In some embodiments, a polyamine or polyamide linker of L¹, L², L³, L⁴, L⁵, and/or L⁶ is selectively retained in a tumor microenvironment. In some embodiments, a polyamine or polyamide linker of L¹, L², L³, L⁴, L⁵, and/or L⁶ enhances retention of a conjugate in a tumor microenvironment. In some embodiments, a polyether linker of L¹, L², L³, L⁴, L⁵, and/or L⁶ enhances the aqueous solubility of a conjugate and/or reduces aggregation. In some embodiments, an extended linker provides a preferred distance or reduces steric hinderance between an integrin binder and a cytotoxic or cytostatic radical.

In some embodiments, a polyamide linker of L¹, L², L³, L⁴, L⁵, L⁶, and/or L⁷, (e.g., a polysarcosine linker) increases the tumor to plasma ratio of the compound comprising said linker, compared to a reference compound comprising an analogous alkyl or PEG linker.

In some embodiments, L¹, L², L³, L⁴, L⁵ and/or L⁶ is a simple spacer selected from the group consisting of
—O— —S—
—S(O)₂— —C(O)—
—C₁₋₃₀ alkyl-, —C(O)—C₁₋₃ alkyl
—C₁₋₃₀ alkyl-C(O)—, —C(O)—C₁₋₃₀ alkyl-C(O)—,
—C₁₋₃₀ alkyl-C(O)NH—, —C(O)—C₁₋₃₀ alkyl-C(O)NH—,
—C₁₋₃₀ alkyl-C(O)N(CH₃)—, —C(O)—C₁₋₃₀ alkyl-C(O)N(CH₃)—,
—C₁₋₃₀ alkyl-NH—, —C(O)—C₁₋₃₀ alkyl-NH—,
—C₁₋₃₀ alkyl-NHC(O)—, —C(O)—C₁₋₃₀ alkyl-NHC(O)—,
—C₁₋₃₀ alkyl-N(CH₃)—, —C(O)—C₁₋₃₀ alkyl-N(CH₃)—,
—C₁₋₃₀ alkyl-N(CH₃)C(O)—, —C(O)—C₁₋₃₀ alkyl-N(CH₃)C(O)—,
—C(O)NH—, —C(O)N(CH₃)—
—C(O)NH—C₁₋₃₀ alkyl-, —C(O)N(CH₃)—C₁₋₃₀ alkyl
—C(O)NH—C₁₋₃₀ alkyl-C(O)—, —C(O)N(CH₃)—C₁₋₃₀ alkyl-C(O)—,
—C(O)NH—C₁₋₃₀ alkyl-C(O)NH—, —C(O)N(CH₃)—C₁₋₃₀ alkyl-C(O)NH—,
—C(O)NH—C₁₋₃₀ alkyl-C(O)N(CH₃)—, —C(O)N(CH₃)—C₁₋₃₀ alkyl-C(O)N(CH₃)—,
—C(O)NH—C₁₋₃₀ alkyl-NH—, —C(O)N(CH₃)—C₁₋₃₀ alkyl-NH—,
—C(O)NH—C₁₋₃₀ alkyl-NHC(O)—, —C(O)N(CH₃)—C₁₋₃₀ alkyl-NHC(O)—,
—C(O)NH—C₁₋₃₀ alkyl-N(CH₃)—, —C(O)N(CH₃)—C₁₋₃₀ alkyl-N(CH₃)—,
—C(O)NH—C₁₋₃₀ alkyl-N(CH₃)C(O)—, —C(O)N(CH₃)—C₁₋₃₀ alkyl-N(CH₃)C(O)—,
—NH—, —NHC(O)—,
—NH—C₁₋₃₀ alkyl-, —NHC(O)—C₁₋₃₀ alkyl-,
—NH—C₁₋₃₀ alkyl-C(O)—, —NHC(O)—C₁₋₃₀ alkyl-C(O)—,
—NH—C₁₋₃₀ alkyl-C(O)NH—, —NHC(O)—C₁₋₃₀ alkyl-C(O)NH—,
—NH—C₁₋₃₀ alkyl-C(O)N(CH₃)—, —NHC(O)—C₁₋₃₀ alkyl-C(O)N(CH₃)—,
—NH—C₁₋₃₀ alkyl-NH—, —NHC(O)—C₁₋₃₀ alkyl-NH—,
—NH—C₁₋₃₀ alkyl-NHC(O)—, —NHC(O)—C₁₋₃₀ alkyl-NHC(O)—,
—NH—C₁₋₃₀ alkyl-N(CH₃)—, —NHC(O)—C₁₋₃₀ alkyl-N(CH₃)—,
—NH—C₁₋₃₀ alkyl-N(CH₃)C(O)—, —NHC(O)—C₁₋₃₀ alkyl-N(CH₃C(O)—,
—N(CH₃)—, —N(CH₃)C(O)—,
—N(CH₃)—C₁₋₃₀ alkyl-, —N(CH₃)C(O)—C₁₋₃₀ alkyl-,
—N(CH₃)—C₁₋₃₀ alkyl-C(O)—, —N(CH₃)C(O)—C₁₋₃₀ alkyl-C(O)—,
—N(CH₃)—C₁₋₃₀ alkyl-C(O)NH—, —N(CH₃)C(O)—C₁₋₃₀ alkyl-C(O)NH—,
—N(CH₃)—C₁₋₃₀ alkyl-C(O)N(CH₃)—, —N(CH₃)C(O)—C₁₋₃₀ alkyl-C(O)N(CH₃)—,
—N(CH₃)—C₁₋₃₀ alkyl-NH—, —N(CH₃)C(O)—C₁₋₃₀ alkyl-NH—,
—N(CH₃)—C₁₋₃₀ alkyl-NHC(O)—, —N(CH₃)C(O)—C₁₋₃₀ alkyl-NHC(O)—,
—N(CH₃)—C₁₋₃₀ alkyl-N(CH₃)—, —N(CH₃)C(O)—C₁₋₃₀ alkyl-N(CH₃)—,
—N(CH₃)—C₁₋₃₀ alkyl-N(CH₃)C(O)—, and —N(CH₃)C(O)—C₁₋₃₀ alkyl-N(CH₃C(O)—.

In some embodiments, L¹, L², L³, L⁴, L⁵, L⁶, and/or L is a compound linker comprising two or more (e.g., one, two, three, or four) simple spacer elements defined above. In some embodiments, the two or more elements of the compound linker are conjoined by an interrupting group (e.g, a sulfamide group (e.g., —NHS(O)₂NH—, —NHS(O)₂NHC(O)—, or —NHS(O)₂NHC(O)O—), or a heteroaryl or heteroaralkyl (e.g., a substituted triazole, a substituted DBCO, or a combination or derivative thereof). In some embodiments, the compound linker further comprises one or more units selected from the group consisting of:

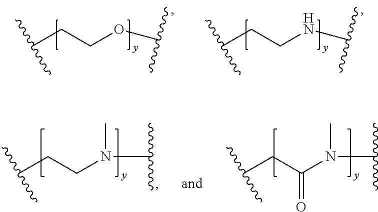

(e.g., wherein y is 1 to 20).

In some embodiments, L¹, L², L³, L⁴, L⁵, L⁶ and/or L⁷ is a combination of two or three simple spacers. In some embodiments, L¹, L², L³, L⁴, L⁵, L⁶ and/or L⁷ is a combination of two or three simple spacers, interrupted by (e.g., conjoined by) an amino acid, a dipeptide, or a tripeptide. In some embodiments, L¹, L², L³, L⁴, L⁵, L⁶ and/or L⁷ is a combination of two or three simple spacers, interrupted by (e.g., conjoined by) a cycloalkyl, heterocycloalkyl, aryl, or heteroaryl. In some embodiments, the interrupting group is a substituted triazole. In some embodiments, the interrupting group is an amino acid. In some embodiments, the interrupting group is: —S—, —S(O)—, —S(O)₂—, —NHS(O)₂NH—, —NHS(O)₂NHC(O)—, —NHS(O)₂NHC(O)O—,

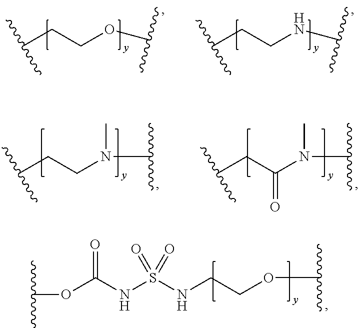

(wherein y is 1-20),

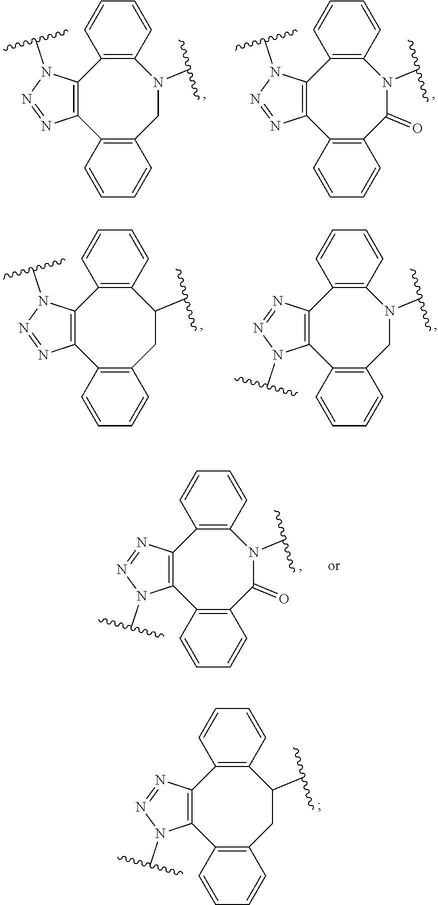

In some embodiments, L¹, L², L³, L⁴, L⁵, L⁶, and/or L¹ is a substituted or unsubstituted C₂₋₂₀ alkyl chain that is optionally interrupted one or more times by groups selected from —C(O)—, —C(O)NH—, —C(O)N(CH₃)—, —C(O)O—, —NH—, —N(CH₃)—, —NHC(O)—, —N(CH₃)C(O)—, —NHC(O)NH—, —O—, —S—, —S(O)—, —S(O)₂—, carbocyclyl, heterocyclyl, aralkyl, heteroaralkyl, or any combination thereof. In some embodiments, L¹, L², L³, L⁴, L⁵, L⁶, and/or L⁷ is a linker disclosed in WO2016207089, which is incorporated by reference in its entirety. In some embodiments, a linker comprises two simple spacers which are conjoined via an aryl or heteroaryl group (e.g., a triazole, a dibenzocyclooctyne, or a derivative thereof). For example, a linker may comprise a dibenzylcyclooctyne (DBCO) derivative such as a DBCO NHS ester, or a chemical group formed therefrom. For example, an interrupting group may include:

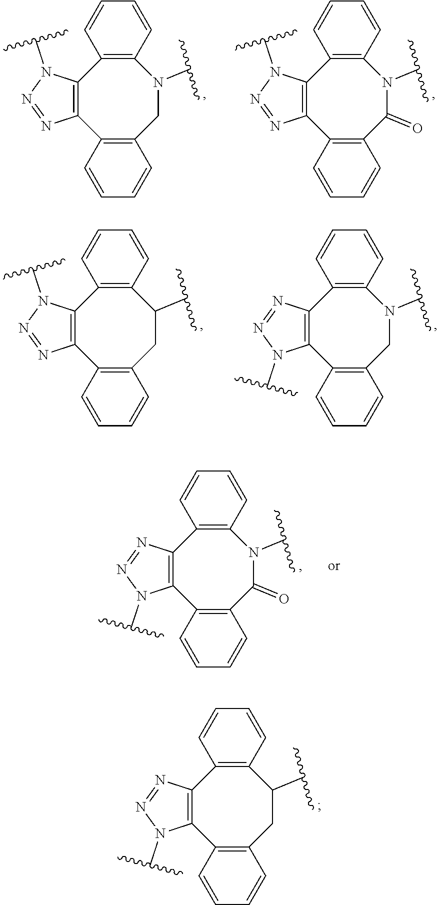

or a derivative thereof, wherein the interrupting group is substituted on each end to form a linker (e.g., L¹, L², L³, L⁴, L⁵, L⁶, and/or L⁷).

In some embodiments, L¹, L², L³, L⁴, L⁵, and/or L⁶ is a polymeric spacer having a structure represented by formula (i), formula (ii), or formula (iii) below:

—(CO)ₘ(CH₂)ₙ(OC₂₋₆ alkyl)ₒ(NH)ₚ(CO)_{q}—; (i)

—(CO)ᵣ(CH₂)ₛ(NR¹⁰C₁₋₆ alkyl)ₜ(NR¹¹)ᵤ(CO)ᵥ—; or (ii)

—(CO)ᵣ—(CH₂)ₛ—(NR¹⁰C(O)C₁₋₆ alkyl)ₜ-(NR¹¹)ᵤ—(CO)ᵥ—; (iii)
wherein:
R¹⁰ is, in each instance, independently selected from hydrogen or C₁₋₃ alkyl;
R¹¹ is, in each instance, independently selected from hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1;
v is 0 or 1.

In some embodiments, L¹, L², L³, L⁵, and/or L⁶ is a polymeric spacer selected from:
—C(O)—C₁₋₆ alkyl-[O—C₂₋₆ alkyl]₁₋₈-NHC(O)—,
—C(O)—C₁₋₆ alkyl-[O—C₂₋₆ alkyl]₁₋₈-N(CH₃)C(O)—,
—C(O)—C₁₋₆ alkyl-[NH—C₂₋₆ alkyl]₁₋₈-NHC(O)—,
—C(O)—C₁₋₆ alkyl-[NH—C₂₋₆ alkyl]₁₋₈-N(CH₃)C(O)—,
—C(O)—C₁₋₆ alkyl-[NHC(O)—C₁₋₆ alkyl]₁₋₈-NHC(O)—,
—C(O)—C₁₋₆ alkyl-[NHC(O)—C₁₋₆ alkyl]₁₋₈-N(CH₃)C(O)—,
—C(O)—C₁₋₆ alkyl-[N(CH₃)—C₂₋₆ alkyl]₁₋₈-NHC(O)—,
—C(O)—C₁₋₆ alkyl-[N(CH₃)—C₂₋₆ alkyl]₁₋₈-N(CH₃)C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈—N(CH₃)C(O)—,
—C(O)—C₁₋₆ alkyl-[O—C₂₋₆ alkyl]₁₋₈-NH—,
—C(O)—C₁₋₆ alkyl-[O—C₂₋₆ alkyl]₁₋₈-N(CH₃)—,
—C(O)—C₁₋₆ alkyl-[NH—C₂₋₆ alkyl]₁₋₈-NH—,
—C(O)—C₁₋₆ alkyl-[NH—C₂₋₆ alkyl]₁₋₈-N(CH₃)—,
—C(O)—C₁₋₆ alkyl-[NHC(O)—C₁₋₆ alkyl]₁₋₈-NH—,
—C(O)—C₁₋₆ alkyl-[NHC(O)—C₁₋₆ alkyl]₁₋₈-N(CH₃)—,
—C(O)—C₁₋₆ alkyl-[N(CH₃)—C₂₋₆ alkyl]₁₋₈-NH—,
—C(O)—C₁₋₆ alkyl-[N(CH₃)—C₂₋₆ alkyl]₁₋₈-N(CH₃)—,
—C(O)—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]t-—N(CH₃)—,
—C₁₋₆ alkyl-[O—C₂₋₆ alkyl]₁₋₈-NHC(O)—,
—C₁₋₆ alkyl-[O—C₂₋₆ alkyl]₁₋₈-N(CH₃)C(O)—,
—C₁₋₆ alkyl-[NH—C₂₋₆ alkyl]₁₋₈-NHC(O)—,
—C₁₋₆ alkyl-[NH—C₂₋₆ alkyl]₁₋₈-N(CH₃)C(O)—,
—C₁₋₆ alkyl-[N(CH₃)—C₂₋₆ alkyl]₁₋₈-NHC(O)—,
—C₁₋₆ alkyl-[N(CH₃)—C₂₋₆ alkyl]₁₋₈-N(CH₃)C(O)—,
—C₁₋₆ alkyl-[O—C₂₋₆ alkyl]₁₋₈-NH—,
—C₁₋₆ alkyl-[O—C₂₋₆ alkyl]₁₋₈-N(CH₃)—,
—C₁₋₆ alkyl-[NH—C₂₋₆ alkyl]₁₋₈-NH—,
—C₁₋₆ alkyl-[NH—C₂₋₆ alkyl]₁₋₈-N(CH₃)—,
—C₁₋₆ alkyl-[N(CH₃)—C₂₋₆ alkyl]₁₋₈-NH—, and
—C₁₋₆ alkyl-[N(CH₃)—C₂₋₆ alkyl]₁₋₈-N(CH₃)—.

In some embodiments, L⁴ is a polymeric amine or amide spacer (i.e., a polyamine or polyamide linker) selected from:
—C(O)—C₁₋₆ alkyl-[NH—C₂₋₆ alkyl]₁₋₈-NHC(O)—,
—C(O)—C₁₋₆ alkyl-[NH—C₂₋₆ alkyl]₁₋₈-N(CH₃)C(O)—,
—C(O)—C₁₋₆ alkyl-[NHC(O)—C₁₋₆ alkyl]₁₋₈-NHC(O)—,
—C(O)—C₁₋₆alkyl-[NHC(O)—C₁₋₆ alkyl]₁₋₈-N(CH₃)C(O)—,
—C(O)—C₁₋₆ alkyl-[N(CH₃)—C₂₋₆ alkyl]₁₋₈-NHC(O)—,
—C(O)—C₁₋₆ alkyl-[N(CH₃)—C₂₋₆ alkyl]₁₋₈-N(CH₃)C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈-N(CH₃)C(O)—,
—C(O)—C₁₋₆ alkyl-[NH—C₂₋₆ alkyl]₁₋₈-NH—,
—C(O)—C₁₋₆ alkyl-[NH—C₂₋₆ alkyl]₁₋₈-N(CH₃)—,
—C(O)—C₁₋₆ alkyl-[NHC(O)—C₁₋₆ alkyl]₁₋₈-NH—,
—C(O)—C₁₋₆ alkyl-[NHC(O)—C₁₋₆ alkyl]₁₋₈-N(CH₃)—,
—C(O)—C₁₋₆ alkyl-[N(CH₃)—C₂₋₆ alkyl]₁₋₈-NH—,
—C(O)—C₁₋₆ alkyl-[N(CH₃)—C₂₋₆ alkyl]₁₋₈-N(CH₃)—,
—C(O)—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]₁₋₈-N(CH₃)—,
—C₁₋₆ alkyl-[NH—C₂₋₆ alkyl]₁₋₈-NHC(O)—,
—C₁₋₆ alkyl-[NH—C₂₋₆ alkyl]₁₋₈-N(CH₃)C(O)—,
—C₁₋₆ alkyl-[N(CH₃)—C₂₋₆ alkyl]₁₋₈-NHC(O)—,
—C₁₋₆ alkyl-[N(CH₃)—C₂₋₆ alkyl]₁₋₈-N(CH₃)C(O)—,
—C₁₋₆ alkyl-[NH—C₂₋₆ alkyl]₁₋₈-NH—,
—C₁₋₆ alkyl-[NH—C₂₋₆ alkyl]₁₋₈-N(CH₃)—,
—C₁₋₆ alkyl-[N(CH₃)—C₂₋₆ alkyl]₁₋₈-NH—, and
—C₁₋₆ alkyl-[N(CH₃)—C₂₋₆ alkyl]₁₋₈-N(CH₃)—.

In some embodiments, L⁵ is a polymeric ether spacer (e.g., a PEG linker) selected from:
—C(O)—C₂₋₆ alkyl-[O—C₂₋₆ alkyl]₁₋₈-NHC(O)—,
—C(O)—C₂₋₆ alkyl-[O—C₂₋₆ alkyl]₁₋₈-N(CH₃)C(O)—,
—C(O)—C₂₋₆ alkyl-[O—C₂₋₆ alkyl]₁₋₈-NH—,
—C(O)—C₂₋₆ alkyl-[O—C₂₋₆ alkyl]₁₋₈-N(CH₃)—,
—C₂₋₆ alkyl-[O—C₂₋₆ alkyl]₁₋₈-NH—,
—C₂₋₆ alkyl-[O—C₂₋₆ alkyl]₁₋₈-NHC(O)—,
—C₂₋₆ alkyl-[O—C₂₋₆ alkyl]₁₋₈-N(CH₃)—, and
—C₂₋₆ alkyl-[O—C₂₋₆ alkyl]₁₋₈-N(CH₃)C(O)—.

In some embodiments, L⁷ is substituted C₁₂₋₃₀ alkyl, or a substituted or unsubstituted heteroalkyl having 15 to 60 (preferably 18 to 36) non-hydrogen atoms (preferably selected from C, N, O, and S). In some embodiments, L⁷ is a heteroalkyl having 15 to 60 atoms. In some embodiments, L⁷ is a heteroalkyl group that is conjoins IN to the rest of the molecule through 15 to 60 covalent bonds. In some embodiments, L^{T} is a heteroalkyl group. In some embodiments, L⁷ is a substituted or unsubstituted C₁₋₃₀ alkyl, or substituted or unsubstituted heteroalkyl. In some embodiments, L⁷ is a substituted or unsubstituted polyamine or a substituted or unsubstituted polyamide linker. In some embodiments, L⁷ is a substituted or unsubstituted C₁₋₆₀ alkyl, or substituted or unsubstituted heteroalkyl comprising 4-12 heteroalkyl units, each independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, or —NHS(O)₂NHC(O)—. In some embodiments, L⁷ is a substituted or unsubstituted C₁₋₃₀ alkyl, or substituted or unsubstituted heteroalkyl containing 4-12 Z groups; wherein each Z group is independently —(O—C₂₋₆ alkyl)-, —(NH—C₁₋₆ alkyl)-, —(N(C₁₋₃ alkyl)-C₁₋₆ alkyl), or —(N(C₁₋₃ alkyl)C(O)—C₁₋₆ alkyl). In some embodiments, L is a substituted or unsubstituted heteroalkyl containing 6 to 12 Z groups. In some embodiments, L⁷ is a substituted or unsubstituted heteroalkyl containing 6 to 10 Z groups. In some embodiments, each Z group is independently selected from —(OCH₂CH₂)—, —(OCH₂CH₂CH₂)—, —(OCH₂CH₂CH₂CH₂)—, —(OCH(CH₃)CH₂)—, —(OCH₂CH(CH₃))—, —(OC(CH₃)₂CH₂)—, —(OCH₂C(CH₃)₂)—, —(NHCH₂CH₂)—, —(NHCH₂CH₂CH₂)—, —(NHCH₂CH₂CH₂CH₂)—, —(NHCH(CH₃)CH₂)—, —(NHCH₂CH(CH₃))—, —(NHC(CH₃)₂CH₂)—, —(NHCH₂C(CH₃)₂)—, —(NHC(O)CH₂)—, —(NHC(O)CH₂CH₂)—, —(NHC(O)CHCH₃)—, —(NHC(O)C(CH₃)₂)—, —(NHC(O)C(CH₃)₂CH₂)—, —(N(CH₃)CH₂CH₂)—, —(N(CH₃)CH₂CH₂CH₂)—, —(N(CH₃)CH₂CH₂CH₂CH₂)—, —(N(CH₃)CH(CH₃)CH₂)—, —(N(CH₃)CH₂CH(CH₃))—, —(N(CH₃)C(CH₃)₂CH₂)—, —(N(CH₃)C(O)CH₂)—, —(N(CH₃)C(O)CH₂CH₂)—, —(N(CH₃)C(O)CHCH₃)—, —(N(CH₃)C(O)C(CH₃)₂)—, —(N(CH₃)C(O)C(CH₃)₂CH₂)—, and —(N(CH₃)CH₂C(CH₃)₂)—. In some embodiments, each Z group is independently selected from —(OCH₂CH₂)—, —(NHCH₂CH₂)—, —(N(CH₃)CH₂CH₂)—, and —(N(CH₃)C(O)CH₂)—. In some embodiments, Z groups are separated by an alkyl group, an amide, an alkylamide, an alkylamine, a polyaminoalkyl group, or an amino acid (including modified or synthetic amino acids). In some embodiments, L⁷ is a polyamine or polyamide linker. In some embodiments, L⁷ is a polyamine or polyamide linker comprising 6 to 12 units (Z) selected from: —(OCH₂CH₂)—, —(NHCH₂CH₂)—, —(N(CH₃)CH₂CH₂)—, and —(N(CH₃)C(O)CH₂)—.

In some embodiments, a spacer (e.g., L⁷) is an elongated linker having a structure represented by formula (i) or (ii) below:

—(CO)ₐ(C₀₋₆ alkyl)(Z¹C₁₋₆ alkyl)_{b}-Z²—(C₀₋₆ alkyl)(Z³C₁₋₆ alkyl)_{c}(Z⁴)_{d}(CO)ₑ—; or (i)

—(CO)ₐ—(C₀₋₆ alkyl)-(Z¹C(O)C₁₋₆ alkyl)_{b}-Z²—; (ii)
wherein:
Z¹ is in each occurrence independently —O—, —NH—, or —N(C₁₋₃ alkyl)-;
Z² is a bond, —C(O)—, —C(O)NH—, —C(O)—(C₁₋₆ alkyl)-C(O)—, —C(O)—(C₁₋₆ alkyl)-C(O)NH—, —C(O)—(C₁₋₆ alkyl)-C(O)N(CH₃)—, —C(O)NH—(C₁₋₆ alkyl)-C(O)NH—, —NH—, —N(C₁₋₆ alkyl)-, —N(C₁₋₆ alkyl)C(O)—, —NHC(O)—, —NHC(O)NH—, or —NHC(O)—(C₁₋₆ alkyl)-NHC(O)—;
Z³ is in each occurrence independently —O—, —NH—, or —N(C₁₋₃ alkyl)-;
Z⁴ is —O—, —NH—, or —N(C₁₋₃ alkyl)-;
a is 0 or 1;
b is 1 to 10;
c is 1 to 10;
d is 0 or 1; and
e is 0 or 1.

In some embodiments, a spacer (e.g., L⁷) is an elongated linker having a structure represented by formula (i) below:

In some embodiments, a spacer SP (e.g., L⁷) is an elongated spacer selected from the group consisting of:
—C(O)—C₂₋₄ alkyl-[O—C₂₋₄ alkyl]₂₋₄-NHC(O)—C₂₋₄ alkyl-[O—C₂₋₄ alkyl]₂₋₄-NHC(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-NHC(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-NHC(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-NHC(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C alkyl-[N(CH₃)—C₂₋₄ alkyl]₂₋₄-NHC(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NHC(O)—,
—C(O)—C₂₋₄ alkyl-[O—C₂₋₄ alkyl]₂₋₄-NHC(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-N(CH₃)C(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄-NHC(O)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄-N(CH₃)C(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—,
—C₂₋₄ alkyl-[O—C₂₋₄ alkyl]₂₋₄-NHC(O)—C₂₋₄ alkyl-[O—C₂₋₄ alkyl]₂₋₄-NHC(O)—,
—C₂₋₄ alkyl-[O—C₂₋₄ alkyl]₂₋₄-N(CH₃)C(O)—C₂₋₄ alkyl-[O—C₂₋₄ alkyl]₂₋₄-NHC(O)—, —C₁₋₄ alkyl-[NH—C₂₋₄ alkyl]₂₋₄-NHC(O)—C₁₋₄ alkyl-[NH—C₂₋₄ alkyl]₂₋₄-NHC(O)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-NHC(O)—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NHC(O)—,
—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NHC(O)—,
—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NHC(O)—,
—C₂₋₄ alkyl-[O—C₂₋₄ alkyl]₂₋₄-NHC(O)—C₂₋₄ alkyl-[O—C₂₋₄ alkyl]₂₋₄-N(CH₃)C(O)—,
—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C₁₋₄ alkyl-[NH—C₂₋₄ alkyl]₂₋₄-NHC(O)—C₁₋₄ alkyl-[NH—C₂₋₄ alkyl]₂₋₄-N(CH₃)C(O)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)C(O)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)C(O)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)C(O)—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄-NHC(O)—C₂₋₄ alkyl-[O—C₂₋₄ alkyl]₂₋₄-NH—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-NH—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-NH—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-NH—,
—C(O)—C₁₋₄ alkyl-[N(CH₃)—C₂₋₄ alkyl]₂₋₄-NH C(O)—C alkyl-[N(CH₃)—C₂₋₄ alkyl]₂₋₄-NH—,
—C(O)—C₁₋₄ alkyl-[N(CH₃)—C₂₋₄ alkyl]₂₋₄-N(CH₃)C(O)—C alkyl-[N(CH₃)—C₂₋₄ alkyl]₂₋₄-NH—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NH—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NH—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NH—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NH—,
—C(O)—C₂₋₄ alkyl-[O—C₂₋₄ alkyl]₂₋₄-NHC(O)—C₂₋₄ alkyl-[O—C₂₋₄ alkyl]₂₋₄-N(CH₃)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-N(CH₃)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)—,
—C(O)—C₂₋₄ alkyl-[O—C₂₋₄ alkyl]₂₋₄-N(CH₃)C(O)—C alkyl-[N(CH₃)—C₂₋₄ alkyl]₂₋₄-N(CH₃)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)—,
—C₂₋₄ alkyl-[O—C₂₋₄ alkyl]₂₋₄-NHC(O)—C₂₋₄ alkyl-[O—C₂₋₄ alkyl]₂₋₄-NH—,
—C₂₋₄ alkyl-[O—C₂₋₄ alkyl]₂₋₄-N(CH₃)C(O)—C₂₋₄ alkyl-[O—C₂₋₄ alkyl]₂₋₄-NH—,
—C₁₋₄ alkyl-[NH—C₂₋₄ alkyl]₂₋₄-NHC(O)—C₁₋₄ alkyl-[NH—C₂₋₄ alkyl]₂₋₄-NH—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-NH—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NH—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NH—,
—C₂₋₄ alkyl-[O—C₂₋₄ alkyl]₂₋₄-NHC(O)—C₁₋₄ alkyl-[N(CH₃)—C₂₋₄ alkyl]₂₋₄-NH—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NH—,
—C₁₋₄ alkyl-[N(CH₃)—C₂₋₄ alkyl]₂₋₄-NHC(O)—C₂₋₄ alkyl-[O—C₂₋₄ alkyl]₂₋₄-NH—,
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NH—,
—C₂₋₄ alkyl-[O—C₂₋₄ alkyl]₂₋₄-NHC(O)—C₂₋₄ alkyl-[O—C₂₋₄ alkyl]₂₋₄-N(CH₃)—,
—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)—,
—C₁₋₄ alkyl-[NH—C₂₋₄ alkyl]₂₋₄-NHC(O)—C₁₋₄ alkyl-[NH—C₂₋₄ alkyl]₂₋₄-N(CH₃)—,
—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)—,
—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)—,
—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)—,
—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)—,
—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)—,
—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)—, and
—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)—.

In some embodiments, a spacer (e.g., L¹, L², L³, L⁴, L⁵, L⁶, or L⁷) further comprises a spanner, wherein the spanner is a linker that conjoins the spacer to an adjacent group (e.g., EL, A¹, IN, MOD). A spanner can be a substituted or unsubstituted alkyl, or a substituted or unsubstituted heteroalkyl linker, typically comprising about 1 to about 20 atoms selected from carbon, nitrogen, and oxygen. Preferably, a spanner is a short alkyl or heteroalkyl group having functional groups at its ends that are configured to form a stable bond (e.g., a peptide bond) with an adjacent group. Examples of spanners include groups such as:
#C(O)—C₂₋₆ alkyl-C(O)—, #C(O)NH—C₂₋₆ alkyl-C(O)—,
#C(O)N(CH₃)—C₂₋₆ alkyl-C(O)—, #NH—C₂₋₆ alkyl-C(O)—,
#NHC(O)—C₂₋₆ alkyl-C(O)—, #N(CH₃)—C₂₋₆ alkyl-C(O)—,
#N(CH₃)C(O)—C₂₋₆ alkyl-C(O)—, #C(O)—C₂₋₆ alkyl-C(O)NH—,
#C(O)NH—C₂₋₆ alkyl-C(O)NH—, #C(O)N(CH₃)—C₂₋₆ alkyl-C(O)NH—,
#NH—C₂₋₆ alkyl-C(O)NH—, #NHC(O)—C₂₋₆ alkyl-C(O)NH—,
#N(CH₃)—C₂₋₆ alkyl-C(O)NH—, #N(CH₃)C(O)—C₂₋₆ alkyl-C(O)NH—,
#C(O)—C₂₋₆ alkyl-C(O)N(CH₃)—, #C(O)NH—C₂₋₆ alkyl-C(O)N(CH₃)—,
#C(O)N(CH₃)—C₂₋₆ alkyl-C(O)N(CH₃)—, #NH—C₂₋₆ alkyl-C(O)N(CH₃)—,
#NHC(O)—C₂₋₆ alkyl-C(O)N(CH₃)—, #N(CH₃)—C₂₋₆ alkyl-C(O)N(CH₃)—,
#N(CH₃)C(O)—C₂₋₆ alkyl-C(O)N(CH₃)—, #C(O)—C₂₋₆ alkyl-NH—,
#C(O)NH—C₂₋₆ alkyl-NH—, #C(O)N(CH₃)—C₂₋₆ alkyl-NH—,
#NH—C₂₋₆ alkyl-NH—, #NHC(O)—C₂₋₆ alkyl-NH—,
#N(CH₃)—C₂₋₆ alkyl-NH—, #N(CH₃)C(O)—C₂₋₆ alkyl-NH—,
#C(O)—C₂₋₆ alkyl-NHC(O)—, #C(O)NH—C₂₋₆ alkyl-NHC(O)—,
#C(O)N(CH₃)—C₂₋₆ alkyl-NHC(O)—, #NH—C₂₋₆ alkyl-NHC(O)—,
#NHC(O)—C₂₋₆ alkyl-NHC(O)—, #N(CH₃)—C₂₋₆ alkyl-NHC(O)—,
#N(CH₃)C(O)—C₂₋₆ alkyl-NHC(O)—, #C(O)—C₂₋₆ alkyl-N(CH₃)—,
#C(O)NH—C₂₋₆ alkyl-N(CH₃)—, #C(O)N(CH₃)—C₂₋₆ alkyl-N(CH₃)—,
#NH—C₂₋₆ alkyl-N(CH₃)—, #NHC(O)—C₂₋₆ alkyl-N(CH₃)—,
#N(CH₃)—C₂₋₆ alkyl-N(CH₃)—, #N(CH₃)C(O)—C₂₋₆ alkyl-N(CH₃)—,
#C(O)—C₂₋₆ alkyl-N(CH₃)C(O)—, #C(O)NH—C₂₋₆ alkyl-N(CH₃)C(O)—,
#C(O)N(CH₃)—C₂₋₆ alkyl-N(CH₃)C(O)—, #NH—C₂₋₆ alkyl-N(CH₃)C(O)—,
#NHC(O)—C₂₋₆ alkyl-N(CH₃)C(O)—, #N(CH₃)—C₂₋₆ alkyl-N(CH₃)C(O)—,
#N(CH₃)C(O)—C₂₋₆ alkyl-N(CH₃)C(O)—, #C(O)—C₂₋₆ alkyl-,
#C(O)NH—C₂₋₆ alkyl-, #C(O)N(CH₃)—C₂₋₆ alkyl-,
#NH—C₂₋₆ alkyl-, #NHC(O)—C₂₋₆ alkyl-,
#N(CH₃)—C₂₋₆ alkyl-, #N(CH₃)C(O)—C₂₋₆ alkyl-;
#C₂₋₆ alkyl-C(O)—, #C₂₋₆ alkyl-C(O)NH—,
#C₂₋₆ alkyl-C(O)N(CH₃)—, #C₂₋₆ alkyl-NH—,
#C₂₋₆ alkyl-NHC(O)—, #C₂₋₆ alkyl-N(CH₃)—, or
#C₂₋₆ alkyl-N(CH₃)C(O)—,
wherein #denotes a bond to the spacer, and — denotes a bond to the adjacent group.

In some embodiments, a spanner is #C(O)—C₂₋₆ alkyl-C(O)— or #NH—C₂₋₆ alkyl-NH—. In some embodiments, a spanner is #C(O)(CH₂CH₂CH₂)C(O)— or #NH(CH₂CH₂)NH—. As used herein spanners are denoted S¹, S², S³, etc. corresponding to the linker to which they are attached. Alternatively, a spanner may be described as a member of A¹, in which case the S¹, S², S³, numbering still pertains to the linker to which A¹ is bonded. In some embodiments, a spanner is a bond. In some embodiments, a spanner is absent.

In some embodiments, L¹ is selected from:

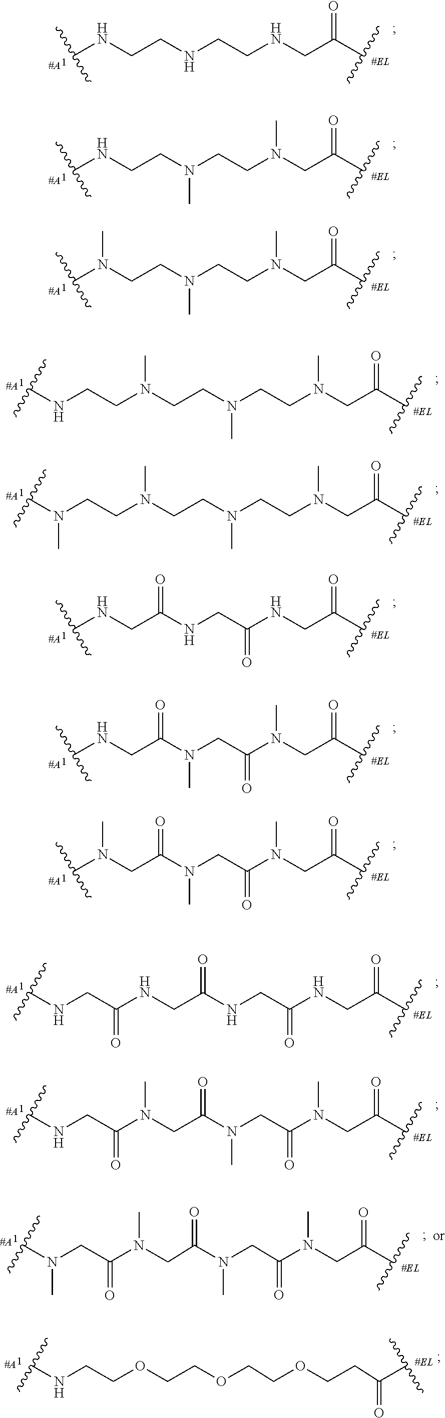

wherein #EL denotes a bond to EL (or SIL); and #A¹ denotes a bond to A¹.

In some embodiments, L² and L³ are each independently selected from:

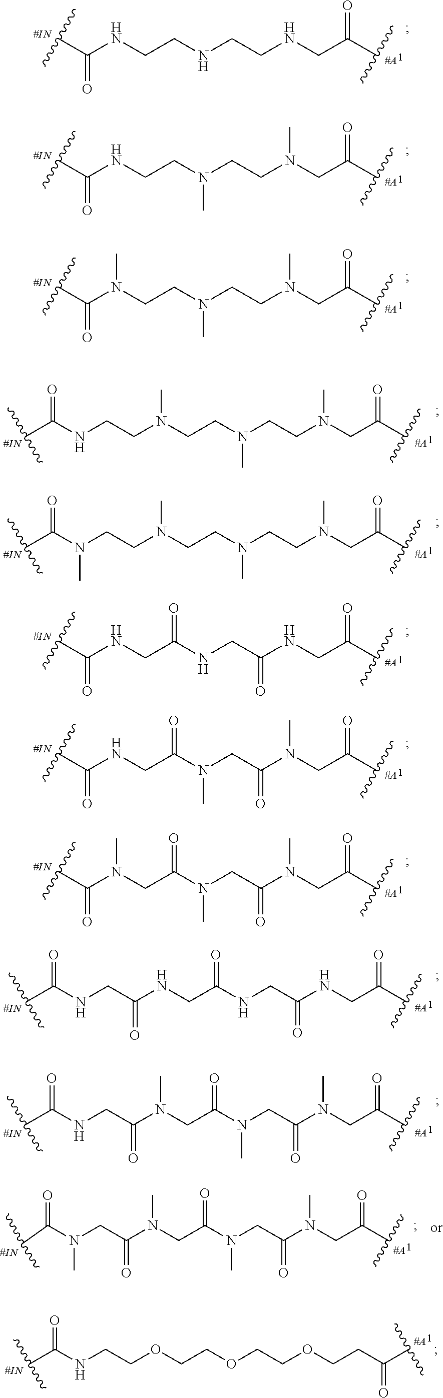

wherein #IN denotes a bond to IN or MOD; and #A¹ denotes a bond to A¹.

In some embodiments, L⁴ is:

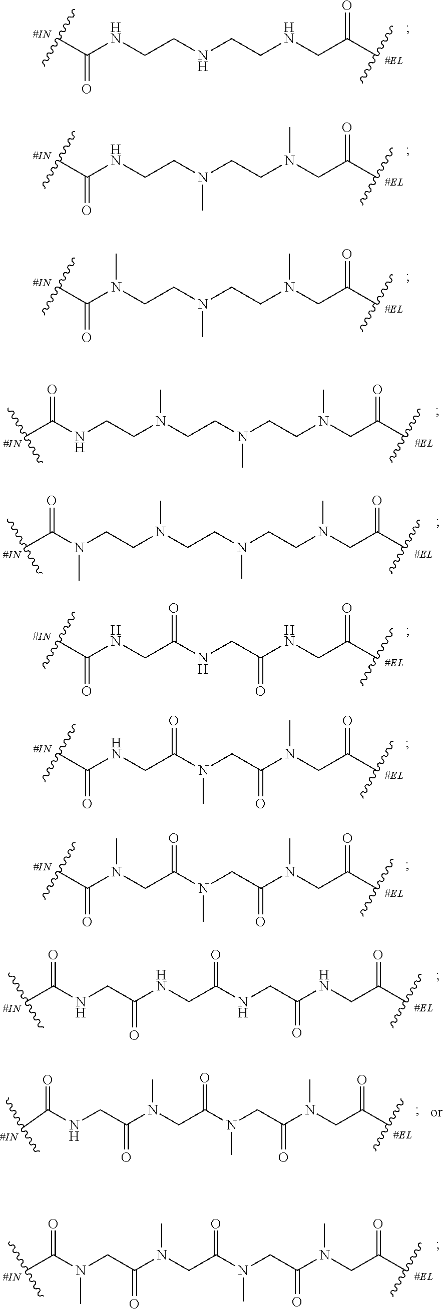

wherein #EL denotes a bond to EL (or SIL); and #IN denotes a bond to IN.

In some embodiments, L⁴ is:

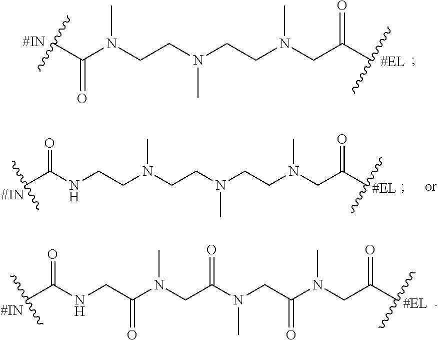

In some embodiments, L⁵ is:

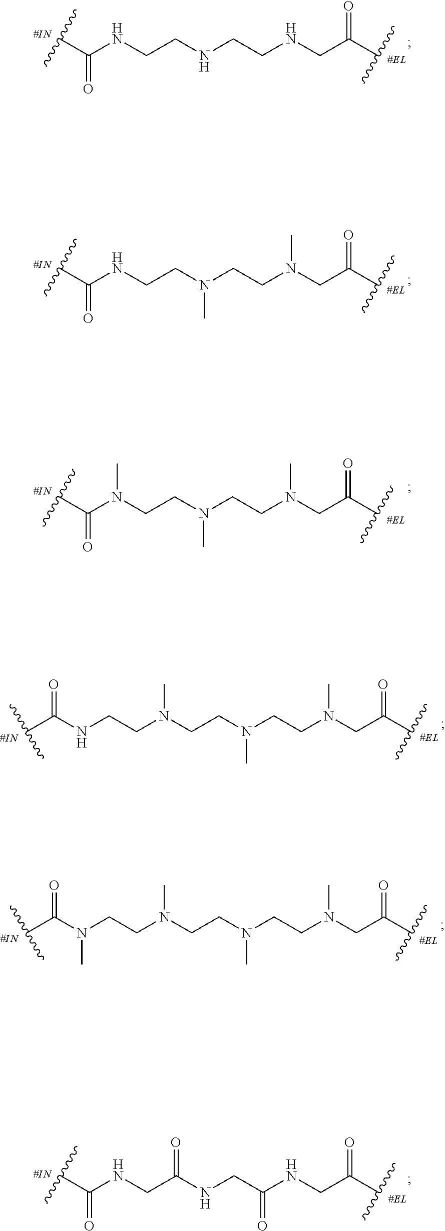 [SMILES: *C(=O)CNCCNCCNC(*)=O ; *C(=O)CN(C)CCN(C)CCNC(*)=O ; *C(=O)CN(C)CCN(C)CCN(C)C(*)=O ; *C(=O)CN(C)CCN(C)CCN(C)CCNC(*)=O ; *C(=O)CN(C)CCN(C)CCN(C)CCN(C)C(*)=O ; *C(=O)CNC(=O)CNC(=O)CNC(*)=O]

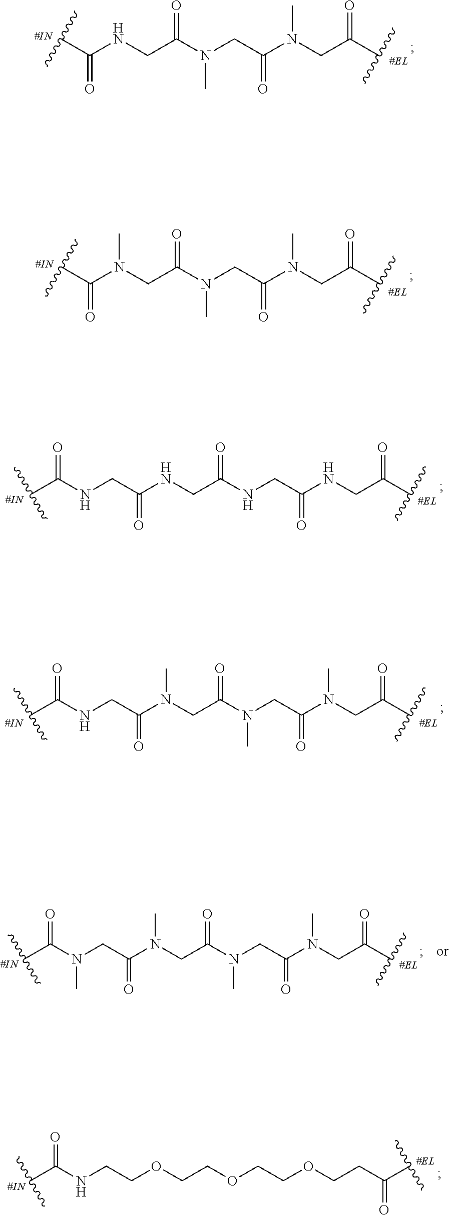 [SMILES: *C(=O)CN(C)C(=O)CN(C)C(=O)CNC(*)=O ; *C(=O)CN(C)C(=O)CN(C)C(=O)CN(C)C(*)=O ; *C(=O)CNC(=O)CNC(=O)CNC(=O)CNC(*)=O ; *C(=O)CN(C)C(=O)CN(C)C(=O)CN(C)C(=O)CNC(*)=O ; *C(=O)CN(C)C(=O)CN(C)C(=O)CN(C)C(=O)CN(C)C(*)=O ; *C(=O)CCOCCOCCOCCNC(*)=O]

wherein #EL denotes a bond to EL (or SIL); and #IN denotes a bond to IN.

In some embodiments, L⁵ is:

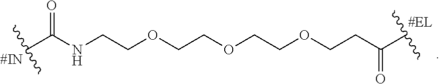 [SMILES: *C(=O)CCOCCOCCOCCNC(*)=O]

In some embodiments, L¹ is: —C₀₋₁₂ alkyl-C(O)—; —C₀₋₁₂ alkyl-C(O)NH—; —C₀₋₁₂ alkyl-C(O)N(CH₃)—; —C₀₋₁₂ alkyl-NH—; —C₀₋₁₂ alkyl-NHC(O)—; —C₀₋₁₂ alkyl-N(CH₃)—; or —C₀₋₁₂ alkyl-N(CH₃)C(O)—;

In some embodiments, L¹ is: —C(O)—.

In some embodiments, L is:

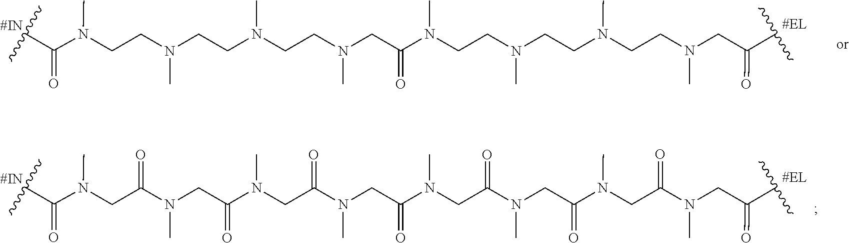 [SMILES: *C(=O)CN(C)CCN(C)CCN(C)CCN(C)C(=O)CN(C)CCN(C)CCN(C)CCN(C)C(*)=O ; *C(=O)CN(C)C(=O)CN(C)C(=O)CN(C)C(=O)CN(C)C(=O)CN(C)C(=O)CN(C)C(=O)CN(C)C(=O)CN(C)C(*)=O]

wherein #IN represents a bond to an integrin binder (IN), and #EL represents a bond to an enzymatically-cleavable linker (EL).

In some embodiments, a linker (L), e.g., L¹, L², L³, L⁴, L⁵, L⁶, L⁷, is (or comprises) a group denoted as L1 in WO2016207089, which is incorporated by reference in its entirety.

### Trivalent Radicals (A¹)

In some embodiments, a trivalent radical A¹ is a moiety containing 1 to 100 atoms selected from H, C, N, O, and S, configured to bond to linkers L¹, L², and L³. In some embodiments, A¹ contains a central atom or group (Y) that is N or CH. In some embodiments, the central atom or group (Y) has one or more arms (e.g., alkyl or heteroalkyl chains, optionally substituted with, or interrupted by, one or more groups independently selected from carbonyls (—C(O)—), ethers (—O—), amines (e.g., —NH— or —N(CH₃)—), or amides (e.g., —C(O)NH—, —C(O)N(CH₃)—, —NHC(O)—, or —N(CH₃)C(O)—, alternatively referred to as amide linkers). In some embodiments, A¹ is trivalent heteroalkyl radical. In some embodiments, A¹ is trivalent radical with amide linker arms (e.g., B¹, B², B³). In some embodiments, the trivalent radical is referred to as a branching unit, a branching group. In some embodiments, A of Formula (01) or Formula (05) is A¹. In some embodiments, A¹ is a trivalent heteroaryl, heteroaralkyl, aryl, aralkyl, heteroalkyl-aryl, heteroalkyl-heteroaryl radical. In some embodiments, A¹ contains a central atom or group (Y) that is a carbocycle (e.g., cycloalkyl or aryl) or heterocycle (e.g., heterocycloalkyl or heteroaryl). In some embodiments, (Y) is phenyl, triazinyl, or triazolyl, each of which is optionally substituted by 1-3 heteroalkyl arms. In some embodiments, a linker (e.g., L¹, L², or L³) is or comprises an alkyl, alkenyl, alkynyl, heteroalkyl, cycloalkyl, aryl, heterocyclyl, heteroaryl, aralkyl, heteroaralkyl, alkyl-heterocyclyl, heteroalkyl-heterocyclyl, alkyl-aralkyl, heteroalkyl-aralkyl, alkyl-heteroaralkyl, heteroalkyl-heteroaralkyl, alkyl-heterocycloalkyl, or any combination thereof. In some embodiments, a linker (e.g., L¹, L², or L³) is a heteroalkyl or alkyl linker, optionally interrupted with an aryl, heteroaryl, cycloalkyl, or heterocyclyl group. In some embodiments, the linker is interrupted with a click group (e.g., a substituted triazole).

In some embodiments, A or A¹ is a trivalent linker, optionally comprising arms or spanners, that is configured to conjoin a payload to two integrin binders, or to an integrin binder and a MOD group (e.g., via a spacer L and/or an enzymatic linker EL). In some embodiments, A or A¹ is a trivalent linker. In some embodiments, A or A¹ is a trivalent linker comprising arms B¹, B², and B³, and optionally further comprising spanners S¹, S², and S³, wherein the trivalent linker is configured to form an amide bond with an adjacent group (e.g., L¹, L², L³, IN, MOD, or EL). In some embodiments, A or A¹ is a trivalent amide linker. In this context, a trivalent amide linker can be a chemical group having three valencies (or capable of forming bonds to 3 agents), and generally composed of alkyl, carbonyl, and amine groups, and preferably capable of forming an amide bond with an adjacent group (e.g., L¹, L², L³, IN, MOD, or EL). In some embodiments, the trivalent radical is a trivalent amide linker. In some embodiments, the trivalent amide linker comprises a central amino acid (e.g., lysine, glutamine, glutamate, etc.), wherein the amino acid forms a bond with an adjacent group (e.g., L¹, L², L³′ IN, MOD, or EL), optionally bridged via a linking arm (e.g., B¹, B², B³) and/or a spanner (i.e., S¹, S², or S³). Preferably, a trivalent radical as described herein conjoins a payload (e.g., an enzymatically-cleavable payload (CP)) with two integrin binders, or with an integrin binder and a group MOD, and wherein the payload, integrin binder(s) and MOD may be conjoined via a linker/spacer (L¹, L², or L³).

In some embodiments, A¹ has a structure represented by the formula:

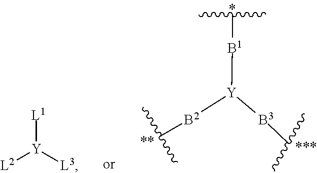

wherein (Y) is N, CH, phenyl, or heteroaryl, and each of B¹, B², and B³ represents an arm that connects to a corresponding spacer (e.g., L¹, L², and L³ respectively) and wherein * denotes a bond to L¹, ** denotes a bond to L², *** denotes a bond to L³.

In some embodiments, A¹ is a trivalent radical of the formula:

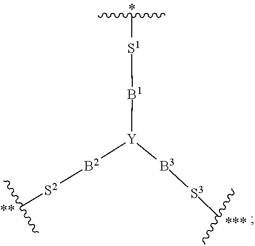

wherein:
* denotes a bond to L¹,
** denotes a bond to L²,
*** denotes a bond to L³,
each of S¹, S², S³, is spanner; and
each of B¹, B², B³, is a linking arm.
and Y is CH or N.

In some embodiments, one or more of S¹, S², and S³ is a bond. In some embodiments, one or more of S¹, S², and S³ is —C(O)—C₂₋₆ alkyl-C(O)—, —N(CH₃)—C₁₋₆ alkyl-N(CH₃)—, —NH—C₁₋₆ alkyl-NH—, or —NH—C₁₋₆ alkyl-N(CH₃)—. In some embodiments, S¹, S², and/or S³ is —C(O)—C₂₋₆ alkyl-C(O)— or —NH—C₁₋₆ alkyl-NH—.

In some embodiments, each B¹, B², and B³, is an amide linker (e.g., an alkyl amide and/or a polyamide linker). In some embodiments, each B¹, B², and B³, is an amide linker comprising 1 to about 32 atoms. In some embodiments, the 1 to 32 atoms are selected from carbon, nitrogen, and oxygen.

In some embodiments, each B¹, B², and B³, is selected from:
—C(O)—;
—C(O)NH—;
—C(O)N(CH₃)—;
—C(O)NH—C₀₋₆ alkyl-C(O)—;
—C(O)NH—C₁₋₆ alkyl-C(O)NH—;
—C(O)NH—C₁₋₆ alkyl-C(O)N(CH₃)—;
—C(O)NH—C₁₋₆ alkyl-NH—;
—C(O)NH—C₁₋₆ alkyl-N(CH₃)—;
—C(O)NH—C₁₋₆ alkyl-NHC(O)—;
—C(O)NH—C₁₋₆ alkyl-N(CH₃)C(O)—;
—C(O)N(CH₃)—C₁₋₆ alkyl-C(O)—;
—C(O)N(CH₃)—C₁₋₆ alkyl-C(O)NH—;
—C(O)N(CH₃)—C₁₋₆ alkyl-C(O)N(CH₃)—;
—C(O)NH—C₁₋₆ alkyl-NH—;
—C(O)NH—C₁₋₆ alkyl-N(CH₃)—;
—C(O)N(CH₃)—C₁₋₆ alkyl-NHC(O)—;
—C(O)N(CH₃)—C₁₋₆ alkyl-N(CH₃)C(O)—;
—C(O)—C₁₋₆ alkyl-C(O)—;
—C(O)—C₁₋₆ alkyl-C(O)NH—;
—C(O)—C₁₋₆ alkyl-C(O)N(CH₃)—;
—C(O)—C₁₋₆ alkyl-NH—;
—C(O)—C₁₋₆ alkyl-N(CH₃)—;
—C(O)—C₁₋₆ alkyl-NHC(O)—;
—C(O)—C₁₋₆ alkyl-N(CH₃)C(O)—;
—C₀₋₆ alkyl-C(O)—;
—C₀₋₆ alkyl-C(O)NH—;
—C₀₋₆ alkyl-C(O)N(CH₃)—;
—C₀₋₆ alkyl-C(O)NH—C₁₋₆ alkyl-C(O)—;
—C₀₋₆ alkyl-C(O)NH—C₁₋₆ alkyl-C(O)NH—;
—C₀₋₆ alkyl-C(O)NH—C₁₋₆ alkyl-C(O)N(CH₃)—;
—C₀₋₆ alkyl-C(O)NH—C₁₋₆ alkyl-NH—;
—C₀₋₆ alkyl-C(O)NH—C₁₋₆ alkyl-N(CH₃)—;
—C₀₋₆ alkyl-C(O)NH—C₁₋₆ alkyl-NHC(O)—;
—C₀₋₆ alkyl-C(O)NH—C₁₋₆ alkyl-N(CH₃)C(O)—;
—C₀₋₆ alkyl-C(O)N(CH₃)—C₁₋₆ alkyl-C(O)—;
—C₀₋₆ alkyl-C(O)N(CH₃)—C₁₋₆ alkyl-C(O)NH—;
—C₀₋₆ alkyl-C(O)N(CH₃)—C₁₋₆ alkyl-C(O)N(CH₃)—;
—C₀₋₆ alkyl-C(O)NH—C₁₋₆ alkyl-NH—;
—C₀₋₆ alkyl-C(O)NH—C₁₋₆ alkyl-N(CH₃)—;
—C₀₋₆ alkyl-C(O)N(CH₃)—C₁₋₆ alkyl-NHC(O)—;
—C₀₋₆ alkyl-C(O)N(CH₃)—C₁₋₆ alkyl-N(CH₃)C(O)—;
—C₀₋₆ alkyl-C(O)—C₁₋₆ alkyl-C(O)—;
—C₀₋₆ alkyl-C(O)—C₁₋₆ alkyl-C(O)NH—;
—C₀₋₆ alkyl-C(O)—C₁₋₆ alkyl-C(O)N(CH₃)—;
—C₀₋₆ alkyl-C(O)—C₁₋₆ alkyl-NH—;
—C₀₋₆ alkyl-C(O)—C₁₋₆ alkyl-N(CH₃)—;
—C₀₋₆ alkyl-C(O)—C₁₋₆ alkyl-NHC(O)—;
—C₀₋₆ alkyl-C(O)—C₁₋₆ alkyl-N(CH₃)C(O)—;
—C₀₋₆ alkyl-NH—;
—C₀₋₆ alkyl-NHC(O)—;
—C₀₋₆ alkyl-NHC(O)—C₁₋₆ alkyl-C(O)—;
—C₀₋₆ alkyl-NHC(O)—C₁₋₆ alkyl-C(O)NH—;
—C₀₋₆ alkyl-NHC(O)—C₁₋₆ alkyl-C(O)N(CH₃)—;
—C₀₋₆ alkyl-NHC(O)—C₁₋₆ alkyl-NH—;
—C₀₋₆ alkyl-NHC(O)—C₁₋₆ alkyl-N(CH₃)—;
—C₀₋₆ alkyl-NHC(O)—C₁₋₆ alkyl-NHC(O)—;
—C₀₋₆ alkyl-NHC(O)—C₁₋₆ alkyl-N(CH₃)C(O)—;
—C₀₋₆ alkyl-N(CH₃)—;
—C₀₋₆ alkyl-N(CH₃)C(O)—;
—C₀₋₆ alkyl-N(CH₃)C(O)—C₁₋₆ alkyl-C(O)—;
—C₀₋₆ alkyl-N(CH₃)C(O)—C₁₋₆ alkyl-C(O)NH—;
—C₀₋₆ alkyl-N(CH₃)C(O)—C₁₋₆ alkyl-C(O)N(CH₃)—;
—C₀₋₆ alkyl-N(CH₃)C(O)—C₁₋₆ alkyl-NH—;
—C₀₋₆ alkyl-N(CH₃)C(O)—C₁₋₆ alkyl-N(CH₃)—;
—C₀₋₆ alkyl-N(CH₃)C(O)—C₁₋₆ alkyl-NHC(O)—; and
—C₀₋₆ alkyl-N(CH₃)C(O)—C₁₋₆ alkyl-N(CH₃)C(O)—.

In some embodiments, A¹ is an amino acid or a derivative thereof. In some embodiments, A¹ is a Lys (e.g., L-Lys or D-Lys), Glu (L-Glu or D-Glu), or Asp (L-Asp or D-Asp), or a derivative thereof (e.g., substituted with one or more arms (e.g., B¹, B², or B³)). In some embodiments, A¹ has one of the following structures:
*C(O)—C₀₋₆ alkyl-Y(C₀₋₆ alkyl-NH**)(C₀₋₆ alkyl-NH***),
*C(O)—C₀₋₆ alkyl-Y(C₀₋₆ alkyl-NH**)(C₀₋₆ alkyl-C(O)***),
*C(O)—C₀₋₆ alkyl-Y(C₀₋₆ alkyl-C(O)**)(C₀₋₆ alkyl-C(O)***),
*C(O)—C₀₋₆ alkyl-C(O)NH—Y(C₀₋₆ alkyl-NH**)(C₀₋₆ alkyl-NH***),
*C(O)—C₀₋₆ alkyl-C(O)NH—Y(C₀₋₆ alkyl-NH**)(C₀₋₆ alkyl-C(O)***),
*C(O)—C₀₋₆ alkyl-C(O)NH—Y(C₀₋₆ alkyl-C(O)**)(C₀₋₆ alkyl-C(O)***),
*C(O)—C₀₋₆ alkyl-C(O)NH—Y(C₀₋₆ alkyl-C(O)NH—C₀₋₆ alkyl-NH**)(C₀₋₆ alkyl-C(O)NH—C₀₋₆ alkyl-NH**);
wherein each of ***, and *** denotes a bond to a spacer L¹, L², or L³; and Y is N or CH. In some embodiments, Y is lysine or glutamine.

In some embodiments, A¹ has one of the following structures:

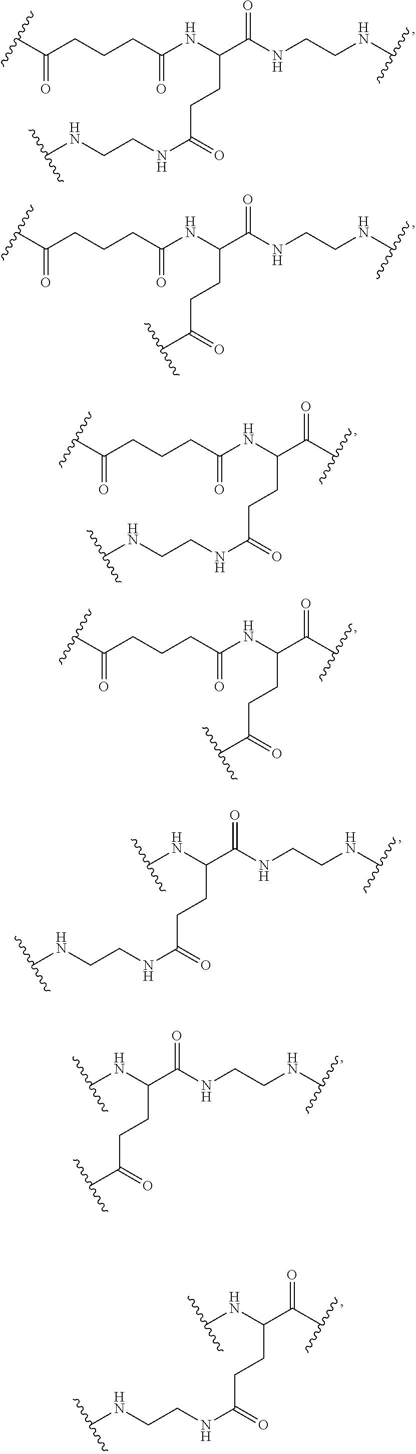

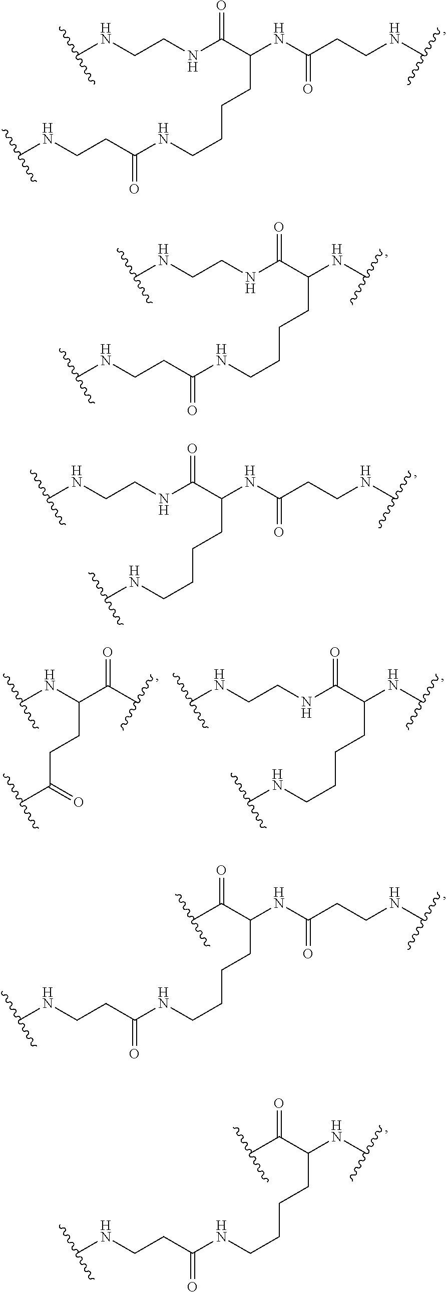

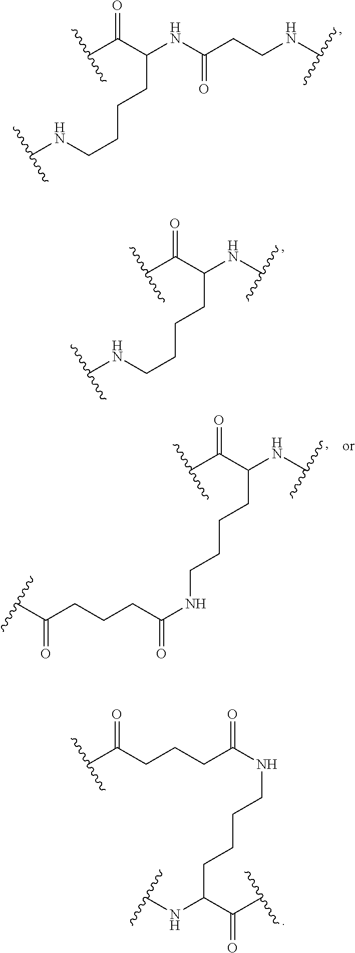 [SMILES: *NCCCCC(NC(=O)CCN*)C(*)=O ; *NCCCCC(N*)C(*)=O ; *NC(CCCCNC(=O)CCCC(*)=O)C(*)=O ; *NC(CCCCNC(=O)CCCC(*)=O)C(*)=O]

In some embodiments, A¹ is of Formula (A¹-1a):

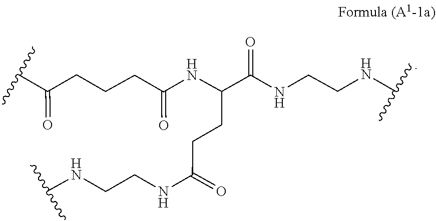 [SMILES: *NCCNC(=O)CCC(NC(=O)CCCC(*)=O)C(=O)NCCN*]

In some embodiments, A¹ is of Formula (A¹-1b):

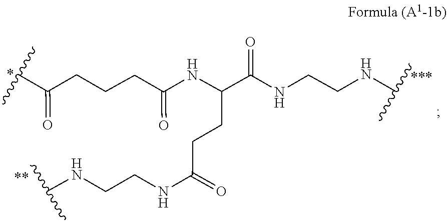 [SMILES: *NCCNC(=O)CCC(NC(=O)CCCC(*)=O)C(=O)NCCN*]

wherein * denotes a bond to L¹, ** denotes a bond to L¹, *** denotes a bond to L³.

In some embodiments, A¹ is of Formula (A¹-1c) or Formula (A¹-1d):

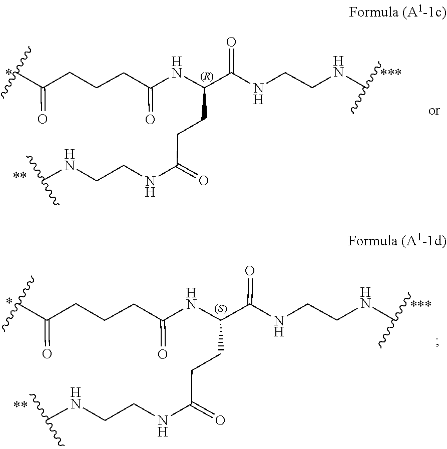

wherein * denotes a bond to L¹, ** denotes a bond to L², *** denotes a bond to L³.

In some embodiments, A¹ is of Formula (A¹-2a):

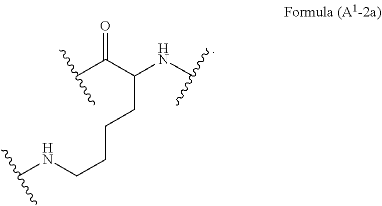 [SMILES: *NCCCCC(N*)C(*)=O]

In some embodiments, A¹ is of Formula (A¹-2b):

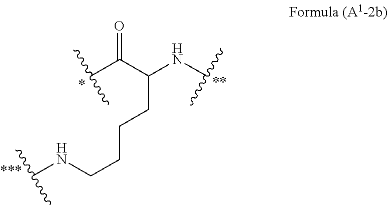 [SMILES: *NCCCCC(N*)C(*)=O]

wherein * denotes a bond to L¹, ** denotes a bond to L², *** denotes a bond to L³.

In some embodiments, A¹ is of Formula (A¹-2c) or Formula (A¹-2d):

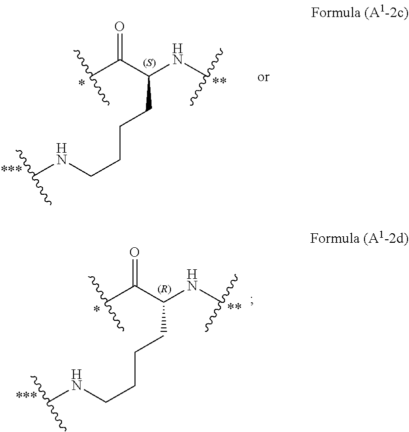

wherein * denotes a bond to L¹, ** denotes a bond to L², *** denotes a bond to L³.

In some embodiments, A¹ is of Formula (A¹-3a):

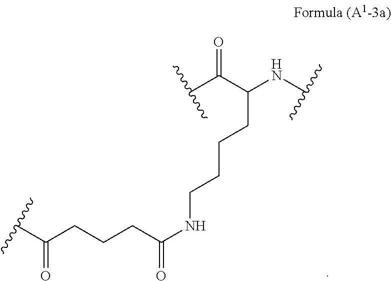 [SMILES: *NC(CCCCNC(=O)CCCC(*)=O)C(*)=O]

In some embodiments, A¹ is of Formula (A¹-3b):

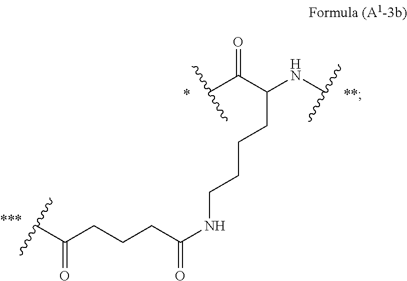 [SMILES: *NC(CCCCNC(=O)CCCC(*)=O)C(*)=O]

wherein * denotes a bond to L¹, ** denotes a bond to L², *** denotes a bond to L³.

In some embodiments, A¹ is of Formula (Ae-3c) or Formula (A¹-3d):

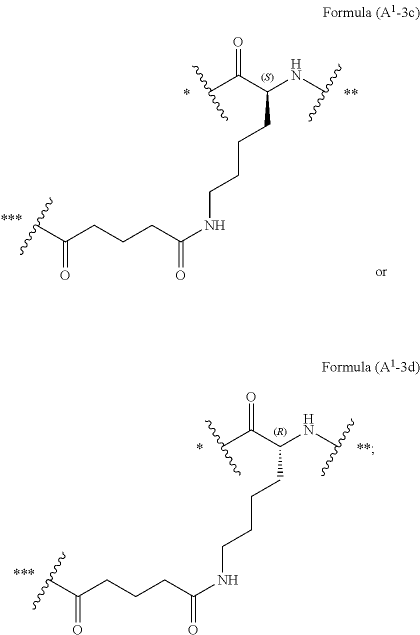

wherein * denotes a bond to L¹, ** denotes a bond to L², *** denotes a bond to L³.

In some embodiments, A¹ is of Formula (A¹-4a):

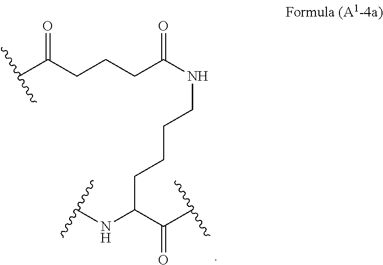 [SMILES: *NC(CCCCNC(=O)CCCC(*)=O)C(*)=O]

In some embodiments, A¹ is of Formula (A¹-4b):

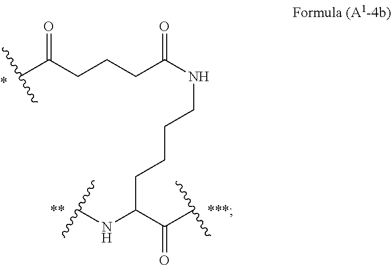 [SMILES: *NC(CCCCNC(=O)CCCC(*)=O)C(*)=O]

wherein * denotes a bond to L¹, ** denotes a bond to L², *** denotes a bond to L³.

In some embodiments, A¹ is of Formula (A¹-4c) or Formula (A¹4d):

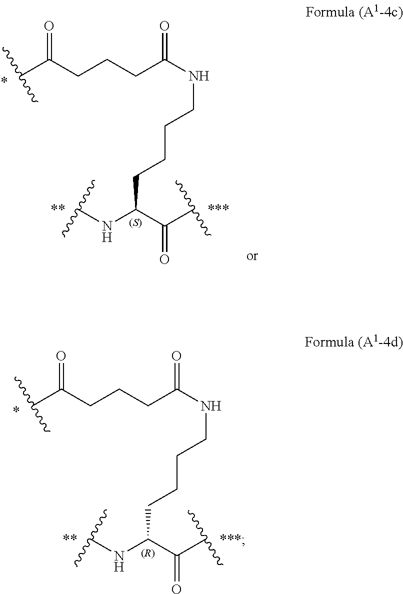

wherein * denotes a bond to L¹, ** denotes a bond to L², *** denotes a bond to L³.

### Enzymatically-Cleavable Linker (EL)

In an aspect, provided herein is a compound, or a pharmaceutically acceptable salt thereof, or a stereoisomer or mixture of stereoisomers thereof, having a structure represented by any one of Formulae (01)-(05):

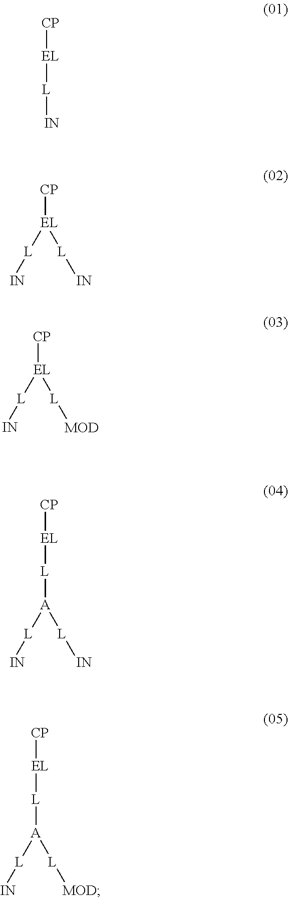

wherein:
CP is a cytotoxic or cytostatic group;
EL is an enzymatically-cleavable linker;
L is a stable linker;
A is a branching group;
IN is an integrin binding group; and
MOD is a physicochemical or pharmacokinetic modulator group.

In some embodiments, EL is a dipeptide having the formula -AA¹-AA²-, or a tripeptide having the formula -AA¹-AA²-AA³-, wherein each AA¹, AA², and AA³ is independently an amino acid, or a derivative thereof. In some embodiments, EL further comprises a self-immolative linker (SIL).

In some embodiments, provided herein is a compound of any one of Formulae (01)-(05), wherein EL has the formula:

*-AA¹-AA²-(AA³)₀₋₁-(SIL)₀₋₁-**;
wherein:
each AA¹, AA², and AA³ is independently an amino acid, or a derivative thereof,
SIL is a self-immolative linker,
* is a bond to L (e.g., L¹, L⁴, L⁵, L⁶, or L⁷); and
** is a bond to CP.

In some embodiments, EL is:
*-AA¹-AA²-AA³-SIL-**, *-AA¹-AA²-AA³-**
*-AA¹-AA²-SIL-**, or *-AA¹-AA²**;
   wherein * is a bond to L (e.g., L¹, L⁴, L⁵, or L⁷); and ** is a bond to CP

In some embodiments, SIL is a para-amino carbamate (PABC) group. In some embodiments, SIL is a group according to Scheme 1.

In some embodiments, EL is a dipeptide having the formula -AA¹-AA²-, wherein each AA¹ and AA² is independently an amino acid.

In some embodiments, the present invention provides compounds having a structure of formulae disclosed herein, or a pharmaceutically acceptable salt thereof; or a stereoisomer or mixture of stereoisomers thereof; wherein EL is a tripeptide, having the formula -AA¹-AA²-AA³-, wherein each AA¹, AA², and AA³ is independently an amino acid.

In some embodiments, the present invention provides compounds having a structure of formulae disclosed herein, or a pharmaceutically acceptable salt thereof; or a stereoisomer or mixture of stereoisomers thereof; wherein EL is cleaved by an enzyme from the class of proteases.

In some embodiments, the present invention provides compounds having a structure of formulae disclosed herein, or a pharmaceutically acceptable salt thereof; or a stereoisomer or mixture of stereoisomers thereof; wherein EL is cleaved by neutrophil elastase.

In some embodiments, the present invention provides compounds having a structure of formulae disclosed herein, or a pharmaceutically acceptable salt thereof; or a stereoisomer or mixture of stereoisomers thereof; wherein EL is cleaved by neutrophil elastase.

In some embodiments, the present invention provides compounds having a structure of formulae disclosed herein, or a pharmaceutically acceptable salt thereof; or a stereoisomer or mixture of stereoisomers thereof; wherein EL has the formula: L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu-. In some embodiments, EL having the formula L-Asp-L-Pro-L-Val, L-Asn-L-Pro-L-Val, -Gly-L-Pro-L-Val-, L-Ala-L-Pro-L-Val-, L-Nva-L-Pro-L-Val-, L-His-L-Pro-L-Val-, L-Asp-L-Pro-L-Ile, L-Asn-L-Pro-L-Ile, -Gly-L-Pro-L-Ile-, L-Ala-L-Pro-L-Ile-, L-Nva-L-Pro-L-Ile-, L-His-L-Pro-L-Ile-, L-Asp-L-Pro-L-Leu, L-Asn-L-Pro-L-Leu, -Gly-L-Pro-L-Leu-, L-Ala-L-Pro-L-Leu-, L-Nva-L-Pro-L-Leu-, or L-His-L-Pro-L-Leu- is cleavable by neutrophil elastase.

In some embodiments, EL has the formula L-Asp-L-Pro-L-Val, L-Asn-L-Pro-L-Val, -Gly-L-Pro-L-Val-, L-Ala-L-Pro-L-Val-, L-Nva-L-Pro-L-Val-, or L-His-L-Pro-L-Val-. In some embodiments, EL has the formula L-Asp-L-Pro-L-Val, L-Asn-L-Pro-L-Val, or -Gly-L-Pro-L-Val-.

In some embodiments, EL is of Formula (EL-1a):

L-Asp-L-Pro-L-Val Formula (EL-1a).

In some embodiments, EL is of Formula (EL-1b):

L-Asp*-L-Pro-L-Val Formula (EL-1b)
wherein Asp* represents a prodrug of aspartate, such as an alkyl ester of Asp, wherein the alkyl group is substituted with an amine (e.g., NH₂, N(CH₃)₂, N(CH₃)₃^{m}) or a linker to an integrin binder (e.g., -L⁶-IN, NHL⁶-IN, or NHCO-IN). In some embodiments, Asp* represents an alkyl ester of Asp, wherein the alkyl group is substituted with a carboxylic acid (COOH). In some embodiments, Asp* represents an alkyl ester of Asp, wherein the alkyl group is substituted with a physicochemical or pharmacokinetic modulating group (MOD). In some embodiments, MOD is —COOH, —COONa—COOCH₃, —NH₂, —N(CH₃)₂, —N(CH₃)₃, —NC(═NH)NH₂, —OH, or —OCH₃. In some embodiments, MOD is —COOH. In some embodiments, MOD is —OH. In some embodiments, MOD is —H or —CH₃.

In some embodiments, EL is of Formula (EL-2):

L-Asn-L-Pro-L-Val Formula (EL-2).

In some embodiments, EL is of Formula (EL-3):

Gly-L-Pro-L-Val Formula (EL-3).

In some embodiments, EL is of Formula (EL-4):

L-Asp-L-Pro-L-Ala Formula (EL-4).

In some embodiments, EL is of Formula (EL-5):

L-Asn-L-Pro-L-Ala Formula (EL-5).

In some embodiments, EL is of Formula (EL-6):

Gly-L-Pro-L-Ala Formula (EL-6).

In some embodiments, EL is represented by Formula (EL-I):

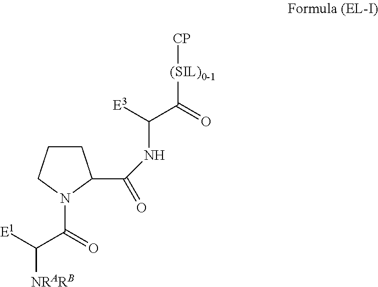

wherein
CP is a cytotoxic or cytostatic group;
SIL is a self-immolative group;
E¹ is hydrogen, —CH₃, —CH₂CH₃, —CH₂CH₂CH₃, —CH₂C(O)NH₂, —CH₂C(O)OH, —CH₂C(O)OR¹;
E³ is CH₃, CH(CH₃)₂, CH₂CH(CH₃)₂, or CH(CH₃)CH₂CH₃;
R^{A} is hydrogen or C₁₋₆ alkyl; and
R^{B} is a bond to a spacer (SP) as described herein, or linker (L¹, L⁴, L⁵, or L⁷).

In some embodiments, EL is a peptide group having the formula Val-Pro-Asp, Val-Pro-Asp*, Val-Pro-Asn, Val-Pro-Gly, or Ala-Pro-Asp. In some embodiments, EL is a peptide group having the formula L-Val-L-Pro-L-Asp, L-Val-L-Pro-L-Asn, L-Val-L-Pro-Gly, or L-Ala-L-Pro-L-Asp. In some embodiments, EL is a peptide group having the formula L-Val-L-Pro-L-Asp. In some embodiments, EL is a peptide group having the formula L-Val-L-Pro-L-Asp*. In some embodiments, EL is a peptide group having the formula L-Val-L-Pro-L-Asn. In some embodiments, EL is a peptide group having the formula L-Val-L-Pro-L-Gly. In some embodiments, EL is a peptide group having the formula Gly-L-Pro-L-Asp.

In some embodiments, EL is an enzymatically-cleavable group. In some embodiments, EL is a neutrophil elastase-cleavable group. In some embodiments, EL is an enzymatically cleavable tripeptide. In some embodiments, EL is a tripeptide group cleavable by neutrophil.

In some embodiments, EL is a peptide group having the formula:
Val-Pro-Asp, Val-Pro-Asp*, Val-Pro-Asn, Val-Pro-Gly,
Ala-Pro-Asp, Ala-Pro-Asp*, Ala-Pro-Asn, or Ala-Pro-Gly.

In some embodiments, EL is:

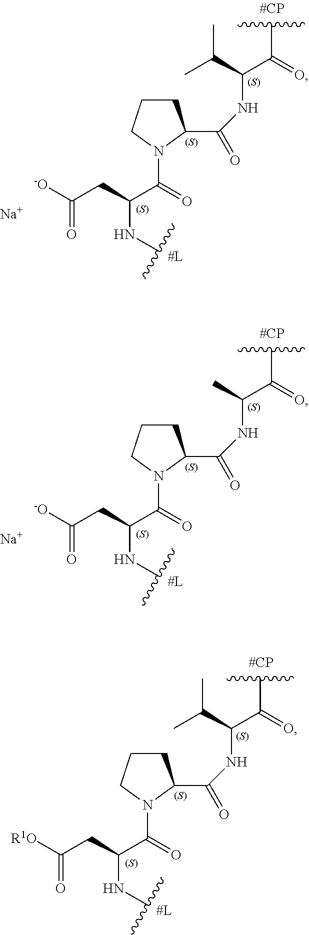

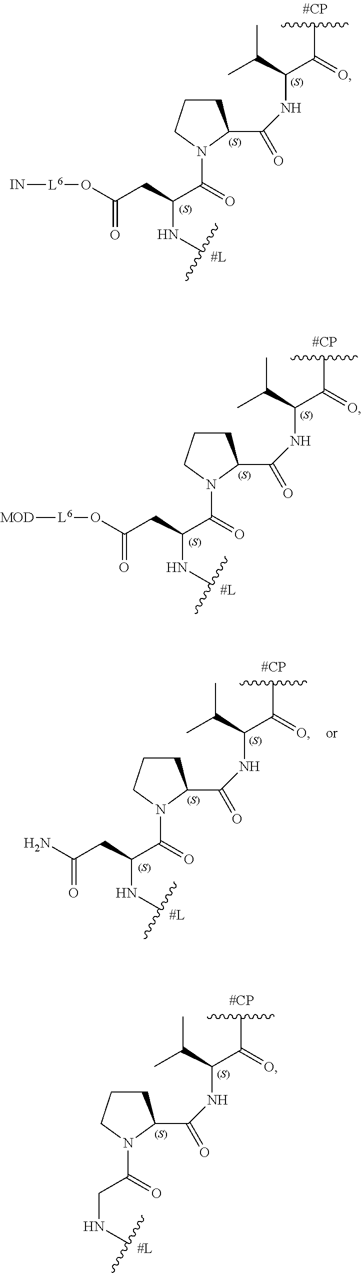

wherein #CP denotes a bond to CP, and #L denotes a bond to a linker L (e.g., L¹, L⁴, L⁵, or L⁷), R¹ is as defined in Formula (I) or Formula (II), L⁶ is a linker, IN is an integrin binder, MOD is a physicochemical or pharmacokinetic modulating group (e.g., a polar and/or ionizable group).

### Physicochemical or Pharmacokinetic Modulators (MOD)

In some embodiments, the present disclosure provides conjugates comprising a physicochemical or pharmacokinetic modulator (“MOD”). In some embodiments, MOD is a physicochemical modulator. In some embodiments, MOD is a pharmacokinetic modulator. Generally, these terms are used interchangeably unless otherwise specified. A group MOD may be connected to the rest of the conjugate via a linker (e.g., a stable liner). In some embodiments, MOD is connected to EL via a linker (e.g., L⁶). In some embodiments, MOD is bonded to A¹ via a linker (e.g., L³). In some embodiments, MOD is a charged or a polar group. Examples of charged or polar groups include hydroxides and alcohols, carboxylates and carboxylic acids, aminiums and amines, guanidiniums and guanidines, and the like. In some embodiments, MOD is or comprises an amine. In some embodiments, MOD is a polyamine group. In some embodiments, MOD is —NR₂ or —NR₃⁺. In some embodiments, MOD is —C₁₋₆ alkyl-NR₂ or —C₁₋₆ alkyl-NR₃⁺(e.g., —C₁₋₆ alkyl-NH₂, —C₁₋₆ alkyl-NHCH₃, —C₁₋₆ alkyl-N(CH₃)₂, or —C₁₋₆ alkyl-N(CH₃)₃⁺). In some embodiments, MOD is —C₁₋₆ alkyl-NHC(═NH⁺)NH₂. In some embodiments, MOD is a polyacid group. In some embodiments, MOD is —COOH or —COO⁻. In some embodiments, MOD is —C₁₋₆ alkyl-COOR (e.g., —C₁₋₆ alkyl-COOH, —C₁₋₆ alkyl-COO⁻, —C₁₋₆ alkyl-COO⁻Na⁺, or —C₁₋₆ alkyl-COOCH₃.). In some embodiments, MOD is a group that is converted to a polar or charged group in vivo (e.g., MOD is an alkyl ester, which is cleaved in vivo to an alkyl acid, thereby reducing membrane permeability). In some embodiments, MOD is —OH or —O⁻. In some embodiments, MOD is an amino acid. In some embodiments, MOD is a basic amino acid. In some embodiments, MOD is Arg, His, or Lys. In some embodiments, MOD is an acidic amino acid. In some embodiments, MOD is Glu or Asp. In some embodiments, MOD is an unnatural amino acid (e.g., D-Glu or D-Asp). In some embodiments, MOD is a polar amino acid (e.g., Ser, Thr, Asn, Gln). In some embodiments, provided herein is a compound of the formula:

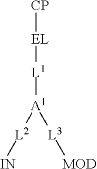

wherein:
CP is a cytotoxic or cytostatic group;
EL is an enzymatically-cleavable linker;
L¹, L², and L³ are linkers;
A is a branching group;
IN is an integrin binding group; and
MOD is a physicochemical or pharmacokinetic modulator group.

In some embodiments, L³ is a heteroalkyl linker. In some embodiments, L³ is a simple spacer. In some embodiments, L³ is a carbonyl, an acyl linker, an amide linker, or an alkyl linker. In some embodiments, L³ is an optionally substituted alkyl linker (e.g., an optionally substituted alkyl amide linker).

In some embodiments, L³ is —(CO)ᵣ(CH₂)ₛ(NR¹⁰C(O)C₁₋₆ alkyl)ₜ(NR¹¹)ᵤ(CO)ᵥ—.

In some embodiments, L³ is —[N(CH₃)CH₂C(O)]₁₋₁₂ and MOD is —OH or —O⁻. Examples of L³-MOD include: —[N(CH₃)CH₂C(O)]₄O⁻, —[N(CH₃)CH₂C(O)]₅O⁻, —[N(CH₃)CH₂C(O)]₆O⁻, —[N(CH₃)CH₂C(O)]₇O⁻, —[N(CH₃)CH₂C(O)]₈O⁻, —[N(CH₃)CH₂C(O)]₉O⁻, and —[N(CH₃)CH₂C(O)]₁₀O⁻.

In some embodiments, MOD is —COOH, —COO⁻Na⁺, —COOCH₃, —NH₂, —N(CH₃)₂, —N(CH₃)₃, —NC(═NH)NH₂, —OH, —O—Na⁺, or —OCH₃. In some embodiments, MOD is —OH, —COOH, —NH₂, NHCH₃, —N(CH₃)₂, —N(CH₃)₃⁺, or a salt thereof. In some embodiments, MOD is —COOH. In some embodiments, MOD is —OH. In some embodiments, MOD is —H or —CH₃. In some embodiments, L³ is C₁₋₁₂ alkyl. In some embodiments, L³ is NHC₁₋₁₂ alkyl. In some embodiments, L³ is NHC(O)—C₁₋₁₂ alkyl. In some embodiments, L³ is C(O)—C₁₋₁₂ alkyl. In some embodiments, L³ is a linker (L) or spacer (SP) as defined herein.

### Integrin Binders (IN)

In some embodiments, the present invention provides compounds having a structure of formulae disclosed herein, or a pharmaceutically acceptable salt thereof; or a stereoisomer or mixture of stereoisomers thereof; wherein IN is an integrin binder comprising peptides, proteins, antibodies, or small molecules. In some embodiments, molecular weight of such peptides, proteins, antibodies, or small molecules is <1 kD.

In some embodiments, the present invention provides compounds having a structure of formulae disclosed herein, or a pharmaceutically acceptable salt thereof; or a stereoisomer or mixture of stereoisomers thereof; wherein IN is a peptidic or peptidomimetic integrin binder.

In some embodiments, the present invention provides compounds having a structure of formulae disclosed herein, or a pharmaceutically acceptable salt thereof; or a stereoisomer or mixture of stereoisomers thereof; wherein IN is an integrin binder comprising non-peptides, non-antibodies, or small molecules. In some embodiments, molecular weight of such non-peptides, non-proteins, non-antibodies, or small molecules (e.g., <1 kD). In some embodiments, IN is a small molecule integrin binder. In some embodiments, IN is a small molecule integrin binder having two or more urea groups (—NHC(O)NH—). In some embodiments, IN is a small molecule α_{b}β₃ integrin binder.

In some embodiments, the present invention provides compounds having a structure of formulae disclosed herein, or a pharmaceutically acceptable salt thereof; or a stereoisomer or mixture of stereoisomers thereof; wherein IN is a linear integrin binding peptide.

In some embodiments, IN is a macrocyclic integrin binding peptide.

In some embodiments, the linear integrin binding peptide is a constrained macrocyclic integrin binding peptide.

In some embodiments, IN is a non-peptidic or non-peptidomimetic integrin binder.

In some embodiments, IN is a tumor-binding moiety. In some embodiments, IN binds to both tumor and non-tumor cells. In some embodiments, IN binds rapidly dividing cells (e.g., tumor cells or other hyperproliferating cells).

In some embodiments, IN is an αᵥβ₃-binding moiety.

In some embodiments, IN is IN-1a or IN-1b, wherein

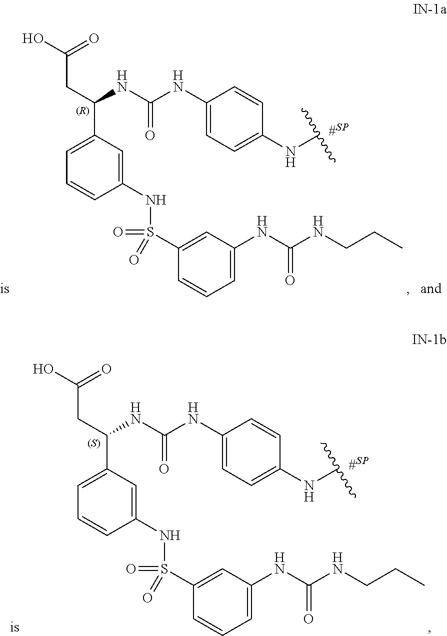

or a pharmaceutically acceptable salt thereof; or a stereoisomer or mixture of stereoisomers thereof, wherein #SP denotes a bond to a spacer, e.g., one of L², L³, L⁴, L⁵, L⁶, or L⁷.

In some embodiments, IN has a structure:

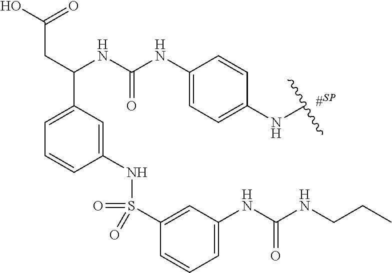 [SMILES: *Nc1ccc(NC(=O)NC(CC(=O)O)c2cccc(NS(=O)(=O)c3cccc(NC(=O)NCCC)c3)c2)cc1]

or a pharmaceutically acceptable salt thereof; or a stereoisomer or mixture of stereoisomers thereof; wherein #^{SP} denotes a bond to a spacer, e.g., one of L², L³, L⁴, L⁵, L⁶, or L⁷.

In some embodiments, IN is IN-2a or IN-2b:

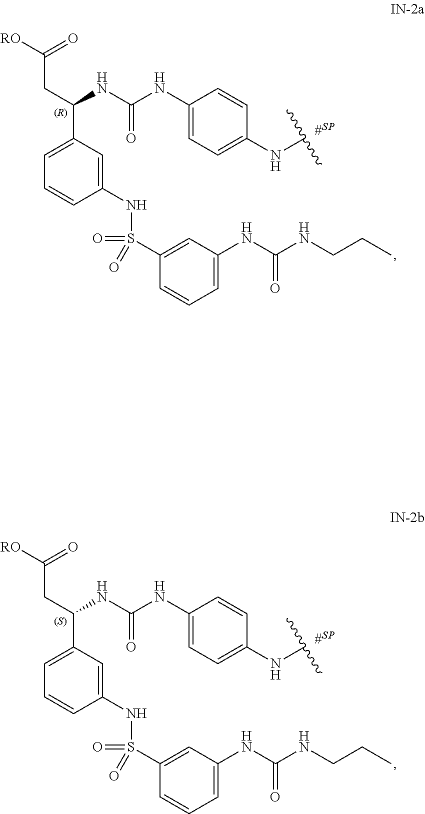

or a pharmaceutically acceptable salt thereof, or a stereoisomer or mixture of stereoisomers thereof; wherein #SP denotes a bond to a spacer, e.g., one of L², L³, L⁴, L⁵, L⁶, or L⁷, and R is a substituted or unsubstituted alkyl; wherein if R is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), —SO₂N(C₁₋₆alkyl)₂ and -MOD. In some embodiments, R is C₁₋₁₀ alkylamine. In some embodiments, R is C₁₋₁₀ alkyl substituted with), —NH₂, —NH(C₁₋₆alkyl), —N(C₁₋₆alkyl)₂, —N(C₁₋₆alkyl)₃⁺. In some embodiments, R is —(CH₂)₁₋₁₀—NH₂, —(CH₂)₁₋₁₀—N(CH₃)₂, —(CH₂)₁₋₁₀—N(CH₃)₃⁺, or —(CH₂)₁₋₁₀—NHC(O)-IN.

In some embodiments, IN has a structure:

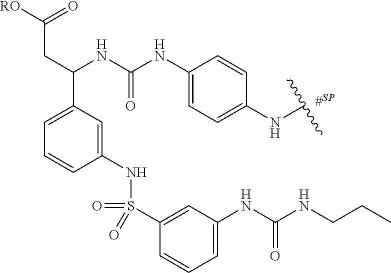

or a pharmaceutically acceptable salt thereof, or a stereoisomer or mixture of stereoisomers thereof; wherein #^{SP} denotes a bond to a spacer, e.g., one of L², L³, L⁴, L⁵, L⁶, or L⁷; and R is a substituted or unsubstituted alkyl; wherein if R is substituted, it is substituted with one or more groups independently selected from deuterium, halogen, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), -MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂. In some embodiments, R is a substituted or unsubstituted C₁₋₁₂ alkylamine.

### Cytotoxic or Cytostatic Radicals (CP)

Disclosed herein are compounds having a cytotoxic or cytostatic radical, CP. In some embodiments, CP is a polypeptide (e.g., a protein, an antibody) having a tertiary or quaternary structure. In some embodiments, CP is a polypeptide having no tertiary or quaternary structure (e.g., a flexible peptide, conformationally constrained peptide, a stapled peptide, a helical peptide, and the like). In some embodiments, CP is non-peptidic. In some embodiments, CP is a small molecule radical (e.g., <1 kD in molecular weight). In some embodiments, CP is a macrocyclic radical. In some embodiments, CP is a polycyclic radical. In some embodiments, CP is a polycyclic radical having four or five rings. In some embodiments, CP is a polycyclic radical having five fused rings. In some embodiments, CP is a pentacyclic radical. In some embodiments, CP is a pentacyclic radical having an alpha-hydroxy lactone moiety in one of the rings. In some embodiments, CP is represented by the structure:

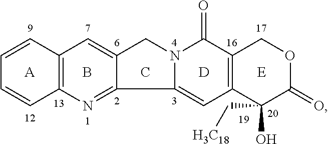

wherein the radical is a hydroxy radical in the C20 position. In some embodiments, CP is a C20 hydroxy radical of

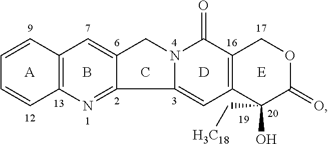

wherein the C7 position is substituted with an ethyl group. In some embodiments, CP is a camptothecin radical, or a derivative thereof. In some embodiments, CP is a 7-ethyl camptothecin radical. In some embodiments, CP is a camptothecin radical or a 7-ethyl camptothecin radical, which forms an ester linkage at the C20 hydroxyl group to an amino acid (e.g., an amino acid of EL or Formula (I)). In some embodiments, CP is a camptothecin or 7-ethyl camptothecin radical bonded via an ester linkage to the carbonyl of a valine residue. In some embodiments, CP is

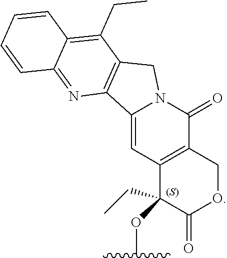

Any combination of the groups described above for the various variables is contemplated herein. Throughout the specification, groups and substituents thereof are chosen by one skilled in the field to provide stable moieties and compounds.

### Pharmaceutically Acceptable Salts

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, pharmaceutically acceptable salts are obtained by reacting a conjugate described herein with an acid. In some embodiments, the conjugate described herein (e.g., free base form) is basic and is reacted with an organic acid or an inorganic acid. Inorganic acids include, but not limited to, hydrochloric acid, hydrobromic acid, sulfuric acid, phosphoric acid, nitric acid, and metaphosphoric acid. Organic acids include, but not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts of the inventive compounds especially include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, benzenesulphonic acid, toluenesulphonic acid, naphthalenedisulphonic acid, formic acid, acetic acid, trifluoroacetic acid, propionic acid, succinic acid, fumaric acid, maleic acid, lactic acid, tartaric acid, malic acid, citric acid, gluconic acid, benzoic acid, and embonic acid.

In some embodiments, a conjugate described herein is prepared as a chloride salt, sulfate salt, bromide salt, mesylate salt, maleate salt, citrate salt or phosphate salt.

In some embodiments, pharmaceutically acceptable salts are obtained by reacting a conjugate described herein with a base. In some embodiments, the conjugate described herein is acidic and is reacted with a base. In such situations, an acidic proton of the conjugate described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt. In some embodiments, the compounds provided herein are prepared as a sodium salt, e.g., a monosodium salt, a disodium salt, a trisodium salt, or a tetrasodium salt. In some embodiments, the salt is a disodium or trisodium salt. In some embodiments, the salt is a trifluoroacetate (TFA) salt, e.g., a mono-TFA, di-TFA, tri-TFA, or tetra-TFA salt.

In some embodiments, pharmaceutically acceptable salts of the inventive compounds also include salts derived from conventional bases, by way of example alkali metal salts (e.g. sodium and potassium salts), alkaline earth metal salts (e.g. calcium and magnesium salts), zinc salts and ammonium salts derived from ammonia or organic amines having 1 to 20 carbon atoms, by way of example ethylamine, diethylamine, triethylamine, N,N-ethyldiisopropylamine, monoethanolamine, diethanolamine, triethanolamine, dimethylaminoethanol, diethylaminoethanol, tris(hydroxymethyl)aminomethane, choline, benzalkonium, procaine, dibenzylamine, dicyclohexylamine, N-methylmorpholine, N-methylpiperidine, arginine, lysine, and 1,2-ethylenediamine.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, invention is described as the forms of the inventive compounds which form a complex in the solid or liquid state by coordination with solvent molecules. Hydrates are a specific form of the solvates in which the coordination is with water. Solvates preferred in the context of the present invention are hydrates.

The methods and formulations described herein include the use of N-oxides (if appropriate), or pharmaceutically acceptable salts of compounds having the structure of any one of the formulae disclosed herein, as well as active metabolites of these compounds having the same type of activity.

In some embodiments, sites on the organic radicals (e.g. alkyl groups, aromatic rings) of compounds of formulae disclosed herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug or substrate tissue distribution assays. In one aspect, substitution with isotopes such as deuterium affords certain therapeutic advantages resulting from greater metabolic stability, such as, for example, increased in vivo half-life or reduced dosage requirements.

In some embodiments, the present invention also encompasses all suitable isotopic variants of the inventive compounds. An isotopic variant of an inventive compound is understood here to mean a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulfur, fluorine, chlorine, bromine and iodine, such as 2H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, particularly compounds labelled with ³H, ¹⁴C, or ¹⁸F isotopes are suitable for the purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore possibly also constitute a preferred embodiment of the present invention. Isotopic variants of the inventive compounds can be prepared by commonly used processes known to those skilled in the art, for example by the methods described further down and the procedures described in the working examples, by using corresponding isotopic modifications of the respective reagents or starting compounds.

In some embodiments, the compounds disclosed herein possess one or more stereocenters and each stereocenter exists independently in either the R or S configuration. In some embodiments, the conjugate described herein exists in the R configuration. In some embodiments, the conjugate described herein exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

### Prodrugs

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In some embodiments, a cytotoxic or cytostatic prodrug (e.g., a topoisomerase inhibitor prodrug (e.g., a camptothecin or 7-ethyl camptothecin prodrug) disclosed herein is to increase the therapeutic window of cytotoxic or cytostatic moiety and achieve a tumor targeting of this class of anti-tumor compounds. The inventive prodrug conjugates consist of an αᵥβ3 integrin binding moiety which is linked to camptothecin moiety via a peptide linker, which is cleavable by proteases present in the tumor microenvironment to release the parent camptothecin compound at the site of action. Enzymes present in the tumor microenvironment include, but not limited to, neutrophil elastase. A prodrug may be converted into the parent drug in vivo via an enzymatic or a chemical process.

In some embodiments, a prodrug is activated upon enzymatic cleavage (e.g., a tumor-associated enzyme such as neutrophil elastase). In some embodiments, the prodrug contains a peptide sequence that is recognized by a given enzyme. In some embodiments, a prodrug is selectively cleaved by a specific enzyme.

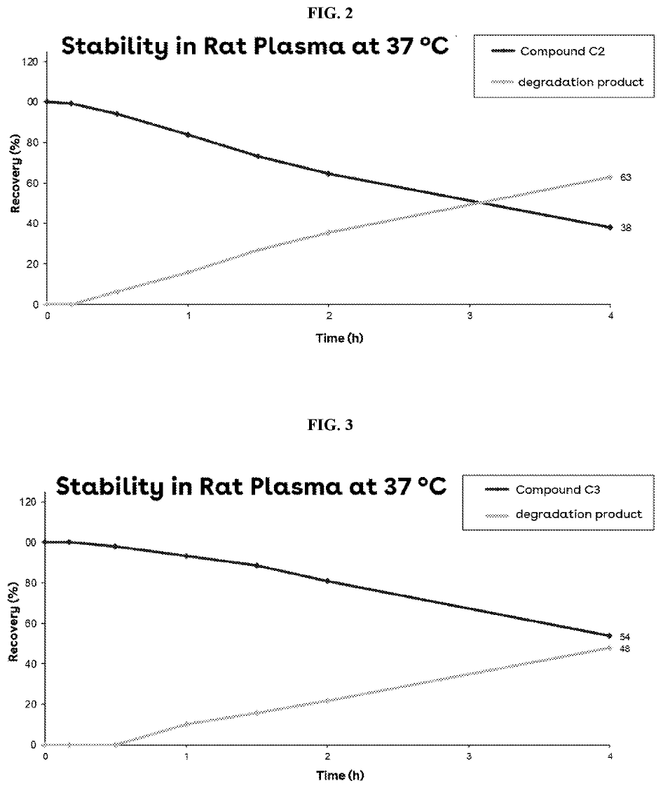

In some embodiments, a prodrug further comprises a non-peptidic prodrug moiety (e.g., an ester) which is released in vivo. Non-peptidic prodrug moiety indicates that the bond being metabolized or broken to release the active agent is not a peptide bond (—C(O)—NH—). In some embodiments, the non-peptidic prodrug moiety is an alkyl ester (including substitutions such as alkyl substitutions on the alkyl group). In some embodiments, the non-peptidic prodrug moiety is an ester of an amino acid, such as an aspartate or glutamate residue. In some embodiments, the alkyl ester prodrug is indicated by Asp* or Glu*, indicating an alkyl (e.g., substituted alkyl) group is masking the carboxylic acid moiety of the side-chain. In some embodiments, provided herein are prodrug compounds wherein an alkylamine (—C₁₋₆alkyl-NR₂) or alkylaminium (—C₁₋₆alkyl-NR₃⁺) group is released in vivo (where “R” as used here is hydrogen or C₁₋₆alkyl) from an amino acid ester (e.g., Asp* or Glu*) to liberate the acid moiety. In some embodiments, a non-peptidic prodrug moiety (e.g., an ester (e.g., an alkylamine or alkylaminium ester)) is released (i.e., cleaved) in plasma, while an enzymatically cleavable moiety remains intact (e.g., when the cleaving enzyme is not present or abundant). In some embodiments, the prodrug ester is slowly cleaved in plasma with no detectable release of the enzymatically cleavable moiety or the payload (e.g., the cytotoxic or cytostatic ligand) attached thereto. In some embodiments, the non-peptidic (i.e., ester) prodrug moiety is cleaved before the peptidic prodrug moiety. In some embodiments, the non-peptidic (i.e., ester) prodrug moiety is cleaved independently of protease activity (e.g., non-proteolytic cleavage). In some embodiments, the non-peptidic (i.e., ester) prodrug moiety is cleaved to a degree of about 50% (+5%) slowly. As used here, “slowly” means the prodrug is cleaved to a degree of about 50% after at least two hours in plasma. In some embodiments, slow cleavage constitutes about 50% prodrug release (i.e., release of the ester moiety to yield the active carboxylic acid) after about 2, about 3, about 4, about 5, about 6, about 8, about 10, or about 12 hours in plasma. In some embodiments, slow cleavage constitutes about 50% prodrug release after about 2 hours to about 12 hours in plasma. In some embodiments, an ester prodrug is cleaved to a degree of about 50% after about 2 hours to about 6 hours in plasma. In some embodiments, an ester prodrug is cleaved to a degree of about 50% after about 2 hours to about 4 hours in plasma. As described previously, the non-peptidic (i.e., ester) prodrug moiety is cleaved independently (e.g., in the absence or presence) of proteolytic enzymes such as cathepsin B, legumain, or neutrophil elastase. As shown in FIG. 2 and FIG. 3, in some embodiments, the ester prodrug moiety is cleaved to a degree of about 50% after about 3 or 4 hours (and within about 6 hours) of administration in vivo. The degradation product of such a prodrug is, in some embodiments, the parent conjugate sans alkyl ester, meaning the enzymatically cleavable portion, cytotoxic or cytostatic moiety, linker/spacer, and integrin binder remain intact. In some embodiments, enzymatic cleavage takes place after release of the ester prodrug moiety. In some embodiments, the protease responsible for cleaving the enzymatically cleavable moiety recognizes the free Asp or Glu residue, but not the Asp* or Glu* prodrug ester.

Prodrugs of the compounds described herein include, but not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (e.g., the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a conjugate described herein as set forth herein are included within the scope of the claims. In some cases, some of the herein-described compounds is a prodrug for another derivative or active compound.

In some embodiments, any one of the hydroxyl group(s), amino group(s), or carboxylic acid group(s) are functionalized in a suitable manner to provide a prodrug moiety. In some embodiments, the prodrug moiety is as described above.

### Pharmaceutical Compositions

The present invention provides a pharmaceutical composition comprising a compound of formulae disclosed herein, or a pharmaceutically acceptable salt thereof; or a stereoisomer or mixture of stereoisomers thereof; and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated to provide slow or controlled release of the active ingredient therein. All formulations for oral administration should be in dosages suitable for such administration. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal, and intramuscular administration.

Pharmaceutical compositions suitable for topical administration include liquid or semi-liquid preparations suitable for penetration through the skin to the site of inflammation such as gels, liniments, lotions, creams, ointments, or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In addition to the ingredients particularly mentioned above, the compounds and compositions described herein may include other agents conventional in the art having regard to the type of formulation in question, for example those suitable for oral administration may include flavoring agents.

### Compounds for Use in Treating Diseases or Disorders

In some embodiments, the present invention provides compounds of formulae disclosed herein, or a pharmaceutically acceptable salt thereof; or a stereoisomer or mixture of stereoisomers thereof; for the treatment of a disease or disorder.

In some embodiments, the present invention provides compounds of formulae disclosed herein, or a pharmaceutically acceptable salt thereof; or a stereoisomer or mixture of stereoisomers thereof; for use in the manufacture of a medicament for treating a disease or disorder described herein.

In some embodiments, the present invention provides compounds of formulae disclosed herein, or a pharmaceutically acceptable salt thereof; or a stereoisomer or mixture of stereoisomers thereof; for the treatment of a hyperproliferative disorder.

In some embodiments, the present invention provides compounds of formulae disclosed herein, or a pharmaceutically acceptable salt thereof; or a stereoisomer or mixture of stereoisomers thereof; wherein for the treatment of an autoimmune disorder.

In some embodiments, the present invention provides compounds of formulae disclosed herein, or a pharmaceutically acceptable salt thereof; or a stereoisomer or mixture of stereoisomers thereof; for the treatment of an ophthalmological disease or disorder. In some embodiments, the ophthalmological disease or disorder is macular degeneration. In some embodiments, the ophthalmological disease or disorder is an ocular and/or intraocular malignancy. In some embodiments, the ocular/intraocular malignancy is uveal melanoma.

In some embodiments, the present invention provides compounds of formulae disclosed herein, or a pharmaceutically acceptable salt thereof; or a stereoisomer or mixture of stereoisomers thereof; wherein for the treatment of a skin disorder. In some embodiments, the skin disorder is psoriasis.

In some embodiments, the present invention provides compounds of formulae disclosed herein, or a pharmaceutically acceptable salt thereof; or a stereoisomer or mixture of stereoisomers thereof; for the treatment of a cancer. In some embodiments, the cancer is an invasive cancer. In some embodiments, the cancer is a metastatic cancer. In some embodiments, the cancer is of the breast, respiratory tract, brain, reproductive organs, digestive tract, urinary tract, eye, liver, skin, head and neck, thyroid, parathyroid or a metastasis thereof.

In some embodiments, the cancer of the breast is invasive ductal carcinoma, invasive lobular carcinoma, ductal carcinoma in situ, lobular carcinoma in situ, inflammatory breast cancer, basal breast cancer, or triple negative breast cancer.

In some embodiments, the cancer of the respiratory tract is small-cell, non-small-cell lung carcinoma, bronchial adenoma, or pleuropulmonary blastoma.

In some embodiments, the cancer of the brain is a brain stem glioma, hypothalmic glioma, glioblastoma, cerebellar astrocytoma, cerebral astrocytoma, medulloblastoma, ependymoma, neuroectodermal tumor, or pineal tumor.

In some embodiments, the cancer of the reproductive organs is a prostate cancer, testicular cancer, endometrial cancer, cervical cancer, ovarian cancer, vaginal cancer, vulvar cancer, or a sarcoma of the uterus.

In some embodiments, the cancer of the digestive tract is anal cancer, colon cancer, colorectal cancer, esophageal cancer, gallbladder cancer, gastric cancer, pancreatic cancer, rectal cancer, small intestinal cancer, or salivary gland cancer.

In some embodiments, the cancer of the urinary tract is bladder cancer, penile cancer, kidney cancer, renal pelvis cancer, ureter cancer, or urethral cancer.

In some embodiments, the cancer of the eye is intraocular melanoma or retinoblastoma.

In some embodiments, the cancer of the liver is to hepatocellular carcinoma, liver cell carcinoma with or without fibrolamellar variant, cholangiocarcinoma, intrahepatic bile duct carcinoma, or mixed hepatocellular cholangiocarcinoma.

In some embodiments, the cancer of the skin is squamous cell carcinoma, Kaposi's sarcoma, malignant melanoma, Merkel cell skin cancer, or non-melanoma skin cancer.

In some embodiments, the cancer of the head-and-neck is laryngeal cancer, hypopharyngeal cancer, nasopharyngeal cancer, oropharyngeal cancer, or a lip and oral cavity cancer.

In some embodiments, the cancer is a sarcoma. In some embodiments, the sarcoma is Ewing sarcoma, osteosarcoma, or fibrosarcoma. In some embodiments, the sarcoma is a sarcoma of the soft tissue, osteosarcoma, malignant fibrous histiocytoma, lymphosarcoma, or rhabdomyosarcoma

In some embodiments, the cancer is a lymphoma. In some embodiments, the lymphoma is AIDS-related lymphoma, non-Hodgkin's lymphoma, cutaneous T-cell lymphoma, Hodgkin's disease, or lymphoma of the central nervous system.

In some embodiments, the cancer is a leukemia. In some embodiments, the leukemia is acute myeloid leukemia, acute lymphoblastic leukemia, chronic lymphocytic leukemia, chronic myelogenous leukemia, or hairy cell leukemia.

In some embodiments, the cancer is breast cancer, colon cancer, renal cancer, or lung cancer.

### Methods of Dosing and Treatment Regimens

In some embodiments, the present invention provides a method of treating a disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the compound used in the method of treating a disease or disorder is a compound of Formula (A), Formula (B), Formula (C), Formula (D), Formula (I), Formula (I-A), Formula (I-B), Formula (I-C), Formula (I-D), Formula (II), Formula (II-A), Formula (II-B), Formula (II-C), Formula (II-D), Formula (III-A), Formula (III-B), Formula (III-C), or Formula (III-D), or a pharmaceutically acceptable salt thereof, or a stereoisomer or mixture of stereoisomers thereof.

In some embodiments, the present invention provides a method of treating a hyperproliferative disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating an autoimmune disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating an ophthalmological disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating macular degeneration in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a skin disease or disorder in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating psoriasis in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating an invasive cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a metastatic cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the breast, respiratory tract, brain, reproductive organs, digestive tract, urinary tract, eye, liver, skin, head and neck, thyroid, parathyroid or a metastasis thereof in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the breast in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating an invasive ductal carcinoma, invasive lobular carcinoma, ductal carcinoma in situ, lobular carcinoma in situ, inflammatory breast cancer, basal breast cancer, or triple negative breast cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the respiratory tract in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a small-cell lung carcinoma, non-small-cell lung carcinoma, bronchial adenoma, or pleuropulmonary blastoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the brain in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a brain stem glioma, hypothalamic glioma, glioblastoma, cerebellar astrocytoma, cerebral astrocytoma, medulloblastoma, ependymoma, neuroectodermal tumor, or pineal tumor in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the reproductive organs in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating prostate cancer, testicular cancer, endometrial cancer, cervical cancer, ovarian cancer, vaginal cancer, vulvar cancer, or a sarcoma of the uterus in a subject, comprising administering a therapeutically effective amount of a compound of formulae disclosed herein, or a pharmaceutically acceptable salt thereof; or a stereoisomer or mixture of stereoisomers thereof, or a pharmaceutical composition thereof, to an individual in need thereof.

In some embodiments, the present invention provides a method of treating a cancer of the digestive tract in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating anal cancer, colon cancer, colorectal cancer, esophageal cancer, gallbladder cancer, gastric cancer, pancreatic cancer, rectal cancer, small intestinal cancer, or salivary gland cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the urinary tract in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating bladder cancer, penile cancer, kidney cancer, renal pelvis cancer, ureter cancer, or urethral cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the eye in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating intraocular melanoma or retinoblastoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the liver in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating hepatocellular carcinoma, liver cell carcinoma with or without fibrolamellar variant, cholangiocarcinoma, intrahepatic bile duct carcinoma, or mixed hepatocellular cholangiocarcinoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the skin in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating a squamous cell carcinoma, Kaposi's sarcoma, malignant melanoma, Merkel cell skin cancer, or non-melanoma skin cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a cancer of the head-and-neck in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating laryngeal cancer, hypopharyngeal cancer, nasopharyngeal cancer, oropharyngeal cancer, or a lip and oral cavity cancer in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating a sarcoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof. In some embodiments, the present invention provides a method of treating Ewing sarcoma, osteosarcoma, fibrosarcoma, sarcoma of the soft tissue, malignant fibrous histiocytoma, lymphosarcoma, or rhabdomyosarcoma in a subject, comprising administering a therapeutically effective amount of a compound of formulae disclosed herein, or a pharmaceutically acceptable salt thereof; or a stereoisomer or mixture of stereoisomers thereof; or a pharmaceutical composition thereof, to an individual in need thereof.

In some embodiments, the present invention provides a method of treating breast cancer, colon cancer, renal cancer, or lung cancer in a subject, comprising administering a therapeutically effective amount of a compound of formulae disclosed herein, or a pharmaceutically acceptable salt thereof; or a stereoisomer or mixture of stereoisomers thereof, or a pharmaceutical composition thereof, to an individual in need thereof.

In another aspect, provided herein is a method of treating a disease or disorder, the method comprising administering to a subject in need thereof a therapeutically effective amount of a pharmaceutical composition comprising any one of the compounds of the formulae disclosed herein, or a pharmaceutically acceptable salt thereof, or a stereoisomer or mixture of stereoisomers thereof, or a pharmaceutical composition thereof, to an individual in need thereof.

The total amount of the active compound to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The present invention further provides the use of the compound of the invention for the preparation of a pharmaceutical compositions for the treatment of the aforesaid disorders.

### Administration

It is possible for the compounds according to the invention to have systemic or local activity. For this purpose, they can be administered in a suitable manner, such as, for example, via the oral, parenteral, pulmonary, nasal, sublingual, lingual, buccal, rectal, vaginal, dermal, transdermal, conjunctival, optic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly or in a modified manner, such as, for example, tablets (e.g., uncoated or coated tablets, for example with enteric or controlled release coatings that dissolve with a delay or are insoluble), orally-disintegrating tablets, films/wafers, films/lyophilizates, capsules (e.g., hard or soft gelatin capsules), sugar-coated tablets, granules, pellets, powders, emulsions, suspensions, aerosols or solutions. It is possible to incorporate the compounds according to the invention in crystalline or amorphized or dissolved form into said dosage forms.

Parenteral administration can be affected with avoidance of an absorption step (for example intravenous, intraarterial, intracardial, intraspinal or intralumbal) or with inclusion of absorption (for example, intramuscular, subcutaneous, intracutaneous, percutaneous or intraperitoneal). Administration forms which are suitable for parenteral administration are, inter alia, preparations for injection and infusion in the form of solutions, suspensions, emulsions, lyophilizates or sterile powders. In some embodiments, a compound disclosed herein is administered via injection (e.g., parenteral injection). In some embodiments, a compound disclosed herein is formulated for injection. In some embodiments, a compound disclosed herein is formulated with a pharmaceutically acceptable excipient (e.g., a carrier or vehicle, e.g., an aqueous vehicle)) for injection. In some embodiments, the injection is intravenous. In some embodiments, the intravenous injection is an intravenous infusion.

Examples which are suitable for other administration routes are pharmaceutical forms for inhalation (e.g., powder inhalers, nebulizers, etc.), nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (e.g., lotions, mixturae agitandae, etc.), lipophilic suspensions, emulsions, ointments, creams, transdermal therapeutic systems (e.g., patches, etc.), milk, pastes, foams, dusting powders, implants or stents.

The compounds according to the invention can be incorporated into the stated administration forms. This can be affected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic©),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colorants (for example inorganic pigments such as, for example, iron oxides, titanium dioxide),
flavorings, sweeteners, flavor- or odor-masking agents.

The present invention furthermore relates to a pharmaceutical composition which comprise at least one compound according to the invention, conventionally together with one or more pharmaceutically suitable excipient(s), and to their use according to the present invention.

### Combination Treatments

In certain instances, it is appropriate to administer at least one conjugate described herein, or a pharmaceutically acceptable salt thereof, in combination with one or more other therapeutic agents.

In accordance with another aspect, the present invention covers pharmaceutical compositions, in particular, medicaments comprising at least one compound disclosed herein in the present invention and at least one or more further active ingredients, in particular, for the treatment or prophylaxis of a hyperproliferative disorder.

The compounds of the present invention can be administered as the sole pharmaceutical agent or in combination with one or more other pharmaceutically active ingredients where the combination causes no unacceptable adverse effects. The present invention also covers such pharmaceutical combinations. For example, the compounds of the present invention can be combined with known active ingredients for the treatment and/or prophylaxis of a hyperproliferative disorder.

Examples of active ingredients for the treatment and/or prophylaxis of a hyperproliferative disorder include, but not limited to, 131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, itraconazole, ixabepilone, ixazomib, lanreotide, lansoprazole, lapatinib, Iasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polatuzumab, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, venetoclax, vinblastine, vincristine, vindesine, vinflunine, vinorelbine, vismodegib, vorinostat, vorozole, yttrium-90 glass microspheres, zanubrutinib, zinostatin, zinostatin stimalamer, zoledronic acid, and zorubicin.

In one embodiment, the therapeutic effectiveness of one of the compounds described herein is enhanced by administration of an adjuvant (e.g., by itself the adjuvant has minimal therapeutic benefit, but in combination with another therapeutic agent, the overall therapeutic benefit to the patient is enhanced). In some embodiments, the benefit experienced by a patient is increased by administering one of the compounds described herein with another agent (which also includes a therapeutic regimen) that also has therapeutic benefit.

In one specific embodiment, a conjugate described herein, or a pharmaceutically acceptable salt thereof, is co-administered with a second therapeutic agent, wherein the conjugate described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The conjugates described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Definitions

Unless otherwise defined, all of the technical terms used herein have the same meaning as commonly understood by one of ordinary skill in the art in the field to which this disclosure belongs.

Unless otherwise stated, the following terms used in this application have the definitions given below.

The section headings used herein are for organizational purposes only and are not to be construed as limiting the subject matter described.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, unrecited additives, components, integers, elements or method steps.

As used herein, the term “about” a number refers to that number plus or minus 10% of that number. The term ‘about’ when used in the context of a range refers to that range minus 10% of its lowest value and plus 10% of its greatest value.

Whenever the term “at least”, “greater than”, or “greater than or equal to” precedes the first numerical value in a series of two or more numerical values, the term “at least”, “greater than,” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than”, “less than”, or “less than or equal to” precedes the first numerical value in a series of two or more numerical values, the term “no more than”, “less than”, or “less than or equal to” applies to each of the numerical values in that series of numerical values. For example, less than or equal to 3, 2, or 1 is equivalent to less than or equal to 3, less than or equal to 2, or less than or equal to 1.

The use of the term “including” as well as other forms, such as “include”, “includes,” and “included,” is not limiting.

The phrase “one or more pharmaceutically acceptable excipients” is used herein to refer that one pharmaceutically acceptable excipient or more than one pharmaceutically acceptable excipient may be used in any combination. The number of pharmaceutically acceptable excipients to be used may be at the discretion of a person skilled in the art, and they may be of different types.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “specific pH” herein refers to a desired pH value of a solvent or a solution comprising CocE obtained by adding a pharmaceutically acceptable excipient.

The term “% wt” is used to describe the weight percentage of one component in a mixture of components.

The term “a trace” herein refers more than, but close to about 0%.

The term “about” herein refers to ±10%, ±20%, ±30%, ±40%, or ±50%, or to the nearest significant figure.

An “amino acid” refers to a natural amino acid or a non-natural amino acid. The natural amino acid is a naturally-occurring amino acid, including, but not limited to, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, and tryptophan. The non-natural amino acid may be incorporated into peptides for many different reasons, for instance, to increase the activity or selectivity and plasma stability of peptides or to induce or stabilize secondary structure (e.g., helices, sheets, or turns). The non-natural amino acid includes, but not limited to, N-alkyl (e.g., N-methyl) amino acids, stereoisomers (D-amino acids), homo amino acids, alpha-methyl amino acids, beta¹ amino acids, beta² amino acids, beta³ amino acids, peptides, aminocyclohexanecarboxylate (ACHC), and non-protein amino acids, such as, citrulline, ornithine, homoalanine, norvaline, norleucine, etc. In some embodiment, the non-natural amino acid includes N-methyl alanine and citrulline.

As used herein, C₁-Cₓ includes C₁-C₂, C₁-C₃ . . . C₁-Cₓ. By way of example only, a group designated as “C₁-C₆” indicates that there are one to six carbon atoms in the moiety, e.g., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, e.g., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, e.g. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but not limited to, —CH₂—, —CH₂CH₂—, —CH₂CH₂CH₂—, —CH₂CH₂CH₂CH₂—, and the like. In some embodiments, an alkylene is —CH₂—.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

An “hydroxyalkyl” refers to an alkyl in which one hydrogen atom is replaced by a hydroxyl. In some embodiments, a hydroxyalkyl is a C₁-C₄hydroxyalkyl. Typical hydroxyalkyl groups include, but not limited to, —CH₂OH, —CH₂CH₂OH, —CH₂CH₂CH₂OH, —CH₂CH₂CH₂CH₂OH, and the like.

An “aminoalkyl” refers to an alkyl in which one hydrogen atom is replaced by an amino. In some embodiments, aminoalkyl is a C₁-C₄aminoalkyl. Typical aminoalkyl groups include, but not limited to, —CH₂NH₂, —CH₂CH₂NH₂, —CH₂CH₂CH₂NH₂, —CH₂CH₂CH₂CH₂NH₂, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (or vinyl), propenyl (or allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term, “heteroalkyl” generally refers to a straight-chain and/or branched hydrocarbon chain which has 1 to 30 carbon atoms and may be interrupted once or more than once by one or more of the groups —O—, —S—, —C(═O)—, —S(═O)—, —S(═O)₂—, —NR^{y}—, —NR^{y}C(═O)—, —C(═O)—NR^{y}—, —NR^{y}NR^{y}—, —S(═O)₂—NR^{y}NR^{y}—, —C(═O)—NR^{y}NR^{y}—, —CR^{x}═N—O—, and where the hydrocarbon chain including the side chains, if present, may be substituted by —NH—C(═O)—NH₂, —C(═O)—OH, —OH, —NH₂, —NH—C(═NNH₂)—, sulfonamide, sulfone, sulfoxide, sulfonic acid, sulfamide, or a combination thereof. In this context, R^{y} in each case is —H, phenyl, C₁-C₁₀-alkyl, C₂-C₁₀-alkenyl or C₂-C₁₀-alkynyl, which may in turn be substituted in each case by —NHC(O)NH₂, —COOH, —OH, —NH₂, —NH—C(═NNH₂)—, sulfonamide, sulfone, sulfoxide, sulfonic acid, sulfamide, or a combination thereof. In this context, R^{x} is —H, C₁-C₃-alkyl or phenyl. The terms “heteroalkyl-aryl” and “heteroalkyl-heteroaryl” as used herein generally refer to a heteroalkyl group (as defined above) substituted with an aromatic carbocycle or an aromatic heterocycle respectively; and wherein each is optionally substituted.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (or rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (or an arylene group).

As used herein, the term “aralkyl” generally refers to a monocyclic aromatic group (e.g., phenyl, naphthyl, etc.), to which a C₁₋₄-alkyl group is bonded. Illustrative aralkyl groups include benzyl and ethylphenyl.

The term “cycloalkyl” generally refers to a monocyclic or polycyclic aliphatic, non-aromatic radical, wherein each of the atoms forming the ring (or skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆cycloalkyl. In some embodiments, a cycloalkyl is a C₃-C₄cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, TH-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Monocyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl. The term “heteroaralkyl” as used herein generally refers to a lower alkyl group (e.g., C₁₋₆ alkyl) that is substituted with a heteraryl group. Illustrative examples include —CH₂-pyridine, —CH₂CH₂-triazole, etc.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

In some embodiments, each substituted alkyl, substituted fluoroalkyl, substituted heteroalkyl, substituted carbocycle, and substituted heterocycle is substituted with one or more R⁵ groups independently selected from the group consisting of halogen, C₁-C₆alkyl, monocyclic carbocycle, monocyclic heterocycle, —CN, —OR²¹, —CO₂R²¹, —C(═O)N(R²¹)₂, —N(R²¹)₂, —NR²¹C(═O)R²², —SR²¹, —S(═O)R²², —SO₂R²², or —SO₂N(R²¹)₂; each R²¹ is independently selected from hydrogen, C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl; or two R²¹ groups are taken together with the N atom to which they are attached to form a N-containing heterocycle; each R²² is independently selected from C₁-C₆alkyl, C₁-C₆fluoroalkyl, C₁-C₆heteroalkyl, C₃-C₆cycloalkyl, C₂-C₆heterocycloalkyl, phenyl, benzyl, 5-membered heteroaryl and 6-membered heteroaryl.

“Pharmaceutically acceptable,” as used herein, refers a material, such as a carrier or diluent, which does not abrogate the biological activity or properties of the compound, and is relatively nontoxic at the concentration or amount used, e.g., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Znrich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but not limited to, the interactions of an agonist, partial agonist, an inverse agonist, antagonist, degrader, or combinations thereof. In some embodiments, a modulator is an antagonist. In some embodiments, a modulator is an inhibitor.

The terms “administer,” “administering”, “administration,” and the like, as used herein, refer to the methods that may be used to enable delivery of compounds or compositions to the desired site of biological action. These methods include, but not limited to, oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a conjugate described herein, or a pharmaceutically acceptable salt thereof, and a co-agent, are both administered to a patient simultaneously in the form of a single entity or dosage. The term “non-fixed combination” means that the active ingredients, e.g. a conjugate described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating”, or “treatment,” as used herein, include alleviating, abating or ameliorating at least one symptom of a disease or condition, preventing additional symptoms, inhibiting the disease or condition, e.g., arresting the development or progression of the disease or condition, relieving the disease or condition, causing regression of the disease or condition, relieving a secondary condition caused by the disease or condition, or stopping the symptoms of the disease or condition either prophylactically or therapeutically.

The term “tumor microenvironment,” or “TME,” as used herein, includes the environment around a tumor, including the surrounding blood vessels, immune cells, fibroblasts, signaling molecules and the extracellular matrix. A tumor microenvironment includes tumor cells, necrotic cells, and cells surrounding the tumor which may be influenced (e.g., by signaling factors, growth factors, growth factor receptors, hormones, cytokines, and the like) by a tumor. A tumor microenvironment includes the area immediately surrounding a tumor (e.g., within about 1 cm of the tumor). In some instances, a tumor microenvironment includes cells, tissues, blood vessels, and other biological materials wherein the pH is lower than analogous tissues that are not within the vicinity or under the influence of a tumor. A tumor microenvironment may be defined by having a low (i.e., acidic) pH.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds disclosed herein, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

### EXAMPLES

As used above, and throughout the description of the invention, the following abbreviations, unless otherwise indicated, shall be understood to have the following meanings:

### ABBREVIATIONS

786-0 human tumour cell line
A431NS human tumour cell line
A549 human tumour cell line
ABCBl ATP-binding cassette sub-family B member I (synonym for P-gp and MDR1)
abs. absolute
Abu α(alpha)-amino butyric acid
Ac acetyl
ACN acetonitrile
ADC antibody drug conjugate
A2DC antibody 2 drug conjugate
aq. aqueous, aqueous solution
ATP adenosine triphosphate
BCRP breast cancer resistance protein, an efflux transporter
BEP 2-bromo-1-ethylpyridinium tetrafluoroborate
Boc tert-butoxycarbonyl
br. broad (in NMR)
Ex. Example
BxPC3 human tumour cell line
ca. circa, about
C-DAR cysteine drug to antibody ratio (linker attached to cystein residues)
Cl chemical ionization (in MS)
D doublet (in NMR)
D day(s)
TLC thin-layer chromatography
DCI direct chemical ionization (in MS)
DCM dichloromethane
Dd doublet of doublets (in NMR)
DIEA N, N diisopropyl ethyl amine (Hunig's base)
DMAP 4-N,N-dimethylaminopyridine
DME 1,2-dimethoxyethane
DMEM Dulbecco's modified eagle medium (standardized nutrient medium for cell culture)
DMF N,N-dimethylformamide
DMSO dimethyl sulphoxide
DAR drug to antibody ratio
DPBS Dulbecco's phosphate-buffered salt solution
Dt doublet of triplets (in NMR)
DTT DL-dithiothreitol
d. Th. of theory (in chemical yield)
EDC N′-(3-dimethylaminopropyl)-N-ethylcarbodi-imide hydrochloride
EGFR epidermal growth factor receptor
cc: enantiomeric excess
EI electron impact ionization (in MS)
ELISA enzyme-linked immunosorbent assay
eq. equivalent(s)
ESI electrospray ionization (in MS)
FCS foetal calf serum
Fmoc (9H-fluoren-9-ylmethoxy)carbonyl
sat. saturated
GTP guanosine-5′-triphosphate
h hour(s)
HATU O-(7-azabenzotriazol-1-yl)-N,N,N′,N′-tetramethyluronium hexafluoro phosphate
HCT-116 human tumour cell line
HEPES 4-(2-hydroxyethyl)piperazine-1-ethane-sulphonic acid
HOAc acetic acid
HOAt 1-hydroxy-7-azabenzotriazole
HOBt 1-hydroxy-1H-benzotriazole hydrate
HOSu N-hydroxysuccinimide
HPLC high-pressure, high-performance liquid chromatography
HT29 human tumour cell line
IC50 half-maximal inhibitory concentration
i.m. intramuscularly, administration into the muscle
i.v. intravenously, administration into the vein
K-DAR lysine drug to antibody ratio (linker attached to lysine residues)
KPL-4 human tumour cell lines
KU-19-19 human tumour cell line
LC-MS liquid chromatography-coupled mass spectrometry
LLC-PKl cells Lewis lung carcinoma pork kidney cell line
L-MDR human MDR1 transfected LLC-PKl cells
LoVo human tumour cell line
m multiplet (in NMR)
Me methyl
MDR1 Multidrug resistance protein I
MeCN acetonitrile
min minute(s)
MS mass spectrometry
MTBE methyl tert.-butyl ether
MTT 3-(4,5-dimethylthiazol-2-yl)-2,5-diphenyl-2H-tetrazolium bromide
NCI-H292 human tumour cell line
NCI-H520 human tumour cell line
NMM N-methylmorpholine
NMP N-methyl-2-pyrrolidinone
NMR nuclear magnetic resonance spectrometry
NMRI mouse strain originating from the Naval Medical Research Institute
Nude mice experimental animals
NSCLC non small cell lung cancer
PBS phosphate-buffered salt solution
Pd/C palladium on activated carbon
P-gp P-glycoprotein, a transporter protein
PNGaseF enzyme for cleaving sugar
Quant quantitative (in yield)
quart quartet (in NMR)
quint quintet (in NMR)
RT room temperature
Rt retention time (in HPLC)
s singlet (in NMR)
s.c. subcutaneously, administration under the skin
SCC-4 human tumour cell line
SCC-9 human tumour cell line
SCID mice test mice with severe combined immunodeficiency
SK-HEP-I human tumour cell line
t triplet (in NMR)
T3P® 2,4,6-tripropyl-1,3,5,2,4,6-trioxatriphosphi-nane 2,4,6-trioxide
TBAF tetra-n-butylammonium fluoride
TCEP tris(2-carboxyethyl)phosphine
TEMPO (2,2,6,6-tetramethylpiperidin-1-yl)oxyl
tert tertiary
TFA trifluoroacetic acid
THF tetrahydrofuran
TLC thin-layer chromatography
UV ultraviolet spectrometry
v/v volume to volume ratio (of a solution)
Z benzyloxycarbonyl

The following examples are provided for illustrative purposes only and not to limit the scope of the claims provided herein.

### Analytical Methods (LC-MS)

General method 1 (LC-MS): Instrument: Waters ACQUITY SQD UPLC System; Column: Waters Acquity UPLC HSS T3 1.8μ 50×1 mm; Eluent A: 1 l Water+0.25 mL 99% ige formic acid, Eluent B: 1 l acetonitrile+0.25 mL 99% formic acid; Gradient: 0.0 min 90% A→1.2 min 5% A→2.0 min 5% A Stove: 50° C.; Flow: 0.40 mL/min; UV-Detection: 208-400 nm.
Method 2 (LC-MS): System MS: Thermo Scientific FT-MS; System UHPLC+: Thermo Scientific UltiMate 3000; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm/Optimum Integration Path 210-300 nm
Method 3 (LC-MS): System MS: Waters TOF instrument; System UPLC: Waters Acquity I-CLASS; Column: Waters, HSST3, 2.1×50 mm, C18 1.8 μm; Eluent A: 1 l Water+0.01% Formic acid; Eluent B: 1 l Acetonitrile+0.01% Formic acid; Gradient: 0.0 min 2% B 0.5 min 2% B→7.5 min 95% B→10.0 min 95% B; Oven: 50° C.; Flow: 1.00 ml/min; UV-Detection: 210 nm
Method 4 (LC-MS): System MS: Thermo Scientific FT-MS; System UHPLC+: Thermo Scientific Vanquish; Column: Waters, HSST3, 2.1×75 mm, C18 1.8 μm; Eluent A: 1 l Water+0.01% Formic acid; Eluent B: 1 l Acetonitrile+0.01% Formic acid; Gradient: 0.0 min 10% B→2.5 min 95% B→3.5 min 95% B; Oven: 50° C.; Flow: 0.90 ml/min; UV-Detection: 210 nm
Method 5 (LC-MS): System MS: Waters TOF instrument; System UPLC: Waters Acquity I-CLASS; Column: Waters Acquity UPLC HSS T3 1.8 μm 50×1 mm; Eluent A: 1 l Water+0.100 ml 99% ige Formic acid, Eluent B: 1 l Acetonitrile+0.100 ml 99% ige Formic acid; Gradient: 0.0 min 90% A→1.2 min 5% A→2.0 min 5% A Oven: 50° C.; Flow: 0.40 ml/min; UV-Detection: 210 nm.
Method 6 (LC-MS): System MS: Waters TOF instrument; System UPLC: Waters Acquity I-CLASS; Column: Waters Acquity UPLC HSS T3 1.8 m 50×1 mm; Eluent A: 1l Water+0.100 ml 99% ige Formic acid, Eluent B: 1 l Acetonitrile+0.100 ml 99% ige Formic acid; Gradient: 0.0 min 95% A→6.0 min 5% A→7.5 min 5% A Oven: 50° C.; Flow: 0.35 ml/min; UV-Detection: 210 nm.

### Synthesis Examples

### Building Blocks

### Example S1: Preparation of (3R)-3-{[(4-aminophenyl)carbamoyl]amino}-3-{3-[({3-[(propyl carbamoyl)amino] phenyl} sulfonyl)amino]phenyl}propanoic acid (Building Block 1)

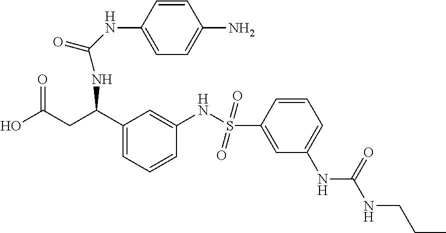 [SMILES: CCCNC(=O)Nc1cccc(S(=O)(=O)Nc2cccc([C@@H](CC(=O)O)NC(=O)Nc3ccc(N)cc3)c2)c1]

The synthesis of Building block 1 has been described in WO2020/094471

¹H-NMR (500 MHz, D₄-methanol): δ=0.93 (t, 3H), 1.5 (m, 2H), 2.74 (d, 2H), 3.1 (dt, 2H), 5.15 (t, 1H), 6.68 (d, 2H), 6.85 (d, 1H), 7.05 (d, 1H), 7.1 (d, 1H), 7.13 (t, 1H), 7.28-7.4 (m, 3H), 7.6 (s, 1H), 7.66 (d, 1H).

### Example S2: Preparation of (3R)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Building Block 2)

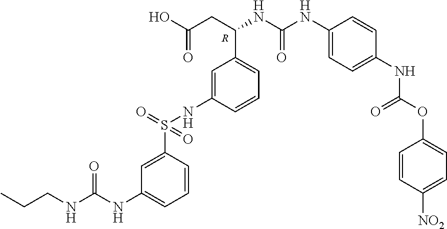

The synthesis of Building block 2 has been described in WO2020/094471.

### Example S3: Preparation of (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building Block 3)

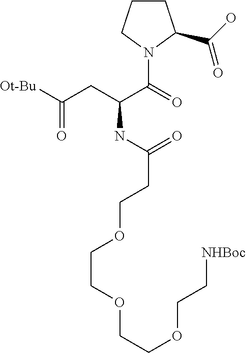 [SMILES: CC(C)(C)OC(=O)C[C@H](NC(=O)CCOCCOCCOCCNC(=O)OC(C)(C)C)C(=O)N1CCC[C@H]1C(=O)O]

Building block 3 was synthesized using classical methods of peptide synthesis starting with the coupling of Z-Asp(OtBu)-OH with benzyl L-prolinate hydrochloride (1:1) in THF in the presence of T3P and DIPEA and subsequent removal of the Z-protecting group as well as the benzyl ester by hydrogenolysis over Pd/C to give (2S)-1-[(2S)-2-amino-4-tert-butoxy-4-oxobutanoyl] pyrrolidine-2-carboxylic acid. This partially protected dipeptide was acylated with tert-butyl{2-[2-(2-{3-[(2,5-dioxopyrrolidin-1-yl)oxy]-3-oxopropoxy}ethoxy)ethoxy]ethyl} carbamate to give the title compound. Tert-butyl{2-[2-(2-{3-[(2,5-dioxopyrrolidin-1-yl)oxy]-3-oxopropoxy}ethoxy) ethoxy]ethyl}carbamate was previously prepared by reacting 2,2-dimethyl-4-oxo-3,8,11,14-tetraoxa-5-azaheptadecan-17-oic acid with N-Hydroxysuccinimide in dioxane in the presence of EDCI. LC-MS: Rₜ=0.81 min; MS (ESIpos): m/z=590 [M+H]⁺.

### Example S4: Preparation of N-{2-[{2-[(tert-butoxycarbonyl)(methyl)amino]ethyl}(methyl)amino]ethyl}-N-methylglycine (Building Block 4)

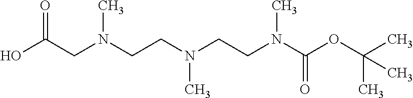 [SMILES: CN(CCN(C)CC(=O)O)CCN(C)C(=O)OC(C)(C)C]

Initially, tert-butyl methyl(2-{methyl[2-(methylamino)ethyl] amino}ethyl)carbamate was obtained starting from commercially available N,N′-dimethyl-N-[2-(methylamino)ethyl]ethane-1,2-diamine.

Benzyl bromoacetate (609 mg, 2.66 mmol) was initially dissolved under argon in acetonitrile (25 mL) and potassium carbonate (734 mg, 5.31 mmol) was added. The solution was cooled to 0° C. and a solution of tert-butyl methyl(2-{methyl[2-(methylamino)ethyl]amino}ethyl)carbamate (652 mg, 2.66 mmol) in acetonitrile was added. The batch was stirred at rt for 1 h and subsequently filtered. The filtrate was evaporated in vacuo and the remaining residue was purified by prep. HPLC. Relevant fractions were collected and evaporated to dryness. 665 mg (97% purity, 62% yield) of the protected intermediate benzyl N-{2-[{2-[(tert-butoxycarbonyl)(methyl)amino]ethyl}(methyl)amino]ethyl}-N-methylglycinate were obtained as a colorless oil. This intermediate (687 mg, 1.75 mmol) was dissolved in DCM/methanol and hydrogenated over 10% Pd on charcoal at rt for 2 h. The catalyst was filtered off and the filtrate was concentrated in vacuo to give Building block 4 (555 mg, 61% purity, 64% yield) as a colorless oil. LC-MS: Rₜ=0.74 min; MS (ESIpos): m/z=304 [M+H]⁺

### Example S5: Preparation of N-{2-[{2-[(tert-butoxycarbonyl)(methyl)amino]ethyl}(methyl)amino]ethyl}-N-methylglycyl-L-asparaginyl-L-proline (Building Block 5)

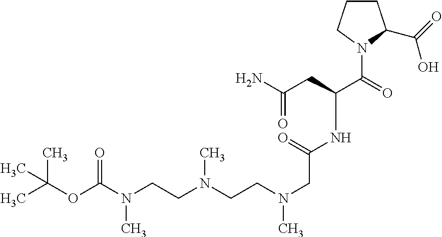 [SMILES: CN(CCN(C)CC(=O)N[C@@H](CC(N)=O)C(=O)N1CCC[C@H]1C(=O)O)CCN(C)C(=O)OC(C)(C)C]

Building block 5 was synthesized using classical peptide synthesis methods starting with the coupling of Boc-Asn with benzyl L-prolinate hydrochloride (1:1) in DMF in the presence of HATU and N,N-Diisopropylethylamine and subsequent removal of the Boc-protecting group with TFA in DCM. This partially protected dipeptide was acylated with N-{2-[{2-[(tert-butoxycarbonyl)(methyl)amino]ethyl}(methyl)amino]ethyl}-N-methylglycine (Building Block 4) in DMF in the presence of HATU and N, N-Diisopropylethylamine. In the final step the benzylester was removed by hydrogenolysis over 10% Pd/charcoal. LC-MS: Rt=0.81 min; MS (ESIpos): m/z=515 [M+H]⁺

### Example S6: Preparation of N-(tert-butoxycarbonyl)-L-alanyl-N-methyl-L-alanine (Building Block 6)

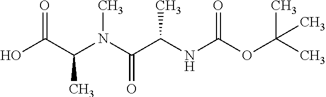 [SMILES: C[C@H](NC(=O)OC(C)(C)C)C(=O)N(C)[C@@H](C)C(=O)O]

Building block 6 was synthesized by coupling 2,5-dioxopyrrolidin-1-yl N-(tert-butoxycarbonyl)-L-alaninate to N-methyl-L-alanine in DMF in the presence of N,N-diisopropylethylamine.

### Example S7: Preparation of N-methyl-N-(2,2,8,11-tetramethyl-4-oxo-3-oxa-5,8,11-triazatridecan-13-yl)glycine (Building Block 7)

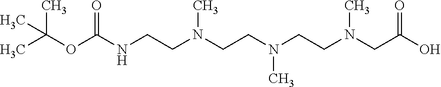 [SMILES: CN(CCNC(=O)OC(C)(C)C)CCN(C)CCN(C)CC(=O)O]

Commercially available N,N′-dimethyl-N-[2-(methylamino) ethyl]ethane-1,2-diamine (500 mg, 3.44 mmol) was dissolved under argon in acetonitrile (50 mL) and potassium carbonate (476 mg, 3.44 mmol) was added. The solution was cooled to 0° C. and a solution of benzyl bromoacetate (394 mg, 1.72 mmol)) in acetonitrile was added. The batch was stirred at rt for 20 h and was filtered subsequently. The filtrate was evaporated and the remaining residue was purified by prep. HPLC. Relevant fractions were collected and evaporated to dryness to yield 476 mg (99% purity, 34% yield) of the protected intermediate as a colorless oil. This intermediate was alkylated reductively in methanol and 2.5 equivalents of acetic acid with tert-butyl (2-oxoethyl)carbamate in the presence of trihydrido(pyridine)boron. Subsequently, the benzylester was removed by hydrogenolysis over 10% Pd/charcoal. LC-MS: Rₜ=0.47 min; MS (ESIpos): m/z=347 [M+H]⁺. ¹H NMR (600 MHz, DMSO-d₆) δ ppm 1.394 (s, 9H) 2.378 (s, 3H) 2.542 (s, 2H) 2.779 (s, 3H) 2.812 (s, 3H) 2.852-2.944 (m, 4H) 3.145 (br t, J=6.26 Hz, 2H) 3.229 (br t, J=5.87 Hz, 1H) 3.262-3.344 (m, 4H) 3.975 (s, 3H) 7.098 (br t, J=5.09 Hz, 1H).

### Example S8: Preparation of N-methyl-N-(2,2,8,11-tetramethyl-4-oxo-3-oxa-5,8,11-triazatridecan-13-yl)glycyl-L-asparaginyl-L-proline (Building Block 8)

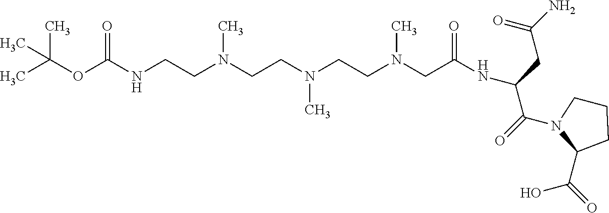 [SMILES: CN(CCNC(=O)OC(C)(C)C)CCN(C)CCN(C)CC(=O)N[C@@H](CC(N)=O)C(=O)N1CCC[C@H]1C(=O)O]

Building block 8 was synthesized using classical peptide synthesis methods starting with the coupling of Boc-Asn with benzyl L-prolinate hydrochloride (1:1) in DMF in the presence of HATU and N, N-diisopropylethylamine and subsequent removal of the Boc-protecting group with TFA in DCM to yield the dipeptide H-L-Asn-L-Pro-OBzl as the trifluoroacetate salt. This partially protected dipeptide was acylated with N-methyl-N-(2,2,8,11-tetramethyl-4-oxo-3-oxa-5,8,11-triazatridecan-13-yl)glycine (Building Block 7) in DMF in the presence of HATU and N, N-diisopropylethylamine. In the final step, the benzylester was removed by hydrogenolysis over 10% Pd/charcoal. LC-MS: Rₜ=0.54 min; MS (ESIpos): m/z=558 [M+H]⁺. ¹H NMR (600 MHz, DMSO-d₆) δ ppm 1.393 (s) 1.851-1.956 (m) 2.074 (s) 2.129 (br dd, J=12.13, 8.22 Hz) 2.365-2.413 (m) 2.480-2.516 (m) 2.543 (s) 2.768 (s) 2.820 (s) 2.843-2.906 (m) 3.158 (br t, J=6.06 Hz) 3.217 (br t, J=5.87 Hz) 3.284-3.347 (m) 3.383 (dd, J=8.51, 5.77 Hz) 3.613-3.654 (m) 3.793 (br d, J=13.30 Hz) 4.227 (dd, J=8.61, 4.11 Hz) 4.712-4.752 (m) 4.848-4.902 (m) 4.915-4.955 (m) 6.902 (br s) 6.966 (s) 7.103 (br t, J=5.28 Hz) 7.452 (br s) 7.472 (br s) 8.769-8.817 (m) 8.941 (br d, J=7.63 Hz).

### Example S9: Preparation of (4S)-4,11-diethyl-3,14-dioxo-3,4,12,14-tetrahydro-1H-pyrano[3′,4′:6,7] indolizino[1,2-b]quinolin-4-yl L-valinate·trifluoroacetic acid (1:1) (Building block 9)

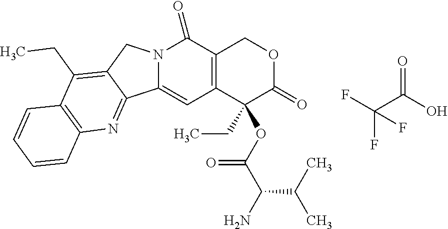

2.59 g (10.6 mmol) of N-(tert-butoxycarbonyl)-valine-N-carboxyanhydride and 0.5 g of 4-(N,N-dimethylamino)-pyridine were added to a stirred suspension of 2 g (5.3 mmol) of (4S)-4,11-diethyl-4-hydroxy-1H-pyrano[3′,4′:6,7]indolizino[1,2-b]quinoline-3,14(4H,12H)-dione in 150 ml of absolute dichloromethane. The mixture was stirred at rt for 20 h and subsequently concentrated in vacuo. 8 ml ACN were added to the residue and subsequently 5 mL diethyl ether. The mixture was filtrated and the remaining residue was dried in vacuo. 2964 mg (92%) of the protected intermediate were obtained. LC-MS: Rₜ=1.19 min; MS (ESIpos): m/z=576 (M+H)⁺. Next, 2964 mg (5.15 mmol) of this Boc-protected intermediate compound in 6 ml of dichloromethane and 60 ml of anhydrous trifluoroacetic acid was stirred for 30 min. at rt and subsequently sonicated for 1 h. After concentrating in vacuo the product was lyophilized from a mixture of acetonitrile/water. 3.622 g (quant) of Building block 9 obtained. LC-MS: Rt=0.68 min; MS (ESIpos): m/z=476 [M+H]⁺.

### Example S10: Preparation of (3S)-3-{[(4-aminophenyl)carbamoyl]amino}-3-{3-[({3-[(propyl carbamoyl)amino] phenyl} sulfonyl)amino]phenyl}propanoic acid (Building Block 10)

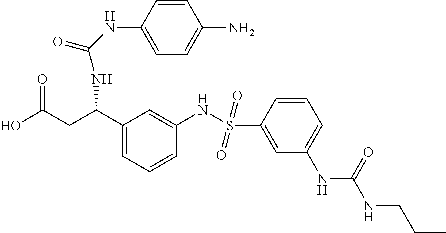 [SMILES: CCCNC(=O)Nc1cccc(S(=O)(=O)Nc2cccc([C@H](CC(=O)O)NC(=O)Nc3ccc(N)cc3)c2)c1]

The synthesis of Building block 10 has been described in WO2020/094471.

### Example S11: Preparation of (3S)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Building Block 11)

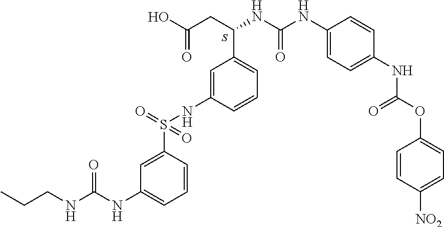

The synthesis of Building block 11 has been described in WO2020/094471. LC-MS: Rₜ=0.89 min; MS (ESIpos): m/z=720 [M+H]⁺.

### Intermediates

### Example S12: Preparation of (4S)-4,11-diethyl-3,14-dioxo-3,4,12,14-tetrahydro-1H-pyrano[3′,4′:6,7]indolizino[1,2-b]quinolin-4-yl glycyl-L-prolyl-L-valinate·trifluoroacetic acid (1:1) (Intermediate 1)

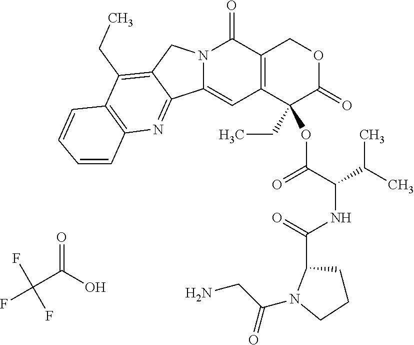 [SMILES: O=C(O)C(F)(F)F ; CCc1c2c(nc3ccccc13)-c1cc3c(c(=O)n1C2)COC(=O)[C@@]3(CC)OC(=O)[C@@H](NC(=O)[C@@H]1CCCN1C(=O)CN)C(C)C]

Step 1: Trifluoroacetic acid-(4S)-4,11-diethyl-3,14-dioxo-3,4,12,14-tetrahydro-1H-pyrano[3′,4′:6,7] indolizino[1,2-b]quinolin-4-yl L-valinate (1:1) (150 mg, 254 μmol) (Building block 9) (150 mg, 254 μmol) and N-(tert-butoxycarbonyl)glycyl-L-proline (83.1 mg, 305 μmol) were dissolved in DMF (10 mL) and 1.3 eq EDCI (63.4 mg, 331 μmol), 1.5 eq HOBT hydrate (58.4 mg, 382 μmol) as well as 3 eq N,N-diisopropylethylamine (130 μl, 760 μmol) were added. After stirring for 1 h at rt, the mixture was concentrated in vacuo and the residual was purified by prep. HPLC, then concentrated and lyophilized to yield Boc-Intermediate 1 (105 mg, 100% purity, 49%). LC-MS: Rₜ=3.39 min; MS (ESIpos): m/z=730 [M+H]⁺.
Step 2: (4S)-4,11-diethyl-3,14-dioxo-3,4,12,14-tetrahydro-1H-pyrano[3′,4′:6,7]indolizino[1,2-b]quinoline-4-yl N-(tert-butoxycarbonyl)glycyl-L-prolyl-L-valinate (Boc-Intermediate 1) (105 mg, 144 μmol) was dissolved in DCM (10 mL), TFA (2 mL) was added and the reaction mixture was stirred for 30 min. It was concentrated in vacuo, dissolved in ACN/H₂O and lyophilized to give the Intermediate 1 in quantitative yield (101 mg, 95% purity, 90% yield). LC-MS: Rₜ=2.03 min; MS (ESIpos): m/z=630 [M+H]⁺.

### Example S13: Preparation of (4S)-4,11-diethyl-3,14-dioxo-3,4,12,14-tetrahydro-1H-pyrano[3′,4′:6,7] indolizino[1,2-b]quinolin-4-yl L-asparaginyl-L-prolyl-L-valinate·trifluoroacetic acid (1:1) (Intermediate 2)

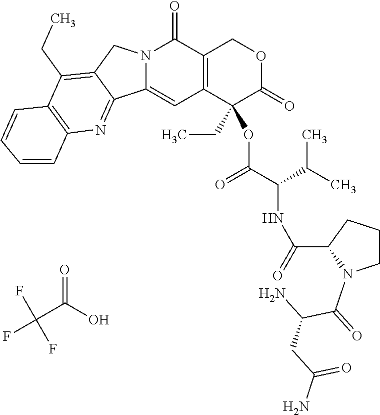 [SMILES: CCc1c2c(nc3ccccc13)-c1cc3c(c(=O)n1C2)COC(=O)[C@@]3(CC)OC(=O)[C@@H](NC(=O)[C@@H]1CCCN1C(=O)[C@@H](N)CC(N)=O)C(C)C ; O=C(O)C(F)(F)F]

Step 1: Trifluoroacetic acid-(4S)-4,11-diethyl-3,14-dioxo-3,4,12,14-tetrahydro-1H-pyrano[3′,4′:6,7] indolizino[1,2-b]quinolin-4-yl L-valinate (1/1) (150 mg, 254 μmol) (Building block 9) (150 mg, 254 μmol) and N²-(tert-butoxycarbonyl)-L-asparaginyl-L-proline (101 mg, 305 μmol) were dissolved in DMF (10 mL) and 1.3 eq EDCI (63.4 mg, 331 μmol), 1.5 eq HOBT hydrate (58.4 mg, 382 μmol) as well as 3 eq N,N-diisopropylethylamine (130 μl, 760 μmol) were added. After stirring for 1 h at rt, the mixture was concentrated in vacuo. The residual was purified by prep. HPLC, then concentrated and lyophilized to yield Boc-Intermediate 2 (185 mg, 93% purity, 86%). LC-MS: Rₜ=3.08 min; MS (ESIpos): m/z=787 [M+H]⁺.
Step 2: (4S)-4,11-diethyl-3,14-dioxo-3,4,12,14-tetrahydro-1H-pyrano[3′,4′:6,7]indolizino[1,2-b]quinolin-4-yl N²-(tert-butoxycarbonyl)-L-asparaginyl-L-prolyl-L-valinate (185 mg, 235 μmol) was dissolved in DCM (15 mL), TFA (3 mL) was added and the reaction mixture was stirred for 30 min and concentrated in vacuo. The residual was dissolved in ACN/water and lyophilized to yield Intermediate 2 (188 mg, 100% purity, quant.). LC-MS: Rₜ=1.13 min; MS (ESIpos): m/z=687 [M+H]⁺

### Example S14: Preparation of (4S)-4,11-diethyl-3,14-dioxo-3,4,12,14-tetrahydro-1H-pyrano[3′,4′:6,7] indolizino[1,2-b]quinolin-4-yl N-methyl-N-(2-{methyl[2-(methylamino) ethyl]amino}ethyl)glycyl-L-asparaginyl-L-prolyl-L-valinate·trifluoroacetic acid (1:1) (Intermediate 3)

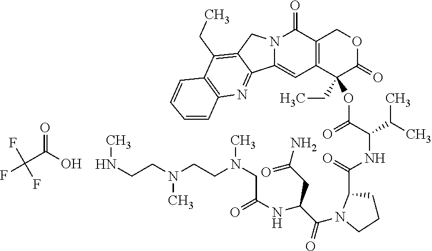 [SMILES: O=C(O)C(F)(F)F ; CCc1c2c(nc3ccccc13)-c1cc3c(c(=O)n1C2)COC(=O)[C@@]3(CC)OC(=O)[C@@H](NC(=O)[C@@H]1CCCN1C(=O)[C@H](CC(N)=O)NC(=O)CN(C)CCN(C)CCNC)C(C)C]

Step 1: Trifluoroacetic acid-(4S)-4,11-diethyl-3,14-dioxo-3,4,12,14-tetrahydro-1H-pyrano[3′,4′:6,7] indolizino[1,2-b]quinolin-4-yl L-asparaginyl-L-prolyl-L-valinate (1/1) (Intermediate 2) (140 mg, 175 μmol) and N-{2-[{2-[(tert-butoxycarbonyl)(methyl)amino]ethyl}(methyl)amino]ethyl}-N-methylglycine (63.7 mg, 0.21 mmol) (building block 4) were dissolved in DMF (10 mL), and 1.5 eq HATU (99.7 mg, 262 μmol) and 3.0 eq N,N-diisopropylethylamine (91 μl, 520 μmol) were added. After stirring for 1 h at rt, the mixture was concentrated in vacuo and the residual was purified by prep. HPLC, then concentrated in vacuo to yield Boc-Intermediate 3 as a light yellow foam (138 mg, 100% purity, 81%). LC-MS: Rₜ=1.45 min; MS (ESIpos): m/z=972 [M+H]⁺
Step 2: (4S)-4,11-w (Boc Intermediate 3) (138 mg, 142 μmol) was dissolved in DCM (0.5 mL), TFA (5 mL) was added, and the reaction mixture was stirred for 1 h. It was concentrated in vacuo, dissolved in ACN/H₂O and lyophilized to give Intermediate 3 in quantitative yield (143 mg, 100% purity, quant.). LC-MS: Rₜ=1.03 min; MS (ESIneg): m/z=870 [M−H].

### Example S15: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-aminoethyl)(methyl) amino]ethyl}(methyl)amino]ethyl}-N-methylglycyl-L-asparaginyl-L-prolyl-L-valinate·trifluoroacetic acid (1:1) (Intermediate 4)

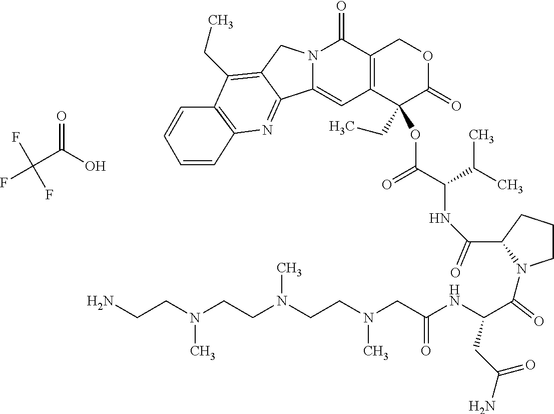

Step 1: Trifluoroacetic acid-(4S)-4,11-diethyl-3,14-dioxo-3,4,12,14-tetrahydro-1H-pyrano[3′,4′:6,7] indolizino[1,2-b]quinolin-4-yl L-asparaginyl-L-prolyl-L-valinate (1/1) (Intermediate 2) (150 mg, 187 μmol) was dissolved in DMF (12 mL) and 1.5 eq HATU (107 mg, 281 μmol), 1 eq N-methyl-N-(2,2,8,11-tetramethyl-4-oxo-3-oxa-5,8,11-triazatridecan-13-yl)glycine (Building Block 7) (64.9 mg, 187 μmol) as well as 3.0 eq N,N-diisopropylethylamine (98 μl, 560 μmol) were added. After stirring for 1 h at rt, the mixture was concentrated in vacuo and the residual was purified by prep. HPLC, then concentrated in vacuo to yield Boc-Intermediate 4 as a light yellow foam (128 mg, 55% purity, 37%). LC-MS: Rₜ=2.83 min; MS (ESIpos): m/z=1015 [M+H]⁺.
Step 2: (4S)-4,11-diethyl-3,14-dioxo-3,4,12,14-tetrahydro-1H-pyrano[3′,4′:6,7]indolizino[1,2-b]quinolin-4-yl N-methyl-N-(2,2,8,11-tetramethyl-4-oxo-3-oxa-5,8,11-triazatridecan-13-yl) glycyl-L-asparaginyl-L-prolyl-L-valinate (128 mg, 55% purity, 69.3 μmol) was dissolved in DCM (15 mL), TFA (3 mL) was added and the reaction mixture was stirred for 30 min. It was concentrated in vacuo, and the residual was purified by prep. HPLC, then concentrated in vacuo to yield Intermediate 4 as a yellow foam (88 mg, 100% purity, quant.). LC-MS: Rₜ=2.46 min; MS (ESIpos): m/z=915 [M+H]⁺.

### Example S16: Preparation of (4S)-4,11-diethyl-3,14-dioxo-3,4,12,14-tetrahydro-1H-pyrano[3′,4′:6,7] indolizino[1,2-b]quinolin-4-yl N-{2-[{2-[(2-aminoethyl)(methyl)amino]ethyl}(methyl)amino]ethyl}-N-methylglycylglycyl-L-prolyl-L-valinate·trifluoroacetic acid (1:1) (Intermediate 5)

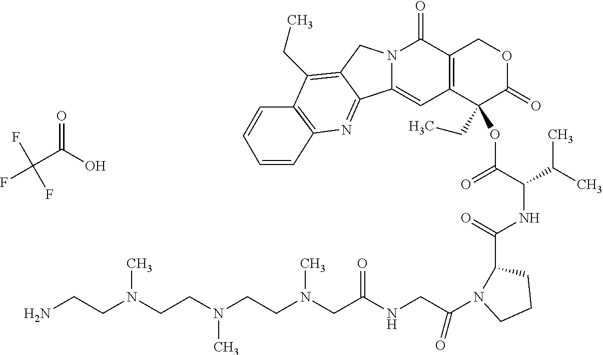

Step 1: Trifluoroacetic acid-(4S)-4,11-diethyl-3,14-dioxo-3,4,12,14-tetrahydro-1H-pyrano[3′,4′:6,7] indolizino[1,2-b]quinolin-4-yl glycyl-L-prolyl-L-valinate (1/1) (Intermediate 1) (101 mg, 136 μmol) were dissolved in DMF (10 mL) and 1.5 eq HATU (77.5 mg, 204 μmol), 1 eq N-methyl-N-(2,2,8,11-tetramethyl-4-oxo-3-oxa-5,8,11-triazatridecan-13-yl)glycine (Building Block 7) (56.5 mg, 163 μmol) as well as 3.0 eq N,N-diisopropylethylamine (71 μl, 410 μmol) were added. After stirring for 1 h at rt, the mixture was concentrated in vacuo and the residual was purified by prep. HPLC, then concentrated in vacuo to yield Boc-Intermediate 5 as a light yellow foam (90.6 mg, 98% purity, 69%). LC-MS: Rₜ=2.09 min; MS (ESIpos): m/z=958 [M+H]⁺.
Step 2: (4S)-4,11-diethyl-3,14-dioxo-3,4,12,14-tetrahydro-1H-pyrano[3′,4′:6,7]indolizino[1,2-b]quinolin-4-yl N-methyl-N-(2,2,8,11-tetramethyl-4-oxo-3-oxa-5,8,11-triazatridecan-13-yl) glycylglycyl-L-prolyl-L-valinate (90.6 mg, 94.6 μmol) were dissolved in DCM (6 mL), TFA (1.2 mL) was added and the reaction mixture was stirred for 30 min. It was concentrated in vacuo, the residue was dissolved in ACN/H₂O and lyophilized to give Intermediate 5 as a colorless foam (94 mg, 100% purity, quant.). LC-MS: Rₜ=0.99 min; MS (ESIneg): m/z=856 [M−H]⁻.

### Example S17: Preparation of (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Boc-Intermediate 6)

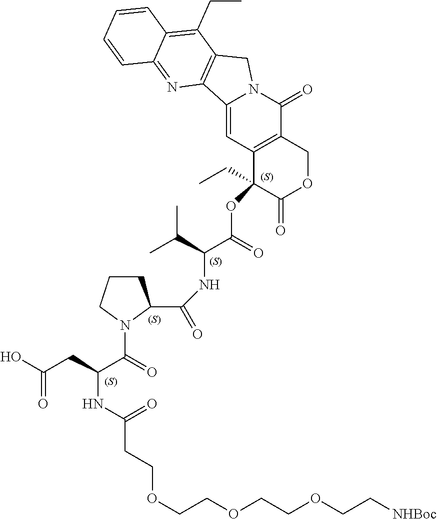

Step 1: (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (Building Block 3) (50.0 g, 84.8 mmol) was dissolved in 900 ml DMF and the solution was cooled down to 0° C. 1.1 eq 1-(3-Dimethylaminopropyl)-3-ethylcarbodiimidhydrochloride (17.0 g, 88.8 mmol) and 1.3 eq Ethyl cyanohydroxyiminoacetate (14.9 g, 105 mmol) and trifluoroacetic acid-(4S)-4,11-diethyl-3,14-dioxo-3,4,12,14-tetrahydro-1H-pyrano[3′,4′:6,7] indolizino[1,2-b]quinolin-4-yl L-valinate (1/1) (Building Block 9) (47.6 g, 80.8 mmol) were added and subsequently 3.0 eq (42 ml, 240 mmol) N,N-diisopropylethylamine was added dropwise. The mixture was stirred overnight at 0° C. It was diluted with 4 L EtOAc and the organic layer was washed with 10% aqueous citric acid (2×2 l), with 10% aqueous NaHCO₃-solution (2×2 l) and with saturated aqueous NaCl-solution (2×3 l). Subsequently it was dried over Mg₂SO₄, filtered and concentrated. The residue was dissolved in 250 ml DCM and purified using flash chromatography (DCM:MeOH 100:1). Relevant fractions were collected and evaporated in vacuo to yield OtBu-Intermediate 6 as a yellow foam (72.8 g, 98% purity, 84% yield).
Step 2: 12 g (11.5 mmol) of tert-butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl} pyrrolidine-1-carbonyl]-2,2-dimethyl-4,17-dioxo-3,8,11,14-tetraoxa-5,18-diazahenicosan-21-oate (OtBu-Intermediate 6) was dissolved in 100 ml of dichloromethane and 40 ml of anhydrous trifluoroacetic acid was added and the solution was stirred for 2 days at rt. After concentrating in vacuo 50 ml toluene were added and again evaporated. The residue was dissolved in 50 ml DCM/MeOH and subsequently poured into 600 ml of diethyl ether. The precipitating product (Intermediate 6) was filtered, washed with diethyl ether and dried in vacuo. (11.7 g, 95% purity, 97% yield).
Step 3: 1 g (995 μmol) of this intermediate (4S)-4,11-diethyl-3,14-dioxo-3,4,12,14-tetrahydro-1H pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate·trifluoroacetic acid (1:1) (Intermediate 6) was dissolved in 11 ml DMF and 1-[(tert-butoxycarbonyl)oxy]pyrrolidine-2,5-dione (214 mg, 995 μmol) and 1 eq N,N-diisopropylethylamine (170 μl, 1000 μmol) were added. The batch was stirred for 20 h at rt. Another ½ eq of BOC-OSu and N,N-diisopropylethylamine were added and the mixture was stirred for further 3 h at rt. Then the batch was evaporated to dryness using a rotary evaporator. The residue was separated by HPLC. Relevant fractions were collected and evaporated in vacuo to yield 744 mg (100% purity, 75% yield) of Boc-Intermediate 6 as a yellow foam. LC-MS: Rt=4.19 min; MS (ESIpos): m/z=991 [M+H]⁺.

### Example S18: Preparation of (14S)-1-Amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl} pyrrolidine-1-carbonyl]-N,N,N-trimethyl-12,16-dioxo-3,6,9,17-tetraoxa-13-azanonadecan-19-aminium·trifluoroacetate·trifluoroacetic acid (1:1:1) (Intermediate 7)

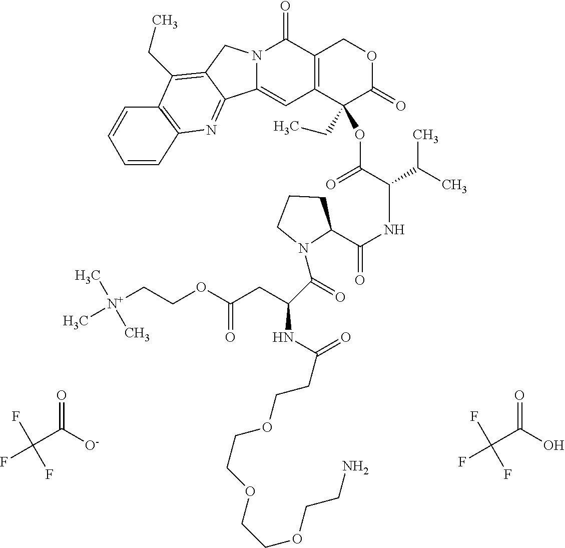

Step 1: (4S)-4,11-Diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Boc-Intermediate 6) (50.0 mg, 50.4 μmol) was dissolved in 10 mL dichloromethane and 1.2 eq 2-hydroxy-N,N,N-trimethylethan-1-aminium chloride (8.45 mg, 60.5 μmol) as well as 1.2 eq sodium hydrogen carbonate (5.09 mg, 60.5 μmol), 1.3 eq 1-(3-dimethylaminopropyl)-3-ethylcarbodiimide hydrochloride (12.6 mg, 65.6 μmol) and 5 mg (40.9 μmol) 4-(dimethylamino)pyridine were added. After stirring for 20 h at rt, the same amounts of 2-hydroxy-N,N,N-trimethylethan-1-aminium chloride, sodium hydrogen carbonate, 1-(3-dimethylaminopropyl)-3-ethylcarbodiimide hydrochloride and 4-(dimethylamino)pyridine were added and the mixture was stirred for another 4 h at rt. Then the mixture was concentrated in vacuo and the residual was purified by prep. HPLC. Relevant fractions were collected and concentrated in vacuo to yield Boc-Intermediate 7 as a yellow foam (21 mg, 95% purity, 33% yield). LC-MS: Rₜ=2.59 min; MS (ESIpos): m/z=1076 [M+H]⁺.
Step 2: (19S)-19-[(2S)-2-{[(2S)-1-{[(4S)-4,11-Diethyl-3,14-dioxo-3,4,12,14-tetrahydro-1H-pyrano [3′,4′:6,7]indolizino[1,2-b]quinolin-4-yl]oxy}-3-methyl-1-oxobutan-2-yl]carbamoyl}pyrrolidine-1-carbonyl]-N,N,N,2,2-pentamethyl-4,17,21-trioxo-3,8,11,14,22-pentaoxa-5,18-diazatetracosan-24-aminium trifluoroacetate (Boc-Intermediate 7) (20.0 mg, 16.8 μmol) were dissolved in 5 ml DCM and 1 ml TFA was added and the reaction mixture was stirred for 30 min. It was concentrated in vacuo, the residue was dissolved in ACN/H₂O and lyophilized to give Intermediate 7 (15 mg, 100% purity, 74% yield). LC-MS: Rₜ=1.79 min; MS (ESIpos): m/z=976 [M+H]F.

### Example S19: Preparation of 4-(dimethylamino)butyl (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl] oxy}-3-methyl-1-oxobutan-2-yl]carbamoyl}pyrrolidine-1-carbonyl]-12-oxo-3,6,9-trioxa-13-azahexadecan-16-oate·trifluoroacetic acid (1:1) (Intermediate 8)

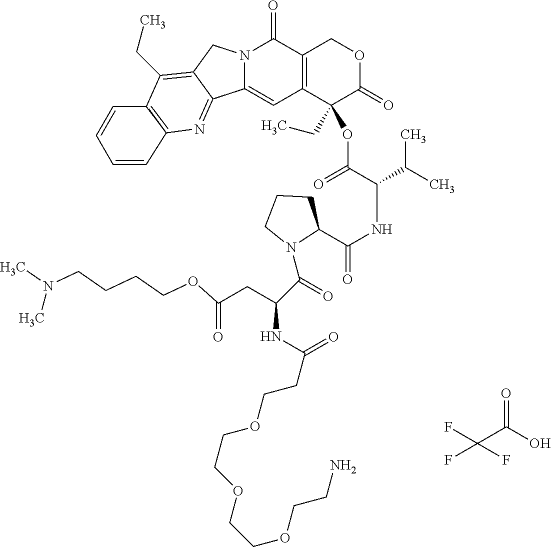

Step 1: (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Boc-Intermediate 6) (50.0 mg, 50.4 μmol) was dissolved in 10 mL DMF and 10 eq 4-(dimethylamino)butan-1-ol (59 mg, 504 μmol) as well as 4 eq 1-(3-dimethylaminopropyl)-3-ethylcarbodiimide hydrochloride (38.7 mg, 202 μmol) and 25 mg (202 μmol) 4-(dimethylamino)pyridine were added. After stirring for 20 h at rt the mixture was concentrated in vacuo and the residual was purified by preparative HPLC. Relevant fractions were collected and concentrated in vacuo to yield Boc-Intermediate 8 as a yellow foam (33.5 mg, 95% purity, 58% yield). LC-MS: Rₜ=2.66 min; MS (ESIpos): m/z=1090 [M+H]⁺.
Step 2: 4-(Dimethylamino)butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl] carbamoyl}pyrrolidine-1-carbonyl]-2,2-dimethyl-4,17-dioxo-3,8,11,14-tetraoxa-5,18-diazahenicosan-21-oate (Boc-Intermediate 8) (33.5 mg, 95% purity, 29.3 μmol) was dissolved in 6 ml DCM and 1 ml TFA was added and the reaction mixture was stirred for 30 min. It was concentrated in vacuo, the residue was dissolved in ACN/H₂O and lyophilized to give Intermediate 8 (34 mg, 98% purity, quant.). LC-MS: Rₜ=1.75 min; MS (ESIpos): m/z=990 [M+H]⁺.

### Example S20: Preparation of 3-(dimethylamino)propyl (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl] oxy}-3-methyl-1-oxobutan-2-yl]carbamoyl}pyrrolidine-1-carbonyl]-12-oxo-3,6,9-trioxa-13-azahexadecan-16-oate·trifluoroacetic acid (1:1) (Intermediate 9)

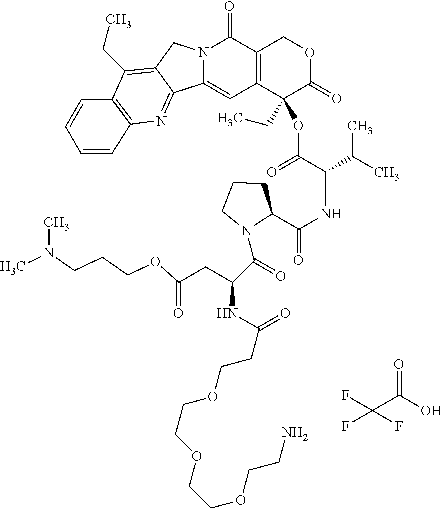

Step 1: (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Boc-Intermediate 6) (50.0 mg, 50.4 μmol) was dissolved in 10 mL DMF and 10 eq 3-(dimethylamino)propan-1-ol (52 mg, 504 μmol) as well as 4 eq 1-(3-dimethylaminopropyl)-3-ethylcarbodiimide hydrochloride (38.7 mg, 202 μmol) and 25 mg (202 μmol) 4-(dimethylamino)pyridine were added. After stirring for 20 h at rt the mixture was concentrated in vacuo and the residual was purified by prep. HPLC. Relevant fractions were collected and concentrated in vacuo, dissolved in ACN/water and lyophilized to yield Boc-Intermediate 9 as a yellow foam (34 mg, 98% purity, 61% yield). LC-MS: Rₜ=2.66 min; MS (ESIpos): m/z=1076 [M+H]⁺.
Step 2: 3-(dimethylamino)propyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl] carbamoyl}pyrrolidine-1-carbonyl]-2,2-dimethyl-4,17-dioxo-3,8,11,14-tetraoxa-5,18-diazahenicosan-21-oate (Boc-Intermediate 9) (34 mg, 98% purity, 30.8 μmol) was dissolved in 6 ml DCM and 1 ml TFA was added and the reaction mixture was stirred for 30 min. It was concentrated in vacuo, the residue was dissolved in ACN/H₂O and lyophilized to give Intermediate 9 (34 mg, 99% purity, quant.). LC-MS: Rₜ=1.75 min; MS (ESIpos): m/z=976 [M+H]⁺.

### Example S21: Preparation of 4-aminobutyl (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-12-oxo-3,6,9-trioxa-13-azahexadecan-16-oate·trifluoroacetic acid (2/1) (Intermediate 10)

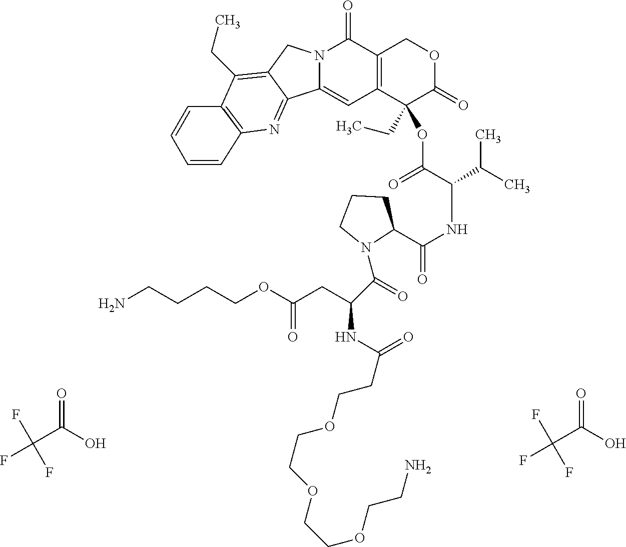

Step 1: (4S)-4,11-Diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Boc-Intermediate 6) (200 mg, 202 μmol) were dissolved in 40 mL DCM and 1.2 eq tert-butyl (4-hydroxybutyl)carbamate (46 mg, 242 μmol) as well as 1.3 eq 1-(3-dimethylaminopropyl)-3-ethylcarbodiimide hydrochloride (50.3 mg, 262 μmol) and 20 mg (164 μmol) 4-(dimethylamino)pyridine were added. After stirring for 20 h at rt the mixture was concentrated in vacuo and the residual was purified by prep. HPLC. Relevant fractions were collected and concentrated in vacuo to yield Boc-Intermediate 10 as a yellow foam (165 mg, 100% purity, 70% yield). LC-MS: Rₜ=5.08 min; MS (ESIpos): m/z=1162 [M+H]⁺.
Step 2: 4-[(tert-butoxycarbonyl)amino]butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2-dimethyl-4,17-dioxo-3,8,11,14-tetraoxa-5,18-diazahenicosan-21-oate (Boc-Intermediate 10) (165 mg, 142 μmol) were dissolved in 30 ml DCM and 6 ml TFA was added and the reaction mixture was stirred for 30 min. It was concentrated in vacuo, the residue was dissolved in ACN/H₂O and lyophilized to give Intermediate 10 (190 mg, 100% purity, quant. yield). LC-MS: Rₜ=2.47 min; MS (ESIpos): m/z=962 [M+H]⁺.

### Example S22: Preparation of (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-valinate·trifluoroacetic acid (1:1) (Intermediate 11)

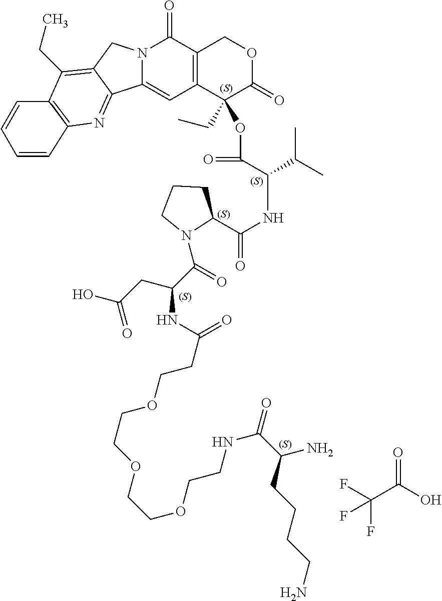

Step 1: (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Boc-Intermediate 6) (500 mg, 498 μmol) was dissolved in 50 mL DMF and 1.2 eq 2,5-dioxopyrrolidin-1-yl N²,N⁶-bis(tert-butoxycarbonyl)-L-lysinate (265 mg, 597 μmol) as well as 260 μL N,N-diisopropylethylamine were added. After stirring for 2 h at rt the mixture was concentrated in vacuo and the residual was purified by prep. HPLC. Relevant fractions were collected and concentrated in vacuo to yield Boc-Intermediate 11 as a light yellow foam (506 mg, 98% purity, 81% yield). LC-MS: Rₜ=1.93 min; MS (ESIpos): m/z=1218 [M+H]⁺.
Step 2: 506 mg (415 μmol) of Boc-Intermediate 11 was dissolved in 10 ml DCM and 3 ml TFA was added and the reaction mixture was stirred for 30 min at rt. It was concentrated in vacuo, the residue was dissolved in ACN/H₂O and lyophilized to give Intermediate 11 as a light yellow foam. (700 mg (100% purity, quant.). LC-MS: Rₜ=1.77 min; MS (ESIpos): m/z=510 [M+2H]++

### Example S23: Preparation of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-valinate·trifluoroacetic acid (½) (Intermediate 12)

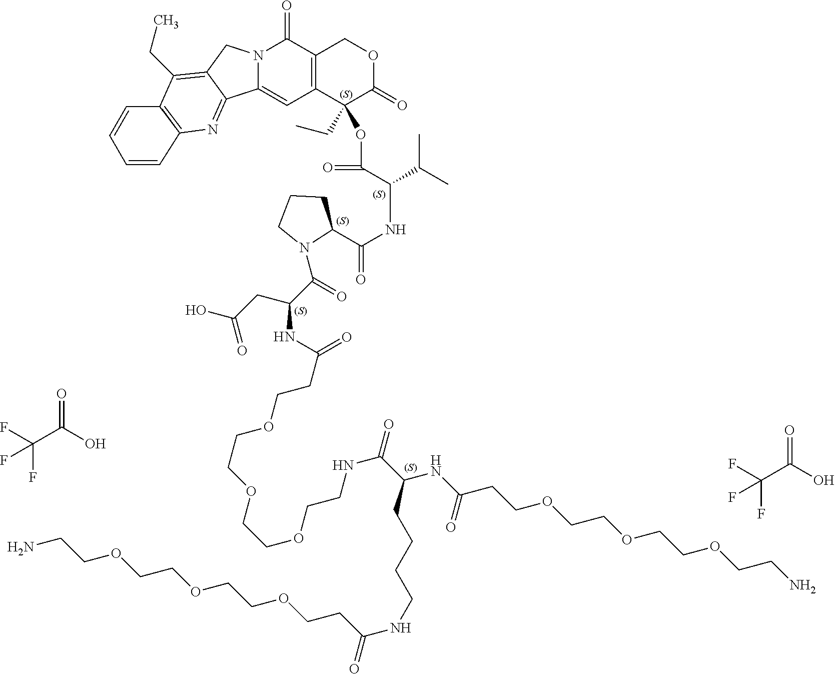

Step 1: (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-valinate (Intermediate 11) (70 mg, 62 μmol) was dissolved in 8 mL DMF and 2.2 eq tert-butyl {2-[2-(2-{3-[(2,5-dioxopyrrolidin-1-yl)oxy]-3-oxopropoxy}ethoxy)ethoxy]ethyl}carbamate (56.9 mg, 136 μmol) (Intermediate 18) as well as 65 μL N,N-diisopropylethylamine were added. After stirring for 4 h at rt the mixture was concentrated in vacuo and the residual was purified by prep. HPLC. Relevant fractions were collected and concentrated in vacuo to yield (Boc-Intermediate 12) as a light yellow foam (60 mg, 87% purity, 52% yield).
Step 2: 60 mg (37 μmol) of Boc-Intermediate 12 were dissolved in 10 ml DCM and 2 ml TFA was added and the reaction mixture was stirred for 30 min at rt. It was concentrated in vacuo, the residue was dissolved in ACN/H₂O and lyophilized to give Intermediate 12 as a colorless foam. (60 mg, 100% purity, 98% yield). LC-MS: Rₜ=1.86 min; MS (ESIpos): m/z=1425 [M+H]⁺.

### Example S24: Preparation of (4S)-4,11-Diethyl-3,14-dioxo-3,4,12,14-tetrahydro-1H-pyrano[3′,4′:6,7]indolizino[1,2-b]quinolin-4-yl N-{3-[2-(2-{2-[(N²,N⁶-di-beta-alanyl-L-lysyl)amino]ethoxy} ethoxy)ethoxy] propanoyl}-L-alpha-aspartyl-L-prolyl-L-valinate (Intermediate 13)

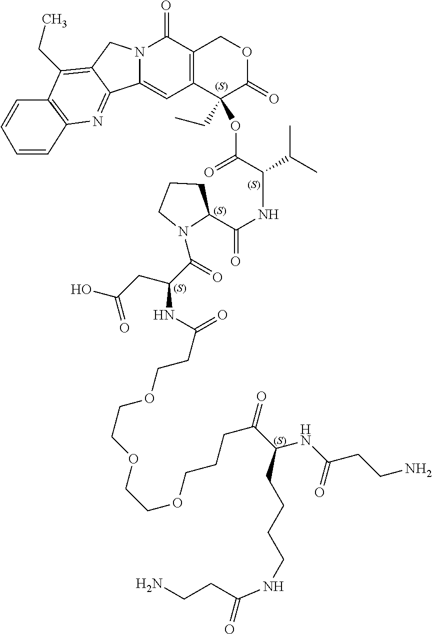

Step 1: (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-valinate·trifluoroacetic acid (1:1) (Intermediate 11) (250 mg, 221 μmol) was dissolved in 20 mL DMF and 2.2 eq 2,5-dioxopyrrolidin-1-yl N-(tert-butoxycarbonyl)-beta-alaninate (139 mg, 485 μmol) as well as 230 μL N,N-diisopropylethylamine were added. After stirring for 1 h at rt the mixture was concentrated in vacuo and the residual was purified by prep. HPLC. Relevant fractions were collected and concentrated in vacuo to yield Boc-Intermediate 13 as a light yellow foam (204 mg, 100% purity, 68% yield). LC-MS: Rₜ=3.16 min; MS (ESIpos): m/z=1361 [M+H]⁺.
Step 2: 204 mg (150 μmol) of Boc-Intermediate 13 were dissolved in 20 ml DCM and 2 ml TFA was added and the reaction mixture was stirred for 30 min at rt. It was concentrated in vacuo, the residue was dissolved in ACN/H₂O and lyophilized to give Intermediate 13 as a light yellow foam. (174 mg; 100% purity, quant.). LC-MS: Rₜ=1.78 min; MS (ESIpos): m/z=1161 [M+H]⁺.

### Example S25: Preparation of (4S)-4,11-diethyl-3,14-dioxo-3,4,12,14-tetrahydro-1H-pyrano[3′,4′:6,7]indolizino[1,2-b]quinolin-4-yl N-methyl-N-[2-(methyl{2-[methyl(2-{[N-methyl-N-(2,2,8,11-tetramethyl-4-oxo-3-oxa-5,8,11-triazatridecan-13-yl)glycyl]amino}ethyl)amino] ethyl}amino)ethyl]glycyl-L-asparaginyl-L-prolyl-L-valinate (Intermediate 14)

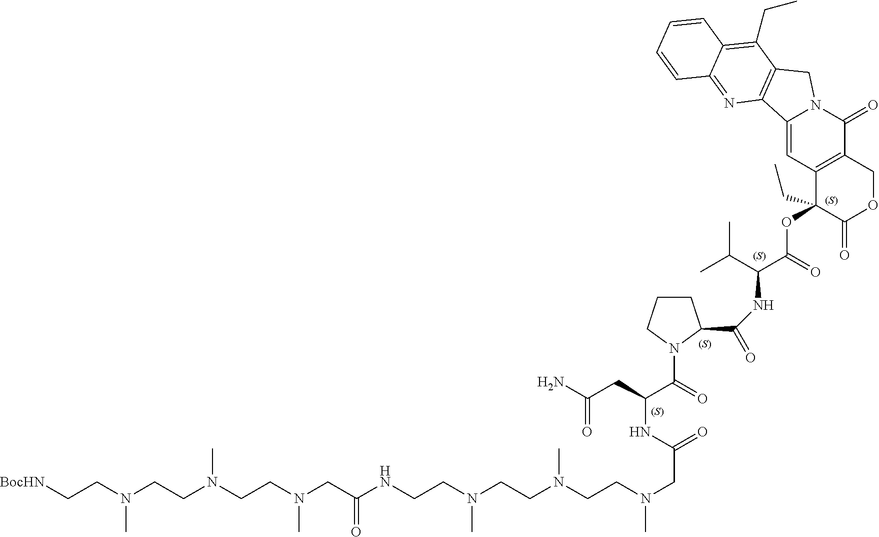

Trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[(2-aminoethyl)(methyl) amino]ethyl}(methyl)amino]ethyl}-N-methylglycyl-L-asparaginyl-L-prolyl-L-valinate (1/1) (Intermediate 4) (65.0 mg, 63.2 μmol) was dissolved in DMF (4.7 mL). N-Methyl-N-(2,2,8,11-tetramethyl-4-oxo-3-oxa-5,8,11-triazatridecan-13-yl)glycine (Building Block 7) (26.3 mg, 75.8 μmol), HATU (36.0 mg, 94.7 μmol; CAS-RN:[148893-10-1]) and DIEA (33 μl, 190 μmol; CAS-RN:[7087-68-5]) were added. The reaction was stirred at RT for 22 h and then concentrated in vacuo. (HPLC) The residue was separated by prep. HPLC to give Intermediate 14 (24.5 mg, 95% purity, 30% yield) as a light, yellow foam. LC-MS: Rₜ=1.63 min; MS (ESIpos): m/z=1243 [M+H]⁺.

### Example S26: Preparation of trifluoroacetic acid-(4S)-4,11-diethyl-3,14-dioxo-3,4,12,14-tetrahydro-1H-pyrano[3′,4′:6,7] indolizino [1,2-b]quinolin-4-yl N-{2-[{2-[{2-[(N-{2-[{2-[(2-aminoethyl)(methyl) amino]ethyl} (methyl) amino]ethyl}-N-methylglycyl)amino]ethyl}(methyl)amino] ethyl}(methyl) amino]ethyl}-N-methylglycyl-L-asparaginyl-L-prolyl-L-valinate (1/1) (Intermediate 15)

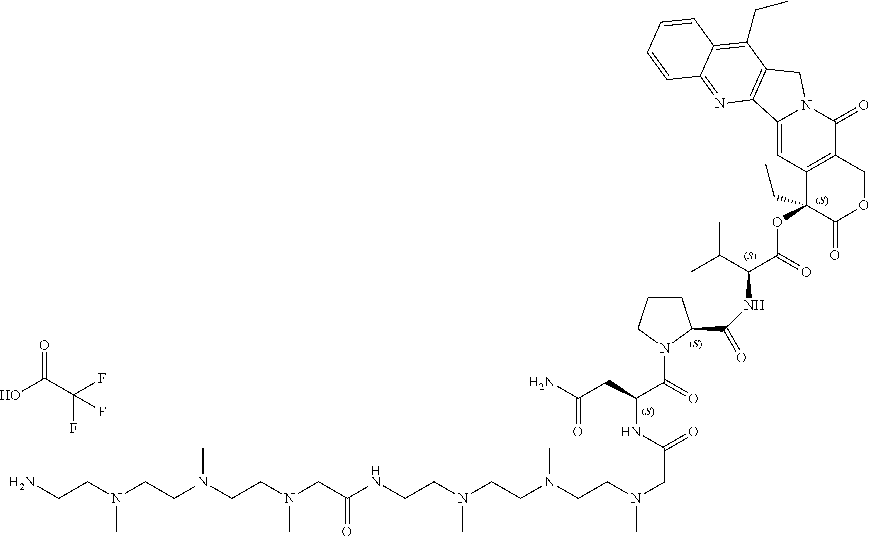

(4S)-4,11-Diethyl-3,14-dioxo-3,4,12,14-tetrahydro-1H-pyrano[3′,4′:6,7]indolizino [1,2-b]quinolin-4-yl N-methyl-N-[2-(methyl{2-[methyl(2-{[N-methyl-N-(2,2,8,11-tetramethyl-4-oxo-3-oxa-5,8,11-triazatridecan-13-yl)glycyl]amino}ethyl)amino]ethyl}amino)ethyl]glycyl-L-asparaginyl-L-prolyl-L-valinate (Intermediate 14) (24.5 mg, 19.7 μmol) was dissolved in DCM (10 mL), then TFA (2.0 mL) was added. The reaction was stirred for 2 h at RT and the concentrated in vacuo. The residue was dissolved in ACN/water and freeze-dried to give Intermediate 15 (26.0 mg, 88% purity, 93% yield) as a yellow foam. LC-MS: Rₜ=1.88 min; MS (ESIpos): m/z=1143 [M+H]⁺.

### Example S27: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(tert-butoxycarbonyl)-L-lysyl]amino}ethyl)(methyl) amino] ethyl}(methyl)amino]ethyl}-N-methylglycyl-L-asparaginyl-L-prolyl-L-valinate (Intermediate 16)

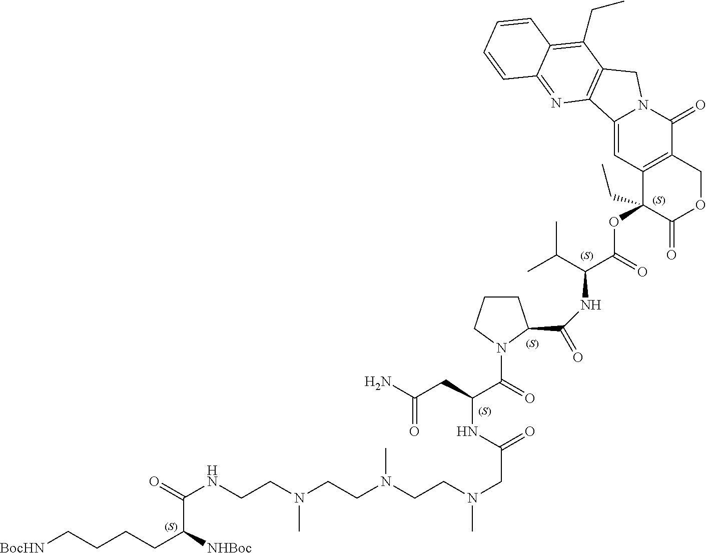

Trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[(2-aminoethyl)(methyl) amino]ethyl}(methyl)amino]ethyl}-N-methylglycyl-L-asparaginyl-L-prolyl-L-valinate (1/1) (Intermediate 4) (60.0 mg, 58.3 μmol) was dissolved in DMF (12 mL). 2,5-Dioxopyrrolidin-1-yl N²,N⁶-bis(tert-butoxycarbonyl)-L-lysinate (33.6 mg, 75.8 μmol) and DIEA (30 μl, 170 μmol) were added. The reaction was stirred at RT for 4 h and then concentrated in vacuo. The residue was separated by prep. HPLC to give Intermediate 16 (72.8 mg, 99% purity, 99% yield) as a light, yellow foam. LC-MS: Rₜ=2.42 min; MS (ESIpos): m/z=1243 [M+H]⁺.

### Example S28: Preparation of trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[(2-{[2-(L-lysylamino)ethyl](methyl)amino} ethyl)(methyl) amino] ethyl}-N-methylglycyl-L-asparaginyl-L-prolyl-L-valinate (2/1) (Intermediate 17)

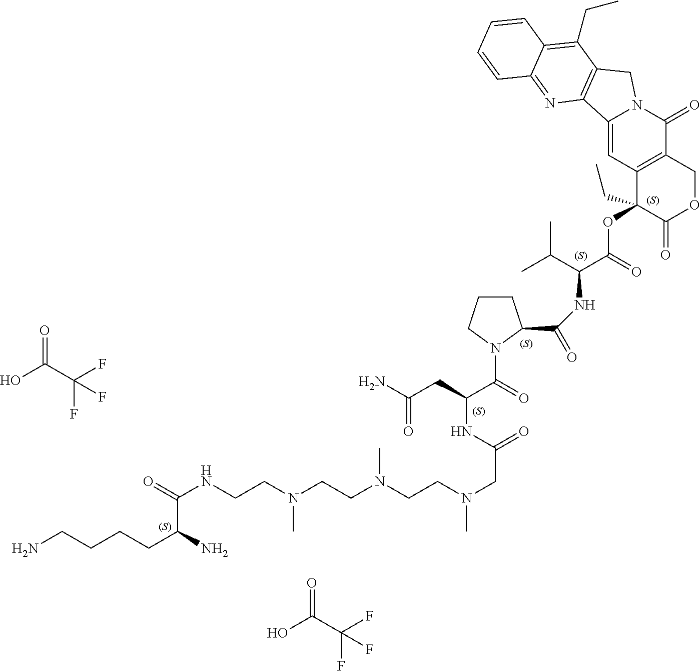

(4S)-4,11-Diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(tert-butoxycarbonyl)-L-lysyl]amino}ethyl)(methyl) amino]ethyl} (methyl) amino]ethyl}-N-methylglycyl-L-asparaginyl-L-prolyl-L-valinate (72.8 mg, 58.5 μmol) was dissolved in DCM (15 mL), then TFA (3.0 mL) was added. The reaction was stirred for 2 h at RT and then concentrated in vacuo. (HPLC). The residue was dissolved in ACN/water and freeze-dried to give Intermediate 17 (80.0 mg, 100% purity, 107% yield) as a yellow foam. LC-MS: Rₜ=1.87 min; MS (ESIpos): m/z=1043 [M+H]⁺.

### Example S29: Preparation of tert-butyl {2-[2-(2-{3-[(2,5-dioxopyrrolidin-1-yl)oxy]-3-oxopropoxy}ethoxy)ethoxy]ethyl}carbamate (Intermediate 18)

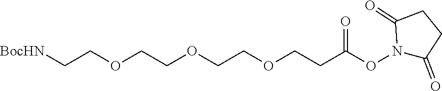 [SMILES: CC(C)(C)OC(=O)NCCOCCOCCOCCC(=O)ON1C(=O)CCC1=O]

To a suspension of 2,2-dimethyl-4-oxo-3,8,11,14-tetraoxa-5-azaheptadecan-17-oic acid (3.00 g, 9.33 mmol) in DCM (50 ml), were added 1-hydroxypyrrolidine-2,5-dione (1.61 g, 14.0 mmol), EDCI (2.15 g, 11.2 mmol) and DMAP (5.00 mg, 40.9 μmol). The reaction was stirred for 1 hour at RT and concentrated in vacuo. The residue was purified by preparative HPLC to give Intermediate 40 (3.15 g, 90% purity, 72% yield) as a colorless oil. LC-MS: Rt=1.38 min; MS (ESIpos): m/z=419 [M+H]⁺.

### Example S30: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(2,2-dimethyl-4,17-dioxo-3,8,11,14-tetraoxa-5-azaheptadecan-17-yl)-L-lysyl]amino}ethyl)(methyl)amino]ethyl}(methyl)amino]ethyl}-N-methylglycyl-L-asparaginyl-L-prolyl-L-valinate (Intermediate 19)

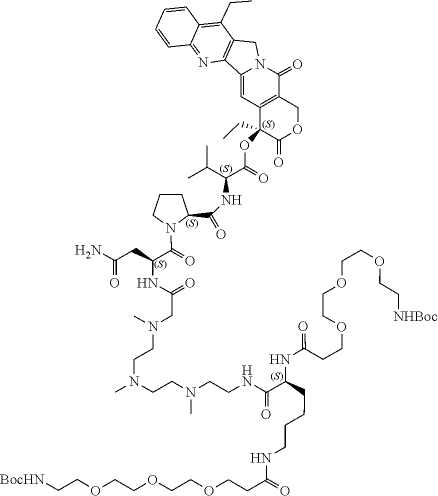

Trifluoroacetic acid-(4S)-4,11-diethyl-3,14-dioxo-3,4,12,14-tetrahydro-1H-pyrano[3′,4′:6,7]indolizino[1,2-b]quinolin-4-yl N-{2-[(2-{[2-(L-lysylamino)ethyl](methyl) amino}ethyl)(methyl)amino]ethyl}-N-methylglycyl-L-asparaginyl-L-prolyl-L-valinate (2/1) (Intermediate 17) (40.0 mg, 31.5 μmol) was dissolved in DMF (10 mL). Tert-butyl {2-[2-(2-{3-[(2,5-dioxopyrrolidin-1-yl)oxy]-3-oxopropoxy}ethoxy)ethoxy]ethyl}carbamate (Intermediate 18) (31.6 mg, 75.5 μmol) and DIEA (44 μl, 250 μmol) were added. The reaction was stirred at RT for 3 h and then concentrated in vacuo. The residue was purified by prep. HPLC to give Intermediate 19 (57.0 mg, 92% purity, 100% yield) as a colorless foam. LC-MS: Rₜ=2.48 min; MS (ESIpos): m/z=1649 [M+H]⁺.

### Example S31: Preparation of trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[(2-{[N²,N⁶-bis(3-{2-[2-(2-aminoethoxy)ethoxy] ethoxy} propanoyl)-L-lysyl]amino}ethyl)(methyl)amino]ethyl}(methyl)amino]ethyl}-N-methylglycyl-L-asparaginyl-L-prolyl-L-valinate (2/1) (Intermediate 20)

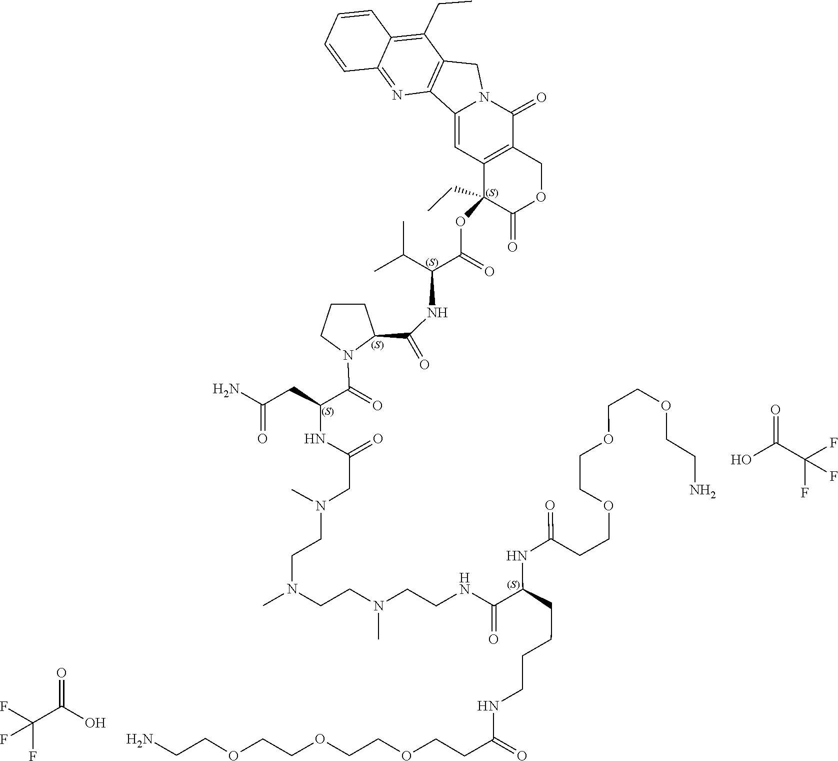

(4S)-4,11-Diethyl-3,14-dioxo-3,4,12,14-tetrahydro-1H-pyrano[3′,4′:6,7]indolizino [1,2-b]quinolin-4-yl N-{2-[{2-[(2-{[N²,N⁶-bis(2,2-dimethyl-4,17-dioxo-3,8,11,14-tetraoxa-5-azaheptadecan-17-yl)-L-lysyl]amino}ethyl)(methyl)amino]ethyl}(methyl)amino]ethyl}-N-methylglycyl-L-asparaginyl-L-prolyl-L-valinate (Intermediate 19) (57.0 mg, 34.5 μmol) was dissolved in DCM (8.0 mL), then TFA (2.0 mL) was added. The reaction was stirred for 30 min at RT and then concentrated in vacuo. (HPLC). The residue was dissolved in ACN/water and freeze-dried to give Intermediate 20 (56.0 mg, 92% purity, 89% yield) as a yellow foam. LC-MS: Rₜ=1.33 min; MS (ESIpos): m/z=1449 [M+H]⁺.

### Example S32: Preparation of (4S)-4,11-diethyl-3,14-dioxo-3,4,12,14-tetrahydro-1H-pyrano[3′,4′:6,7]indolizino[1,2-b]quinolin-4-yl N-{2-[(2-{[2-({N²,N⁶-bis[N-methyl-N-(2,2,8,11-tetramethyl-4-oxo-3-oxa-5,8,11-triazatridecan-13-yl)glycyl]-L-lysyl}amino)ethyl](methyl) amino}ethyl)(methyl)amino]ethyl}-N-methylglycyl-L-asparaginyl-L-prolyl-L-valinate (Intermediate 21)

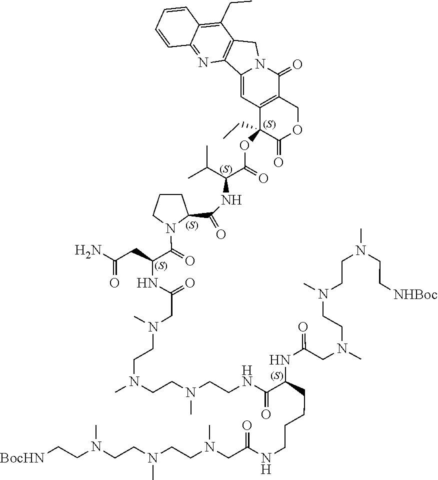

Trifluoroacetic acid-(4S)-4,11-diethyl-3,14-dioxo-3,4,12,14-tetrahydro-1H-pyrano[3′,4′:6,7]indolizino[1,2-b]quinolin-4-yl N-{2-[(2-{[2-(L-lysylamino)ethyl](methyl) amino}ethyl)(methyl)amino]ethyl}-N-methylglycyl-L-asparaginyl-L-prolyl-L-valinate (2/1) (Intermediate 17) (40.0 mg, 31.5 μmol) was dissolved in DMF (5.0 mL). N-Methyl-N-(2,2,8,11-tetramethyl-4-oxo-3-oxa-5,8,11-triazatridecan-13-yl)glycine (Building Block 7) (26.2 mg, 75.5 μmol), HATU (35.9 mg, 94.4 μmol) and DIEA (44 μl, 250 μmol) were added. The reaction was stirred at RT for 1 h and then concentrated in vacuo. The residue was separated by prep. HPLC to give the Intermediate 21 (33.0 mg, 83% purity, 51% yield) as a light, yellow foam. LC-MS: Rₜ=1.44 min; MS (ESIpos): m/z=1701 [M+H]⁺.

### Example S33: Preparation of trifluoroacetic acid-(4S)-4,11-diethyl-3,14-dioxo-3,4,12,14-tetrahydro-1H-pyrano[3′,4′:6,7] indolizino [1,2-b]quinolin-4-yl N-{2-[{2-[(2-{[N2N⁶-bis(N-{2-[{2-[(2-aminoethyl)(methyl) amino]ethyl} (methyl)amino]ethyl}-N-methylglycyl)-L-lysyl]amino}ethyl)(methyl)amino] ethyl} (methyl) amino]ethyl}-N-methylglycyl-L-asparaginyl-L-prolyl-L-valinate (2/1) (Intermediate 22)

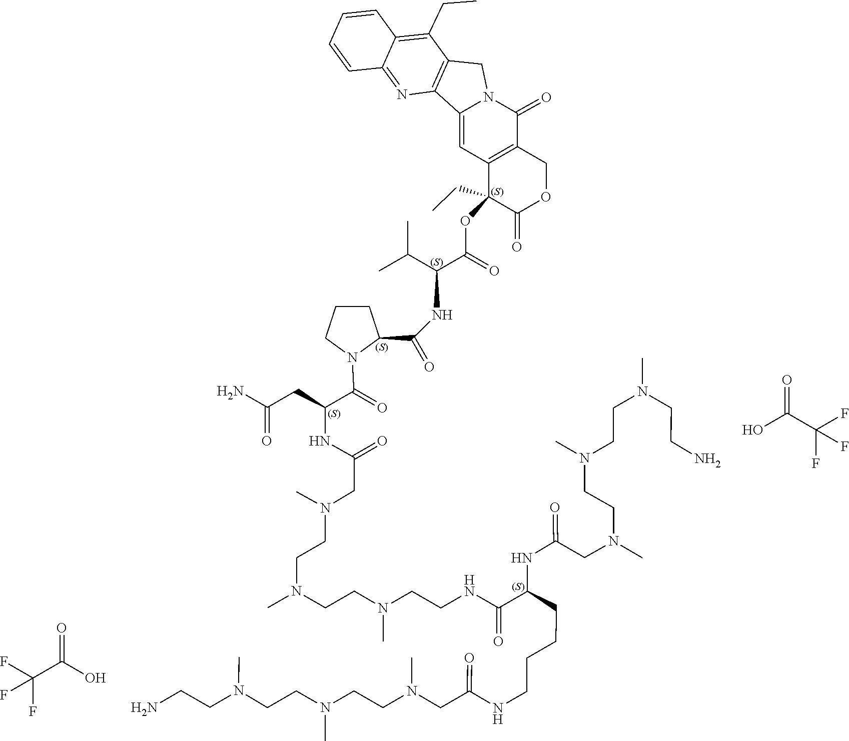

(4S)-4,11-Diethyl-3,14-dioxo-3,4,12,14-tetrahydro-1H-pyrano[3′,4′:6,7]indolizino [1,2-b]quinolin-4-yl N-{2-[(2-{[2-({N²,N⁶-bis [N-methyl-N-(2,2,8,11-tetramethyl-4-oxo-3-oxa-5,8,11-triazatridecan-13-yl) glycyl]-L-lysyl}amino)ethyl](methyl)amino}ethyl)(methyl) amino]ethyl}-N-methylglycyl-L-asparaginyl-L-prolyl-L-valinate (Intermediate 21) (33.0 mg, 19.4 μmol) was dissolved in DCM (5.0 mL), then TFA (1.0 mL) was added. The reaction was stirred for 30 min at RT and then concentrated in vacuo. (HPLC). The residue was dissolved in ACN/water, freeze-dried and then separated by prep. HPLC to give Intermediate 22 (16.5 mg, 100% purity, 49% yield) as a light, yellow foam. LC-MS: Rₜ=1.00 min; MS (ESIpos): m/z=1499 [M+H]r.

### Example S34: Preparation of (4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (Intermediate 23)

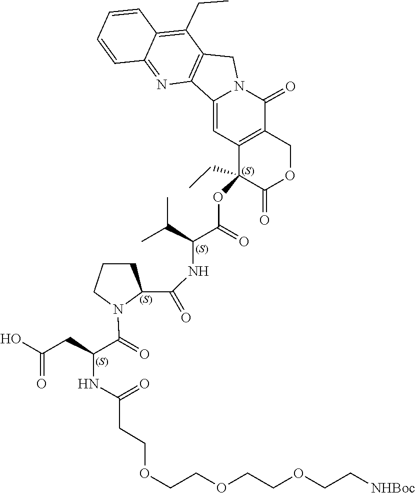

To a solution of (4S)-4,11-diethyl-3,14-dioxo-3,4,12,14-tetrahydro-TH-pyrano[3′,4′:6,7] indolizino[1,2-b]quinolin-4-yl N-(3-{2-[2-(2-aminoethoxy)ethoxy]ethoxy}propanoyl)-L-alpha-aspartyl-L-prolyl-L-valinate·trifluoroacetic acid (1/1) (1.00 g, 995 μmol) (Intermediate 6) in DMF (50 ml) was added Boc-Osu (1-[(tert-butoxycarbonyl)oxy]pyrrolidine-2,5-dione) (428 mg, 1.99 mmol) and N,N-diisopropylethylamine (350 μl, 2.0 mmol). The reaction was stirred for 2 hours at RT, concentrated in vacuo. The residue was purified by prep. HPLC and lyophilized to give Intermediate 23 (715 mg, 100% purity, 73% yield) as a yellow amorphous residue. LC-MS: Rₜ=1.78 min; MS (ESIpos): m/z=991 [M+H]f.

### Example S35: Preparation of (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N,2,2-pentamethyl-4,17,21-trioxo-3,8,11,14,22-pentaoxa-5,18-diazapentacosan-25-aminium trifluoroacetate (Intermediate 24)

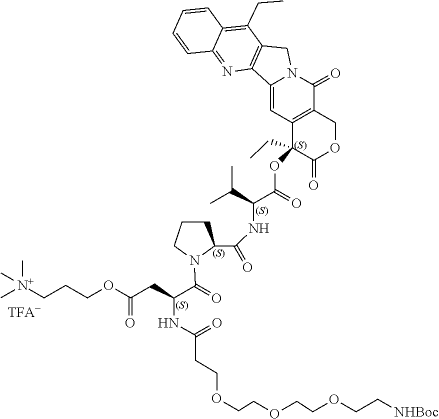

(4S)-4,11-diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-valinate (40.0 mg, 40.4 μmol) (Boc-Intermediate 6) was dissolved in DCM (10 mL), then choline chloride (3-hydroxy-N,N,N-trimethylpropan-1-aminium chloride) (14.9 mg, 96.9 μmol), sodium hydrogen carbonate (8.14 mg, 96.9 μmol), EDCI (20.1 mg, 105 μmol) and DMAP (4.93 mg, 40.4 μmol) were added. The reaction was stirred overnight at RT. Since the reaction had not gone to completion, 1 eq each of choline chloride, NaHCO₃, EDCI and 2 mg DMAP were added. The reaction was stirred over the weekend at RT, concentrated in vacuo and the residue was purified by prep. HPLC and lyophilized to give Intermediate 24 (19.5 mg, 91% purity, 36% yield) as an amorphous residue. LC-MS: Rₜ=3.43 min; MS (ESIpos): m/z=1091 [M+H]⁺.

### Example S36: Preparation of (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N-trimethyl-12,16-dioxo-3,6,9,17-tetraoxa-13-azaicosan-20-aminium trifluoroacetate trifluoroacetic acid (1/1/1) (Intermediate 25)

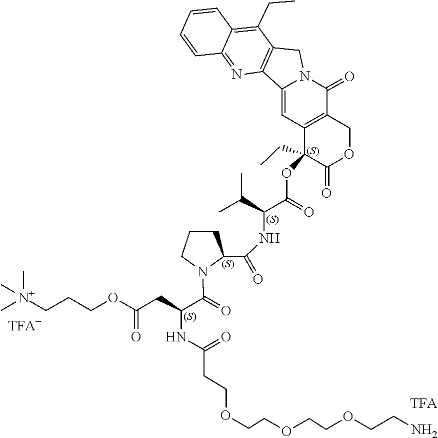

(19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N,2,2-pentamethyl-4,17,21-trioxo-3,8,11,14,22-pentaoxa-5,18-diazapentacosan-25-aminium trifluoroacetate (19.5 mg, 91% purity, 14.7 μmol) (Intermediate 24) was dissolved in DCM (5.0 ml), trifluoroacetic acid (1.0 ml) was added and the reaction was stirred at RT for 30 min. The reaction was then concentrated in vacuo, dissolved in ACN/H₂O, and lyophilized to give Intermediate 25 (20.0 mg, 77% purity, 86% yield) as an amorphous residue. LC-MS: Rₜ=2.40 min; MS (ESIpos): m/z=990 [M+H]⁺.

### Example S37: Preparation of Di-tert-butyl {[(2S)-2-{[(benzyloxy)carbonyl]amino}-1,5-dioxopentane-1,5-diyl]bis (azanediylethane-2,1-diyl)} biscarbamate (Intermediate 26)

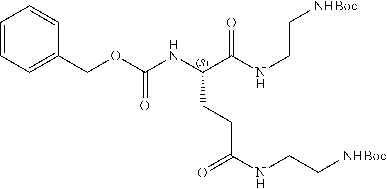

N-[(Benzyloxy)carbonyl]-L-glutamic acid (200 mg, 711 μmol) was dissolved in DMF (20 ml), then N-Boc-ethylenediamine: tert-butyl (2-aminoethyl)carbamate (280 μl, 1.8 mmol), HATU (946 mg, 2.49 mmol) and N,N-diisopropylethylamine (500 μl, 2.8 mmol) were added. The mixture was stirred at RT for 20 min, concentrated in vacuo. The residue was purified by prep. HPLC and lyophilized to give Intermediate 26 (275 mg, 97% purity, 67% yield) as an amorphous residue. LC-MS: (Method 2) Rₜ=1.67 min; MS (ESIpos): m/z=566 [M+H]⁺.

### Example S38: Preparation of trifluoroacetic acid benzyl {(2S)-1,5-bis[(2-aminoethyl)amino]-1,5-dioxopentan-2-yl}carbamate (2/1) (Intermediate 27)

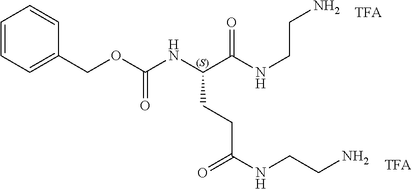

Di-tert-butyl {[(2S)-2-{[(benzyloxy)carbonyl]amino}-1,5-dioxopentane-1,5-diyl]bis(azanediylethane-2,1-diyl)}biscarbamate (275 mg, 97% purity, 474 μmol) (Intermediate 26) was dissolved in DCM (20 ml), TFA (5.0 ml) was added and the reaction was stirred at RT for 30 min. The reaction was concentrated in vacuo, dissolved in ACN/H₂O and lyophilized to give Intermediate 27 (300 mg, 99% purity, quantitative yield) as a resinous compound. LC-MS: Rₜ=0.30 min; MS (ESIpos): m/z=366 [M+H]⁺.

### Example S39: Preparation of benzyl di-tert-butyl [(18S)-12,17,21,26-tetraoxo-3,6,9,29,32,35-hexaoxa-13,16,22,25-tetraazaheptatriacontane-1,18,37-triyl]triscarbamate (Intermediate 28)

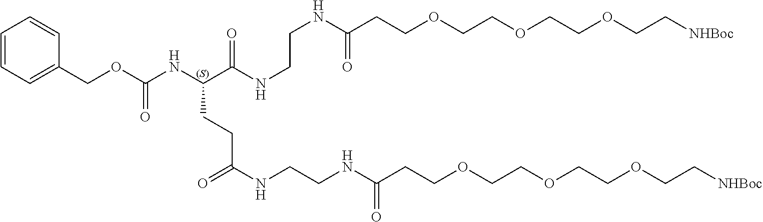

Trifluoroacetic acid benzyl {(2S)-1,5-bis[(2-aminoethyl)amino]-1,5-dioxopentan-2-yl}carbamate (2/1) (120 mg, 99% purity, 201 μmol) (Intermediate 27) was dissolved in DMF (12 ml), then tert-butyl {2-[2-(2-{3-[(2,5-dioxopyrrolidin-1-yl)oxy]-3-oxopropoxy}ethoxy) ethoxy]ethyl}carbamate (336 mg, 804 μmol) (Intermediate 18) and N,N-diisopropylethylamine (180 μl, 1.0 mmol) were added. The mixture was stirred at RT for 2h, concentrated in vacuo, and the residue was purified by prep. HPLC, then lyophilized to give Intermediate 28 (164 mg, 100% purity, 84% yield) as a white amorphous residue. LC-MS: Rₜ=1.58 min; MS (ESIpos): m/z=973 [M+H]⁺

### Example S40: Preparation of tert-butyl (15-{[N-(2,2-dimethyl-4,17-dioxo-3,8,11,14-tetraoxa-5,18-diazaicosan-20-yl)-L-alpha-glutaminyl]amino}-12-oxo-3,6,9-trioxa-13-azapentadecan-1-yl)carbamate (Intermediate 29)

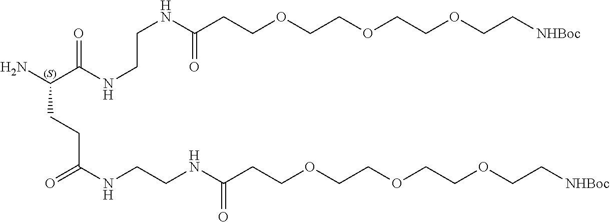

Benzyl di-tert-butyl [(18S)-12,17,21,26-tetraoxo-3,6,9,29,32,35-hexaoxa-13,16,22,25-tetraazaheptatriacontane-1,18,37-triyl]triscarbamate (164 mg, 169 μmol) (Intermediate 28) was dissolved in methanol (20 ml) and in dichloromethane (5.0 ml). Pd/C 10%: (20.0 mg) was added and the reaction was hydrogenated at RT for 1 hour and filtered. The mother liquor was concentrated in vacuo, dissolved in ACN/H₂O and lyophilized to give Intermediate 29 (108 mg, 100% purity, 76% yield) as a resinous compound. LC-MS: Rₜ=2.72 min; MS (ESIpos): m/z=839 [M+H]⁺.

### Example S41: Preparation of (4S)-4,11-diethyl-3,14-dioxo-3,4,12,14-tetrahydro-1H-pyrano[3′,4′:6,7]indolizino[1,2-b]quinolin-4-yl N-{2-[(2-{[2-({5-[(2,5-dioxopyrrolidin-1-yl)oxy]-5-oxopentanoyl}amino)ethyl] (methyl) amino} ethyl)(methyl)amino]ethyl}-N-methylglycylglycyl-L-prolyl-L-valinate (Intermediate 30)

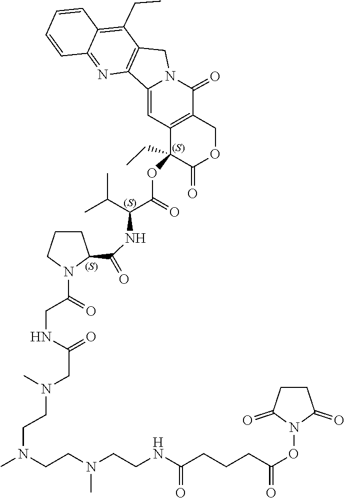

To a solution of trifluoroacetic acid (4S)-4,11-diethyl-3,14-dioxo-3,4,12,14-tetrahydro-1H-pyrano[3′,4′:6,7]indolizino[1,2-b]quinolin-4-yl N-{2-[{2-[(2-aminoethyl)(methyl)amino]ethyl} (methyl)amino]ethyl}-N-methylglycylglycyl-L-prolyl-L-valinate (1/1) (34.0 mg, 96% purity, 33.6 μmol) (Intermediate 5) in DMF (6.0 ml) was added di(N-succinimidyl) glutarate: 1,1′-[(1,5-dioxopentane-1,5-diyl)bis(oxy)]di(pyrrolidine-2,5-dione) (32.9 mg, 101 μmol) and N,N-diisopropylethylamine (23 μl, 130 μmol). The mixture was stirred at RT for 1.5 hours and concentrated in vacuo. The residue was purified by prep. HPLC and lyophilized to give Intermediate 30 (26.5 mg, 79% purity, 58% yield) as an amorphous residue. LC-MS: Rₜ=2.71 min; MS (ESIpos): m/z=1069 [M+H]⁺.

### Example S42: Preparation of (4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(25S)-25-[(2,2-dimethyl-4,17-dioxo-3,8,11,14-tetraoxa-5,18-diazaicosan-20-yl)carbamoyl]-2,2,35,38,41-pentamethyl-4,17,22,27,31,43,46-heptaoxo-3,8,11,14-tetraoxa-5,18,21,26,32,35,38,41,44-nonaazahexatetracontan-46-yl}pyrrolidine-2-carbonyl]amino}-3-methylbutanoate (Intermediate 31)

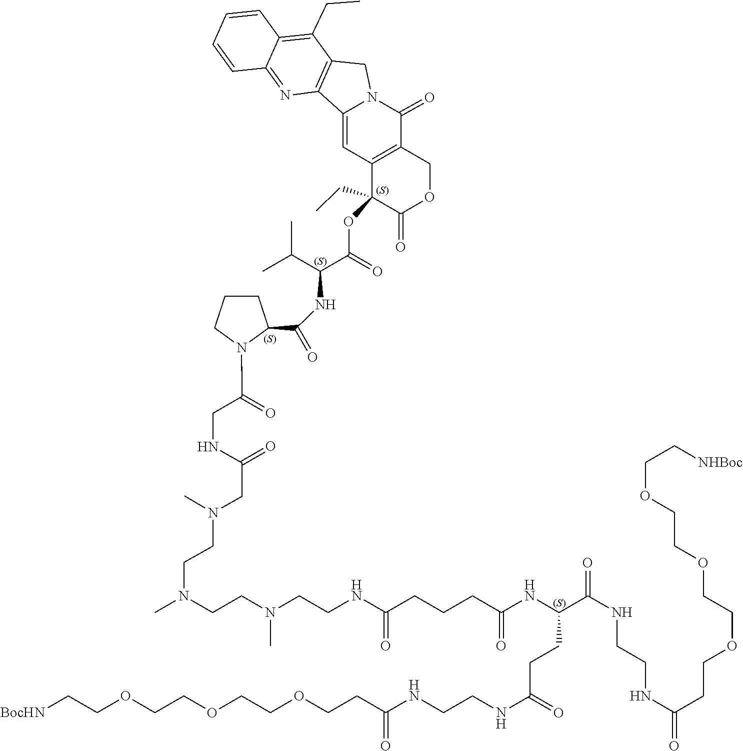

To a solution of (4S)-4,11-diethyl-3,14-dioxo-3,4,12,14-tetrahydro-1H-pyrano[3′,4′:6,7]indolizino[1,2-b]quinolin-4-yl N-{2-[(2-{[2-({5-[(2,5-dioxopyrrolidin-1-yl)oxy]-5-oxopentanoyl}amino)ethyl](methyl)amino}ethyl)(methyl)amino]ethyl}-N-methylglycylglycyl-L-prolyl-L-valinate (26.0 mg, 79% purity, 19.2 μmol) (Intermediate 30) in DMF (5.0 ml) were added tert-butyl (15-{[N-(2,2-dimethyl-4,17-dioxo-3,8,11,14-tetraoxa-5,18-diazaicosan-20-yl)-L-glutaminyl]amino}-12-oxo-3,6,9-trioxa-13-azapentadecan-1-yl)carbamate (16.1 mg, 19.2 μmol) (Intermediate 29) and N,N-diisopropylethylamine (17 μl, 96 μmol; CAS-RN:[7087-68-5]). The reaction was stirred for 24 hours at RT, then concentrated in vacuo. The residue was purified by prep. HPLC and lyophilized to give Intermediate 31 (21.0 mg, 100% purity, 61% yield) as an amorphous residue. LC-MS: Rₜ=3.18 min; MS (ESIpos): m/z=1791 [M+H]⁺.

### Example S43: Preparation of trifluoroacetic acid (4S)-4,11-diethyl-3,14-dioxo-3,4,12,14-tetrahydro-1H-pyrano[3′,4′:6,7] indolizino [1,2-b]quinolin-4-yl (2S)-2-{[(2S)-1-{(22S)-41-amino-22-[(15-amino-4-oxo-7,10,13-trioxa-3-azapentadecan-1-yl)carbamoyl]-6,9,12-trimethyl-4,16,20,25,30-pentaoxo-33,36,39-trioxa-3,6,9,12,15,21,26,29-octaazahentetracontanan-1-oyl}pyrrolidine-2-carbonyl]amino}-3-methylbutanoate (2/1) (Intermediate 32)

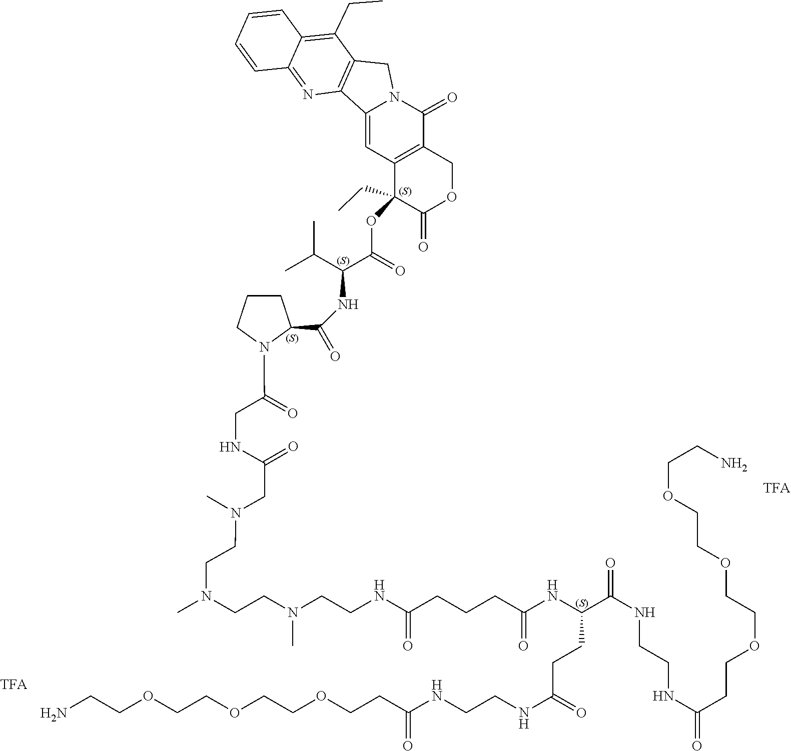

(4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(25S)-25-[(2,2-dimethyl-4,17-dioxo-3,8,11,14-tetraoxa-5,18-diazaicosan-20-yl)carbamoyl]-2,2,35,38,41-pentamethyl-4,17,22,27,31,43,46-heptaoxo-3,8,11,14-tetraoxa-5,18,21,26,32,35,38,41,44-nonaazahexatetracontan-46-yl}pyrrolidine-2-carbonyl]amino}-3-methylbutanoate (21.0 mg, 11.7 μmol) (Intermediate 31) was dissolved in DCM (8.0 ml) and TFA (1.0 ml) was added. The reaction was stirred at RT for 30 min, concentrated in vacuo, dissolved in ACN/H₂O and lyophilized to give Intermediate 32 (20.0 mg, 90% purity, 84% yield) as an amorphous residue. LC-MS: Rₜ=1.89 min; MS (ESIpos): m/z=1590 [M+H]⁺.

### Example S44: Preparation of N1,N5-bis{2-[(tert-butoxycarbonyl)amino]ethyl}-L-glutamamide (Intermediate 33)

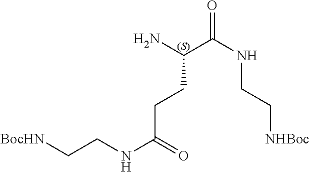

Di-tert-butyl {[(2S)-2-{[(benzyloxy)carbonyl]amino}-1,5-dioxopentane-1,5-diyl]bis (iminoethane-2,1-diyl)}biscarbamate (Intermediate 27) (179.0 mg, 0.316 mmol) was dissolved in methanol (40.0 ml) and dichloromethane (10.0 ml) and Pd/C 10% was added. The mixture was hydrogenated at RT for 1 h. The reaction was filtered, concentrated in vacuo and the residue was dissolved in ACN/H₂O and lyophilized to give Intermediate 33 (129.0 mg, 95% purity, 90% yield) as a white amorphous residue. LC-MS: Rₜ=1.37 min; MS (ESIpos): m/z=431 [M+H]⁺.

### Example S45: Preparation of N1,N5-bis{2-[(tert-butoxycarbonyl)amino]ethyl}-N2-{5-[(2,5-dioxopyrrolidin-1-yl)oxy]-5-oxopentanoyl}-L-glutamamide (Intermediate 34)

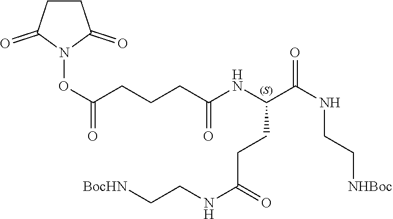

To a solution of N1,N5-bis{2-[(tert-butoxycarbonyl)amino]ethyl}-L-glutamamide (Intermediate 33) (50.0 mg, 95% purity, 110.3 μmol) in DMF (10.0 ml) was added 1,1′-[(1,5-dioxopentane-1,5-diyl)bis(oxy)]dipyrrolidine-2,5-dione (disuccinimidyl glutarate) (72.0 mg, 220.6 umol) and N,N-diisopropylethylamine (76.9 μl, 441.2 μmol). The reaction was stirred at RT for 1 hour, then it was concentrated in vacuo. The residue was purified by prep. HPLC and lyophilized to give Intermediate 34 (36.5 mg, 74% purity, 38% yield) as an amorphous residue. LC-MS: Rₜ=1.35 min; MS (ESIpos): m/z=643 [M+H]⁺.

### Example S46: Preparation of (4S)-4,11-diethyl-3,14-dioxo-3,4,12,14-tetrahydro-1H-pyrano[3′,4′:6,7]indolizino[1,2-b]quinolin-4-yl (2S)-2-[({(2S)-1-[(12S,32S)-32-(2-amino-2-oxoethyl)-12-({2-[(tert-butoxycarbonyl)amino]ethyl}carbamoyl)-2,2,22,25,28-pentamethyl-4,9,14,18,30,33-hexaoxo-3-oxa-5,8,13,19,22,25,28,31-octaazatritriacontan-33-yl]pyrrolidin-2-yl}carbonyl)amino]-3-methylbutanoate (Intermediate 35)

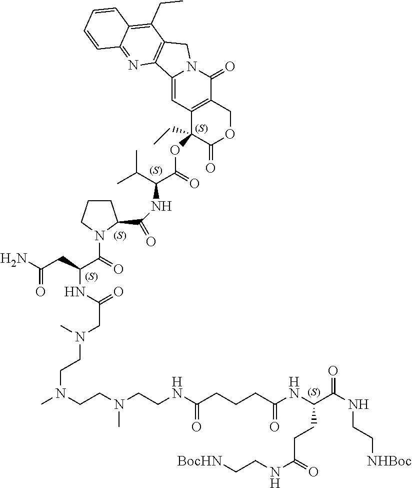

To a solution of Intermediate 4 (15.0 mg. 94% purity, 13.7 μmol) in DMF (6.0 ml) was added Intermediate 34 (15.4 mg, 74% purity, 17.8 μmol) and N,N-Diisopropylethylamine (9.56 μl, 54.9 μmol). The reaction was stirred overnight at RI and concentrated in vacuo. The residue was purified by prep. HPLC, then lyophilized to give Intermediate 35 (15.0 mg, 94% purity, 71% yield) as an amorphous residue. LC-MS: Rₜ=3.01 min; MS (ESIpos): m/z=1441 [M+H]⁺.

### Example S47: Preparation of (4S)-4,11-diethyl-3,14-dioxo-3,4,12,14-tetrahydro-1H-pyrano[3′,4′:6,7]indolizino[1,2-b]quinolin-4-yl (2S)-2-[({(2S)-1-[(12S,32S)-32-(2-amino-2-oxoethyl)-12-({2-[(tert-butoxycarbonyl)amino]ethyl}carbamoyl)-2,2,22,25,28-pentamethyl-4,9,14,18,30,33-hexaoxo-3-oxa-5,8,13,19,22,25,28,31-octaazatritriacontan-33-yl]pyrrolidin-2-yl}carbonyl)amino]-3-methylbutanoate (Intermediate 36)

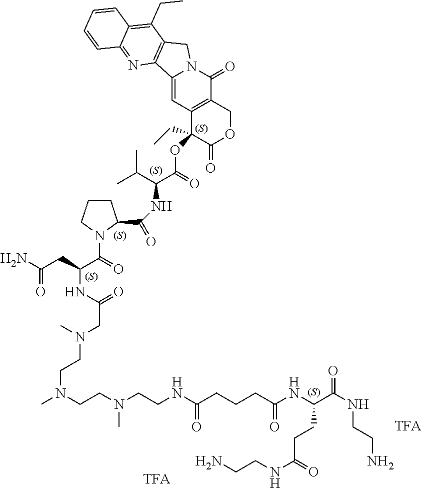

(4S)-4,11-diethyl-3,14-dioxo-3,4,12,14-tetrahydro-1H-pyrano[3′,4′:6,7]indolizino[1,2-b]quinolin-4-yl (2S)-2-[({(2S)-1-[(12S,32S)-32-(2-amino-2-oxoethyl)-12-({2-[(tert-butoxycarbonyl)amino]ethyl}carbamoyl)-2,2,22,25,28-pentamethyl-4,9,14,18,30,33-hexaoxo-3-oxa-5,8,13,19,22,25,28,31-octaazatritriacontan-33-yl]pyrrolidin-2-yl}carbonyl)amino]-3-methylbutanoate (Intermediate 35) (15.0 mg, 94% purity, 9.76 μmol) was dissolved in dichloromethane (5.0 ml), TFA (1.0 ml) was added and the reaction was stirred at RT for 30 min. The reaction was concentrated in vacuo, dissolved in MeCN/H₂O and purified by prep. HPLC to give Intermediate 36 (14.0 mg, 97% purity, 94% yield) as an amorphous residue. LC-MS: Rₜ=1.81 min; MS (ESIpos): m/z=1241 [M+H]⁺.

### Example S48: Preparation of (4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(25S,45S)-45-(2-amino-2-oxoethyl)-25-[(2,2-dimethyl-4,17-dioxo-3,8,11,14-tetraoxa-5,18-diazaicosan-20-yl)carbamoyl]-2,2,35,38,41-pentamethyl-4,17,22,27,31,43,46-heptaoxo-3,8,11,14-tetraoxa-5,18,21,26,32,35,38,41,44-nonaazahexatetracontan-46-yl}pyrrolidine-2-carbonyl]amino}-3-methylbutanoate (Intermediate 37)

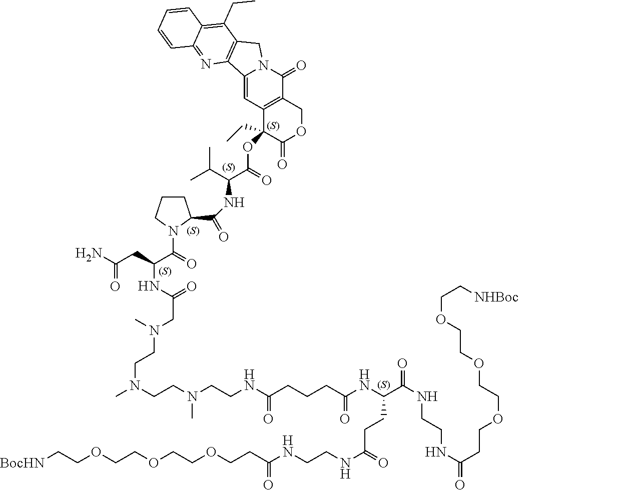

Trifluoroacetic acid (4S)-4,11-diethyl-3,14-dioxo-3,4,12,14-tetrahydro-1H-pyrano[3′,4′:6,7]indolizino[1,2-b]quinolin-4-yl (2S)-2-({(2S)-1-[(2S,22S)-28-amino-22-[(2-aminoethyl)carbamoyl]-2-(2-amino-2-oxoethyl)-6,9,12-trimethyl-4,16,20,25-tetraoxo-3,6,9,12,15,21,26-heptaazaoctacosanan-1-oyl]pyrrolidine-2-carbonyl}amino)-3-methylbutanoate (2/1) (33.0 mg, 8400 purity, 24.7 μmol) (Intermediate 36) was dissolved in DMF (5.0 ml), then tert-butyl {2-[2-(2-{3-[(2,5-dioxopyrrolidin-1-yl)oxy]-3-oxopropoxy}ethoxy)ethoxy]ethyl}carbamate (20.7 mg, 24.7 μmol) (Intermediate 29) and N,N-diisopropylethylamine (17.2 μl, 99 μmol; CAS-RN:[7087-68-5]) were added. The reaction was stirred at RI for 24h, concentrated in vacuo and purified by prep. HPLC to give Intermediate 37 (14.5 mg, 66% purity, 21% yield). LC-MS: Rₜ=3.14 min; MS (ESIpos): m/z=1848 [M+H]⁺.

### Example S49: Preparation of trifluoroacetic acid (4S)-4,11-diethyl-3,14-dioxo-3,4,12,14-tetrahydro-1H-pyrano[3′,4′:6,7] indolizino[1,2-b]quinolin-4-yl (2S)-2-{[(2S)-1-{(2S,22S)-41-amino-2-(2-amino-2-oxoethyl)-22-[(15-amino-4-oxo-7,10,13-trioxa-3-azapentadecan-1-yl)carbamoyl]-6,9,12-trimethyl-4,16,20,25,30-pentaoxo-33,36,39-trioxa-3,6,9,12,15,21,26,29-octaazahentetracontanan-1-oyl}pyrrolidine-2-carbonyl]amino}-3-methylbutanoate (2/1) (Intermediate 38)

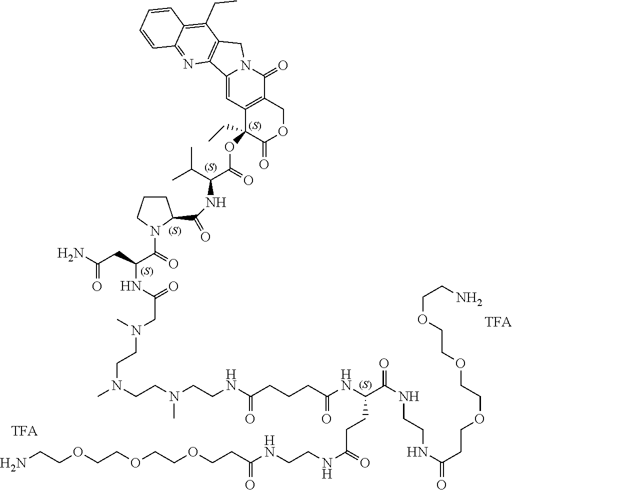

(4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(25S,45S)-45-(2-amino-2-oxoethyl)-25-[(2,2-dimethyl-4,17-dioxo-3,8,11,14-tetraoxa-5,18-diazaicosan-20-yl)carbamoyl]-2,2,35,38,41-pentamethyl-4,17,22,27,31,43,46-heptaoxo-3,8,11,14-tetraoxa-5,18,21,26,32,35,38,41,44-nonaazahexatetracontan-46-yl}pyrrolidine-2-carbonyl]amino}-3-methylbutanoate (14.5 mg, 7.84 μmol) (Intermediate 37) was dissolved in dichloromethane (8.0 ml), trifluoroacetic acid (1.0 ml) was added and the reaction was stirred at RT for 30 min and then concentrated in vacuo. The residue was dissolved in ACN/H₂O and lyophilized to give Intermediate 38 (14.0 mg, 100% purity, 95% yield) as an amorphous residue. LC-MS: Rt=1.90 min; MS (ESIpos): m/z=1647 [M+H]⁺.

### Example S50: (4S)-4,11-diethyl-3,14-dioxo-3,4,12,14-tetrahydro-1H-pyrano[3′,4′:6,7]indolizino[1,2-b]quinolin-4-yl N-{18-[(2,5-dioxopyrrolidin-1-yl)oxy]-14,18-dioxo-4,7,10-trioxa-13-azaoctadecanan-1-oyl}-L-alpha-aspartyl-L-prolyl-L-valinate (Intermediate 39)

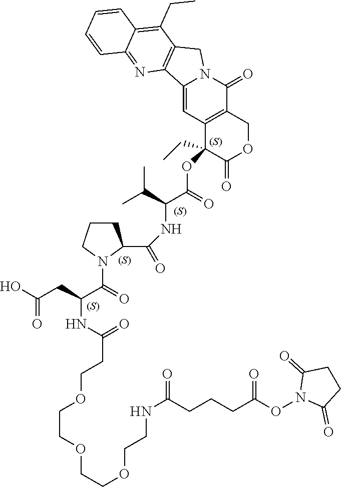

To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate·trifluoroacetic acid (1/1) (123 mg, 123 μmol) (Intermediate 6) in DMF (10 ml) were added 1,1′-[(1,5-dioxopentane-1,5-diyl)bis(oxy)]di(pyrrolidine-2,5-dione) (120 mg, 368 μmol) and DIEA (64 μl, 370 μmol). The mixture was stirred at rt for 30 min and then concentrated under reduced pressure. The residue was purified by preparative HPLC to afford Intermediate 39 (82.0 mg, 98% purity, 59% yield) as a colorless foam. LC-MS (Method 3): Rₜ=3.65 min; MS (ESIpos): m/z=1102 [M+H]⁺.

### Example S51: Preparation of 4S)-4,11-diethyl-3,14-dioxo-3,4,12,14-tetrahydro-1H-pyrano[3′,4′:6,7]indolizino[1,2-b]quinolin-4-yl N-[(5S)-5-amino-1-carboxy-4,11,15,28-tetraoxo-19,22,25-trioxa-3,10,16-triazaoctacosan-28-yl]-L-alpha-aspartyl-L-prolyl-L-valinate·trifluoroacetic acid (1/1) (Intermediate 40)

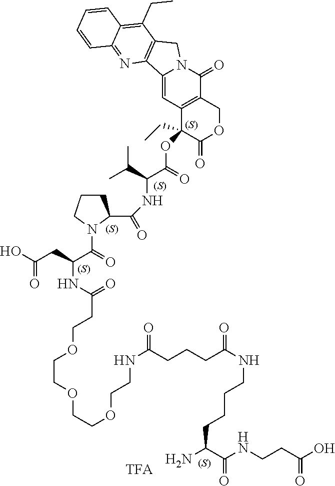

Step 1. To a solution of (4S)-4,11-diethyl-3,14-dioxo-3,4,12,14-tetrahydro-1H-pyrano[3′,4′:6,7]indolizino[1,2-b]quinolin-4-yl N-{18-[(2,5-dioxopyrrolidin-1-yl)oxy]-14,18-dioxo-4,7,10-trioxa-13-azaoctadecanan-1-oyl}-L-alpha-aspartyl-L-prolyl-L-valinate (41.0 mg, 37.2 μmol) (Intermediate 39) in DMF (5 ml) was added tert-butyl N²-(tert-butoxycarbonyl)-L-lysyl-beta-alaninate (20.8 mg, 55.8 gmol) and DIEA (26 μl, 150 μmol). The mixture was stirred at rt for 2 h and then concentrated under reduced pressure. The residue was purified by preparative HPLC to afford ((4S)-4,11-diethyl-3,14-dioxo-3,4,12,14-tetrahydro-1H-pyrano[3′,4′:6,7]indolizino[1,2-b]quinolin-4-yl N-{(9S)-9-[(tert-butoxycarbonyl)amino]-2,2-dimethyl-4,8,15,19,32-pentaoxo-3,23,26,29-tetraoxa-7,14,20-triazadotriacontan-32-yl}-L-alpha-aspartyl-L-prolyl-L-valinate (50 mg, 100% purity, 99% yield) as a yellow foam. LC-MS (Method 3): Rₜ=4.31 min; MS (ESIpos): m/z=1360 [M+H]⁺.
Step 2: To a solution of (4S)-4,11-diethyl-3,14-dioxo-3,4,12,14-tetrahydro-1H-pyrano[3′,4′:6,7]indolizino[1,2-b]quinolin-4-yl N-{(9S)-9-[(tert-butoxycarbonyl)amino]-2,2-dimethyl-4,8,15,19,32-pentaoxo-3,23,26,29-tetraoxa-7,14,20-triazadotriacontan-32-yl}-L-alpha-aspartyl-L-prolyl-L-valinate (50.0 mg, 36.7 μmol) in DCM (5.0 ml), was added TFA (2.5 ml). The mixture was stirred at rt for 1 h and then concentrated under reduced pressure. The residue was dissolved in ACN/H₂O and lyophilized to afford Intermediate 40 (54 mg, 100% purity, 111%) as a yellow foam. LC-MS (Method 3): Rₜ=2.87 min; MS (ESIpos): m/z=1204 [M+H]⁺.

### Example S52: Preparation of (4S)-4,11-diethyl-3,14-dioxo-3,4,12,14-tetrahydro-1H-pyrano[3′,4′:6,7]indolizino[1,2-b]quinolin-4-yl N-{(24S)-37-amino-24-[(2-carboxyethyl) carbamoyl]-14,18,26-trioxo-4,7,10,29,32,35-hexaoxa-13,19,25-triazaheptatriacontanan-1-oyl}-L-alpha-aspartyl-L-prolyl-L-valinate·trifluoroacetic acid (1/1) (Intermediate 41)

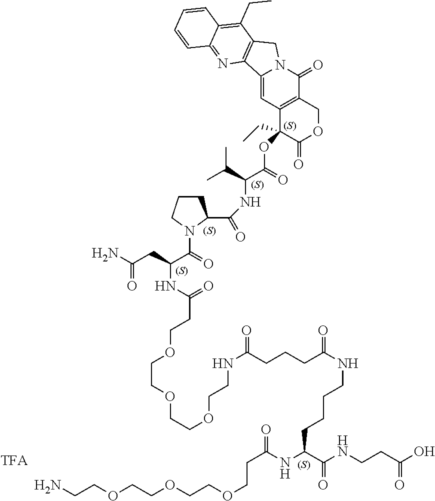

Step 1: To a solution of ((4S)-4,11-diethyl-3,14-dioxo-3,4,12,14-tetrahydro-1H-pyrano[3′,4′:6,7]indolizino[1,2-b]quinolin-4-yl N-[(5S)-5-amino-1-carboxy-4,11,15,28-tetraoxo-19,22,25-trioxa-3,10,16-triazaoctacosan-28-yl]-L-alpha-aspartyl-L-prolyl-L-valinate·trifluoroacetic acid (1/1) (Intermediate 40) (54.0 mg, 41.0 μmol) in DMF (8.6 ml) were added (Intermediate 18) (41.1 mg, 98.3 μmol) and DIEA (57 μl, 330 μmol). The mixture was stirred at rt for 2 h and then concentrated under reduced pressure. The residue was purified by preparative HPLC to afford (4S)-4,11-diethyl-3,14-dioxo-3,4,12,14-tetrahydro-1H-pyrano[3′,4′:6,7]indolizino[1,2-b]quinolin-4-yl N-{(19S)-19-[(2-carboxyethyl)carbamoyl]-2,2-dimethyl-4,17,25,29,42-pentaoxo-3,8,11,14,33,36,39-heptaoxa-5,18,24,30-tetraazadotetracontan-42-yl}-L-alpha-aspartyl-L-prolyl-L-valinate (60 mg, 94% purity, 92% yield) as a yellow foam. LC-MS (Method 3): Rₜ=3.80 min; MS (ESIpos): m/z=1507 [M+H]⁺.
Step 2. To a solution of (4S)-4,11-diethyl-3,14-dioxo-3,4,12,14-tetrahydro-1H-pyrano[3′,4′:6,7]indolizino[1,2-b]quinolin-4-yl N-{(19S)-19-[(2-carboxyethyl)carbamoyl]-2,2-dimethyl-4,17,25,29,42-pentaoxo-3,8,11,14,33,36,39-heptaoxa-5,18,24,30-tetraazadotetracontan-42-yl}-L-alpha-aspartyl-L-prolyl-L-valinate (60.0 mg, 39.8 μmol) in DCM (5.4 ml), was added TFA (2.7 ml). The mixture was stirred at rt for 1 h and then concentrated under reduced pressure. The residue was dissolved in ACN/H₂O and lyophilized to afford Intermediate 41 (62 mg, 95% purity, 97%) as a yellow foam. LC-MS (Method 3): Rₜ=2.85 min; MS (ESIpos): m/z=1407 [M+H]⁺.

### Example S53: Preparation of benzyl (2S)-1-[(2S)-4-tert-butoxy-2-(tert-butoxycarbonylamino)-4-oxo-butanoyl]pyrrolidine-2-carboxylate (Intermediate 42)

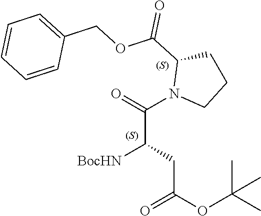

Benzyl (2˜{S})-1-[(2˜{S})-4tert-butoxy-2-(tert-butoxycarbonylamino)-4-oxo-butanoyl]pyrrolidine-2-carboxylate (Intermediate 42) was synthesized using classical methods of peptide synthesis to couple Boc-Asp(OtBu)-OH with benzyl L-prolinate hydrochloride (1:1) in THF in the presence of T3P and DIPEA. LC-MS (Method 2): Rₜ=2.20 min; MS (ESIpos): m/z=477 [M+H]⁺.

### Example S54: Preparation of benzyl (2S)-1-[(2S)-2-amino-4-tert-butoxy-4-oxo-butanoyl]pyrrolidine-2-carboxylate (Intermediate 43)

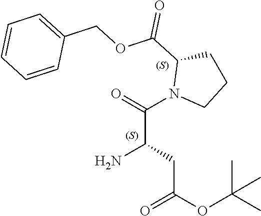

To a solution of benzyl (2S)-1-[(2S)-4-tert-butoxy-2-(tert-butoxycarbonylamino)-4-oxo-butanoyl]pyrrolidine-2-carboxylate (Intermediate 42) (410 mg, 0.86 mmol) in DCM (10 ml), was added TFA (1 ml), The mixture was stirred at rt for 3 h and then concentrated under reduced pressure. The residue was purified over preparative HPLC and freeze dried to afford benzyl (2S)-1-[(2S)-2-amino-4-tert-butoxy-4-oxo-butanoyl]pyrrolidine-2-carboxylate (Intermediate 43) (240 mg, 100% purity, 57% yield) as a colorless foam. LC-MS (Method 5): Rₜ=0.65 min; MS (ESIpos): m/z=377 [M+H]⁺.

### Example S55: Preparation of trifluoroacetic acid-benzyl N-methylglycyl-N-methylglycinate (1/1) (Intermediate 44)

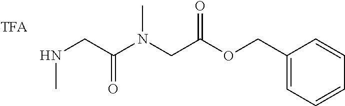 [SMILES: CNCC(=O)N(C)CC(=O)OCc1ccccc1]

Step 1: To a solution of N-(tert-butoxycarbonyl)-N-methylglycyl-N-methylglycine (1.00 g, 3.84 mmol) in DCM (20 ml) were added phenylmethanol (480 μl, 4.6 mmol), EDCI (1.10 g, 5.76 mmol) and DMAP (93.9 mg, 768 μmol). The mixture was then stirred at RT for 1 h and concentrated under reduced pressure. The residue was purified over preparative HPLC to afford benzyl N-(tert-butoxycarbonyl)-N-methylglycyl-N-methylglycinate (1.40 g, 95% purity, 99% yield). LC-MS (Method 2): Rₜ=1.76 min; MS (ESIpos): m/z=351 [M+H]+.
Step 2: To a solution of benzyl N-(tert-butoxycarbonyl)-N-methylglycyl-N-methylglycinate (1.40 g, 95% purity, 3.80 mmol) in DCM (25 ml) was added TFA (5 ml). The mixture was stirred at RT for 30 min and then concentrated under reduced pressure. The residue was dissolved in ACN/H₂O and lyophilized to afford Intermediate 44 (1.40 g, 99% purity, 100% yield). LC-MS (Method 4): Rₜ=0.65 min; MS (ESIpos): m/z=251 [M+H]⁺.

### Example S56: Preparation of benzyl N-(tert-butoxycarbonyl)-N-methylglycyl-N-methylglycyl-N-methylglycyl-N-methylglycinate (Intermediate 45)

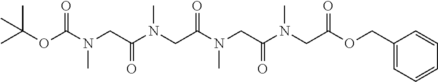 [SMILES: CN(CC(=O)OCc1ccccc1)C(=O)CN(C)C(=O)CN(C)C(=O)CN(C)C(=O)OC(C)(C)C]

To a solution of trifluoroacetic acid-benzyl N-methylglycyl-N-methylglycinate (1/1) (733 mg, 99% purity, 2.00 mmol) in DMF (15 ml) were added N-(tert-butoxycarbonyl)-N-methylglycyl-N-methylglycine (520 mg, 2.00 mmol), HATU (1.14 g, 3.00 mmol) and N,N-diisopropylethylamine (1.0 ml, 6.0 mmol). The mixture was then stirred at RT for 1 h and concentrated under reduced pressure. The residue was purified over preparative HPLC to afford Intermediate 45 (968 mg, 97% purity, 96% yield). LC-MS (Method 2): Rₜ=1.52 min; MS (ESIpos): m/z=493 [M+H]⁺.

### Example S57: Preparation of 2-[[2-[[2-[[2-[tert-butoxycarbonyl(methyl)amino]acetyl]-methyl-amino]acetyl]-methyl-amino]acetyl]-methyl-amino]acetic acid (Intermediate 46)

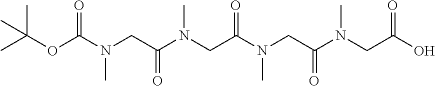 [SMILES: CN(CC(=O)O)C(=O)CN(C)C(=O)CN(C)C(=O)CN(C)C(=O)OC(C)(C)C]

Benzyl N-(tert-butoxycarbonyl)-N-methylglycyl-N-methylglycyl-N-methylglycyl-N-methylglycinate (480 mg, 97% purity, 949 μmol) (Intermediate 45) was dissolved in methanol (40 ml) and dichloromethane (10 ml). Pd/C 10% (40.0 mg) was added and the reaction was hydrogenated at rt for 1 h 30 and filtered. The mother liquor was concentrated in vacuo, dissolved in DCM and evaporated again to afford Intermediate 46 (375 mg. 94% purity, 93% yield) as a white foam. LC-MS (Method 2): Rₜ=0.88 min; MS (ESIpos): m/z=403 [M+H]⁺.

### Example S58: Preparation of (2S)-1-[(18S)-18-(2-tert-butoxy-2-oxoethyl)-2,2,5,8,11,14-hexamethyl-4,7,10,13,16,19-hexaoxo-3-oxa-5,8,11,14,17-pentaazanonadecan-19-yl]pyrrolidine-2-carboxylic acid (Intermediate 47)

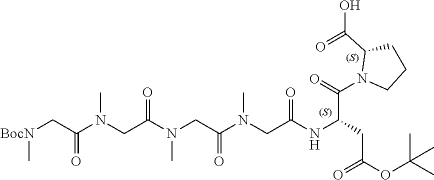

Step 1: To a solution of N-(tert-butoxycarbonyl)-N-methylglycyl-N-methylglycyl-N-methylglycyl-N-methylglycine (Intermediate 46) (50.0 mg, 94% purity, 117 μmol) in DMF (10 ml), were added trifluoroacetic acid-benzyl (2S)-1-[(2S)-2-amino-4-tert-butoxy-4-oxobutanoyl]pyrrolidine-2-carboxylate (1/1) (Intermediate 43) (63.2 mg, 129 μmol), HATU (66.8 mg, 176 μmol) and N,N-diisopropylethylamine (61 μl, 350 μmol). The mixture was stirred at rt for 30 min and then concentrated under reduced pressure. The residue was purified by preparative HPLC and lyophilized to afford benzyl (2S)-1-[(18S)-18-(2-tert-butoxy-2-oxoethyl)-2,2,5,8,11,14-hexamethyl-4,7,10,13,16,19-hexaoxo-3-oxa-5,8,11,14,17-pentaazanonadecan-19-yl]pyrrolidine-2-carboxylate (72.0 mg, 100% purity, 81% yield). LC-MS (Method 5): Rₜ=0.99 min; MS (ESIpos): m/z=761 [M+H]⁺.
Step 2: Benzyl (2S)-1-[(18S)-18-(2-tert-butoxy-2-oxoethyl)-2,2,5,8,11,14-hexamethyl-4,7,10,13,16,19-hexaoxo-3-oxa-5,8,11,14,17-pentaazanonadecan-19-yl]pyrrolidine-2-carboxylate (72.0 mg, 94.6 μmol) was dissolved in methanol (15 ml) and dichloromethane (5 ml). Pd/C 10% (10.0 mg) was added and the reaction was hydrogenated at RT for 1 h and filtered. The mother liquor was concentrated in vacuo, and then lyophilized to afford Intermediate 47 (63 mg, 99% purity, 98% yield) as a white amorphous residue. LC-MS (Method 4): Rₜ=1.39 min; MS (ESIpos): m/z=671 [M+H]⁺.

### Example S59: Preparation of (4S)-4,11-diethyl-3,14-dioxo-3,4,12,14-tetrahydro-1H-pyrano[3′,4′:6,7]indolizino[1,2-b]quinolin-4-yl N-methylglycyl-N-methylglycyl-N-methylglycyl-N-methylglycyl-L-alpha-aspartyl-L-prolyl-L-valinate-trifluoroacetic acid (1/1) (Intermediate 48)

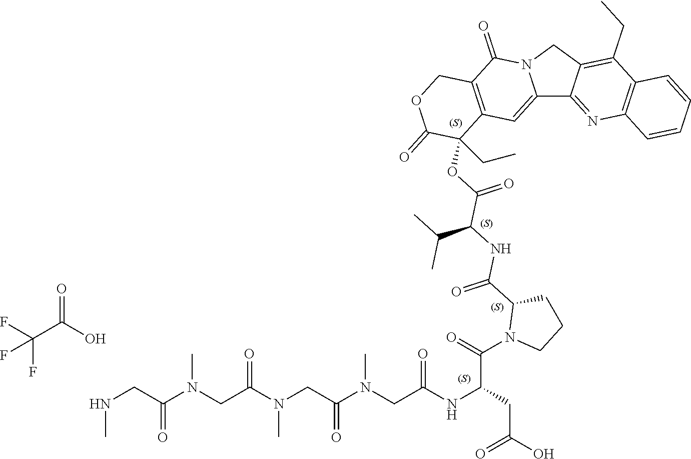

Step 1: To a solution of ((4S)-4,11-diethyl-3,14-dioxo-3,4,12,14-tetrahydro-1H-pyrano[3′,4′:6,7]indolizino[1,2-b]quinolin-4-yl N-[(5S)-5-amino-1-carboxy-4,11,15,28-tetraoxo-19,22,25-trioxa-3,10,16-triazaoctacosan-28-yl]-L-alpha-aspartyl-L-prolyl-L-valinate·trifluoroacetic acid (1/1) (Building Block 9) (30.0 mg, 50.9 μmol) in DMF (8 ml) was added (2S)-1-[(18S)-18-(2-tert-butoxy-2-oxoethyl)-2,2,5,8,11,14-hexamethyl-4,7,10,13,16,19-hexaoxo-3-oxa-5,8,11,14,17-pentaazanonadecan-19-yl]pyrrolidine-2-carboxylic acid (37.5 mg, 56.0 μmol) (Intermediate 47), HATU (29.0 mg, 76.3 μmol) and DIEA (35 μl, 200 μmol). The mixture was stirred at rt for 15 min and then concentrated under reduced pressure. The residue was purified by preparative HPLC to afford tert-butyl (18S)-18-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2,5,8,11,14-hexamethyl-4,7,10,13,16-pentaoxo-3-oxa-5,8,11,14,17-pentaazaicosan-20-oate (55 mg, 100% purity, 96% yield). LC-MS (Method 3): Rₜ=4.61 min; MS (ESIpos): m/z=1129 [M+H]⁺.
Step 2: To a solution of tert-butyl (18S)-18-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2,5,8,11,14-hexamethyl-4,7,10,13,16-pentaoxo-3-oxa-5,8,11,14,17-pentaazaicosan-20-oate (55.0 mg, 48.7 μmol) in DCM (8 ml), was added TFA (2.0 ml). The mixture was stirred at rt for 3 h and then concentrated under reduced pressure. The residue was dissolved in ACN/H₂O and lyophilized to afford Intermediate 48 (55 mg, 97% purity, 101%). LC-MS (Method 4): Rₜ=1.22 min; MS (ESIpos): m/z=972 [M+H]⁺.

### Example S60: Preparation of trifluoroacetic acid-benzyl N-methylglycyl-N-methylglycyl-N-methylglycyl-N-methylglycinate (1/1) (Intermediate 49)

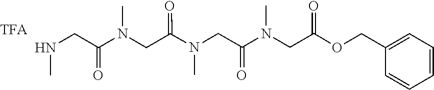 [SMILES: CNCC(=O)N(C)CC(=O)N(C)CC(=O)N(C)CC(=O)OCc1ccccc1]

To a solution of benzyl N-(tert-butoxycarbonyl)-N-methylglycyl-N-methylglycyl-N-methylglycyl-N-methylglycinate (488 mg, 97% purity, 965 μmol) (Intermediate 45) in DCM (20 ml), was added TFA (4.0 ml). The mixture was stirred at rt for 30 min and then concentrated under reduced pressure. The residue was dissolved in DCM and ether was added until the solution became cloudy. The supernatant was decanted and dried under vacuum to afford Intermediate 49 (490 mg, 100% purity, 100% yield). LC-MS (Method 4): Rₜ=0.70 min; MS (ESIpos): m/z=393 [M+H]+

### Example S61: Preparation of benzyl N-(tert-butoxycarbonyl)-N-methylglycyl-N-methylglycyl-N-methylglycyl-N-methylglycyl-N-methylglycyl-N-methylglycyl-N-methylglycyl-N-methylglycinate (Intermediate 50)

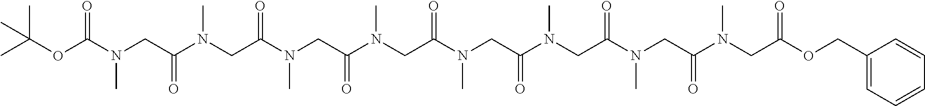 [SMILES: CN(CC(=O)OCc1ccccc1)C(=O)CN(C)C(=O)CN(C)C(=O)CN(C)C(=O)CN(C)C(=O)CN(C)C(=O)CN(C)C(=O)CN(C)C(=O)OC(C)(C)C]

To a solution of N-(tert-butoxycarbonyl)-N-methylglycyl-N-methylglycyl-N-methylglycyl-N-methylglycine (Intermediate 46) (175 mg, 434 μmol) in DMF (10 ml), were added trifluoroacetic acid-benzyl N-methylglycyl-N-methylglycyl-N-methylglycyl-N-methylglycinate (1/1) (200 mg, 395 μmol) (Intermediate 49) (63.2 mg, 129 μmol), HATU (195 mg, 513 μmol) and N,N-diisopropylethylamine (100 μl, 590 μmol). The mixture was stirred at rt for 1 h and then concentrated under reduced pressure. The residue was purified by preparative HPLC to afford benzyl N-(tert-butoxycarbonyl)-N-methylglycyl-N-methylglycyl-N-methylglycyl-N-methylglycyl-N-methylglycyl-N-methylglycyl-N-methylglycyl-N-methylglycinate (279 mg, 89% purity, 81% yield) as a colorless oil. LC-MS (Method 3): Rt=3.20 min; MS (ESIpos): m/z=777 [M+H]⁺.

### Example S62: Preparation of N-(tert-butoxycarbonyl)-N-methylglycyl-N-methylglycyl-N-methylglycyl-N-methylglycyl-N-methylglycyl-N-methylglycyl-N-methylglycyl-N-methylglycine (Intermediate 51)

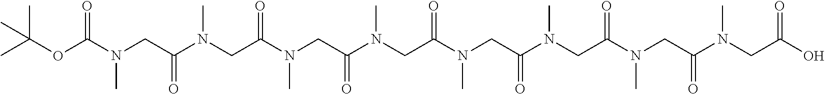 [SMILES: CN(CC(=O)O)C(=O)CN(C)C(=O)CN(C)C(=O)CN(C)C(=O)CN(C)C(=O)CN(C)C(=O)CN(C)C(=O)CN(C)C(=O)OC(C)(C)C]

Benzyl N-(tert-butoxycarbonyl)-N-methylglycyl-N-methylglycyl-N-methylglycyl-N-methylglycyl-N-methylglycyl-N-methylglycyl-N-methylglycyl-N-methylglycinate (140 mg, 180 μmol) (Intermediate 50) was dissolved in methanol (7 ml) and dichloromethane (7 ml). Pd/C 10% (13.8 mg) was added and the reaction was hydrogenated at RT for 2 h and filtered. The mother liquor was concentrated in vacuo, and then lyophilized to afford Intermediate 51 (110 mg, 80% purity, 71% yield) as a white amorphous residue. LC-MS (Method 3): Rₜ=2.12 min; MS (ESIpos): m/z=687 [M+H]⁺

### Example S63: Preparation of (2S)-1-[(30S)-30-(2-tert-butoxy-2-oxoethyl)-2,2,5,8,11,14,17,20,23,26-decamethyl-4,7,10,13,16,19,22,25,28,31-decaoxo-3-oxa-5,8,11,14,17,20,23,26,29-nonaazahentriacontan-31-yl]pyrrolidine-2-carboxylic acid (Intermediate 52)

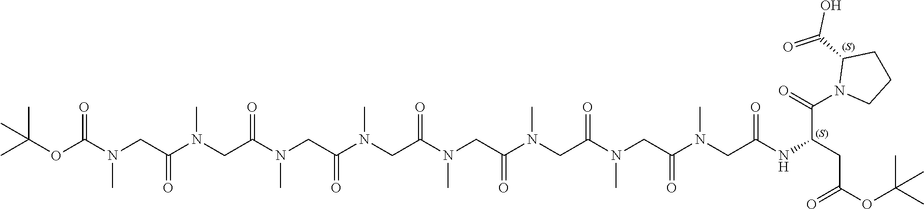

Step 1: To a solution of N-(tert-butoxycarbonyl)-N-methylglycyl-N-methylglycyl-N-methylglycyl-N-methylglycyl-N-methylglycyl-N-methylglycyl-N-methylglycyl-N-methylglycine (50.0 mg, 72.8 μmol) (Intermediate 51) in DMF (10 ml), were added trifluoroacetic acid-benzyl (2S)-1-[(2S)-2-amino-4-tert-butoxy-4-oxobutanoyl]pyrrolidine-2-carboxylate (1/1) (39.3 mg, 80.1 μmol) (Intermediate 43), HATU (41.5 mg, 109 μmol) and N,N-diisopropylethylamine (38 μl, 220 μmol). The mixture was stirred at rt for 10 min and then concentrated under reduced pressure. The residue was purified by preparative HPLC to afford benzyl (2S)-1-[(30S)-30-(2-tert-butoxy-2-oxoethyl)-2,2,5,8,11,14,17,20,23,26-decamethyl-4,7,10,13,16,19,22,25,28,31-decaoxo-3-oxa-5,8,11,14,17,20,23,26,29-nonaazahentriacontan-31-yl]pyrrolidine-2-carboxylate (58 mg, 98% purity, 75% yield) as an amorphous residue. LC-MS (Method 2): Rₜ=1.61 min; MS (ESIpos): m/z=1045 [M+H]⁺.
Step 2: Benzyl (2S)-1-[(30S)-30-(2-tert-butoxy-2-oxoethyl)-2,2,5,8,11,14,17,20,23,26-decamethyl-4,7,10,13,16,19,22,25,28,31-decaoxo-3-oxa-5,8,11,14,17,20,23,26,29-nonaazahentriacontan-31-yl]pyrrolidine-2-carboxylate (58.0 mg, 98% purity, 54.3 μmol) was dissolved in methanol (15 ml) and dichloromethane (5 ml). Pd/C 10% (10.0 mg) was added and the reaction was hydrogenated at RT for 1 h and filtered. The mother liquor was concentrated in vacuo, and then lyophilized to afford Intermediate 52 (49 mg, 93% purity, 88% yield) as an amorphous residue. LC-MS (Method 4): Rₜ=1.29 min; MS (ESIpos): m/z=955 [M+H]⁺.

### Example S64: Preparation of (4S)-4,11-diethyl-3,14-dioxo-3,4,12,14-tetrahydro-1H-pyrano[3′,4′:6,7]indolizino[1,2-b]quinolin-4-yl N-methylglycyl-N-methylglycyl-N-methylglycyl-N-methylglycyl-N-methylglycyl-N-methylglycyl-N-methylglycyl-N-methylglycyl-L-alpha-aspartyl-L-prolyl-L-valinate- trifluoroacetic acid (1/1) (Intermediate 53)

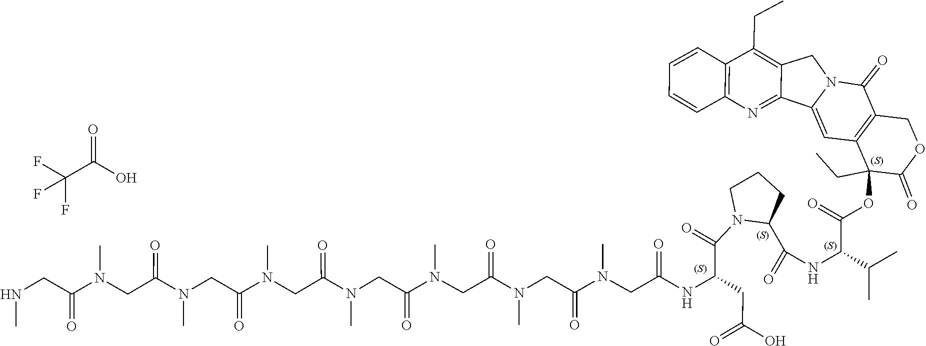

Step 1: To a solution of ((4S)-4,11-diethyl-3,14-dioxo-3,4,12,14-tetrahydro-1H-pyrano[3′,4′:6,7]indolizino[1,2-b]quinolin-4-yl N-[(5S)-5-amino-1-carboxy-4,11,15,28-tetraoxo-19,22,25-trioxa-3,10,16-triazaoctacosan-28-yl]-L-alpha-aspartyl-L-prolyl-L-valinate·trifluoroacetic acid (1/1) (Building Block 9) (31.0 mg, 52.5 μmol) in DMF (8 ml) were added (2S)-1-[(30S)-30-(2-tert-butoxy-2-oxoethyl)-2,2,5,8,11,14,17,20,23,26-decamethyl-4,7,10,13,16,19,22,25,28,31-decaoxo-3-oxa-5,8,11,14,17,20,23,26,29-nonaazahentriacontan-31-yl]pyrrolidine-2-carboxylic acid (49.0 mg, 93% purity, 47.8 μmol) (Intermediate 52), HATU (27.2 mg, 71.6 μmol) and DIEA (33 μl, 190 μmol). The mixture was stirred at rt for 10 min and then concentrated under reduced pressure. The residue was purified by preparative HPLC to afford tert-butyl (30S)-30-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2,5,8,11,14,17,20,23,26-decamethyl-4,7,10,13,16,19,22,25,28-nonaoxo-3-oxa-5,8,11,14,17,20,23,26,29-nonaazadotriacontan-32-oate (58 mg, 100% purity, 86% yield). LC-MS (Method 3): Rₜ=4.31 min; MS (ESIpos): m/z=1413 [M+H]⁺.
Step 2: To a solution of tert-butyl (30S)-30-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2,2,5,8,11,14,17,20,23,26-decamethyl-4,7,10,13,16,19,22,25,28-nonaoxo-3-oxa-5,8,11,14,17,20,23,26,29-nonaazadotriacontan-32-oate (58.0 mg, 41.1 μmol) in DCM (6 ml), was added TFA (4.0 ml). The mixture was stirred at rt for 2 h and then concentrated under reduced pressure. The residue was dissolved in ACN/H₂O and lyophilized to afford (4S)-4,11-diethyl-3,14-dioxo-3,4,12,14-tetrahydro-1H-pyrano[3′,4′:6,7]indolizino[1,2-b]quinolin-4-yl N-methylglycyl-N-methylglycyl-N-methylglycyl-N-methylglycyl-N-methylglycyl-N-methylglycyl-N-methylglycyl-N-methylglycyl-L-alpha- aspartyl-L-prolyl-L-valinate-trifluoroacetic acid (1/1) (Intermediate 53) (56 mg, 97% purity, 97%). LC-MS (Method 3): Rₜ=2.82 min; MS (ESIpos): m/z=1257 [M+H]⁺.

### Example S65: Preparation of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2-(tert-butoxycarbonyl)-L-lysyl]amino}ethoxy)ethoxy]ethoxy}propanoyl)-L-alpha-aspartyl-L-prolyl-L-valinate (Intermediate 54)

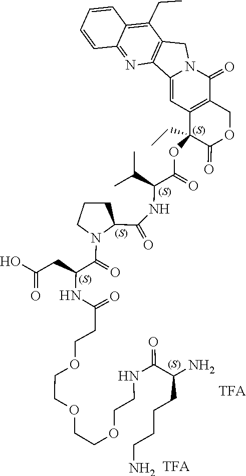

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-valinate-trifluoroacetic acid (1/1) (150 mg, 149 μmol) (Intermediate 6) in DMF (10 ml), was added 2,5-dioxopyrrolidin-1-yl N⁶-[(benzyloxy)carbonyl]-N²-(tert-butoxycarbonyl)-L-lysinate (92.7 mg, 194 μmol) and N,N-diisopropylethylamine (52 μl, 300 μmol). The mixture was stirred at rt for 1 h and then concentrated under reduced pressure. The residue was purified by preparative HPLC to afford (4S)-4,11-diethyl-3,14-dioxo-3,4,12,14-tetrahydro-1H-pyrano[3′,4′:6,7]indolizino[1,2-b]quinolin-4-yl N-[3-(2-{2-[2-({N6-[(benzyloxy)carbonyl]-N2-(tert-butoxycarbonyl)-L-lysyl}amino)ethoxy]ethoxy}ethoxy)propanoyl]-L-alpha-aspartyl-L-prolyl-L-valinate (170 mg, 87% purity, 79% yield) as a yellow foam. LC-MS (Method 3): Rₜ=4.62 min; MS (ESIpos): m/z=1254 [M+H]⁺.
Step 2: (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-({N6-[(benzyloxy)carbonyl]-N2-(tert-butoxycarbonyl)-L-lysyl}amino)ethoxy]ethoxy}ethoxy)propanoyl]-L-alpha-aspartyl-L-prolyl-L-valinate (170 mg, 136 μmol) was dissolved in methanol (20 ml) and dichloromethane (20 ml). Pd/C 10% (25 mg) was added and the reaction was hydrogenated at RT for 2 h and filtered. The mother liquor was concentrated under reduced pressure, dissolved in acetonitrile/water and freeze dried to afford Intermediate 54 (145 mg, 73% purity, 70% yield) as a yellow foam. LC-MS (Method 3): Rₜ=3.18 min; MS (ESIpos): m/z=1120 [M+H]⁺.

### Example S66: Preparation of N-{5-[(2,5-dioxopyrrolidin-1-yl)oxy]-5-oxopentanoyl}-N-methylglycyl-N-methylglycyl-N-methylglycyl-N-methylglycine (Intermediate 55)

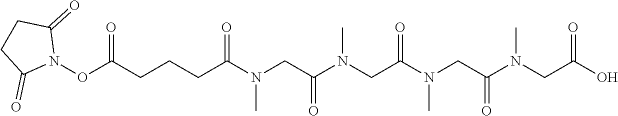 [SMILES: CN(CC(=O)O)C(=O)CN(C)C(=O)CN(C)C(=O)CN(C)C(=O)CCCC(=O)ON1C(=O)CCC1=O]

Step 1. Trifluoroacetic acid-benzyl N-methylglycyl-N-methylglycyl-N-methylglycyl-N-methylglycinate (1/1) (Intermediate 49) (100 mg, 197 μmol) was dissolved in methanol (4 ml) and dichloromethane (4 ml). Pd/C 10% (10 mg) was added and the reaction was hydrogenated at rt for 2 h and filtered. The mother liquor was concentrated under reduced pressure, dissolved in acetonitrile/water and freeze dried to afford N-methylglycyl-N-methylglycyl-N-methylglycyl-N-methylglycine-trifluoroacetic acid (1/1) (90 mg, 100% purity, 109% yield) as a colorless foam. LC-MS (Method 2): Rₜ=0.20 min; MS (ESIpos): m/z=303 [M+H]⁺.
Step 2: To a solution of N-methylglycyl-N-methylglycyl-N-methylglycyl-N-methylglycine-trifluoroacetic acid (1/1) (90.0 mg, 216 μmol) in DMF (10 ml), were added 1,1′-[(1,5-dioxopentane-1,5-diyl)bis(oxy)]di(pyrrolidine-2,5-dione) (141 mg, 432 μmol) and N,N-diisopropylethylamine (110 μl, 650 μmol). The mixture was stirred at rt for 30 min and then concentrated under reduced pressure. The residue was purified by preparative HPLC to afford Intermediate 55 (24 mg, 93% purity, 20% yield) as a colorless foam. LC-MS (Method 3): Rt=1.55 min; MS (ESIpos): m/z=514 [M+H]⁺.

### Example S67: Preparation of (23S,39S)-23-amino-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid-trifluoroacetic acid (1/1) (Intermediate 56)

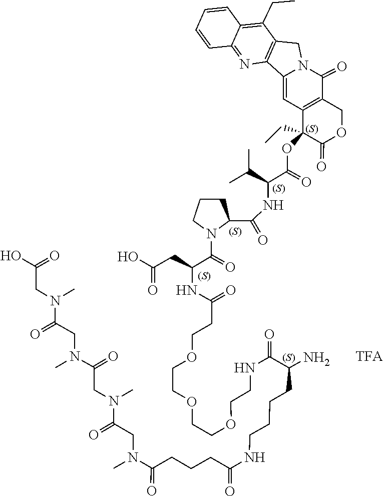

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2-(tert-butoxycarbonyl)-L-lysyl]amino}ethoxy)ethoxy]ethoxy}propanoyl)-L-alpha-aspartyl-L-prolyl-L-valinate (43.6 mg, 38.9 μmol) (Intermediate 54) in DMF (8 ml) were added N-{5-[(2,5-dioxopyrrolidin-1-yl)oxy]-5-oxopentanoyl}-N-methylglycyl-N-methylglycyl-N-methylglycyl-N-methylglycine (24.0 mg, 46.7 μmol) (Intermediate 55) and DIEA (54 μl, 310 μmol). The mixture was stirred at rt for 2 h and then concentrated under reduced pressure. The residue was purified by preparative HPLC to afford (23S,39S)-23-[(tert-butoxycarbonyl)amino]-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid (29 mg, 93% purity, 45% yield) as a yellow foam. LC-MS (Method 3): Rₜ=3.73 min; MS (ESIpos): m/z=1517 [M+H]⁺.
Step 2: To a solution of (23S,39S)-23-[(tert-butoxycarbonyl)amino]-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid (29.0 mg, 19.1 μmol) in DCM (5 ml), was added TFA (1.0 ml). The mixture was stirred at rt for 1 h and then concentrated under reduced pressure. The residue was dissolved in ACN/H₂O and lyophilized to afford Intermediate 56 (32 mg, 100% purity, 109%) as a yellow foam. LC-MS (Method 3): Rₜ=2.96 min; MS (ESIpos): m/z=1417 [M+H]⁺.

### Example S68: Preparation of (23S,39S)-23-(3-{2-[2-(2-aminoethoxy)ethoxy]ethoxy}propanamido)-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid-trifluoroacetic acid (1/1) (Intermediate 57)

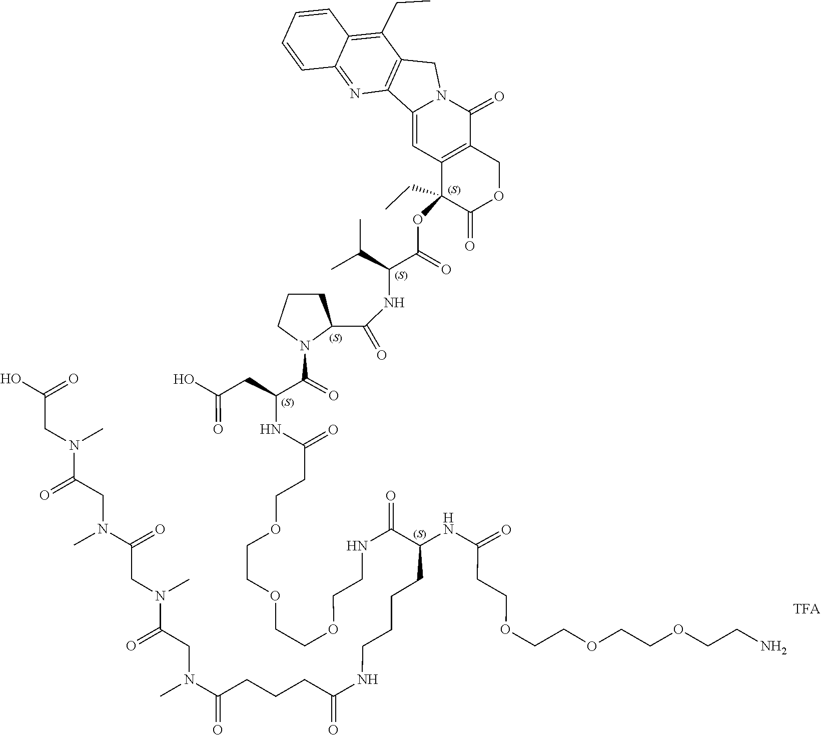

Step 1: To a solution of (23S,39S)-23-amino-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid-trifluoroacetic acid (1/1) (32.0 mg, 20.9 μmol) (Intermediate 56) in DMF (5 ml) was added tert-butyl {2-[2-(2-{3-[(2,5-dioxopyrrolidin-1-yl)oxy]-3-oxopropoxy}ethoxy)ethoxy]ethyl}carbamate (21.0 mg, 50.1 μmol) (Intermediate 18) and DIEA (29 μl, 170 μmol). The mixture was stirred at rt for 2 h and then concentrated under reduced pressure. The residue was purified by preparative HPLC to afford (23S,39S)-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-23-[(2,2-dimethyl-4,17-dioxo-3,8,11,14-tetraoxa-5-azaheptadecan-17-yl)amino]-3,6,9,12-tetramethyl-4,7,10,13,17,24,37-heptaoxo-28,31,34-trioxa-3,6,9,12,18,25,38-heptaazahentetracontane-1,41-dioic acid (44 mg, 88% purity, 108% yield) as a yellow foam. LC-MS (Method): Rₜ=3.84 min; MS (ESIpos): m/z=1720 [M+H]⁺.
Step 2: To a solution of (23S,39S)-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-23-[(2,2-dimethyl-4,17-dioxo-3,8,11,14-tetraoxa-5-azaheptadecan-17-yl)amino]-3,6,9,12-tetramethyl-4,7,10,13,17,24,37-heptaoxo-28,31,34-trioxa-3,6,9,12,18,25,38-heptaazahentetracontane-1,41-dioic acid (44.0 mg, 88% purity, 22.5 μmol) in DCM (10 ml), was added TFA (2.0 ml). The mixture was stirred at rt for 1 h and then concentrated under reduced pressure. The residue was dissolved in ACN/H₂O and lyophilized to afford Intermediate 57 (46 mg, 87% purity, 102%) as a yellow foam. LC-MS (Method 3): Rₜ=2.95 min; MS (ESIpos): m/z=1620 [M+H]⁺

### Example S69: Preparation of benzyl N-{5-[(2,5-dioxopyrrolidin-1-yl)oxy]-5-oxopentanoyl}-N-methylglycyl-N-methylglycyl-N-methylglycyl-N-methylglycyl-N-methylglycyl-N-methylglycyl-N-methylglycyl-N-methylglycinate (Intermediate 58)

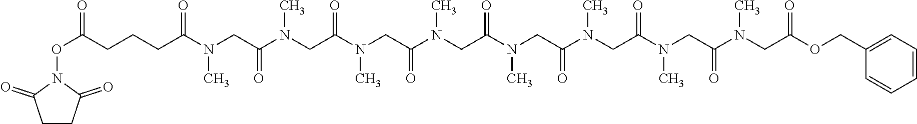 [SMILES: CN(CC(=O)OCc1ccccc1)C(=O)CN(C)C(=O)CN(C)C(=O)CN(C)C(=O)CN(C)C(=O)CN(C)C(=O)CN(C)C(=O)CN(C)C(=O)CCCC(=O)ON1C(=O)CCC1=O]

Step 1: To a solution of benzyl N-(tert-butoxycarbonyl)-N-methylglycyl-N-methylglycyl-N-methylglycyl-N-methylglycyl-N-methylglycyl-N-methylglycyl-N-methylglycyl-N-methylglycinate (139 mg, 179 μmol) (Intermediate 50): in DCM (20 ml), was added TFA (4.0 ml). The mixture was stirred at rt for 1 h and then concentrated under reduced pressure. The residue was dissolved in ACN/H₂O and lyophilized to afford trifluoroacetic acid-benzyl N-methylglycyl-N-methylglycyl-N-methylglycyl-N-methylglycyl-N-methylglycyl-N-methylglycyl-N-methylglycyl-N-methylglycinate (1/1) (160 mg, 86% purity, 97%) as a colorless foam. LC-MS (Method 3): Rₜ=1.83 min; MS (ESIpos): m/z=677 [M+H]⁺.
Step 2: To a solution of trifluoroacetic acid-benzyl N-methylglycyl-N-methylglycyl-N-methylglycyl-N-methylglycyl-N-methylglycyl-N-methylglycyl-N-methylglycyl-N-methylglycinate (1/1) (80.0 mg, 85% purity, 86.0 μmol) in DMF (8.7 ml), were added 1,1′-[(1,5-dioxopentane-1,5-diyl)bis(oxy)]di(pyrrolidine-2,5-dione) (84.2 mg, 258 μmol) and N,N-diisopropylethylamine (45 μl, 260 μmol). The mixture was stirred at rt for 30 min and then concentrated under reduced pressure. The residue was purified by preparative HPLC to afford Intermediate 58 (52 mg, 91% purity, 62% yield) as a colorless foam. LC-MS (Method 3): Rt=2.70 min; MS (ESIpos): m/z=888 [M+H]⁺.

### Example S70: Preparation of (37S,53S)-37-amino-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid-trifluoroacetic acid (1/1) (Intermediate 59)

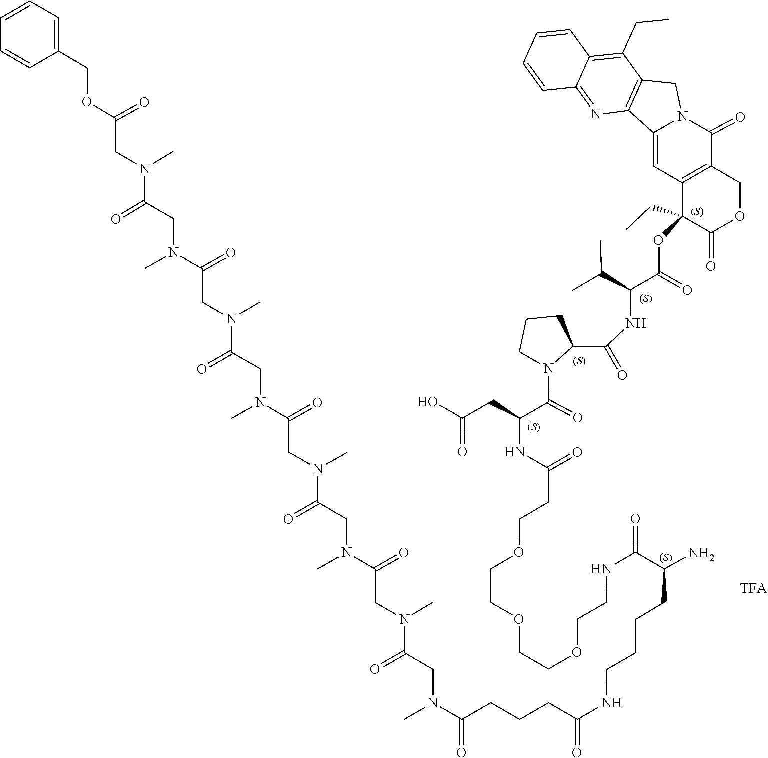

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2-(tert-butoxycarbonyl)-L-lysyl]amino}ethoxy)ethoxy]ethoxy}propanoyl)-L-alpha-aspartyl-L-prolyl-L-valinate (20.0 mg, 17.9 μmol) (Intermediate 54) in DMF (4 ml) were added benzyl N-{5-[(2,5-dioxopyrrolidin-1-yl)oxy]-5-oxopentanoyl}-N-methylglycyl-N-methylglycyl-N-methylglycyl-N-methylglycyl-N-methylglycyl-N-methylglycyl-N-methylglycyl-N-methylglycinate (25.4 mg, 28.6 μmol) (Intermediate 58) and DIEA (25 μl, 140 μmol). The mixture was stirred at rt for 3 h and then concentrated under reduced pressure. The residue was purified by preparative HPLC to afford (37S,53S)-37-[(tert-butoxycarbonyl)amino]-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid (25.8 mg, 95% purity, 72% yield) as a yellow foam. LC-MS (Method 3): Rₜ=4.02 min; MS (ESIpos): m/z=1891 [M+H]⁺
Step 2: To a solution of (37S,53S)-37-[(tert-butoxycarbonyl)amino]-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid (25.8 mg, 13.6 μmol) in DCM (5 ml), was added TFA (1.0 ml). The mixture was stirred at rt for 1 h and then concentrated under reduced pressure. The residue was dissolved in ACN/H₂O and lyophilized to afford Intermediate 59 (19.5 mg, 92% purity, 69%) as a yellow foam. LC-MS (Method 3): Rₜ=3.29 min; MS (ESIpos): m/z=1791 [M+H]⁺.

### Example S71: Preparation of (35S,51S)-35-(3-{2-[2-(2-aminoethoxy)ethoxy]ethoxy}propanamido)-51-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12,15,18,21,24-octamethyl-4,7,10,13,16,19,22,25,29,36,49-undecaoxo-40,43,46-trioxa-3,6,9,12,15,18,21,24,30,37,50-undecaazatripentacontane-1,53-dioic acid-trifluoroacetic acid (1/1) (Intermediate 60)

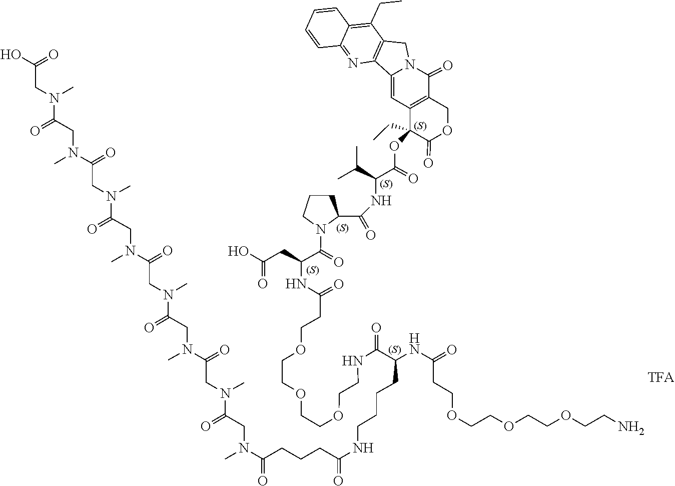

Step 1: To a solution of (37S,53S)-37-amino-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid-trifluoroacetic acid (1/1) (19.5 mg, 10.2 μmol) (Intermediate 59) in DMF (4 ml) was added tert-butyl {2-[2-(2-{3-[(2,5-dioxopyrrolidin-1-yl)oxy]-3-oxopropoxy}ethoxy)ethoxy]ethyl}carbamate (10.3 mg, 24.6 μmol) (Intermediate 18) and DIEA (14 μl, 82 μmol). The mixture was stirred at rt for 2 h and then concentrated under reduced pressure. The residue was purified by preparative HPLC to afford (375,53S)-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-37-[(2,2-dimethyl-4,17-dioxo-3,8,11,14-tetraoxa-5-azaheptadecan-17-yl)amino]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid (27 mg, 82% purity, 103% yield) as a yellow foam. LC-MS (Method 3): Rₜ=4.11 min; MS (ESIpos): m/z=2095 [M+H]⁺.
Step 2: To a solution of (37S,53S)-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-37-[(2,2-dimethyl-4,17-dioxo-3,8,11,14-tetraoxa-5-azaheptadecan-17-yl)amino]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid (27.0 mg, 12.9 μmol) in DCM (4 ml), was added TFA (1.0 ml). The mixture was stirred at rt for 1 h and then concentrated under reduced pressure. The residue was dissolved in ACN/H₂O and lyophilized to afford (37S,53S)-37-(3-{2-[2-(2-aminoethoxy)ethoxy]ethoxy}propanamido)-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid-trifluoroacetic acid (1/1) (28 mg, 85% purity, 88%) as a yellow foam. LC-MS (Method 3): Rt=3.27 min; MS (ESIpos): m/z=1994 [M+H]⁺.
Step 3: (37S,53S)-37-(3-{2-[2-(2-aminoethoxy)ethoxy]ethoxy}propanamido)-53-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-5,8,11,14,17,20,23,26-octamethyl-3,6,9,12,15,18,21,24,27,31,38,51-dodecaoxo-1-phenyl-2,42,45,48-tetraoxa-5,8,11,14,17,20,23,26,32,39,52-undecaazapentapentacontan-55-oic acid-trifluoroacetic acid (1/1) (28.0 mg, 13.3 μmol) was dissolved in methanol (2.5 ml) and dichloromethane (2.5 ml). Pd/C 10% (5 mg) was added and the reaction was hydrogenated at rt for 2 h and filtered. The mother liquor was concentrated under reduced pressure to afford Intermediate 60 (27 mg, 91% purity, 92% yield) as a colorless foam. LC-MS (Method 3): Rₜ=2.94 min; MS (ESIpos): m/z=1904 [M+H]⁺.

### Example S72: Preparation of benzyl N-{5-[(2,5-dioxopyrrolidin-1-yl)oxy]-5-oxopentanoyl}-N-methylglycyl-N-methylglycyl-N-methylglycyl-N-methylglycyl-N-methylglycyl-N-methylglycyl-N-methylglycyl-N-methylglycinate (Intermediate 61)

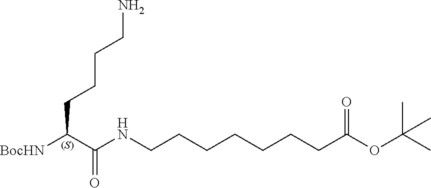

Step 1: To a solution of N⁶-[(benzyloxy)carbonyl]-N²-(tert-butoxycarbonyl)-L-lysine (600 mg, 1.58 mmol) in DMF (15 ml), were added tert-butyl 8-aminooctanoate (374 mg, 1.73 mmol), HATU (780 mg, 2.05 mmol) and N,N-diisopropylethylamine (410 μl, 2.4 mmol). The mixture was stirred at rt for 1 h. Ethyl acetate was then added and the organic layer was washed with water and brine. The organic phase was then dried over sodium sulphate and concentrated under reduced pressure. The residue was purified by preparative HPLC and lyophilized to afford tert-butyl 8-({N6-[(benzyloxy)carbonyl]-N2-(tert-butoxycarbonyl)-L-lysyl}amino)octanoate (940 mg, 89% purity, 91% yield) as an amorphous residue. LC-MS (Method 2): Rₜ=2.33 min; MS (ESIpos): m/z=578 [M+H]⁺.
Step 2: Tert-butyl 8-({N6-[(benzyloxy)carbonyl]-N2-(tert-butoxycarbonyl)-L-lysyl}amino)octanoate (938 mg, 1.62 mmol) was dissolved in methanol (25 ml) and dichloromethane (25 ml). Pd/C 10% (125 mg) was added and the reaction was hydrogenated at RT for 5 h and filtered over celite. The mother liquor was concentrated under reduced pressure to afford Intermediate 61 (674 mg, 91% purity, 85% yield) as a colorless resin. LC-MS (Method 4): Rₜ=1.41 min; MS (ESIpos): m/z=444 [M+H]⁺.

### Example S73: Preparation of (4S)-4,11-diethyl-3,14-dioxo-3,4,12,14-tetrahydro-1H-pyrano[3′,4′:6,7]indolizino[1,2-b]quinolin-4-yl N-[(24S)-24-amino-33-carboxy-14,18,25-trioxo-4,7,10-trioxa-13,19,26-triazatritriacontanan-1-oyl]-L-alpha-aspartyl-L-prolyl-L-valinate-trifluoroacetic acid (½) (Intermediate 62)

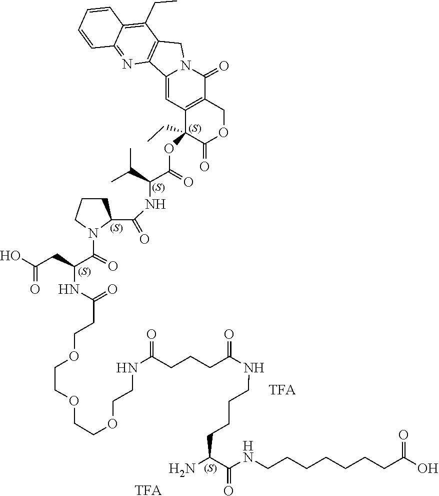

Step 1: To a solution of (4S)-4,11-diethyl-3,14-dioxo-3,4,12,14-tetrahydro-1H-pyrano[3′,4′:6,7]indolizino[1,2-b]quinolin-4-yl N-{18-[(2,5-dioxopyrrolidin-1-yl)oxy]-14,18-dioxo-4,7,10-trioxa-13-azaoctadecanan-1-oyl}-L-alpha-aspartyl-L-prolyl-L-valinate (65.0 mg, 93% purity, 54.6 μmol) (Intermediate 39) in DMF (2.5 ml) were added tert-butyl 8-{[N²-(tert-butoxycarbonyl)-L-lysyl]amino}octanoate (39.8 mg, 91% purity, 82.0 μmol) (Intermediate 61) and DIEA (38 μl, 220 μmol). The mixture was stirred at rt for 2 h and then concentrated under reduced pressure. The residue was purified by preparative HPLC and lyophilized to afford (4S)-4,11-diethyl-3,14-dioxo-3,4,12,14-tetrahydro-TH-pyrano[3′,4′:6,7]indolizino[1,2-b]quinolin-4-yl N-{(14S)-14-[(tert-butoxycarbonyl)amino]-2,2-dimethyl-4,13,20,24,37-pentaoxo-3,28,31,34-tetraoxa-12,19,25-triazaheptatriacontan-37-yl}-L-alpha-aspartyl-L-prolyl-L-valinate (66 mg, 92% purity, 78% yield) as a yellow foam. LC-MS (Method 3): Rₜ=4.92 min; MS (ESIpos): m/z=1431 [M+H]r
Step 2: To a solution of (4S)-4,11-diethyl-3,14-dioxo-3,4,12,14-tetrahydro-1H-pyrano[3′,4′:6,7]indolizino[1,2-b]quinolin-4-yl N-{(14S)-14-[(tert-butoxycarbonyl)amino]-2,2-dimethyl-4,13,20,24,37-pentaoxo-3,28,31,34-tetraoxa-12,19,25-triazaheptatriacontan-37-yl}-L-alpha-aspartyl-L-prolyl-L-valinate (65.0 mg, 92% purity, 41.8 μmol) in DCM (2.5 ml), was added TFA (1.2 ml). The mixture was stirred at rt for 1 h and then concentrated under reduced pressure. The residue was dissolved in ACN/H₂O and lyophilized to afford Intermediate 62 (78 mg, 94% purity, 117%) as an amorphous residue. LC-MS (Method 3): Rₜ=3.08 min; MS (ESIpos): m/z=1275 [M+H]⁺.

### Example S74: Preparation of (4S)-4,11-diethyl-3,14-dioxo-3,4,12,14-tetrahydro-1H-pyrano[3′,4′:6,7]indolizino[1,2-b]quinolin-4-yl N-{(24S)-37-amino-24-[(7-carboxyheptyl)carbamoyl]-14,18,26-trioxo-4,7,10,29,32,35-hexaoxa-13,19,25-triazaheptatriacontanan-1-oyl}-L-alpha-aspartyl-L-prolyl-L-valinate-trifluoroacetic acid (½) (Intermediate 63)

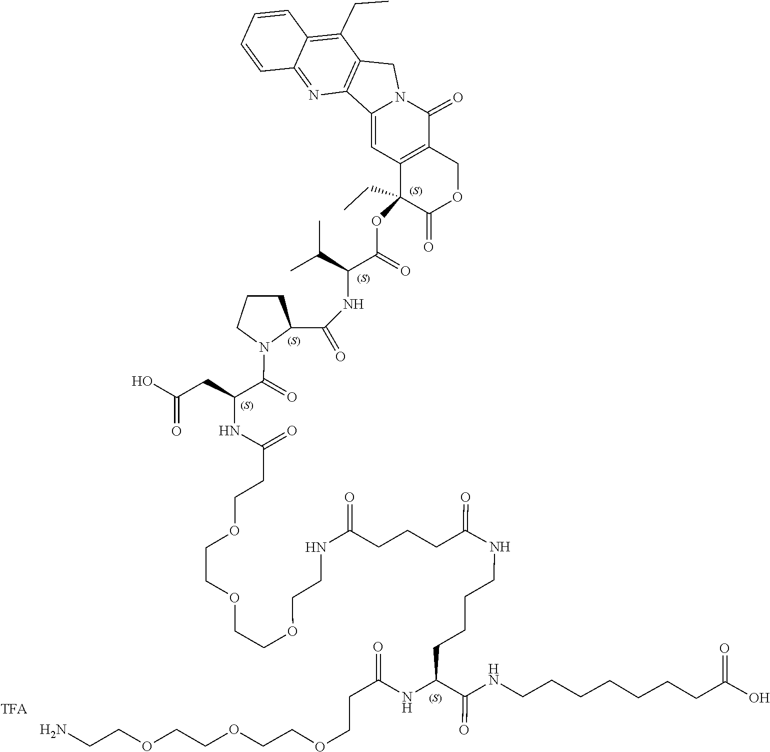

Step 1: To a solution of (4S)-4,11-diethyl-3,14-dioxo-3,4,12,14-tetrahydro-1H-pyrano[3′,4′:6,7]indolizino[1,2-b]quinolin-4-yl N-[(24S)-24-amino-33-carboxy-14,18,25-trioxo-4,7,10-trioxa-13,19,26-triazatritriacontanan-1-oyl]-L-alpha-aspartyl-L-prolyl-L-valinate-trifluoroacetic acid (½) (75.0 mg, 94% purity, 47.1 μmol) (Intermediate 62) in DMF (5 ml) was added tert-butyl {2-[2-(2-{3-[(2,5-dioxopyrrolidin-1-yl)oxy]-3-oxopropoxy}ethoxy)ethoxy]ethyl}carbamate (47.3 mg, 113 μmol) (Intermediate 18) and DIEA (66 μl, 380 μmol). The mixture was stirred at rt for 1 h and then concentrated under reduced pressure. The residue was purified by preparative HPLC and lyophilized to afford (4S)-4,11-diethyl-3,14-dioxo-3,4,12,14-tetrahydro-1H-pyrano[3′,4′:6,7]indolizino[1,2-b]quinolin-4-yl N-{(19S)-19-[(7-carboxyheptyl)carbamoyl]-2,2-dimethyl-4,17,25,29,42-pentaoxo-3,8,11,14,33,36,39-heptaoxa-5,18,24,30-tetraazadotetracontan-42-yl}-L-alpha-aspartyl-L-prolyl-L-valinate (77 mg, 90% purity, 93% yield) as an amorphous residue. LC-MS (Method 3): Rt=4.10 min; MS (ESIpos): m/z=1578 [M+H]⁺.
Step 2: To a solution of (4S)-4,11-diethyl-3,14-dioxo-3,4,12,14-tetrahydro-1H-pyrano[3′,4′:6,7]indolizino[1,2-b]quinolin-4-yl N-{(19S)-19-[(7-carboxyheptyl)carbamoyl]-2,2-dimethyl-4,17,25,29,42-pentaoxo-3,8,11,14,33,36,39-heptaoxa-5,18,24,30-tetraazadotetracontan-42-yl}-L-alpha-aspartyl-L-prolyl-L-valinate (76.0 mg, 90% purity, 43.4 μmol) in DCM (2.5 ml), was added TFA (1.2 ml). The mixture was stirred at rt for 1 h and then concentrated under reduced pressure. The residue was dissolved in ACN/H₂O and lyophilized to afford Intermediate 63 (88 mg, 90% purity, 107%) as an amorphous residue. LC-MS (Method 3): Rₜ=3.10 min; MS (ESIpos): m/z=1478 [M+H]⁺.

### Example S75: Preparation of trifluoroacetic acid-(4S)-4,11-diethyl-3,14-dioxo-3,4,12,14-tetrahydro-1H-pyrano[3′,4′:6,7]indolizino[1,2-b]quinolin-4-yl L-alaninate (1/1) (Intermediate 64)

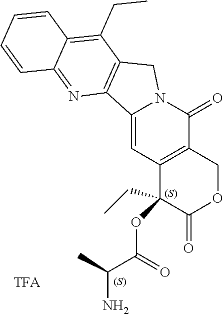

Step 1: To a solution of (4S)-4,11-diethyl-4-hydroxy-1H-pyrano[3′,4′:6,7]indolizino[1,2-b]quinoline-3,14(4H,12H)-dione (100 mg, 266 μmol) in DCM (20 ml) was added tert-butyl (4S)-4-methyl-2,5-dioxo-1,3-oxazolidine-3-carboxylate (137 mg, 638 μmol) and DMAP (58.4 mg, 478 μmol). The mixture was refluxed for 4 h and then concentrated under reduced pressure. The residue was purified by preparative HPLC and lyophilized to afford (4S)-4,11-diethyl-3,14-dioxo-3,4,12,14-tetrahydro-1H-pyrano[3′,4′:6,7]indolizino[1,2-b]quinolin-4-yl N-(tert-butoxycarbonyl)-L-alaninate (132 mg, 95% purity, 86% yield) as a yellow foam. LC-MS (Method 2): Rₜ=2.06 min; MS (ESIpos): m/z=548 [M+H]⁺.
Step 2: To a solution of (4S)-4,11-diethyl-3,14-dioxo-3,4,12,14-tetrahydro-1H-pyrano[3′,4′:6,7]indolizino[1,2-b]quinolin-4-yl N-(tert-butoxycarbonyl)-L-alaninate (131 mg, 95% purity, 227 μmol) in DCM (10 ml), was added TFA (2.0 ml). The mixture was stirred at rt for 1 h and then concentrated under reduced pressure. The residue was dissolved in ACN/H₂O and lyophilized to afford Intermediate 64 (156 mg, 100% purity, 122%) as an amorphous residue. LC-MS (Method 2): Rₜ=1.08 min; MS (ESIpos): m/z=448 [M+H]⁺.

### Example S76: Preparation of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-alaninate-trifluoroacetic acid (1/1) (Intermediate 65)

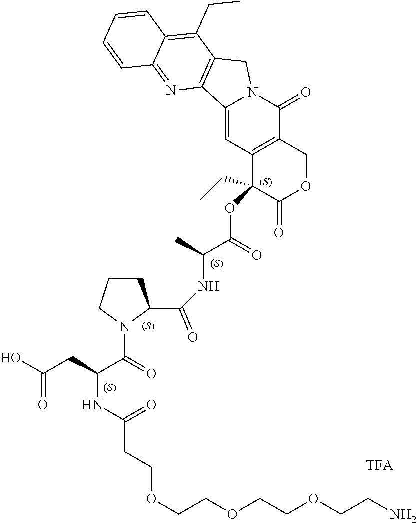

Step 1: To a solution of trifluoroacetic acid-(4S)-4,11-diethyl-3,14-dioxo-3,4,12,14-tetrahydro-1H-pyrano[3′,4′:6,7]indolizino[1,2-b]quinolin-4-yl L-alaninate (1/1) (156 mg, 100% purity, 278 μmol) (Intermediate 64) in DMF (15 ml) was added (2S)-1-[(19S)-19-(2-tert-butoxy-2-oxoethyl)-2,2-dimethyl-4,17,20-trioxo-3,8,11,14-tetraoxa-5,18-diazaicosan-20-yl]pyrrolidine-2-carboxylic acid (196 mg, 333 μmol) (Building Block 3), HATU (137 mg, 361 μmol) and DIEA (150 μl, 830 μmol). The mixture was stirred at rt for 1 h and then concentrated under reduced pressure. The residue was purified by preparative HPLC and lyophilized to afford tert-butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-1-oxopropan-2-yl]carbamoyl}pyrrolidine-1-carbonyl]-2,2-dimethyl-4,17-dioxo-3,8,11,14-tetraoxa-5,18-diazahenicosan-21-oate (220 mg, 100% purity, 78% yield) as an amorphous residue. LC-MS (Method 2): Rₜ=1.99 min; MS (ESIpos): m/z=1019 [M+H]⁺.
Step 2: To a solution of tert-butyl (19S)-19-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-1-oxopropan-2-yl]carbamoyl}pyrrolidine-1-carbonyl]-2,2-dimethyl-4,17-dioxo-3,8,11,14-tetraoxa-5,18-diazahenicosan-21-oate (219 mg, 100% purity, 215 μmol) in DCM (40 ml), was added TFA (10 ml). The mixture was stirred at rt for 3 h and then concentrated under reduced pressure. The residue was dissolved in ACN/H₂O and lyophilized to afford Intermediate 65 (240 mg, 94% purity, 108%) as an amorphous residue. LC-MS (Method 2): Rₜ=1.16 min; MS (ESIneg): m/z=861 [M−H]+.

### Example S77: Preparation of (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-alaninate·trifluoroacetic acid (1:1) (Intermediate 66)

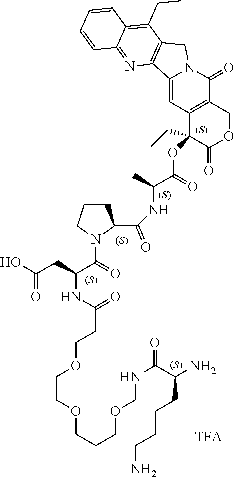

Step 1: (4S)-4,11-Diethyl-3,14-dioxo-3,4,12,14-tetrahydro-1H-pyrano[3′,4′:6,7]indolizino[1,2-b]quinolin-4-yl N-(2,2-dimethyl-4,17-dioxo-3,8,11,14-tetraoxa-5-azaheptadecan-17-yl)-L-alpha-aspartyl-L-prolyl-L-alaninate-trifluoroacetic acid (Intermediate 65) (50.0 mg, 94% purity, 48.1 μmol) was dissolved in 8 mL DMF and 2,5-dioxopyrrolidin-1-yl N²,N⁶-bis(tert-butoxycarbonyl)-L-lysinate (27.7 mg, 62.5 μmol) as well as N,N-diisopropylethylamine (34 μl, 190 μmol) were added. After stirring for 2 h at rt the mixture was concentrated in vacuo and the residual was purified by prep. HPLC. Relevant fractions were collected and concentrated in vacuo to yield (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2,N6-bis(tert-butoxycarbonyl)-L-lysyl]amino}ethoxy)ethoxy]ethoxy}propanoyl)-L-alpha-aspartyl-L-prolyl-L-alaninate (35.2 mg, 100% purity, 61% yield). LC-MS (Method 3): Rₜ=4.33 min; MS (ESIpos): m/z=1192 [M+H]⁺.
Step 2: (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2,N6-bis(tert-butoxycarbonyl)-L-lysyl]amino}ethoxy)ethoxy]ethoxy}propanoyl)-L-alpha-aspartyl-L-prolyl-L-alaninate (34.7 mg, 100% purity, 29.1 μmol) was dissolved in 4 ml DCM and 1 ml TFA was added and the reaction mixture was stirred for 1 h at rt. It was concentrated in vacuo, the residue was dissolved in ACN/H₂O and lyophilized to give Intermediate 66 (34.9 mg, 100% purity, 98% yield) as an amorphous residue. LC-MS (Method 2): Rₜ=0.93 min; MS (ESIneg): m/z=989 [M−H]⁻.

### Example S78: Preparation of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N²,N⁶-bis(3-{2-[2-(2-aminoethoxy)ethoxy]ethoxy}propanoyl)-L-lysyl]amino}ethoxy)ethoxy]ethoxy}propanoyl)-L-alpha-aspartyl-L-prolyl-L-alaninate·trifluoroacetic acid (½) (Intermediate 67)

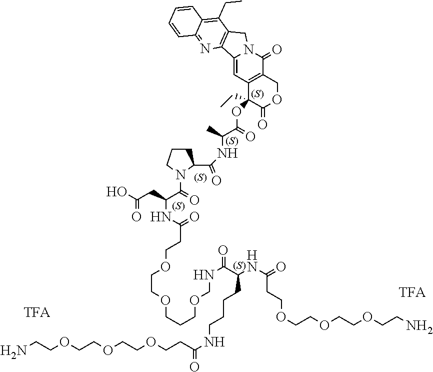

Step 1: (4S)-4,11-Diethyl-3,14-dioxo-3,4,12,14-tetrahydro-1H-pyrano[3′,4′:6,7]indolizino[1,2-b]quinolin-4-yl N-[3-(2-{2-[2-(L-lysylamino)ethoxy]ethoxy}ethoxy)propanoyl]-L-alpha-aspartyl-L-prolyl-L-alaninate (Intermediate 66) (34.2 mg, 100% purity, 28.1 μmol) was dissolved in 5 mL DMF and tert-butyl {2-[2-(2-{3-[(2,5-dioxopyrrolidin-1-yl)oxy]-3-oxopropoxy}ethoxy)ethoxy]ethyl}carbamate (28.2 mg, 67.3 μmol) (Intermediate 18) as well as N,N-diisopropylethylamine (39 μl, 220 μmol) were added. After stirring for 2 h at rt the mixture was concentrated in vacuo and the residual was purified by prep. HPLC. Relevant fractions were collected and concentrated in vacuo to yield (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2,N6-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-L-alaninate as an amorphous residue (31 mg, 100% purity, 69% yield). LC-MS (Method 3): Rₜ=4.18 min; MS (ESIpos): m/z=1598 [M+H]f.
Step 2: (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-{[N2,N6-bis(2,2-dimethyl-4,17-dioxo-3,8,11,14-tetraoxa-5-azaheptadecan-17-yl)-L-lysyl]amino}ethoxy)ethoxy]ethoxy}propanoyl)-L-alpha-aspartyl-L-prolyl-L-alaninate (29.1 mg, 100% purity, 18.2 μmol) was dissolved in 4 ml DCM and 1 ml TFA was added and the reaction mixture was stirred for 1 h at rt. It was concentrated in vacuo, the residue was dissolved in ACN/H₂O and lyophilized to give Intermediate 67 as an amorphous residue. (28.6 mg, 100% purity, 97% yield). LC-MS (Method 3): Rₜ=2.40 min; MS (ESIpos): m/z=1398 [M+H]7.

### Compounds

### Example A1: Preparation of Trisodium (4S)-4,11-diethyl-3,14-dioxo-3,4,12,14-tetrahydro-1H-pyrano[3′,4′:6,7]indolizino [1,2-b]quinoline-4-yl 1-[(2S)-15-{[N²,N⁶-bis({4-[({(1R)-2-carboxylato-1-[3-({3-[(propylcarbamoyl)amino]benzene-1-sulfonyl}amino)phenyl]ethyl}carbamoyl)amino]phenyl} carbamoyl)-L-lysyl]amino}-2-(carboxylatomethyl)-4-oxo-7,10,13-trioxa-3-azapentadecanan-1-oyl]-L-prolyl-L-valinate (Compound A1)

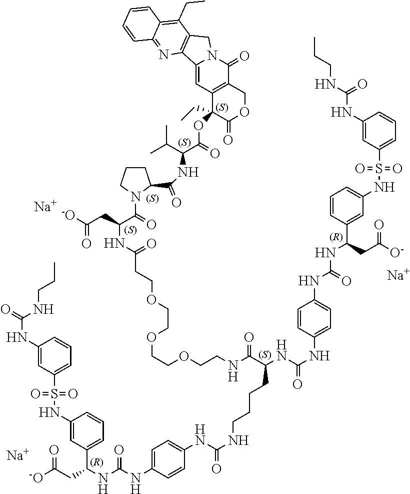

The starting material ((4S)-4,11-diethyl-3,14-dioxo-3,4,12,14-tetrahydro-1H-pyrano [3′,4′:6,7] indolizino[1,2-b]quinolin-4-yl N-[3-(2-{2-[2-(L-lysylamino) ethoxy]ethoxy} ethoxy) propanoyl]-L-alpha-aspartyl-L-prolyl-L-valinate trifluoroacetate (1:1)) (Intermediate 11) was dissolved in DMF (50 mL). ((3R)-3-{[(4-{[(4-nitrophenoxy)carbonyl]amino}phenyl)carbamoyl]amino}-3-{3-[({3-[(propylcarbamoyl)amino] phenyl}sulfonyl)amino]phenyl}propanoic acid) (Building Block 2) and DIEA (570.3 mg, 4412.3 μmol) were added. The reaction was stirred for 15 min at rt. The reaction was evaporated to dryness and the residue was separated by prep. HPLC. The substance precipitated in the sample loop, which was rinsed with DMF. The compound was repurified by dissolving in DMSO and applying to the prep. HPLC again. Clean fractions were combined and freeze-dried to give a beige foam, which was dissolved in dioxane /water (1:1, 50 mL). A sodium hydroxide solution (278 μl, 1.0 M, 278 μmol) was added. The solution was freeze-dried to give Compound A1 (220 mg, 92% purity, 41%) as a beige foam. LC-MS: Rₜ=3.20 min; MS (ESIpos): m/z=1090 [M+2H]²⁺. ¹H-NMR (600 MHz, DMSO-d₆): δ [ppm]=12.02 (s, 1H), 10.00-9.92 (m, 1H), 8.76-8.69 (m, 1H), 8.61-8.52 (m, 1H), 8.34-8.29 (m, 1H), 8.28 (d, 1H), 8.26-8.23 (m, 1H), 8.13-8.06 (m, 1H), 8.06-8.03 (m, 1H), 8.03-7.96 (m, 1H), 7.86 (t, 1H), 7.76-7.66 (m, 1H), 7.65-7.55 (m, 1H), 7.35-7.31 (m, 1H), 7.31-7.26 (m, 1H), 7.26-7.22 (m, 1H), 7.22-7.20 (m, 1H), 7.20-7.17 (m, 1H), 7.17-7.15 (m, 1H), 7.08 (t, 1H), 6.95 (br d, 1H), 6.73-6.67 (m, 1H), 6.49-6.35 (m, 1H), 6.24-6.06 (m, 1H), 5.48 (s, 1H), 5.37-5.29 (m, 1H), 4.99-4.94 (m, 1H), 4.94-4.88 (m, 1H), 4.84 (t, 1H), 4.19-4.13 (m, 1H), 4.04-3.97 (m, 1H), 3.84-3.74 (m, 1H), 3.57 (s, 1H), 3.61-3.53 (m, 1H), 3.50-3.42 (m, 1H), 3.39 (br d, 1H), 3.29-3.11 (m, 1H), 3.09-2.98 (m, 1H), 2.98-2.91 (m, 1H), 2.74-2.68 (m, 1H), 2.65-2.57 (m, 1H), 2.43-2.28 (m, 1H), 2.26-2.16 (m, 1H), 2.16-2.07 (m, 1H), 2.05-1.99 (m, 1H), 1.96-1.87 (m, 1H), 1.65-1.55 (m, 1H), 1.55-1.46 (m, 1H), 1.43-1.34 (m, 1H), 1.32-1.27 (m, 1H), 1.27-1.22 (m, 1H), 0.95-0.89 (m, 1H), 0.88 (br s, 1H), 0.83 (t, 1H).

### Example A2: Preparation of Trinatrium-N²,N⁶-bis(N-{[4-({[(1R)-2-carboxylato-1-{3-[({3-[(propylcarbamoyl)amino]phenyl} sulfonyl)amino]phenyl}ethyl]carbamoyl}amino) phenyl]carbamoyl}-beta-alanyl)-N-{(14S)-14-(carboxylatomethyl)-15-[(2S)-2-{[(2S)-1-{[(4S)-4,11-diethyl-3,14-dioxo-3,4,12,14-tetrahydro-1H-pyrano[3′,4′:6,7]indolizino[1,2-b]chinolin-4-yl]oxy}-3-methyl-1-oxobutan-2-yl]carbamoyl} pyrrolidin-1-yl]-12,15-dioxo-3,6,9-trioxa-13-azapentadec-1-yl}-L-lysinamid (Compound A2)

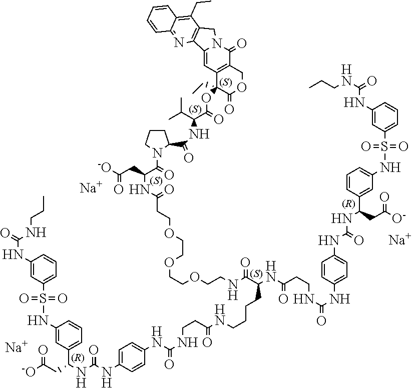

(4S)-4,11-diethyl-3,14-dioxo-3,4,12,14-tetrahydro-1H-pyrano[3′,4′:6,7]indolizino[1,2-b]quinolin-4-yl N-{3-[2-(2-{2-[(N2,N6-di-beta-alanyl-L-lysyl)amino]ethoxy}ethoxy)ethoxy] propanoyl}-L-alpha-aspartyl-L-prolyl-L-valinate (Intermediate 13) (174.0 mg, 149.8 μmol) was dissolved in DMF (34 mL). (3R)-3-{[(4-{[(4-nitrophenoxy)carbonyl]amino}phenyl)carbamoyl] amino}-3-{3-[({3-[(propylcarbamoyl) amino]phenyl}sulfonyl) amino]phenyl}propanoic acid (Building Block 2) (237.2 mg, 329.6 μmol) and DIEA (521.9 μL, 2996.6 μmol) were added. The reaction was stirred for 30 min at rt. The reaction was evaporated to dryness and the residue was dissolved in DMSO and purified by prep. HPLC. Due to low solubilities the HPLC flow had to be reduced, clean fractions were combined and freeze-dried to give a beige foam, which was dissolved in dioxane/water (1:1, 50 mL). A sodium hydroxide solution (304 μl, 1.0 M, 304 μmol) was added. The solution was freeze-dried to give Compound A2 (230 mg, 93% purity, 59%) as a beige foam. LC-MS: Rt=3.11 min; MS (ESIpos): m/z=1160 [M+2H]²⁺. ¹H-NMR (600 MHz, DMSO-d₆): δ [ppm]=11.31-11.15 (m, 1H), 9.98-9.87 (m, 1H), 8.88-8.80 (m, 1H), 8.80-8.71 (m, 1H), 8.68-8.56 (m, 1H), 8.28 (s, 1H), 8.23-8.12 (m, 2H), 8.03 (d, 1H), 8.01-7.94 (m, 1H), 7.92-7.88 (m, 1H), 7.88-7.82 (m, 1H), 7.71 (t, 1H), 7.51 (br s, 1H), 7.36-7.31 (m, 1H), 7.31-7.27 (m, 1H), 7.26-7.22 (m, 2H), 7.22-7.19 (m, 2H), 7.17 (d, 1H), 7.06 (t, 1H), 6.94 (br d, 1H), 6.64 (br d, 1H), 5.46 (s, 1H), 5.33 (br d, 1H), 5.13-5.07 (m, 1H), 5.00-4.93 (m, 1H), 4.93-4.83 (m, 1H), 4.22-4.15 (m, 1H), 4.00-3.93 (m, 1H), 3.90-3.81 (m, 1H), 3.65-3.58 (m, 1H), 3.57 (s, 2H), 3.56-3.51 (m, 1H), 3.50-3.40 (m, 5H), 3.40-3.36 (m, 2H), 3.27-3.21 (m, 3H), 3.21-3.13 (m, 2H), 3.06-2.96 (m, 1H), 2.96-2.89 (m, 2H), 2.74-2.65 (m, 1H), 2.63-2.56 (m, 1H), 2.57-2.52 (m, 12H), 2.43-2.37 (m, 1H), 2.36-2.29 (m, 1H), 2.29-2.19 (m, 3H), 2.15-2.06 (m, 1H), 2.04-1.98 (m, 1H), 1.98-1.91 (m, 1H), 1.91-1.83 (m, 1H), 1.65-1.55 (m, 1H), 1.53-1.43 (m, 1H), 1.40-1.33 (m, 3H), 1.33-1.28 (m, 2H), 1.28-1.20 (m, 1H), 0.93-0.90 (m, 2H), 0.89 (br d, 2H), 0.83 (t, 3H)

### Example A3: Preparation of Trisodium (4S)-4,11-diethyl-3,14-dioxo-3,4,12,14-tetrahydro-1H-pyrano[3′,4′:6,7]indolizino [1,2-b]quinolin-4-yl 1-[(2S)-15-{[N²,N⁶-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-L-valinate (Compound A3)

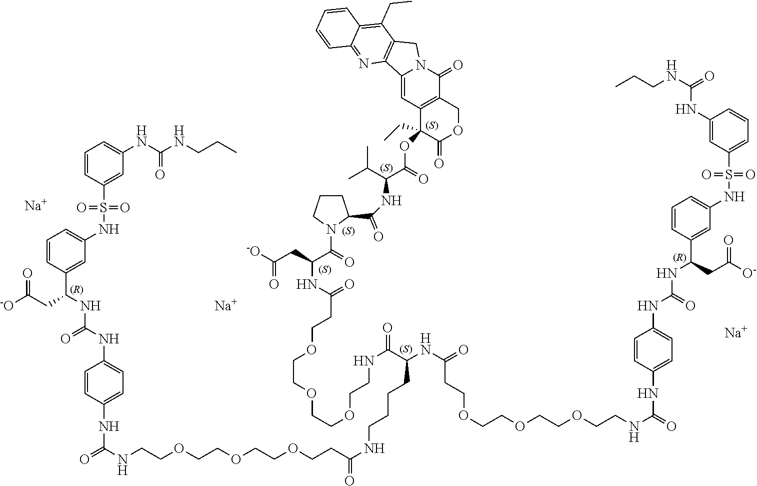

(4S)-4,11-Diethyl-3,14-dioxo-3,4,12,14-tetrahydro-1H-pyrano[3′,4′:6,7]indolizino [1,2-b]quinolin-4-yl N-(3-{2-[2-(2-{[N2,N6-bis(3-{2-[2-(2-aminoethoxy) ethoxy]ethoxy}propanoyl)-L-lysyl]amino} ethoxy) ethoxy]ethoxy}propanoyl) -L-alpha-aspartyl-L-prolyl-L-valinate trifluoroacetate (1:2) (Intermediate 12) (30 mg, 18.1 μmol) was dissolved in DMF (4 mL). (3R)-3-{[(4-{[(4-nitrophenoxy) carbonyl]amino} phenyl)carbamoyl] amino}-3-{3-[({3-[(propylcarbamoyl) amino]phenyl} sulfonyl) amino]phenyl}propanoic acid (Building Block 2) (28.8 mg, 39.9 μmol) and DIEA (63.2 μL, 362.8 μmol) were added. The reaction was stirred for 30 min at rt. The reaction was evaporated to dryness. The residue was dissolved in DMSO and purified by prep. HPLC to yield a yellow foam, which was dissolved in dioxane/water (1:1, 4.0 mL). A sodium hydroxide solution (93 μl, 0.10 M, 9.3 μmol) was added. The solution was freeze-dried to give Compound A3 (6.21 mg, 100% purity, 13%) as a colorless foam. LC-MS: Rₜ=4.16 min; MS (ESIpos): m/z=1293 [M+2H]²+. 1H-NMR (600 MHz, DMSO-d₆): δ [ppm]=11.17-11.04 (m, 1H), 9.98-9.81 (m, 1H), 8.83 (br s, 1H), 8.67-8.49 (m, 2H), 8.27 (br d, 1H), 8.25-8.19 (m, 1H), 8.15 (br d, 1H), 8.03 (br t, 1H), 7.95 (br s, 1H), 7.89-7.78 (m, 2H), 7.72 (br t, 1H), 7.54 (br s, 1H), 7.47 (br s, 1H), 7.35-7.16 (m, 7H), 7.12-7.01 (m, 1H), 7.00-6.88 (m, 1H), 6.66 (br d, 1H), 6.38-6.26 (m, 1H), 5.47 (br s, 1H), 5.38-5.27 (m, 1H), 5.09-5.01 (m, 1H), 4.97 (br s, 1H), 4.88 (br d, 1H), 4.19 (br d, 1H), 4.02-3.88 (m, 1H), 3.88-3.75 (m, 1H), 3.62-3.54 (m, 5H), 3.53-3.40 (m, 18H), 3.27-3.16 (m, 6H), 3.12-3.01 (m, 1H), 3.00-2.91 (m, 3H), 2.70 (br dd, 1H), 2.60 (br d, 1H), 2.56-2.52 (m, 3H), 2.46-2.34 (m, 3H), 2.33-2.26 (m, 3H), 2.26-2.07 (m, 2H), 2.05-1.96 (m, 1H), 1.96-1.80 (m, 1H), 1.62-1.53 (m, 1H), 1.49-1.09 (m, 7H), 0.91 (br d, 4H), 0.83 (br t, 3H)

### Example A4: Preparation of Trisodium (4S)-4,11-diethyl-3,14-dioxo-3,4,12,14-tetrahydro-1H-pyrano[3′,4′:6,7]indolizino [1,2-b]quinolin-4-yl 1-[(2S)-15-{[N²,N⁶-bis(14-{4-[({(1S)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-L-valinate (Compound A4)

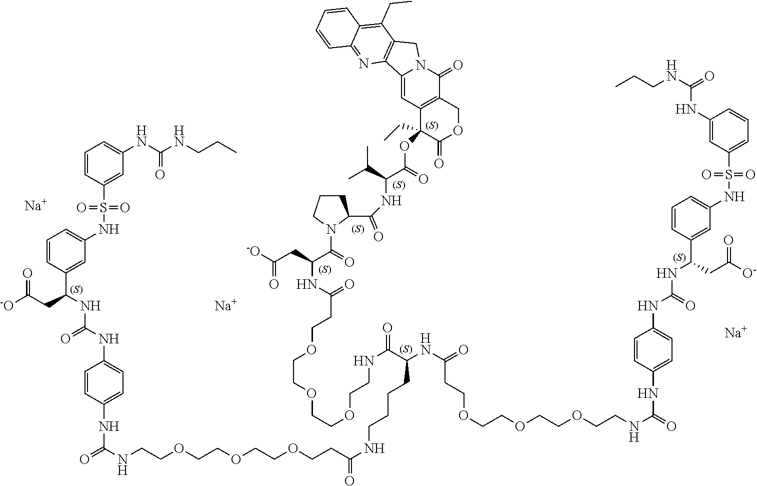

Compound A4 is the epimer of Compound A3 and was synthesized in analogy to Example A3 employing the enantiomeric Building block 11 instead of building block 2. Yield: 3.3 mg (100% purity, 7% yield over 2 steps). LC-MS: Rₜ=4.16 min; MS (ESIpos): m/z=1293 [M+2H]²⁺.

### Example A5: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(14-{4-[({(1R)-2-carboxy-1-[3-({3-[(propylcarbamoyl) amino]benzene-1-sulfonyl}amino)phenyl]ethyl}carbamoyl)amino]anilino}-14-oxo-4,7,10-trioxa-13-azatetradecanan-1-oyl)-L-lysyl]amino}ethyl)(methyl)amino] ethyl}(methyl)amino]ethyl}-N-methylglycyl-L-asparaginyl-L-prolyl-L-valinate (Compound A5)

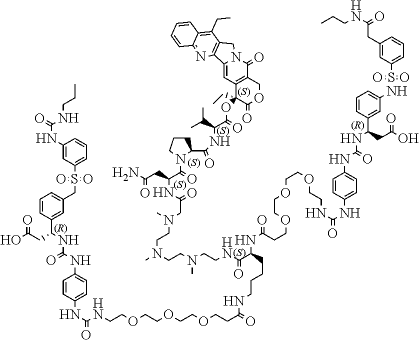

Trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[(2-{[N²,N⁶-bis(3-{2-[2-(2-aminoethoxy)ethoxy] ethoxy}propanoyl) -L-lysyl]amino}ethyl)(methyl)amino]ethyl} (methyl) amino]ethyl}-N-methylglycyl-L-asparaginyl-L-prolyl-L-valinate (2/1) (Intermediate 20) (28.0 mg, 16.7 μmol) was dissolved in DMF (5.0 mL). (3R)-3-{[(4-{[(4-nitrophenoxy)carbonyl]amino}phenyl)carbamoyl]amino}-3-[3-({3-[(propylcarbamoyl) amino]benzene-1-sulfonyl}amino)phenyl]propanoic acid (Building Block 2) (24.0 mg, 33.4 μmol) and DIEA (58 μl, 330 μmol) were added. The reaction was stirred for 30 min at RT. The reaction was concentrated in vacuo. The residue was separated by prep. HPLC to give Compound A5 (21.0 mg, 100% purity, 48% yield) as a yellow foam. LC-MS: Rₜ=3.32 min; MS (ESIpos): m/z=1306 [M+2H]²⁺. ¹H NMR (600 MHz, DMSO-d₆) δ ppm 0.835-0.877 (m, 3H) 0.889-0.931 (m, 1H) 0.955 (d, J=6.65 Hz, 2H) 1.046 (t, J=6.85 Hz, 1H) 1.164-1.255 (m, 1H) 1.308 (br t, J=7.63 Hz, 1H) 1.333-1.381 (m, 1H) 1.397-1.458 (m, 2H) 1.465-1.559 (m, 1H) 1.568-1.678 (m, 1H) 1.917-2.044 (m, 1H) 2.119-2.256 (m, 2H) 2.269-2.315 (m, 2H) 2.315-2.390 (m, 1H) 2.407-2.479 (m, 1H) 2.542 (s, 5H) 2.610-2.698 (m, 3H) 2.719-2.803 (m, 3H) 2.817 (s, 2H) 2.974-3.066 (m, 3H) 3.135-3.278 (m, 6H) 3.309-3.408 (m, 3H) 3.408-3.444 (m, 5H) 3.444-3.470 (m, 4H) 3.503-3.526 (m, 8H) 3.551 (br s, 5H) 3.557-3.596 (m, 5H) 3.596-3.698 (m, 4H) 3.723-3.789 (m, 1H) 3.845-3.903 (m, 1H) 4.063-4.138 (m, 1H) 4.826-4.878 (m, 1H) 4.964-5.022 (m, 1H) 5.303-5.395 (m, 1H) 5.495 (s, 1H) 6.070-6.100 (m, 1H) 6.217-6.267 (m, 1H) 6.653 (br d, J=8.22 Hz, 1H) 6.898 (br d, J=8.80 Hz, 1H) 6.986 (br d, J=7.82 Hz, 1H) 7.016 (br s, 1H) 7.141 (br s, 1H) 7.147 (br s, 1H) 7.154 (br s, 1H) 7.158-7.186 (m, 1H) 7.186-7.229 (m, 3H) 7.238 (br s, 1H) 7.243-7.262 (m, 1H) 7.262-7.293 (m, 1H) 7.337-7.368 (m, 1H) 7.402 (br d, J=8.41 Hz, 1H) 7.537 (br d, J=0.78 Hz, 1H) 7.716-7.756 (m, 1H) 7.803-7.834 (m, 1H) 7.834-7.872 (m, 1H) 8.026 (d, J=8.41 Hz, 1H) 8.054-8.089 (m, 1H) 8.158-8.224 (m, 1H) 8.288 (t, J=7.69 Hz, 1H) 8.352 (s, 1H) 8.372 (s, 1H) 8.568 (s, 1H) 8.773 (s, 1H) 8.790 (s, 1H) 8.936-9.068 (m, 1H) 10.268 (s, 1H) 10.523-10.649 (m, 1H) 11.910-12.817 (m, 1H).

### Example A6: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(14-{4-[({(1S)-2-carboxy-1-[3-({3-[(propylcarbamoyl) amino] benzene-1-sulfonyl}amino)phenyl]ethyl}carbamoyl)amino]anilino}-14-oxo-4,7,10-trioxa-13-azatetradecanan-1-oyl)-L-lysyl]amino}ethyl) (methyl)amino]ethyl}(methyl) amino]ethyl}-N-methylglycyl-L-asparaginyl-L-prolyl-L-valinate (Compound A6)

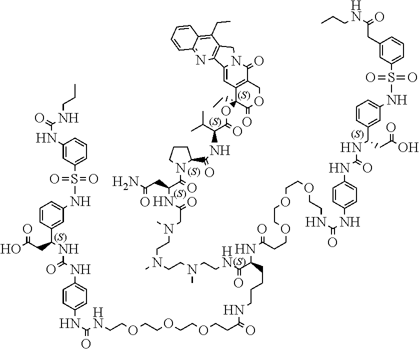

Trifluoroacetic acid-(4S)-4,11-diethyl-3,14-dioxo-3,4,12,14-tetrahydro-1H-pyrano[3′,4′:6,7] indolizino [1,2-b]quinolin-4-yl N-{2-[{2-[(2-{[N²,N⁶-bis(3-{2-[2-(2-aminoethoxy)ethoxy] ethoxy}propanoyl)-L-lysyl]amino} ethyl)(methyl)amino]ethyl}(methyl)amino]ethyl}-N-methylglycyl-L-asparaginyl-L-prolyl-L-valinate (2/1) (Intermediate 20) (28.0 mg, 16.7 μmol) was dissolved in DMF (5.0 mL). (3S)-3-{[(4-{[(4-nitrophenoxy)carbonyl]amino}phenyl)carbamoyl]amino}-3-[3-({3-[(propylcarbamoyl) amino]benzene-1-sulfonyl}amino)phenyl]propanoic acid (Building Block 11) (26.4 mg, 36.7 μmol) and DIEA (58 μl, 330 μmol; CAS-RN:[7087-68-5]) were added. The reaction was stirred for 30 min at RT and then concentrated in vacuo. The residue was separated by prep. HPLC to give Compound A6 (18.0 mg, 100% purity, 41% yield) as a yellow foam. LC-MS: Rₜ=3.33 min; MS (ESIpos): m/z=1306 [M+2H]⁺. ¹H NMR (600 MHz, DMSO-d₆) δ ppm 0.855 (t, J=7.43 Hz, 3H) 0.879-0.939 (m, 2H) 0.955 (d, J=6.85 Hz, 3H) 1.046 (br t, J=7.04 Hz, 1H) 1.198-1.287 (m, 1H) 1.308 (t, J=7.63 Hz, 2H) 1.330-1.379 (m, 1H) 1.397-1.458 (m, 2H) 1.464-1.544 (m, 1H) 1.566-1.665 (m, 1H) 1.900-2.067 (m, 2H) 2.148-2.243 (m, 2H) 2.269-2.390 (m, 3H) 2.406-2.467 (m, 1H) 2.542 (s, 7H) 2.636 (br d, J=7.04 Hz, 2H) 2.717-2.838 (m, 5H) 2.971-3.050 (m, 4H) 3.135-3.296 (m, 7H) 3.401-3.446 (m, 6H) 3.446-3.499 (m, 14H) 3.554-3.611 (m, 8H) 3.722-3.789 (m, 2H) 3.847-3.899 (m, 1H) 4.024-4.176 (m, 1H) 4.829-4.865 (m, 1H) 4.954-5.032 (m, 2H) 5.306-5.390 (m, 1H) 5.495 (s, 1H) 6.049-6.115 (m, 1H) 6.248 (t, J=6.11 Hz, 1H) 6.653 (br d, J=8.41 Hz, 1H) 6.897 (m, J=8.22 Hz, 1H) 6.986 (d, J=8.02 Hz, 1H) 7.003-7.032 (m, 1H) 7.141 (br s, 1H) 7.148 (br s, 1H) 7.151-7.162 (m, 1H) 7.162-7.174 (m, 1H) 7.207 (s, 3H) 7.231-7.243 (m, 1H) 7.249 (d, J=1.56 Hz, 1H) 7.265 (s, 1H) 7.278 (s, 1H) 7.291 (s, 1H) 7.402 (m, J=8.22 Hz, 1H) 7.536 (br d, J=0.98 Hz, 1H) 7.717-7.759 (m, 1H) 7.819 (br t, J=5.48 Hz, 1H) 7.834-7.873 (m, 1H) 8.025 (d, J=8.41 Hz, 1H) 8.065 (br t, J=1.86 Hz, 1H) 8.167-8.226 (m, 1H) 8.290 (t, J=7.40 Hz, 1H) 8.352 (s, 1H) 8.373 (s, 1H) 8.790 (s, 1H) 8.949-9.065 (m, 1H) 10.267 (s, 1H) 12.087-12.532 (m, 1H).

### Example A7: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(N-{2-[{2-[{2-[({4-[({(1R)-2-carboxy-1-[3-({3-[(propylcarbamoyl) amino]benzene-1-sulfonyl}amino) phenyl]ethyl}carbamoyl)amino]phenyl}carbamoyl)amino]ethyl}(methyl)amino]ethyl}I(methyl)amino]ethyl}-N-methylglycyl)-L-lysyl]amino}ethyl)(methyl)amino]ethyl}((methyl)amino]ethyl}-N-methylglycyl-L-asparaginyl-L-prolyl-L-valinate (Compound A7)

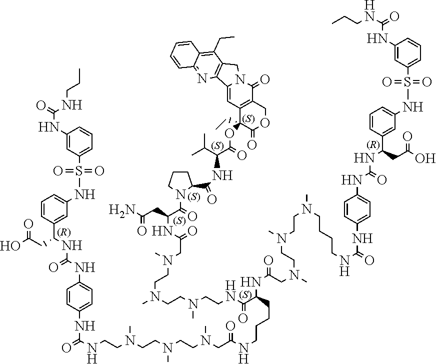

Trifluoroacetic acid-(4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(N-{2-[{2-[(2-aminoethyl)(methyl)amino]ethyl} (methyl)amino]ethyl}-N-methylglycyl)-L-lysyl]amino} ethyl) 5 (methyl)amino]ethyl} (methyl)amino]ethyl}-N-methylglycyl-L-asparaginyl-L-prolyl-L-valinate (2/1) (Intermediate 22) (8.00 mg, 4.63 μmol) was dissolved in DMF (2.0 mL). (3R)-3-{[(4-{[(4-Nitrophenoxy)carbonyl] amino}phenyl)carbamoyl]amino}-3-[3-({3-[(propylcarbamoyl)amino]benzene-1-sulfonyl} amino)phenyl]propanoic acid (Building Block 2) (6.66 mg, 9.26 μmol) and DIEA (16 μl, 93 μmol) were added. The reaction was stirred for 30 min at RT and then concentrated in vacuo. The residue was separated twice by prep. HPLC to give Compound A7 (5.50 mg, 10000 purity, 45% yield) as a yellow foam. LC-MS: Rₜ=1.79 min; MS (ESIpos): m/z=1331 [M+2H]². ¹H NMR (600 MHz, DMSO-d₆) δ ppm 0.855 (t, J=7.43 Hz, 4H) 0.878-0.936 (m, 2H) 0.953 (d, J=6.85 Hz, 3H) 1.310 (br t, J=7.73 Hz, 2H) 1.386-1.459 (m, 3H) 1.484-1.600 (m, 1H) 1.628-1.756 (m, 1H) 1.912-2.028 (in, 2H) 2.147-2.273 (m, 3H) 2.273-2.356 (m, 5H) 2.542 (s, 8H) 2.635 (br d, J=7.43 Hz, 2H) 2.684-2.790 (m, 7H) 2.790-2.877 (m, 10H) 3.002-3.053 (m, 3H) 3.053-3.097 (m, 2H) 3.152-3.253 (m, 8H) 3.269-3.326 (m, 4H) 3.472-3.660 (m, 26H) 3.736-3.799 (m, 4H) 4.096 (br t, J=8.02 Hz, 1H) 4.207-4.256 (m, 1H) 4.850 (dd, J=8.41, 3.33 Hz, 1H) 4.965-5.018 (m, 2H) 5.305-5.396 (m, 1H) 5.496 (s, 1H) 6.339-6.370 (m, 1H) 6.649-6.688 (m, 1H) 6.761 (br dd, J=9.78, 0.98 Hz, 1H) 6.884 (dd, J=8.12, 1.08 Hz, 1H) 6.985 (d, J=7.63 Hz, 1H) 7.005 (br s, 1H) 7.134-7.157 (m, 1H) 7.166 (d, J=2.35 Hz, 1H) 7.228-7.237 (m, 1H) 7.242 (s, 4H) 7.253 (s, 1H) 7.262 (s, 1H) 7.275 (s, 1H) 7.288 (s, 1H) 7.404 (br d, J=7.63 Hz, 1H) 7.544 (br d, J=0.98 Hz, 1H) 7.737 (t, J=8.12 Hz, 1H) 7.830-7.874 (m, 1H) 8.029 (d, J=8.61 Hz, 1H) 8.085 (s, 1H) 8.289 (t, J=8.71 Hz, 1H) 8.482 (s, 1H) 8.528-8.595 (m, 1H) 8.772-8.811 (m, 1H) 8.882 (s, 1H) 8.923-9.029 (m, 1H) 10.264 (s, 1H).

### Example A8: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[(2-{[N²,N⁶-bis(N-{2-[{2-[{2-[({4-[({(1S)-2-carboxy-1-[3-({3-[(propylcarbamoyl) amino]benzene-1-sulfonyl}amino)phenyl]ethyl}carbamoyl)amino] phenyl}carbamoyl) amino]ethyl}(methyl)amino]ethyl}(methyl) amino]ethyl}-N-methylglycyl)-L-lysyl]amino}ethyl)(methyl)amino]ethyl}(methyl)amino]ethyl}-N-methylglycyl-L-asparaginyl-L-prolyl-L-valinate (Compound A8)

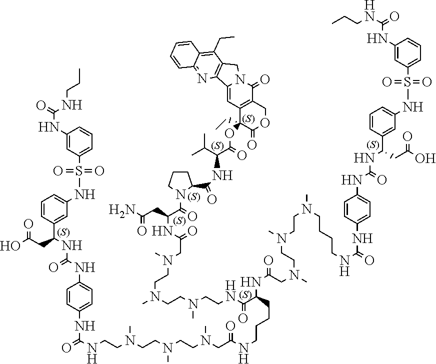

Trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[(2-{[N²,N⁶-bis(N-{2-[{2-[(2-aminoethyl)(methyl)amino] ethyl} (methyl)amino]ethyl}-N-methylglycyl)-L-lysyl]amino}ethyl) (methyl) amino]ethyl}(methyl) amino]ethyl}-N-methylglycyl-L-asparaginyl-L-prolyl-L-valinate (2/1) (Intermediate 22) (8.00 mg, 4.63 μmol) was dissolved in DMF (2.0 mL). (3S)-3-{[(4-{[(4-nitrophenoxy) carbonyl]amino} phenyl) carbamoyl] amino}-3-[3-({3-[(propylcarbamoyl)amino]benzene-1-sulfonyl}amino)phenyl]propanoic acid (Building block 11) (6.66 mg, 9.26 μmol) and DIEA (16 μl, 93 μmol; CAS-RN:[7087-68-5]) were added. The reaction was stirred for 30 min at RT and then concentrated in vacuo. The residue was separated twice by prep. HPLC to give Compound A8 (4.50 mg, 100% purity, 36% yield) as a yellow foam. LC-MS: Rₜ=1.74 min; MS (ESIpos): m/z=1331 [M+2H]²⁺. H NMR (600 MHz, DMSO-d₆) δ ppm 0.854 (t, J=7.43 Hz, 3H) 0.882-0.929 (m, 2H) 0.953 (d, J=6.65 Hz, 3H) 1.049 (br d, J=7.04 Hz, 1H) 1.178-1.258 (m, 1H) 1.309 (br t, J=7.63 Hz, 2H) 1.385-1.460 (m, 3H) 1.486-1.592 (m, 1H) 1.617-1.737 (m, 1H) 1.909-2.056 (m, 2H) 2.160-2.254 (m, 2H) 2.273-2.391 (m, 5H) 2.408-2.461 (m, 1H) 2.534-2.549 (m, 11H) 2.611-2.668 (m, 3H) 2.680-2.794 (m, 7H) 2.794-2.866 (m, 9H) 3.014-3.056 (m, 3H) 3.056-3.098 (m, 2H) 3.155-3.254 (m, 8H) 3.274-3.314 (m, 3H) 3.346-3.462 (m, 16H) 3.462-3.510 (m, 11H) 3.697-3.822 (m, 6H) 3.824-3.955 (m, 3H) 4.098 (br t, J=7.92 Hz, 1H) 4.201-4.272 (m, 1H) 4.829-4.869 (m, 1H) 4.959-5.023 (m, 2H) 5.351 (br d, J=8.22 Hz, 1H) 5.496 (s, 1H) 6.318-6.362 (m, 1H) 6.600-6.672 (m, 1H) 6.732-6.779 (m, 1H) 6.881 (d, J=8.17 Hz, 1H) 6.984 (d, J=7.63 Hz, 1H) 7.005 (br s, 1H) 7.132-7.158 (m, 1H) 7.166 (br d, J=3.91 Hz, 1H) 7.241 (s, 3H) 7.226-7.259 (m, 2H) 7.259-7.289 (m, 1H) 7.403 (br d, J=8.22 Hz, 1H) 7.523-7.563 (m, 1H) 7.716-7.759 (m, 1H) 7.831-7.871 (m, 1H) 8.029 (d, J=8.02 Hz, 1H) 8.083 (s, 1H) 8.285 (t, J=9.06 Hz, 1H) 8.475 (s, 1H) 8.529-8.594 (m, 1H) 8.772 (br s, 1H) 8.870 (s, 1H) 10.263 (s, 1H).

### Example A9: Preparation of (3R)-3-[({4-[({(20S,40S)-42-amino-20-[(17-{4-[({(1R)-2-carboxy-1-[3-({3-[(propylcarbamoyl)amino]benzene-1-sulfonyl} amino)phenyl]ethyl}carbamoyl)amino]anilino}-4,17-dioxo-7,10,13-trioxa-3,16-diazaheptadecan-1-yl)carbamoyl]-40-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-30,33,36-trimethyl-12,17,22,26,38,42-hexaoxo-3,6,9-trioxa-13,16,21,27,30,33,36,39-octaazadotetracontan-1-yl}carbamoyl)amino]phenyl}carbamoyl)amino]-3-[3-({3-[(propylcarbamoyl)amino]benzene-1-sulfonyl}amino)phenyl]propanoic acid (Compound A9)

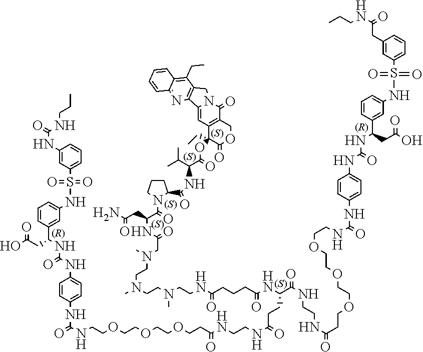

To a solution of trifluoroacetic acid (4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(2S,22S)-41-amino-2-(2-amino-2-oxoethyl)-22-[(15-amino-4-oxo-7,10,13-trioxa-3-azapentadecan-1-yl)carbamoyl]-6,9,12-trimethyl-4,16,20,25,30-pentaoxo-33,36,39-trioxa-3,6,9,12,15,21,26,29-octaazahentetracontanan-1-oyl}pyrrolidine-2-carbonyl]amino}-3-methylbutanoate (2/1) (7.00 mg, 3.73 μmol) (Intermediate 38) in DMF (4.0 ml) were added (3R)-3-{[(4-{[(4-nitrophenoxy)carbonyl]amino} phenyl)carbamoyl]amino}-3-[3-({3-[(propylcarbamoyl)amino]benzene-1-sulfonyl}amino) phenyl]propanoic acid (6.44 mg, 8.95 μmol) (Building Block 2) and N,N-diisopropylethylamine (5.2 μl, 30 μmol; CAS-RN:[7087-68-5]). The reaction was stirred for 2 hours at RT, concentrated in vacuo, and the residue was purified by prep. HPLC. Since the product was not clean it was repurified by prep. HPLC, then lyophilized to give Compound A9 (850 μg, 73% purity, 6% yield) as a yellow amorphous residue. LC-MS: Rₜ=3.23 min; MS (ESIpos): m/z=2808 [M+H]⁺.

### Example A10: Preparation of (3S)-3-[({4-[({(20S,40S)-42-amino-20-[(17-{4-[({(1S)-2-carboxy-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecan-1-yl)carbamoyl]-40-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-30,33,36-trimethyl-12,17,22,26,38,42-hexaoxo-3,6,9-trioxa-13,16,21,27,30,33,36,39-octaazadotetracontan-1-yl}carbamoyl)amino]phenyl}carbamoyl)amino]-3-[3-({3-[(propylcarbamoyl)amino]benzene-1-sulfonyl}amino)phenyl]propanoic acid (Compound A10)

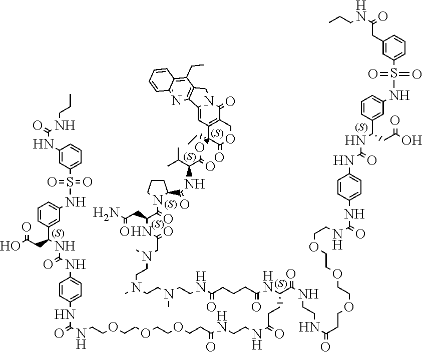

To a solution of trifluoroacetic acid (4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(2S,22S)-41-amino-2-(2-amino-2-oxoethyl)-22-[(15-amino-4-oxo-7,10,13-trioxa-3-azapentadecan-1-yl)carbamoyl]-6,9,12-trimethyl-4,16,20,25,30-pentaoxo-33,36,39-trioxa-3,6,9,12,15,21,26,29-octaazahentetracontanan-1-oyl}pyrrolidine-2-carbonyl]amino}-3-methylbutanoate (2/1) (7.00 mg, 90% purity, 3.36 μmol) (Intermediate 38) in DMF (4.0 ml) were added (3S)-3-{[(4-{[(4-nitrophenoxy)carbonyl] amino}phenyl)carbamoyl]amino}-3-[3-({3-[(propylcarbamoyl)amino]benzene-1-sulfonyl}amino)phenyl]propanoic acid (5.80 mg, 8.06 μmol) (Building Block 11) and N,N-diisopropylethylamine (4.7 μl, 27 μmol; CAS-RN:[7087-68-5]). The reaction was stirred for 2 hours at RT. The HPLC showed the desired product, but also many by-products. The reaction was concentrated in vacuo and the residue was purified by prep. HPLC twice and lyophilized to give Compound A10 (1.24 mg, 67% purity, 9% yield) as an amorphous residue. LC-MS: Rₜ=3.21 min; MS (ESIpos): m/z=1405 [M+2H]²⁺.

### Example A11: Preparation of (3R)-3-({[4-({[(18S)-39-{4-[({(1R)-2-carboxy-1-[3-({3-[(propylcarbamoyl)amino]benzene-1-sulfonyl}amino)phenyl]ethyl}carbamoyl)amino]anilino}-18-({20-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidin-1-yl]-9,12,15-trimethyl-5,17,20-trioxo-6,9,12,15,18-pentaazaicosanan-1-oyl}amino)-12,17,21,26,39-pentaoxo-3,6,9,29,32,35-hexaoxa-13,16,22,25,38-pentaazanonatriacontan-1-yl]carbamoyl}amino)phenyl]carbamoyl}amino)-3-[3-({3-[(propylcarbamoyl)amino]benzene-1-sulfonyl}amino)phenyl]propanoic acid (Compound

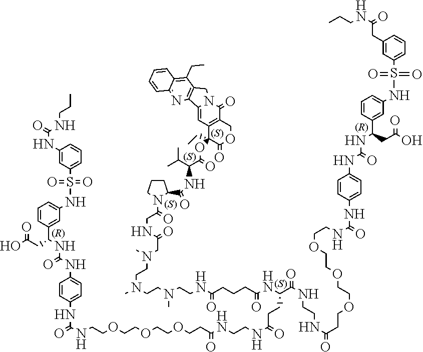

To a solution of trifluoroacetic acid (4S)-4,11-diethyl-3,14-dioxo-3,4,12,14-tetrahydro-1H-pyrano[3′,4′:6,7]indolizino[1,2-b]quinolin-4-yl (2S)-2-{[(2S)-1-{(22S)-41-amino-22-[(15-amino-4-oxo-7,10,13-trioxa-3-azapentadecan-1-yl)carbamoyl]-6,9,12-trimethyl-4,16,20,25,30-pentaoxo-33,36,39-trioxa-3,6,9,12,15,21,26,29-octaazahentetracontanan-1-oyl}pyrrolidine-2-carbonyl]amino}-3-methylbutanoate (2/1) (10.0 mg, 90% purity, 4.95 μmol) (Intermediate 32) in DMF (4.0 ml) was added Building Block 2 and N,N-diisopropylethylamine (6.9 μl, 40 μmol). The reaction was stirred for 2 hour at RT and concentrated in vacuo. The residue was purified bz prep. HPLC and lyophilized to give Compound A11 (5.30 mg, 100% purity, 39% yield) as a yellow amorphous residue.

LC-MS: Rₜ=3.27 min; MS (ESIpos): m/z=2751 [M+H]⁺

### Example A12: Preparation of (3S)-3-({[4-({[(18S)-39-{4-[({(1S)-2-carboxy-1-[3-({3-[(propylcarbamoyl)amino]benzene-1-sulfonyl}amino)phenyl]ethyl}carbamoyl)amino]anilino}-18-({20-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidin-1-yl]-9,12,15-trimethyl-5,17,20-trioxo-6,9,12,15,18-pentaazaicosanan-1-oyl}amino)-12,17,21,26,39-pentaoxo-3,6,9,29,32,35-hexaoxa-13,16,22,25,38-pentaazanonatriacontan-1-yl]carbamoyl}amino)phenyl]carbamoyl}amino)-3-[3-({3-[(propylcarbamoyl)amino]benzene-1-sulfonyl}amino)phenyl]propanoic acid (Compound A12)

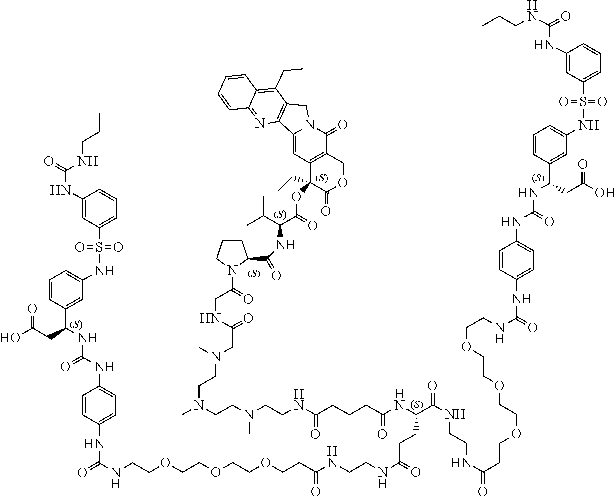

To a solution of Intermediate 32 (10.0 mg, 90% purity, 4.95 μmol) in DMF (4.0 ml) were added Building block 11 (8.54 mg, 11.9 μmol) and N,N-diisopropylethylamine (6.9 μl, 40 μmol; CAS-RN:[7087-68-5]). The reaction was stirred for 2 hours at RT and concentrated in vacuo. The residue was purified by prep. HPLC and lyophilized to give Compound A12 (3.60 mg, 91% purity, 24% yield) as a yellow amorphous residue. LC-MS: Rₜ=3.26 min; MS (ESIpos): m/z=2751 [M+H]⁺.

### Example A13: Preparation of trisodium (4S)-4,11-diethyl-3,14-dioxo-3,4,12,14-tetrahydro-1H-pyrano[3′,4′:6,7]indolizino[1,2-b]quinolin-4-yl 1-[(2S)-15-{[N2,N6-bis(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-lysyl]amino}-2-(carboxylatomethyl)-4-oxo-7,10,13-trioxa-3-azapentadecanan-1-oyl]-L-prolyl-L-alaninate (Compound A13)

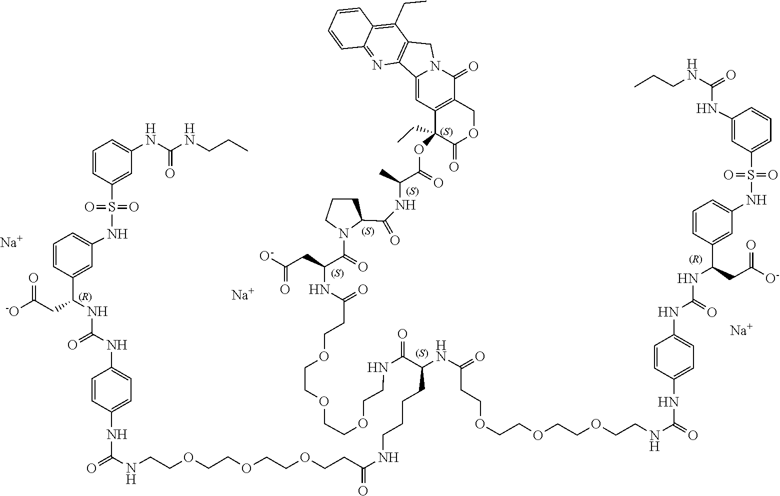

To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[2-(2-aminoethoxy)ethoxy]ethoxy}propanoyl)-L-alpha-aspartyl-L-prolyl-L-alaninate-trifluoroacetic acid (1/1) (Intermediate 67) in DMF was added Building Block 2 and N,N-diisopropylethylamine, as described herein. The resulting product was formulated as the sodium salt and lyophilized to afford Compound A13 according to the methods disclosed herein (see Example A3). LC-MS (Method 3): Rₜ=4.10 min; MS (ESIpos): m/z=2555 [M+H]⁺. ¹H-NMR (600 MHz, DMSO-d₆): 0.807 (4.07), 0.826 (9.22), 0.844 (4.77), 0.874 (1.46), 0.893 (2.92), 0.912 (1.55), 1.236 (1.16), 1.292 (1.62), 1.311 (3.34), 1.334 (2.94), 1.352 (4.25), 1.359 (3.49), 1.370 (4.14), 1.388 (2.05), 1.457 (0.61), 1.938 (1.10), 2.082 (0.43), 2.187 (0.75), 2.225 (1.08), 2.248 (0.98), 2.267 (1.57), 2.284 (2.67), 2.299 (1.42), 2.329 (0.78), 2.376 (2.01), 2.580 (1.36), 2.613 (0.61), 2.671 (0.79), 2.900 (1.23), 2.917 (2.53), 2.932 (2.58), 2.949 (1.32), 2.990 (1.06), 3.123 (0.78), 3.140 (0.93), 3.151 (1.21), 3.192 (3.58), 3.206 (3.64), 3.401 (4.82), 3.415 (5.47), 3.429 (3.70), 3.450 (6.85), 3.462 (11.97), 3.478 (7.31), 3.506 (16.00), 3.528 (1.82), 3.553 (2.57), 3.568 (12.93), 3.580 (3.17), 3.594 (2.27), 3.837 (0.71), 4.010 (0.40), 4.083 (0.42), 4.101 (0.66), 4.118 (0.45), 4.191 (0.56), 4.329 (0.44), 4.649 (0.41), 4.838 (0.87), 4.963 (1.00), 5.336 (1.50), 5.459 (2.30), 6.541 (0.55), 6.627 (1.67), 6.645 (1.61), 6.927 (1.47), 6.946 (1.90), 7.034 (1.57), 7.054 (2.41), 7.073 (1.06), 7.164 (1.70), 7.184 (2.33), 7.202 (1.72), 7.224 (5.04), 7.237 (5.77), 7.260 (1.58), 7.273 (1.91), 7.294 (2.53), 7.314 (1.28), 7.361 (0.99), 7.381 (1.02), 7.463 (1.75), 7.495 (2.88), 7.711 (0.73), 7.728 (0.49), 7.857 (1.29), 7.873 (3.07), 7.978 (0.71), 8.068 (1.27), 8.089 (1.26), 8.122 (0.64), 8.188 (1.60), 8.208 (1.46), 8.260 (0.83), 8.282 (0.78), 8.707 (0.87), 8.734 (0.88), 8.785 (1.26), 8.860 (2.02), 8.939 (0.47), 11.261 (2.05).

### Example B1: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)(methyl)amino]ethyl} (methyl)amino]ethyl}-N-methylglycyl-L-asparaginyl-L-prolyl-L-valinate (Compound B1)

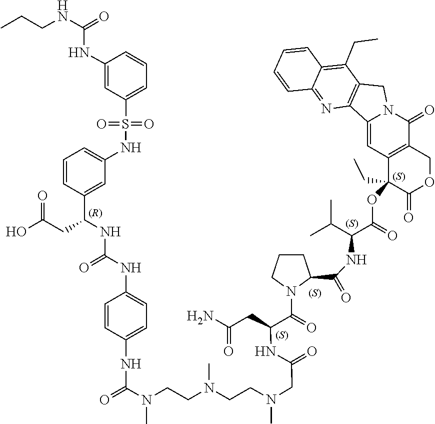

Trifluoroacetic acid (4S)-4,11-diethyl-3,14-dioxo-3,4,12,14-tetrahydro-1H-pyrano[3′,4′:6,7] indolizino[1,2-b]quinolin-4-yl N-methyl-N-(2-{methyl[2-(methylamino)ethyl]amino}ethyl)glycyl-L-asparaginyl-L-prolyl-L-valinate (Intermediate 3) (143 mg, 145 μmol) was dissolved in DMF (100 mL). (3R)-3-{[(4-{[(4-nitrophenoxy)carbonyl]amino}phenyl) carbamoyl]amino}-3-[3-({3-[(propylcarbamoyl) amino]benzene-1-sulfonyl}amino)phenyl]propanoic acid (Building Block 2) (104 mg, 145 μmol) and DIEA (510 μl, 2.9 mmol) were added. The reaction was stirred for 15 min at rt. The reaction was evaporated to dryness and the residue was separated by prep. HPLC to give Compound B1 (25 mg, 93% purity, 30%) as a light yellow foam. LC-MS: Rₜ=2.53 min; MS (ESIpos): m/z=1452 [M+H]⁺. ¹H-NMR (600 MHz, DMSO-d₆): δ [ppm]=12.46-12.14 (m, 1H), 10.26 (s, 1H), 8.79 (s, 1H), 8.43 (s, 1H), 8.29 (br d, 1H), 8.28 (br d, 1H), 8.25 (s, 1H), 8.07-8.05 (m, 1H), 8.02 (d, 1H), 7.85 (t, 1H), 7.73 (t, 1H), 7.50 (br s, 1H), 7.40 (br d, 1H), 7.30-7.29 (m, 1H), 7.32-7.28 (m, 1H), 7.28-7.25 (m, 3H), 7.25 (br d, 1H), 7.24-7.22 (m, 1H), 7.16 (s, 1H), 7.15-7.12 (m, 1H), 7.01 (br s, 1H), 7.00-6.97 (m, 1H), 6.89 (d, 1H), 6.70 (br d, 1H), 6.26 (br t, 1H), 5.49 (s, 2H), 5.40-5.29 (m, 2H), 5.00 (br d, 1H), 4.99-4.94 (m, 1H), 4.90-4.84 (m, 1H), 4.07 (br t, 1H), 3.80-3.71 (m, 5H), 3.28-3.20 (m, 5H), 3.06-3.01 (m, 3H), 2.99 (s, 3H), 2.95-2.88 (m, 1H), 2.84 (s, 3H), 2.71-2.58 (m, 4H), 2.54 (s, 11H), 2.48-2.32 (m, 3H), 2.26-2.13 (m, 4H), 2.03-1.97 (m, 1H), 1.97-1.89 (m, 2H), 1.47-1.39 (m, 2H), 1.31 (t, 3H), 1.04 (s, 1H), 0.95 (br d, 5H), 0.91 (br t, 3H), 0.85 (t, 3H).

### Example B2: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycylglycyl-L-prolyl-L-valinate (Compound B2)

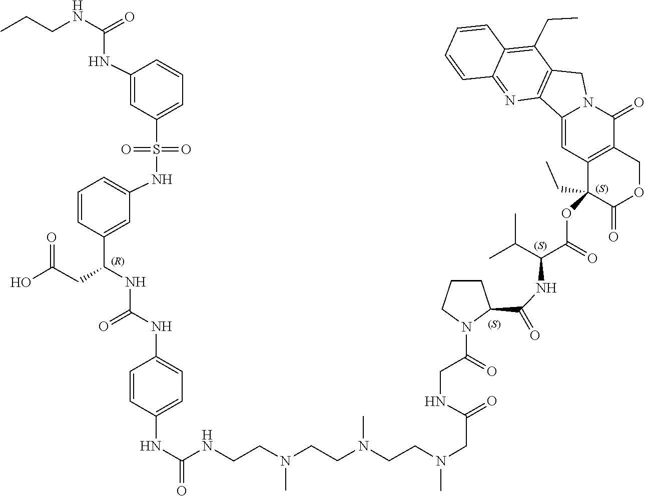

Trifluoroacetic acid (4S)-4,11-diethyl-3,14-dioxo-3,4,12,14-tetrahydro-1H-pyrano[3′,4′:6,7] indolizino[1,2-b]quinolin-4-yl N-{2-[{2-[(2-aminoethyl)(methyl)amino]ethyl}(methyl)amino] ethyl}-N-methylglycylglycyl-L-prolyl-L-valinate (Intermediate 4) (79.0 mg, 81.3 μmol) was dissolved in DMF (15 mL). (3R)-3-{[(4-{[(4-nitrophenoxy)carbonyl]amino} phenyl)carbamoyl]amino}-3-[3-({3-[(propylcarbamoyl)amino]benzene-1-sulfonyl}amino) phenyl]propanoic acid (Building Block 2) (64.3 mg, 89.4 μmol) and DIEA (280 μl, 1.6 mmol) were added. The reaction was stirred for 30 min at rt. The reaction was then evaporated to dryness and the residue was purified by prep. HPLC to give Compound B2 (91 mg, 93% purity, 72%) as a yellow foam. LC-MS: Rₜ=2.26 min; MS (ESIpos): m/z=1438 [M+H]⁺. ¹H-NMR (600 MHz, DMSO-d₆): δ [ppm]=12.45-12.16 (m, 1H), 11.10-11.02 (m, 1H), 10.26 (s, 1H), 8.81 (s, 1H), 8.77-8.70 (m, 1H), 8.70-8.63 (m, 1H), 8.45 (br d, 1H), 8.42 (s, 1H), 8.30 (d, 1H), 8.13-8.12 (m, 2H), 8.12-8.11 (m, 2H), 8.10 (d, 1H), 8.06 (s, 1H), 7.86 (t, 1H), 7.74 (t, 1H), 7.41 (br d, 1H), 7.29-7.26 (m, 1H), 7.26-7.24 (m, 1H), 7.24 (s, 3H), 7.23-7.21 (m, 1H), 7.16 (s, 1H), 7.15-7.13 (m, 1H), 7.07-7.04 (m, 1H), 6.98 (d, 1H), 6.95-6.92 (m, 3H), 6.89 (d, 1H), 6.69 (br d, 1H), 6.52-6.42 (m, 1H), 6.29-6.25 (m, 1H), 5.55-5.47 (m, 2H), 5.35 (br d, 2H), 5.02-4.97 (m, 1H), 4.58 (dd, 1H), 4.24 (dd, 1H), 4.12-4.07 (m, 1H), 4.05-3.95 (m, 2H), 3.95-3.87 (m, 2H), 3.66-3.51 (m, 6H), 3.28-3.14 (m, 9H), 3.06-3.01 (m, 3H), 2.90-2.79 (m, 7H), 2.79-2.71 (m, 3H), 2.68-2.58 (m, 3H), 2.54 (s, 14H), 2.52-2.52 (m, 1H), 2.45-2.36 (m, 1H), 2.34-2.26 (m, 2H), 2.26-2.22 (m, 1H), 2.22-2.15 (m, 3H), 2.05-1.94 (m, 2H), 1.91-1.84 (m, 1H), 1.46-1.39 (m, 2H), 1.31 (t, 3H), 1.00-0.97 (m, 4H), 0.97-0.94 (m, 3H), 0.94-0.89 (m, 3H), 0.85 (t, 3H).

### Example B3: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[{2-[({4-[({(1S)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino]ethyl}(methyl)amino]ethyl}-N-methylglycylglycyl-L-prolyl-L-valinate (Compound B3)

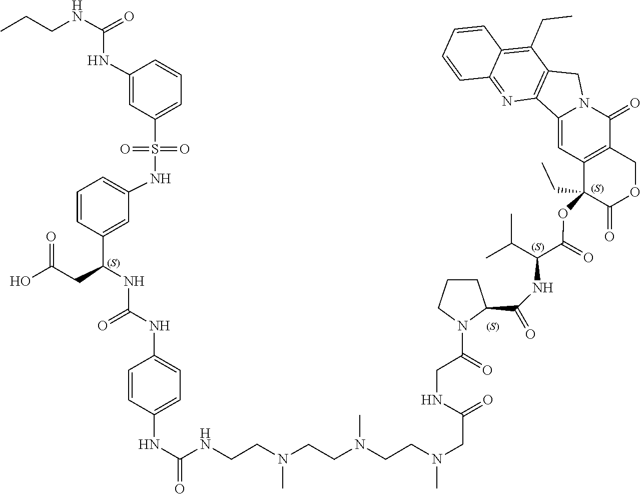

Compound B3 is the epimer of Compound B2 and was synthesized in analogy to Example B2, employing the enantiomeric Building block 11 instead of Building block 2. Yield: 17 mg (93% purity, 72% yield). LC-MS: Rₜ=2.28 min; MS (ESIpos): m/z=1438 [M+H]⁺. ¹H NMR (600 MHz, DMSO-d₆) δ ppm 0.833-1.012 (m, 13H) 1.213-1.335 (m, 1H) 1.309 (t, J=7.53 Hz, 3H) 1.356-1.457 (m, 3H) 1.842-1.910 (m, 1H) 1.932-2.063 (m, 2H) 2.147-2.254 (m, 4H) 2.272-2.426 (m, 4H) 2.542 (s, 12H) 2.604-2.677 (m, 3H) 2.714-2.911 (m, 10H) 3.008-3.052 (m, 3H) 3.092-3.262 (m, 9H) 3.521-3.703 (m, 6H) 3.808-3.938 (m, 2H) 3.956-4.028 (m, 1H) 4.043-4.113 (m, 1H) 4.192-4.274 (m, 1H) 4.574-4.608 (m, 1H) 4.976-5.015 (m, 1H) 5.349 (br d, J=8.02 Hz, 2H) 5.473-5.546 (m, 2H) 6.324 (br t, J=5.58 Hz, 1H) 6.499-6.582 (m, 1H) 6.714 (br d, J=8.02 Hz, 1H) 6.885 (d, J=8.22 Hz, 1H) 6.912-6.954 (m, 3H) 6.983 (br d, J=7.24 Hz, 1H) 7.043 (s, 1H) 7.052 (br d, J=2.35 Hz, 1H) 7.062 (s, 1H) 7.128-7.174 (m, 2H) 7.199-7.287 (m, 3H) 7.235 (s, 4H) 7.419 (br d, J=7.63 Hz, 1H) 7.739 (t, J=7.58 Hz, 1H) 7.851-7.883 (m, 1H) 8.057 (s, 1H) 8.087-8.135 (m, 3H) 8.297 (d, J=8.41 Hz, 1H) 8.423-8.480 (m, 2H) 8.710-8.786 (m, 2H) 8.888 (s, 1H) 10.264 (s, 1H) 11.044-11.109 (m, 1H) 12.206-12.352 (m, 1H).

### Example B4: Preparation of (4S)-4,11-Diethyl-3,14-dioxo-3,4,12,14-tetrahydro-1H-pyrano[3′,4′:6,7]indolizino[1,2-b]quinolin-4-yl N-{2-[{2-[{2-[({4-[({(1R)-2-carboxy-1-[3-({3-[(propylcarbamoyl)amino]benzene-1-sulfonyl}amino)phenyl]ethyl}carbamoyl)amino]phenyl}carbamoyl)amino]ethyl} (methyl)amino] ethyl} I(methyl)amino]ethyl}-N-methylglycyl-L-asparaginyl-L-prolyl-L-valinate (Compound B4)

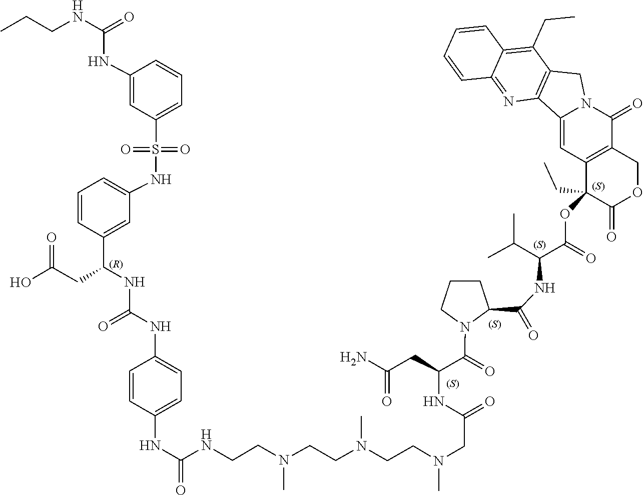

Trifluoroacetic acid (4S)-4,11-diethyl-3,14-dioxo-3,4,12,14-tetrahydro-1H-pyrano[3′,4′:6,7] indolizino[1,2-b]quinolin-4-yl N-{2-[{2-[(2-aminoethyl) (methyl)amino]ethyl}(methyl) amino]ethyl}-N-methylglycyl-L-asparaginyl-L-prolyl-L-valinate (Intermediate 4) (130 mg, 126 μmol) was initially dissolved in DMF (25 mL). (3R)-3-{[(4-{[(4-nitrophenoxy) carbonyl]amino}phenyl)carbamoyl] amino}-3-[3-({3-[(propylcarbamoyl)amino]benzene-1-sulfonyl}amino)phenyl]propanoic acid (Building Block 2) (100 mg, 139 μmol) and DIEA (440 μl, 2.5 mmol) were added. The reaction was stirred for 30 min at rt. The reaction was evaporated to dryness and the residue was separated by prep. HPLC to give Compound B4 (124 mg, 96% purity, 63%) as a yellow foam. LC-MS: Rₜ=2.94 min; MS (ESIpos): m/z=1495 [M+H]⁺. ¹H-NMR (600 MHz, DMSO-d₆): δ [ppm]=12.51-12.04 (m, 1H), 10.26 (s, 1H), 9.03-8.94 (m, 1H), 8.82 (s, 1H), 8.70 (s, 1H), 8.67-8.63 (m, 1H), 8.42 (br s, 1H), 8.29 (br dd, 2H), 8.15-8.10 (m, 1H), 8.06 (s, 1H), 8.02 (d, 1H), 7.85 (t, 1H), 7.74 (t, 1H), 7.54 (br s, 1H), 7.41 (d, 1H), 7.29-7.26 (m, 1H), 7.26-7.22 (m, 7H), 7.16 (s, 1H), 7.16-7.13 (m, 1H), 7.04-7.01 (m, 1H), 6.98 (br d, 1H), 6.94 (d, 1H), 6.89 (d, 1H), 6.71 (br d, 1H), 6.56-6.48 (m, 1H), 6.29 (br t, 1H), 5.49 (s, 2H), 5.39-5.30 (m, 2H), 4.99 (br t, 2H), 4.86-4.84 (m, 1H), 4.09 (t, 1H), 3.87-3.82 (m, 2H), 3.80-3.65 (m, 7H), 3.47-3.34 (m, 12H), 3.34-3.27 (m, 4H), 3.26-3.21 (m, 4H), 3.21-3.15 (m, 4H), 3.03 (q, 3H), 2.88-2.84 (m, 3H), 2.84-2.78 (m, 5H), 2.78-2.71 (m, 4H), 2.70-2.65 (m, 2H), 2.65-2.61 (m, 3H), 2.54 (s, 9H), 2.48-2.42 (m, 2H), 2.36-2.27 (m, 2H), 2.26-2.20 (m, 2H), 2.20-2.14 (m, 3H), 2.04-1.92 (m, 3H), 1.46-1.40 (m, 2H), 1.31 (t, 3H), 1.06-1.03 (m, 1H), 0.96 (br d, 6H), 0.93-0.89 (m, 4H), 0.85 (t, 4H).

### Example B4b: Preparation of (4S)-4,11-diethyl-3,14-dioxo-3,4,12,14-tetrahydro-1H-pyrano[3′,4′:6,7]indolizino[1,2-b]quinolin-4-yl N²-{13-[4-({[(1R)-2-carboxy-1-{3-[({3-[(propylcarbamoyl)amino]phenyl}sulfonyl)amino]phenyl}ethyl]carbamoyl}amino)anilino]-3,6,9-trimethyl-13-oxo-12-aza-3,6,9-triazoniatridecan-1-oyl}-L-asparaginyl-L-prolyl-L-valinate trichloride (Compound B4b)

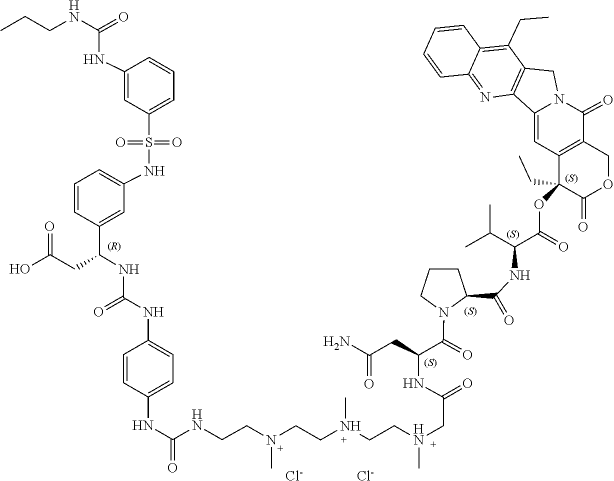

(4S)-4,11-Diethyl-3,14-dioxo-3,4,12,14-tetrahydro-1H-pyrano[3′,4′:6,7]indolizino [1,2-b]quinolin-4-yl N-{2-[(2-{[2-({[4-({[(1R)-2-carboxy-1-{3-[({3-[(propylcarbamoyl)amino]phenyl}sulfonyl)amino]phenyl}ethyl]carbamoyl}amino)phenyl]carbamoyl}amino)ethyl](methyl)amino}ethyl)(methyl)amino]ethyl}-N-methylglycyl-L-asparaginyl-L-prolyl-L-valinate (Compound B4) (130 mg, 126 μmol) was dissolved in dioxane/water (40 mL). A hydrochloric acid solution (277 μL, 1M) was added and the solution was freeze-dried to give Compound B4b (127 mg, 100% purity, 86%) as a light, yellow foam. LC-MS: Rₜ=2.92 min; MS (ESIpos): m/z=1495 [M+H]⁺. 1H-NMR (600 MHz, DMSO-d₆): δ [ppm]=10.27 (s, 1H), 9.00 (s, 2H), 8.78 (s, 1H), 8.49 (s, 1H), 8.29 (t, 2H), 8.04 (s, 1H), 8.03 (d, 2H), 7.86 (t, 1H), 7.73 (t, 1H), 7.57 (br s, 1H), 7.43 (br d, 1H), 7.28-7.21 (m, 7H), 7.17-7.13 (m, 2H), 7.04 (br s, 1H), 7.01-6.93 (m, 2H), 6.89 (d, 1H), 6.73 (br d, 1H), 5.49 (s, 2H), 5.34 (br d, 2H), 5.00 (br d, 2H), 4.07 (t, 2H), 3.77 (br t, 4H), 3.63-3.56 (m, 6H), 3.34-3.28 (m, 3H), 3.28-3.14 (m, 7H), 3.13-2.94 (m, 5H), 2.85 (s, 4H), 2.80 (br s, 4H), 2.72-2.59 (m, 4H), 2.55-2.52 (m, 9H), 2.49-2.35 (m, 3H), 2.25-2.13 (m, 5H), 1.96 (br d, 3H), 1.46-1.38 (m, 2H), 1.31 (t, 4H), 0.98-0.84 (m, 13H).

### Example B5: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[{2-[({4-[({(1S)-2-carboxy-1-[3-({3-[(propylcarbamoyl)amino]benzene-1-sulfonyl}amino)phenyl]ethyl}carbamoyl)amino]phenyl}carbamoyl)amino]ethyl}(methyl)amino] ethyl}(methyl)amino]ethyl}-N-methylglycyl-L-asparaginyl-L-prolyl-L-valinate (Compound B5)

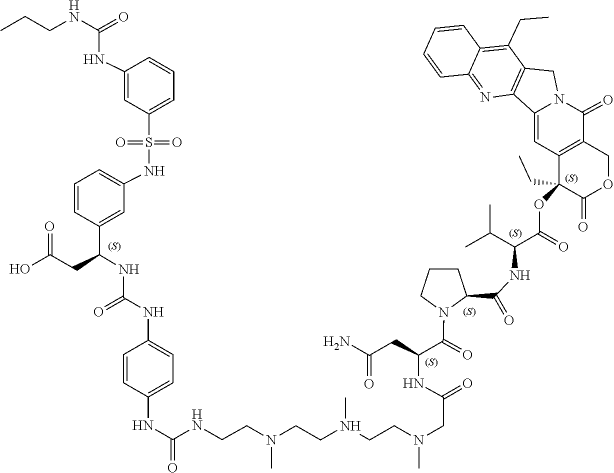

Compound B5 is the epimer of Compound B4 and was synthesized in analogy to Example B4, employing the enantiomeric Building block 11 instead of building block 2. Yield: 2.5 mg (100% purity, 17% yield). LC-MS: Rₜ=2.97 min; MS (ESIpos): m/z=1495 [M+H]⁺.

### Example B6: Preparation of disodium N-({4-[({(1R)-2-carboxylato-1-[3-({3-[(propylcarbamoyl)amino]benzene-1-sulfonyl}amino)phenyl]ethyl}carbamoyl)amino]phenyl}carbamoyl)-N-methylglycyl-N-methylglycyl-N-methylglycyl-N-{(2S)-3-carboxylato-1-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidin-1-yl]-1-oxopropan-2-yl}-N2-methylglycinamide (Compound B6)

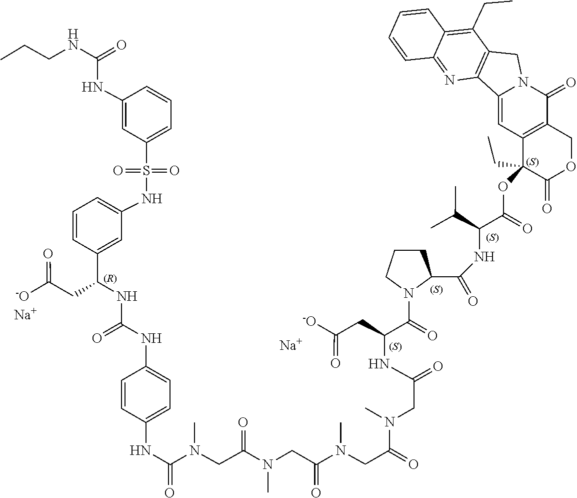

Step 1: To a solution of (4S)-4,11-diethyl-3,14-dioxo-3,4,12,14-tetrahydro-1H-pyrano[3′,4′:6,7]indolizino[1,2-b]quinolin-4-yl N-methylglycyl-N-methylglycyl-N-methylglycyl-N-methylglycyl-L-alpha-aspartyl-L-prolyl-L-valinate·trifluoroacetic acid (1/1) (55.0 mg, 97% purity, 49.1 μmol) (Intermediate 48) in DMF (10 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (40.9 mg, 95% purity, 54.0 μmol) (Building Block 2) and DIEA (43 μl, 250 μmol). The mixture was stirred at rt for 15 min and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-({4-[({(1R)-2-carboxy-1-[3-({3-[(propylcarbamoyl)amino]benzene-1-sulfonyl}amino)phenyl]ethyl}carbamoyl)amino]phenyl}carbamoyl)-N-methylglycyl-N-methylglycyl-N-methylglycyl-N-methylglycyl-L-alpha-aspartyl-L-prolyl-L-valinate (54.0 mg, 97% purity, 69% yield). LC-MS (Method 3): Rₜ=3.86 min; MS (ESIpos): m/z=1552 [M+H]⁺.
Step 2: To a solution of(4S)-4,11-diethyl-3,14-dioxo-3,4,12,14-tetrahydro-1H-pyrano[3′,4′:6,7]indolizino[1,2-b]quinolin-4-yl N-({4-[({(1R)-2-carboxy-1-[3-({3-[(propylcarbamoyl)amino]benzene-1-sulfonyl}amino)phenyl]ethyl}carbamoyl)amino]phenyl}carbamoyl)-N-methylglycyl-N-methylglycyl-N-methylglycyl-N-methylglycyl-L-alpha-aspartyl-L-prolyl-L-valinate (54.0 mg, 97% purity, 33.9 μmol) in dioxane/water (1:1, 8 mL) was added a sodium hydroxide solution (71 μl, 1 M, 71 μmol). The solution was freeze-dried to give Compound B6 (56.0 mg, 93% purity, 97%). LC-MS (Method 3): Rₜ=3.87 min; MS (ESIpos): m/z=1552 [M+H]⁺. ¹H-NMR (600 MHz, DMSO-d₆) δ[ppm]: 0.805 (3.56), 0.823 (7.64), 0.842 (4.15), 0.890 (4.08), 0.916 (5.40), 0.929 (6.26), 1.231 (0.59), 1.286 (2.44), 1.304 (4.87), 1.323 (2.77), 1.335 (1.91), 1.353 (2.90), 1.371 (2.90), 1.389 (1.51), 1.914 (1.19), 2.194 (1.51), 2.326 (1.45), 2.365 (0.86), 2.658 (1.78), 2.709 (1.58), 2.742 (2.50), 2.762 (1.71), 2.780 (3.42), 2.796 (2.17), 2.828 (2.90), 2.865 (3.69), 2.907 (6.19), 2.919 (6.12), 2.934 (6.06), 2.953 (2.57), 2.967 (1.84), 3.227 (3.36), 3.564 (16.00), 3.864 (0.92), 3.941 (2.11), 4.098 (0.99), 4.202 (2.04), 4.340 (0.99), 4.863 (0.59), 4.974 (1.45), 5.332 (2.63), 5.470 (4.08), 6.644 (1.25), 6.664 (1.32), 6.928 (1.12), 6.948 (1.45), 7.044 (1.32), 7.061 (1.91), 7.081 (0.92), 7.168 (1.51), 7.187 (2.04), 7.256 (7.84), 7.283 (2.70), 7.412 (0.72), 7.537 (1.05), 7.730 (1.12), 7.836 (1.51), 8.023 (1.05), 8.117 (1.45), 8.261 (1.38), 8.283 (1.32), 8.684 (0.53), 8.839 (0.66), 11.133 (0.66).

### Example C1: Preparation of (16S)-1-{4-[({(1R)-2-carboxy-1-[3-({3-[(propylcarbamoyl)amino]benzene-1-sulfonyl}amino) phenyl]ethyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N-trimethyl-1,14,18-trioxo-5,8,11,19-tetraoxa-2,15-diazahenicosan-21-aminium trifluoroacetate (Compound C1)

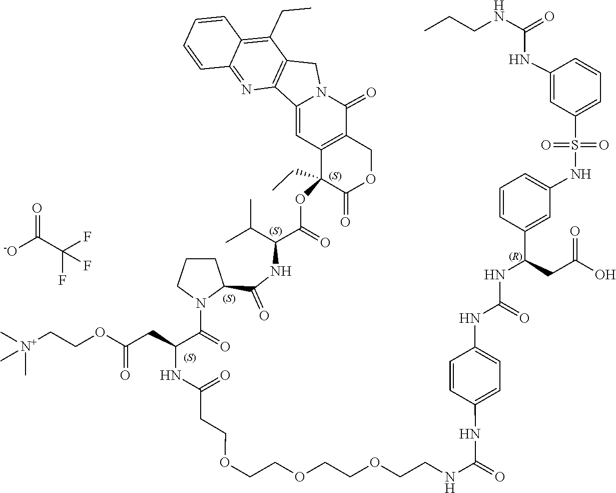

(14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N-trimethyl-12,16-dioxo-3,6,9,17-tetraoxa-13-azanonadecan-19-aminium trifluoroacetate trifluoroacetic acid (Intermediate 7) (15.0 mg, 12.5 μmol) was initially dissolved in DMF (5.0 mL). (3R)-3-{[(4-{[(4-nitrophenoxy) carbonyl]amino}phenyl) carbamoyl]amino}-3-[3-({3-[(propylcarbamoyl)amino]benzene-1-sulfonyl}amino) phenyl]propanoic acid (Building Block 2) (9.86 mg, 13.7 μmol) and DIEA (43 μl, 250 μmol) were added. The reaction was stirred for 30 min at rt. The reaction was evaporated to dryness and the residue was separated by prep. HPLC to give Compound C1 (2.27 mg, 100% 5 purity, 11%) as a beige foam. LC-MS: Rₜ=2.58 min; MS (ESIpos): m/z=1556 [M+H]⁺. ¹H-NMR (600 MHz, DMSO-d₆): δ [ppm]=12.51-12.15 (m, 1H), 10.26 (s, 1H), 8.77 (s, 1H), 8.47 (br d, 1H), 8.46 (br d, 1H), 8.36 (s, 1H), 8.33 (s, 1H), 8.30 (d, 1H), 8.12 (d, 1H), 8.06 (s, 1H), 7.88 (t, 1H), 7.75 (t, 1H), 7.40 (br d, 1H), 7.30-7.26 (m, 1H), 7.26-7.23 (m, 1H), 7.22-7.21 (m, 1H), 7.20 (s, 3H), 7.17-7.15 (m, 1H), 7.14 (d, 1H), 7.10 (s, 1H), 6.99 (d, 1H), 6.90 (br d, 1H), 6.64 (d, 1H), 6.22 (br t, 1H), 6.06 (br t, 1H), 5.50 (s, 2H), 5.40-5.30 (m, 2H), 5.03-4.97 (m, 2H), 4.61 (dd, 1H), 4.44-4.31 (m, 2H), 4.16 (t, 1H), 3.68-3.63 (m, 3H), 3.63-3.59 (m, 4H), 3.59-3.51 (m, 11H), 3.51-3.47 (m, 16H), 3.27-3.18 (m, 5H), 3.03 (s, 8H), 3.10-3.01 (m, 5H), 2.80-2.69 (m, 2H), 2.65-2.61 (m, 3H), 2.61-2.53 (m, 17H), 2.53-2.52 (m, 3H), 2.45-2.30 (m, 3H), 2.28-2.11 (m, 5H), 2.08-1.93 (m, 3H), 1.92-1.80 (m, 1H), 1.49-1.36 (m, 3H), 1.31 (t, 3H), 1.03-0.98 (m, 3H), 0.98-0.94 (m, 3H), 0.94-0.89 (m, 3H), 0.85 (t, 3H).

### Example C2: Preparation of (3R)-3-[({4-[({(9S)-9-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl} pyrrolidine-1-carbonyl]-2-methyl-7,11-dioxo-6,14,17,20-tetraoxa-2,10-diazadocosan-22-yl} carbamoyl)amino]phenyl}carbamoyl) amino]-3-[3-({3-[(propylcarbamoyl)amino]benzene-1-sulfonyl}amino)phenyl]propanoic acid (Compound C2)

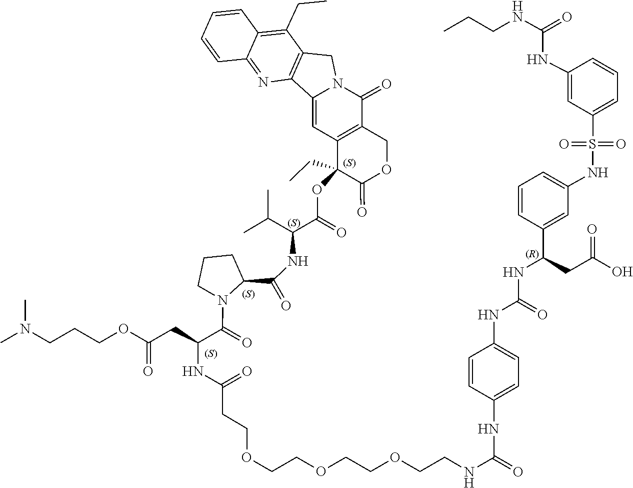

To a solution of trifluoroacetic acid 3-(dimethylamino)propyl (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-12-oxo-3,6,9-trioxa-13-azahexadecan-16-oate (Intermediate 9) (34.0 mg, 99% purity, 30.9 μmol) in DMF (8.0 mL) was added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (Building Block 2) (22.2 mg, 30.9 μmol) and DIEA (22 μl, 120 μmol). The reaction was stirred overnight at rt. It was concentrated in vacuo and the residue was purified by prep. HPLC, concentrated and lyophilized to give Compound C2 (31.0 mg, 99% purity, 64%) as a yellow foam. LC-MS: Rt=2.58 min; MS (ESIpos): m/z=1557 [M+H]⁺. ¹H-NMR (600 MHz, DMSO-d₆): δ [ppm]=12.42-12.17 (m, 1H), 10.26 (s, 1H), 9.28-9.18 (m, 1H), 8.77 (s, 1H), 8.44 (br d, 1H), 8.36 (s, 1H), 8.35-8.31 (m, 2H), 8.30 (br d, 1H), 8.13-8.08 (m, 1H), 8.07 (br s, 1H), 8.06 (s, 1H), 7.87 (t, 1H), 7.77-7.72 (m, 1H), 7.40 (br d, 1H), 7.30-7.26 (m, 1H), 7.26-7.23 (m, 1H), 7.20 (s, 4H), 7.17-7.14 (m, 1H), 7.14 (d, 1H), 6.99 (d, 1H), 6.90 (d, 1H), 6.64 (br d, 1H), 6.23 (t, 1H), 6.08-6.03 (m, 1H), 5.54-5.46 (m, 2H), 5.35 (br d, 2H), 5.04-4.95 (m, 2H), 4.68-4.61 (m, 1H), 4.20-4.15 (m, 1H), 4.11 (s, 1H), 4.06-3.97 (m, 2H), 3.72-3.64 (m, 2H), 3.62-3.56 (m, 3H), 3.51-3.49 (m, 3H), 3.49-3.46 (m, 5H), 3.46-3.40 (m, 19H), 3.27-3.15 (m, 5H), 3.08-2.98 (m, 4H), 2.78-2.72 (m, 4H), 2.71 (br d, 2H), 2.65-2.61 (m, 2H), 2.54 (s, 11H), 2.52-2.52 (m, 1H), 2.40-2.31 (m, 3H), 2.24-2.12 (m, 4H), 2.03-1.95 (m, 2H), 1.93-1.85 (m, 2H), 1.43 (sxt, 2H), 1.31 (t, 3H), 0.98 (d, 2H), 0.97-0.94 (m, 3H), 0.94-0.91 (m, 2H), 0.91-0.87 (m, 1H), 0.85 (t, 3H).

### Example C3: Preparation of (3R)-3-[({4-[({(10S)-10-[(2S)-2-{[(2S)-1-{[(4S)-4,11-Diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl} pyrrolidine-1-carbonyl]-2-methyl-8,12-dioxo-7,15,18,21-tetraoxa-2,11-diazatricosan-23-yl} carbamoyl)amino]phenyl}carbamoyl)amino]-3-[3-({3-[(propylcarbamoyl)amino]benzene-1-sulfonyl}amino)phenyl]propanoic acid (Compound C3)

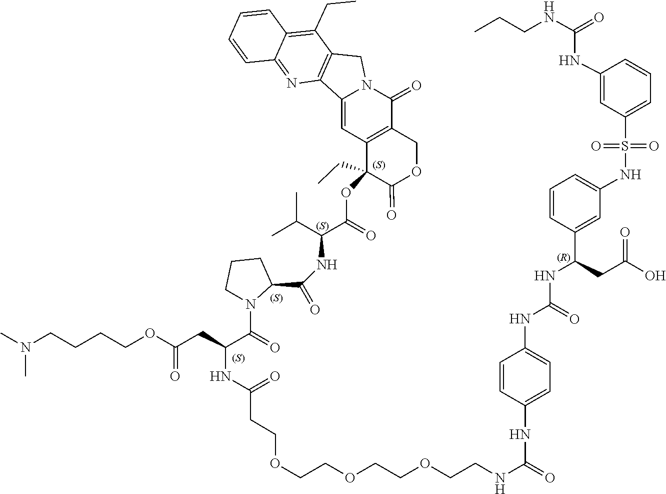

To a solution of trifluoroacetic acid 4-(dimethylamino)butyl (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-12-oxo-3,6,9-trioxa-13-azahexadecan-16-oate (Intermediate 8) (34.0 mg, 98% purity, 30.3 μmol) in DMF (8.0 mL) was added (3R)-3-{[(4-{[(4-nitrophenoxy)carbonyl]amino}phenyl)carbamoyl]amino}-3-[3-({3-[(propylcarbamoyl) amino]benzene-1-sulfonyl}amino)phenyl]propanoic acid (Building Block 2) (21.8 mg, 30.3 μmol) and DIEA (21 μl, 120 μmol). The reaction was stirred overnight at rt and then concentrated in vacuo. The residue was purified by prep. HPLC, concentrated and lyophilized to give Compound C3 (33.0 mg, 92% purity, 64%) as a yellow residue. LC-MS: Rₜ=2.61 min; MS (ESIpos): m/z=1570 [M+H]⁺. 1H-NMR (600 MHz, DMSO-d₆): δ [ppm]=10.27 (s, 1H), 8.77 (s, 1H), 8.43 (br d, 1H), 8.37-8.24 (m, 4H), 8.09 (d, 1H), 8.06 (s, 1H), 7.87 (t, 1H), 7.74 (t, 1H), 7.40 (br d, 1H), 7.29-7.22 (m, 2H), 7.20 (s, 4H), 7.18-7.14 (m, 3H), 7.01-6.97 (m, 1H), 6.90 (br d, 1H), 6.64 (br d, 1H), 6.23 (br s, 1H), 6.06 (br s, 1H), 5.50 (br d, 2H), 5.35 (br d, 2H), 4.99 (br dd, 2H), 4.71 (br d, 1H), 4.15 (br t, 2H), 4.04-3.92 (m, 3H), 3.76-3.58 (m, 12H), 3.48-3.45 (m, 11H), 3.42 (br t, 9H), 3.33-3.18 (m, 6H), 3.12-2.96 (m, 5H), 2.79-2.68 (m, 8H), 2.67-2.58 (m, 3H), 2.55-2.52 (m, 14H), 2.39-2.32 (m, 2H), 2.23-2.13 (m, 4H), 2.03-1.96 (m, 2H), 1.63-1.57 (m, 2H), 1.57-1.52 (m, 2H), 1.46-1.39 (m, 2H), 1.31 (t, 3H), 1.01-0.89 (m, 9H), 0.85 (t, 3H)

### Example C4: Preparation of disodium (3R,3′R)-3,3′-[{(14S)-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl] carbamoyl}pyrrolidine-1-carbonyl]-12,16-dioxo-3,6,9,17-tetraoxa-13-azahenicosane-1,21-diyl}bis(carbamoylazanediyl-4,1-phenylenecarbamoylazanediyl)]bis{3-[3-({3-[(propylcarbamoyl)amino]benzene-1-sulfonyl}amino)phenyl]propanoate} (Compound C4)

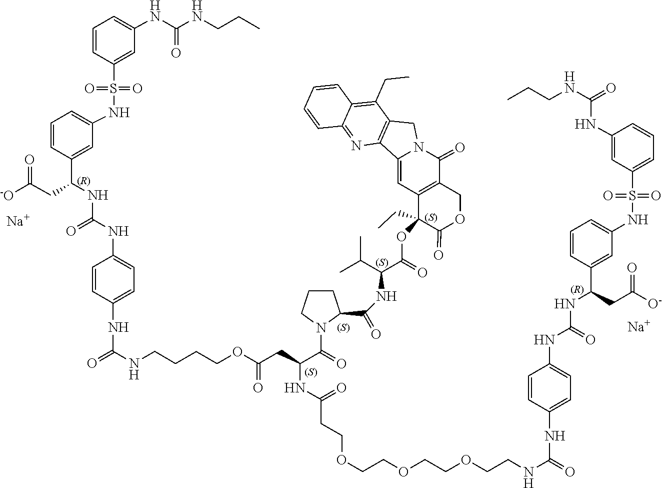

4-aminobutyl (14S)-1-amino-14-{[(2S)-2-{[(2S)-1-{[(4S)-4,11-diethyl-3,14-dioxo-3,4,12,14-tetrahydro-1H-pyrano[3′,4′:6,7] indolizino [1,2-b]quinolin-4-yl]oxy}-3-methyl-1-oxobutan-2-yl]carbamoyl} pyrrolidin-1-yl]carbonyl}-12-oxo-3,6,9-trioxa-13-azahexadecan-16-oate trifluoroacetate (Intermediate 10) (47 mg, 39.5 μmol) was dissolved in DMF (10 mL). (3R)-3-{[(4-{[(4-nitrophenoxy)carbonyl]amino}phenyl)carbamoyl]amino}-3-{3-[({3-[(propylcarbamoyl)amino]phenyl}sulfonyl)amino]phenyl}propanoic acid (Building Block 2) (62.5 mg, 86.9 μmol) and DIEA (137.6 μL, 789.9 μmol, ) were added. The reaction was stirred for 30 min at rt and then evaporated to dryness. The compound was sparingly soluble but was dissolved in DMSO for prep. HPLC. During prep. HPLC the flow rate had to be reduced due to the low solubility and a beige foam was obtained. This residue (20.2 mg, 9.51 μmol) was dissolved in dioxane/water (1:1, 10 mL). A sodium hydroxide solution (190 μl, 0.10 M, 19 μmol) was added. The solution was freeze-dried to give Compound C4 (17.0 mg, 100% purity, 57%) as an almost colorless foam. LC-MS: Rₜ=3.35 min; MS (ESIpos): m/z=2119 [M+H]⁺. ¹H-NMR (700 MHz, DMSO-d₆): δ [ppm]=10.27 (s, 1H), 9.32 (br s, 1H), 8.47-8.41 (m, 1H), 8.39 (s, 1H), 8.36 (s, 1H), 8.29 (br d, 1H), 8.28-8.25 (m, 1H), 8.19-8.13 (m, 1H), 8.08 (br d, 1H), 8.07 (s, 1H), 7.86 (br t, 1H), 7.74 (t, 1H), 7.40 (br d, 1H), 7.28 (t, 1H), 7.25 (d, 1H), 7.20 (s, 4H), 7.17 (s, 1H), 7.15 (s, 1H), 7.15 (d, 2H), 6.99 (br d, 1H), 6.90 (br d, 1H), 6.67 (br d, 1H), 6.29 (br t, 1H), 6.09 (br s, 1H), 5.50 (br s, 2H), 5.38-5.30 (m, 2H), 5.02-4.97 (m, 2H), 4.72 (br d, 1H), 4.15 (br t, 1H), 4.02-3.95 (m, 2H), 3.71 (br d, 1H), 3.67 (br s, 1H), 3.60 (br t, 2H), 3.51 (s, 4H), 3.48 (br d, 4H), 3.44-3.40 (m, 5H), 3.25-3.18 (m, 5H), 3.05-3.01 (m, 2H), 3.00 (br s, 2H), 2.80-2.74 (m, 1H), 2.73 (br s, 1H), 2.72 (br s, 4H), 2.63 (br d, 1H), 2.35 (br s, 2H), 2.23-2.19 (m, 2H), 2.19-2.14 (m, 2H), 2.00 (br d, 2H), 1.89 (br s, 1H), 1.81-1.72 (m, 1H), 1.60 (br d, 2H), 1.55 (br d, 2H), 1.46-1.40 (m, 2H), 1.31 (br t, 3H), 1.08 (br t, 1H), 0.98-0.94 (m, 6H), 0.94-0.88 (m, 3H), 0.85 (br t, 3H).

### Example C5: Preparation of (16S)-1-[4-({[(1R)-2-carboxy-1-{3-[({3 [(propylcarbamoyl)amino]phenyl}sulfonyl)amino]phenyl}ethyl]carbamoyl}amino)anilino]-16-{[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidin-1-yl]carbonyl}-N,N,N-trimethyl-1,14,18-trioxo-5,8,11,19-tetraoxa-2,15-diazadocosan-22-aminium trifluoroacetate (Compound C5)

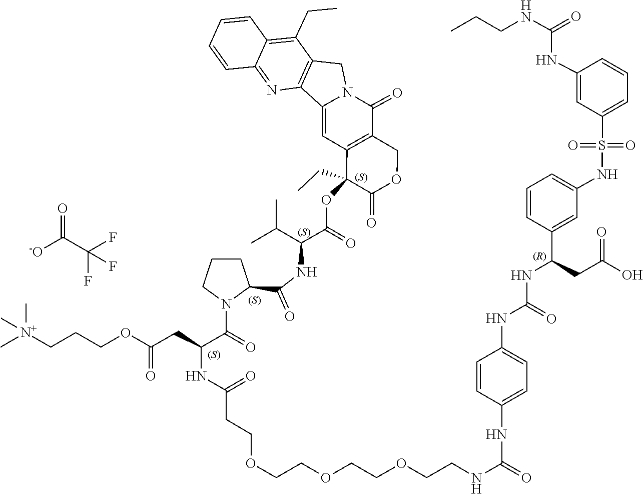

To a solution of (14S)-1-amino-14-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N-trimethyl-12,16-dioxo-3,6,9,17-tetraoxa-13-azaicosan-20-aminium trifluoroacetate trifluoroacetic acid (Intermediate 25) (20.0 mg, 77% purity, 12.6 μmol) in DMF (5.0 ml) were added (3R)-3-{[(4-{[(4-nitrophenoxy)carbonyl] amino} phenyl)carbamoyl]amino}-3-[3-({3-[(propylcarbamoyl)amino]benzene-1-sulfonyl}amino)phenyl]propanoic acid (Building Block 2) (18.2 mg, 25.2 μmol) and N,N-diisopropylethylamine (11 μl, 63 μmol). The reaction was then stirred for 30 min at RT, concentrated in vacuo and purified by prep. HPLC. The product was lyophilized to give Compound C5 (13.0 mg, 100% purity, 61% yield) as an amorphous residue. LC-MS: Rₜ=3.40 min; MS (ESIpos): m/z=1570 [M+H]⁺. ¹H NMR (600 MHz, DMSO-d₆) δ ppm 0.835-0.874 (m, 1H) 0.895-0.941 (m, 3H) 0.954-0.999 (m, 5H) 1.307 (t, J=7.63 Hz, 3H) 1.395-1.458 (m, 2H) 1.824-1.927 (m, 1H) 1.945-2.061 (m, 4H) 2.108-2.240 (m, 5H) 2.340-2.380 (m, 2H) 2.519-2.537 (m, 1H) 2.541 (s, 15H) 2.619-2.651 (m, 2H) 2.712-2.857 (m, 2H) 2.966 (s, 6H) 2.971-3.089 (m, 5H) 3.188-3.252 (m, 5H) 3.268-3.309 (m, 2H) 3.318-3.448 (m, 16H) 3.475-3.476 (m, 2H) 3.499-3.507 (m, 7H) 3.517-3.647 (m, 13H) 3.664-3.702 (m, 2H) 3.964-4.055 (m, 2H) 4.152 (t, J=7.82 Hz, 1H) 4.631-4.678 (m, 1H) 4.968-5.043 (m, 2H) 5.309-5.390 (m, 1H) 5.350 (br d, J=7.82 Hz, 1H) 5.470-5.519 (m, 2H) 6.035-6.077 (m, 1H) 6.202-6.231 (m, 1H) 6.634 (br d, J=8.22 Hz, 1H) 6.895 (d, J=8.19 Hz, 1H) 6.971-7.008 (m, 1H) 7.121-7.193 (m, 3H) 7.201 (s, 3H) 7.214 (br s, 1H) 7.232-7.294 (m, 2H) 7.406 (d, J=7.95 Hz, 1H) 7.748 (t, J=7.73 Hz, 1H) 7.855-7.890 (m, 1H) 8.057 (s, 1H) 8.298 (d, J=8.51 Hz, 1H) 8.325 (s, 1H) 8.361 (d, J=7.47 Hz, 2H) 8.355 (s, 1H) 8.446 (d, J=8.27 Hz, 1H) 8.765 (s, 1H) 10.265 (s, 1H) 12.159-12.417 (m, 1H).

### Example C6: Preparation of sodium (3R)-3-[({4-[({(10S)-10-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2-methyl-8,12-dioxo-7,15,18,21-tetraoxa-2,11-diazatricosan-23-yl}carbamoyl)amino]phenyl}carbamoyl)amino]-3-[3-({3-[(propylcarbamoyl)amino]benzene-1-sulfonyl}amino)phenyl]propanoate (Compound C6)

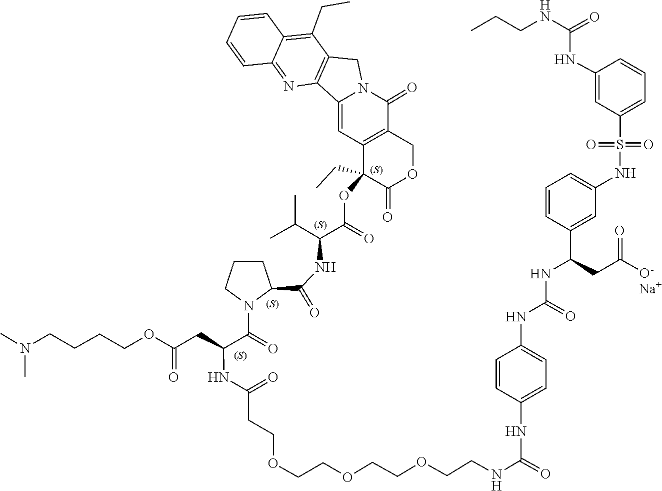

To a solution of (3R)-3-[({4-[({(10S)-10-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-2-methyl-8,12-dioxo-7,15,18,21-tetraoxa-2,11-diazatricosan-23-yl}carbamoyl)amino]phenyl}carbamoyl)amino]-3-[3-({3-[(propylcarbamoyl)amino]benzene-1-sulfonyl}amino)phenyl]propanoic acid (Compound C3) (59.0 mg, 37.6 μmol) in dioxane/water (1:1, 10 mL) was added a sodium hydroxide solution (75.2 μl, 1 M, 75.2 μmol). The solution was freeze-dried to give Compound C6 (59.0 mg, 97% purity, 96%) as an almost colorless foam. LC-MS: Rₜ=3.44 min; MS (ESIpos): m/z=1570 [M+H]⁺. (Method 3): ¹H-NMR (600 MHz, DMSO-d₆) δ[ppm]: 0.068 (0.74), 0.812 (2.86), 0.825 (6.06), 0.837 (3.17), 0.874 (0.78), 0.887 (1.40), 0.891 (1.77), 0.904 (3.23), 0.916 (1.67), 0.926 (0.96), 0.934 (1.28), 0.955 (4.77), 0.966 (4.75), 1.036 (0.48), 1.048 (0.72), 1.060 (0.41), 1.236 (0.66), 1.295 (2.04), 1.308 (4.20), 1.321 (2.19), 1.340 (2.24), 1.352 (2.87), 1.364 (2.43), 1.376 (1.08), 1.482 (0.71), 1.496 (0.88), 1.508 (0.73), 1.914 (0.52), 2.006 (0.48), 2.040 (16.00), 2.054 (3.74), 2.068 (1.12), 2.085 (1.19), 2.093 (1.43), 2.105 (2.00), 2.117 (1.34), 2.136 (0.63), 2.148 (0.91), 2.159 (1.12), 2.171 (1.22), 2.183 (1.39), 2.194 (1.20), 2.206 (0.76), 2.335 (0.92), 2.345 (1.82), 2.356 (0.94), 2.516 (2.32), 2.653 (0.41), 2.755 (0.56), 2.767 (0.53), 2.782 (0.54), 2.905 (0.72), 2.915 (1.52), 2.925 (1.64), 2.937 (0.69), 3.030 (0.75), 3.071 (1.43), 3.199 (1.62), 3.208 (1.96), 3.218 (1.78), 3.232 (1.30), 3.404 (1.87), 3.414 (2.71), 3.423 (1.81), 3.472 (3.09), 3.479 (3.40), 3.483 (3.22), 3.504 (11.19), 3.568 (0.83), 3.581 (1.14), 3.592 (2.02), 3.603 (1.13), 3.697 (0.53), 3.743 (0.64), 3.955 (1.10), 3.963 (1.07), 3.974 (0.58), 4.108 (0.62), 4.121 (0.86), 4.135 (0.65), 4.786 (0.49), 4.795 (0.57), 4.948 (0.62), 4.961 (0.83), 4.974 (0.70), 5.334 (1.71), 5.346 (1.38), 5.492 (2.27), 6.067 (0.76), 7.173 (1.52), 7.188 (2.00), 7.216 (2.66), 7.255 (2.77), 7.270 (1.90), 7.358 (0.41), 7.527 (0.48), 7.726 (0.52), 7.738 (0.87), 7.752 (0.60), 7.847 (0.78), 7.859 (1.19), 7.872 (0.82), 8.058 (1.08), 8.071 (0.93), 8.139 (0.80), 8.153 (0.84), 8.282 (1.11), 8.297 (1.10), 8.316 (1.22), 8.407 (0.71), 8.421 (0.77).

### Example C7: Preparation of 4-aminobutyl (16S)-1-{4-[({(1R)-3-(4-aminobutoxy)-3-oxo-1-[3-({3-[(propylcarbamoyl)amino]benzene-1-sulfonyl}amino)phenyl]propyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-1,14-dioxo-5,8,11-trioxa-2,15-diazaoctadecan-18-oate·trifluoroacetic acid (½) (Compound C7)

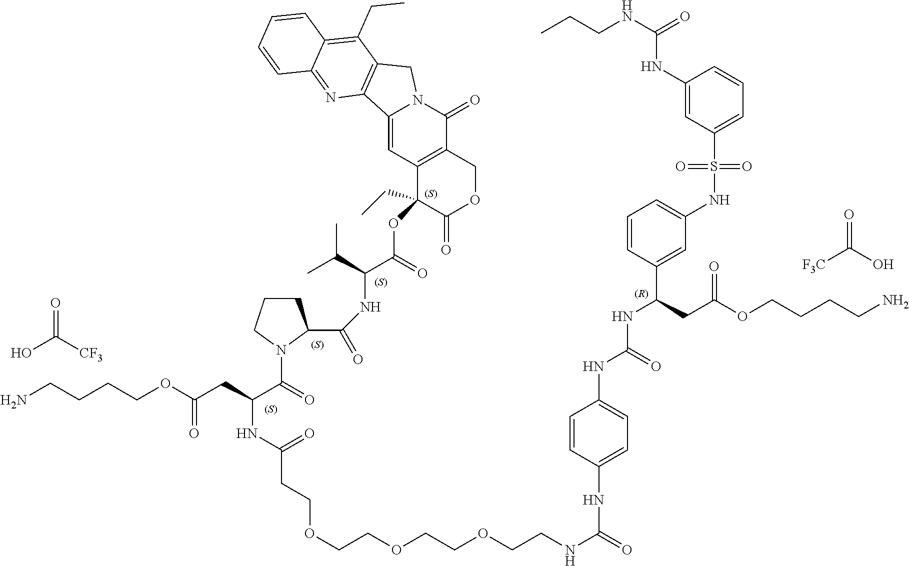

Step 1: To solution of (4S)-4,11-diethyl-3,14-dioxo-3,4,12,14-tetrahydro-1H-pyrano[3′,4′:6,7]indolizino[1,2-b]quinolin-4-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-valinate (30.0 mg, 20.4 μmol) (synthesis described in described in WO2020/094471) in DCM (10 ml), were added tert-butyl (4-hydroxybutyl)carbamate (8.66 mg, 98% purity, 44.8 μmol), EDCI (5.86 mg, 30.6 μmol) and DMAP (4.98 mg, 40.8 μmol). The mixture was overnight stirred at rt and then concentrated under vacuum. The residue was purified over preparative HPLC and lyophilized to afford 4-[(tert-butoxycarbonyl)amino]butyl (16S)-1-{4-[({(1R)-3-{4-[(tert-butoxycarbonyl)amino]butoxy}-3-oxo-1-[3-({3-[(propylcarbamoyl)amino]benzene-1-sulfonyl}amino)phenyl]propyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-1,14-dioxo-5,8,11-trioxa-2,15-diazaoctadecan-18-oate (16.5 mg, 93% purity, 51% yield). LC-MS (Method 6): Rₜ=4.11 min; MS (ESIpos): m/z=807 [M+2H]²⁺.
Step 2: To a solution of 4-[(tert-butoxycarbonyl)amino]butyl (16S)-1-{4-[({(1R)-3-{4-[(tert-butoxycarbonyl)amino]butoxy}-3-oxo-1-[3-({3-[(propylcarbamoyl)amino]benzene-1-sulfonyl}amino)phenyl]propyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-1,14-dioxo-5,8,11-trioxa-2,15-diazaoctadecan-18-oate (16.5 mg, 93% purity, 8.42 μmol) in DCM (5 mL), was added TFA (1 mL). The reaction mixture was then stirred at room temperature for 1 h and concentrated in vacuo. The residue was dissolved in ACN/H₂O and lyophilized to afford Compound C7 (15.0 mg, 97% purity, 94%) as a yellow solid. LC-MS (Method 6): Rₜ=2.22 min; MS (ESIpos): m/z=1613 [M+H]⁺.

### Example C8: Preparation of 3-aminopropyl (16S)-1-{4-[({(1R)-3-(3-aminopropoxy)-3-oxo-1-[3-({3-[(propylcarbamoyl)amino]benzene-1-sulfonyl}amino)phenyl]propyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-1,14-dioxo-5,8,11-trioxa-2,15-diazaoctadecan-18-oate·trifluoroacetic acid (½) (Compound C8)

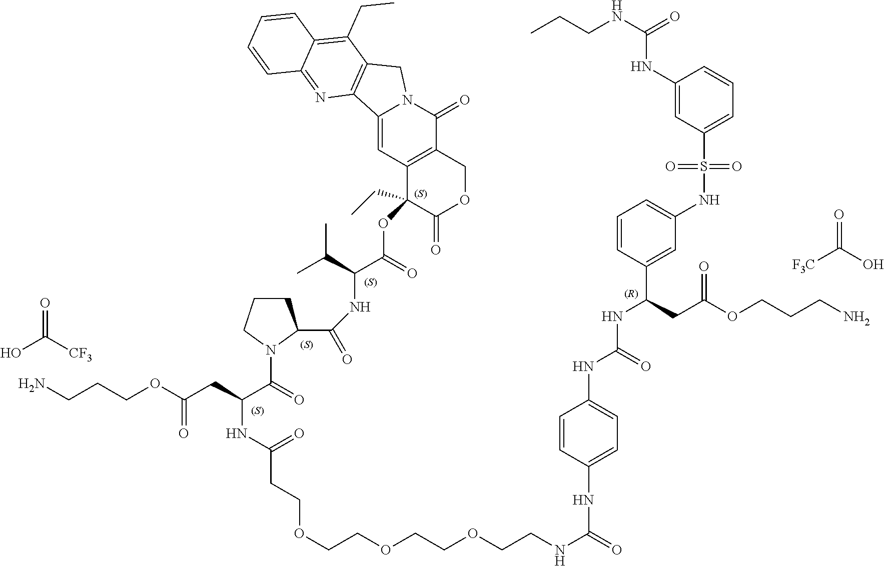

Step 1: To solution of (4S)-4,11-diethyl-3,14-dioxo-3,4,12,14-tetrahydro-1H-pyrano[3′,4′:6,7]indolizino[1,2-b]quinolin-4-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-valinate (30.0 mg, 20.4 μmol) (synthesis described in described in WO2020/094471) in DCM (10 ml), were added tert-butyl (3-hydroxypropyl)carbamate (8.10 mg, 97% purity, 44.8 μmol), EDCI (7.82 mg, 40.8 μmol) and DMAP (3.74 mg, 30.6 μmol). The mixture was overnight stirred at rt and then concentrated under vacuum. The residue was purified over preparative HPLC and lyophilized to afford 3-[(tert-butoxycarbonyl)amino]propyl (16S)-1-{4-[({(1R)-3-{3-[(tert-butoxycarbonyl)amino]propoxy}-3-oxo-1-[3-({3-[(propylcarbamoyl)amino]benzene-1-sulfonyl}amino)phenyl]propyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-1,14-dioxo-5,8,11-trioxa-2,15-diazaoctadecan-18-oate (7.0 mg, 80% purity, 15% yield) as a yellow solid. LC-MS (Method 6): Rₜ=4.0 min; MS (ESIpos): m/z=793 [M+2H]²¹.
Step 2: To a solution of 3-[(tert-butoxycarbonyl)amino]propyl (16S)-1-{4-[({(1R)-3-{3-[(tert-butoxycarbonyl)amino]propoxy}-3-oxo-1-[3-({3-[(propylcarbamoyl)amino]benzene-1-sulfonyl}amino)phenyl]propyl}carbamoyl)amino]anilino}-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-1,14-dioxo-5,8,11-trioxa-2,15-diazaoctadecan-18-oate (7.00 mg, 80% purity, 3.14 μmol) in DCM (5 mL), was added TFA (1 mL). The reaction mixture was then stirred at room temperature for 45 min and then purified over preparative HPLC and lyophilized to afford Compound C8 (5.5 mg, 100% purity, 97%) as a yellow foam. LC-MS: Rₜ=2.20 min; MS (ESIpos): m/z=1585 [M+H]⁺. (Method 6)

### Example C9: Preparation of 3 (16S)-16-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-N,N,N-trimethyl-1,14,18-trioxo-1-{4-[({(1R)-3-oxo-1-[3-({3-[(propylcarbamoyl)amino]benzene-1-sulfonyl}amino)phenyl]-3-[2-(trimethylazaniumyl)ethoxy]propyl}carbamoyl)amino]anilino}-5,8,11,19-tetraoxa-2,15-diazahenicosan-21-aminium dichloride (Compound C9)

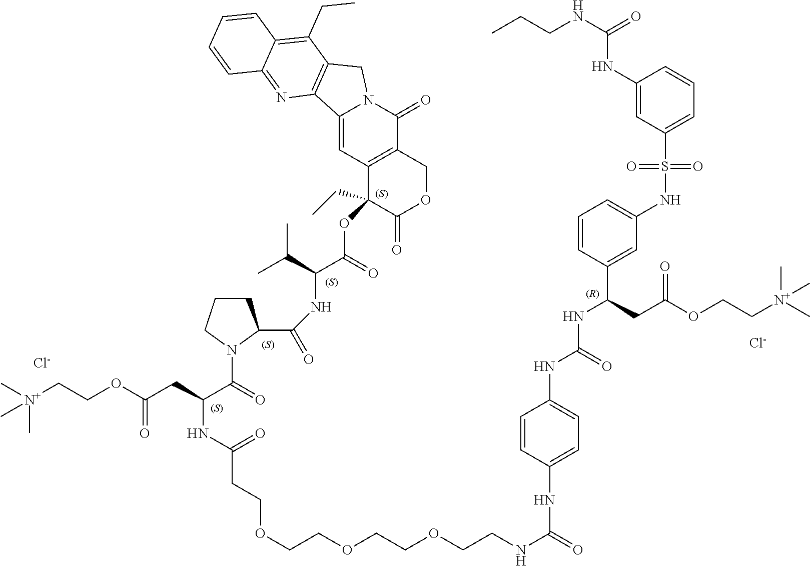

To a solution (4S)-4,11-diethyl-3,14-dioxo-3,4,12,14-tetrahydro-1H-pyrano[3′,4′:6,7]indolizino[1,2-b]quinolin-4-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-valinate (30.0 mg, 20.4 μmol) ) (synthesis described in described in WO2020/094471) and 2-hydroxy-N,N,N-trimethylethan-1-aminium chloride (29.0 mg, 98% purity, 204 μmol) in DMF (10 mL), were added EDCI (15.6 mg, 81.5 μmol) and DMAP (9.96 mg, 81.5 μmol). The reaction mixture was then stirred at room temperature for 72 h and concentrated in vacuo. The residue was then purified over preparative HPLC and lyophilized to afford Compound C9 (5.5 mg, 100% purity, 97%) as a yellow solid. LC-MS: Rₜ=2.19 min; MS (ESIpos): m/z=821 [M+2H]²⁺. (Method 6)

### Example C10: Preparation of disodium (4S)-4,11-diethyl-3,14-dioxo-3,4,12,14-tetrahydro-1H-pyrano[3′,4′:6,7]indolizino[1,2-b]quinolin-4-yl 1-[(2S)-2-(carboxylatomethyl)-17-{4-[({(1R)-2-carboxylato-1-[3-({3-[(propylcarbamoyl)amino]benzene-1-sulfonyl}amino)phenyl]ethyl}carbamoyl)amino]anilino}-4,17-dioxo-7,10,13-trioxa-3,16-diazaheptadecanan-1-oyl]-L-prolyl-L-alaninate (Compound C10)

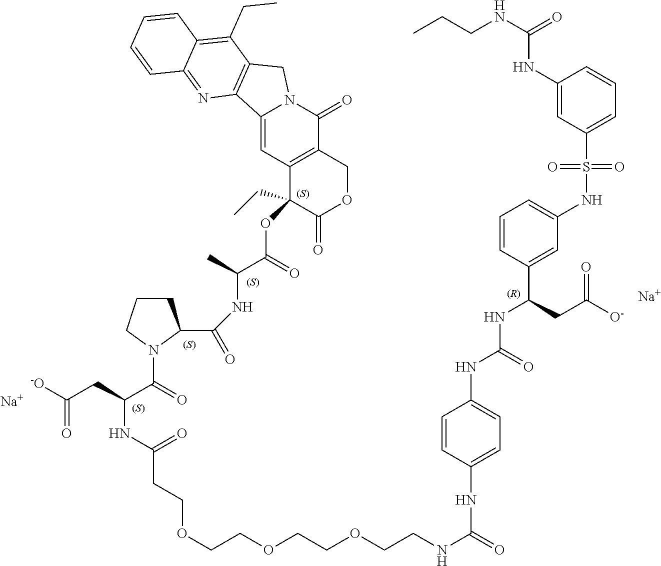

Step 1: To a solution of (4S)-4,11-diethyl-3,14-dioxo-3,4,12,14-tetrahydro-1H-pyrano[3′,4′:6,7]indolizino[1,2-b]quinolin-4-yl N-(3-{2-[12-(2-aminoethoxy)ethoxy]ethoxy}propanoyl)-L-alpha-aspartyl-L-prolyl-L-alaninate trifluoroacetic acid (1/1) (30.0 mg, 94% purity, 28.9 μmol) (Intermediate 65) in DMF (5 mL) were added (3R)-3-{[(4-{[(4-nitrophenoxy) carbonyl]amino}phenyl)carbamoyl]amino}1-3-{3-[({3-[(propylcarbamoyl)amino]phenyl} sulfonyl)amino]phenyl}propanoic acid (Building Block 2) (24.0 mg, 9500 purity, 31.8 μmol) and DIEA (25 μl, 140 μmol). The reaction was stirred for 2 h at rt. The reaction was evaporated to dryness and the residue was purified by preparative HPLC to yield (4S)-4,11-diethyl -3,14-dioxo-3,4,12,14-tetrahydro-1H-pyrano[3′,4′:6,7]indolizino[1,2-b]quinolin-4-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-alaninate (25.8 mg, 100% purity, 62% yield) as an amorphous residue. LC-MS (Method 3): Rₜ=3.83 min; MS (ESIpos): m/z=1444 [M+H]⁺.
Step 2: To a solution of (4S)-4,11-diethyl-3,14-dioxo-3,4,12,14-tetrahydro-1H-pyrano[3′,4′:6,7]indolizino[1,2-b]quinolin-4-yl N-(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)-L-alpha-aspartyl-L-prolyl-L-alaninate (25.3 mg, 100% purity, 17.5 μmol) in dioxane/water (1:1, 10 mL) was added a sodium hydroxide solution (35 μl, 1 M, 35 μmol). The solution was freeze-dried to give Compound C10 (25.7 mg, 97% purity, 96% yield) as a colorless foam. LC-MS (Method 3): Rₜ=3.85 min; MS (ESIpos): m/z=1443 [M+H]⁺. ¹H-NMR (400 MHz, DMSO-d₆) δ[ppm]: 0.804 (5.67), 0.823 (12.86), 0.841 (6.60), 0.875 (3.76), 0.893 (7.97), 0.912 (4.13), 1.231 (1.39), 1.288 (4.04), 1.306 (8.43), 1.326 (4.54), 1.350 (4.34), 1.368 (4.50), 1.385 (8.54), 1.403 (7.06), 1.446 (0.72), 1.464 (0.72), 1.936 (2.88), 2.064 (1.10), 2.175 (2.39), 2.190 (3.04), 2.211 (3.54), 2.327 (1.27), 2.366 (1.21), 2.393 (1.33), 2.410 (1.45), 2.430 (1.32), 2.575 (1.90), 2.629 (1.18), 2.669 (1.57), 2.709 (0.83), 2.898 (1.72), 2.913 (3.52), 2.930 (3.60), 2.946 (1.68), 3.175 (4.21), 3.188 (5.28), 3.207 (4.71), 3.226 (4.71), 3.457 (6.46), 3.471 (7.74), 3.497 (16.00), 3.540 (2.97), 3.569 (2.05), 3.598 (1.43), 3.619 (1.61), 3.632 (1.48), 3.817 (1.15), 3.837 (1.22), 3.950 (1.10), 4.129 (1.30), 4.147 (1.74), 4.164 (1.24), 4.645 (0.45), 4.776 (2.05), 4.794 (2.73), 4.960 (1.40), 5.276 (0.56), 5.330 (4.21), 5.378 (0.56), 5.462 (7.14), 6.628 (2.05), 6.648 (2.09), 6.923 (2.79), 6.942 (2.86), 7.034 (2.14), 7.053 (3.31), 7.072 (1.36), 7.113 (0.61), 7.163 (2.20), 7.182 (3.13), 7.199 (3.08), 7.222 (6.88), 7.242 (7.43), 7.269 (3.34), 7.289 (3.71), 7.310 (2.48), 7.338 (1.59), 7.419 (5.29), 7.492 (3.68), 7.680 (1.13), 7.698 (1.99), 7.715 (1.46), 7.833 (1.39), 7.866 (4.34), 8.068 (2.76), 8.090 (2.69), 8.116 (1.76), 8.176 (2.20), 8.196 (2.07), 8.255 (2.36), 8.275 (2.20), 8.772 (1.88), 8.813 (3.21), 8.959 (1.35), 9.207 (1.46), 11.250 (2.87).

### Example D1: Preparation of (4S)-4,11-diethyl-3,14-dioxo-3,4,12,14-tetrahydro-1H-pyrano[3′,4′:6,7]indolizino[1,2-b]quinolin-4-yl N-{2-[{2-[{2-[(N-{2-[{2-[{2-[({4-[({(1R)-2-carboxy-1-[3-({3-[(propylcarbamoyl) amino]benzene-1-sulfonyl}amino) phenyl]ethyl}carbamoyl)amino] phenyl}carbamoyl)amino]ethyl} (methyl)amino]ethyl}(methyl)amino]ethyl}-N-methylglycyl)amino]ethyl} (methyl)amino]ethyl}(methyl) amino]ethyl}-N-methylglycyl-L-asparaginyl-L-prolyl-L-valinate (Compound D1)

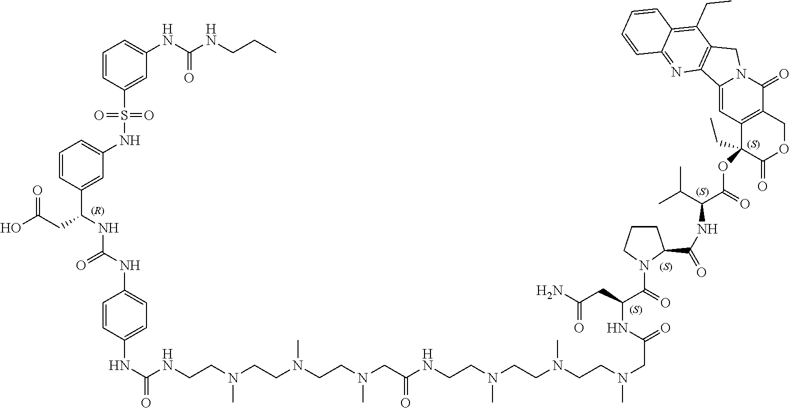

Trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[{2-[(N-{2-[{2-[(2-aminoethyl) (methyl)amino]ethyl} (methyl) amino]ethyl}-N-methylglycyl) amino]ethyl}(methyl) amino]ethyl}(methyl) amino]ethyl}-N-methylglycyl-L-asparaginyl-L-prolyl-L-valinate (1/1) (Intermediate 15) (16.0 mg, 88% purity, 11.2 μmol) was dissolved in DMF (4.0 mL). (3R)-3-{[(4-{[(4-nitrophenoxy)carbonyl] amino} phenyl)carbamoyl] amino}-3-[3-({3-[(propylcarbamoyl)amino]benzene-1-sulfonyl}amino)phenyl]propanoic acid (Building Block 2) (8.86 mg, 12.3 μmol) and DIEA (39 μl, 220 μmol) were added. The reaction was stirred for 30 min at RT, then the reaction was concentrated in vacuo. The residue was separated by prep. HPLC to give Compound D1 (10.7 mg, 92% purity, 51% yield) as a yellow foam. LC-MS: Rₜ=2.49 min; MS (ESIpos): m/z=1724 [M+H]⁺. ¹H NMR (600 MHz, DMSO-d₆) δ ppm 0.856 (t, J=7.43 Hz, 4H) 0.893-0.930 (m, 4H) 0.930-0.974 (m, 7H) 1.311 (t, J=7.63 Hz, 4H) 1.361-1.495 (m, 3H) 1.894-2.057 (m, 5H) 2.137-2.266 (m, 7H) 2.360 (br s, 7H) 2.412-2.470 (m, 2H) 2.517-2.594 (m, 4H) 2.542 (s, 10H) 2.608-2.670 (m, 5H) 2.722 (br s, 3H) 2.730-2.765 (m, 4H) 2.765-2.803 (m, 4H) 2.818-2.881 (m, 11H) 2.881-2.926 (m, 4H) 3.011-3.061 (m, 4H) 3.132-3.207 (m, 9H) 3.207-3.266 (m, 7H) 3.296-3.356 (m, 5H) 3.376-3.475 (m, 10H) 3.475-3.525 (m, 7H) 3.526-3.701 (m, 28H) 3.739-3.799 (m, 9H) 4.096 (br t, J=8.12 Hz, 2H) 4.831-4.874 (m, 1H) 4.931-5.045 (m, 3H) 5.306-5.396 (m, 3H) 5.496 (s, 2H) 6.349 (s, 1H) 6.650 (br d, J=3.52 Hz, 1H) 6.757 (br d, J=8.22 Hz, 1H) 6.860-6.905 (m, 1H) 6.986 (br d, J=7.43 Hz, 1H) 7.012 (br d, J=1.17 Hz, 1H) 7.123-7.156 (m, 1H) 7.166 (d, J=1.76 Hz, 2H) 7.214-7.248 (m, 4H) 7.248-7.260 (m, 2H) 7.260-7.301 (m, 2H) 7.350-7.464 (m, 2H) 7.544 (br s, 2H) 7.717-7.768 (m, 1H) 7.796-7.903 (m, 2H) 8.030 (d, J=8.41 Hz, 1H) 8.082 (t, J=1.96 Hz, 1H) 8.247-8.340 (m, 3H) 8.479 (s, 1H) 8.757-8.798 (m, 1H) 8.798-8.856 (m, 1H) 8.877 (s, 2H) 8.916-9.003 (m, 1H) 10.265 (s, 1H).

### Example D2: Preparation of (4S)-4,11-diethyl-3,14-dioxo-3,4,12,14-tetrahydro-1H-pyrano[3′,4′:6,7]indolizino[1,2b] quinolin-4-yl N-{2-[{2-[{2-[(N-{2-[{2-[{2-[({4-[({(1S)-2-carboxy-1-[3-({3-[(propylcarbamoyl) amino] benzene-1-sulfonyl}amino)phenyl]ethyl}carbamoyl)amino]phenyl} carbamoyl) amino]ethyl}(methyl)amino]ethyl}(methyl)amino]ethyl}-N-methylglycyl) amino]ethyl}(methyl) amino]ethyl}(methyl)amino]ethyl}-N-methylglycyl-L-asparaginyl-L-prolyl-L-valinate (Compound D2)

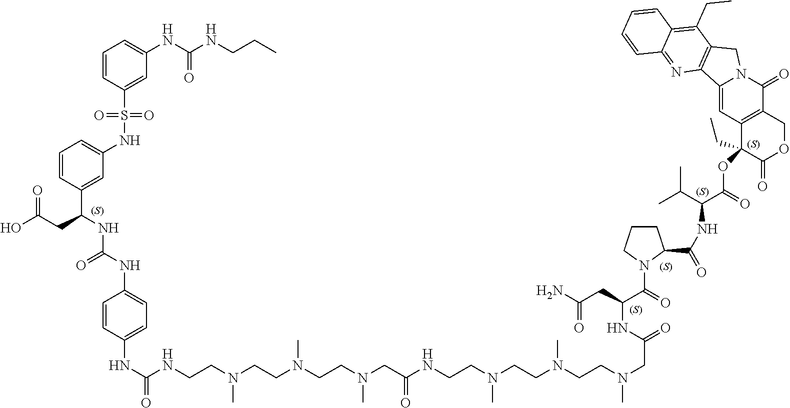

Trifluoroacetic acid-(4S)-4,11-diethyl-3,14-dioxo-3,4,12,14-tetrahydro-1H-pyrano[3′,4′:6,7] indolizino[1,2-b]quinolin-4-yl N-{2-[{2-[{2-[(N-{2-[{2-[(2-aminoethyl) (methyl)amino]ethyl} (methyl)amino]ethyl}-N-methylglycyl)amino]ethyl}(methyl) amino]ethyl} (methyl) amino]ethyl}-N-methylglycyl-L-asparaginyl-L-prolyl-L-valinate (Intermediate 15) (1/1) (10.0 mg, 7.95 μmol) was dissolved in DMF (3.0 mL). (3S)-3-{[(4-{[(4-nitrophenoxy) carbonyl]amino}phenyl)carbamoyl]amino}-3-[3-({3-[(propylcarbamoyl)amino]benzene-1-sulfonyl}amino)phenyl]propanoic acid (Building Block 11) (6.30 mg, 8.75 μmol) and DIEA (28 μl, 160 μmol) were added. The reaction was stirred for 30 min at RT. Then the reaction was concentrated in vacuo. The residue was separated three times by prep. HPLC (dissolved in pure water for loading onto the column) to give Compound D2 (1.21 mg, 100% purity, 9% yield) as a yellow foam. LC-MS: Rₜ=1.84 min; MS (ESIpos): m/z=1724 [M+H]⁺.

### Example D3: Preparation of disodium N-({4-[({(1R)-2-carboxylato-1-[3-({3-[(propylcarbamoyl)amino]benzene-1-sulfonyl}amino)phenyl]ethyl}carbamoyl)amino]phenyl}carbamoyl)-N-methylglycyl-N-methylglycyl-N-methylglycyl-N-methylglycyl-N-methylglycyl-N-methylglycyl-N-methylglycyl-N-{(2S)-3-carboxylato-1-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidin-1-yl]-1-oxopropan-2-yl}-N2-methylglycinamide (Compound D3)

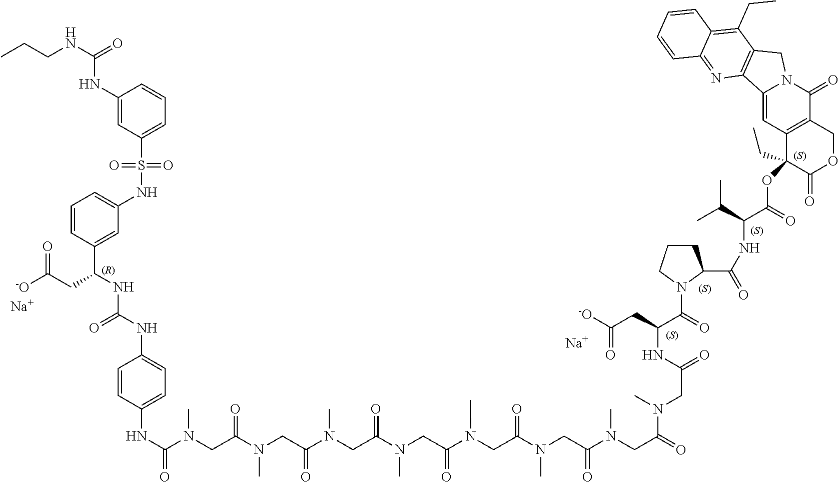

Step 1: To a solution of (4S)-4,11-diethyl-3,14-dioxo-3,4,12,14-tetrahydro-1H-pyrano[3′,4′:6,7]indolizino[1,2-b]quinolin-4-yl N-methylglycyl-N-methylglycyl-N-methylglycyl-N-methylglycyl-N-methylglycyl-N-methylglycyl-N-methylglycyl-N-methylglycyl-L-alpha-aspartyl-L-prolyl-L- valinate·trifluoroacetic acid (1/1) (56.0 mg, 97% purity, 39.8 μmol) (Intermediate 53) in DMF (10 ml), were added (3R)-3-{[(4-{[(4-nitrophenoxy)carbonyl]amino}phenyl)carbamoyl]amino}-3-[3-({3-[(propylcarbamoyl)amino]benzene-1-sulfonyl}amino)phenyl]propanoic acid (33.1 mg, 95% purity, 43.7 μmol) (Building Block 2) and DIEA (35 μl, 200 μmol). The mixture was stirred at rt for 15 min and then concentrated under reduced pressure. The residue was purified over preparative HPLC and freeze dried to afford (4S)-4,11-diethyl-3,14-dioxo-3,4,12,14-tetrahydro-1H-pyrano[3′,4′:6,7]indolizino[1,2-b]quinolin-4-yl N-({4-[({(1R)-2-carboxy-1-[3-({3-[(propylcarbamoyl)amino]benzene-1-sulfonyl}amino)phenyl]ethyl}carbamoyl)amino]phenyl}carbamoyl)-N-methylglycyl-N-methylglycyl-N-methylglycyl-N-methylglycyl-N-methylglycyl-N-methylglycyl-N-methylglycyl-N-methylglycyl-L-alpha-aspartyl-L-prolyl-L- valinate (56.0 mg, 93% purity, 71% yield). LC-MS (Method 3): Rₜ=3.76 min; MS (ESIpos): m/z=1837 [M+H]⁺
Step 2: To a solution of ((4S)-4,11-diethyl-3,14-dioxo-3,4,12,14-tetrahydro-1H-pyrano[3′,4′:6,7]indolizino[1,2-b]quinolin-4-yl N-({4-[({(1R)-2-carboxy-1-[3-({3-[(propylcarbamoyl)amino]benzene-1-sulfonyl}amino)phenyl]ethyl}carbamoyl)amino]phenyl}carbamoyl)-N-methylglycyl-N-methylglycyl-N-methylglycyl-N-methylglycyl-N-methylglycyl-N-methylglycyl-N-methylglycyl-N-methylglycyl-L-alpha-aspartyl-L-prolyl-L-valinate (56.0 mg, 93% purity, 28.2 μmol) in dioxane/water (1:1, 6 mL) was added a sodium hydroxide solution (56 μl, 1 M, 56 μmol). The solution was freeze-dried to give Compound D3 (53.0 mg, 86% purity, 86%). LC-MS: Rₜ=3.76 min; MS (ESIpos): m/z=1837 [M+H]⁺. (Method 3): ¹H-NMR (600 MHz, DMSO-d₆) δ[ppm]: 0.816 (7.52), 0.828 (16.00), 0.840 (8.35), 0.893 (5.39), 0.905 (10.18), 0.917 (6.02), 0.939 (14.07), 0.948 (13.04), 1.236 (0.70), 1.295 (6.05), 1.308 (12.11), 1.321 (5.99), 1.339 (0.96), 1.351 (3.33), 1.364 (6.19), 1.375 (5.89), 1.388 (3.06), 1.929 (2.03), 2.001 (1.46), 2.182 (2.93), 2.193 (3.39), 2.204 (2.86), 2.386 (2.43), 2.425 (1.96), 2.599 (2.36), 2.614 (2.63), 2.654 (1.43), 2.742 (6.95), 2.753 (5.75), 2.775 (8.38), 2.791 (6.09), 2.818 (4.69), 2.837 (4.59), 2.868 (9.35), 2.909 (11.58), 2.923 (11.28), 2.934 (11.48), 3.217 (4.99), 3.230 (5.26), 3.568 (7.48), 3.767 (2.00), 3.992 (3.16), 4.028 (2.96), 4.201 (3.99), 4.324 (3.09), 4.899 (1.86), 4.973 (2.03), 5.298 (1.30), 5.329 (4.76), 5.343 (4.36), 5.374 (1.13), 5.483 (10.54), 6.687 (2.03), 6.942 (2.66), 6.955 (3.16), 7.058 (2.83), 7.071 (4.46), 7.084 (2.03), 7.178 (3.19), 7.191 (3.93), 7.258 (13.54), 7.276 (4.99), 7.289 (5.59), 7.728 (2.56), 7.850 (1.80), 7.862 (2.00), 8.041 (2.43), 8.056 (1.80), 8.099 (1.86), 8.276 (3.23), 8.290 (2.96), 8.394 (0.90), 8.605 (0.43), 8.833 (0.86), 9.934 (0.86).

### Example E1: Preparation of trisodium (4S)-4,11-diethyl-3,14-dioxo-3,4,12,14-tetrahydro-1H-pyrano[3′,4′:6,7]indolizino[1,2-b]quinolin-4-yl 1-[(2S,27S)-27-[(2-carboxylatoethyl)carbamoyl]-2-(carboxylatomethyl)-42-{4-[({(1R)-2-carboxylato-1-[3-({3-[(propylcarbamoyl)amino]benzene-1-sulfonyl}amino)phenyl]ethyl}carbamoyl)amino]anilino}-4,17,21,29,42-pentaoxo-7,10,13,32,35,38-hexaoxa-3,16,22,28,41-pentaazadotetracontanan-1-oyl]-L-prolyl-L-valinate (Compound E1)

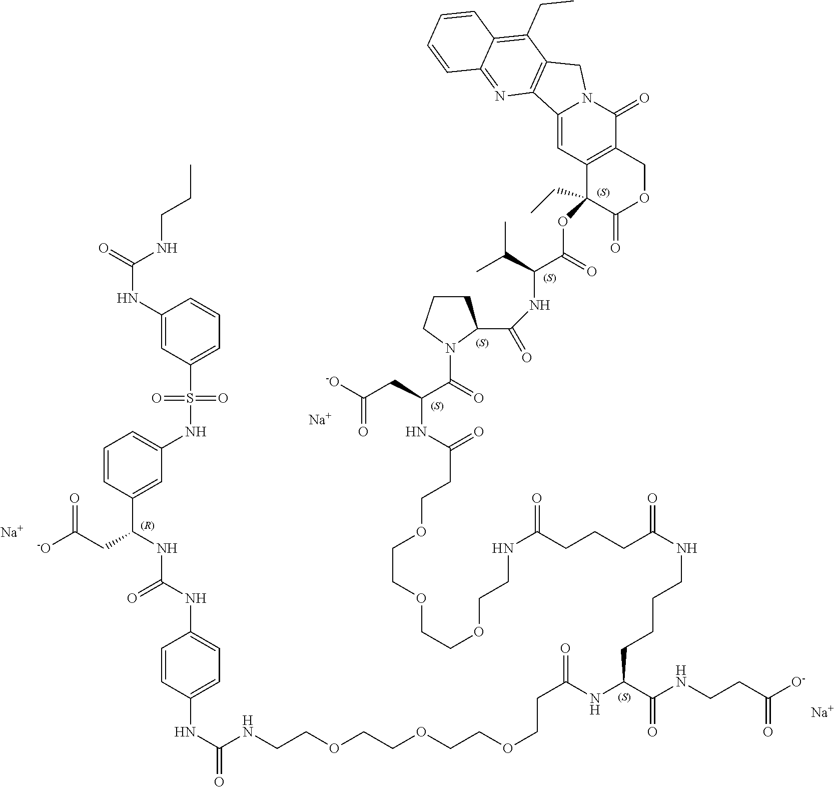

Step 1: To a solution of (4S)-4,11-diethyl-3,14-dioxo-3,4,12,14-tetrahydro-1H-pyrano[3′,4′:6,7]indolizino[1,2-b]quinolin-4-yl N-{(24S)-37-amino-24-[(2-carboxyethyl)carbamoyl]-14,18,26-trioxo-4,7,10,29,32,35-hexaoxa-13,19,25-triazaheptatriacontanan-1-oyl}-L-alpha-aspartyl-L-prolyl-L-valinate·trifluoroacetic acid (1/1) (62.0 mg, 40.7 μmol) (Intermediate 41) in DMF (13 mL) were added (3R)-3-{[(4-{[(4-nitrophenoxy) carbonyl]amino} phenyl)carbamoyl] amino}-3-{3-[({3-[(propylcarbamoyl) amino]phenyl}sulfonyl) amino]phenyl}propanoic acid (Building Block 2) (29.3 mg, 40.7 μmol) and DIEA (71 μl, 410 μmol). The reaction was stirred for 30 min at rt. The reaction was evaporated to dryness and the residue was purified by preparative HPLC to yield (4S)-4,11-diethyl-3,14-dioxo-3,4,12,14-tetrahydro-1H-pyrano[3′,4′:6,7]indolizino[1,2-b]quinolin-4-yl N-[(24S)-24-[(2-carboxyethyl)carbamoyl]-39-{4-[({(1R)-2-carboxy-1-[3-({3-[(propylcarbamoyl)amino]benzene-1-sulfonyl}amino)phenyl]ethyl}carbamoyl)amino]anilino}-14,18,26,39-tetraoxo-4,7,10,29,32,35-hexaoxa-13,19,25,38-tetraazanonatriacontanan-1-oyl]-L-alpha-aspartyl-L-prolyl-L-valinate (55.0 mg, 90% purity, 61% yield) as a yellow foam. LC-MS(Method 3): Rₜ=3.80 min; MS (ESIpos): m/z=994 [M+2H]²⁺.
Step 2: To a solution of ((4S)-4,11-diethyl-3,14-dioxo-3,4,12,14-tetrahydro-1H-pyrano[3′,4′:6,7]indolizino[1,2-b]quinolin-4-yl N-[(24S)-24-[(2-carboxyethyl)carbamoyl]-39-{4-[({(1R)-2-carboxy-1-[3-({3-[(propylcarbamoyl)amino]benzene-1-sulfonyl}amino)phenyl]ethyl}carbamoyl)amino]anilino}-14,18,26,39-tetraoxo-4,7,10,29,32,35-hexaoxa-13,19,25,38-tetraazanonatriacontanan-1-oyl]-L-alpha-aspartyl-L-prolyl-L-valinate (55.0 mg, 27.7 μmol) in dioxane/water (1:1, 10 mL) was added a sodium hydroxide solution (83 μl, 1 M, 83 μmol). The solution was freeze-dried to give Compound E¹ (53.0 mg, 97% purity, 90%) as an almost colorless foam. LC-MS (Method 1): Rₜ=0.87 min; MS (ESIpos): m/z=1984 [M+H]⁺. ¹H-NMR (600 MHz, DMSO-d₆) δ[ppm]: 0.813 (2.04), 0.826 (4.43), 0.838 (2.27), 0.873 (1.53), 0.886 (4.20), 0.898 (3.47), 0.915 (2.37), 0.925 (2.36), 1.208 (0.41), 1.233 (0.51), 1.296 (1.43), 1.309 (2.96), 1.322 (1.79), 1.343 (1.29), 1.355 (1.75), 1.367 (1.51), 1.379 (0.74), 1.663 (0.71), 1.676 (1.08), 1.688 (0.79), 2.011 (1.17), 2.023 (2.39), 2.035 (2.25), 2.048 (0.87), 2.218 (1.71), 2.227 (1.40), 2.238 (0.88), 2.268 (0.46), 2.292 (0.61), 2.333 (0.41), 2.345 (0.64), 2.360 (1.12), 2.371 (1.78), 2.381 (1.30), 2.515 (0.86), 2.518 (1.06), 2.583 (1.42), 2.609 (0.42), 2.909 (0.54), 2.919 (1.13), 2.930 (1.14), 2.942 (0.55), 2.956 (0.40), 2.967 (0.71), 2.977 (0.92), 2.987 (0.71), 2.998 (0.41), 3.152 (1.46), 3.162 (1.71), 3.171 (1.26), 3.185 (1.42), 3.195 (2.08), 3.204 (2.08), 3.220 (1.71), 3.229 (1.51), 3.354 (6.29), 3.373 (5.75), 3.414 (3.49), 3.424 (4.03), 3.437 (6.65), 3.446 (10.55), 3.461 (2.04), 3.470 (2.23), 3.478 (2.65), 3.491 (2.50), 3.496 (2.01), 3.513 (5.51), 3.535 (0.90), 3.540 (0.82), 3.551 (1.05), 3.566 (16.00), 3.576 (1.21), 3.586 (1.90), 3.597 (1.10), 3.819 (0.48), 3.833 (0.92), 3.848 (0.63), 4.130 (0.46), 4.139 (0.47), 4.841 (0.41), 4.962 (0.44), 5.139 (0.43), 5.151 (0.42), 5.325 (1.11), 5.335 (1.14), 5.462 (2.50), 6.635 (0.61), 6.648 (0.63), 6.933 (0.66), 6.946 (0.78), 7.041 (0.70), 7.054 (1.14), 7.067 (0.51), 7.167 (0.72), 7.180 (0.87), 7.217 (0.44), 7.234 (4.34), 7.277 (0.73), 7.290 (1.11), 7.304 (0.60), 7.507 (1.20), 7.554 (1.13), 7.719 (0.70), 7.731 (0.47), 7.808 (0.66), 7.848 (0.56), 7.858 (1.47), 7.872 (0.50), 7.883 (0.46), 7.892 (0.76), 7.902 (0.67), 7.912 (0.71), 7.998 (0.65), 8.010 (1.46), 8.024 (0.81), 8.176 (0.66), 8.192 (1.05), 8.205 (0.53), 8.269 (0.84), 8.284 (0.76), 8.763 (0.42), 8.866 (0.76), 11.243 (0.64).

### Example E2: Preparation of trisodium (4S)-4,11-diethyl-3,14-dioxo-3,4,12,14-tetrahydro-1H-pyrano[3′,4′:6,7]indolizino[1,2-b]quinolin-4-yl 1-[(2S,27S)-27-[(7-carboxylatoheptyl)carbamoyl]-2-(carboxylatomethyl)-42-{4-[({(1R)-2-carboxylato-1-[3-({3-[(propylcarbamoyl)amino]benzene-1-sulfonyl}amino)phenyl]ethyl}carbamoyl)amino]anilino}-4,17,21,29,42-pentaoxo-7,10,13,32,35,38-hexaoxa-3,16,22,28,41-pentaazadotetracontanan-1-oyl]-L-prolyl-L-valinate (Compound E2)

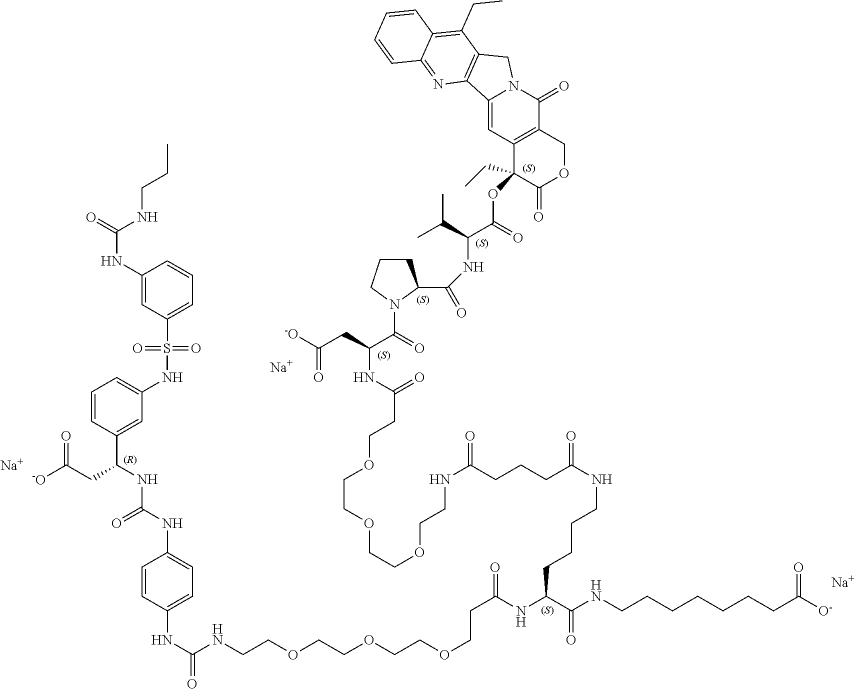

Step 1: To a solution of (4S)-4,11-diethyl-3,14-dioxo-3,4,12,14-tetrahydro-1H-pyrano[3′,4′:6,7]indolizino[1,2-b]quinolin-4-yl N-{(24S)-37-amino-24-[(7-carboxyheptyl)carbamoyl]-14,18,26-trioxo-4,7,10,29,32,35-hexaoxa-13,19,25-triazaheptatriacontanan-1-oyl}-L-alpha-aspartyl-L-prolyl-L-valinate-trifluoroacetic acid (½) (86.0 mg, 90% purity, 45.4 μmol) (Intermediate 63) in DMF (4 mL) were added (3R)-3-{[(4-{[(4-nitrophenoxy) carbonyl]amino} phenyl)carbamoyl] amino}-3-{3-[({3-[(propylcarbamoyl) amino]phenyl}sulfonyl) amino]phenyl}propanoic acid (Building Block 2) (35.9 mg, 49.9 μmol) and DIEA (40 μl, 230 μmol). The reaction was stirred for 30 min at rt. The reaction was evaporated to dryness and the residue was purified by preparative HPLC to yield (4S)-4,11-diethyl-3,14-dioxo-3,4,12,14-tetrahydro-1H-pyrano[3′,4′:6,7]indolizino[1,2-b]quinolin-4-yl N-[(24S)-24-[(7-carboxyheptyl)carbamoyl]-39-{4-[({(1R)-2-carboxy-1-[3-({3-[(propylcarbamoyl)amino]benzene-1-sulfonyl}amino)phenyl]ethyl}carbamoyl)amino]anilino}-14,18,26,39-tetraoxo-4,7,10,29,32,35-hexaoxa-13,19,25,38-tetraazanonatriacontanan-1-oyl]-L-alpha-aspartyl-L-prolyl-L-valinate (37 mg, 100% purity, 40% yield). LC-MS (Method 3): Rt=3.97 min; MS (ESIpos): m/z=1300 [M+H]⁺.
Step 2: To a solution of (4S)-4,11-diethyl-3,14-dioxo-3,4,12,14-tetrahydro-1H-pyrano[3′,4′:6,7]indolizino[1,2-b]quinolin-4-yl N-[(24S)-24-[(7-carboxyheptyl)carbamoyl]-39-{4-[({(1R)-2-carboxy-1-[3-({3-[(propylcarbamoyl)amino]benzene-1-sulfonyl}amino)phenyl]ethyl}carbamoyl)amino]anilino}-14,18,26,39-tetraoxo-4,7,10,29,32,35-hexaoxa-13,19,25,38-tetraazanonatriacontanan-1-oyl]-L-alpha-aspartyl-L-prolyl-L-valinate (35.0 mg, 17.0 μmol) in dioxane/water (1:1, 7 mL) was added a sodium hydroxide solution (51 μl, 1 M, 51 μmol). The solution was freeze-dried to give Compound E² (36 mg, 93% purity, 93% yield) as a bright yellow foam. LC-MS (Method 3): Rₜ=3.95 min; MS (ESIpos): m/z=1029 [M+H]⁺. ¹H-NMR (600 MHz, DMSO-d₆) δ[ppm]: 0.817 (0.03), 0.818 (0.04), 0.826 (0.04), 0.838 (0.04), 0.851 (0.65), 0.864 (1.38), 0.876 (0.73), 0.904 (0.92), 0.912 (1.28), 0.924 (0.54), 0.948 (0.79), 0.958 (0.80), 0.983 (0.02), 1.016 (0.02), 1.052 (0.05), 1.197 (0.05), 1.209 (0.09), 1.221 (0.14), 1.262 (1.28), 1.297 (0.07), 1.307 (0.04), 1.315 (0.03), 1.336 (0.48), 1.349 (1.00), 1.361 (0.59), 1.368 (0.33), 1.380 (0.65), 1.392 (0.89), 1.405 (0.69), 1.416 (0.30), 1.428 (0.08), 1.439 (0.03), 1.443 (0.03), 1.457 (0.09), 1.470 (0.21), 1.480 (0.31), 1.492 (0.25), 1.497 (0.18), 1.505 (0.13), 1.515 (0.12), 1.523 (0.09), 1.609 (0.05), 1.619 (0.08), 1.632 (0.10), 1.634 (0.10), 1.639 (0.09), 1.644 (0.08), 1.657 (0.06), 1.682 (0.07), 1.695 (0.23), 1.707 (0.35), 1.719 (0.26), 1.732 (0.08), 1.822 (0.05), 1.826 (0.05), 1.841 (0.08), 1.851 (0.08), 1.868 (0.06), 1.872 (0.05), 1.880 (0.03), 1.887 (0.03), 1.942 (0.25), 1.954 (0.47), 1.966 (0.30), 1.985 (0.12), 1.990 (0.11), 1.998 (0.13), 2.008 (0.10), 2.017 (0.13), 2.019 (0.13), 2.026 (0.12), 2.042 (0.31), 2.056 (0.65), 2.069 (0.64), 2.082 (0.31), 2.091 (0.11), 2.093 (0.11), 2.099 (0.10), 2.110 (0.12), 2.117 (0.07), 2.124 (0.09), 2.134 (0.14), 2.142 (0.15), 2.159 (0.15), 2.167 (0.12), 2.190 (0.03), 2.224 (0.03), 2.227 (0.03), 2.233 (0.03), 2.234 (0.03), 2.249 (0.07), 2.261 (0.11), 2.273 (0.22), 2.285 (0.35), 2.296 (0.27), 2.308 (0.21), 2.319 (0.21), 2.329 (0.11), 2.340 (0.03), 2.345 (0.03), 2.354 (0.06), 2.359 (0.05), 2.365 (0.11), 2.377 (0.19), 2.388 (0.35), 2.399 (0.23), 2.413 (0.17), 2.422 (0.30), 2.430 (0.24), 2.443 (0.19), 2.455 (0.19), 2.461 (0.20), 2.471 (0.09), 2.480 (0.04), 2.482 (0.04), 2.487 (0.04), 2.500 (0.08), 2.511 (0.12), 2.559 (0.36), 2.577 (16.00), 2.596 (0.11), 2.616 (0.03), 2.620 (0.02), 2.650 (0.15), 2.683 (0.11), 2.690 (0.10), 2.699 (0.12), 2.707 (0.11), 2.717 (0.03), 2.725 (0.10), 2.944 (0.15), 2.954 (0.35), 2.966 (0.36), 2.977 (0.17), 2.988 (0.13), 3.008 (0.27), 3.014 (0.28), 3.025 (0.21), 3.036 (0.20), 3.046 (0.21), 3.056 (0.20), 3.067 (0.16), 3.079 (0.10), 3.089 (0.06), 3.100 (0.03), 3.172 (0.14), 3.182 (0.41), 3.191 (0.48), 3.201 (0.35), 3.210 (0.40), 3.219 (0.46), 3.229 (0.27), 3.242 (0.21), 3.255 (0.22), 3.265 (0.22), 3.278 (0.21), 3.287 (0.19), 3.300 (0.20), 3.424 (0.50), 3.435 (0.64), 3.444 (0.83), 3.454 (0.57), 3.466 (2.31), 3.471 (3.38), 3.491 (0.23), 3.518 (0.85), 3.525 (0.89), 3.544 (1.75), 3.564 (0.25), 3.570 (0.22), 3.580 (0.30), 3.592 (0.18), 3.604 (1.87), 3.622 (0.51), 3.634 (0.43), 3.646 (0.23), 3.650 (0.19), 3.662 (0.10), 3.670 (0.05), 3.673 (0.04), 3.677 (0.03), 3.684 (0.04), 3.692 (0.03), 3.695 (0.03), 3.702 (0.03), 3.704 (0.03), 3.710 (0.02), 3.714 (0.03), 3.718 (0.03), 3.726 (0.03), 3.729 (0.03), 3.734 (0.03), 3.741 (0.03), 3.744 (0.03), 3.747 (0.03), 3.763 (0.15), 3.777 (0.24), 3.791 (0.14), 3.808 (0.03), 3.848 (0.03), 3.850 (0.02), 3.861 (0.04), 3.869 (0.07), 3.875 (0.06), 3.886 (0.14), 3.899 (0.14), 3.915 (0.06), 4.028 (0.03), 4.048 (0.10), 4.064 (0.15), 4.071 (0.07), 4.079 (0.08), 4.087 (0.04), 4.099 (0.02), 4.170 (0.09), 4.175 (0.06), 4.184 (0.17), 4.193 (0.16), 4.206 (0.08), 4.366 (0.07), 4.817 (0.07), 4.825 (0.09), 4.830 (0.12), 4.838 (0.12), 4.854 (0.07), 4.871 (0.02), 4.874 (0.03), 4.976 (0.13), 5.282 (0.17), 5.296 (0.18), 5.332 (0.07), 5.363 (0.41), 5.371 (0.43), 5.403 (0.07), 5.489 (0.88), 5.514 (0.06), 5.520 (0.03), 6.594 (0.04), 6.598 (0.04), 6.609 (0.06), 6.614 (0.06), 6.621 (0.05), 6.641 (0.02), 6.862 (0.02), 6.867 (0.02), 6.872 (0.02), 6.881 (0.02), 6.892 (0.02), 6.900 (0.02), 7.002 (0.04), 7.014 (0.06), 7.018 (0.06), 7.022 (0.06), 7.025 (0.06), 7.028 (0.05), 7.036 (0.05), 7.039 (0.04), 7.171 (0.12), 7.183 (0.15), 7.247 (0.35), 7.262 (0.68), 7.268 (0.31), 7.285 (0.74), 7.300 (0.33), 7.330 (0.03), 7.381 (0.03), 7.400 (0.03), 7.405 (0.02), 7.412 (0.03), 7.415 (0.03), 7.419 (0.04), 7.432 (0.07), 7.436 (0.08), 7.442 (0.09), 7.443 (0.09), 7.452 (0.11), 7.453 (0.11), 7.460 (0.10), 7.486 (0.05), 7.490 (0.04), 7.493 (0.03), 7.501 (0.03), 7.507 (0.02), 7.532 (0.02), 7.544 (0.04), 7.557 (0.26), 7.712 (0.60), 7.742 (0.14), 7.754 (0.24), 7.767 (0.17), 7.825 (0.11), 7.835 (0.22), 7.844 (0.16), 7.858 (0.14), 7.860 (0.13), 7.873 (0.06), 7.875 (0.06), 7.883 (0.18), 7.896 (0.27), 7.909 (0.16), 7.963 (0.11), 7.973 (0.22), 7.981 (0.14), 7.997 (0.02), 8.036 (0.41), 8.049 (0.39), 8.068 (0.04), 8.073 (0.04), 8.078 (0.03), 8.081 (0.03), 8.112 (0.02), 8.141 (0.24), 8.154 (0.28), 8.168 (0.10), 8.169 (0.10), 8.183 (0.23), 8.197 (0.22), 8.220 (0.04), 8.224 (0.03), 8.228 (0.03), 8.232 (0.03), 8.252 (0.02), 8.307 (0.29), 8.320 (0.27), 8.333 (0.03), 8.563 (0.21), 8.776 (0.04), 8.784 (0.05), 8.787 (0.05), 8.795 (0.05), 8.800 (0.05), 8.809 (0.05), 8.823 (0.03), 8.832 (0.03), 8.915 (0.02), 8.923 (0.04), 8.926 (0.04), 8.933 (0.06), 8.941 (0.07), 8.946 (0.07), 8.950 (0.06), 8.963 (0.04), 8.965 (0.04), 9.068 (0.14), 9.081 (0.15), 9.616 (0.05), 9.623 (0.06), 9.627 (0.06), 9.634 (0.06), 9.637 (0.05), 9.643 (0.04), 9.646 (0.03), 11.204 (0.02), 11.211 (0.02), 11.219 (0.02), 11.226 (0.03), 11.233 (0.03), 11.239 (0.03), 11.247 (0.03), 11.251 (0.03), 11.262 (0.03), 11.266 (0.03), 11.270 (0.03), 11.274 (0.03), 11.281 (0.03), 11.283 (0.02), 11.289 (0.02), 11.300 (0.02), 11.328 (0.02).

### Example E3: Preparation of trisodium N-{(2S,18S)-1-carboxylato-18-[(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-2-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-4,17,24,28-tetraoxo-7,10,13-trioxa-3,16,23-triazaoctacosan-28-yl}-N-methylglycyl-N-methylglycyl-N-(carboxylatomethyl)-N,N2-dimethylglycinamide (Compound E3)

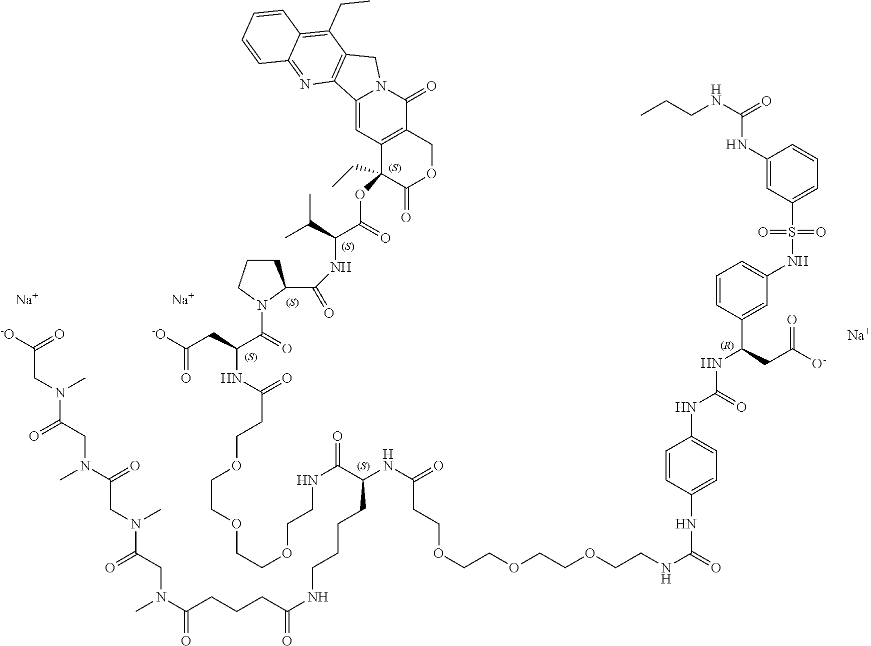

Step 1: To a solution of (23S,39S)-23-(3-{2-[2-(2-aminoethoxy)ethoxy]ethoxy}propanamido)-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid·trifluoroacetic acid (1/1) (46.0 mg, 87% purity, 23.1 μmol) (Intermediate 57) in DMF (5 mL) were added (3R)-3-{[(4-{[(4-nitrophenoxy) carbonyl]amino} phenyl)carbamoyl] amino}-3-{3-[({3-[(propylcarbamoyl) amino]phenyl}sulfonyl) amino]phenyl}propanoic acid (Building block 2) (16.6 mg, 23.1 μmol) and DIEA (40 μl, 230 μmol). The reaction was stirred for 30 min at rt. The reaction was evaporated to dryness and the residue was purified by preparative HPLC to yield (23 S,39S)-23-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid (20 mg, 94% purity, 37% yield) as a yellow foam. LC-MS (Method 3): Rₜ=3.83 min; MS (ESIpos): m/z=2200 [M+H]⁺.
Step 2: To a solution of (23S,39S)-23-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-39-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12-tetramethyl-4,7,10,13,17,24,37-heptaoxo-28,31,34-trioxa-3,6,9,12,18,25,38-heptaazahentetracontane-1,41-dioic acid (20.0 mg, 9.08 μmol) in dioxane/water (1:1, 4 mL) was added a sodium hydroxide solution (27 μl, 1 M, 27 μmol). The solution was freeze-dried to give Compound E3 (21 mg, 96% purity, 97% yield) as a colorless foam. LC-MS (Method 3): Rₜ=3.83 min; MS (ESIpos): m/z=2197 [M+H]⁺. ¹H-NMR (600 MHz, DMSO-d₆) δ[ppm]: 0.819 (3.33), 0.831 (7.08), 0.843 (3.68), 0.893 (2.51), 0.905 (4.58), 0.917 (2.77), 0.928 (4.07), 0.935 (5.64), 0.945 (4.37), 1.012 (0.56), 1.202 (0.55), 1.234 (0.69), 1.296 (2.55), 1.309 (5.01), 1.321 (2.89), 1.344 (1.30), 1.356 (2.04), 1.368 (2.77), 1.380 (2.46), 1.392 (1.25), 1.455 (0.49), 1.469 (0.53), 1.586 (0.63), 1.625 (0.46), 1.638 (0.57), 1.650 (0.46), 1.696 (0.65), 1.920 (1.26), 1.931 (1.05), 2.000 (0.95), 2.026 (1.37), 2.066 (0.83), 2.079 (1.23), 2.091 (0.78), 2.108 (0.57), 2.122 (0.65), 2.196 (1.16), 2.207 (1.25), 2.216 (1.20), 2.261 (0.92), 2.274 (0.97), 2.288 (0.90), 2.300 (0.74), 2.313 (0.83), 2.324 (1.25), 2.339 (1.16), 2.351 (1.85), 2.361 (1.51), 2.377 (1.11), 2.387 (1.33), 2.522 (0.97), 2.587 (0.83), 2.615 (0.66), 2.676 (0.72), 2.698 (1.02), 2.712 (2.50), 2.719 (1.36), 2.725 (1.80), 2.735 (1.83), 2.747 (2.01), 2.778 (1.97), 2.785 (2.29), 2.794 (1.98), 2.809 (2.82), 2.820 (1.68), 2.840 (1.79), 2.849 (1.53), 2.854 (1.27), 2.884 (2.10), 2.903 (1.98), 2.915 (1.17), 2.939 (2.77), 2.948 (3.29), 2.962 (2.13), 3.133 (0.92), 3.144 (1.13), 3.152 (1.27), 3.163 (1.36), 3.202 (3.20), 3.212 (3.14), 3.226 (2.30), 3.358 (3.97), 3.367 (5.24), 3.377 (4.05), 3.395 (3.72), 3.450 (10.73), 3.459 (16.00), 3.472 (6.04), 3.477 (5.80), 3.492 (7.14), 3.510 (8.81), 3.528 (3.05), 3.532 (2.90), 3.548 (3.46), 3.552 (3.72), 3.568 (11.15), 3.577 (3.41), 3.586 (4.77), 3.591 (4.75), 3.599 (5.53), 3.604 (4.32), 3.612 (3.34), 3.628 (1.95), 3.632 (1.91), 3.694 (1.32), 3.809 (1.79), 3.832 (0.90), 3.840 (0.60), 3.908 (0.50), 3.924 (0.56), 3.959 (0.85), 3.974 (1.57), 3.982 (1.67), 3.987 (1.73), 3.994 (2.05), 4.001 (1.41), 4.007 (1.16), 4.015 (0.90), 4.032 (0.57), 4.060 (0.66), 4.098 (0.55), 4.108 (0.42), 4.116 (0.47), 4.163 (1.35), 4.178 (1.43), 4.262 (0.47), 4.284 (0.64), 4.330 (1.00), 4.836 (1.48), 4.840 (2.68), 4.844 (1.46), 4.883 (0.69), 4.894 (0.74), 4.909 (0.90), 4.978 (1.27), 5.296 (0.42), 5.327 (1.91), 5.337 (1.95), 5.368 (0.40), 5.482 (4.21), 6.683 (1.05), 6.696 (1.04), 6.950 (0.89), 6.962 (1.02), 7.059 (1.01), 7.071 (1.59), 7.084 (0.74), 7.183 (1.31), 7.196 (1.78), 7.206 (1.22), 7.221 (2.45), 7.234 (2.77), 7.249 (1.09), 7.275 (1.08), 7.288 (1.66), 7.301 (0.87), 7.377 (1.00), 7.389 (0.74), 7.613 (0.89), 7.716 (0.92), 7.728 (2.02), 7.740 (1.45), 7.851 (0.79), 7.864 (1.21), 7.876 (0.74), 7.954 (0.46), 8.011 (0.55), 8.035 (1.72), 8.049 (1.79), 8.274 (1.56), 8.288 (1.48), 8.313 (0.60), 8.375 (0.68).

### Example E4: Preparation of trisodium N-{(2S,18S)-1-carboxylato-18-[(14-{4-[({(1R)-2-carboxylato-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-2-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-4,17,24,28-tetraoxo-7,10,13-trioxa-3,16,23-triazaoctacosan-28-yl}-N-methylglycyl-N-methylglycyl-N-methylglycyl-N-methylglycyl-N-methylglycyl-N-methylglycyl-N-(carboxylatomethyl)-N,N2-dimethylglycinamide (Compound E4)

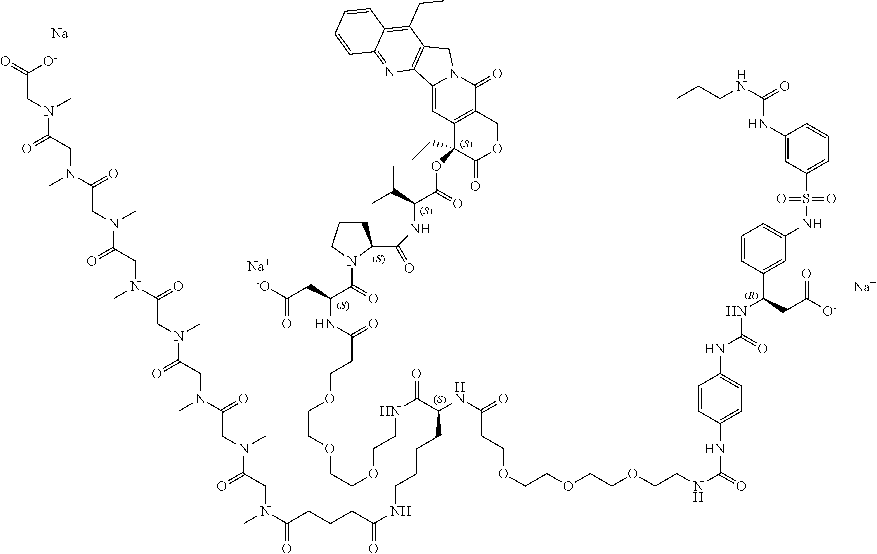

Step 1: To a solution of (35S,51S)-35-(3-{2-[2-(2-aminoethoxy)ethoxy]ethoxy}propanamido)-51-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12,15,18,21,24-octamethyl-4,7,10,13,16,19,22,25,29,36,49-undecaoxo-40,43,46-trioxa-3,6,9,12,15,18,21,24,30,37,50-undecaazatripentacontane-1,53-dioic acid-trifluoroacetic acid (1/1) (27.0 mg, 13.4 μmol) (Intermediate 60) in DMF (5.8 mL) was added (3R)-3-{[(4-{[(4-nitrophenoxy) carbonyl]amino} phenyl)carbamoyl] amino}-3-{3-[({3-[(propylcarbamoyl) amino]phenyl}sulfonyl) amino]phenyl}propanoic acid (Building Block 2) (9.62 mg, 13.4 μmol) and DIEA (71 μl, 410 μmol). The reaction was stirred for 30 min at rt. The reaction was evaporated to dryness and the residue was purified by preparative HPLC to yield (35S,51S)-35-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-51-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12,15,18,21,24-octamethyl-4,7,10,13,16,19,22,25,29,36,49-undecaoxo-40,43,46-trioxa-3,6,9,12,15,18,21,24,30,37,50-undecaazatripentacontane-1,53-dioic acid (10 mg, 100% purity, 30% yield) as a yellow foam. LC-MS (Method 3): Rₜ=3.75 min; MS (ESIpos): m/z=2485 [M+H]⁺.
Step 2: To a solution of (35S,51S)-35-[(14-{4-[({(1R)-2-carboxy-1-[3-({3-[(propylcarbamoyl)amino]benzene-1-sulfonyl}amino)phenyl]ethyl}carbamoyl)amino]anilino}-14-oxo-4,7,10-trioxa-13-azatetradecanan-1-oyl)amino]-51-[(2S)-2-{[(2S)-1-{[(4S)-4,11-diethyl-3,14-dioxo-3,4,12,14-tetrahydro-1H-pyrano[3′,4′:6,7]indolizino[1,2-b]quinolin-4-yl]oxy}-3-methyl-1-oxobutan-2-yl]carbamoyl}pyrrolidine-1-carbonyl]-3,6,9,12,15,18,21,24-octamethyl-4,7,10,13,16,19,22,25,29,36,49-undecaoxo-40,43,46-trioxa-3,6,9,12,15,18,21,24,30,37,50-undecaazatripentacontane-1,53-dioic acid (10.0 mg, 4.02 μmol) in dioxane/water (1:1, 3 mL) was added a sodium hydroxide solution (120 μl, 0.1 M, 12 μmol). The solution was freeze-dried to give Compound E4 (9.7 mg, 94% purity, 89% yield) as an almost colorless foam. LC-MS (Method 3): Rₜ=3.74 min; MS (ESIpos): m/z=2482 [M+H]⁺. ¹H-NMR (600 MHz, DMSO-d₆) δ[ppm]: 0.816 (2.77), 0.828 (5.74), 0.840 (3.14), 0.883 (2.31), 0.896 (4.56), 0.907 (5.06), 0.923 (4.25), 0.934 (3.44), 1.009 (0.48), 1.236 (1.72), 1.297 (2.33), 1.310 (4.59), 1.322 (2.94), 1.347 (2.08), 1.359 (2.66), 1.371 (2.24), 1.383 (1.17), 1.464 (0.62), 1.580 (0.63), 1.647 (0.60), 1.690 (0.80), 1.922 (0.80), 2.012 (1.38), 2.079 (1.63), 2.220 (1.46), 2.310 (2.48), 2.352 (1.44), 2.364 (1.53), 2.386 (1.70), 2.425 (0.57), 2.578 (0.90), 2.614 (0.78), 2.654 (0.41), 2.689 (1.35), 2.744 (3.73), 2.774 (4.44), 2.799 (2.99), 2.825 (2.68), 2.869 (2.53), 2.924 (4.47), 3.181 (3.24), 3.190 (3.61), 3.224 (2.90), 3.443 (10.40), 3.452 (14.60), 3.478 (6.09), 3.488 (7.23), 3.510 (7.84), 3.568 (16.00), 3.740 (0.50), 3.827 (0.77), 3.893 (1.34), 4.000 (1.19), 4.053 (1.26), 4.090 (1.03), 4.185 (2.03), 4.272 (0.84), 4.317 (1.11), 4.854 (0.71), 4.968 (0.84), 5.057 (0.55), 5.329 (1.89), 5.339 (1.99), 5.471 (3.85), 6.647 (1.08), 6.659 (1.03), 6.955 (1.07), 7.045 (0.85), 7.058 (1.34), 7.072 (0.61), 7.172 (1.20), 7.185 (1.47), 7.225 (2.64), 7.234 (2.71), 7.278 (0.89), 7.291 (1.42), 7.304 (0.76), 7.401 (0.45), 7.471 (0.77), 7.535 (1.42), 7.713 (0.76), 7.725 (1.30), 7.738 (0.95), 7.825 (1.18), 7.851 (0.89), 7.863 (1.22), 7.876 (0.78), 7.990 (0.55), 8.024 (1.55), 8.038 (1.26), 8.079 (0.45), 8.160 (0.87), 8.245 (0.62), 8.274 (1.44), 8.289 (1.28), 8.693 (0.48), 8.902 (0.50), 11.172 (0.52).

### Biological Evaluation

### Example F1: Cytotoxicity In Vitro in the Presence and Absence of Elastase

Cultivation of cells was performed according to standard procedures with the media recommended by the provider. The cells, in a total volume of 100 μL, were seeded in a 96-well plate with a white bottom (#3610). After a 24 h incubation period at 37° C. and 5% CO₂, the medium was exchanged by adding 90 μL fresh medium. The treatment started by adding the test compound in 10 μL of culture medium to the cells in triplicates. Concentrations ranging from 10⁻⁵ M to 10⁻¹³ M were chosen. Two identically treated sets of samples were prepared. One set was treated with the test compound alone, whereas to the second set the compound and 10 nM elastase was added followed by a 72 h incubation period at 37° C. and 5% CO₂. The proliferation was detected using the MTT assay (ATCC). At the end of the incubation period the MTT reagent was added to all samples for 4h. Lysis of the cells followed by addition of the detergent was done overnight. The formed dye was detected at 570 nm. The proliferation of untreated but otherwise identically handled cells was define as the 10000 value. The dose response curves allowed the determination of the respective IC50 values, which are summarized in table 1.

TABLE 1
Determination of the IC₅₀ Values

| | 786-O | | HT29 | | NCI H292 | | SUM149 | |
|---|---|---|---|---|---|---|---|---|
| | IC₅₀ | | IC₅₀ | | IC₅₀ | | IC₅₀ | |
| Compound | −elastase | +elastase | −elastase | +elastase | −elastase | +elastase | −elastase | +elastase |
| A1 | D | B | DD | A | D | B | D | A |
| A2 | D | A | DD | A | D | A | D | A |
| A3 | D | A | DD | A | DD | A | C | A |
| A4 | D | A | D | A | D | A | C | A |
| A5 | D | A | D | A | C | AA | C | AA |
| A6 | D | B | D | B | C | B | C | A |
| A7 | D | B | D | B | C | B | C | B |
| A8 | D | B | D | B | C | B | C | B |
| B1 | D | B | DD | B | D | B | D | A |
| B2 | C | AA | D | AA | C | AA | B | AA |
| B3 | C | A | C | A | C | A | C | AA |
| B4 | D | A | D | A | D | A | C | AA |
| B4b | D | B | D | B | D | B | D | A |
| B5 | C | A | D | A | C | A | B | AA |
| C1 | D | A | D | A | D | A | D | A |
| C2 | D | B | C | B | D | B | B | A |
| C3 | D | B | D | B | D | B | C | A |
| C4 | D | A | DD | A | C | A | C | A |
| C6 | D | B | D | B | D | B | D | A |
| C7 | D | B | DD | B | C | A | C | AA |
| C8 | D | B | DD | B | C | B | C | B |
| C9 | D | C | D | B | D | B | D | B |
| D1 | D | C | D | C | D | C | D | B |
| D2 | D | B | D | B | D | B | C | B |

AA = <0.01 nM;
A = 0.01-0.1 nM;
B = 0.1-1 nM;
C = 1.1-10 nM;
D = 10-100 nM;
DD = >100 nM

The Cytotoxicity assay (Version 2) was performed as above described. Some changes were applied as the assays were performed in an automated fashion and the IC50 values determination was changed. Here the change in metabolic activity of the cells, in percent, was calculated by normalization of the measured values to the absorbance values of medium containing wells without cells (=−100% ) and the absorbance of the DMSO-treated cells (=0% ). AC₅₀ values (activity concentration at -50% o) were determined by means of a 4 parameter Hill-fit using GeneData Screener software. Values are summarized in Table 1b.

TABLE 2b
Determination of the AC₅₀ Values

| | 786-O | | HT29 | | NCI H292 | | SUM149 | |
|---|---|---|---|---|---|---|---|---|
| | AC₅₀ | | AC₅₀ | | AC₅₀ | | AC₅₀ | |
| Compound | −elastase | +elastase | −elastase | +elastase | −elastase | +elastase | −elastase | +elastase |
| A3 | C | A | C | B | C | A | C | A |
| A4 | C | A | C | B | C | A | C | A |
| A9 | C | A | D | B | C | A | C | A |
| A11 | B | A | C | B | A | A | A | A |
| A12 | A | A | C | B | B | A | A | A |
| B6 | C | A | D | B | C | A | C | A |
| C5 | C | A | D | B | C | A | C | A |
| C10 | B | A | C | B | B | A | B | A |
| D3 | C | A | C | B | C | A | C | A |
| E1 | C | A | C | B | C | A | C | A |
| E2 | C | A | C | B | C | A | C | A |
| E3 | C | A | C | B | C | A | C | A |
| E4 | C | A | C | B | C | A | C | A |

A ≤ 10 nM
B = 11-100 nM
C = 0.11-1 μM
D > 1 μM
n.t. = not tested

### Example F3: αᵥβ₃ Binding Assay

Binding affinity of the respective αᵥβ3 inhibitor is tested in an ELISA-like manner. 96-well plates are coated with vitronectin (1p g/ml) or fibronectin 0.5 μg/ml) and afterwards uncoated surface space is blocked with BSA. After a 2 h pre-incubation of a serial dilution of the antagonist of interest (starting at 20 μM in a 1:5 dilution) with the extracellular domain of the integrin (2 μg/ml, αvβ3 or a5β1) the solution is added to the coated plates. After an 1 h incubation at RT and several washing steps, an integrin specific antibody is added for 1 h at RT. After three washing steps the secondary peroxidase-labeled antibody is added to the plate. After an 1 h incubation at RT the plate is developed by quick addition of SeramunBlau (50 μL per well, Seramun Diagnostic GmbH, Heidesee, Germany) and incubated for 5 min at RT in the dark. The reaction is stopped with 0.3M H2SO4 (50 μL/well), and the absorbance is measured at 450 nm with a plate reader.

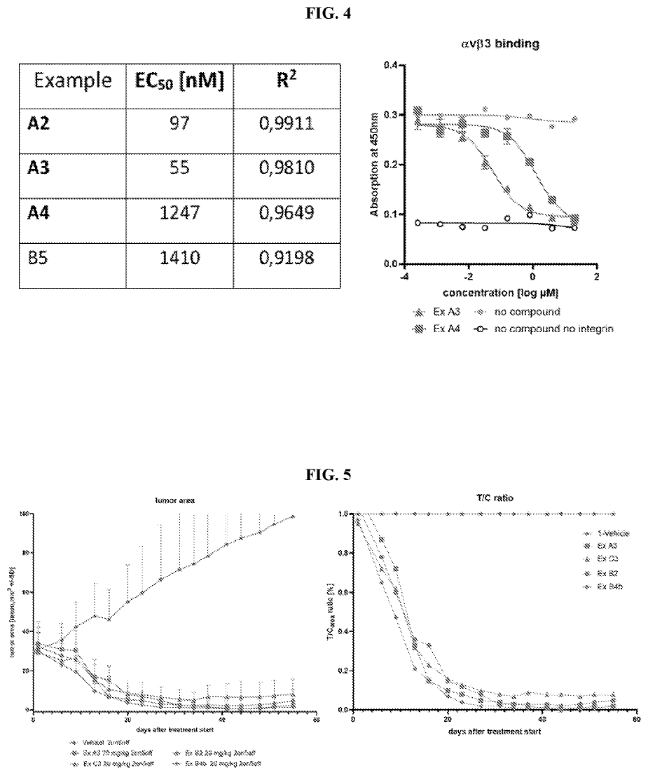

IC₅₀ of each compound was tested in duplicate, and the resulting inhibition curves are analyzed. As a reference standard (Ref-1), cilengitide is used. The αvβ3 binding is used as 100% value. The avb3 binding of selected examples and EC50 values can be seen in FIG. 4.

### Example F4: Compound Stability

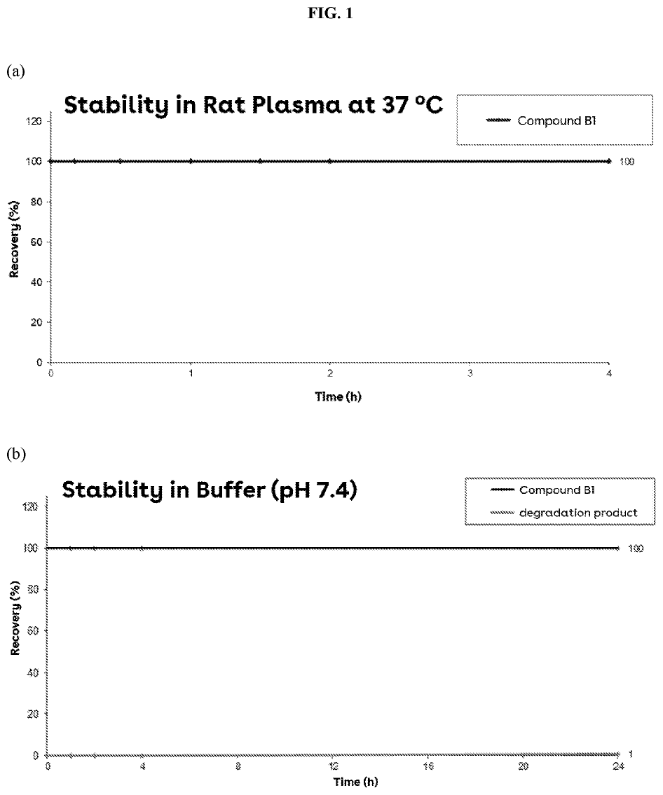

Compound B1, Compound C2, and Compound C3 were evaluated for stability in plasma and/or buffer. Compound B1 was stable in rat plasma at 37° C. (FIG. 1 panel (a)) and in buffer at pH 7.4 (FIG. 1 panel (b)) for the duration of the 4-hour assay. Compound C2 and Compound C3 show slow ester cleavage and release of the aspartate moiety in rat plasma within the duration of the 4-hour assay as detected by LC-MS, (FIG. 2 and FIG. 3 respectively). No release of free payload is detected in buffer at pH 4 within 24 hours.

Method for measurement of stability in rat and human plasma: 1 mg of the test compound is dissolved in 0.5 ml acetonitrile/dimethylsulfoxide 1:1. For complete dissolution the HPLC vial is shaken and sonicated. While vortexing 20 μl of this solution are added to 1 ml 37° C. warm plasma. After 0.17, 0.5, 1, 1.5, 2 and 4 hours the enzymatic reaction is stopped by adding 100 μl of the compound plasma solution to a vial containing 300 μl acetonitrile/buffer pH3 (80:20) at RT. The mixture is centrifuged at 5000 rpm for 10 minutes. The supernatant is analyzed by HPLC to determine the amount of the test compound and up to two byproducts. To values result from a processed sample immediately taken after vortexing with plasma at RT. The peak areas (in percentage) are used for quantification. All data is given as percent area of the initial compound at to.

Method for measurement of stability in buffer: 0.15 mg of the test compound are solved in 0.1 ml dimethylsulfoxide and 0.4 ml acetonitrile. For complete dissolution the HPLC vial with the sample solution is shaken and sonicated. Then 1.0 ml of the respective buffer solution is added, and the sample is vortexed. The sample solution is analysed by HPLC to determine the amount of the test compound and up to two byproducts at a particular time over a period of 24 h at 37° C. to values result from a sample immediately taken after vortexing with buffer at RT. The peak areas (in percentage) are used for quantification.

### Example F5: In Vitro Tests for Determining Cellular Permeability

Caco-2 assay: The cell permeability of a substance can be investigated by means of in vitro testing in a flux assay using Caco-2 cells [M. D. Troutman and D. R. Thakker, Pharm. Res. 20 (8), 1210-1224 (2003)]. For this purpose, the cells are cultured for 15-16 days on 24-well filter plates. For the determination of permeation, the respective test substance is applied in a HEPES buffer to the cells either apically (A) or basally (B) and incubated for 2 hours. After 0 hours and after 2 hours, samples are taken from the cis and trans compartments. The samples are separated by HPLC (Agilent 1200, Boblingen, Germany) using reverse phase columns. The HPLC system is coupled via a Turbo Ion Spray Interface to a Triple Quadropol mass spectrometer API 4000 (AB SCIEX Deutschland GmbH, Darmstadt, Germany). The permeability is evaluated on the basis of a Papp value, which is calculated using the formula published by Schwab et al. [D. Schwab et al., J. Med. Chem. 46, 1716-1725 (2003)]. A substance is classified as actively transported when the ratio of Papp (B-A) to Papp (A-B) (efflux ratio) is >2 or <0.5.

P-glycoprotein (P-gp) assay: Many tumor cells express transporter proteins for drugs, and this frequently accompanies the development of resistance towards cytostatics. Substances which are not substrates of such transporter proteins, such as P-glycoprotein (P-gp) or BCRP, for example, could therefore exhibit an improved activity profile. The substrate properties of a substance for P-gp (ABCB1) were determined by means of a flux assay using LLC-PKl cells which overexpress P-gp (L-MDR1 cells) [A. H. Schinkel et al., J. Clin. Invest. 96, 1698-1705 (1995)]. For this purpose, the LLC-PKl cells or L-MDR1 cells are cultured on 96-well filter plates for 3-4 days. For determination of the permeation, the respective test substance, alone or in the presence of an inhibitor (such as ivermectin or verapamil, for example), is applied in a HEPES buffer to the cells either apically (A) or basally (B) and incubated for 2 hours. After 0 hours and after 2 hours, samples are taken from the cis and trans compartments. The samples are separated by HPLC using reverse phase columns. The HPLC system is coupled via a Turbo Ion Spray Interface to a Triple Quadropol mass spectrometer API 3000 (Applied Biosystems Applera, Darmstadt, Germany). The permeability is evaluated on the basis of a Papp value, which is calculated using the formula published by Schwab et al. [D. Schwab et al., J. Med. Chem. 46, 1716-1725 (2003)]. A substance is classified as P-gp substrate when the efflux ratio of Papp (B-A) to Papp (A-B) is >2.

### Example F6: Pharmacokinetics

Male rats (n=3) were given an intravenous 0.5 mg/kg bolus dose of test article dissolved in plasma with 1% DMSO and 1% ethanol (Ex A3 only). Serial blood samples were collected up to 24 hours post-dose. Plasma was harvested from blood samples and stored frozen until sample analysis. Test article plasma concentrations were analyzed by LC/MS/MS. Pharmacokinetics parameters were calculated using non-compartmental analysis and are presented in Table 2.

TABLE 2
Pharmacokinetics in rat after intravenous
administration of test articles.

| | IV Dose | CL | Vss | AUC | t_{1/2} |
|---|---|---|---|---|---|
| Compound | (mg/kg) | (mL/min/kg) | (L/kg) | (mg · h/L) | (h) |
| B4b | 0.5 | B | B | C | B |
| A3 | 0.5 | A | A | A | A |
| E1 | 0.5 | B | B | B | B |
| E2 | 0.5 | B | A | A | B |
| E3 | 0.5 | B | B | B | B |
| | | wherein | wherein | wherein | wherein |
| | | A: 0.05-0.5 | A: 0.01-0.1 | A: 50-150 | A: 10-15 |
| | | B: 0.5-5 | B: 0.1-0-2 | B: 10-50 | B: 5-10 |
| | | C: >5 | C: 0.2-0.6 | C: 1-10 | C: 1-5 |

### Example F7: In Vivo Xenograft Mouse Studies

The anti-tumor activities of test compounds are examined in murine xenotransplantation models of human cancer. For this purpose, immunocompromised mice are implanted subcutaneously with tumor cells or tumor fragments. At a mean tumor size of 20-40 mm², animals are randomized split into treatment and control groups (n=10 animals/group) and treatment starts with vehicle-only, or test compound (formulation: phosphate buffered saline (“PBS”); application route: intravenously into the tail vein (“i.v.”)). Intravenous treatments are performed on two consecutive days once daily followed by five days of drug holiday without any treatment. The tumor size and the body weight are determined twice weekly. The tumor area is detected by means of an electronic caliper [length (mm)×width (mm)]. The experimental groups are ended when the groups reached the pre-determined ethical endpoint based on German and European animal welfare regulations. In vivo anti-tumor efficacy is presented as T/C ratio of mean tumor area measured for treatment and control group on the last day at which the vehicle control remains in study (Treatment/Control; mean tumor area of treatment group/mean tumor area of control group. A compound having a T/C below 0.5 is defined as active (i.e., effective). Statistical analysis is assessed using SigmaStat software. A one-way analysis of variance is performed and differences to the control are compared by a pair-wise comparison procedure (Dunn's method).

We claim:

1. A compound having the structure of Formula (I): 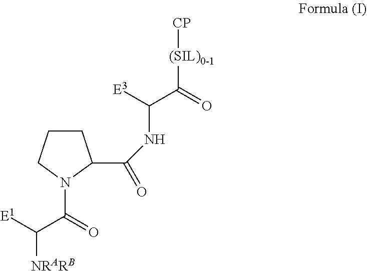
or a pharmaceutically acceptable salt thereof, or a stereoisomer or mixture of stereoisomers thereof, wherein:
CP is a cytotoxic or cytostatic group;
SIL is a self-immolative linker;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
R^{A} is hydrogen or C₁₋₆ alkyl;
R^{B} is -L¹-A¹(L²-(IN))(L³-(IN)), -L⁴-IN, L⁵-IN, L⁷-IN, or -L¹-A¹(L²-(IN))(L³-(MOD));
wherein:
each of L¹, L², L³, and L⁵ is, independently, a bivalent linker;
L⁴ is a bivalent polyamine or polyamide linker;
L⁷ is a substituted or unsubstituted C₁₋₆₀ alkyl, or substituted or unsubstituted heteroalkyl comprising 4-12 heteroalkyl units, wherein each Z group is independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, and —NHS(O)₂NHC(O)—;
A¹ is a trivalent linker;
MOD is a physicochemical or pharmacokinetic modulator;
with the proviso that when R^{B} is L⁵, ^{E} is E²; or E³ is —CH₃;
wherein E² is —CH₂C(O)OR¹ or —CH₂CH₂C(O)OR¹;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and
IN is, in each instance, independently, an integrin binder.

2. The compound of claim 1, or a pharmaceutically acceptable salt thereof, or a stereoisomer or mixture of stereoisomers thereof, having the structure of Formula (I-A): 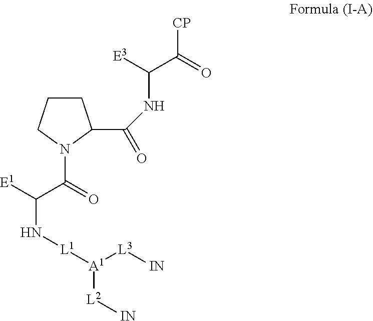
wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each of L¹, L², and L³ is independently a bivalent linker;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a bivalent linker; and
IN is in each instance, independently, a monovalent radical of an integrin binder.

3. The compound of claim 1, or a pharmaceutically acceptable salt thereof, or a stereoisomer or mixture of stereoisomers thereof, having the structure of Formula (I-B): 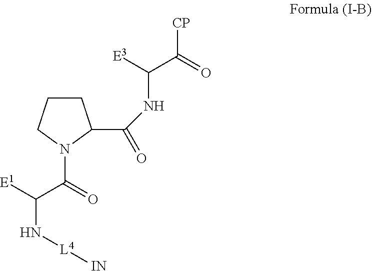
wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
L⁴ is a bivalent polyamine or polyamide linker;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃⁺, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and
IN is an integrin binder.

4. The compound of claim 1, or a pharmaceutically acceptable salt thereof, or a stereoisomer or mixture of stereoisomers thereof, having the structure of Formula (I-C1) or Formula (I-C2): 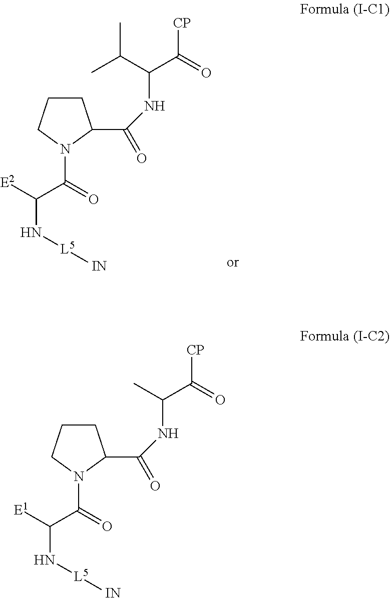
wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E² is —CH₂C(O)OR¹ or —CH₂CH₂C(O)OR¹;
L⁵ is a bivalent linker;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, -L⁶-MOD, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —NHL⁶-MOD, —N(C₁₋₆alkyl)₂, —N(C₁₋₆alkyl)₃⁺, —NHCO(C₁₋₆alkyl), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and
IN is an integrin binder.

5. The compound of claim 1, or a pharmaceutically acceptable salt thereof, or a stereoisomer or mixture of stereoisomers thereof, having the structure of Formula (I-D): 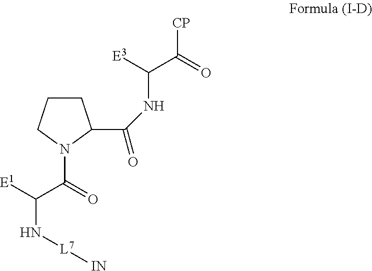
wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
L⁷ is a substituted or unsubstituted C₁₋₆₀ alkyl, or substituted or unsubstituted heteroalkyl comprising 4-12 heteroalkyl units, wherein each Z group is independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, and —NHS(O)₂NHC(O)—;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆alkyl)₃^{m}, —NHCO(C₁₋₆alkyl), —NHCO(IN), —N(C₁₋₆alkyl)CO(C₁₋₆alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and
IN is an integrin binder.

6. The compound of claim 1, or a pharmaceutically acceptable salt thereof, or a stereoisomer or mixture of stereoisomers thereof, having the structure of Formula (I-E): 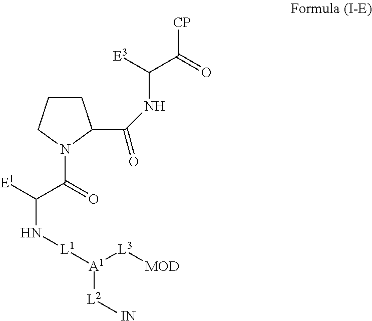
wherein:
CP is a cytotoxic or cytostatic group;
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E³ is —CH₃, —CH(CH₃)₂, —CH₂CH(CH₃)₂, or —CH(CH₃)CH₂CH₃;
A¹ is a trivalent linker;
each of L¹, L², and L³ is independently a bivalent linker;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and
MOD is a physicochemical or pharmacokinetic modulating group; and
IN is, in each instance, independently, an integrin binder.

7. The compound of any one of claims 1-6, or a pharmaceutically acceptable salt thereof, or a stereoisomer or mixture of stereoisomers thereof, wherein the compound is cleaved by neutrophil elastase.

8. The compound of any one of claims 1-7, or a pharmaceutically acceptable salt thereof, or a stereoisomer or mixture of stereoisomers thereof, wherein CP is a pentacyclic cytotoxic or cytostatic group.

9. The compound of any one of claims 1-8, or a pharmaceutically acceptable salt thereof, or a stereoisomer or mixture of stereoisomers thereof, wherein CP is a topoisomerase inhibitor.

10. The compound of any one of claims 1-9, or a pharmaceutically acceptable salt thereof, or a stereoisomer or mixture of stereoisomers thereof, wherein CP is bonded via an ester linkage.

11. The compound of any one of claims 1-10, or a pharmaceutically acceptable salt thereof, or a stereoisomer or mixture of stereoisomers thereof, wherein CP is a 7-ethylcamptothecin group.

12. The compound of any one of claims 1-11, or a pharmaceutically acceptable salt thereof, or a stereoisomer or mixture of stereoisomers thereof, having the structure of Formula (II): 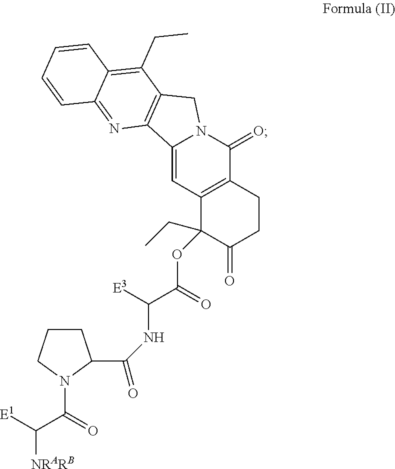
or a pharmaceutically acceptable salt thereof, or a stereoisomer or mixture of stereoisomers thereof, wherein each of E¹, E³, R^{A}, and R^{B} is as defined in claim 1.

13. The compound of any one of claims 1-12, or a pharmaceutically acceptable salt thereof, or a stereoisomer or mixture of stereoisomers thereof, having the structure of Formula (II-A), Formula (II-B), Formula (II-C), Formula (II-D), or Formula (II-E): 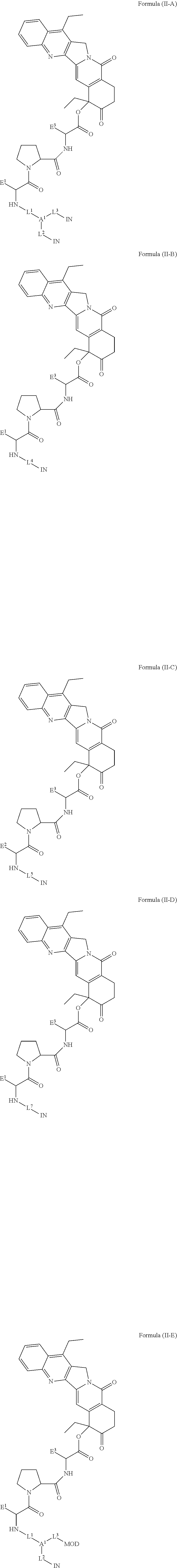
or a pharmaceutically acceptable salt thereof, or a stereoisomer or mixture of stereoisomers thereof, wherein:
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E² is —CH₂C(O)OR¹ or —CH₂CH₂C(O)OR¹;
E³ is —CH₃, —CH(CH₃)₂, —CH₂CH(CH₃)₂, or —CH(CH₃)CH₂CH₃;
IN is, in each instance, independently, an integrin binder;
A¹ is a trivalent linker;
each of L¹, L², L³, and L⁵ is independently a bivalent linker;
L⁴ is a bivalent polyamine or polyamide linker;
L⁷ is a substituted or unsubstituted C₁₋₆₀ alkyl, or substituted or unsubstituted heteroalkyl comprising 4-12 heteroalkyl units, wherein each Z group is independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, and —NHS(O)₂NHC(O)—;
R¹ is a substituted or unsubstituted alkyl, substituted or unsubstituted heteroalkyl, substituted or unsubstituted carbocycle, or a substituted or unsubstituted heterocycle; wherein if R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and IN is an integrin binder; and
MOD is a physicochemical or pharmacokinetic modulating group.

14. The compound of any one of claims 1-13, or a pharmaceutically acceptable salt thereof, or a stereoisomer or mixture of stereoisomers thereof, wherein IN is a linear peptide.

15. The compound of any one of claims 1-13, or a pharmaceutically acceptable salt thereof, or a stereoisomer or mixture of stereoisomers thereof, wherein IN is a macrocyclic peptide.

16. The compound of any one of claims 1-13, or a pharmaceutically acceptable salt thereof, or a stereoisomer or mixture of stereoisomers thereof, wherein IN is a constrained macrocyclic peptide.

17. The compound of any one of claims 1-13, or a pharmaceutically acceptable salt thereof, or a stereoisomer or mixture of stereoisomers thereof, wherein IN is a non-peptide integrin binder.

18. The compound of any one of claims 1-13, or a pharmaceutically acceptable salt thereof, or a stereoisomer or mixture of stereoisomers thereof, wherein IN is a small molecule integrin-binding moiety.

19. The compound of any one of claims 1-13, or a pharmaceutically acceptable salt thereof, or a stereoisomer or mixture of stereoisomers thereof, wherein IN is an αᵥβ₃ integrin binder.

20. The compound of any one of claims 1-13, or a pharmaceutically acceptable salt thereof, or a stereoisomer or mixture of stereoisomers thereof, wherein IN has the structure: 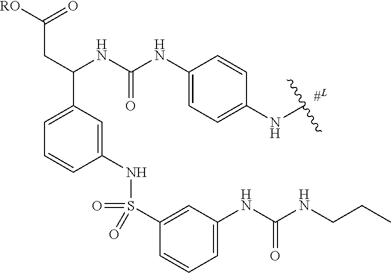
or a pharmaceutically acceptable salt thereof, or a stereoisomer thereof, wherein:
#^{L} denotes a bond to Lt, L², L³, L⁴, L⁵, L⁶, or L⁷; and
R is hydrogen or a substituted or unsubstituted C₁₋₁₂ alkyl.

21. The compound of any one of claims 1-13, or 17-20, or a pharmaceutically acceptable salt thereof, or a stereoisomer or mixture of stereoisomers thereof, having the structure of Formula (III-A), Formula (III-B), Formula (III-C), Formula (III-D), or Formula (III-E): 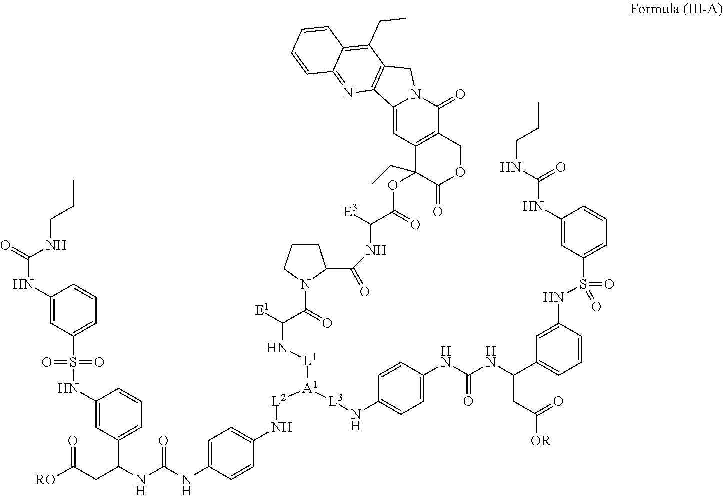 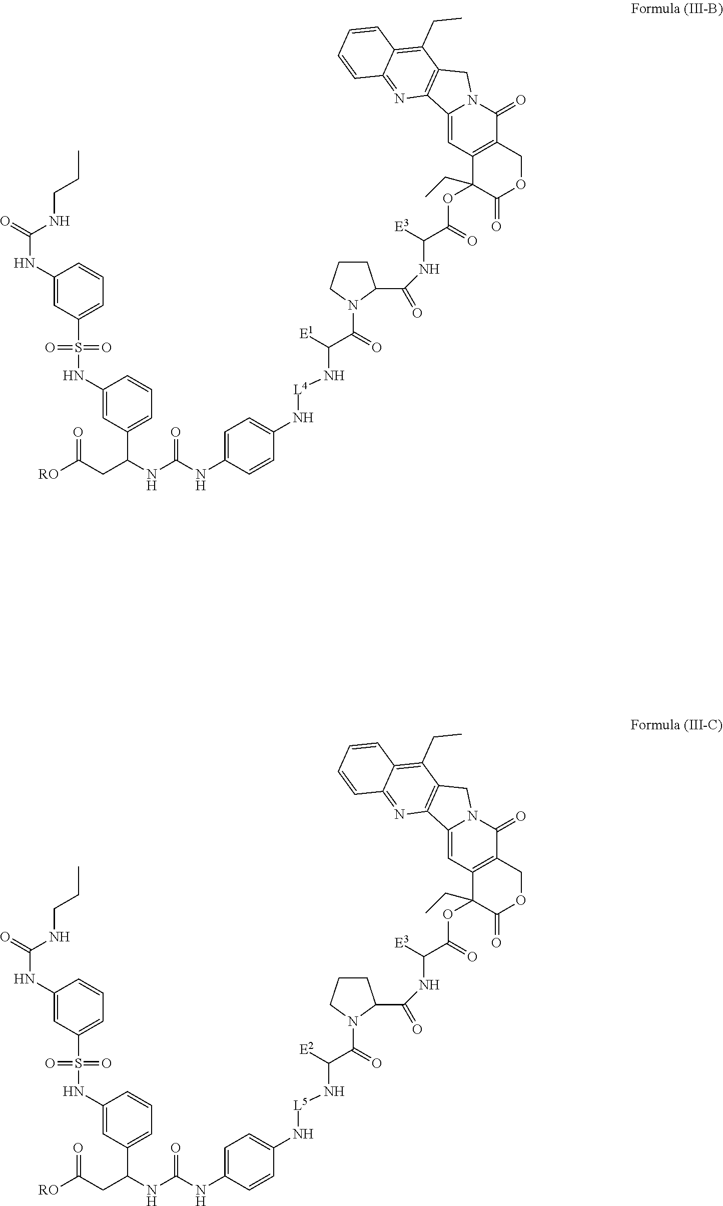 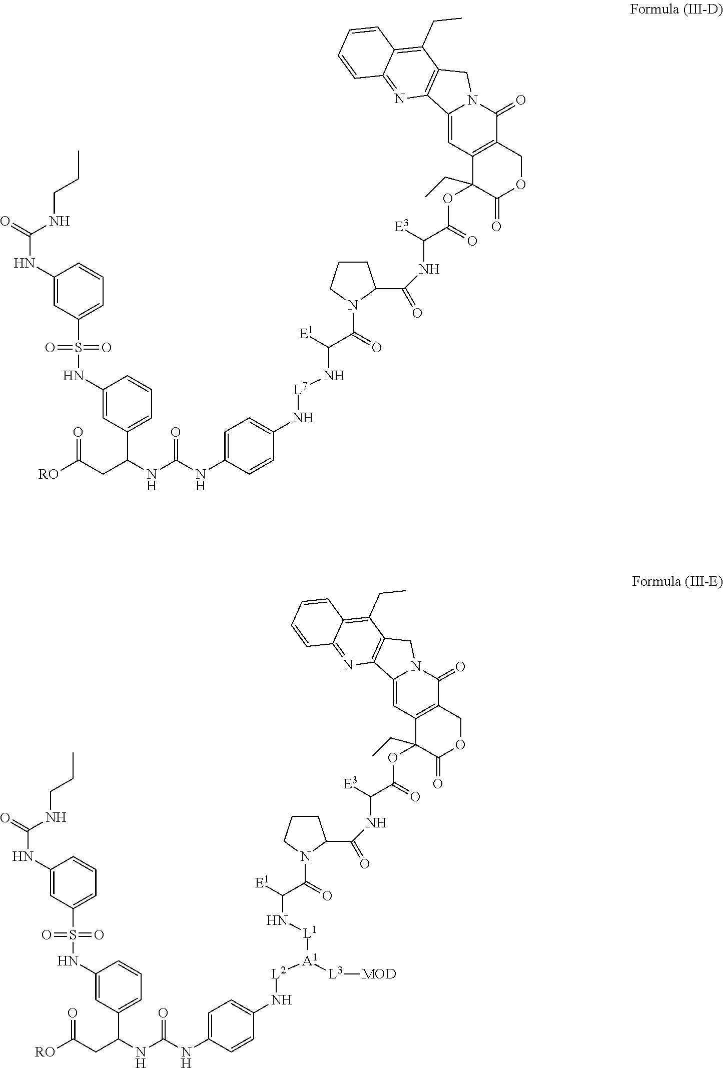
or a pharmaceutically acceptable salt thereof, or a stereoisomer or mixture of stereoisomers thereof, wherein:
E¹ is hydrogen, —CH₃, —CH₂CH₃, —CH₂CH₂CH₃, —CH₂C(O)NH₂, —CH₂C(O)OH, —CH₂C(O)OR¹, or —CH₂CH₂C(O)OR¹;
E² is —CH₂C(O)OR¹ or —CH₂CH₂C(O)OR¹;
E³ is —CH₃, —CH(CH₃)₂, —CH₂CH(CH₃)₂, or —CH(CH₃)CH₂CH₃;
IN is, in each instance, independently, an integrin binder;
A¹ is a trivalent linker;
each of L¹, L², L³, and L⁵ is independently a bivalent linker;
L⁴ is a bivalent polyamine or polyamide linker;
L⁷ is a substituted or unsubstituted C₁₋₆₀ alkyl, or substituted or unsubstituted heteroalkyl comprising 4-12 heteroalkyl units, wherein each Z group is independently selected from the group consisting of: —(O—C₂₋₆ alkyl)-, —(NH—C₁₋₆ alkyl)-, —(N(C₁₋₃ alkyl)-C₁₋₆ alkyl), —(N(C₁₋₃ alkyl)C(O)—C₁₋₆ alkyl), —NHS(O)₂NH—, and —NHS(O)₂NHC(O)—;
R is hydrogen or a substituted or unsubstituted C₁₋₁₂ alkyl; and
R¹ is a substituted or unsubstituted C₁₋₁₂ alkyl;
wherein if R or R¹ is substituted, it is substituted with one or more groups independently selected from deuterium, halogen, -L⁶-IN, —C₁₋₆alkyl, —CN, —CONH₂, —CONH(C₁₋₆ alkyl), —CON(C₁₋₆alkyl)₂, —COOH, —COO(C₁₋₆alkyl), —NH₂, —NH(C₁₋₆alkyl), —NHL⁶-IN, —N(C₁₋₆alkyl)₂, —N(C₁₋₆ alkyl)₃⁺, —NHCO(C₁₋₆alkyl), —NHCO(IN), —N(C₁₋₆ alkyl)CO(C₁₋₆ alkyl), —OH, —O(C₁₋₆alkyl), —OC(═O)O(C₁₋₆alkyl), —OC(═O)NH(C₁₋₆alkyl), oxo, —SO₃H, —SO₂(C₁₋₆alkyl), —SO₂NH₂, —SO₂NH(C₁₋₆alkyl), and —SO₂N(C₁₋₆alkyl)₂; wherein L⁶ is a substituted or unsubstituted C₁₋₃₀ alkyl, or substituted or unsubstituted heteroalkyl; and
MOD is a physicochemical or pharmacokinetic modulating group.

22. The compound of any one of claims 1-21, or a pharmaceutically acceptable salt thereof, or a stereoisomer or mixture of stereoisomers thereof, wherein each of L¹, L², L³, L⁴, L⁵, L⁶, and L⁷ contains or is terminally substituted with one or more carbonyl groups (—C(O)—), amine groups (e.g., —NH— or —N(CH₃)—), or amide groups (e.g., —C(O)NH—, —C(O)N(CH₃)—, —NHC(O)—, or N(CH₃)C(O)—).

23. The compound of any one of claims 1-22, or a pharmaceutically acceptable salt thereof, or a stereoisomer or mixture of stereoisomers thereof, wherein each of L¹, L², L³, and L⁵ is a substituted or unsubstituted C₂₋₂₀ alkyl chain that is optionally interrupted one or more times by groups selected from: —C(O)—, —C(O)NH—, —C(O)N(CH₃)—, —C(O)O—, —NH—, —N(CH₃)—, —NHC(O)—, —N(CH₃)C(O)—, —NHC(O)NH—, —NHS(O)₂NH—, —NHS(O)₂NHC(O)—, —NHS(O)₂NHC(O)O—, —O—, —S—, —S(O)—, —S(O)₂—, —S(O)₂NH—, —S(O)₂NHC(O)—, —S(O)₂NHC(O)NH—, —S(O)₂NHC(O)O—, carbocyclyl, heterocyclyl, aralkyl, heteroaralkyl, heteroalkyl-aryl, heteroalkyl-heteroaryl, or any combination thereof.

24. The compound of any one of claims 1-22, or a pharmaceutically acceptable salt thereof, or a stereoisomer or mixture of stereoisomers thereof, wherein one or more of L¹, L², L³, L⁴, L⁵, L, and L⁷ is an optionally substituted polyamine or polyamide linker.

25. The compound of any one of claims 1-24, or a pharmaceutically acceptable salt thereof, or a stereoisomer or mixture of stereoisomers thereof, wherein one or more of L¹, L², L³, and L⁵, is a bivalent polymeric linker of the formula:
—(CO)ₘ(CH₂)ₙ(OC₂₋₆ alkyl)ₒ(NH)ₚ(CO)_{q}—; (i)
—(CO)ᵣ(CH₂)ₛ(NR¹⁰C₁₋₆ alkyl)ₜ(NR¹¹)ᵤ(CO)ᵥ—; or (ii)
—(CO)ᵣ(CH₂)ₛ(NR¹⁰C(O)C₁₋₆ alkyl)ₜ(NR¹¹)ᵤ(CO)ᵥ—; (iii)
wherein:
R¹⁰ is hydrogen or C₁₋₃ alkyl;
R¹¹ is hydrogen or C₁₋₃ alkyl;
m is 0 or 1;
n is 0 to 10;
o is 1 to 10;
p is 0 or 1;
q is 0 or 1;
r is 0 or 1;
s is 0 to 10;
t is 1 to 10;
u is 0 or 1; and v is 0 or 1.

26. The compound of any one of claims 1-25, or a pharmaceutically acceptable salt thereof, or a stereoisomer or mixture of stereoisomers thereof, wherein L¹, L², L³, L⁵, or L⁶ is a bivalent linker selected from the group consisting of:
—C(O)—,
—C(O)NH—,
—C(O)N(CH₃)—,
—C₁₋₃₀ alkyl-,
—C(O)—C₁₋₆ alkyl-NHC(O)—
—C(O)—C₁₋₆ alkyl-N(CH₃)C(O)—
—C(O)—C₂₋₆ alkyl-[O—C₂₋₆ alkyl]₁₋₈-NHC(O)—,
—C(O)—C₂₋₆ alkyl-[O—C₂₋₆ alkyl]₁₋₈-N(CH₃)C(O)—,
—C(O)—C₁₋₆ alkyl-[NH—C₁₋₆ alkyl]₁₋₈-NHC(O)—,
—C(O)—C₁₋₆ alkyl-[NH—C₁₋₆ alkyl]₁₋₈-N(CH₃)C(O)—,
—C(O)—C₁₋₆ alkyl-[N(CH₃)—C₁₋₆ alkyl]₁₋₈-NHC(O)—,
—C(O)—C₁₋₆ alkyl-[N(CH₃)—C₁₋₆ alkyl]₁₋₈—N(CH₃)C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈—N(CH₃)C(O)—,
—C(O)—C₁₋₆ alkyl-NH—
—C(O)—C₁₋₆ alkyl-N(CH₃)—
—C(O)—C₂₋₆ alkyl-[O—C₂₋₆ alkyl]₁₋₈-NH—,
—C(O)—C₂₋₆ alkyl-[O—C₂₋₆ alkyl]₁₋₈-N(CH₃)—,
—C(O)—C₁₋₆ alkyl-[NH—C₁₋₆ alkyl]₁₋₈-NH—,
—C(O)—C₁₋₆ alkyl-[NH—C₁₋₆ alkyl]₁₋₈-N(CH₃)—,
—C(O)—C₁₋₆ alkyl-[N(CH₃)—C₁₋₆ alkyl]₁₋₈-NH—,
—C(O)—C₁₋₆ alkyl-[N(CH₃)—C₁₋₆ alkyl]₁₋₈-N(CH₃)—,
—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]₁₋₈-N(CH₃)—,
—C₁₋₆ alkyl-NH—C(O)—
—C₁₋₆ alkyl-N(CH₃)—C(O)—
—C₂₋₆ alkyl-[O—C₂₋₆ alkyl]₁₋₈-NHC(O)—,
—C₂₋₆ alkyl-[O—C₂₋₆ alkyl]₁₋₈-N(CH₃)C(O)—,
—C₁₋₆ alkyl-[NH—C₂₋₆ alkyl]₁₋₈-NHC(O)—,
—C₁₋₆ alkyl-[NH—C₂₋₆ alkyl]₁₋₈-N(CH₃)C(O)—,
—C₁₋₆ alkyl-[N(CH₃)—C₂₋₆ alkyl]₁₋₈-NHC(O)—,
—C₁₋₆ alkyl-[N(CH₃)—C₂₋₆ alkyl]₁₋₈-N(CH₃)C(O)—,
—C₁₋₆ alkyl-NH—
—C₁₋₆ alkyl-C—N(CH₃)—
—C₂₋₆ alkyl-[O—C₂₋₆ alkyl]₁₋₈-NH—,
—C₂₋₆ alkyl-[O—C₂₋₆ alkyl]₁₋₈-N(CH₃)—,
—C₁₋₆ alkyl-[NH—C₂₋₆ alkyl]₁₋₈-NH—,
—C₁₋₆ alkyl-[NH—C₂₋₆ alkyl]₁₋₈-N(CH₃)—,
—C₁₋₆ alkyl-[N(CH₃)—C₂₋₆ alkyl]₁₋₈-NH—, or
—C₁₋₆ alkyl-[N(CH₃)—C₂₋₆ alkyl]₁₋₈-N(CH₃)—.

27. The compound of any one of claims 1-24, or a pharmaceutically acceptable salt thereof, or a stereoisomer or mixture of stereoisomers thereof, wherein L⁴ is a bivalent polyamine or polyamide linker selected from the group consisting of:
—C(O)—C₁₋₆ alkyl-[NH—C₂₋₆ alkyl]₁₋₈-NHC(O)—,
—C(O)—C₁₋₆ alkyl-[NH—C₂₋₆ alkyl]₁₋₈-N(CH₃)C(O)—,
—C(O)—C₁₋₆ alkyl-[N(CH₃)—C₁₋₆ alkyl]₁₋₈-NHC(O)—,
—C(O)—C₁₋₆ alkyl-[N(CH₃)—C₁₋₆ alkyl]₁₋₈-N(CH₃)—C(O)—,
—C(O)—C₁₋₆ alkyl-[N(CH₃)C(O)—C₁₋₆ alkyl]₁₋₈-NHC(O)—,
—C(O)—C₁₋₆ alkyl-[N(CH₃)C(O)—C₁₋₆ alkyl]₁₋₈-N(CH₃)C(O)—,
—C(O)—C₁₋₆ alkyl-[NH—C₂₋₆ alkyl]₁₋₈-NH—,
—C(O)—C₁₋₆ alkyl-[NH—C₂₋₆ alkyl]₁₋₈-N(CH₃)—,
—C(O)—C₁₋₆ alkyl-[N(CH₃)—C₂₋₆ alkyl]₁₋₈-NH—,
—C(O)—C₁₋₆ alkyl-[N(CH₃)—C₂₋₆ alkyl]₁₋₈-N(CH₃)—,
—C(O)—C₁₋₆ alkyl-[N(CH₃)C(O)—C₁₋₆ alkyl]₁₋₈-NH—,
—C(O)—C₁₋₆ alkyl-[N(CH₃)C(O)—C₁₋₆ alkyl]₁₋₈-N(CH₃)—,
—C₁₋₆ alkyl-[NH—C₂₋₆ alkyl]₁₋₈-NHC(O)—,
—C₂₋₆ alkyl-[O—C₂₋₆ alkyl]₁₋₈-N(CH₃)C(O)—,
—C₁₋₆ alkyl-[N(CH₃)—C₂₋₆ alkyl]₁₋₈-NHC(O)—,
—C₁₋₆ alkyl-[N(CH₃)—C₂₋₆ alkyl]₁₋₈-N(CH₃)C(O)—,
—C₁₋₆ alkyl-[NH—C₂₋₆ alkyl]₁₋₈-NH—,
—C₁₋₆ alkyl-[NH—C₂₋₆ alkyl]₁₋₈-N(CH₃)—,
—C₁₋₆ alkyl-[N(CH₃)—C₂₋₆ alkyl]₁₋₈-NH—, or
—C₁₋₆ alkyl-[N(CH₃)—C₂₋₆ alkyl]₁₋₈-N(CH₃)—.

28. The compound of any one of claims 1-21, or a pharmaceutically acceptable salt thereof, or a stereoisomer or mixture of stereoisomers thereof, wherein L⁷ is:
—(CO)ₐ(C₀₋₆ alkyl)(Z¹C₁₋₆ alkyl)_{b}-Z²—(C₀₋₆ alkyl)(Z³C₁₋₆ alkyl)_{c}(Z⁴)_{d}(CO)ₑ—, or (i)
—(CO)ₐ(C₀₋₆ alkyl)(Z¹C(O)C₁₋₆ alkyl)_{b}-Z²—; (ii)
wherein:
Z¹ is in each occurrence independently —O—, —NH—, or —N(C₁₋₃ alkyl)-;
Z² is a bond, —C(O)—, —C(O)NH—, —C(O)—(C₁₋₆ alkyl)-, —C(O)—(C₁₋₆ alkyl)-C(O)—, —C(O)—(C₁₋₆ alkyl)-C(O)NH—, —C(O)—(C₁₋₆ alkyl)-C(O)N(CH₃)—, —C(O)NH—(C₁₋₆ alkyl)-C(O)NH—, —NH—, —N(C₁₋₆ alkyl)-, —N(C₁₋₆ alkyl)C(O)—, —NHC(O)—, —NHC(O)NH—, or —NHC(O)—(C₁₋₆ alkyl)-NHC(O)—;
Z³ is in each occurrence independently —O—, —NH—, or —N(C₁₋₃ alkyl)-;
Z⁴ is —O—, —NH—, or —N(C₁₋₃ alkyl)-;
a is 0 or 1;
b is 1 to 10;
c is 1 to 10;
d is 0 or 1; and
e is 0 or 1.

29. The compound of claim 28, or a pharmaceutically acceptable salt thereof, or a stereoisomer or mixture of stereoisomers thereof, wherein:
Z¹ is in each occurrence independently —O— or —N(CH₃)—;
Z² is —NHC(O)— or —N(C₁₋₆ alkyl)C(O)—;
Z³ is in each occurrence independently —O— or —N(CH₃)—;
Z⁴ is —NH—;
a is 1;
b is 2, 3, 4, 5, 6, 7, 8, 9, or 10;
c is 2, 3, 4, 5, 6, 7, 8, 9, or 10;
d is 1; and
e is 1.

30. The compound of any one of claims 1-29, or a pharmaceutically acceptable salt thereof, or a stereoisomer or mixture of stereoisomers thereof, wherein L⁷ is:
—C(O)—C₂₋₄ alkyl-[O—C₂₋₄ alkyl]₂₋₄-NHC(O)—C₂₋₄ alkyl-[O—C₂₋₄ alkyl]₂₋₄-NHC(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-NHC(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-NHC(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-NHC(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C alkyl-[N(CH₃)—C₂₋₄ alkyl]₂₋₄-NHC(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-NHC(O)—,
—C(O)—C₂₋₄ alkyl-[O—C₂₋₄ alkyl]₂₋₄-NHC(O)—C₂₋₄ alkyl-[O—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₂₋₄ alkyl-[O—C₂₋₄ alkyl]₂₋₄-N(CH₃)C(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-N(CH₃)C(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C(C)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)C(O)—,
—C(O)—C₁₋₄ alkyl-[N(CH₃)C(O)—C₁₋₄ alkyl]₂₋₄N(CH₃)C(O)—C₁₋₄ alkyl-[N(CH₃)C(O)—C₁₋₄ alkyl]₂₋₄-NHC(O)—,
—C(O)—C₁₋₄ alkyl-[N(CH₃)C(O)—C₁₋₄ alkyl]₂₋₄-N(CH₃)C(O)—C₁₋₄ alkyl-[N(CH₃)C(O)—C₁₋₄ alkyl]₂₋₄-N(CH₃)C(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₂₋₄ alkyl-[O—C₂₋₄ alkyl]₂₋₄-N(CH₃)C(O)—C₁₋₄ alkyl-[N(CH₃)—C₂₋₄ alkyl]₂₋₄-N(CH₃)C(O)—, or
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₂₋₄ alkyl-[O—C₂₋₄ alkyl]₂₋₄-N(CH₃)C(O)—.

31. The compound of any one of claims 1-30, or a pharmaceutically acceptable salt thereof, or a stereoisomer or mixture of stereoisomers thereof, wherein L⁷ is:
—C(O)—C₁₋₄ alkyl-[NH—C₂₋₄ alkyl]₂₋₄-NHC(O)—C₁₋₄ alkyl-[NH—C₂₋₄ alkyl]₂₋₄-NHC(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-NHC(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-N(CH₃)C(O)—,
—C(O)—C₁₋₄ alkyl-[NH—C₂₋₄ alkyl]₂₋₄-N(CH₃)C(O)—C₁₋₄ alkyl-[NH—C₂₋₄ alkyl]₂₋₄-N(CH₃)C(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-NHC(O)—,
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-N(CH₃)C(O)—,
—C(O)—C₁₋₄ alkyl-[N(CH₃)—C₂₋₄ alkyl]₂₋₄-N(CH₃)C(O)—C₁₋₄ alkyl-[N(CH₃)—C₂₋₄ alkyl]₂₋₄-N(CH₃)C(O)—;
—C(O)—C₁₋₄ alkyl-[NHC(O)—C₁₋₄ alkyl]₂₋₁₀-NHC(O)—,
—C(O)—C₁₋₄ alkyl-[NHC(O)—C₁₋₄ alkyl]₂₋₁₀-N(CH₃)C(O)—,
—C(O)—C₁₋₄ alkyl-[N(CH₃)C(O)—C₁₋₄ alkyl]₂₋₁₀-NHC(O)—, or
—C(O)—C₁₋₄ alkyl-[N(CH₃)C(O)—C₁₋₄ alkyl]₂₋₁₀-N(CH₃)C(O)—.

32. The compound of any one of claims 1-13, or 17-21, or a pharmaceutically acceptable salt thereof, or a stereoisomer or mixture of stereoisomers thereof, having the structure of Formula (III-A): 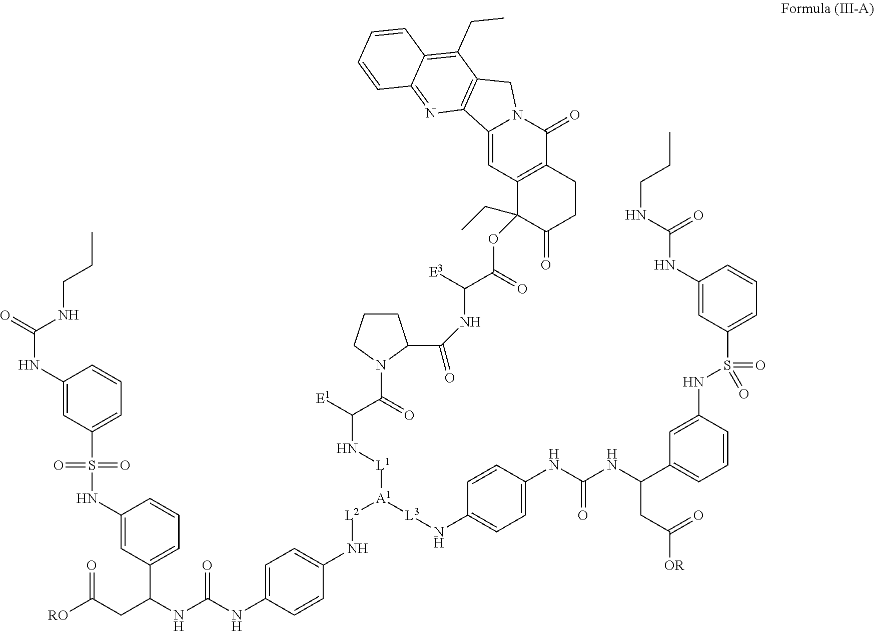
or a pharmaceutically acceptable salt thereof, or a stereoisomer or mixture of stereoisomers thereof.

33. The compound of claim 32, or a pharmaceutically acceptable salt thereof, or a stereoisomer or mixture of stereoisomers thereof, wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R is C₁₋₁₂ alkyl substituted with —NH₂, —N(C₁₋₆alkyl)₂, or —N(C₁₋₆alkyl)₃⁺;
R¹ is C₁₋₁₂ alkyl substituted with —NHL⁶-IN, —N(C₁₋₆alkyl)₂, or —N(C₁₋₆alkyl)₃⁺;
A¹ is 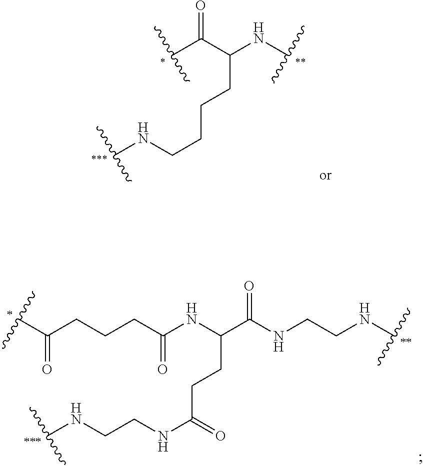 [SMILES: *NCCCCC(N*)C(*)=O ; *NCCNC(=O)CCC(NC(=O)CCCC(*)=O)C(=O)NCCN*]
wherein:
* is a bond between L and A¹;
** is a bond between A¹ and L²;
*** is a bond between A¹ and L³;
L¹ is —C(O)—C₂₋₆ alkyl-[O—C₂₋₆ alkyl]₁₋₈-NH*,
—C(O)—C₁₋₆ alkyl-[N(CH₃)—C₁₋₆ alkyl]₁₋₈-NH—*,
—C(O)—C₁₋₆ alkyl-[N(CH₃)—C₁₋₆ alkyl]₁₋₈-N(CH₃)—*; or
—C(O)—C₁₋₆ alkyl-[N(CH₃)C(O)—C₁₋₆ alkyl]₁₋₈-N(CH₃)—*;
L² is **C(O)—,
**C(O)—C₁₋₆ alkyl-NHC(O)—,
**C(O)—C₂₋₆ alkyl-[O—C₂₋₆ alkyl]₁₋₈-NHC(O)—,
**C(O)—C₁₋₆ alkyl-[N(CH₃)—C₂₋₆ alkyl]₁₋₈-NHC(O)—,
**C(O)—C₁₋₆ alkyl-[N(CH₃)—C₂₋₆ alkyl]₁₋₈-N(CH₃)C(O)—; or
**C(O)—C₁₋₆ alkyl-[N(CH₃)C(O)—C₁₋₆ alkyl]₁₋₈-N(CH₃)C(O)—;
L³ is ***C(O)—,
***C(O)—C₁₋₆ alkyl-NHC(O)—,
***C(O)—C₂₋₆ alkyl-[O—C₂₋₆ alkyl]₁₋₈-NHC(O)—,
***C(O)—C₁₋₆ alkyl-[N(CH₃)—C₂₋₆ alkyl]₁₋₈-NHC(O)—,
***C(O)—C₁₋₆ alkyl-[N(CH₃)—C₂₋₆ alkyl]₁₋₈-N(CH₃)C(O)—; or
***C(O)—C₁₋₆ alkyl-[N(CH₃)C(O)—C₁₋₆ alkyl]₁₋₈-N(CH₃)C(O)—; and
L⁶ is —C(O)—.

34. The compound of claim 33, or a pharmaceutically acceptable salt thereof, or a stereoisomer or mixture of stereoisomers thereof, wherein:
E¹ is hydrogen, —CH₂C(O)NH₂ or —CH₂C(O)OH;
E³ is —CH₃, or —CH(CH₃)₂;
L¹ is —C(O)—C₂₋₄ alkyl-[O—C₂₋₄ alkyl]₂₋₄-NH*,
—C(O)—C₁₋₄ alkyl-[N(CH₃)—C₁₋₄ alkyl]₂₋₄-NH—*,
—C(O)—C₁₋₄ alkyl-[N(CH₃)—C₁₋₄ alkyl]₂₋₄-N(CH₃)—*;
A¹ is 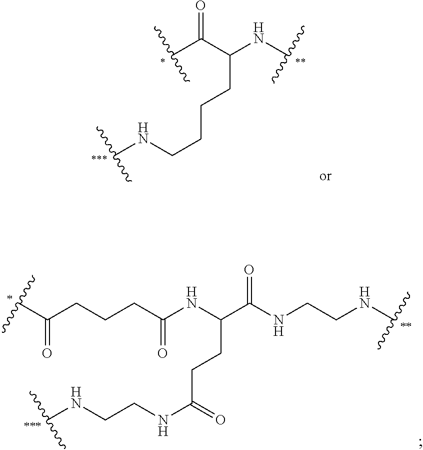 [SMILES: *NCCCCC(N*)C(*)=O ; *NCCNC(=O)CCC(NC(=O)CCCC(*)=O)C(=O)NCCN*]
L² is **C(O)—;
**C(O)—C₂₋₄ alkyl-NHC(O)—,
**C(O)—C₂₋₄ alkyl-[O—C₂₋₆ alkyl]₂₋₄-NHC(O)—,
**C(O)—C₁₋₄ alkyl-[N(CH₃)—C₁₋₆ alkyl]₂₋₄-NHC(O)—, or
**C(O)—C₁₋₄ alkyl-[N(CH₃)—C₁₋₆ alkyl]₂₋₄-N(CH₃)C(O)—; and
L³ is ***C(O)—,
***C(O)—C₂₋₄ alkyl-NHC(O)—,
***C(O)—C₂₋₄ alkyl-[O—C₂₋₆ alkyl]₂₋₄-NHC(O)—,
***C(O)—C₁₋₄ alkyl-[N(CH₃)—C₁₋₆ alkyl]₂₋₄-NHC(O)—, or
***C(O)—C₁₋₄ alkyl-[N(CH₃)—C₁₋₆ alkyl]₂₋₄-N(CH₃)C(O)—.

35. The compound of any one of claims 1, 2, 7-13, 17-26, or 32-34, or a pharmaceutically acceptable salt thereof, or a stereoisomer or mixture of stereoisomers thereof, having the structure: 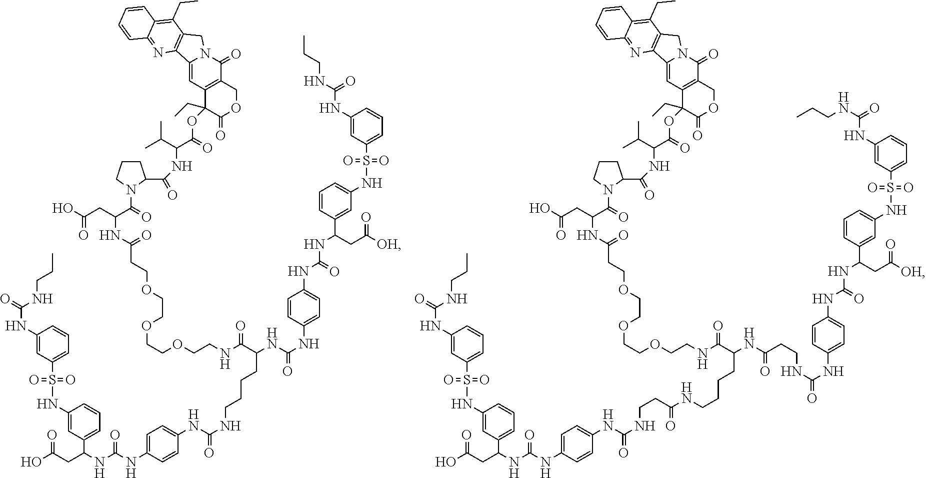 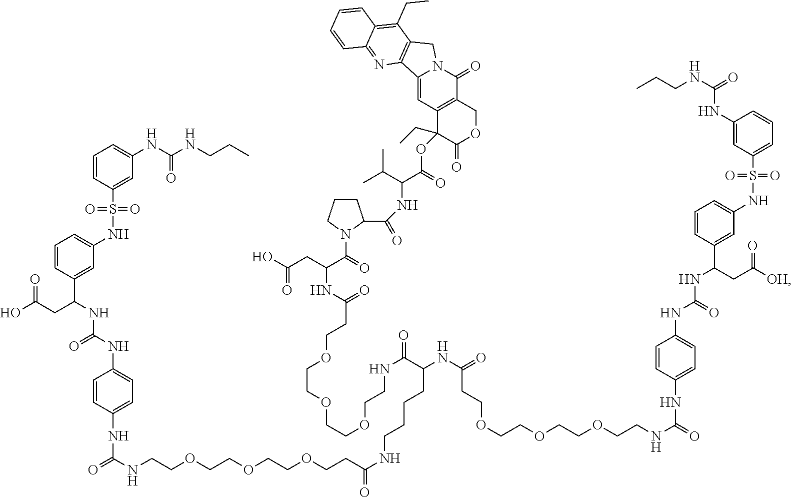 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCCCC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 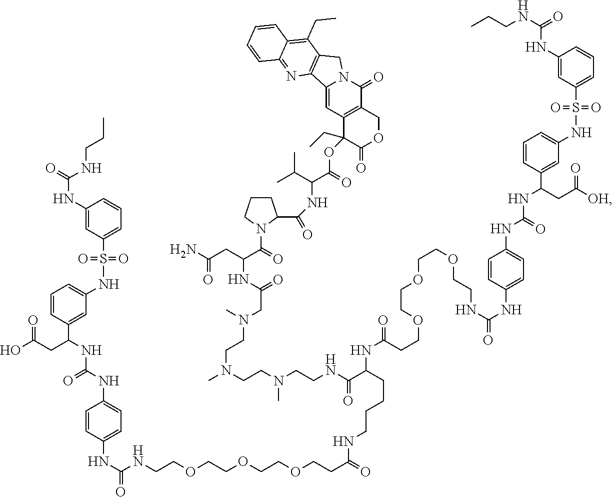 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCCCC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 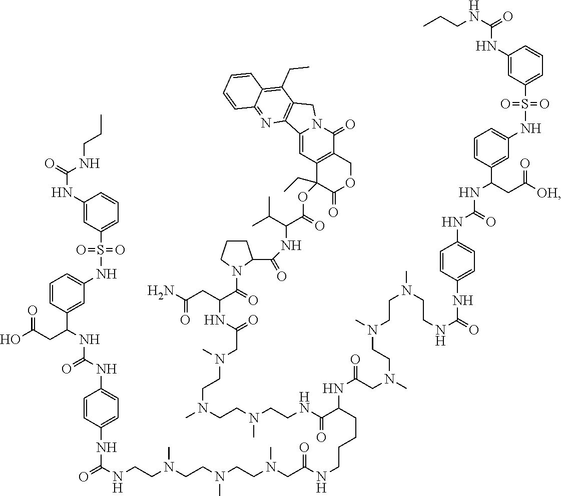 [SMILES: CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NCCCCC(NC(=O)CN(C)CCN(C)CCN(C)CCNC(=O)Nc4ccc(NC(=O)NC(CC(=O)O)c5cccc(NS(=O)(=O)c6cccc(NC(=O)NCCC)c6)c5)cc4)C(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 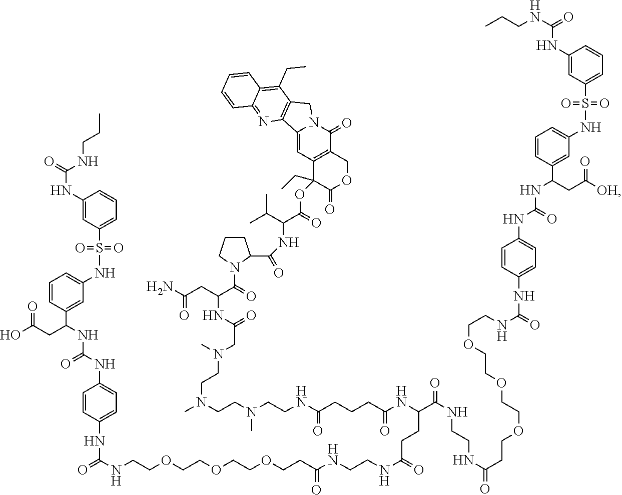 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCNC(=O)CCC(NC(=O)CCCC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4ccccc4c5CC)C(C)C)C(=O)NCCNC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)cc3)c2)c1] 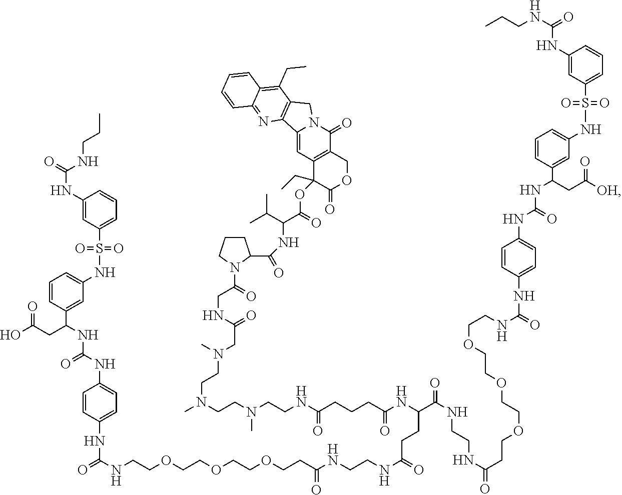 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCNC(=O)CCC(NC(=O)CCCC(=O)NCCN(C)CCN(C)CCN(C)CC(=O)NCC(=O)N4CCCC4C(=O)NC(C(=O)OC4(CC)C(=O)OCc5c4cc4n(c5=O)Cc5c-4nc4ccccc4c5CC)C(C)C)C(=O)NCCNC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)cc3)c2)c1] 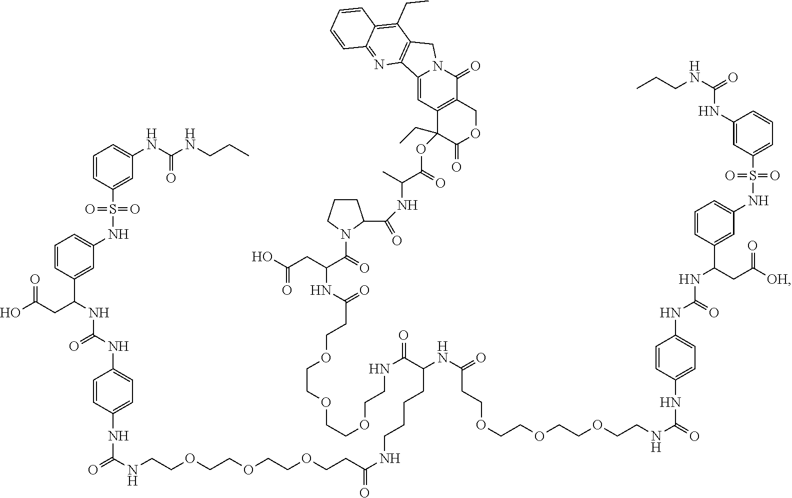 [SMILES: CCCNC(=O)Nc1cccc(S(=O)(=O)Nc2cccc(C(CC(=O)O)NC(=O)Nc3ccc(NC(=O)NCCOCCOCCOCCC(=O)NCCCCC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)NCCOCCOCCOCCC(=O)NC(CC(=O)O)C(=O)N4CCCC4C(=O)NC(C)C(=O)OC4(CC)C(=O)OCc5c4cc4n(c5=O)Cc5c-4nc4ccccc4c5CC)cc3)c2)c1]
or a pharmaceutically acceptable salt thereof, or a stereoisomer or mixture of stereoisomers thereof.

36. The compound of any one of claims 1-13, or 17-21, or a pharmaceutically acceptable salt thereof, or a stereoisomer or mixture of stereoisomers thereof, having the structure of Formula (III-B): 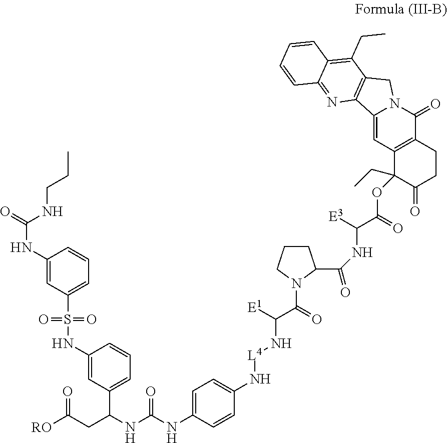
wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R is C₁₋₁₂ alkyl substituted with —NH₂, —N(C₁₋₆alkyl)₂, or —N(C₁₋₆alkyl)₃⁺;
R¹ is C₁₋₁₂ alkyl substituted with —NHL⁶-IN, —N(C₁₋₆alkyl)₂, or —N(C₁₋₆alkyl)₃⁺;
L⁴ is a bivalent polyamine or polyamide linker of the formula:
—(CO)ᵣ—(CH₂)ₛ—(NR¹⁰—C₁₋₆ alkyl)ₜ-(NR¹¹)ᵤ—(CO)ᵥ—; or
—(CO)ᵣ—(CH₂)ₛ—(NR¹⁰C(O)C₁₋₆ alkyl)ₜ-(NR¹¹)ᵤ—(CO)ᵥ—;
wherein:
R¹⁰ is hydrogen or C₁₋₃ alkyl;
R¹¹ is hydrogen or C₁₋₃ alkyl;
r is 0 or 1;
s is 0-10;
t is 1-10;
u is 0 or 1;
v is 0 or 1;
L⁶ is —C(O)—; and
IN is an integrin binder.

37. The compound of claim 36, or a pharmaceutically acceptable salt thereof, or a stereoisomer or mixture of stereoisomers thereof, wherein:
E¹ is hydrogen, —CH₂C(O)NH₂, or —CH₂C(O)OH; and
E³ is —CH₃ or —CH(CH₃)₂; and
L⁴ is —C(O)—C₁₋₆ alkyl-[N(CH₃)—C₁₋₆ alkyl]₁₋₈-NHC(O)—,
—C(O)—C₁₋₆ alkyl-[N(CH₃)—C₁₋₆ alkyl]₁₋₈-N(CH₃)C(O)—;
—C(O)—C₁₋₆ alkyl-[N(CH₃)C(O)—C₁₋₆ alkyl]₁₋₈-NHC(O)—, or
—C(O)—C₁₋₆ alkyl-[N(CH₃)C(O)—C₁₋₆ alkyl]₁₋₈-N(CH₃)C(O)—.

38. The compound of claim 37, or a pharmaceutically acceptable salt thereof, or a stereoisomer or mixture of stereoisomers thereof, wherein:
E¹ is hydrogen, —CH₂C(O)NH₂, or —CH₂C(O)OH; and
E³ is —CH(CH₃)₂;
L⁴ is —C(O)—C₁₋₄ alkyl-[N(CH₃)—C₁₋₄ alkyl]₂₋₄-NHC(O)—,
—C(O)—C₁₋₄ alkyl-[N(CH₃)—C₁₋₄ alkyl]₂₋₄-N(CH₃)C(O)—, or
—C(O)—C₁₋₄ alkyl-[N(CH₃)C(O)—C₁₋₄ alkyl]₂₋₄-N(CH₃)C(O)—.

39. The compound ofany one of claims 1, 3, 7-13, 17-27, or 36-38, or a pharmaceutically acceptable salt thereof, or a stereoisomer or mixture of stereoisomers thereof, having the structure: 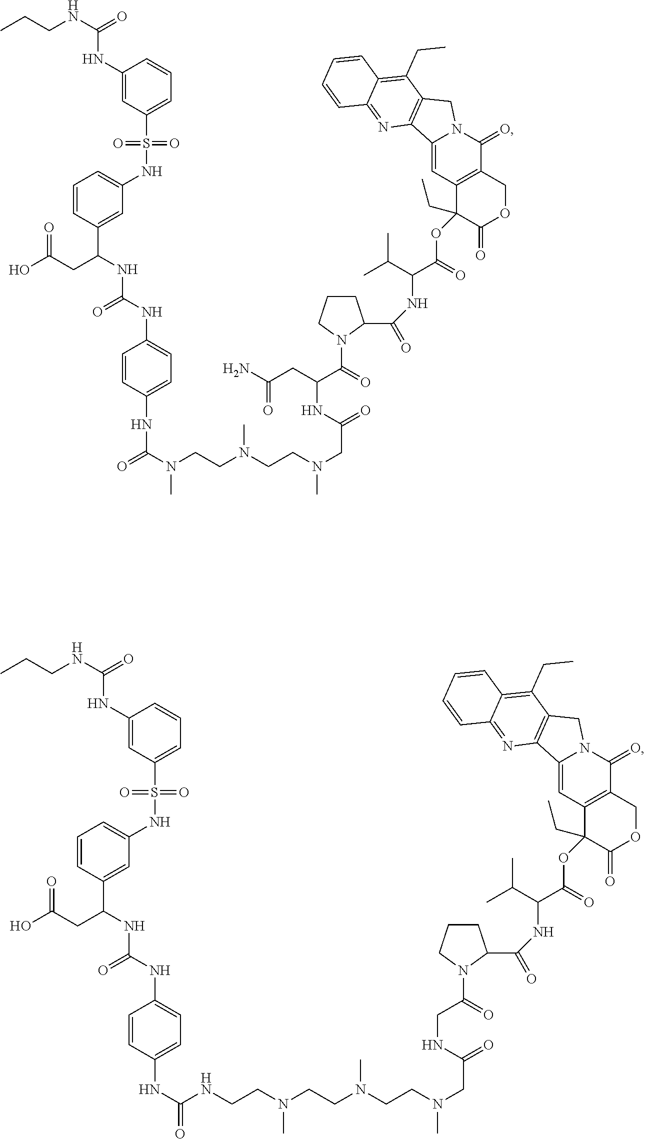 [SMILES: CCCNC(=O)Nc1cccc(S(=O)(=O)Nc2cccc(C(CC(=O)O)NC(=O)Nc3ccc(NC(=O)N(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NCC(=O)N4CCCC4C(=O)NC(C(=O)OC4(CC)C(=O)OCc5c4cc4n(c5=O)Cc5c-4nc4ccccc4c5CC)C(C)C)cc3)c2)c1] 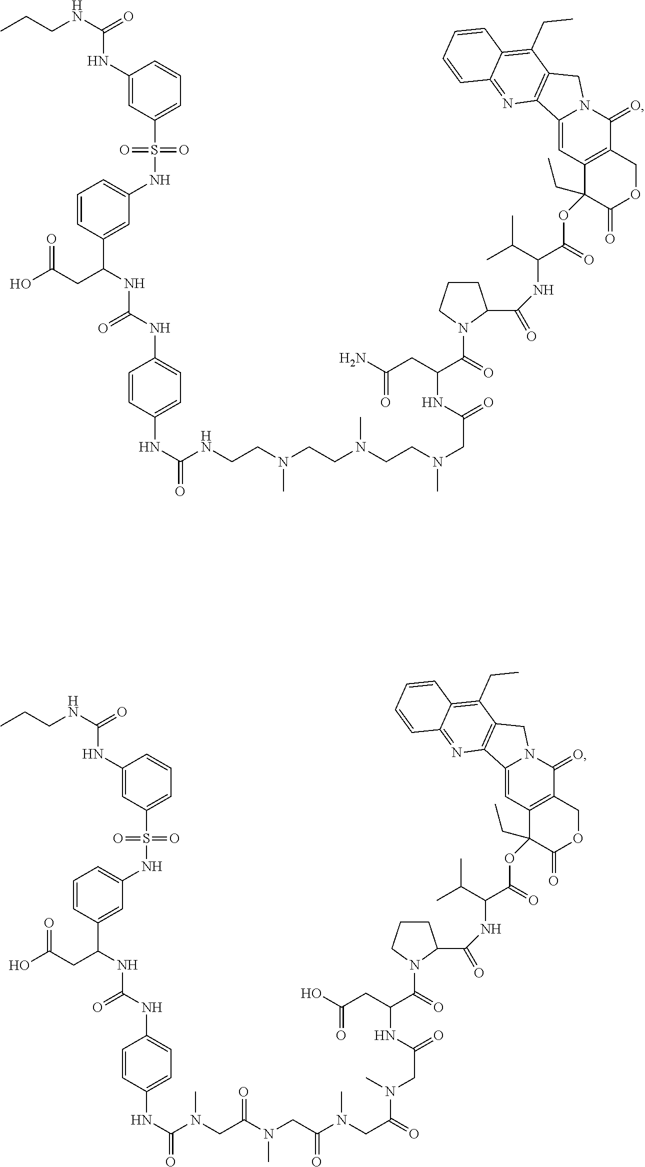 [SMILES: CCCNC(=O)Nc1cccc(S(=O)(=O)Nc2cccc(C(CC(=O)O)NC(=O)Nc3ccc(NC(=O)NCCN(C)CCN(C)CCN(C)CC(=O)NC(CC(N)=O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)N(C)CC(=O)N(C)CC(=O)N(C)CC(=O)N(C)CC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]
or a pharmaceutically acceptable salt thereof, or a stereoisomer or mixture of stereoisomers thereof.

40. The compound of any one of claims 1-13, or 17-21, or a pharmaceutically acceptable salt thereof, or a stereoisomer or mixture of stereoisomers thereof, having the structure of Formula (III-C): 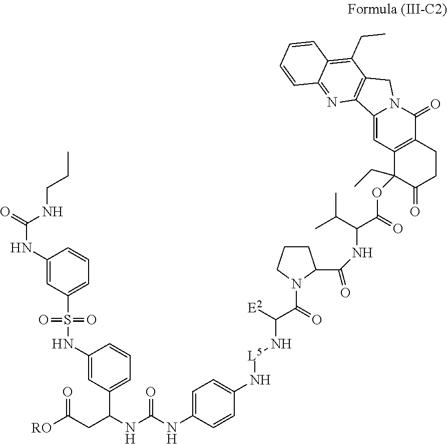
wherein:
E² is —CH₂C(O)OR¹ or —CH₂CH₂C(O)OR¹;
L⁵ is a linker having a structure represented by the formula:
—(CO)ₘ—(CH₂)ₙ—(OC₂₋₆ alkyl)ₒ-(NH)ₚ—(CO)_{q}—; (i)
—(CO)ᵣ—(CH₂)ₛ—(NR¹⁰C₁₋₆ alkyl)ₜ-(NR¹¹))ᵤ—(CO)ᵥ—; or (ii)
—(CO)ᵣ—(CH₂)ₛ—(NR¹⁰C(O)C₁₋₆ alkyl)ₜ-(NR¹¹)ᵤ—(CO)ᵥ—; (iii)
wherein:
each R¹⁰ and R¹¹ is independently hydrogen or C₁₋₃ alkyl;
each m, p, q, r, u, and v is independently 0 or 1; and
each n, o, s, and t is independently 0, 1, 2, 3, 4, 5, 6, 7, 8, 9, or 10.
R is substituted or unsubstituted C₁₋₁₂ alkyl;
R¹ is substituted or unsubstituted C₁₋₁₂ alkyl;
wherein if R or R¹ is substituted, it is substituted with one or more groups independently selected from -L⁶-IN, —C₁₋₆alkyl, —NH₂, —NH(C₁₋₆alkyl), —NHL⁶-IN, —N(C₁₋₆alkyl)₂, —N(C₁₋₆ alkyl)₃⁺, and —NHCO(IN); wherein L⁶ is a substituted or unsubstituted C₁₋₃₀ alkyl, or substituted or unsubstituted heteroalkyl; and IN is an integrin binder.

41. The compound of claim 40, or a pharmaceutically acceptable salt thereof, or a stereoisomer or mixture of stereoisomers thereof, wherein:
E² is —CH₂C(O)OR^{t} or —CH₂CH₂C(O)OR¹;
L⁵ is —C(O)—C₂₋₆ alkyl-[O—C₂₋₆ alkyl]₁₋₈-NHC(O)—,
—C(O)—C₁₋₆ alkyl-[NH—C₁₋₆ alkyl]₁₋₈-NHC(O)—,
—C(O)—C₁₋₆ alkyl-[N(CH₃)—C₁₋₆ alkyl]₁₋₈-NHC(O)—, or
—C(O)—C₁₋₆ alkyl-[N(CH₃)—C₁₋₆ alkyl]₁₋₈-N(CH₃)—C(O)—;
R is C₁₋₁₂ alkyl substituted with —NH₂, —N(C₁₋₆alkyl)₂, or —N(C₁₋₆alkyl)₃⁺;
R¹ is C₁₋₁₂ alkyl substituted with —NHL⁶-IN, —NH₂, —N(C₁₋₆alkyl)₂, or —N(C₁₋₆alkyl)₃⁺;
wherein L⁶ is —C(O)—; and IN is an integrin binder.

42. The compound of claim 40 or 41, or a pharmaceutically acceptable salt thereof, or a stereoisomer or mixture of stereoisomers thereof, wherein:
E² is —CH₂C(O)OR¹ or —CH₂CH₂C(O)OR¹;
L⁵ is —C(O)—C₂₋₄ alkyl-[O—C₂₋₄ alkyl]₂₋₄-NHC(O)—,
—C(O)—C₁₋₄ alkyl-[N(CH₃)—C₁₋₄ alkyl]₂₋₄-NHC(O)—, or
—C(O)—C₁₋₄ alkyl-[N(CH₃)C(O)—C₁₋₄ alkyl]₂₋₄-N(CH₃)C(O)—;
R is C₂₋₆ alkyl-NH₂, C₂₋₆ alkyl-N(CH₃)₂, or C₂₋₆ alkyl-N(CH₃)₃⁺,
R¹ is C₂₋₆ alkyl-NH₂, C₂₋₆ alkyl-N(CH₃)₂, C₂₋₆ alkyl-N(CH₃)₃⁺, or C₂₋₆ alkyl-NHC(O)IN; wherein IN is an integrin binder.

43. The compound of any one of claims 1, 4, 7-13, 17-26, or 40-42, or a pharmaceutically acceptable salt thereof, or a stereoisomer or mixture of stereoisomers thereof, having the structure: 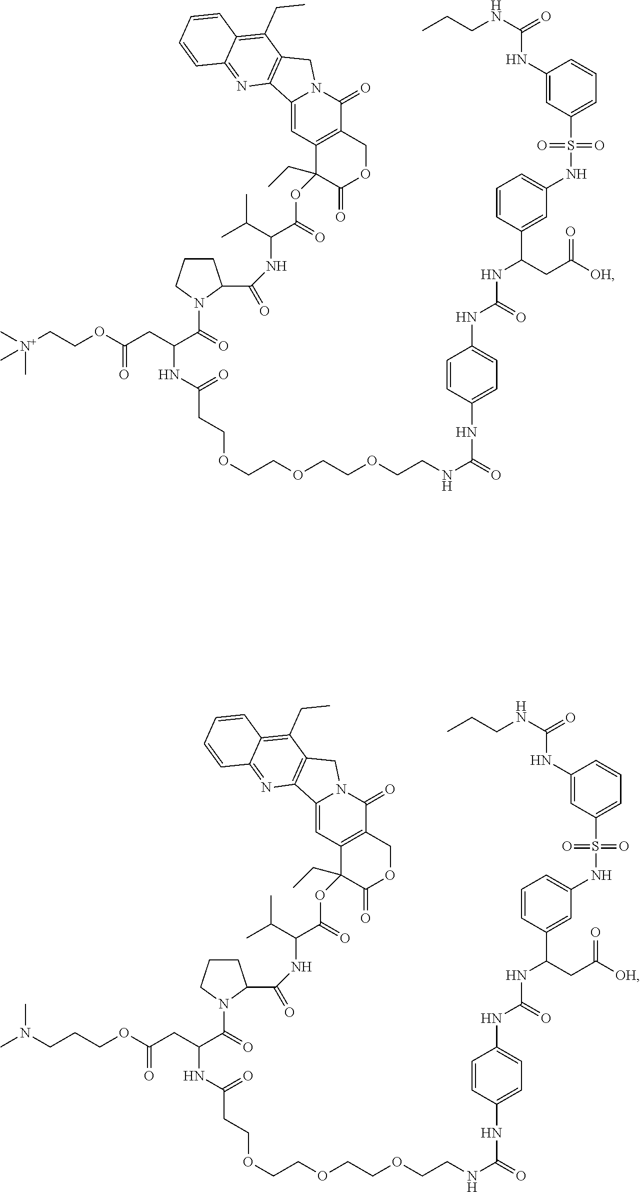 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCC[N+](C)(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCCN(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 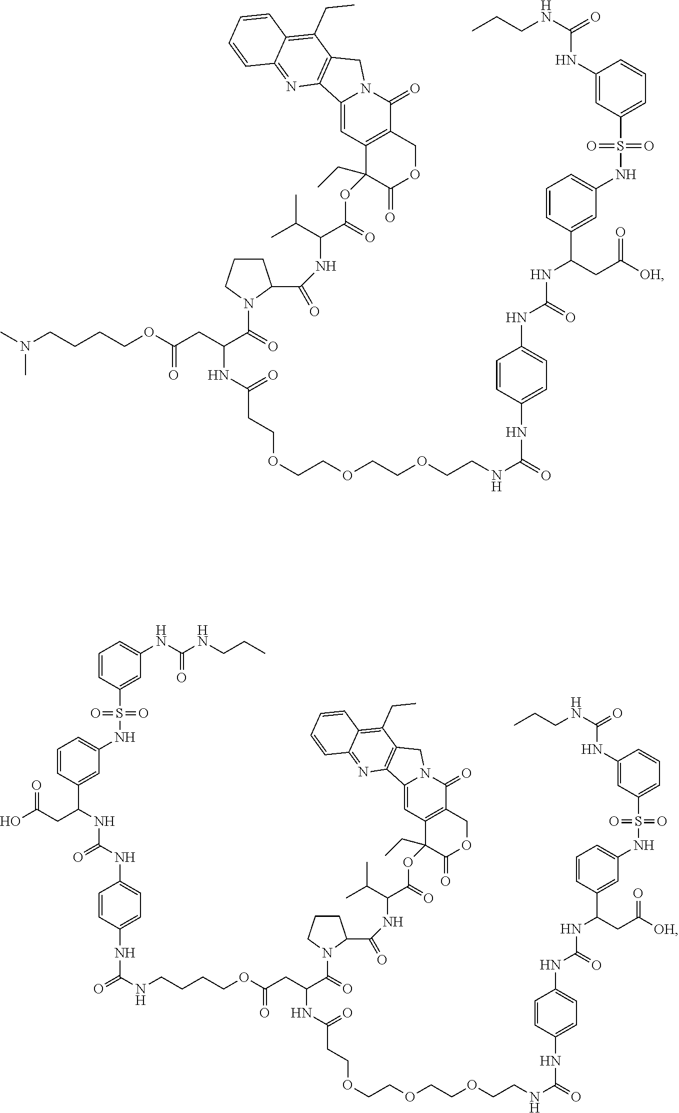 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCCCN(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)O)NC(=O)Nc3ccc(NC(=O)NCCCCOC(=O)CC(NC(=O)CCOCCOCCOCCNC(=O)Nc4ccc(NC(=O)NC(CC(=O)O)c5cccc(NS(=O)(=O)c6cccc(NC(=O)NCCC)c6)c5)cc4)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 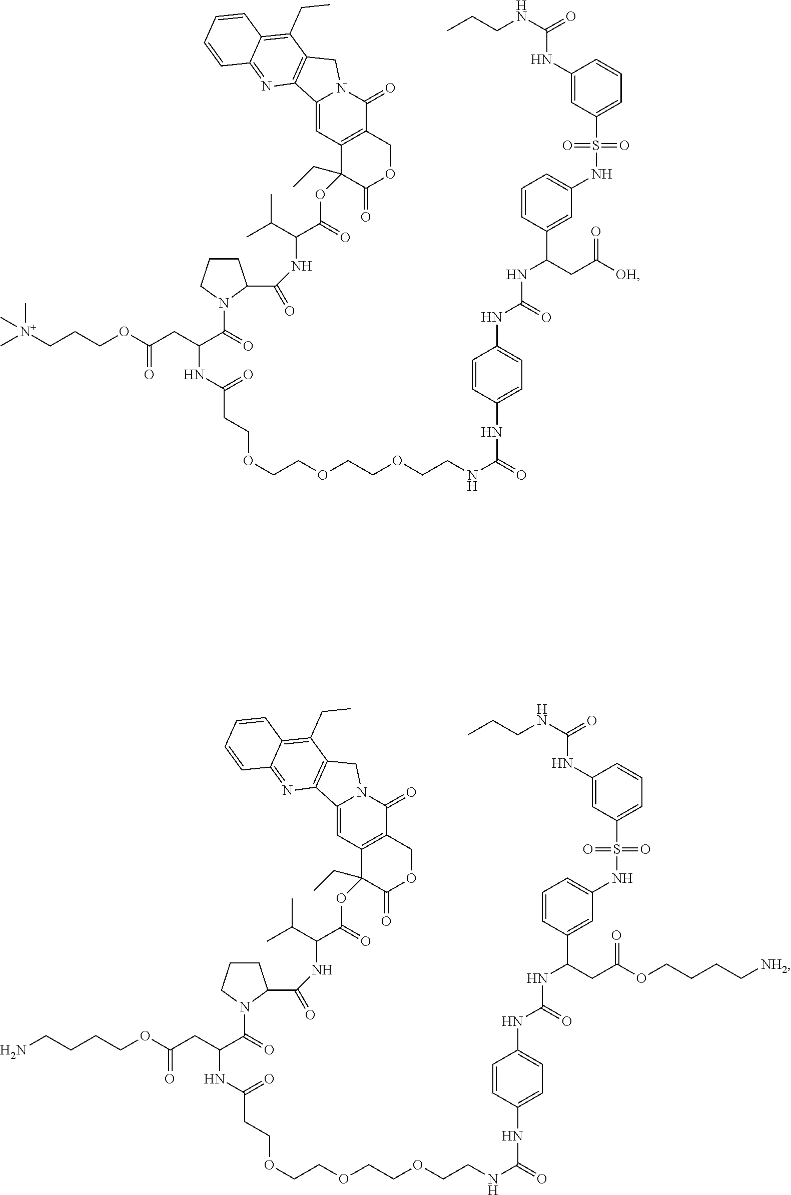 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)OCCC[N+](C)(C)C)C(=O)N4CCCC4C(=O)NC(C(=O)OC4(CC)C(=O)OCc5c4cc4n(c5=O)Cc5c-4nc4ccccc4c5CC)C(C)C)cc3)c2)c1 ; CCCNC(=O)Nc1cccc(S(=O)(=O)Nc2cccc(C(CC(=O)OCCCCN)NC(=O)Nc3ccc(NC(=O)NCCOCCOCCOCCC(=O)NC(CC(=O)OCCCCN)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 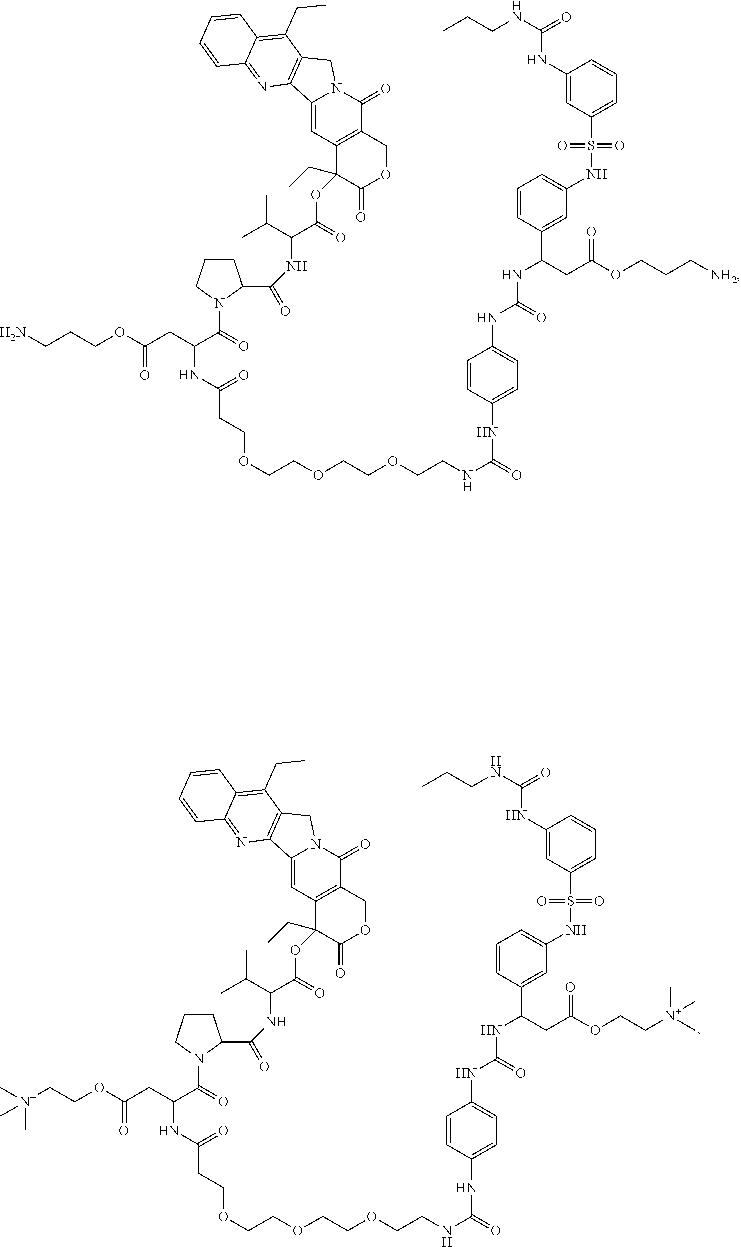
or a pharmaceutically acceptable salt thereof, or a stereoisomer or mixture of stereoisomers thereof.

44. The compound of any one of claims 1-13, or 17-21, or a pharmaceutically acceptable salt thereof, or a stereoisomer or mixture of stereoisomers thereof, having the structure: 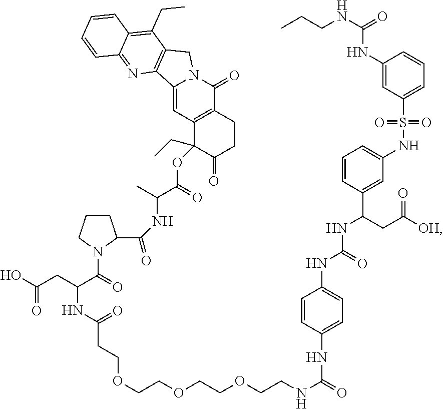 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CC(=O)O)C(=O)N4CCCC4C(=O)NC(C)C(=O)OC4(CC)C(=O)CCc5c4cc4n(c5=O)Cc5c-4nc4ccccc4c5CC)cc3)c2)c1]
or a pharmaceutically acceptable salt thereof, or a stereoisomer or mixture of stereoisomers thereof.

45. The compound of any one of claims 1-13, or 17-21, or a pharmaceutically acceptable salt thereof, or a stereoisomer or mixture of stereoisomers thereof, having the structure of Formula (III-D): 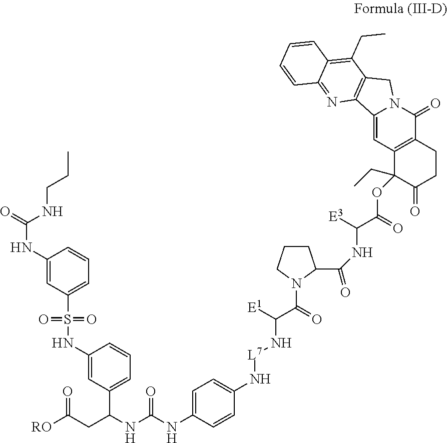
wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R is C₁₋₁₂ alkyl substituted with —NH₂, —N(C₁₋₆alkyl)₂, or —N(C₁₋₆alkyl)₃⁺;
Rt is C₁₋₁₂ alkyl substituted with —NHL⁶-IN, —N(C₁₋₆alkyl)₂, or —N(C₁₋₆alkyl)₃⁺;
L⁶ is —C(O)—; and
L⁷ is —C(O)—C₁₋₄ alkyl-[N(CH₃)C(O)—C₁₋₄ alkyl]₂₋₁₀-N(CH₃)C(O)—,
—C(O)—C₂₋₄ alkyl-[O—C₂₋₄ alkyl]₂₋₄-NHC(O)—C₂₋₄ alkyl-[O—C₂₋₄ alkyl]₂₋₄-NHC(O)—,
—C(O)—C₁₋₄ alkyl-[NH—C₂₋₄ alkyl]₂₋₄-NHC(O)—C₁₋₄ alkyl-[NH—C₂₋₄ alkyl]₂₋₄-NHC(O)—;
—C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—,
—C(O)—C₂₋₄ alkyl-[O—C₂₋₄ alkyl]₂₋₄-NHC(O)—C₁₋₄ alkyl-[N(CH₃)—C₂₋₄ alkyl]₂₋₄-NHC(O)—, or
—C(O)—C₁₋₄ alkyl-[N(CH₃)—C₂₋₄ alkyl]₂₋₄-NHC(O)—C₂₋₄ alkyl-[O—C₂₋₄ alkyl]₂₋₄-NHC(O)—.

46. The compound of claim 45, or a pharmaceutically acceptable salt thereof, or a stereoisomer or mixture of stereoisomers thereof, wherein E¹ is —CH₂C(O)OH or —CH₂C(O)NH₂; and L⁷ is —C(O)—C₁₋₄ alkyl-[N(CH₃)C(O)—C₁₋₄ alkyl]₂₋₁₀-N(CH₃)C(O)— or —C(O)—C₁₋₄ alkyl-[N(CH₃)—C₂₋₄ alkyl]₂₋₄-NHC(O)—C₁₋₄ alkyl-[N(CH₃)—C₂₋₄ alkyl]₂₋₄-NHC(O)—.

47. The compound of claim 45 or 46, or a pharmaceutically acceptable salt thereof, or a stereoisomer or mixture of stereoisomers thereof, wherein L⁷ is: 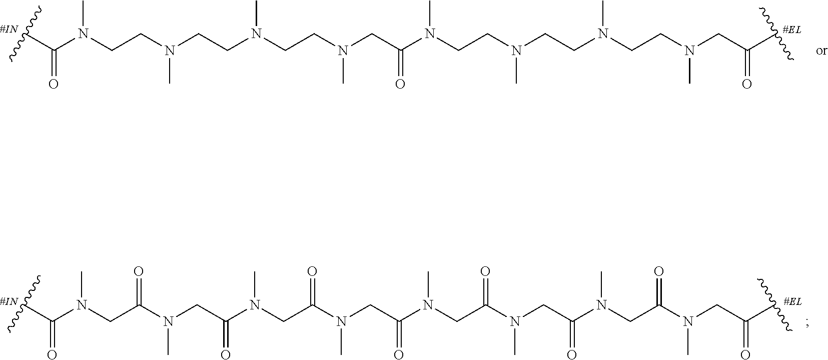 [SMILES: *C(=O)CN(C)CCN(C)CCN(C)CCN(C)C(=O)CN(C)CCN(C)CCN(C)CCN(C)C(*)=O ; *C(=O)CN(C)C(=O)CN(C)C(=O)CN(C)C(=O)CN(C)C(=O)CN(C)C(=O)CN(C)C(=O)CN(C)C(=O)CN(C)C(*)=O]
wherein #EL denotes a bond to EL; and #IN denotes a bond to IN.

48. The compound of any one of claims 1, 5, 7-13, 17-26, 28-31, or 45-47, or a pharmaceutically acceptable salt thereof, or a stereoisomer or mixture of stereoisomers thereof, having the structure: 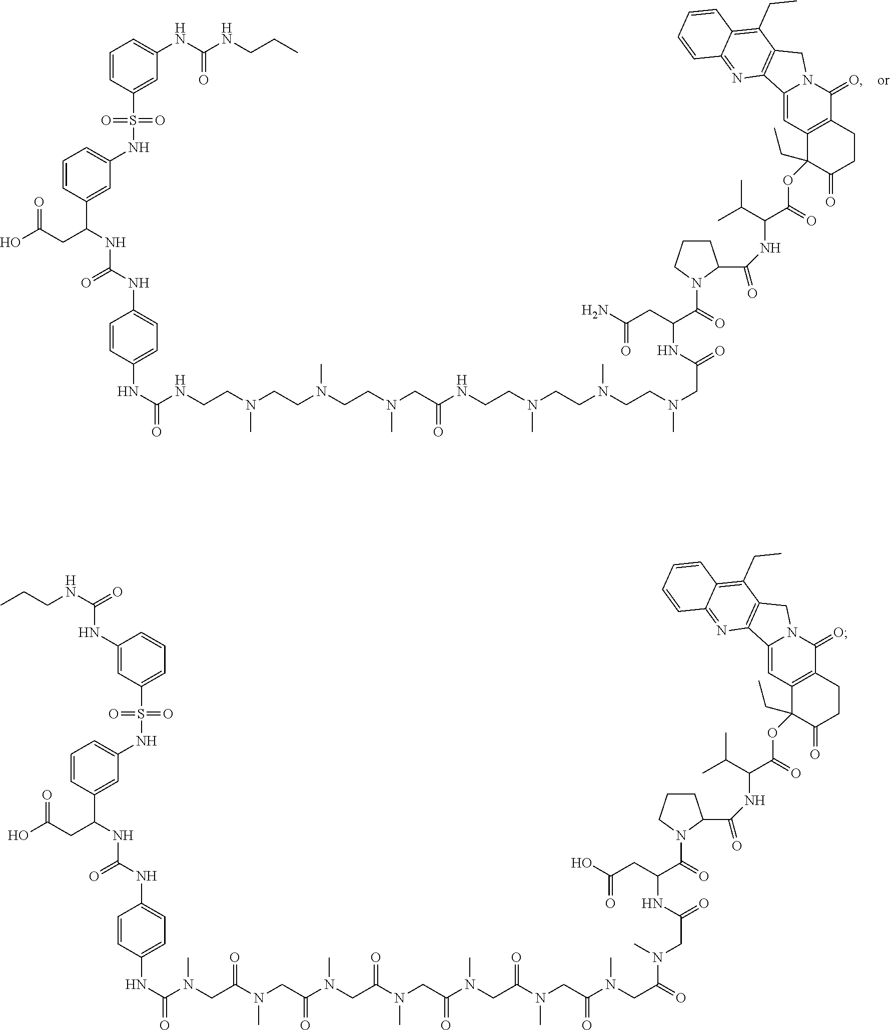
or a pharmaceutically acceptable salt thereof, or a stereoisomer or mixture of stereoisomers thereof.

49. The compound of any one of claims 1-13, or 17-21, or a pharmaceutically acceptable salt thereof, or a stereoisomer or mixture of stereoisomers thereof, having the structure of Formula (III-E): 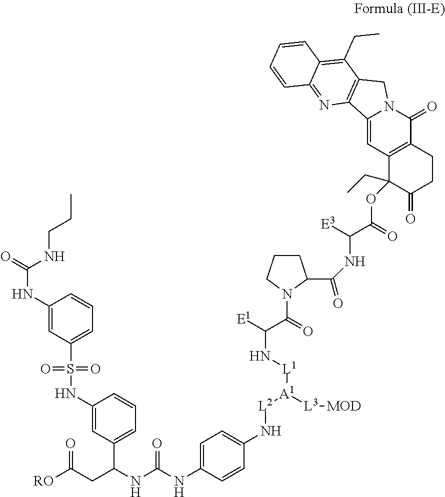
wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
R¹ is C₁₋₁₂ alkyl substituted with —NHL⁶-IN, —N(C₁₋₆alkyl)₂, or —N(C₁₋₆alkyl)₃⁺;
each L¹, L², L³, and L⁶ is independently a bivalent linker;
A¹ is a trivalent linker; and
MOD is —COOH.

50. The compound of claim 49, or a pharmaceutically acceptable salt thereof, or a stereoisomer or mixture of stereoisomers thereof, wherein:
E¹ is hydrogen, —CH₂C(O)NH₂, —CH₂C(O)OH, —CH₂C(O)OR¹;
E³ is —CH₃, —CH(CH₃)₂, —CH₂CH(CH₃)₂, or —CH(CH₃)CH₂CH₃;
L¹ is —C(O)—C₁₋₆ alkyl-[O—C₂₋₆ alkyl]₁₋₈-NH*,
—C(O)—C₁₋₆ alkyl-[N(CH₃)—C₁₋₆ alkyl]₁₋₈-NH—*,
—C(O)—C₁₋₆ alkyl-[N(CH₃)C(O)—C₁₋₆ alkyl]₁₋₈-NH—*,
—C(O)—C₁₋₆ alkyl-[N(CH₃)—C₁₋₆ alkyl]₁₋₈-N(CH₃)—*;
—C(O)—C₁₋₆ alkyl-[N(CH₃)C(O)—C₁₋₆ alkyl]₁₋₈-N(CH₃)—*;
A¹ is: 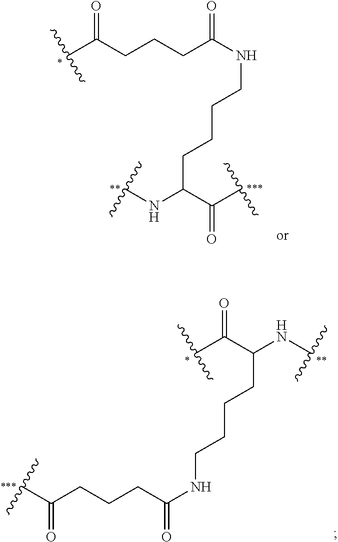 [SMILES: *NC(CCCCNC(=O)CCCC(*)=O)C(*)=O ; *NC(CCCCNC(=O)CCCC(*)=O)C(*)=O]
L² is C(O)—,
**C(O)—C₁₋₆ alkyl-NHC(O)—,
**C(O)—C₁₋₆ alkyl-[O—C₂₋₆ alkyl]₁₋₈-NHC(O)—,
**C(O)—C₁₋₆ alkyl-[N(CH₃)—C₁₋₆ alkyl]₁₋₈-NHC(O)—,
**C(O)—C₁₋₆ alkyl-[N(CH₃)C(O)—C₁₋₆ alkyl]₁₋₈-NHC(O)—,
**C(O)—C₁₋₆ alkyl-[N(CH₃)—C₁₋₆ alkyl]₁₋₈-N(CH₃)C(O)—;
**C(O)—C₁₋₆ alkyl-[N(CH₃)C(O)—C₁₋₆ alkyl]₁₋₈-N(CH₃)C(O)—;
L³ is —C₁₋₁₂ alkyl-,
***—C₁₋₁₂ alkyl-NH—,
***—NH—C₁₋₁₂ alkyl-,
***—NH—C₁₋₁₂ alkyl-NH—,
***—C(O)—C₁₋₁₂ alkyl-,
***—C(O)—C₁₋₁₂ alkyl-NH—;
***C(O)—C₁₋₆ alkyl-[O—C₂₋₆ alkyl]₁₋₈-NH—,
***C(O)—C₁₋₆ alkyl-[N(CH₃)—C₁₋₆ alkyl]₁₋₈-NH—,
***C(O)—C₁₋₆ alkyl-[N(CH₃)—C₁₋₆ alkyl]₁₋₈-N(CH₃)—;
***C(O)—C₁₋₆ alkyl-[N(CH₃)C(O)—C₁₋₆ alkyl]₁₋₈-N(CH₃)—;
wherein:
* denotes a bond from L¹ to A¹;
** denotes a bond from A¹ to L²,
*** denotes a bond from A¹ to L³.
R¹ is C₁₋₁₂ alkyl substituted with —NHL⁶-IN, —N(C₁₋₆alkyl)₂, or —N(C₁₋₆alkyl)₃⁺;
L⁶ is —C(O)—, —C₁₋₆ alkyl-, —C₁₋₆ alkyl-NH—, —NH—, or —NHC(O)—;
IN is an integrin binder; and
MOD is —COOH.

51. The compound of any one of claims 1, 6-13, 17-26, or 49-50, or a pharmaceutically acceptable salt thereof, or a stereoisomer or mixture of stereoisomers thereof, having the structure: 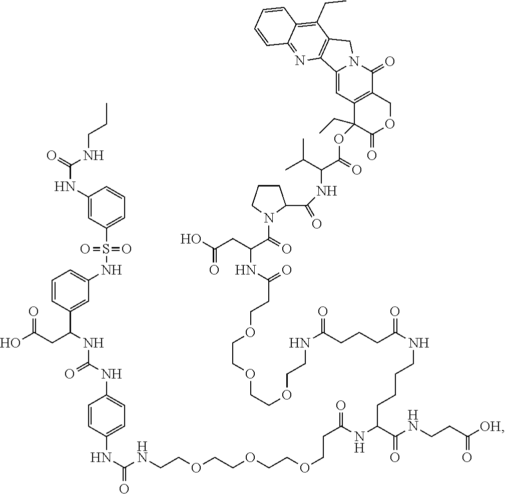 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CCCCNC(=O)CCCC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)C(=O)NCCC(=O)O)cc3)c2)c1] 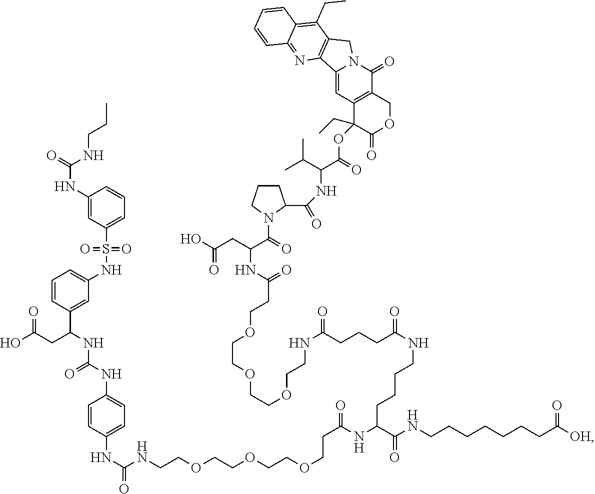 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CCCCNC(=O)CCCC(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)C(=O)NCCCCCCCC(=O)O)cc3)c2)c1] 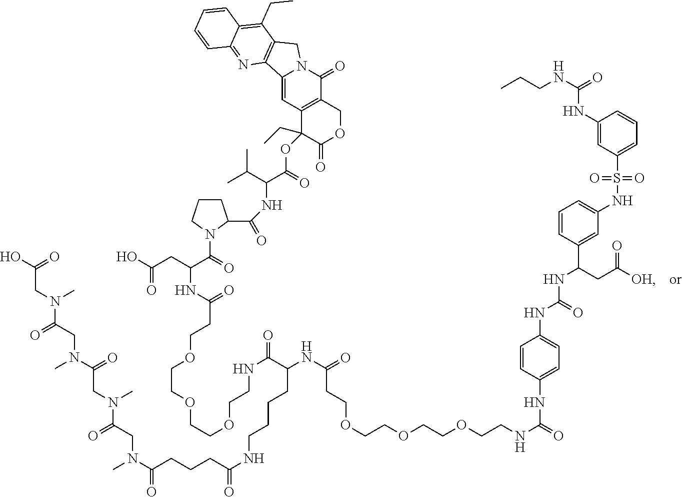 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CCCCNC(=O)CCCC(=O)N(C)CC(=O)N(C)CC(=O)N(C)CC(=O)N(C)CC(=O)O)C(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1] 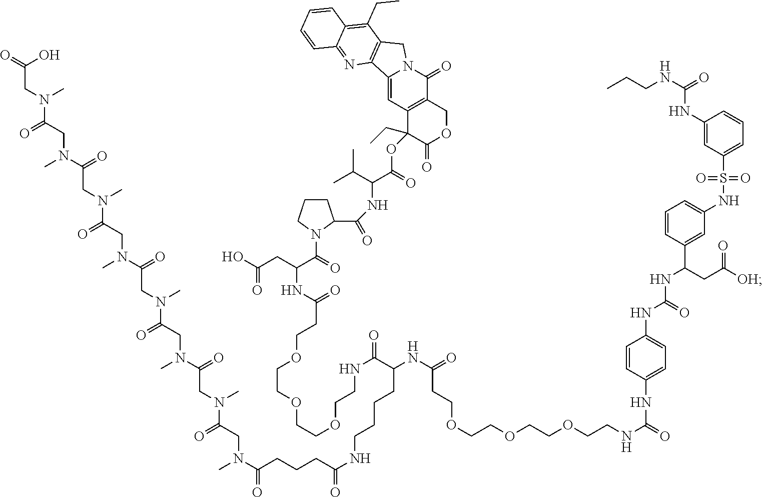 [SMILES: CCCNC(=O)Nc1cccc(S(=O)(=O)Nc2cccc(C(CC(=O)O)NC(=O)Nc3ccc(NC(=O)NCCOCCOCCOCCC(=O)NC(CCCCNC(=O)CCCC(=O)N(C)CC(=O)N(C)CC(=O)N(C)CC(=O)N(C)CC(=O)N(C)CC(=O)N(C)CC(=O)N(C)CC(=O)N(C)CC(=O)O)C(=O)NCCOCCOCCOCCC(=O)NC(CC(=O)O)C(=O)N4CCCC4C(=O)NC(C(=O)OC4(CC)C(=O)OCc5c4cc4n(c5=O)Cc5c-4nc4ccccc4c5CC)C(C)C)cc3)c2)c1]
or a pharmaceutically acceptable salt thereof, or a stereoisomer or mixture of stereoisomers thereof.

52. The compound of any one of claims 1-51, or a pharmaceutically acceptable salt thereof, or a stereoisomer or mixture of stereoisomers thereof, for the treatment of a disease or disorder.

53. The compound of claim 52, or a pharmaceutically acceptable salt thereof, or a stereoisomer or mixture of stereoisomers thereof, wherein the disease or disorder is a hyperproliferative disorder.

54. The compound of claim 52, or a pharmaceutically acceptable salt thereof, or a stereoisomer or mixture of stereoisomers thereof, wherein the disease or disorder is an autoimmune disorder.

55. The compound of claim 52, or a pharmaceutically acceptable salt thereof, or a stereoisomer or mixture of stereoisomers thereof, wherein the disease or disorder is an ophthalmological disease or disorder.

56. The compound of claim 55, or a pharmaceutically acceptable salt thereof, or a stereoisomer or mixture of stereoisomers thereof, wherein the ophthalmological disease or disorder is macular degeneration.

57. The compound of any one of claims 52-54, or a pharmaceutically acceptable salt thereof, or a stereoisomer or mixture of stereoisomers thereof, wherein the disease or disorder is a skin disorder.

58. The compound of claim 57, or a pharmaceutically acceptable salt thereof, or a stereoisomer or mixture of stereoisomers thereof, wherein the skin disorder is psoriasis.

59. The compound of any one of claim 53, or a pharmaceutically acceptable salt thereof, or a stereoisomer or mixture of stereoisomers thereof, wherein the hyperproliferative disorder is a cancer (e.g., an invasive and/or metastatic cancer).

60. The compound of claim 60 or a pharmaceutically acceptable salt thereof, or a stereoisomer or mixture of stereoisomers thereof, wherein the cancer is of the breast, respiratory tract, brain, reproductive organs, digestive tract, urinary tract, eye, liver, skin, head and neck, thyroid, parathyroid or a metastasis thereof.

61. The compound of claim 60, or a pharmaceutically acceptable salt thereof, or a stereoisomer or mixture of stereoisomers thereof, wherein the cancer of the breast is invasive ductal carcinoma, invasive lobular carcinoma, ductal carcinoma in situ, lobular carcinoma in situ, inflammatory breast cancer, basal breast cancer, or triple negative breast cancer.

62. The compound of claim 60, or a pharmaceutically acceptable salt thereof, or a stereoisomer or mixture of stereoisomers thereof, wherein the cancer of the respiratory tract is small-cell lung carcinoma, non-small-cell lung carcinoma, bronchial adenoma, or pleuropulmonary blastoma.

63. The compound of claim 60, or a pharmaceutically acceptable salt thereof, or a stereoisomer or mixture of stereoisomers thereof, wherein the cancer of the brain is a brain stem glioma, hypothalmic glioma, glioblastoma, cerebellar astrocytoma, cerebral astrocytoma, medulloblastoma, ependymoma, neuroectodermal tumor, or pineal tumor.

64. The compound of claim 60, or a pharmaceutically acceptable salt thereof, or a stereoisomer or mixture of stereoisomers thereof, wherein the cancer of the reproductive organs is a prostate cancer, testicular cancer, endometrial cancer, cervical cancer, ovarian cancer, vaginal cancer, vulvar cancer, or a sarcoma of the uterus.

65. The compound of claim 60, or a pharmaceutically acceptable salt thereof, or a stereoisomer or mixture of stereoisomers thereof, wherein the cancer of the digestive tract is anal cancer, colon cancer, colorectal cancer, esophageal cancer, gallbladder cancer, gastric cancer, pancreatic cancer, rectal cancer, small intestinal cancer, or salivary gland cancer.

66. The compound of claim 60, or a pharmaceutically acceptable salt thereof, or a stereoisomer or mixture of stereoisomers thereof, wherein the cancer of the urinary tract is bladder cancer, penile cancer, kidney cancer, renal pelvis cancer, ureter cancer, or urethral cancer.

67. The compound of claim 60, or a pharmaceutically acceptable salt thereof, or a stereoisomer or mixture of stereoisomers thereof, wherein the cancer of the eye is intraocular melanoma or retinoblastoma.

68. The compound of claim 60, or a pharmaceutically acceptable salt thereof, or a stereoisomer or mixture of stereoisomers thereof, wherein the cancer of the liver is hepatocellular carcinoma, liver cell carcinoma with or without fibrolamellar variant, cholangiocarcinoma, intrahepatic bile duct carcinoma, or mixed hepatocellular cholangiocarcinoma.

69. The compound of claim 60, or a pharmaceutically acceptable salt thereof, or a stereoisomer or mixture of stereoisomers thereof, wherein the cancer of the skin is squamous cell carcinoma, Kaposi's sarcoma, malignant melanoma, Merkel cell skin cancer, or non-melanoma skin cancer.

70. The compound of claim 60, or a pharmaceutically acceptable salt thereof, or a stereoisomer or mixture of stereoisomers thereof, wherein the cancer of the head-and-neck is laryngeal cancer, hypopharyngeal cancer, nasopharyngeal cancer, oropharyngeal cancer, or a lip and oral cavity cancer.

71. The compound of claim 59, or a pharmaceutically acceptable salt thereof, or a stereoisomer or mixture of stereoisomers thereof, wherein the cancer is a sarcoma.

72. The compound of claim 71, or a pharmaceutically acceptable salt thereof, or a stereoisomer or mixture of stereoisomers thereof, wherein the sarcoma is Ewing sarcoma, osteosarcoma, or fibrosarcoma.

73. A pharmaceutical composition comprising a compound of any one of 5 claims 1-51, or a pharmaceutically acceptable salt thereof, or a stereoisomer or mixture of stereoisomers thereof, and a pharmaceutically acceptable excipient.

74. A method of treating a disease or disorder in a subject, comprising administering to the subject a therapeutically effective amount of a compound of any one of claims 1-51, or a pharmaceutically acceptable salt thereof, or a stereoisomer or mixture of stereoisomers thereof, or the pharmaceutical composition of claim 73, to an individual in need thereof.

75. The method of claim 74, wherein the disease or disorder is a hyperproliferative disorder.

76. The method of claim 74, wherein the disease or disorder is a cancer.

77. The method of claim 76, wherein the cancer is breast cancer, colon cancer, renal cancer, or lung cancer.
